(19) United States
(12) Patent Application Publication
(10) Pub. No.: US 2024/0279362 A1
(43) Pub. Date: Aug. 22, 2024

(54) TRISPECIFIC AND/OR TRIVALENT BINDING PROTEINS

(71) Applicant: Sanofi, Paris (FR)

(72) Inventors: Zhi-Yong YANG, Cambridge, MA (US); Gary J. NABEL, Cambridge, MA (US); Lan WU, Cambridge, MA (US); Edward SEUNG, Cambridge, MA (US); Ronnie WEI, Needham, MA (US); Jochen BENINGA, Frankfurt Am Main (DE); Ercole RAO, Morfelden-Walldorf (DE); Wulf Dirk LEUSCHNER, Frankfurt Am Main (DE); Christian BEIL, Frankfurt Am Main (DE); Christian LANGE, Frankfurt Am Main (DE); Carsten CORVEY, Frankfurt Am Main (DE)

(21) Appl. No.: 18/433,295

(22) Filed: Feb. 5, 2024

Related U.S. Application Data
(63) Continuation of application No. 17/515,227, filed on Oct. 29, 2021, now Pat. No. 11,932,704.
Application No. 17/515,227 is a continuation of application No. 17/099,439, filed on Nov. 16, 2020, now Pat. No. 11,192,960.
Application No. 17/099,439 is a continuation of application No. 15/487,243, filed on Apr. 13, 2017, now Pat. No. 10,882,922.
(60) Provisional application No. 62/412,187, filed on Oct. 24, 2016.
(60) Provisional application No. 62/331,191, filed on May 3, 2016.
(60) Provisional application No. 62/322,036, filed on Apr. 13, 2016.

(30) Foreign Application Priority Data
Mar. 17, 2017 (EP) 17305298.6

(51) Int. Cl. C07K 16/46; A61K 39/00; A61K 39/395; A61K 45/06; C07K 1/22; C07K 16/24; C07K 16/28; C07K 16/32

(52) U.S. Cl. CPC C07K 16/468; A61K 39/3955; A61K 39/39558; A61K 45/06; C07K 1/22; C07K 16/241; C07K 16/244; C07K 16/247; C07K 16/2803; C07K 16/2809; C07K 16/2818; C07K 16/2887; C07K 16/2896; C07K 16/32; A61K 2039/505; C07K 2317/31; C07K 2317/35; C07K 2317/52; C07K 2317/526; C07K 2317/56; C07K 2317/565; C07K 2317/64; C07K 2317/73; C07K 2317/75; C07K 2317/76; C07K 2317/92; C07K 2317/94; C07K 2319/00

(57) ABSTRACT

The disclosure provides trispecific and/or trivalent binding proteins comprising four polypeptide chains that form three antigen binding sites that specifically bind one or more target proteins, wherein a first pair of polypeptides forming the binding protein possess dual variable domains having a cross-over orientation and wherein a second pair of polypeptides forming the binding protein possess a single variable domain. The disclosure also provides methods for making trispecific and/or trivalent binding proteins and uses of such binding proteins.

### CROSS REFERENCES TO RELATED APPLICATIONS

This application claims the priority benefit of U.S. Provisional Application Ser. No. 62/322,036, filed Apr. 13, 2016; U.S. Provisional Application Ser. No. 62/331,191, filed May 3, 2016; U.S. Provisional Application Ser. No. 62/412,187, filed Oct. 24, 2016; and EP Application No. EP17305298.6, filed Mar. 17, 2017; which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 183952027100SEQLISTING.txt, date recorded: Apr. 12, 2017, size: 200 KB).

### FIELD OF THE INVENTION

The disclosure relates to trispecific and/or trivalent binding proteins comprising four polypeptide chains that form three antigen binding sites that specifically bind one or more target proteins, wherein a first pair of polypeptides forming the binding protein possess dual variable domains having a cross-over orientation and wherein a second pair of polypeptides forming the binding protein possess a single variable domain. The disclosure also relates to methods for making trispecific and/or trivalent binding proteins and uses of such binding proteins.

### BACKGROUND

Monoclonal antibody based biotherapeutics have become an important avenue for new drug development. Monoclonal antibody technology offers specific targeting, precise signaling delivery and/or payload to specific cell population, and provides long lasting biological effect through its Fc functions. Efforts in antibody engineering have allowed developing bispecific antibodies combining the specificities of two monoclonal antibodies for various biological applications, expanding the scope of antibody drug development. Newly discovered neutralizing antibodies with improved breadth and potency may provide more options for developing biotherapeutics to treat complexed diseases such as cancer, arthritis, and/or inflammatory disorders.

### BRIEF SUMMARY

Provided herein are multispecific binding proteins (e.g., antibodies) that form three antigen binding sites. These binding proteins can specifically bind one, two, or three antigen targets or target proteins.

In one embodiment, the disclosure provides a binding protein comprising four polypeptide chains that form three antigen binding sites that specifically bind one or more antigen targets or target proteins, wherein a first polypeptide chain has a structure represented by the formula:

V_{L2}-L₁-V_{L1}-L₂-C_{L} [I]

and a second polypeptide chain has a structure represented by the formula:

V_{H1}-L₃-V_{H2}-L₄-C_{H1} [II]

and a third polypeptide chain has a structure represented by the formula:

V_{H3}-C_{H1} [III]

and a fourth polypeptide chain has a structure represented by the formula:

V_{L3}-C_{L} [IV]

wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain; and
L₁, L₂, L₃ and L₄ are amino acid linkers;
   and wherein the polypeptide of formula I and the polypeptide of formula II form a cross-over light chain-heavy chain pair.

In some embodiments, the second and/or third polypeptide chain further comprises an Fc region linked to C_{H1}, the Fc region comprising an immunoglobulin hinge region and C_{H2} and C_{H3} immunoglobulin heavy chain constant domains.

In another embodiment, the disclosure provides a binding protein comprising four polypeptide chains that form three antigen binding sites that specifically bind one or more target proteins, wherein a first polypeptide chain comprises a structure represented by the formula:

V_{L2}-L₁-V_{L1}-L₂-C_{L} [I]

and a second polypeptide chain comprises a structure represented by the formula:

V_{H1}-L₃-V_{H2}-L₄-C_{H1}-hinge-C_{H2}-C_{H3} [II]

and a third polypeptide chain comprises a structure represented by the formula:

V_{H3}-C_{H1}-hinge-C_{H2}-C_{H3} [III]

and a fourth polypeptide chain comprises a structure represented by the formula:

V_{L3}-C_{L} [IV]

wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain;
C_{H2} is an immunoglobulin C_{H2} heavy chain constant domain;
C_{H3} is an immunoglobulin C_{H3} heavy chain constant domain;
hinge is an immunoglobulin hinge region connecting the C_{H1} and C_{H2} domains; and
L₁, L₂, L₃ and L₄ are amino acid linkers;
and wherein the polypeptide of formula I and the polypeptide of formula II form a cross-over light chain-heavy chain pair.

In some embodiments, the binding protein is trispecific and capable of specifically binding three different antigen targets. In some embodiments, the binding protein is trivalent but bispecific and capable of specifically binding three antigen targets, two of them being identical. In some embodiments, the binding protein of the present disclosure is trivalent but monospecific and capable of specifically binding three antigen targets, all of them being identical. In some embodiments, the binding protein is capable of inhibiting the function of one or more target proteins. In some embodiments, the binding protein is trispecific and capable of specifically binding three different antigen targets.

In some embodiments, a binding protein of the present disclosure comprises one, two, or three antigen binding sites that specifically bind a target protein selected from A2AR, APRIL, ATPDase, BAFF, BAFFR, BCMA, BlyS, BTK, BTLA, B7DC, B7H1, B7H4 (also known as VTCN1), B7H5, B7H6, B7H7, B7RP1, B7-4, C3, C5, CCL2 (also known as MCP-1), CCL3 (also known as MIP-1a), CCL4 (also known as MIP-1b), CCL5 (also known as RANTES), CCL7 (also known as MCP-3), CCL8 (also known as mcp-2), CCL11 (also known as eotaxin), CCL15 (also known as MIP-1d), CCL17 (also known as TARC), CCL19 (also known as MIP-3b), CCL20 (also known as MIP-3a), CCL21 (also known as MIP-2), CCL24 (also known as MPIF-2/eotaxin-2), CCL25 (also known as TECK), CCL26 (also known as eotaxin-3), CCR3, CCR4, CD3, CD19, CD20, CD23 (also known as FCER2, a receptor for IgE), CD24, CD27, CD28, CD38, CD39, CD40, CD70, CD80 (also known as B7-1), CD86 (also known as B7-2), CD122, CD137 (also known as 41BB), CD137L, CD152 (also known as CTLA4), CD154 (also known as CD40L), CD160, CD272, CD273 (also known as PDL2), CD274 (also known as PDL1), CD275 (also known as B7H2), CD276 (also known as B7H3), CD278 (also known as ICOS), CD279 (also known as PD-1), CDH1 (also known as E-cadherin), chitinase, CLEC9, CLEC91, CRTH2, CSF-1 (also known as M-CSF), CSF-2 (also known as GM-CSF), CSF-3 (also known as GCSF), CX3CL1 (also known as SCYD1), CXCL12 (also known as SDF1), CXCL13, CXCR3, DNGR-1, ectonucleoside triphosphate diphosphohydrolase 1, EGFR, ENTPD1, FCER1A, FCER1, FLAP, FOLH1, Gi24, GITR, GITRL, GM-CSF, Her2, HHLA2, HMGB1, HVEM, ICOSLG, IDO, IFNα, IgE, IGF1R, IL2Rbeta, IL1, IL1A, IL1B, IL1F10, IL2, IL4, IL4Ra, IL5, IL5R, IL6, IL7, IL7Ra, IL8, IL9, IL9R, IL10, rhIL10, IL12, IL13, IL13Ra1, IL13Ra2, IL15, IL17, IL17Rb (also known as a receptor for IL25), IL18, IL22, IL23, IL25, IL27, IL33, IL35, ITGB4 (also known as b4 integrin), ITK, KIR, LAG3, LAMP1, leptin, LPFS2, MHC class II, NCR3LG1, NKG2D, NTPDase-1, OX40, OX40L, PD-1H, platelet receptor, PROM1, S152, SISP1, SLC, SPG64, ST2 (also known as a receptor for IL33), STEAP2, Syk kinase, TACI, TDO, T14, TIGIT, TIM3, TLR, TLR2, TLR4, TLR5, TLR9, TMEF1, TNFa, TNFRSF7, Tp55, TREM1, TSLP (also known as a co-receptor for IL7Ra), TSLPR, TWEAK, VEGF, VISTA, Vstm3, WUCAM, and XCR1 (also known as GPR5/CCXCR1). In some embodiments, one or more of the above antigen targets are human antigen targets. In some embodiments, the binding protein of the present disclosure is trispecific and capable of specifically binding three different antigen targets selected from the above list. In some embodiments, the binding protein of the present disclosure is trivalent but bispecific and capable of specifically binding three antigen targets selected from the above list, two of them being identical. In some embodiments, the binding protein of the present disclosure is trivalent but monospecific and capable of specifically binding three antigen targets selected from the above list, all of them being identical. In some embodiments, the binding protein specifically binds three target proteins that correspond to two target proteins on T cells and to one tumor target protein. In some embodiments, one of said target proteins on T cells is CD3. In some embodiments, one of said target proteins on T cells is CD28. In some embodiments, said tumor target protein is CD38. In some embodiments, the binding protein specifically binds three target proteins that correspond to two target proteins on T cells and to one target protein selected from the group consisting of A2AR, APRIL, ATPDase, BAFF, BAFFR, BCMA, BlyS, BTK, BTLA, B7DC, B7H1, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL24, CCL25, CCL26, CCR3, CCR4, CD3, CD19, CD20, CD23, CD24, CD27, CD28, CD38, CD39, CD40, CD70, CD80, CD86, CD122, CD137, CD137L, CD152, CD154, CD160, CD272, CD273, CD274, CD275, CD276, CD278, CD279, CDH1, chitinase, CLEC9, CLEC91, CRTH2, CSF-1, CSF-2, CSF-3, CX3CL1, CXCL12, CXCL13, CXCR3, DNGR-1, ectonucleoside triphosphate diphosphohydrolase 1, EGFR, ENTPD1, FCER1A, FCER1, FLAP, FOLH1, Gi24, GITR, GITRL, GM-CSF, Her2, HHLA2, HMGB1, HVEM, ICOSLG, IDO, IFNα, IgE, IGF1R, IL2Rbeta, IL1, IL1A, IL1B, IL1F10, IL2, IL4, IL4Ra, IL5, IL5R, IL6, IL7, IL7Ra, IL8, IL9, IL9R, IL10, rhIL10, IL12, IL13, IL13Ra1, IL13Ra2, IL15, IL17, IL17Rb, IL18, IL22, IL23, IL25, IL27, IL33, IL35, ITGB4, ITK, KIR, LAG3, LAMP1, leptin, LPFS2, MHC class II, NCR3LG1, NKG2D, NTPDase-1, OX40, OX40L, PD-1H, platelet receptor, PROM1, S152, SISP1, SLC, SPG64, ST2, STEAP2, Syk kinase, TACI, TDO, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, WUCAM, and XCR1.

In another embodiment, the disclosure provides a binding protein comprising four polypeptide chains that form three antigen binding sites, wherein a first polypeptide chain has a structure represented by the formula:

V_{L2}-L₁-V_{L1}-L₂-C_{L} [I]

and a second polypeptide chain has a structure represented by the formula:

V_{H1}-L₃-V_{H2}-L₄-C_{H1} [II]

and a third polypeptide chain has a structure represented by the formula:

V_{H3}-C_{H1}-hinge-C_{H2}-C_{H3} [III]

and a fourth polypeptide chain has a structure represented by the formula:

V_{L3}-C_{L} [IV]

wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain; and
L₁, L₂, L₃ and L₄ are amino acid linkers;
wherein the polypeptide of formula I and the polypeptide of formula II form a cross-over light chain-heavy chain pair;
wherein:
(a) V_{L1}, V_{L2} and V_{L3} are each independently a variable domain derived from an amino acid sequence as set forth in any one of SEQ ID NOs: 2, 4, 10, 14, 18, 22, 115;
(b) V_{L1}, V_{L2} and V_{L3} each independently comprise light chain complementarity determining regions of a variable domain comprising an amino acid sequence as set forth in any one of SEQ ID NOs: 43-59, 123-125;
(c) V_{L1}, V_{L2} and V_{L3} each independently comprise a variable domain sequence as set forth in any one of SEQ ID NOs:151, 153, 155, 157, 159, 161, 163, 165, and 167;
(d) V_{L1}, V_{L2} and V_{L3} each independently comprise light chain complementarity determining regions comprising an amino acid sequence as set forth in any one of SEQ ID NOs: 43-59, 123-125, 138-140, and 149; or
(e) V_{L1}, V_{L2} and V_{L3} each independently comprise light chain complementarity determining regions and/or a variable domain sequence shown in Tables 2-5; and
wherein:
(a) V_{H1}, V_{H2}, and V_{H3} are each independently a variable domain derived from an amino acid sequence as set forth in any one of SEQ ID NOs: 1, 3, 9, 13, 17, 21, 114;
(b) V_{H1}, V_{H2} and V_{H3} each independently comprise heavy chain complementarity determining regions of a variable domain comprising an amino acid sequence as set forth in any one of SEQ ID NOs: 25-42, 120-122;
(c) V_{H1}, V_{H2}, and V_{H3} each independently comprise a variable domain sequence as set forth in any one of SEQ ID NOs:150, 152, 154, 156, 158, 160, 162, 164, and 166;
(d) V_{H1}, V_{H2} and V_{H3} each independently comprise heavy chain complementarity determining regions comprising an amino acid sequence as set forth in any one of SEQ ID NOs: 25-42, 120-122, and 126-128; or
(e) V_{H1}, V_{H2} and V_{H3} each independently comprise heavy chain complementarity determining regions and/or a variable domain sequence shown in Tables 2-5.

In some embodiments, the second and/or third polypeptide chain further comprises an Fc region linked to C_{H1}, the Fc region comprising an immunoglobulin hinge region and C_{H2} and C_{H3} immunoglobulin heavy chain constant domains.

In another embodiment, the disclosure provides a binding protein comprising four polypeptide chains that form three antigen binding sites, wherein a first polypeptide chain comprises a structure represented by the formula:

V_{L2}-L₁-V_{L1}-L₂-C_{L} [I]

and a second polypeptide chain comprises a structure represented by the formula:

V_{H1}-L₃-V_{H2}-L₄-C_{H1}-hinge-C_{H2}-C_{H3} [II]

and a third polypeptide chain comprises a structure represented by the formula:

V_{H3}-C_{H1}-hinge-C_{H2}-C_{H3} [III]

and a fourth polypeptide chain comprises a structure represented by the formula:

V_{L3}-C_{L} [IV]

wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain;
C_{H2} is an immunoglobulin C_{H2} heavy chain constant domain;
C_{H3} is an immunoglobulin C_{H3} heavy chain constant domain;
hinge is an immunoglobulin hinge region connecting the C_{H1} and C_{H2} domains; and
L₁, L₂, L₃ and L₄ are amino acid linkers;
wherein the polypeptide of formula I and the polypeptide of formula II form a cross-over light chain-heavy chain pair;
wherein:
(a) V_{L1}, V_{L2} and V_{L3} are each independently a variable domain derived from an amino acid sequence as set forth in any one of SEQ ID NOs: 2, 4, 10, 14, 18, 22, 115;
(b) V_{L1}, V_{L2} and V_{L3} each independently comprise light chain complementarity determining regions of a variable domain comprising an amino acid sequence as set forth in any one of SEQ ID NOs: 43-59, 123-125;
(c) V_{L1}, V_{L2} and V_{L3} each independently comprise a variable domain sequence as set forth in any one of SEQ ID NOs:151, 153, 155, 157, 159, 161, 163, 165, and 167;
(d) V_{L1}, V_{L2} and V_{L3} each independently comprise light chain complementarity determining regions comprising an amino acid sequence as set forth in any one of SEQ ID NOs: 43-59, 123-125, 138-140, and 149; or
(e) V_{L1}, V_{L2} and V_{L3} each independently comprise light chain complementarity determining regions and/or a variable domain sequence shown in Tables 2-5;
wherein:
(a) V_{H1}, V_{H2}, and V_{H3} are each independently a variable domain derived from an amino acid sequence as set forth in any one of SEQ ID NOs: 1, 3, 9, 13, 17, 21, 114;
(b) V_{H1}, V_{H2} and V_{H3} each independently comprise heavy chain complementarity determining regions of a variable domain comprising an amino acid sequence as set forth in any one of SEQ ID NOs: 25-42, 120-122;
(c) V_{H1}, V_{H2}, and V_{H3} each independently comprise a variable domain sequence as set forth in any one of SEQ ID NOs:150, 152, 154, 156, 158, 160, 162, 164, and 166;
(d) V_{H1}, V_{H2} and V_{H3} each independently comprise heavy chain complementarity determining regions comprising an amino acid sequence as set forth in any one of SEQ ID NOs: 25-42, 120-122, and 126-128; or
(e) V_{H1}, V_{H2} and V_{H3} each independently comprise heavy chain complementarity determining regions and/or a variable domain sequence shown in Tables 2-5.

In some embodiments, V_{L1}, V_{L2} and V_{L3} each independently comprise a variable domain sequence as set forth in any one of SEQ ID NOs:151, 153, 155, 157, 159, 161, 163, 165, and 167; and V_{H1}, V_{H2}, and V_{H3} each independently comprise a variable domain sequence as set forth in any one of SEQ ID NOs:150, 152, 154, 156, 158, 160, 162, 164, and 166. In some embodiments, V_{L1}, V_{L2} and V_{L3} each independently comprise light chain complementarity determining regions comprising an amino acid sequence as set forth in any one of SEQ ID NOs: 43-59, 123-125, 138-140, and 149; and (d) V_{H1}, V_{H2} and V_{H3} each independently comprise heavy chain complementarity determining regions comprising an amino acid sequence as set forth in any one of SEQ ID NOs: 25-42, 120-122, and 126-128.

In some embodiments of any of the binding proteins described herein, (a) V_{H1} comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:30; V_{L1} comprises a CDR-L1 comprising the amino acid sequence of SEQ ID NO:46, a CDR-L2 comprising the amino acid sequence of SEQ ID NO: 47, and a CDR-L3 comprising the amino acid sequence of SEQ ID NO:48; V_{H2} Comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} comprises a CDR-H1 comprising the amino acid sequence of SEQ ID NO:25, a CDR-H2 comprising the amino acid sequence of SEQ ID NO:26, and a CDR-H3 comprising the amino acid sequence of SEQ ID NO:27; and V_{L3} comprises a CDR-L1 comprising the amino acid sequence of SEQ ID NO:43, a CDR-L2 comprising the amino acid sequence of SEQ ID NO:44, and a CDR-L3 comprising the amino acid sequence of SEQ ID NO:45; (b) V_{H1} comprises a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33; V_{L1} comprises a CDR-L1 comprising the amino acid sequence of SEQ ID NO:49, a CDR-L2 comprising the amino acid sequence of SEQ ID NO:50, and a CDR-L3 comprising the amino acid sequence of SEQ ID NO:51; V_{H2} comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} Comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} comprises a CDR-H1 comprising the amino acid sequence of SEQ ID NO:25, a CDR-H2 comprising the amino acid sequence of SEQ ID NO:26, and a CDR-H3 comprising the amino acid sequence of SEQ ID NO:27; and V_{L3} comprises a CDR-L1 comprising the amino acid sequence of SEQ ID NO:43, a CDR-L2 comprising the amino acid sequence of SEQ ID NO:44, and a CDR-L3 comprising the amino acid sequence of SEQ ID NO:45; (c) V_{H1} comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:30; V_{L1} comprises a CDR-L1 comprising the amino acid sequence of SEQ ID NO:46, a CDR-L2 comprising the amino acid sequence of SEQ ID NO: 47, and a CDR-L3 comprising the amino acid sequence of SEQ ID NO:48; V_{H2} Comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} comprises a CDR-H1 comprising the amino acid sequence of SEQ ID NO:37, a CDR-H2 comprising the amino acid sequence of SEQ ID NO:38, and a CDR-H3 comprising the amino acid sequence of SEQ ID NO:39; and V_{L3} comprises a CDR-L1 comprising the amino acid sequence of SEQ ID NO:55, a CDR-L2 comprising the amino acid sequence of SEQ ID NO:56, and a CDR-L3 comprising the amino acid sequence of SEQ ID NO:57; (d) V_{H1} comprises a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33; V_{L1} comprises a CDR-L1 comprising the amino acid sequence of SEQ ID NO:49, a CDR-L2 comprising the amino acid sequence of SEQ ID NO:50, and a CDR-L3 comprising the amino acid sequence of SEQ ID NO:51; V_{H2} comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} Comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} comprises a CDR-H1 comprising the amino acid sequence of SEQ ID NO:37, a CDR-H2 comprising the amino acid sequence of SEQ ID NO:38, and a CDR-H3 comprising the amino acid sequence of SEQ ID NO:39; and V_{L3} comprises a CDR-L1 comprising the amino acid sequence of SEQ ID NO:55, a CDR-L2 comprising the amino acid sequence of SEQ ID NO:56, and a CDR-L3 comprising the amino acid sequence of SEQ ID NO:57; (e) V_{H1} comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:30; V_{L1} comprises a CDR-L1 comprising the amino acid sequence of SEQ ID NO:46, a CDR-L2 comprising the amino acid sequence of SEQ ID NO: 47, and a CDR-L3 comprising the amino acid sequence of SEQ ID NO:48; V_{H2} Comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} comprises a CDR-H1 comprising the amino acid sequence of SEQ ID NO:40, a CDR-H2 comprising the amino acid sequence of SEQ ID NO:41, and a CDR-H3 comprising the amino acid sequence of SEQ ID NO:42; and V_{L3} comprises a CDR-L1 comprising the amino acid sequence of SEQ ID NO:58, a CDR-L2 comprising the amino acid sequence of SEQ ID NO:44, and a CDR-L3 comprising the amino acid sequence of SEQ ID NO:59; (f) V_{H1} comprises a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33; V_{L1} comprises a CDR-L1 comprising the amino acid sequence of SEQ ID NO:49, a CDR-L2 comprising the amino acid sequence of SEQ ID NO:50, and a CDR-L3 comprising the amino acid sequence of SEQ ID NO:51; V_{H2} comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} Comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} comprises a CDR-H1 comprising the amino acid sequence of SEQ ID NO:40, a CDR-H2 comprising the amino acid sequence of SEQ ID NO:41, and a CDR-H3 comprising the amino acid sequence of SEQ ID NO:42; and V_{L3} comprises a CDR-L1 comprising the amino acid sequence of SEQ ID NO:58, a CDR-L2 comprising the amino acid sequence of SEQ ID NO:44, and a CDR-L3 comprising the amino acid sequence of SEQ ID NO:59; (g) V_{H1} comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:30; V_{L1} comprises a CDR-L1 comprising the amino acid sequence of SEQ ID NO:46, a CDR-L2 comprising the amino acid sequence of SEQ ID NO: 47, and a CDR-L3 comprising the amino acid sequence of SEQ ID NO:48; V_{H2} Comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} comprises a CDR-H1 comprising the amino acid sequence of SEQ ID NO:126, a CDR-H2 comprising the amino acid sequence of SEQ ID NO:127, and a CDR-H3 comprising the amino acid sequence of SEQ ID NO:128; and V_{L3} comprises a CDR-L1 comprising the amino acid sequence of SEQ ID NO:138, a CDR-L2 comprising the amino acid sequence of SEQ ID NO:139, and a CDR-L3 comprising the amino acid sequence of SEQ ID NO:140; (h) V_{H1} comprises a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33; V_{L1} comprises a CDR-L1 comprising the amino acid sequence of SEQ ID NO:49, a CDR-L2 comprising the amino acid sequence of SEQ ID NO:50, and a CDR-L3 comprising the amino acid sequence of SEQ ID NO:51; V_{H2} comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} comprises a CDR-H1 comprising the amino acid sequence of SEQ ID NO:126, a CDR-H2 comprising the amino acid sequence of SEQ ID NO:127, and a CDR-H3 comprising the amino acid sequence of SEQ ID NO:128; and V_{L3} Comprises a CDR-L1 comprising the amino acid sequence of SEQ ID NO:138, a CDR-L2 comprising the amino acid sequence of SEQ ID NO:139, and a CDR-L3 comprising the amino acid sequence of SEQ ID NO:140; (i) V_{H1} comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:30; V_{L1} comprises a CDR-L1 comprising the amino acid sequence of SEQ ID NO:46, a CDR-L2 comprising the amino acid sequence of SEQ ID NO: 47, and a CDR-L3 comprising the amino acid sequence of SEQ ID NO:48; V_{H2} comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} comprises a CDR-H1 comprising the amino acid sequence of SEQ ID NO:120, a CDR-H2 comprising the amino acid sequence of SEQ ID NO:121, and a CDR-H3 comprising the amino acid sequence of SEQ ID NO:122; and V_{L3} comprises a CDR-L1 comprising the amino acid sequence of SEQ ID NO:123, a CDR-L2 comprising the amino acid sequence of SEQ ID NO:124, and a CDR-L3 comprising the amino acid sequence of SEQ ID NO:125; or (j) V_{H1} comprises a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33; V_{L1} comprises a CDR-L1 comprising the amino acid sequence of SEQ ID NO:49, a CDR-L2 comprising the amino acid sequence of SEQ ID NO:50, and a CDR-L3 comprising the amino acid sequence of SEQ ID NO:51; V_{H2} comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} comprises a CDR-H1 comprising the amino acid sequence of SEQ ID NO:120, a CDR-H2 comprising the amino acid sequence of SEQ ID NO:121, and a CDR-H3 comprising the amino acid sequence of SEQ ID NO:122; and V_{L3} Comprises a CDR-L1 comprising the amino acid sequence of SEQ ID NO:123, a CDR-L2 comprising the amino acid sequence of SEQ ID NO:124, and a CDR-L3 comprising the amino acid sequence of SEQ ID NO:125. In some embodiments, (a) V_{H1} comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L1} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H2} comprises a CDR-H1 comprising the amino acid sequence of SEQ ID NO:25, a CDR-H2 comprising the amino acid sequence of SEQ ID NO:26, and a CDR-H3 comprising the amino acid sequence of SEQ ID NO:27; V_{L2} Comprises a CDR-L1 comprising the amino acid sequence of SEQ ID NO:43, a CDR-L2 comprising the amino acid sequence of SEQ ID NO:44, and a CDR-L3 comprising the amino acid sequence of SEQ ID NO:45; V_{H3} comprises a CDR-H1 comprising the amino acid sequence of SEQ ID NO:25, a CDR-H2 comprising the amino acid sequence of SEQ ID NO:26, and a CDR-H3 comprising the amino acid sequence of SEQ ID NO:27; and V_{L3} comprises a CDR-L1 comprising the amino acid sequence of SEQ ID NO:43, a CDR-L2 comprising the amino acid sequence of SEQ ID NO:44, and a CDR-L3 comprising the amino acid sequence of SEQ ID NO:45.

In some embodiments, the binding protein comprises one antigen binding site that specifically binds a T-cell surface protein and another antigen binding site that specifically binds an antigen target, e.g., a tumor target protein. In some embodiments, the binding protein comprises an antigen binding site that specifically binds CD3, an antigen binding site that specifically binds CD28, and an antigen binding site that specifically binds a tumor target protein selected from the group consisting of CD19, CD20, CD38, Her2, and LAMP1. In some embodiments, V_{H1} and V_{L1} form a first antigen binding site that specifically binds human CD3, V_{H2} and V_{L2} form a second antigen binding site that specifically binds human CD28, and V_{H3} and V_{L3} form a third antigen binding site that specifically binds a human tumor target protein. In some embodiments, V_{H1} and V_{L1} form a first antigen binding site that specifically binds human CD28, V_{H2} and V_{L2} form a second antigen binding site that specifically binds human CD3, and V_{H3} and V_{L3} form a third binding site that specifically binds a human tumor target protein. In some embodiments, the antigen binding site specifically binds a human tumor target protein selected from the group consisting of CD19, CD20, CD38, Her2, and LAMP1. In some embodiments, the antigen binding site that specifically binds CD3 comprises: (a) a heavy chain variable domain comprising the amino acid sequence of SEQ ID NO: 152 and a light chain variable domain comprising the amino acid sequence of SEQ ID NO: 153; or (b) a heavy chain variable domain comprising the amino acid sequence of SEQ ID NO: 154 and a light chain variable domain comprising the amino acid sequence of SEQ ID NO: 155. In some embodiments, the antigen binding site that specifically binds CD3 comprises six CDRs, or a heavy chain and a light chain variable domain, shown in Tables 2-5. In some embodiments, the antigen binding site that specifically binds CD28 comprises: (a) a heavy chain variable domain comprising the amino acid sequence of SEQ ID NO: 160 and a light chain variable domain comprising the amino acid sequence of SEQ ID NO: 161; or (b) a heavy chain variable domain comprising the amino acid sequence of SEQ ID NO: 162 and a light chain variable domain comprising the amino acid sequence of SEQ ID NO: 163. In some embodiments, the antigen binding site that specifically binds CD28 comprises six CDRs, or a heavy chain and a light chain variable domain, shown in Tables 2-5. In some embodiments, the antigen binding site that specifically binds a tumor target protein comprises: (a) a heavy chain variable domain comprising the amino acid sequence of SEQ ID NO: 156 and a light chain variable domain comprising the amino acid sequence of SEQ ID NO: 157; (b) a heavy chain variable domain comprising the amino acid sequence of SEQ ID NO: 158 and a light chain variable domain comprising the amino acid sequence of SEQ ID NO: 159; (c) a heavy chain variable domain comprising the amino acid sequence of SEQ ID NO: 164 and a light chain variable domain comprising the amino acid sequence of SEQ ID NO: 165; (d) a heavy chain variable domain comprising the amino acid sequence of SEQ ID NO: 150 and a light chain variable domain comprising the amino acid sequence of SEQ ID NO: 151; or (e) a heavy chain variable domain comprising the amino acid sequence of SEQ ID NO: 166 and a light chain variable domain comprising the amino acid sequence of SEQ ID NO: 167. In some embodiments, the antigen binding site that specifically binds a tumor target protein comprises six CDRs, or a heavy chain and a light chain variable domain, shown in Tables 2-5. In some embodiments, the antigen binding site that specifically binds a tumor target protein comprises six CDRs, or a heavy chain and a light chain variable domain, of an anti-Her2, anti-CD19, anti-CD20, anti-CD38, or anti-LAMP1 binding domain shown in Tables 2-5.

In another embodiment, the disclosure provides a binding protein comprising four polypeptide chains that form three antigen binding sites, wherein a first polypeptide chain has a structure represented by the formula:

V_{L2}-L₁-V_{L1}-L₂-C_{L} [I]

and a second polypeptide chain has a structure represented by the formula:

V_{H1}-L₃-V_{H2}-L₄-C_{H1} [II]

and a third polypeptide chain has a structure represented by the formula:

V_{H3}-C_{H1} [III]

and a fourth polypeptide chain has a structure represented by the formula:

V_{L3}-C_{L} [IV]

wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain; and
L₁, L₂, L₃ and L₄ are amino acid linkers;
wherein the polypeptide of formula I and the polypeptide of formula II form a cross-over light chain-heavy chain pair;
wherein:
(a) V_{L1}, V_{L2} and V_{L3} are each independently a variable domain derived from an amino acid sequence as set forth in any one of SEQ ID NOs: 61, 63, 69, 71, 74, 76, 82, 86, 88, 94; or
(b) V_{L1}, V_{L2} and V_{L3} each independently comprise light chain complementarity determining regions of a variable domain of at least one amino acid sequence set forth in any one of SEQ ID NOs: 61, 63, 69, 71, 74, 76, 82, 86, 88, 94;
(c) V_{L1}, V_{L2} and V_{L3} each independently comprise a variable domain sequence as set forth in any one of SEQ ID NOs:169, 171, and 173;
(d) V_{L1}, V_{L2} and V_{L3} each independently comprise light chain complementarity determining regions comprising an amino acid sequence selected from the group consisting of SEQ ID NOs: 141-147, 178, and 179; or
(e) V_{L1}, V_{L2} and V_{L3} each independently comprise light chain complementarity determining regions and/or a variable domain sequence shown in Tables 2-5; and
wherein:
(a) V_{H1}, V_{H2}, and V_{H3} are each independently a variable domain derived from an amino acid sequence as set forth in any one of SEQ ID NOs: 60, 62, 68, 73, 75, 81, 85, 87, 93; or
(b) V_{H1}, V_{H2}, and V_{H3} each independently comprise heavy chain complementarity determining regions of a variable domain of at least one amino acid sequence set forth in any one of in any one of SEQ ID NOs: 60, 62, 68, 73, 75, 81, 85, 87, 93;
(c) V_{H1}, V_{H2}, and V_{H3} each independently comprise a variable domain sequence as set forth in any one of SEQ ID NOs:168, 170, and 172;
(d) V_{H1}, V_{H2} and V_{H3} each independently comprise heavy chain complementarity determining regions comprising an amino acid sequence selected from the group consisting of SEQ ID NOs: 129-137; or
(e) V_{H1}, V_{H2} and V_{H3} each independently comprise heavy chain complementarity determining regions and/or a variable domain sequence shown in Tables 2-5.

In some embodiments, the second and/or third polypeptide chain further comprises an Fc region linked to C_{H1}, the Fc region comprising an immunoglobulin hinge region and C_{H2} and C_{H3} immunoglobulin heavy chain constant domains.

In another embodiment, the disclosure provides a binding protein comprising four polypeptide chains that form three antigen binding sites, wherein a first polypeptide chain comprises a structure represented by the formula:

V_{L2}-L₁-V_{L1}-L₂-C_{L} [I]

and a second polypeptide chain comprises a structure represented by the formula:

V_{H1}-L₃-V_{H2}-L₄-C_{H1}-hinge-C_{H2}-C_{H3} [II]

and a third polypeptide chain comprises a structure represented by the formula:

V_{H3}-C_{H1} [III]

and a fourth polypeptide chain comprises a structure represented by the formula:

V_{L3}-C_{L} [IV]

wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain;
C_{H2} is an immunoglobulin C_{H2} heavy chain constant domain;
C_{H3} is an immunoglobulin C_{H3} heavy chain constant domain;
hinge is an immunoglobulin hinge region connecting the C_{H1} and C_{H2} domains; and
L₁, L₂, L₃ and L₄ are amino acid linkers;
wherein the polypeptide of formula I and the polypeptide of formula II form a cross-over light chain-heavy chain pair;
wherein:
(a) V_{L1}, V_{L2} and V_{L3} are each independently a variable domain derived from an amino acid sequence as set forth in any one of SEQ ID NOs: 61, 63, 69, 71, 74, 76, 82, 86, 88, 94;
(b) V_{L1}, V_{L2} and V_{L3} each independently comprise light chain complementarity determining regions of a variable domain of at least one amino acid sequence set forth in any one of SEQ ID NOs: 61, 63, 69, 71, 74, 76, 82, 86, 88, 94;
(c) V_{L1}, V_{L2} and V_{L3} each independently comprise a variable domain sequence as set forth in any one of SEQ ID NOs:169, 171, and 173;
(d) V_{L1}, V_{L2} and V_{L3} each independently comprise light chain complementarity determining regions comprising an amino acid sequence selected from the group consisting of SEQ ID NOs: 141-147, 178, and 179; or
(e) V_{L1}, V_{L2} and V_{L3} each independently comprise light chain complementarity determining regions and/or a variable domain sequence shown in Tables 2-5;
wherein:
(a) V_{H1}, V_{H2}, and V_{H3} are each independently a variable domain derived from an amino acid sequence as set forth in any one of SEQ ID NOs: 60, 62, 68, 73, 75, 81, 85, 87, 93;
(b) V_{H1}, V_{H2}, and V_{H3} each independently comprise heavy chain complementarity determining regions of a variable domain of at least one amino acid sequence set forth in any one of in any one of SEQ ID NOs: 60, 62, 68, 73, 75, 81, 85, 87, 93;
(c) V_{H1}, V_{H2}, and V_{H3} each independently comprise a variable domain sequence as set forth in any one of SEQ ID NOs:168, 170, and 172;
(d) V_{H1}, V_{H2} and V_{H3} each independently comprise heavy chain complementarity determining regions comprising an amino acid sequence selected from the group consisting of SEQ ID NOs: 129-137;
(e) V_{H1}, V_{H2} and V_{H3} each independently comprise heavy chain complementarity determining regions and/or a variable domain sequence shown in Tables 2-5.

In some embodiments, V_{L1}, V_{L2} and V_{L3} each independently comprise a variable domain sequence as set forth in any one of SEQ ID NOs:169, 171, and 173; and V_{H1}, V_{H2}, and V_{H3} each independently comprise a variable domain sequence as set forth in any one of SEQ ID NOs:168, 170, and 172. In some embodiments, V_{L1}, V_{L2} and V_{L3} each independently comprise light chain complementarity determining regions comprising an amino acid sequence selected from the group consisting of SEQ ID NOs: 141-147, 178, and 179; and V_{H1}, V_{H2} and V_{H3} each independently comprise heavy chain complementarity determining regions comprising an amino acid sequence selected from the group consisting of SEQ ID NOs: 129-137.

In some embodiments of any of the binding proteins described herein, (a) V_{H1} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; V_{L1} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144; V_{H2} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; V_{L2} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; V_{H3} comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131; and V_{L3} comprises a CDR-L1 comprising the amino acid sequence of SEQ ID NO:141, a CDR-L2 comprising the amino acid sequence of SEQ ID NO:178, and a CDR-L3 comprising the amino acid sequence of SEQ ID NO:142; (b) V_{H1} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; V_{L1} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; V_{H2} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; V_{L2} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144; V_{H3} comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131; and V_{L3} comprises a CDR-L1 comprising the amino acid sequence of SEQ ID NO:141, a CDR-L2 comprising the amino acid sequence of SEQ ID NO:178, and a CDR-L3 comprising the amino acid sequence of SEQ ID NO:142; (c) V_{H1} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; V_{L1} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144; V_{H2} comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131; V_{L2} comprises a CDR-L1 comprising the amino acid sequence of SEQ ID NO:141, a CDR-L2 comprising the amino acid sequence of SEQ ID NO:178, and a CDR-L3 comprising the amino acid sequence of SEQ ID NO:142; V_{H3} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; and V_{L3} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; (d) V_{H1} comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131; V_{L1} comprises a CDR-L1 comprising the amino acid sequence of SEQ ID NO:141, a CDR-L2 comprising the amino acid sequence of SEQ ID NO:178, and a CDR-L3 comprising the amino acid sequence of SEQ ID NO:142; V_{H2} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; V_{L2} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144; V_{H3} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; and V_{L3} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; (e) V_{H1} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; V_{L1} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; V_{H2} comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131; V_{L2} comprises a CDR-L1 comprising the amino acid sequence of SEQ ID NO:141, a CDR-L2 comprising the amino acid sequence of SEQ ID NO:178, and a CDR-L3 comprising the amino acid sequence of SEQ ID NO:142; V_{H3} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; and V_{L3} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144; (f) V_{H1} comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131; V_{L1} comprises a CDR-L1 comprising the amino acid sequence of SEQ ID NO:141, a CDR-L2 comprising the amino acid sequence of SEQ ID NO:178, and a CDR-L3 comprising the amino acid sequence of SEQ ID NO:142; V_{H2} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; V_{L2} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; V_{H3} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; and V_{L3} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144; (g) V_{H1} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; V_{L1} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; V_{H2} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; V_{L2} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144; V_{H3} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; and V_{L3} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; (h) V_{H1} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; V_{L1} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; V_{H2} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; V_{L2} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144; V_{H3} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; and V_{L3} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144; (i) V_{H1} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; V_{L1} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO: 144; V_{H2} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; V_{L2} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; V_{H3} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; and V_{L3} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; or (j) V_{H1} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; V_{L1} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144; V_{H2} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; V_{L2} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; V_{H3} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; and V_{L3} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144. In some embodiments, one or more of V_{H1}, V_{L1}, V_{H2}, V_{L2}, V_{H3}, and V_{L3} comprises one, two, or three CDR sequences of an antibody shown in Tables 2-5.

In some embodiments, the binding protein comprises three antigen binding sites, where one, two, or three of the antigen binding site(s) specifically bind(s) a cytokine target protein selected from the group consisting of IL-4, IL-13 and TNFa. In some embodiments, (a) V_{H1} and V_{L1} form a first antigen binding site that specifically binds human TNFa, V_{H2} and V_{L2} form an antigen binding site that specifically binds human IL13, and V_{H3} and V_{L3} form an antigen binding site that specifically binds human IL4; (b) V_{H1} and V_{L1} form a first antigen binding site that specifically binds human TNFa, V_{H2} and V_{L2} form a second antigen binding site that specifically binds human IL4, and V_{H3} and V_{L3} form a third antigen binding site that specifically binds human IL13; (c) V_{H1} and V_{L1} form a first antigen binding site that specifically binds human IL4, V_{H2} and V_{L2} form a second antigen binding site that specifically binds human TNFa, and V_{H3} and V_{L3} form a third antigen binding site that specifically binds human IL13; (d) V_{H1} and V_{L1} form a first antigen binding site that specifically binds human IL4, V_{H2} and V_{L2} form a second antigen binding site that specifically binds human IL13, and V_{H3} and V_{L3} form a third antigen binding site that specifically binds human TNFa; (e) V_{H1} and V_{L1} form a first antigen binding site that specifically binds human IL13, V_{H2} and V_{L2} form a second antigen binding site that specifically binds human IL4, and V_{H3} and V_{L3} form a third antigen binding site that specifically binds human TNFa; or (f) V_{H1} and V_{L1} form a first antigen binding site that specifically binds human IL13, V_{H2} and V_{L2} form a second antigen binding site that specifically binds human TNFa, and V_{H3} and V_{L3} form a third antigen binding site that specifically binds human IL4. In some embodiments, the antigen binding site that specifically binds human TNFa comprises a heavy chain variable domain comprising the amino acid sequence of SEQ ID NO:168 and a light chain variable domain comprising the amino acid sequence of SEQ ID NO:169. In some embodiments, the antigen binding site that specifically binds human IL4 comprises a heavy chain variable domain comprising the amino acid sequence of SEQ ID NO:170 and a light chain variable domain comprising the amino acid sequence of SEQ ID NO:171. In some embodiments, the antigen binding site that specifically binds human IL13 comprises a heavy chain variable domain comprising the amino acid sequence of SEQ ID NO:172 and a light chain variable domain comprising the amino acid sequence of SEQ ID NO:173.

In some embodiments of any of the binding proteins described herein, the second and/or third polypeptide chain further comprises an Fc region linked to C_{H1}, the Fc region comprising an immunoglobulin hinge region and C_{H2} and C_{H3} immunoglobulin heavy chain constant domains. In some embodiments, at least one of L₁, L₂, L₃ or L₄ is independently 0 amino acids in length. In some embodiments, L₁, L₂, L₃ or L₄ are each independently at least one amino acid in length. In some embodiments, the binding protein is trispecific and capable of specifically binding three different antigen targets. In some embodiments, the binding protein is trispecific and capable of specifically binding three different antigen targets. In some embodiments, the binding protein is capable of inhibiting the function of one or more target proteins.

In some embodiments of any of the binding proteins described herein, at least one of L₁, L₂, L₃ or L₄ is independently 0 amino acids in length. In some embodiments, L₁, L₂, L₃ or L₄ are each independently at least one amino acid in length. In some embodiments, one, two, three, or all four of L₁, L₂, L₃ and L₄ are between 0 and 15 amino acids in length. In some embodiments, at least two of L₁, L₂, L₃ and L₄ are between 1 and 15 amino acids in length. In some embodiments, (a) L₁, L₂, L₃ and L₄ each independently are zero amino acids in length or comprise a sequence selected from the group consisting of GGGGSGGGGS (SEQ ID NO:104), GGGGSGGGGSGGGGS (SEQ ID NO:105), S, RT, TKGPS (SEQ ID NO:106), GQPKAAP (SEQ ID NO: 175), and GGSGSSGSGG (SEQ ID NO:148); or (b) L₁, L₂, L₃ and L₄ each independently comprise a sequence selected from the group consisting of GGGGSGGGGS (SEQ ID NO:104), GGGGSGGGGSGGGGS (SEQ ID NO:105), S, RT, TKGPS (SEQ ID NO:106), GQPKAAP (SEQ ID NO: 175), and GGSGSSGSGG (SEQ ID NO:148). In some embodiments, L₁ comprises the sequence GQPKAAP (SEQ ID NO: 175), L₂ comprises the sequence TKGPS (SEQ ID NO:106), L₃ comprises the sequence S, and L₄ comprises the sequence RT; L₁ comprises the sequence GGGGSGGGGS (SEQ ID NO:104), L₂ comprises the sequence GGGGSGGGGS (SEQ ID NO:104), L₃ is 0 amino acids in length, and L₄ is 0 amino acids in length; L₁ comprises the sequence GGSGSSGSGG (SEQ ID NO:148), L₂ comprises the sequence GGSGSSGSGG (SEQ ID NO:148), L₃ is 0 amino acids in length, and L₄ is 0 amino acids in length; or L₁ comprises the sequence GGGGSGGGGSGGGGS (SEQ ID NO:105), L₂ is 0 amino acids in length, L₃ comprises the sequence GGGGSGGGGSGGGGS (SEQ ID NO:105), and L₄ is 0 amino acids in length.

In some embodiments of any of the binding proteins described herein, the second polypeptide chain further comprises a first Fc region linked to C_{H1}, the first Fc region comprising an immunoglobulin hinge region and C_{H2} and C_{H3} immunoglobulin heavy chain constant domains, wherein the first Fc region comprises amino acid substitutions at positions corresponding to positions 354 and 366 of human IgG1 or IgG4 according to EU Index, wherein the amino acid substitutions are S354C and T366W; and wherein the third polypeptide chain further comprises a second Fc region linked to C_{H1}, the second Fc region comprising an immunoglobulin hinge region and C_{H2} and C_{H3} immunoglobulin heavy chain constant domains, wherein the second Fc region comprises amino acid substitutions at positions corresponding to positions 349, 366, 368, and 407 of human IgG1 or IgG4 according to EU Index, wherein the amino acid substitutions are Y349C, T366S, L368A, and Y407V. In some embodiments, the second polypeptide chain further comprises a first Fc region linked to C_{H1}, the first Fc region comprising an immunoglobulin hinge region and C_{H2} and C_{H3} immunoglobulin heavy chain constant domains, wherein the first Fc region comprises amino acid substitutions at positions corresponding to positions 349, 366, 368, and 407 of human IgG1 or IgG4 according to EU Index, wherein the amino acid substitutions are Y349C, T366S, L368A, and Y407V; and wherein the third polypeptide chain further comprises a second Fc region linked to C_{H1}, the second Fc region comprising an immunoglobulin hinge region and C_{H2} and C_{H3} immunoglobulin heavy chain constant domains, wherein the second Fc region comprises amino acid substitutions at positions corresponding to positions 354 and 366 of human IgG1 or IgG4 according to EU Index, wherein the amino acid substitutions are S354C and T366W. In some embodiments, the second polypeptide chain further comprises a first Fc region linked to C_{H1}, the first Fc region comprising an immunoglobulin hinge region and C_{H2} and C_{H3} immunoglobulin heavy chain constant domains, and wherein the third polypeptide chain further comprises a second Fc region linked to C_{H1}, the second Fc region comprising an immunoglobulin hinge region and C_{H2} and C_{H3} immunoglobulin heavy chain constant domains; wherein the first and/or second Fc regions comprise amino acid substitutions at positions corresponding to positions 428 and 434 of human IgG1 or IgG4 according to EU Index, wherein the amino acid substitutions are M428L and N434S. In some embodiments, the C_{H3} domain of the second polypeptide chain comprises amino acid substitutions at positions corresponding to positions 354 and 366 of human IgG1 or IgG4 according to EU Index, wherein the amino acid substitutions are S354C and T366W; and wherein the C_{H3} domain of the third polypeptide chain comprises amino acid substitutions at positions corresponding to positions 349, 366, 368, and 407 of human IgG1 or IgG4 according to EU Index, wherein the amino acid substitutions are Y349C, T366S, L368A, and Y407V. In some embodiments, the C_{H3} domain of the second polypeptide chain comprises amino acid substitutions at positions corresponding to positions 349, 366, 368, and 407 of human IgG1 or IgG4 according to EU Index, wherein the amino acid substitutions are Y349C, T366S, L368A, and Y407V; and wherein the C_{H3} domain of the third polypeptide chain comprises amino acid substitutions at positions corresponding to positions 354 and 366 of human IgG1 or IgG4 according to EU Index, wherein the amino acid substitutions are S354C and T366W. In some embodiments, the C_{H3} domains of the second and the third polypeptide chains both comprise amino acid substitutions at positions corresponding to positions 428 and 434 of human IgG1 or IgG4 according to EU Index, wherein the amino acid substitutions are M428L and N434S. In some embodiments, the second polypeptide chain further comprises a first Fc region linked to C_{H1}, the first Fc region comprising an immunoglobulin hinge region and C_{H2} and C_{H3} immunoglobulin heavy chain constant domains; wherein the third polypeptide chain further comprises a second Fc region linked to C_{H1}, the second Fc region comprising an immunoglobulin hinge region and C_{H2} and C_{H3} immunoglobulin heavy chain constant domains; and wherein only one of the first and the second Fc regions comprises amino acid substitutions at positions corresponding to positions 435 and 436 of human IgG1 or IgG4 according to EU Index, wherein the amino acid substitutions are H435R and Y436F. In some embodiments, the C_{H3} domains of the second and the third polypeptide chains are human IgG1 C_{H3} domains, and wherein only one of the C_{H3} domains comprises amino acid substitutions at positions corresponding to positions 435 and 436 of human IgG1 or IgG4 according to EU Index, wherein the amino acid substitutions are H435R and Y436F. In some embodiments, the second polypeptide chain further comprises a first Fc region linked to C_{H1}, the first Fc region comprising an immunoglobulin hinge region and C_{H2} and C_{H3} immunoglobulin heavy chain constant domains; wherein the third polypeptide chain further comprises a second Fc region linked to C_{H1}, the second Fc region comprising an immunoglobulin hinge region and C_{H2} and C_{H3} immunoglobulin heavy chain constant domains; wherein the first and/or second Fc regions are human IgG4 Fc regions; and wherein the first and the second Fc regions each comprise amino acid substitutions at positions corresponding to positions 228 and 409 of human IgG4 according to EU Index, wherein the amino acid substitutions are S228P and R409K. In some embodiments, the C_{H3} domains of the second and the third polypeptide chains are human IgG4 C_{H3} domains, and wherein the C_{H3} domains each comprise amino acid substitutions at positions corresponding to positions 228 and 409 of human IgG4 according to EU Index, wherein the amino acid substitutions are S228P and R409K. In some embodiments, the second polypeptide chain further comprises a first Fc region linked to C_{H1}, the first Fc region comprising an immunoglobulin hinge region and C_{H2} and C_{H3} immunoglobulin heavy chain constant domains; wherein the third polypeptide chain further comprises a second Fc region linked to C_{H1}, the second Fc region comprising an immunoglobulin hinge region and C_{H2} and C_{H3} immunoglobulin heavy chain constant domains; wherein the first and/or second Fc regions are human IgG4 Fc regions; and wherein the first and the second Fc regions each comprise amino acid substitutions at positions corresponding to positions 234 and 235 of human IgG4 according to EU Index, wherein the amino acid substitutions are F234A and L235A. In some embodiments, the C_{H3} domains of the second and the third polypeptide chains are human IgG4 C_{H3} domains, and wherein the C_{H3} domains each comprise amino acid substitutions at positions corresponding to positions 234 and 235 of human IgG4 according to EU Index, wherein the amino acid substitutions are F234A and L235A. In some embodiments, the second polypeptide chain further comprises a first Fc region linked to C_{H1}, the first Fc region comprising an immunoglobulin hinge region and C_{H2} and C_{H3} immunoglobulin heavy chain constant domains; wherein the third polypeptide chain further comprises a second Fc region linked to C_{H1}, the second Fc region comprising an immunoglobulin hinge region and C_{H2} and C_{H3} immunoglobulin heavy chain constant domains; wherein the first and/or second Fc regions are human IgG1 Fc regions; and wherein the first and the second Fc regions each comprise amino acid substitutions at positions corresponding to positions 234 and 235 of human IgG1 according to EU Index, wherein the amino acid substitutions are L234A and L235A. In some embodiments, the C_{H3} domains of the second and the third polypeptide chains are human IgG1 C_{H3} domains, and wherein the C_{H3} domains each comprise amino acid substitutions at positions corresponding to positions 234 and 235 of human IgG1 according to EU Index, wherein the amino acid substitutions are L234A and L235A. In some embodiments, the first and/or second Fc regions are human IgG1 Fc regions. In some embodiments, the first and/or second Fc regions are human IgG4 Fc regions.

In some embodiments of any of the binding proteins described herein, the C_{L} domain of the first polypeptide chain is a human kappa C_{L} domain, and the C_{L} domain of the fourth polypeptide chain is a human lambda C_{L} domain; or the C_{L} domain of the first polypeptide chain is a human lambda C_{L} domain, and the C_{L} domain of the fourth polypeptide chain is a human kappa C_{L} domain. In some embodiments, the first polypeptide chain comprises a lambda C_{L} domain; wherein the C_{H3} domain of the second polypeptide chain comprises amino acid substitutions at positions corresponding to positions 354 and 366 of human IgG1 or IgG4 according to EU Index, wherein the amino acid substitutions are S354C and T366W; wherein the C_{H3} domain of the third polypeptide chain comprises amino acid substitutions at positions corresponding to positions 349, 366, 368, 407, 435, and 436 of human IgG1 or IgG4 according to EU Index, wherein the amino acid substitutions are Y349C, T366S, L368A, Y407V, H435R, and Y436F; and wherein the fourth polypeptide chain comprises a kappa C_{L} domain. In some embodiments, the first polypeptide chain comprises a lambda C_{L} domain; wherein the C_{H3} domain of the third polypeptide chain comprises amino acid substitutions at positions corresponding to positions 354 and 366 of human IgG1 or IgG4 according to EU Index, wherein the amino acid substitutions are S354C and T366W; wherein the C_{H3} domain of the second polypeptide chain comprises amino acid substitutions at positions corresponding to positions 349, 366, 368, 407, 435, and 436 of human IgG1 or IgG4 according to EU Index, wherein the amino acid substitutions are Y349C, T366S, L368A, Y407V, H435R, and Y436F; and wherein the fourth polypeptide chain comprises a kappa C_{L} domain. In some embodiments, the first polypeptide chain comprises a lambda C_{L} domain; wherein the C_{H3} domain of the second polypeptide chain comprises amino acid substitutions at positions corresponding to positions 354, 366, 435, and 436 of human IgG1 or IgG4 according to EU Index, wherein the amino acid substitutions are S354C, T366W, H435R, and Y436F; wherein the C_{H3} domain of the third polypeptide chain comprises amino acid substitutions at positions corresponding to positions 349, 366, 368, and 407 of human IgG1 or IgG4 according to EU Index, wherein the amino acid substitutions are Y349C, T366S, L368A, and Y407V; and wherein the fourth polypeptide chain comprises a kappa C_{L} domain. In some embodiments, the first polypeptide chain comprises a kappa C_{L} domain; wherein the C_{H3} domain of the second polypeptide chain comprises amino acid substitutions at positions corresponding to positions 354 and 366 of human IgG1 or IgG4 according to EU Index, wherein the amino acid substitutions are S354C and T366W; wherein the C_{H3} domain of the third polypeptide chain comprises amino acid substitutions at positions corresponding to positions 349, 366, 368, 407, 435, and 436 of human IgG1 or IgG4 according to EU Index, wherein the amino acid substitutions are Y349C, T366S, L368A, Y407V, H435R, and Y436F; and wherein the fourth polypeptide chain comprises a lambda C_{L} domain. In some embodiments, second and/or third polypeptide chain comprise a human IgG1 or IgG4 Fc region.

In another embodiment, the disclosure provides a binding protein comprising a first polypeptide chain, a second polypeptide chain, a third polypeptide chain and a fourth polypeptide chain wherein:
(a) the first polypeptide chain comprises the amino acid sequence of SEQ ID NO: 4 or an amino acid sequence that is at least 95% identical to the amino acid sequence of SEQ ID NO: 4; the second polypeptide chain comprises the amino acid sequence of SEQ ID NO: 3 or an amino acid sequence that is at least 95% identical to the amino acid sequence of SEQ ID NO: 3; the third polypeptide chain comprises the amino acid sequence of SEQ ID NO: 1 or an amino acid sequence that is at least 95% identical to the amino acid sequence of SEQ ID NO: 1; and the fourth polypeptide chain comprises the amino acid sequence of SEQ ID NO: 2 or an amino acid sequence that is at least 95% identical to the amino acid sequence of SEQ ID NO: 2;
(b) the first polypeptide chain comprises the amino acid sequence of SEQ ID NO: 10 or an amino acid sequence that is at least 95% identical to the amino acid sequence of SEQ ID NO: 10; the second polypeptide chain comprises the amino acid sequence of SEQ ID NO: 9 or an amino acid sequence that is at least 95% identical to the amino acid sequence of SEQ ID NO: 9; the third polypeptide chain comprises the amino acid sequence of SEQ ID NO: 1 or an amino acid sequence that is at least 95% identical to the amino acid sequence of SEQ ID NO: 1; and the fourth polypeptide chain comprises the amino acid sequence of SEQ ID NO: 2 or an amino acid sequence that is at least 95% identical to the amino acid sequence of SEQ ID NO: 2;
(c) the first polypeptide chain comprises the amino acid sequence of SEQ ID NO: 4 or an amino acid sequence that is at least 95% identical to the amino acid sequence of SEQ ID NO: 4; the second polypeptide chain comprises the amino acid sequence of SEQ ID NO: 3 or an amino acid sequence that is at least 95% identical to the amino acid sequence of SEQ ID NO: 3; the third polypeptide chain comprises the amino acid sequence of SEQ ID NO: 13 or an amino acid sequence that is at least 95% identical to the amino acid sequence of SEQ ID NO: 13; and the fourth polypeptide chain comprises the amino acid sequence of SEQ ID NO: 14 or an amino acid sequence that is at least 95% identical to the amino acid sequence of SEQ ID NO: 14;
(d) the first polypeptide chain comprises the amino acid sequence of SEQ ID NO: 10 or an amino acid sequence that is at least 95% identical to the amino acid sequence of SEQ ID NO: 10; the second polypeptide chain comprises the amino acid sequence of SEQ ID NO: 9 or an amino acid sequence that is at least 95% identical to the amino acid sequence of SEQ ID NO: 9; the third polypeptide chain comprises the amino acid sequence of SEQ ID NO: 13 or an amino acid sequence that is at least 95% identical to the amino acid sequence of SEQ ID NO: 13; and the fourth polypeptide chain comprises the amino acid sequence of SEQ ID NO: 14 or an amino acid sequence that is at least 95% identical to the amino acid sequence of SEQ ID NO: 14;
(e) the first polypeptide chain comprises the amino acid sequence of SEQ ID NO: 4 or an amino acid sequence that is at least 95% identical to the amino acid sequence of SEQ ID NO: 4; the second polypeptide chain comprises the amino acid sequence of SEQ ID NO: 3 or an amino acid sequence that is at least 95% identical to the amino acid sequence of SEQ ID NO: 3; the third polypeptide chain comprises the amino acid sequence of SEQ ID NO: 17 or an amino acid sequence that is at least 95% identical to the amino acid sequence of SEQ ID NO: 17; and the fourth polypeptide chain comprises the amino acid sequence of SEQ ID NO: 18 or an amino acid sequence that is at least 95% identical to the amino acid sequence of SEQ ID NO: 18
(f) the first polypeptide chain comprises the amino acid sequence of SEQ ID NO: 10 or an amino acid sequence that is at least 95% identical to the amino acid sequence of SEQ ID NO: 10; the second polypeptide chain comprises the amino acid sequence of SEQ ID NO: 9 or an amino acid sequence that is at least 95% identical to the amino acid sequence of SEQ ID NO: 9; the third polypeptide chain comprises the amino acid sequence of SEQ ID NO: 17 or an amino acid sequence that is at least 95% identical to the amino acid sequence of SEQ ID NO: 17; and the fourth polypeptide chain comprises the amino acid sequence of SEQ ID NO: 18 or an amino acid sequence that is at least 95% identical to the amino acid sequence of SEQ ID NO: 18;
(g) the first polypeptide chain comprises the amino acid sequence of SEQ ID NO: 4 or an amino acid sequence that is at least 95% identical to the amino acid sequence of SEQ ID NO: 4; the second polypeptide chain comprises the amino acid sequence of SEQ ID NO: 3 or an amino acid sequence that is at least 95% identical to the amino acid sequence of SEQ ID NO: 3; the third polypeptide chain comprises the amino acid sequence of SEQ ID NO: 21 or an amino acid sequence that is at least 95% identical to the amino acid sequence of SEQ ID NO: 21; and the fourth polypeptide chain comprises the amino acid sequence of SEQ ID NO: 22 or an amino acid sequence that is at least 95% identical to the amino acid sequence of SEQ ID NO: 22;
(h) the first polypeptide chain comprises the amino acid sequence of SEQ ID NO: 10 or an amino acid sequence that is at least 95% identical to the amino acid sequence of SEQ ID NO: 10; the second polypeptide chain comprises the amino acid sequence of SEQ ID NO: 9 or an amino acid sequence that is at least 95% identical to the amino acid sequence of SEQ ID NO: 9; the third polypeptide chain comprises the amino acid sequence of SEQ ID NO: 21 or an amino acid sequence that is at least 95% identical to the amino acid sequence of SEQ ID NO: 21; and the fourth polypeptide chain comprises the amino acid sequence of SEQ ID NO: 22 or an amino acid sequence that is at least 95% identical to the amino acid sequence of SEQ ID NO: 22;
(i) the first polypeptide chain comprises the amino acid sequence of SEQ ID NO: 63 or an amino acid sequence that is at least 95% identical to the amino acid sequence of SEQ ID NO: 63; the second polypeptide chain comprises the amino acid sequence of SEQ ID NO: 62 or an amino acid sequence that is at least 95% identical to the amino acid sequence of SEQ ID NO: 62; the third polypeptide chain comprises the amino acid sequence of SEQ ID NO: 60 or an amino acid sequence that is at least 95% identical to the amino acid sequence of SEQ ID NO: 60; and the fourth polypeptide chain comprises the amino acid sequence of SEQ ID NO: 61 or an amino acid sequence that is at least 95% identical to the amino acid sequence of SEQ ID NO: 61;
(j) the first polypeptide chain comprises the amino acid sequence of SEQ ID NO: 69 or an amino acid sequence that is at least 95% identical to the amino acid sequence of SEQ ID NO: 69; the second polypeptide chain comprises the amino acid sequence of SEQ ID NO: 68 or an amino acid sequence that is at least 95% identical to the amino acid sequence of SEQ ID NO: 68; the third polypeptide chain comprises the amino acid sequence of SEQ ID NO: 60 or an amino acid sequence that is at least 95% identical to the amino acid sequence of SEQ ID NO: 60; and the fourth polypeptide chain comprises the amino acid sequence of SEQ ID NO: 61 or an amino acid sequence that is at least 95% identical to the amino acid sequence of SEQ ID NO: 61;
(k) the first polypeptide chain comprises the amino acid sequence of SEQ ID NO: 69 or an amino acid sequence that is at least 95% identical to the amino acid sequence of SEQ ID NO: 69; the second polypeptide chain comprises the amino acid sequence of SEQ ID NO: 68 or an amino acid sequence that is at least 95% identical to the amino acid sequence of SEQ ID NO: 68; the third polypeptide chain comprises the amino acid sequence of SEQ ID NO: 60 or an amino acid sequence that is at least 95% identical to the amino acid sequence of SEQ ID NO: 60; and the fourth polypeptide chain comprises the amino acid sequence of SEQ ID NO: 71 or an amino acid sequence that is at least 95% identical to the amino acid sequence of SEQ ID NO: 71; (l) the first polypeptide chain comprises the amino acid sequence of SEQ ID NO: 76 or an amino acid sequence that is at least 95% identical to the amino acid sequence of SEQ ID NO: 76; the second polypeptide chain comprises the amino acid sequence of SEQ ID NO: 75 or an amino acid sequence that is at least 95% identical to the amino acid sequence of SEQ ID NO: 75; the third polypeptide chain comprises the amino acid sequence of SEQ ID NO: 73 or an amino acid sequence that is at least 95% identical to the amino acid sequence of SEQ ID NO: 73; and the fourth polypeptide chain comprises the amino acid sequence of SEQ ID NO: 74 or an amino acid sequence that is at least 95% identical to the amino acid sequence of SEQ ID NO: 74;
(m) the first polypeptide chain comprises the amino acid sequence of SEQ ID NO: 82 or an amino acid sequence that is at least 95% identical to the amino acid sequence of SEQ ID NO: 82; the second polypeptide chain comprises the amino acid sequence of SEQ ID NO: 81 or an amino acid sequence that is at least 95% identical to the amino acid sequence of SEQ ID NO:81; the third polypeptide chain comprises the amino acid sequence of SEQ ID NO: 73 or an amino acid sequence that is at least 95% identical to the amino acid sequence of SEQ ID NO: 73; and the fourth polypeptide chain comprises the amino acid sequence of SEQ ID NO: 74 or an amino acid sequence that is at least 95% identical to the amino acid sequence of SEQ ID NO: 74;
(n) the first polypeptide chain comprises the amino acid sequence of SEQ ID NO: 88 or an amino acid sequence that is at least 95% identical to the amino acid sequence of SEQ ID NO:88; the second polypeptide chain comprises the amino acid sequence of SEQ ID NO: 87 or an amino acid sequence that is at least 95% identical to the amino acid sequence of SEQ ID NO: 87; the third polypeptide chain comprises the amino acid sequence of SEQ ID NO: 85 or an amino acid sequence that is at least 95% identical to the amino acid sequence of SEQ ID NO: 85; and the fourth polypeptide chain comprises the amino acid sequence of SEQ ID NO: 86 or an amino acid sequence that is at least 95% identical to the amino acid sequence of SEQ ID NO: 86;
(o) the first polypeptide chain comprises the amino acid sequence of SEQ ID NO: 94 or an amino acid sequence that is at least 95% identical to the amino acid sequence of SEQ ID NO: 94; the second polypeptide chain comprises the amino acid sequence of SEQ ID NO: 93 or an amino acid sequence that is at least 95% identical to the amino acid sequence of SEQ ID NO: 93; the third polypeptide chain comprises the amino acid sequence of SEQ ID NO: 85 or an amino acid sequence that is at least 95% identical to the amino acid sequence of SEQ ID NO: 85; and the fourth polypeptide chain comprises the amino acid sequence of SEQ ID NO: 86 or an amino acid sequence that is at least 95% identical to the amino acid sequence of SEQ ID NO: 86;
(p) the first polypeptide chain comprises the amino acid sequence of SEQ ID NO: 69 or an amino acid sequence that is at least 95% identical to the amino acid sequence of SEQ ID NO: 69; the second polypeptide chain comprises the amino acid sequence of SEQ ID NO: 68 or an amino acid sequence that is at least 95% identical to the amino acid sequence of SEQ ID NO: 68; the third polypeptide chain comprises the amino acid sequence of SEQ ID NO: 73 or an amino acid sequence that is at least 95% identical to the amino acid sequence of SEQ ID NO: 73; and the fourth polypeptide chain comprises the amino acid sequence of SEQ ID NO: 74 or an amino acid sequence that is at least 95% identical to the amino acid sequence of SEQ ID NO: 74;
(q) the first polypeptide chain comprises the amino acid sequence of SEQ ID NO: 69 or an amino acid sequence that is at least 95% identical to the amino acid sequence of SEQ ID NO: 69; the second polypeptide chain comprises the amino acid sequence of SEQ ID NO: 68 or an amino acid sequence that is at least 95% identical to the amino acid sequence of SEQ ID NO: 68; the third polypeptide chain comprises the amino acid sequence of SEQ ID NO: 85 or an amino acid sequence that is at least 95% identical to the amino acid sequence of SEQ ID NO: 85; and the fourth polypeptide chain comprises the amino acid sequence of SEQ ID NO: 86 or an amino acid sequence that is at least 95% identical to the amino acid sequence of SEQ ID NO: 86;
(r) the first polypeptide chain comprises the amino acid sequence of SEQ ID NO: 63 or an amino acid sequence that is at least 95% identical to the amino acid sequence of SEQ ID NO: 63; the second polypeptide chain comprises the amino acid sequence of SEQ ID NO: 62 or an amino acid sequence that is at least 95% identical to the amino acid sequence of SEQ ID NO: 62; the third polypeptide chain comprises the amino acid sequence of SEQ ID NO: 73 or an amino acid sequence that is at least 95% identical to the amino acid sequence of SEQ ID NO: 73; and the fourth polypeptide chain comprises the amino acid sequence of SEQ ID NO: 74 or an amino acid sequence that is at least 95% identical to the amino acid sequence of SEQ ID NO: 74;
(s) the first polypeptide chain comprises the amino acid sequence of SEQ ID NO: 63 or an amino acid sequence that is at least 95% identical to the amino acid sequence of SEQ ID NO: 63; the second polypeptide chain comprises the amino acid sequence of SEQ ID NO: 62 or an amino acid sequence that is at least 95% identical to the amino acid sequence of SEQ ID NO: 62; the third polypeptide chain comprises the amino acid sequence of SEQ ID NO: 85 or an amino acid sequence that is at least 95% identical to the amino acid sequence of SEQ ID NO: 85; and the fourth polypeptide chain comprises the amino acid sequence of SEQ ID NO: 86 or an amino acid sequence that is at least 95% identical to the amino acid sequence of SEQ ID NO: 86;
(t) the first polypeptide chain comprises the amino acid sequence of SEQ ID NO: 4 or an amino acid sequence that is at least 95% identical to the amino acid sequence of SEQ ID NO: 4; the second polypeptide chain comprises the amino acid sequence of SEQ ID NO: 3 or an amino acid sequence that is at least 95% identical to the amino acid sequence of SEQ ID NO: 3; the third polypeptide chain comprises the amino acid sequence of SEQ ID NO: 114 or an amino acid sequence that is at least 95% identical to the amino acid sequence of SEQ ID NO: 114; and the fourth polypeptide chain comprises the amino acid sequence of SEQ ID NO: 115 or an amino acid sequence that is at least 95% identical to the amino acid sequence of SEQ ID NO: 115; or
(u) the first polypeptide chain comprises the amino acid sequence of SEQ ID NO: 10 or an amino acid sequence that is at least 95% identical to the amino acid sequence of SEQ ID NO: 10; the second polypeptide chain comprises the amino acid sequence of SEQ ID NO: 9 or an amino acid sequence that is at least 95% identical to the amino acid sequence of SEQ ID NO: 9; the third polypeptide chain comprises the amino acid sequence of SEQ ID NO: 114 or an amino acid sequence that is at least 95% identical to the amino acid sequence of SEQ ID NO: 114; and the fourth polypeptide chain comprises the amino acid sequence of SEQ ID NO: 115 or an amino acid sequence that is at least 95% identical to the amino acid sequence of SEQ ID NO: 115.

In another embodiment, the disclosure provides an isolated nucleic acid molecule comprising a nucleotide sequence encoding the binding protein or polypeptide thereof according to any of the above embodiments. In another embodiment, the disclosure provides an expression vector comprising the nucleic acid molecule according to one of the above embodiments. In another embodiment, the disclosure provides an isolated host cell comprising the nucleic acid molecule according to any of the above embodiments. In another embodiment, the disclosure provides an isolated host cell comprising the expression vector according to any of the above embodiments. In some embodiments, the isolated host cell is a mammalian cell or an insect cell. In one embodiment, the disclosure provides a vector system comprising one or more vectors encoding a first, second, third, and fourth polypeptide chain of a binding protein according to any of the above embodiments. In some embodiments, the vector system comprises a first vector encoding the first polypeptide chain of the binding protein, a second vector encoding the second polypeptide chain of the binding protein, a third vector encoding the third polypeptide chain of the binding protein, and a fourth vector encoding the fourth polypeptide chain of the binding protein. In some embodiments, the vector system comprises a first vector encoding the first and second polypeptide chains of the binding protein, and a second vector encoding the third and fourth polypeptide chains of the binding protein. In some embodiments, the one or more vectors are expression vectors. In one embodiment, the disclosure provides an isolated host cell comprising the vector system according to any of the above embodiments. In one embodiment, the disclosure provides a method of producing a binding protein, the method comprising: a) culturing a host cell according to any of the above embodiments under conditions such that the host cell expresses the binding protein; and b) isolating the binding protein from the host cell. In one embodiment, the disclosure provides a pharmaceutical composition comprising the binding protein according to any of the above embodiments and a pharmaceutically acceptable carrier.

In another embodiment, the disclosure provides a method of preventing and/or treating cancer in a patient comprising administering to the patient a therapeutically effective amount of at least one binding protein or pharmaceutical composition according to any of the above embodiments. In another embodiment, the disclosure provides a binding protein or pharmaceutical composition according to any of the above embodiments for use in preventing and/or treating cancer in a patient. In another embodiment, the disclosure provides a binding protein according to any of the above embodiments for the manufacture of a medicament for preventing and/or treating cancer in a patient. In some embodiments, the binding protein comprises one antigen binding site that specifically binds a T-cell surface protein and another antigen binding site that specifically binds a tumor target protein. In some embodiments, the binding protein comprises an antigen binding site that specifically binds CD3, an antigen binding site that specifically binds CD28, and an antigen binding site that specifically binds a tumor target protein selected from the group consisting of CD19, CD20, CD38, Her2, and LAMP1. In some embodiments, the at least one binding protein is co-administered with a chemotherapeutic agent. In some embodiments, the patient is a human. In some embodiments, the binding protein is capable of inhibiting the function of one or more target proteins selected from the group consisting of A2AR, APRIL, ATPDase, BAFF, BAFFR, BCMA, BlyS, BTK, BTLA, B7DC, B7H1, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL24, CCL25, CCL26, CCR3, CCR4, CD3, CD19, CD20, CD23, CD24, CD27, CD28, CD38, CD39, CD40, CD70, CD80, CD86, CD122, CD137, CD137L, CD152, CD154, CD160, CD272, CD273, CD274, CD275, CD276, CD278, CD279, CDH1, chitinase, CLEC9, CLEC91, CRTH2, CSF-1, CSF-2, CSF-3, CX3CL1, CXCL12, CXCL13, CXCR3, DNGR-1, ectonucleoside triphosphate diphosphohydrolase 1, EGFR, ENTPD1, FCER1A, FCER1, FLAP, FOLH1, Gi24, GITR, GITRL, GM-CSF, Her2, HHLA2, HMGB1, HVEM, ICOSLG, IDO, IFNα, IgE, IGF1R, IL2Rbeta, IL1, IL1A, IL1B, IL1F10, IL2, IL4, IL4Ra, IL5, IL5R, IL6, IL7, IL7Ra, IL8, IL9, IL9R, IL10, rhIL10, IL12, IL13, IL13Ra1, IL13Ra2, IL15, IL17, IL17Rb, IL18, IL22, IL23, IL25, IL27, IL33, IL35, ITGB4, ITK, KIR, LAG3, LAMP1, leptin, LPFS2, MHC class II, NCR3LG1, NKG2D, NTPDase-1, OX40, OX40L, PD-1H, platelet receptor, PROM1, S152, SISP1, SLC, SPG64, ST2, STEAP2, Syk kinase, TACI, TDO, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, WUCAM, and XCR1.

In another embodiment, the disclosure provides a method of preventing and/or treating an inflammatory disease or disorder in a patient comprising administering to the patient a therapeutically effective amount of at least one binding protein or pharmaceutical composition according to any of the above embodiments. In another embodiment, the disclosure provides a binding protein or pharmaceutical composition according to any of the above embodiments for use in preventing and/or treating an inflammatory disease or disorder in a patient. In another embodiment, the disclosure provides a binding protein according to any of the above embodiments for the manufacture of a medicament for preventing and/or treating an inflammatory disease or disorder in a patient. In some embodiments, the binding protein comprises three antigen binding sites that each specifically bind a cytokine target protein selected from the group consisting of IL-4, IL-13 and TNFa. In some embodiments, two of the three binding sites specifically bind a cytokine target protein selected from the group consisting of IL-4, IL-13 and TNFa. In some embodiments, the at least one binding protein is co-administered with an anti-inflammatory agent. In some embodiments, the patient is a human. In some embodiments, the binding protein is capable of inhibiting the function of one or more target proteins selected from the group consisting of A2AR, APRIL, ATPDase, BAFF, BAFFR, BCMA, BlyS, BTK, BTLA, B7DC, B7H1, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL24, CCL25, CCL26, CCR3, CCR4, CD3, CD19, CD20, CD23, CD24, CD27, CD28, CD38, CD39, CD40, CD70, CD80, CD86, CD122, CD137, CD137L, CD152, CD154, CD160, CD272, CD273, CD274, CD275, CD276, CD278, CD279, CDH1, chitinase, CLEC9, CLEC91, CRTH2, CSF-1, CSF-2, CSF-3, CX3CL1, CXCL12, CXCL13, CXCR3, DNGR-1, ectonucleoside triphosphate diphosphohydrolase 1, EGFR, ENTPD1, FCER1A, FCER1, FLAP, FOLH1, Gi24, GITR, GITRL, GM-CSF, Her2, HHLA2, HMGB1, HVEM, ICOSLG, IDO, IFNα, IgE, IGF1R, IL2Rbeta, IL1, IL1A, IL1B, IL1F10, IL2, IL4, IL4Ra, IL5, IL5R, IL6, IL7, IL7Ra, IL8, IL9, IL9R, IL10, rhIL10, IL12, IL13, IL13Ra1, IL13Ra2, IL15, IL17, IL17Rb, IL18, IL22, IL23, IL25, IL27, IL33, IL35, ITGB4, ITK, KIR, LAG3, LAMP1, leptin, LPFS2, MHC class II, NCR3LG1, NKG2D, NTPDase-1, OX40, OX40L, PD-1H, platelet receptor, PROM1, S152, SISP1, SLC, SPG64, ST2, STEAP2, Syk kinase, TACI, TDO, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, WUCAM, and XCR1.

In another embodiment, the disclosure provides a method of purifying a binding protein produced by a host cell, comprising:
(a) producing in a host cell a binding protein comprising four polypeptide chains that form three antigen binding sites that specifically bind one or more antigen targets or target proteins, wherein a first polypeptide chain comprises a structure represented by the formula:

V_{L2}-L₁-V_{L1}-L₂-C_{L} [I]

and a second polypeptide chain comprises a structure represented by the formula:

V_{H1}-L₃-V_{H2}-L₄-C_{H1}-hinge-C_{H2}-C_{H3} [II]

and a third polypeptide chain comprises a structure represented by the formula:

V_{H3}-C_{H1} [III]

and a fourth polypeptide chain comprises a structure represented by the formula:

V_{L3}-C_{L} [IV]
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain;
C_{H2} is an immunoglobulin C_{H2} heavy chain constant domain;
C_{H3} is an immunoglobulin C_{H3} heavy chain constant domain;
hinge is an immunoglobulin hinge region connecting the C_{H1} and C_{H2} domains; and
L₁, L₂, L₃ and L₄ are amino acid linkers;
   wherein the polypeptide of formula I and the polypeptide of formula II form a cross-over light chain-heavy chain pair; wherein only one of the C_{H3} domain of the second polypeptide chain and the C_{H3} domain of the third polypeptide chain comprises amino acid substitutions at positions corresponding to positions 435 and 436 of human IgG1 or IgG4 according to EU Index, wherein the amino acid substitutions are H435R and Y436F;
(b) contacting the binding protein produced in (a) with Protein A; and
(c) eluting the binding protein from Protein A under conditions suitable for isolating the binding protein away from binding proteins comprising either 0 or 2 C_{H3} domains comprising the amino acid substitutions are H435R and Y436F.

In some embodiments, the C_{L} domain of the first polypeptide chain is a human kappa C_{L} domain, and the C_{L} domain of the fourth polypeptide chain is a human lambda C_{L} domain; or the C_{L} domain of the first polypeptide chain is a human lambda C_{L} domain, and the C_{L} domain of the fourth polypeptide chain is a human kappa C_{L} domain, and the method further comprises: (d) contacting the binding protein eluted in (c) with a kappa light chain affinity medium; and (e) eluting the binding protein from the kappa light chain affinity medium under conditions suitable for isolating the binding protein away from binding proteins comprising only lambda C_{L} domains. In some embodiments, the method further comprises, after (e), (f) contacting the binding protein eluted in (e) with a lambda light chain affinity medium; and (g) eluting the binding protein from the lambda light chain affinity medium under conditions suitable for isolating the binding protein away from binding proteins comprising only kappa C_{L} domains. In some embodiments, the C_{L} domain of the first polypeptide chain is a human kappa C_{L} domain, and the C_{L} domain of the fourth polypeptide chain is a human lambda C_{L} domain; or the C_{L} domain of the first polypeptide chain is a human lambda C_{L} domain, and the C_{L} domain of the fourth polypeptide chain is a human kappa C_{L} domain, and the method further comprises: (d) contacting the binding protein eluted in (c) with a lambda light chain affinity medium; and (e) eluting the binding protein from the lambda light chain affinity medium under conditions suitable for isolating the binding protein away from binding proteins comprising only kappa C_{L} domains. In some embodiments, the method further comprises, after (e), (f) contacting the binding protein eluted in (e) with a kappa light chain affinity medium; and (g) eluting the binding protein from the kappa light chain affinity medium under conditions suitable for isolating the binding protein away from binding proteins comprising only lambda C_{L} domains. In some embodiments, the first polypeptide chain comprises a lambda C_{L} domain; wherein the C_{H3} domain of the second polypeptide chain comprises amino acid substitutions at positions corresponding to positions 354 and 366 of human IgG1 according to EU Index, wherein the amino acid substitutions are S354C and T366W; wherein the C_{H3} domain of the third polypeptide chain comprises amino acid substitutions at positions corresponding to positions 349, 366, 368, 407, 435, and 436 of human IgG1 according to EU Index, wherein the amino acid substitutions are Y349C, T366S, L368A, Y407V, H435R, and Y436F; and wherein the fourth polypeptide chain comprises a kappa C_{L} domain. In some embodiments, the binding protein is detected in one or more of (c) and (e) using hydrophobic interaction chromatography (HIC). In some embodiments, the C_{H3} domains and/or Fc regions of the second and the third polypeptide chains are human IgG1 or IgG4 C_{H3} domains and/or Fc regions.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIGS. 1A-1C show schematic representations of trispecific binding proteins comprising four polypeptide chains that form three antigen binding sites that specifically binds three target proteins, wherein a first pair of polypeptides possess dual variable domains having a cross-over orientation (V_{H1}-V_{H2} and V_{L2}-V_{L1}) forming two antigen binding sites, and wherein a second pair of polypeptides possess a single variable domain (V_{H3} and V_{L3}) forming a single antigen binding site. FIG. 1A shows a trispecific binding protein comprising a “knobs-into-holes” mutation, where the knob is on the second pair of polypeptides with a single variable domain. FIG. 1B shows a trispecific binding protein comprising a “knobs-into-holes” mutation, where the knob is on the first pair of polypeptides having the cross-over orientation. FIG. 1C shows the orientation of variable domains on the polypeptide chains, and the knob/hole orientation for the binding proteins shown in Tables 1-3. “Heavy chain A” (e.g., a third polypeptide chain of the present disclosure) indicates the variable domain of heavy chain A. “Light chain A” (e.g., a fourth polypeptide chain of the present disclosure) indicates the variable domain of light chain A. “Heavy chain B” (e.g., a second polypeptide chain of the present disclosure) indicates variable domain 1 and variable domain 2 of heavy chain B. “Light chain B” (e.g., a first polypeptide chain of the present disclosure) indicates variable domain 1 and variable domain 2 of light chain B.

FIG. 2 shows the results of an ELISA assay determining the binding of an anti-Her2×CD28×CD3 IgG4 trispecific antibody (Binding Protein 1), or isotype control antibody, to human CD3, CD28 and Her2. The bound antibodies were detected using a horseradish peroxidase (HRP)-conjugated anti-Fab secondary antibody.

FIGS. 3A-3C show the results of antibody-mediated specific killing of Her2⁺ breast cancer cells using an anti-Her2×CD28×CD3 IgG4 trispecific antibody (referred to herein as “Binding protein 1”), an anti-CD28×CD3 IgG4 bispecific antibody (huCD28×CD3), an anti-Her2 IgG1 antibody, or a control antibody (human-IgG1), using human PBMC at the E:T=10. FIG. 3A shows the results of trispecific antibody-mediated specific killing of ZR-75-1 cells. FIG. 3B shows the results of trispecific antibody-mediated specific killing of AU565 cells. FIG. 3C shows the results of FACS analysis determining the cell surface expression of the indicated markers on ZR-75-1 and AU565 cells.

FIGS. 4 & 5 show the results of antibody-mediated specific killing of Her2⁺ breast cancer cells using an anti-Her2×CD28×CD3 IgG4 trispecific antibody (Binding protein 1), an anti-CD28×CD3 IgG4 bispecific antibody (huCD28×CD3), an anti-Her2 IgG1 antibody, or a control antibody (human-IgG1).

FIG. 4 shows the results of antibody-mediated specific killing of ZR-75-1 cells using human peripheral blood mononuclear cells (PBMCs) from donor KP45926 at E:T=10.

FIG. 5 shows the results of antibody-mediated specific killing of AU565 cells using human PBMCs from donor KP45944 at E:T=10.

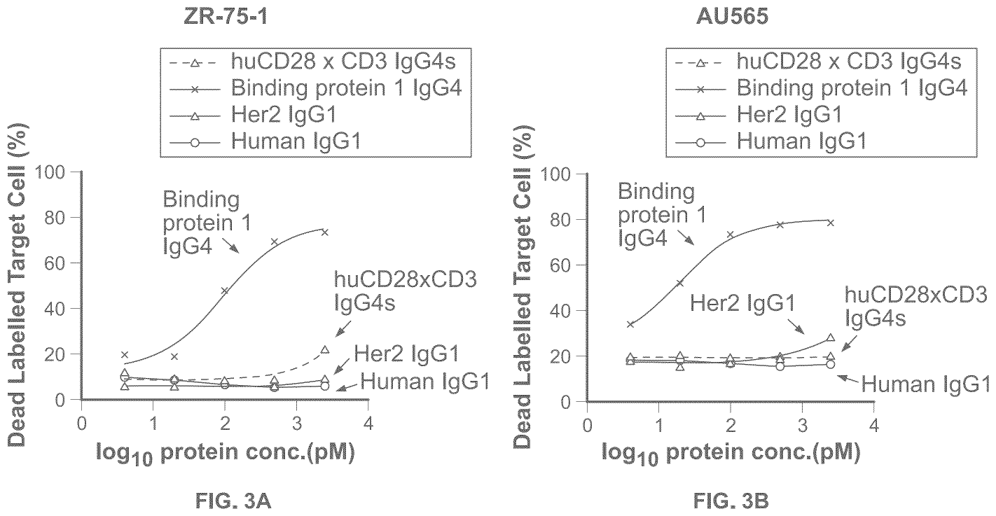

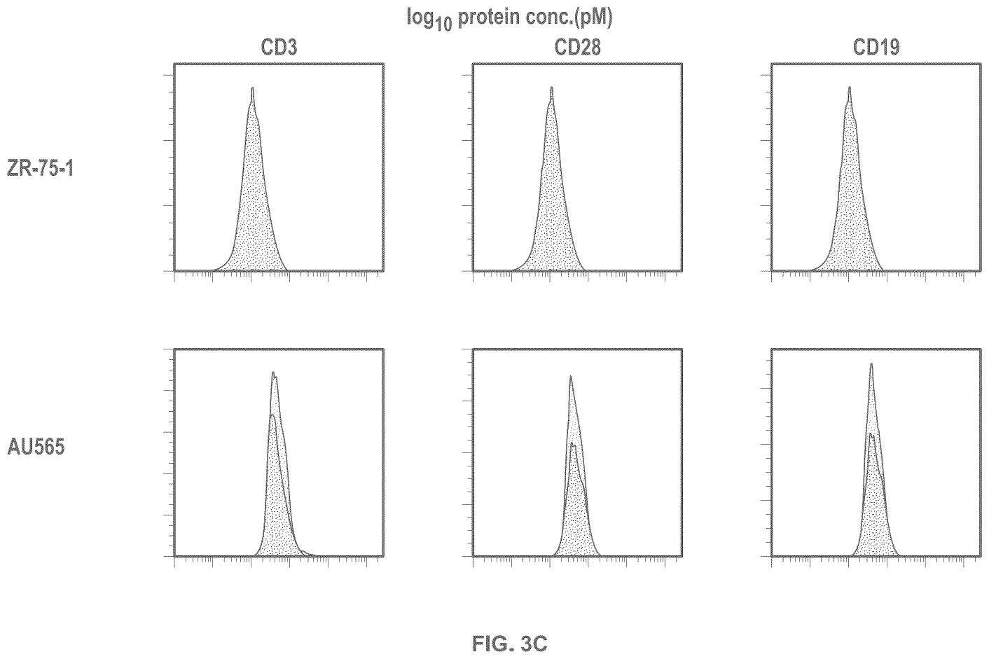

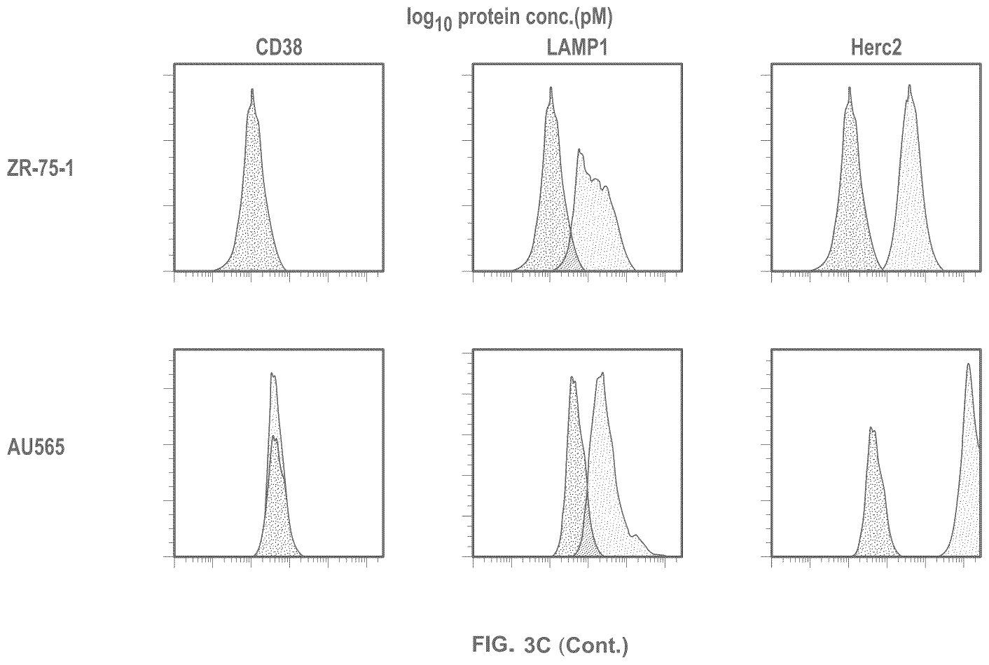

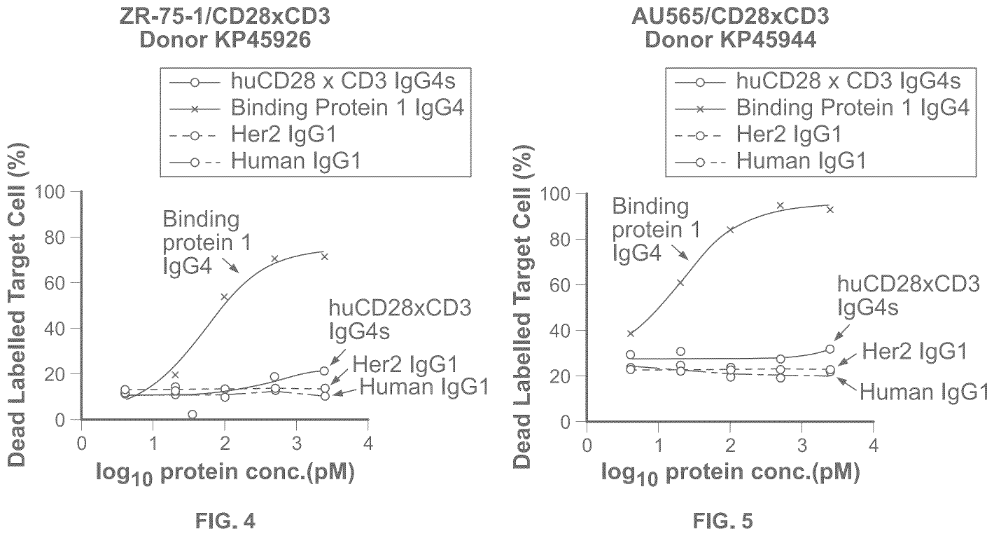

FIGS. 4 & 5 confirm similar cell killing results as shown in FIGS. 3A-3C using PBMCs from a different donor.

FIGS. 6A & 6B show the activation (CD69+) and proliferation of human T cells treated with anti-Her2×CD28×CD3 IgG4 trispecific binding protein (HER2/CD28ₛᵤₚ×CD3_{mid}; referred to herein as “Binding Protein 1”), anti-Her2×CD28×CD3 IgG4 trispecific binding protein lacking the anti-CD28 binding domain (HER2/ΔCD28ₛᵤₚ×CD3_{mid}), anti-Her2×CD28×CD3 IgG4 trispecific binding protein lacking the anti-CD3 binding domain (HER2/CD28ₛᵤₚ×ΔCD3_{mid}), anti-Her2×CD28×CD3 IgG4 trispecific binding protein lacking both anti-CD28 and anti-CD3 binding domains (HER2/Δ(CD28ₛᵤₚ×ΔCD3_{mid})), control anti-CD3 monoclonal antibody, or control IgG4 antibody. FIG. 6A shows the activation (CD69⁺) of human CD4⁺ T cells from three donors. FIG. 6B shows the activation (CD69⁺) of human CD8⁺ T cells from three donors. CD28ₛᵤₚ: anti-CD28 superagonist antibody. CD3_{mid}: anti-CD3 antibody.

FIGS. 7A-C show IL-2, NFκB, and nuclear factor of activated T-cells (NFAT) pathway activation via anti-CD3 and anti-CD28 signaling, as measured by luciferase assay using human Jurkat T cells with an IL-2 promoter-luciferase construct (FIG. 7A), an NFκB promoter-luciferase construct (FIG. 7B), or an NFAT promoter-luciferase construct (FIG. 7C). Antibodies tested were those described above in reference to FIGS. 6A-6D.

FIG. 8 shows the results of an ELISA assay determining binding of an anti-CD19×CD28×CD3 IgG4 trispecific antibody (referred to herein as “Binding Protein 3”), or isotype control antibody, to CD3, CD28, and CD19. The bound antibodies were detected using a horseradish peroxidase (HRP)-conjugated anti-Fab secondary antibody.

FIGS. 9A-9N show the results of antibody-mediated specific killing of CD19⁺ human GCB lymphoma cells using an anti-CD19×CD28×CD3 IgG4 trispecific antibody (referred to herein as “Binding Protein 3”), or the indicated controls, using human PBMC as effector cells at E:T=10. FIG. 9A shows the results of antibody-mediated specific killing of OCI-LY19 cells. FIG. 9B shows the results of FACS analysis determining the cell surface expression of the indicated markers on OCI-LY19 cells. FIG. 9C shows the results of antibody-mediated specific killing of OCI-LY19 cells using PBMCs from donor KP48572 at E:T=10. FIG. 9D shows the results of antibody-mediated specific killing of OCI-LY19 cells using PBMCs from donor KP48573 at E:T=10. FIG. 9E shows the results of antibody-mediated specific killing of human lymphoma KARPASS-422 cells using PBMCs from donor KP48572 at E:T=10. FIG. 9F shows the results of antibody-mediated specific killing of KARPASS-422 cells using PBMCs from donor KP48573 at E:T=10. FIG. 9G shows the results of antibody-mediated specific killing of human chronic B cell leukemia MeC1 cells using PBMCs from donor KP48572 at E:T=10. FIG. 9H shows the results of antibody-mediated specific killing of human multiple myeloma RPMI8226 cells using PBMCs from donor KP48775 at E:T=10. FIG. 9I shows the results of antibody-mediated specific killing of human Burkitt's lymphoma Raji cells using PBMCs from donor KP48572 at E:T=10. FIG. 9J shows the results of antibody-mediated specific killing of Human diffuse large B-cell lymphoma HBL1 cells using PBMCs from donor KP48775 at E:T=10. FIG. 9K shows the results of antibody-mediated specific killing of Large cell lymphoma SUDHL8 cells using PBMCs from donor KP48572 at E:T=10. FIG. 9L shows the results of antibody-mediated specific killing of SUDHL8 cells using PBMCs from donor KP48573 at E:T=10. FIG. 9M shows the results of antibody-mediated specific killing of human B cell lymphoma ARH77 cells using PBMCs from donor KP48775 at E:T=10. FIG. 9N shows the results of antibody-mediated specific killing of OCI-Ly3 cells using PBMCs from donor KP48775 at E:T=10.

FIG. 10 shows the results of an ELISA assay determining binding of an anti-CD38×CD28×CD3 IgG4 trispecific antibody (Binding Protein 5), or isotype control antibody, to CD3, CD28 and CD38. The bound antibodies were detected using a horseradish peroxidase (HRP)-conjugated anti-Fab secondary antibody.

FIGS. 11A-11D show the results of antibody-mediated specific killing of CD38⁺ human multiple myeloma cancer cells using an anti-CD38×CD28×CD3 IgG4 trispecific antibody (Binding protein 5), an anti-CD28×CD3 IgG4 bispecific antibody (huCD28×CD3), an anti-CD38 IgG1 antibody, or a control antibody (human-IgG1). FIG. 11A shows the results of antibody-mediated specific killing of MOLP-8 cells using human PBMC as effector cells at E:T=10. FIG. 11B shows the results of antibody-mediated specific killing of RPMI-8226 cells using human PBMC as effector cells at E:T=10. FIG. 11C shows the results of antibody-mediated specific killing of KMS-12-BM cells using human PBMC as effector cells at E:T=10. FIG. 11D shows the results of FACS analysis determining the cell surface expression of the indicated markers on MOLP-8, RPMI-8226, and KMS-12-BM cells.

FIGS. 12A-12D show the results of antibody-mediated specific killing of CD38⁺ human multiple myeloma cancer cells using an anti-CD38×CD28×CD3 IgG4 trispecific antibody (Binding protein 5), an anti-CD28×CD3 IgG4 bispecific antibody (huCD28×CD3), an anti-CD38 IgG1 antibody, or a control antibody (human-IgG1), using human PBMC as effector cells at E:T=10. FIG. 12A shows the results of antibody-mediated specific killing of NCI-H929 cells. FIG. 12B shows the results of antibody-mediated specific killing of MM.1S cells. FIG. 12C shows the results of antibody-mediated specific killing of MM.1R cells. FIG. 12D shows the results of FACS analysis determining the cell surface expression of the indicated markers on NCI-H929, MM.1S, and MM.1R cells.

FIGS. 13A-13D show the results of antibody-mediated specific killing of CD38⁺ human multiple myeloma cancer cells using an anti-CD38×CD28×CD3 IgG4 trispecific antibody (Binding protein 5), an anti-CD28×CD3 IgG4 bispecific antibody (huCD28×CD3), an anti-CD38 IgG1 antibody, or a control antibody (human-IgG1), using human PBMC as effector cells at E:T=10. FIG. 13A shows the results of antibody-mediated specific killing of OPM-2 cells. FIG. 13B shows the results of antibody-mediated specific killing of KMS-26 cells. FIG. 13C shows the results of antibody-mediated specific killing of U266 cells. FIG. 13D shows the results of FACS analysis determining the cell surface expression of the indicated markers on OPM-2, KMS-26, and U226 cells.

FIGS. 14A-14C show the results of antibody-mediated specific killing of CD38⁺ human lymphoma cancer cells using an anti-CD38×CD28×CD3 IgG4 trispecific antibody (Binding protein 5), an anti-CD28×CD3 IgG4 bispecific antibody (huCD28×CD3), an anti-CD38 IgG1 antibody, or a control antibody (human-IgG1), using human PBMC as effector cells at E:T=10. FIG. 14A shows the results of antibody-mediated specific killing of SUDHL-8 cells. FIG. 14B shows the results of antibody-mediated specific killing of OCI-LY19 cells. FIG. 14C shows the results of FACS analysis determining the cell surface expression of the indicated markers on SUDHL-8 and OCI-LY19 cells.

FIGS. 15A-15D show the results of antibody-mediated specific killing of CD38⁺ ALL cancer cells using an anti-CD38×CD28×CD3 IgG4 trispecific antibody (Binding protein 5), an anti-CD28×CD3 IgG4 bispecific antibody (huCD28×CD3), an anti-CD38 IgG1 antibody, or a control antibody (human-IgG1), using human PBMC as effector cells at E:T=10. FIG. 15A shows the results of antibody-mediated specific killing of KOPN-8 cells. FIG. 15B shows the results of antibody-mediated specific killing of HAL-1 cells. FIG. 15C shows the results of antibody-mediated specific killing of CCRF-SB cells. FIG. 15D shows the results of FACS analysis determining the cell surface expression of the indicated markers on KOPN-8, HAL-1, and CCRF-SB cells.

FIG. 16 shows the results of antibody-mediated specific killing of CD38⁺ myeloma cancer cells using anti-CD38×CD28×CD3 IgG4 trispecific antibodies (referred to herein as “Binding protein 5” and “Binding Protein 6,” depending on the specific anti-CD28 binding domain used), an anti-CD28×CD3 IgG4 bispecific antibody (huCD28×CD3), an anti-CD38 IgG1 antibody, a control anti-CD38 IgG1 antibody, or a control antibody (human-IgG1), using PBMCs from donor PK45926 at E:T=10.

FIGS. 17A & 17B show IL-2, NFκB, and nuclear factor of activated T-cells (NFAT) pathway activation via anti-CD3 and anti-CD28 signaling, as measured by luciferase assay using human Jurkat T cells with an IL-2 promoter-luciferase construct (FIG. 17A) or an NFAT promoter-luciferase construct (FIG. 17B). Antibodies tested were anti-CD38×CD28×CD3 IgG4 trispecific IgG4 antibody (Binding Protein 5, labeled as “Tri-Ab”), anti-CD38×CD28×CD3 IgG4 trispecific IgG4 antibody lacking the CD28 binding domain (labeled as “Tri-Ab (ΔCD28)), anti-CD38×CD28×CD3 IgG4 trispecific IgG4 antibody lacking the CD3 binding domain (labeled as “Tri-Ab (ΔCD3)), and anti-CD38×CD28×CD3 IgG4 trispecific IgG4 antibody lacking the CD3 and CD28 binding domains (labeled as “Tri-Ab (ΔCD28×ΔCD3)). Luciferase assays were performed in duplicate for each of the indicated Tri-Abs.

FIGS. 18A-18E show the results of a dose escalation toxicity study using the anti-Her2×CD28×CD3 IgG4 trispecific antibody (referred to herein as “Binding protein 1”) in non-human primates (dose escalating from 0.1, 0.5, 2.5, 5, 10, to 100 μg/kg; animals labeled as “409” and “410”). FIG. 18A shows the results of circulating CD4⁺ T cells percentage in each animal, 6 hours post administering the anti-Her2×CD28×CD3 trispecific antibody. FIG. 18B shows the results of circulating CD8⁺ T cells percentage in each animal, 6 hours post administering the anti-Her2×CD28×CD3 trispecific antibody. FIG. 18C shows the results of the activation (CD69⁺) of circulating CD4⁺ T cells 6 hours post dosing. FIG. 18D shows the results of the activation (CD69⁺) of circulating CD8⁺ T cells 6 hours post dosing. FIG. 18E shows the inflammatory cytokine release observed 6 hours post administering the anti-Her2×CD28×CD3 trispecific antibody at each dosing.

FIGS. 19A-B show the in vivo anti-tumor activity of the anti-Her2×CD28×CD3 IgG4 trispecific antibody (referred to herein as “Binding protein 1”) in the CD34⁺ umbilical cord blood cell humanized NSG mouse model implanted with BT474 cells. FIG. 19A shows the change in body weight of mice treated with the indicated concentrations of the anti-Her2×CD28×CD3 trispecific binding protein or PBS control. FIG. 19B shows the change in tumor volume in mice treated with the indicated concentrations of the anti-Her2×CD28×CD3 trispecific binding protein or PBS control.

FIGS. 20A-20H show the in vivo anti-tumor activity of the anti-Her2×CD28×CD3 IgG4 trispecific antibody (referred to herein as “Binding protein 1”) in the human PBMCs humanized NSG mouse model implanted with BT474 cells. FIG. 20A shows the effect of administering the indicated concentrations of the anti-Her2×CD28×CD3 trispecific binding protein, the indicated concentrations of Herceptin, or vehicle control, on the body weight of the mice. FIG. 20B shows the dose-dependent anti-tumor activity of the anti-Her2×CD28×CD3 trispecific binding protein, Herceptin or indicated controls, as in individual mice. FIG. 20C shows the average tumor volume in the mice after administration of the indicated concentrations of the anti-Her2×CD28×CD3 trispecific binding protein or PBS control. FIG. 20D shows the average tumor volume in the mice after administration of the indicated concentrations of Herceptin or PBS control. FIG. 20E shows bar graphs of the average tumor volume at day 34 in the mice after administration of the indicated concentrations of the anti-Her2×CD28×CD3 trispecific binding protein, the indicated concentrations of Herceptin, or PBS control. FIG. 20F shows the average tumor weight at day 34 in the mice after administration of the indicated concentrations of the anti-Her2×CD28×CD3 trispecific binding protein, the indicated concentrations of Herceptin, or PBS control. FIG. 20G shows the human CD45+, CD3+, CD4+, CD8+ cells in the blood of the mice at the end of the study. FIG. 20H shows the human CD45+, CD3+, CD4+, CD8+ cells in the spleens of the mice at the end of the study.

FIGS. 21A-F show the results of a dose escalation toxicity study using the anti-CD38×CD28×CD3 IgG4 trispecific antibody (Binding protein 5) in non-human primates (dose escalating from 0.1, 0.5, 2.5, 5, 10, to 100 μg/kg). FIG. 21A shows T cell activation (CD69⁺) (line graph) and proliferation (bar graph) of circulating CD4⁺ T cells after administration of the anti-CD38×CD28×CD3 trispecific antibody. FIG. 21B shows T cell activation (CD69⁺) (line graph) and proliferation (bar graph) of circulating CD8⁺ T cells after administration of the anti-CD38×CD28×CD3 trispecific antibody. FIG. 21C shows IL6 release in animals receiving the anti-CD38×CD28×CD3 trispecific antibody 6 hours post each dosing by individual animal. FIG. 21D shows IL10 release in animals receiving the anti-CD38×CD28×CD3 trispecific antibody 6 hours post each dosing by individual animal. FIG. 21E shows TNFa release in animals receiving the anti-CD38×CD28×CD3 trispecific antibody. FIG. 21F shows IFNγ release in animals receiving the anti-CD38×CD28×CD3 trispecific antibody 6 hours post each dosing by individual animal.

FIGS. 22A-22C show the in vivo anti-tumor activity of the anti-CD38×CD28×CD3 IgG4 trispecific antibody (Binding protein 5) in the CD34+ umbilical cord blood cells humanized NSG mouse model implanted with RPMI-8226 multiple myeloma cells transduced with CD38 and PD-L₁. As a pilot study, this experiment determined the working dose range for the Binding protein 5. FIG. 22A shows the in vivo tumor growth curve in groups of the indicated concentrations of the anti-CD38×CD28×CD3 trispecific binding protein or controls. FIG. 22B shows tumor infiltrating human CD8⁺ T cells in mice administered the anti-CD38×CD28×CD3 trispecific binding protein or the indicated controls. FIG. 22C shows tumor infiltrating human CD4⁺ T cells in mice administered the anti-CD38×CD28×CD3 trispecific binding protein or the indicated controls.

FIGS. 23A-23D show the in vivo activity of the anti-CD38×CD28×CD3 IgG4 trispecific antibody (Binding protein 5) in the CD34⁺ umbilical cord blood cells humanized NSG mouse model implanted with RPMI-8226 cells transduced with CD38 and PD-L1. FIG. 23A shows the change in body weight of mice treated with the indicated concentrations of the anti-CD38×CD28×CD3 trispecific binding protein or PBS control. FIG. 23B shows the change in tumor volume in mice treated with the indicated concentrations of the anti-CD38×CD28×CD3 trispecific binding protein or PBS control. FIG. 23C shows the tumor volumes in each group at Day 19. The tumor volumes in all treated groups showed marked reduction, which are statistically different form the PBS control group. FIG. 23D shows the serum concentration of inflammatory cytokines IFN-g, TNF, and IL-2 in mice four hours after the first dose of the indicated concentrations of the anti-CD38×CD28×CD3 trispecific binding protein or PBS control.

FIGS. 24 & 25 show the in vivo activation of T cells in the CD34+ umbilical cord blood cells humanized NSG mouse model by administering an anti-CD38×CD28×CD3 IgG4 trispecific antibody (Binding protein 5; triangles), an anti-CD28×CD3 IgG4 bispecific antibody (squares), or an anti-CD28 IgG4 antibody (circles) by determining the increase in the percentage of CD69⁺ T cells.

FIG. 24 shows the in vivo activation of CD4⁺ T cells.

FIG. 25 shows the in vivo activation of CD8⁺ T cells.

FIGS. 26A-26C show the in vivo activation of T cells in the CD34+ umbilical cord blood cells humanized NSG mouse model by administering an anti-CD38×CD28×CD3 IgG4 trispecific antibody (Binding protein 5; triangles), an anti-CD28×CD3 IgG4 bispecific antibody (squares), or an anti-CD28 IgG4 antibody (circles) by determining the serum levels of inflammatory cytokines. FIG. 26A shows the serum levels of IL-2. FIG. 26B shows the serum levels of TNF. FIG. 26C shows the serum levels of IFN-γ.

FIGS. 27A & 27B show the purification of the indicated proteins by size exclusion chromatography. FIG. 27A shows the purification of Binding Proteins 9-15 by size exclusion chromatography. FIG. 27B shows the purification of Binding Proteins 16-19 by size exclusion chromatography.

FIG. 28A depicts the trispecific binding protein used in experiments for optimizing a purification scheme and configuration of optional binding protein features (e.g., kappa/lambda light chains, knob/hole mutations, and H435R/Y436F mutations).

FIG. 28B shows each of the configurations tested.

FIG. 29 shows representative chromatograms from analytical hydrophobic interaction chromatography (HIC), demonstrating that trispecific binding proteins were distinguishable from mispaired species.

FIGS. 30A & 30B show the successful purification of a binding protein with lambda light chain for CODV arm, kappa light chain for Fab arm, knob mutations on CODV arm, hole mutations on Fab arm, and RF mutations on Fab arm by Protein A followed by KappaSelect (GE Healthcare) purification steps. Successful purification of binding protein from mispaired species was demonstrated by hydrophobic interaction chromatography (HIC; FIG. 30A) and SDS-PAGE (FIG. 30B).

### DETAILED DESCRIPTION

The disclosure provides trispecific and/or trivalent binding proteins comprising four polypeptide chains that form three antigen binding sites that specifically bind to one or more target proteins, wherein a first pair of polypeptides forming the binding protein possess dual variable domains having a cross-over orientation and wherein a second pair of polypeptides forming the binding protein possess a single variable domain.

### General Definitions

As utilized in accordance with the present disclosure, the following terms, unless otherwise indicated, shall be understood to have the following meanings. Unless otherwise required by context, singular terms shall include pluralities and plural terms shall include the singular.

The term “polynucleotide” as used herein refers to single-stranded or double-stranded nucleic acid polymers of at least 10 nucleotides in length. In certain embodiments, the nucleotides comprising the polynucleotide can be ribonucleotides or deoxyribonucleotides or a modified form of either type of nucleotide. Such modifications include base modifications such as bromuridine, ribose modifications such as arabinoside and 2′,3′-dideoxyribose, and internucleotide linkage modifications such as phosphorothioate, phosphorodithioate, phosphoroselenoate, phosphorodiselenoate, phosphoroanilothioate, phoshoraniladate and phosphoroamidate. The term “polynucleotide” specifically includes single-stranded and double-stranded forms of DNA.

An “isolated polynucleotide” is a polynucleotide of genomic, cDNA, or synthetic origin or some combination thereof, which: (1) is not associated with all or a portion of a polynucleotide in which the isolated polynucleotide is found in nature, (2) is linked to a polynucleotide to which it is not linked in nature, or (3) does not occur in nature as part of a larger sequence.

An “isolated polypeptide” is one that: (1) is free of at least some other polypeptides with which it would normally be found, (2) is essentially free of other polypeptides from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or noncovalent interaction) with portions of a polypeptide with which the “isolated polypeptide” is associated in nature, (6) is operably associated (by covalent or noncovalent interaction) with a polypeptide with which it is not associated in nature, or (7) does not occur in nature. Such an isolated polypeptide can be encoded by genomic DNA, cDNA, mRNA or other RNA, of synthetic origin, or any combination thereof. Preferably, the isolated polypeptide is substantially free from polypeptides or other contaminants that are found in its natural environment that would interfere with its use (therapeutic, diagnostic, prophylactic, research or otherwise).

Naturally occurring antibodies typically comprise a tetramer. Each such tetramer is typically composed of two identical pairs of polypeptide chains, each pair having one full-length “light” chain (typically having a molecular weight of about 25 kDa) and one full-length “heavy” chain (typically having a molecular weight of about 50-70 kDa). The terms “heavy chain” and “light chain” as used herein refer to any immunoglobulin polypeptide having sufficient variable domain sequence to confer specificity for a target antigen. The amino-terminal portion of each light and heavy chain typically includes a variable domain of about 100 to 110 or more amino acids that typically is responsible for antigen recognition. The carboxy-terminal portion of each chain typically defines a constant domain responsible for effector function. Thus, in a naturally occurring antibody, a full-length heavy chain immunoglobulin polypeptide includes a variable domain (VH) and three constant domains (C_{H1}, C_{H2}, and C_{H3}), wherein the VH domain is at the amino-terminus of the polypeptide and the C_{H3} domain is at the carboxyl-terminus, and a full-length light chain immunoglobulin polypeptide includes a variable domain (V_{L}) and a constant domain (C_{L}), wherein the V_{L} domain is at the amino-terminus of the polypeptide and the C_{L} domain is at the carboxyl-terminus.

Human light chains are typically classified as kappa and lambda light chains, and human heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2. IgA is similarly subdivided into subclasses including, but not limited to, IgA1 and IgA2. Within full-length light and heavy chains, the variable and constant domains typically are joined by a “J” region of about 12 or more amino acids, with the heavy chain also including a “D” region of about 10 more amino acids. See, e.g., FUNDAMENTAL IMMUNOLOGY (Paul, W., ed., Raven Press, 2nd ed., 1989), which is incorporated by reference in its entirety for all purposes. The variable regions of each light/heavy chain pair typically form an antigen binding site. The variable domains of naturally occurring antibodies typically exhibit the same general structure of relatively conserved framework regions (FR) joined by three hypervariable regions, also called complementarity determining regions or CDRs. The CDRs from the two chains of each pair typically are aligned by the framework regions, which may enable binding to a specific epitope. From the amino-terminus to the carboxyl-terminus, both light and heavy chain variable domains typically comprise the domains FR1, CDR1, FR2, CDR2, FR3, CDR3, and FR4.

The term “CDR set” refers to a group of three CDRs that occur in a single variable region capable of binding the antigen. The exact boundaries of these CDRs have been defined differently according to different systems. The system described by Kabat (Kabat et al., SEQUENCES OF PROTEINS OF IMMUNOLOGICAL INTEREST (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia and Lesk, 1987, J. Mol. Biol. 196: 901-17; Chothia et al., 1989, Nature 342: 877-83) found that certain sub-portions within Kabat CDRs adopt nearly identical peptide backbone conformations, despite having great diversity at the level of amino acid sequence. These sub-portions were designated as L1, L2, and L3 or H1, H2, and H3 where the “L” and the “H” designates the light chain and the heavy chain regions, respectively. These regions may be referred to as Chothia CDRs, which have boundaries that overlap with Kabat CDRs. Other boundaries defining CDRs overlapping with the Kabat CDRs have been described by Padlan, 1995, FASEB J. 9: 133-39; MacCallum, 1996, J. Mol. Biol. 262(5): 732-45; and Lefranc, 2003, Dev. Comp. Immunol. 27: 55-77. Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat or Chothia defined CDRs. Identification of predicted CDRs using the amino acid sequence is well known in the field, such as in Martin, A. C. “Protein sequence and structure analysis of antibody variable domains,” In Antibody Engineering, Vol. 2. Kontermann R., Diibel S., eds. Springer-Verlag, Berlin, p. 33-51 (2010). The amino acid sequence of the heavy and/or light chain variable domain may be also inspected to identify the sequences of the CDRs by other conventional methods, e.g., by comparison to known amino acid sequences of other heavy and light chain variable regions to determine the regions of sequence hypervariability. The numbered sequences may be aligned by eye, or by employing an alignment program such as one of the CLUSTAL suite of programs, as described in Thompson, 1994, Nucleic Acids Res. 22: 4673-80. Molecular models are conventionally used to correctly delineate framework and CDR regions and thus correct the sequence-based assignments.

The term “Fc” as used herein refers to a molecule comprising the sequence of a non-antigen-binding fragment resulting from digestion of an antibody or produced by other means, whether in monomeric or multimeric form, and can contain the hinge region. The original immunoglobulin source of the native Fc is preferably of human origin and can be any of the immunoglobulins, although IgG1 and IgG2 are preferred. Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, and IgE) or subclass (e.g., IgG1, IgG2, IgG3, IgA1, and IgGA2). One example of a Fc is a disulfide-bonded dimer resulting from papain digestion of an IgG. The term “native Fc” as used herein is generic to the monomeric, dimeric, and multimeric forms.

A F(ab) fragment typically includes one light chain and the V_{H} and C_{H1} domains of one heavy chain, wherein the V_{H}-C_{H1} heavy chain portion of the F(ab) fragment cannot form a disulfide bond with another heavy chain polypeptide. As used herein, a F(ab) fragment can also include one light chain containing two variable domains separated by an amino acid linker and one heavy chain containing two variable domains separated by an amino acid linker and a C_{H1} domain.

A F(ab′) fragment typically includes one light chain and a portion of one heavy chain that contains more of the constant region (between the C_{H1} and C_{H2} domains), such that an interchain disulfide bond can be formed between two heavy chains to form a F(ab′)₂ molecule.

The term “binding protein” as used herein refers to a non-naturally occurring (or recombinant or engineered) molecule that specifically binds to at least one target antigen, and which comprises four polypeptide chains that form at least three antigen binding sites, wherein a first polypeptide chain has a structure represented by the formula:

V_{L2}-L₁-V_{L1}-L₂-C_{L} [I]

and a second polypeptide chain has a structure represented by the formula:

V_{H1}-L₃-V_{H2}-L₄-C_{H1} [II]

and a third polypeptide chain has a structure represented by the formula:

V_{H3}-C_{H1} [III]

and a fourth polypeptide chain has a structure represented by the formula:

V_{L3}-C_{L} [IV]

wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain; and
L₁, L₂, L₃ and L₄ are amino acid linkers;
and wherein the polypeptide of formula I and the polypeptide of formula II form a cross-over light chain-heavy chain pair.

A “recombinant” molecule is one that has been prepared, expressed, created, or isolated by recombinant means.

One embodiment of the disclosure provides binding proteins having biological and immunological specificity to between one and three target antigens. Another embodiment of the disclosure provides nucleic acid molecules comprising nucleotide sequences encoding polypeptide chains that form such binding proteins. Another embodiment of the disclosure provides expression vectors comprising nucleic acid molecules comprising nucleotide sequences encoding polypeptide chains that form such binding proteins. Yet another embodiment of the disclosure provides host cells that express such binding proteins (i.e., comprising nucleic acid molecules or vectors encoding polypeptide chains that form such binding proteins).

The term “swapability” as used herein refers to the interchangeability of variable domains within the binding protein format and with retention of folding and ultimate binding affinity. “Full swapability” refers to the ability to swap the order of both V_{H1} and V_{H2} domains, and therefore the order of V_{L1} and V_{L2} domains, in the polypeptide chain of formula I or the polypeptide chain of formula II (i.e., to reverse the order) while maintaining full functionality of the binding protein as evidenced by the retention of binding affinity. Furthermore, it should be noted that the designations V_{H} and V_{L} refer only to the domain's location on a particular protein chain in the final format. For example, V_{H1} and V_{H2} could be derived from V_{L1} and V_{L2} domains in parent antibodies and placed into the V_{H1} and V_{H2} positions in the binding protein. Likewise, V_{L1} and V_{L2} could be derived from V_{H1} and V_{H2} domains in parent antibodies and placed in the V_{H1} and V_{H2} positions in the binding protein. Thus, the V_{H} and V_{L} designations refer to the present location and not the original location in a parent antibody. V_{H} and V_{L} domains are therefore “swappable.”

The term “antigen” or “target antigen” or “antigen target” as used herein refers to a molecule or a portion of a molecule that is capable of being bound by a binding protein, and additionally is capable of being used in an animal to produce antibodies capable of binding to an epitope of that antigen. A target antigen may have one or more epitopes. With respect to each target antigen recognized by a binding protein, the binding protein is capable of competing with an intact antibody that recognizes the target antigen.

The term “Her2” refers to human epidermal growth factor receptor 2 which is a member of the epidermal growth factor receptor family.

“CD3” is cluster of differentiation factor 3 polypeptide and is a T-cell surface protein that is typically part of the T cell receptor (TCR) complex.

“CD28” is cluster of differentiation 28 polypeptide and is a T-cell surface protein that provides co-stimulatory signals for T-cell activation and survival.

“CD19” is cluster of differentiation 19 polypeptide and is located on B-cells.

“CD20” is cluster of differentiation 20 polypeptide and is an activated-glycosylated phosphoprotein expressed on the surface of B-cells.

“CD38” is cluster of differentiation 38 polypeptide and is a glycoprotein found on the surface of many immune cells.

“LAMP1” is lysosomal-associated membrane protein 1.

“IL-4” is interleukin 4 and is a cytokine that induces differentiation of naïve helper T cells.

“IL-13” is interleukin 13 and is a cytokine secreted by many cell types such as T-cells.

“TNFa” is tumor necrosis factor alpha and is a cytokine involved in systematic inflammation.

The term “T-cell engager” refers to binding proteins directed to a host's immune system, more specifically the T cells' cytotoxic activity as well as directed to a tumor target protein.

The term “monospecific binding protein” refers to a binding protein that specifically binds to one antigen target.

The term “monovalent binding protein” refers to a binding protein that has one antigen binding site.

The term “bispecific binding protein” refers to a binding protein that specifically binds to two different antigen targets.

The term “bivalent binding protein” refers to a binding protein that has two binding sites.

The term “trispecific binding protein” refers to a binding protein that specifically binds to three different antigen targets.

The term “trivalent binding protein” refers to a binding protein that has three binding sites. In particular embodiments the trivalent binding protein can bind to one antigen target. In other embodiments, the trivalent binding protein can bind to two antigen targets. In other embodiments, the trivalent binding protein can bind to three antigen targets.

An “isolated” binding protein is one that has been identified and separated and/or recovered from a component of its natural environment. Contaminant components of its natural environment are materials that would interfere with diagnostic or therapeutic uses for the binding protein, and may include enzymes, hormones, and other proteinaceous or non-proteinaceous solutes. In some embodiments, the binding protein will be purified: (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated binding proteins include the binding protein in situ within recombinant cells since at least one component of the binding protein's natural environment will not be present.

The terms “substantially pure” or “substantially purified” as used herein refer to a compound or species that is the predominant species present (i.e., on a molar basis it is more abundant than any other individual species in the composition). In some embodiments, a substantially purified fraction is a composition wherein the species comprises at least about 50% (on a molar basis) of all macromolecular species present. In other embodiments, a substantially pure composition will comprise more than about 80%, 85%, 90%, 95%, or 99% of all macromolar species present in the composition. In still other embodiments, the species is purified to essential homogeneity (contaminant species cannot be detected in the composition by conventional detection methods) wherein the composition consists essentially of a single macromolecular species.

A “neutralizing” binding protein as used herein refers to a molecule that is able to block or substantially reduce an effector function of a target antigen to which it binds. As used herein, “substantially reduce” means at least about 60%, preferably at least about 70%, more preferably at least about 75%, even more preferably at least about 80%, still more preferably at least about 85%, most preferably at least about 90% reduction of an effector function of the target antigen.

The term “epitope” includes any determinant, preferably a polypeptide determinant, capable of specifically binding to an immunoglobulin or T-cell receptor. In certain embodiments, epitope determinants include chemically active surface groupings of molecules such as amino acids, sugar side chains, phosphoryl groups, or sulfonyl groups, and, in certain embodiments, may have specific three-dimensional structural characteristics and/or specific charge characteristics. An epitope is a region of an antigen that is bound by an antibody or binding protein. In certain embodiments, a binding protein is said to specifically bind an antigen when it preferentially recognizes its target antigen in a complex mixture of proteins and/or macromolecules. In some embodiments, a binding protein is said to specifically bind an antigen when the equilibrium dissociation constant is ≤10⁻⁸ M, more preferably when the equilibrium dissociation constant is ≤10⁻⁹ M, and most preferably when the dissociation constant is ≤10⁻¹⁰ M.

The dissociation constant (K_{D}) of a binding protein can be determined, for example, by surface plasmon resonance. Generally, surface plasmon resonance analysis measures real-time binding interactions between ligand (a target antigen on a biosensor matrix) and analyte (a binding protein in solution) by surface plasmon resonance (SPR) using the BIAcore system (Pharmacia Biosensor; Piscataway, NJ). Surface plasmon analysis can also be performed by immobilizing the analyte (binding protein on a biosensor matrix) and presenting the ligand (target antigen). The term “K_{D},” as used herein refers to the dissociation constant of the interaction between a particular binding protein and a target antigen.

The term “specifically binds” as used herein refers to the ability of a binding protein or an antigen-binding fragment thereof to bind to an antigen containing an epitope with an Kd of at least about 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, or more, and/or to bind to an epitope with an affinity that is at least two-fold greater than its affinity for a nonspecific antigen.

The term “linker” as used herein refers to one or more amino acid residues inserted between immunoglobulin domains to provide sufficient mobility for the domains of the light and heavy chains to fold into cross over dual variable region immunoglobulins. A linker is inserted at the transition between variable domains or between variable and constant domains, respectively, at the sequence level. The transition between domains can be identified because the approximate size of the immunoglobulin domains are well understood. The precise location of a domain transition can be determined by locating peptide stretches that do not form secondary structural elements such as beta-sheets or alpha-helices as demonstrated by experimental data or as can be assumed by techniques of modeling or secondary structure prediction. The linkers described herein are referred to as L₁, which is located on the light chain between the C-terminus of the V_{L2} and the N-terminus of the V_{L1} domain; and L₂, which is located on the light chain between the C-terminus of the V_{L1} and the N-terminus of the C_{L} domain. The heavy chain linkers are known as L₃, which is located between the C-terminus of the V_{H1} and the N-terminus of the V_{H2} domain; and L₄, which is located between the C-terminus of the V_{H2} and the N-terminus of the C_{H1} domain.

The term “vector” as used herein refers to any molecule (e.g., nucleic acid, plasmid, or virus) that is used to transfer coding information to a host cell. The term “vector” includes a nucleic acid molecule that is capable of transporting another nucleic acid to which it has been linked. One type of vector is a “plasmid,” which refers to a circular double-stranded DNA molecule into which additional DNA segments may be inserted. Another type of vector is a viral vector, wherein additional DNA segments may be inserted into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell and thereby are replicated along with the host genome. In addition, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. The terms “plasmid” and “vector” may be used interchangeably herein, as a plasmid is the most commonly used form of vector. However, the disclosure is intended to include other forms of expression vectors, such as viral vectors (e.g., replication defective retroviruses, adenoviruses, and adeno-associated viruses), which serve equivalent functions.

The phrase “recombinant host cell” (or “host cell”) as used herein refers to a cell into which a recombinant expression vector has been introduced. A recombinant host cell or host cell is intended to refer not only to the particular subject cell, but also to the progeny of such a cell. Because certain modifications may occur in succeeding generations due to either mutation or environmental influences, such progeny may not, in fact, be identical to the parent cell, but such cells are still included within the scope of the term “host cell” as used herein. A wide variety of host cell expression systems can be used to express the binding proteins, including bacterial, yeast, baculoviral, and mammalian expression systems (as well as phage display expression systems). An example of a suitable bacterial expression vector is pUC19. To express a binding protein recombinantly, a host cell is transformed or transfected with one or more recombinant expression vectors carrying DNA fragments encoding the polypeptide chains of the binding protein such that the polypeptide chains are expressed in the host cell and, preferably, secreted into the medium in which the host cells are cultured, from which medium the binding protein can be recovered.

The term “transformation” as used herein refers to a change in a cell's genetic characteristics, and a cell has been transformed when it has been modified to contain a new DNA. For example, a cell is transformed where it is genetically modified from its native state. Following transformation, the transforming DNA may recombine with that of the cell by physically integrating into a chromosome of the cell, or may be maintained transiently as an episomal element without being replicated, or may replicate independently as a plasmid. A cell is considered to have been stably transformed when the DNA is replicated with the division of the cell. The term “transfection” as used herein refers to the uptake of foreign or exogenous DNA by a cell, and a cell has been “transfected” when the exogenous DNA has been introduced inside the cell membrane. A number of transfection techniques are well known in the art. Such techniques can be used to introduce one or more exogenous DNA molecules into suitable host cells.

The term “naturally occurring” as used herein and applied to an object refers to the fact that the object can be found in nature and has not been manipulated by man. For example, a polynucleotide or polypeptide that is present in an organism (including viruses) that can be isolated from a source in nature and that has not been intentionally modified by man is naturally-occurring. Similarly, “non-naturally occurring” as used herein refers to an object that is not found in nature or that has been structurally modified or synthesized by man.

As used herein, the twenty conventional amino acids and their abbreviations follow conventional usage. Stereoisomers (e.g., D-amino acids) of the twenty conventional amino acids; unnatural amino acids and analogs such as α-, α-disubstituted amino acids, N-alkyl amino acids, lactic acid, and other unconventional amino acids may also be suitable components for the polypeptide chains of the binding proteins. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxyl-terminal direction, in accordance with standard usage and convention.

Naturally occurring residues may be divided into classes based on common side chain properties:
(1) hydrophobic: Met, Ala, Val, Leu, Ile, Phe, Trp, Tyr, Pro;
(2) polar hydrophilic: Arg, Asn, Asp, Gln, Glu, His, Lys, Ser, Thr;
(3) aliphatic: Ala, Gly, Ile, Leu, Val, Pro;
(4) aliphatic hydrophobic: Ala, Ile, Leu, Val, Pro;
(5) neutral hydrophilic: Cys, Ser, Thr, Asn, Gln;
(6) acidic: Asp, Glu;
(7) basic: His, Lys, Arg;
(8) residues that influence chain orientation: Gly, Pro;
(9) aromatic: His, Trp, Tyr, Phe; and
(10) aromatic hydrophobic: Phe, Trp, Tyr.

Conservative amino acid substitutions may involve exchange of a member of one of these classes with another member of the same class. Non-conservative substitutions may involve the exchange of a member of one of these classes for a member from another class.

A skilled artisan will be able to determine suitable variants of the polypeptide chains of the binding proteins using well-known techniques. For example, one skilled in the art may identify suitable areas of a polypeptide chain that may be changed without destroying activity by targeting regions not believed to be important for activity. Alternatively, one skilled in the art can identify residues and portions of the molecules that are conserved among similar polypeptides. In addition, even areas that may be important for biological activity or for structure may be subject to conservative amino acid substitutions without destroying the biological activity or without adversely affecting the polypeptide structure.

The term “patient” as used herein includes human and animal subjects.

The terms “treatment” or “treat” as used herein refer to both therapeutic treatment and prophylactic or preventative measures. Those in need of treatment include those having a disorder as well as those prone to have the disorder or those in which the disorder is to be prevented. In particular embodiments, binding proteins can be used to treat humans with cancer, or humans susceptible to cancer, or ameliorate cancer in a human subject. The binding proteins can also be used to prevent cancer in a human patient. In particular embodiments, the cancer is multiple myeloma, acute lymphoblastic leukemia, chronic lymphocytic leukemia, acute myeloid leukemia, lymphoma, breast cancer such as Her2+ breast cancer, germinal center B-cell lymphoma or B-cell acute lymphoblastic leukemia, In other embodiments, the binding proteins can be used to treat humans with inflammatory disorders, or humans susceptible to inflammatory disorders, or ameliorate inflammatory disorders in a human subject.

The terms “pharmaceutical composition” or “therapeutic composition” as used herein refer to a compound or composition capable of inducing a desired therapeutic effect when properly administered to a patient.

The term “pharmaceutically acceptable carrier” or “physiologically acceptable carrier” as used herein refers to one or more formulation materials suitable for accomplishing or enhancing the delivery of a binding protein.

The terms “effective amount” and “therapeutically effective amount” when used in reference to a pharmaceutical composition comprising one or more binding proteins refer to an amount or dosage sufficient to produce a desired therapeutic result. More specifically, a therapeutically effective amount is an amount of a binding protein sufficient to inhibit, for some period of time, one or more of the clinically defined pathological processes associated with the condition being treated. The effective amount may vary depending on the specific binding protein that is being used, and also depends on a variety of factors and conditions related to the patient being treated and the severity of the disorder. For example, if the binding protein is to be administered in vivo, factors such as the age, weight, and health of the patient as well as dose response curves and toxicity data obtained in preclinical animal work would be among those factors considered. The determination of an effective amount or therapeutically effective amount of a given pharmaceutical composition is well within the ability of those skilled in the art.

One embodiment of the disclosure provides a pharmaceutical composition comprising a pharmaceutically acceptable carrier and a therapeutically effective amount of a binding protein.

Trispecific and/or Trivalent Binding Proteins

In one embodiment, the binding protein of the disclosure is a trispecific and/or trivalent binding protein comprising four polypeptide chains that form three antigen binding sites that specifically bind one or more (e.g., three) different antigen targets or target proteins, wherein a first polypeptide chain comprises a structure represented by the formula:

V_{L2}-L₁-V_{L1}-L₂-C_{L} [I]

and a second polypeptide chain comprises a structure represented by the formula:

V_{H1}-L₃-V_{H2}-L₄-C_{H1} [II]

and a third polypeptide chain comprises a structure represented by the formula:

V_{H3}-C_{H1} [III]

and a fourth polypeptide chain comprises a structure represented by the formula:

V_{L3}-C_{L} [IV]

wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain; and
L₁, L₂, L₃ and L₄ are amino acid linkers;
   and wherein the polypeptide of formula I and the polypeptide of formula II form a cross-over light chain-heavy chain pair.

In one embodiment, the binding protein of the disclosure is a trispecific and/or trivalent binding protein comprising four polypeptide chains that form three antigen binding sites that specifically bind one or more (e.g., three) antigen targets or target proteins, wherein a first polypeptide chain comprises a structure represented by the formula:

V_{L2}-L₁-V_{L1}-L₂-C_{L} [I]

and a second polypeptide chain comprises a structure represented by the formula:

V_{H1}-L₃-V_{H2}-L₄-C_{H1} [II]

and a third polypeptide chain comprises a structure represented by the formula:

V_{H3}-C_{H1} [III]

and a fourth polypeptide chain comprises a structure represented by the formula:

V_{L3}-C_{L} [IV]

wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain; and
L₁, L₂, L₃ and L₄ are amino acid linkers;
and wherein the polypeptide of formula I and the polypeptide of formula II form a cross-over light chain-heavy chain pair.

In one embodiment, the binding protein of the disclosure is a trispecific and/or trivalent binding protein comprising four polypeptide chains that form three antigen binding sites that specifically bind one or more (e.g., three) different antigen targets or target proteins, wherein a first polypeptide chain comprises a structure represented by the formula:

V_{L2}-L₁-V_{L1}-L₂-C_{L} [I]

and a second polypeptide chain comprises a structure represented by the formula:

V_{H1}-L₃-V_{H2}-L₄-C_{H1}-hinge-C_{H2}-C_{H3} [II]

and a third polypeptide chain comprises a structure represented by the formula:

V_{H3}-C_{H1}-hinge-C_{H2}-C_{H3} [III]

and a fourth polypeptide chain comprises a structure represented by the formula:

V_{L3}-C_{L} [IV]

wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain;
C_{H2} is an immunoglobulin C_{H2} heavy chain constant domain;
C_{H3} is an immunoglobulin C_{H3} heavy chain constant domain;
hinge is an immunoglobulin hinge region connecting the C_{H1} and C_{H2} domains; and
L₁, L₂, L₃ and L₄ are amino acid linkers;
and wherein the polypeptide of formula I and the polypeptide of formula II form a cross-over light chain-heavy chain pair.

In one embodiment, the binding protein of the disclosure is a trispecific and/or trivalent binding protein comprising four polypeptide chains that form three antigen binding sites that specifically bind one or more (e.g., three) antigen targets or target proteins, wherein a first polypeptide chain comprises a structure represented by the formula:

V_{L2}-L₁-V_{L1}-L₂-C_{L} [I]

and a second polypeptide chain comprises a structure represented by the formula:

V_{H1}-L₃-V_{H2}-L₄-C_{H1}-hinge-C_{H2}-C_{H3} [II]

and a third polypeptide chain comprises a structure represented by the formula:

V_{H3}-C_{H1}-hinge-C_{H2}-C_{H3} [III]

and a fourth polypeptide chain comprises a structure represented by the formula:

V_{L3}-C_{L} [IV]

wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain;
C_{H2} is an immunoglobulin C_{H2} heavy chain constant domain;
C_{H3} is an immunoglobulin C_{H3} heavy chain constant domain;
hinge is an immunoglobulin hinge region connecting the C_{H1} and C_{H2} domains; and
L₁, L₂, L₃ and L₄ are amino acid linkers;
   and wherein the polypeptide of formula I and the polypeptide of formula II form a cross-over light chain-heavy chain pair.

In some embodiments, the first polypeptide chain and the second polypeptide chain have a cross-over orientation that forms two distinct antigen binding sites. In some embodiments, the V_{H1} and V_{L1} form a binding pair and form the first antigen binding site. In some embodiments, the V_{H2} and V_{L2} form a binding pair and form the second antigen binding site. In some embodiments, the third polypeptide and the fourth polypeptide form a third antigen binding site. In some embodiments, the V_{H3} and V_{L3} form a binding pair and form the third antigen binding site.

In one embodiment, the binding protein of the disclosure is a trispecific and/or trivalent binding protein comprising four polypeptide chains that form three antigen binding sites that specifically bind one or more (e.g., three) antigen targets or target proteins, wherein a first polypeptide chain comprises a structure represented by the formula:

V_{D1}-L₁-V_{D2}-L₂-C_{L} [I]

and a second polypeptide chain comprises a structure represented by the formula:

V_{D3}-L₃-V_{D4}-L₄-C_{H1}-hinge-C_{H2}-C_{H3} [II]

and a third polypeptide chain comprises a structure represented by the formula:

V_{H3}-C_{H1}-hinge-C_{H2}-C_{H3} [III]

and a fourth polypeptide chain comprises a structure represented by the formula:

V_{L3}-C_{L} [IV]

wherein:
V_{D1} is a variable domain of heavy or light chain of a first immunoglobulin;
V_{D2} is a variable domain of heavy or light chain of a second immunoglobulin;
V_{D3} is a variable domain of heavy or light chain of a third immunoglobulin;
V_{D4} is a variable domain of heavy or light chain of a fourth immunoglobulin;
V_{H3} is an immunoglobulin heavy chain variable domain;
V_{L3} is an immunoglobulin light chain variable domain;
C_{L} is an immunoglobulin light chain constant domain;
C_{H1} is an immunoglobulin C_{H1} heavy chain constant domain;
C_{H2} is an immunoglobulin C_{H2} heavy chain constant domain;
C_{H3} is an immunoglobulin C_{H3} heavy chain constant domain;
hinge is an immunoglobulin hinge region connecting the C_{H1} and C_{H2} domains; and
L₁, L₂, L₃ and L₄ are amino acid linkers;
   and wherein the polypeptide of formula I and the polypeptide of formula II form a cross-over light chain-heavy chain pair.

In some embodiments, the binding protein of the disclosure comprises three antigen binding sites that specifically bind one, two, or three antigen targets or target proteins. In some embodiments, the binding protein binds three antigen targets. In some embodiments, the binding protein binds three different antigen targets. In some embodiments, two of the antigen binding sites bind the same antigen target. In those embodiments, the binding protein comprises the same binding domains twice, or different binding domains, and/or specifically binds different antigens or epitopes on the same antigen target. In some embodiments, three of the antigen binding sites bind the same antigen target. In those embodiments, the binding protein comprises the same binding domains three times, or different binding domains, and/or specifically binds different antigens or epitopes on the same antigen target.

In some embodiments, V_{L1}, V_{L2} and V_{L3} are each independently a variable domain derived from an amino acid sequence as set forth in any one of SEQ ID NOs: 2, 4, 10, 14, 18, 22 or 115; and V_{H1}, V_{H2} and V_{H3}, are each independently a variable domain derived from an amino acid sequence as set forth in any one of SEQ ID NOs: 1, 3, 9, 13, 17, 21 or 114. In other embodiments, V_{L1}, V_{L2} and V_{L3} are each independently a variable domain derived from an amino acid sequence as set forth in any one of SEQ ID NOs: 61, 63, 69, 71, 74, 76, 82, 86, 88 or 94; and V_{H1}, V_{H2} and V_{H3}, are each independently a variable domain derived from an amino acid sequence as set forth in any one of SEQ ID NOs: 60, 62, 68, 73, 75, 81, 85, 87 or 93. In other embodiments, V_{L1}, V_{L2} and V_{L3} each independently comprise light chain complementarity determining regions of a variable domain comprising an amino acid sequence as set forth in any one of SEQ ID NOs: 43-59, 123-125; and V_{H1}, V_{H2} and V_{H3} each independently comprise heavy chain complementarity determining regions of a variable domain comprising an amino acid sequence as set forth in any one of SEQ ID NOs: 25-42, 120-122. In other embodiments, V_{L1}, V_{L2} and V_{L3} each independently comprise light chain complementarity determining regions of a variable domain comprising an amino acid sequence as set forth in any one of SEQ ID NOs: 61, 63, 69, 71, 74, 76, 82, 86, 88 or 94; and V_{H1}, V_{H2} and V_{H3} each independently comprise heavy chain complementarity determining regions of a variable domain comprising an amino acid sequence as set forth in any one of SEQ ID NOs: 60, 62, 68, 73, 75, 81, 85, 87 or 93. In some embodiments, V_{H1}, V_{H2} and V_{H3} each independently comprise heavy chain complementarity determining regions and/or a variable domain sequence shown in Tables 2-5.

In some embodiments, V_{L1}, V_{L2} and V_{L3} each independently comprise a variable domain sequence as set forth in any one of SEQ ID NOs:169, 171, and 173; and/or V_{H1}, V_{H2}, and V_{H3} each independently comprise a variable domain sequence as set forth in any one of SEQ ID NOs:168, 170, and 172. In some embodiments, V_{L1}, V_{L2} and V_{L3} each independently comprise light chain complementarity determining regions comprising an amino acid sequence selected from the group consisting of SEQ ID NOs: 141-147, 178, and 179; and/or V_{H1}, V_{H2} and V_{H3} each independently comprise heavy chain complementarity determining regions comprising an amino acid sequence selected from the group consisting of SEQ ID NOs: 129-137. In some embodiments, V_{L1}, V_{L2} and V_{L3} each independently comprise a variable domain sequence as set forth in any one of SEQ ID NOs:151, 153, 155, 157, 159, 161, 163, 165, and 167; and/or V_{H1}, V_{H2}, and V_{H3} each independently comprise a variable domain sequence as set forth in any one of SEQ ID NOs:150, 152, 154, 156, 158, 160, 162, 164, and 166. In some embodiments, V_{L1}, V_{L2} and V_{L3} each independently comprise light chain complementarity determining regions comprising an amino acid sequence as set forth in any one of SEQ ID NOs: 43-59, 123-125, 138-140, and 149; and/or V_{H1}, V_{H2} and V_{H3} each independently comprise heavy chain complementarity determining regions of a variable domain comprising an amino acid sequence as set forth in any one of SEQ ID NOs: 25-42, 120-122, and 126-128. In some embodiments, V_{L1}, V_{L2} and V_{L3} each independently comprise light chain complementarity determining regions and/or a variable domain sequence shown in Tables 2-5.

In particular embodiments, the order of the V_{H1} and V_{H2} domains, and therefore the order of V_{L1} and V_{L2} domains, in the polypeptide chain of formula I or the polypeptide chain of formula II (i.e., to reverse the order) are swapped.

In some embodiments, the first polypeptide chain comprises the amino acid sequence of SEQ ID NO: 4 or an amino acid sequence that is at least 95% identical to the amino acid sequence of SEQ ID NO: 4 optionally comprising CDRs that are 100% identical to the CDRs of the polypeptide chain of SEQ ID NO: 4; the second polypeptide chain comprises the amino acid sequence of SEQ ID NO: 3 or an amino acid sequence that is at least 95% identical to the amino acid sequence of SEQ ID NO: 3 optionally comprising CDRs that are 100% identical to the CDRs of the polypeptide chain of SEQ ID NO: 3; the third polypeptide chain comprises the amino acid sequence of SEQ ID NO: 1 or an amino acid sequence that is at least 95% identical to the amino acid sequence of SEQ ID NO: 1 optionally comprising CDRs that are 100% identical to the CDRs of the polypeptide chain of SEQ ID NO: 1; and the fourth polypeptide chain comprises the amino acid sequence of SEQ ID NO: 2 or an amino acid sequence that is at least 95% identical to the amino acid sequence of SEQ ID NO: 2 optionally comprising CDRs that are 100% identical to the CDRs of the polypeptide chain of SEQ ID NO: 2.

In some embodiments, the first polypeptide chain comprises the amino acid sequence of SEQ ID NO: 10 or an amino acid sequence that is at least 95% identical to the amino acid sequence of SEQ ID NO: 10 optionally comprising CDRs that are 100% identical to the CDRs of the polypeptide chain of SEQ ID NO: 10; the second polypeptide chain comprises the amino acid sequence of SEQ ID NO: 9 or an amino acid sequence that is at least 95% identical to the amino acid sequence of SEQ ID NO: 9 optionally comprising CDRs that are 100% identical to the CDRs of the polypeptide chain of SEQ ID NO: 9; the third polypeptide chain comprises the amino acid sequence of SEQ ID NO: 1 or an amino acid sequence that is at least 95% identical to the amino acid sequence of SEQ ID NO: 1 optionally comprising CDRs that are 100% identical to the CDRs of the polypeptide chain of SEQ ID NO: 1; and the fourth polypeptide chain comprises the amino acid sequence of SEQ ID NO: 2 or an amino acid sequence that is at least 95% identical to the amino acid sequence of SEQ ID NO: 2 optionally comprising CDRs that are 100% identical to the CDRs of the polypeptide chain of SEQ ID NO: 2.

In some embodiments, the first polypeptide chain comprises the amino acid sequence of SEQ ID NO: 4 or an amino acid sequence that is at least 95% identical to the amino acid sequence of SEQ ID NO: 4 optionally comprising CDRs that are 100% identical to the CDRs of the polypeptide chain of SEQ ID NO: 4; the second polypeptide chain comprises the amino acid sequence of SEQ ID NO: 3 or an amino acid sequence that is at least 95% identical to the amino acid sequence of SEQ ID NO: 3 optionally comprising CDRs that are 100% identical to the CDRs of the polypeptide chain of SEQ ID NO: 3; the third polypeptide chain comprises the amino acid sequence of SEQ ID NO: 13 or an amino acid sequence that is at least 95% identical to the amino acid sequence of SEQ ID NO: 13 optionally comprising CDRs that are 100% identical to the CDRs of the polypeptide chain of SEQ ID NO: 13; and the fourth polypeptide chain comprises the amino acid sequence of SEQ ID NO: 14 or an amino acid sequence that is at least 95% identical to the amino acid sequence of SEQ ID NO: 14 optionally comprising CDRs that are 100% identical to the CDRs of the polypeptide chain of SEQ ID NO: 14.

In some embodiments, the first polypeptide chain comprises the amino acid sequence of SEQ ID NO: 10 or an amino acid sequence that is at least 95% identical to the amino acid sequence of SEQ ID NO: 10 optionally comprising CDRs that are 100% identical to the CDRs of the polypeptide chain of SEQ ID NO: 10; the second polypeptide chain comprises the amino acid sequence of SEQ ID NO: 9 or an amino acid sequence that is at least 95% identical to the amino acid sequence of SEQ ID NO: 9 optionally comprising CDRs that are 100% identical to the CDRs of the polypeptide chain of SEQ ID NO: 9; the third polypeptide chain comprises the amino acid sequence of SEQ ID NO: 13 or an amino acid sequence that is at least 95% identical to the amino acid sequence of SEQ ID NO: 13 optionally comprising CDRs that are 100% identical to the CDRs of the polypeptide chain of SEQ ID NO: 13; and the fourth polypeptide chain comprises the amino acid sequence of SEQ ID NO: 14 or an amino acid sequence that is at least 95% identical to the amino acid sequence of SEQ ID NO: 14 optionally comprising CDRs that are 100% identical to the CDRs of the polypeptide chain of SEQ ID NO: 14.

In some embodiments, the first polypeptide chain comprises the amino acid sequence of SEQ ID NO: 4 or an amino acid sequence that is at least 95% identical to the amino acid sequence of SEQ ID NO: 4 optionally comprising CDRs that are 100% identical to the CDRs of the polypeptide chain of SEQ ID NO: 4; the second polypeptide chain comprises the amino acid sequence of SEQ ID NO: 3 or an amino acid sequence that is at least 95% identical to the amino acid sequence of SEQ ID NO: 3 optionally comprising CDRs that are 100% identical to the CDRs of the polypeptide chain of SEQ ID NO: 3; the third polypeptide chain comprises the amino acid sequence of SEQ ID NO: 17 or an amino acid sequence that is at least 95% identical to the amino acid sequence of SEQ ID NO: 17 optionally comprising CDRs that are 100% identical to the CDRs of the polypeptide chain of SEQ ID NO: 17; and the fourth polypeptide chain comprises the amino acid sequence of SEQ ID NO: 18 or an amino acid sequence that is at least 95% identical to the amino acid sequence of SEQ ID NO: 18 optionally comprising CDRs that are 100% identical to the CDRs of the polypeptide chain of SEQ ID NO: 18.

In some embodiments, the first polypeptide chain comprises the amino acid sequence of SEQ ID NO: 10 or an amino acid sequence that is at least 95% identical to the amino acid sequence of SEQ ID NO: 10 optionally comprising CDRs that are 100% identical to the CDRs of the polypeptide chain of SEQ ID NO: 10; the second polypeptide chain comprises the amino acid sequence of SEQ ID NO: 9 or an amino acid sequence that is at least 95% identical to the amino acid sequence of SEQ ID NO: 9 optionally comprising CDRs that are 100% identical to the CDRs of the polypeptide chain of SEQ ID NO: 9; the third polypeptide chain comprises the amino acid sequence of SEQ ID NO: 17 or an amino acid sequence that is at least 95% identical to the amino acid sequence of SEQ ID NO: 17 optionally comprising CDRs that are 100% identical to the CDRs of the polypeptide chain of SEQ ID NO: 17; and the fourth polypeptide chain comprises the amino acid sequence of SEQ ID NO: 18 or an amino acid sequence that is at least 95% identical to the amino acid sequence of SEQ ID NO: 18 optionally comprising CDRs that are 100% identical to the CDRs of the polypeptide chain of SEQ ID NO: 18.

In some embodiments, the first polypeptide chain comprises the amino acid sequence of SEQ ID NO: 4 or an amino acid sequence that is at least 95% identical to the amino acid sequence of SEQ ID NO: 4 optionally comprising CDRs that are 100% identical to the CDRs of the polypeptide chain of SEQ ID NO: 4; the second polypeptide chain comprises the amino acid sequence of SEQ ID NO: 3 or an amino acid sequence that is at least 95% identical to the amino acid sequence of SEQ ID NO: 3 optionally comprising CDRs that are 100% identical to the CDRs of the polypeptide chain of SEQ ID NO: 3; the third polypeptide chain comprises the amino acid sequence of SEQ ID NO: 21 or an amino acid sequence that is at least 95% identical to the amino acid sequence of SEQ ID NO: 21 optionally comprising CDRs that are 100% identical to the CDRs of the polypeptide chain of SEQ ID NO: 21; and the fourth polypeptide chain comprises the amino acid sequence of SEQ ID NO: 22 or an amino acid sequence that is at least 95% identical to the amino acid sequence of SEQ ID NO: 22 optionally comprising CDRs that are 100% identical to the CDRs of the polypeptide chain of SEQ ID NO: 22.

In some embodiments, the first polypeptide chain comprises the amino acid sequence of SEQ ID NO: 10 or an amino acid sequence that is at least 95% identical to the amino acid sequence of SEQ ID NO: 10 optionally comprising CDRs that are 100% identical to the CDRs of the polypeptide chain of SEQ ID NO: 10; the second polypeptide chain comprises the amino acid sequence of SEQ ID NO: 9 or an amino acid sequence that is at least 95% identical to the amino acid sequence of SEQ ID NO: 9 optionally comprising CDRs that are 100% identical to the CDRs of the polypeptide chain of SEQ ID NO: 9; the third polypeptide chain comprises the amino acid sequence of SEQ ID NO: 21 or an amino acid sequence that is at least 95% identical to the amino acid sequence of SEQ ID NO: 21 optionally comprising CDRs that are 100% identical to the CDRs of the polypeptide chain of SEQ ID NO: 21; and the fourth polypeptide chain comprises the amino acid sequence of SEQ ID NO: 22 or an amino acid sequence that is at least 95% identical to the amino acid sequence of SEQ ID NO: 22 optionally comprising CDRs that are 100% identical to the CDRs of the polypeptide chain of SEQ ID NO: 22.

In some embodiments, the first polypeptide chain comprises the amino acid sequence of SEQ ID NO: 63 or an amino acid sequence that is at least 95% identical to the amino acid sequence of SEQ ID NO: 63 optionally comprising CDRs that are 100% identical to the CDRs of the polypeptide chain of SEQ ID NO: 63; the second polypeptide chain comprises the amino acid sequence of SEQ ID NO: 62 or an amino acid sequence that is at least 95% identical to the amino acid sequence of SEQ ID NO: 62 optionally comprising CDRs that are 100% identical to the CDRs of the polypeptide chain of SEQ ID NO: 62; the third polypeptide chain comprises the amino acid sequence of SEQ ID NO: 60 or an amino acid sequence that is at least 95% identical to the amino acid sequence of SEQ ID NO: 60 optionally comprising CDRs that are 100% identical to the CDRs of the polypeptide chain of SEQ ID NO: 60; and the fourth polypeptide chain comprises the amino acid sequence of SEQ ID NO: 61 or an amino acid sequence that is at least 95% identical to the amino acid sequence of SEQ ID NO: 61 optionally comprising CDRs that are 100% identical to the CDRs of the polypeptide chain of SEQ ID NO: 61.

In some embodiments, the first polypeptide chain comprises the amino acid sequence of SEQ ID NO: 69 or an amino acid sequence that is at least 95% identical to the amino acid sequence of SEQ ID NO: 69 optionally comprising CDRs that are 100% identical to the CDRs of the polypeptide chain of SEQ ID NO: 69; the second polypeptide chain comprises the amino acid sequence of SEQ ID NO: 68 or an amino acid sequence that is at least 95% identical to the amino acid sequence of SEQ ID NO: 68 optionally comprising CDRs that are 100% identical to the CDRs of the polypeptide chain of SEQ ID NO: 68; the third polypeptide chain comprises the amino acid sequence of SEQ ID NO: 60 or an amino acid sequence that is at least 95% identical to the amino acid sequence of SEQ ID NO: 60 optionally comprising CDRs that are 100% identical to the CDRs of the polypeptide chain of SEQ ID NO: 60; and the fourth polypeptide chain comprises the amino acid sequence of SEQ ID NO: 61 or an amino acid sequence that is at least 95% identical to the amino acid sequence of SEQ ID NO: 61 optionally comprising CDRs that are 100% identical to the CDRs of the polypeptide chain of SEQ ID NO: 61.

In some embodiments, the first polypeptide chain comprises the amino acid sequence of SEQ ID NO: 69 or an amino acid sequence that is at least 95% identical to the amino acid sequence of SEQ ID NO: 69 optionally comprising CDRs that are 100% identical to the CDRs of the polypeptide chain of SEQ ID NO: 69; the second polypeptide chain comprises the amino acid sequence of SEQ ID NO: 68 or an amino acid sequence that is at least 95% identical to the amino acid sequence of SEQ ID NO: 68 optionally comprising CDRs that are 100% identical to the CDRs of the polypeptide chain of SEQ ID NO: 68; the third polypeptide chain comprises the amino acid sequence of SEQ ID NO: 60 or an amino acid sequence that is at least 95% identical to the amino acid sequence of SEQ ID NO: 60 optionally comprising CDRs that are 100% identical to the CDRs of the polypeptide chain of SEQ ID NO: 60; and the fourth polypeptide chain comprises the amino acid sequence of SEQ ID NO: 71 or an amino acid sequence that is at least 95% identical to the amino acid sequence of SEQ ID NO: 71 optionally comprising CDRs that are 100% identical to the CDRs of the polypeptide chain of SEQ ID NO: 71.

In some embodiments, the first polypeptide chain comprises the amino acid sequence of SEQ ID NO: 76 or an amino acid sequence that is at least 95% identical to the amino acid sequence of SEQ ID NO: 76 optionally comprising CDRs that are 100% identical to the CDRs of the polypeptide chain of SEQ ID NO: 76; the second polypeptide chain comprises the amino acid sequence of SEQ ID NO: 75 or an amino acid sequence that is at least 95% identical to the amino acid sequence of SEQ ID NO: 75 optionally comprising CDRs that are 100% identical to the CDRs of the polypeptide chain of SEQ ID NO: 75; the third polypeptide chain comprises the amino acid sequence of SEQ ID NO: 73 or an amino acid sequence that is at least 95% identical to the amino acid sequence of SEQ ID NO: 73 optionally comprising CDRs that are 100% identical to the CDRs of the polypeptide chain of SEQ ID NO: 73; and the fourth polypeptide chain comprises the amino acid sequence of SEQ ID NO: 74 or an amino acid sequence that is at least 95% identical to the amino acid sequence of SEQ ID NO: 74 optionally comprising CDRs that are 100% identical to the CDRs of the polypeptide chain of SEQ ID NO: 74.

In some embodiments, the first polypeptide chain comprises the amino acid sequence of SEQ ID NO: 82 or an amino acid sequence that is at least 95% identical to the amino acid sequence of SEQ ID NO: 82 optionally comprising CDRs that are 100% identical to the CDRs of the polypeptide chain of SEQ ID NO: 82; the second polypeptide chain comprises the amino acid sequence of SEQ ID NO: 81 or an amino acid sequence that is at least 95% identical to the amino acid sequence of SEQ ID NO:81 optionally comprising CDRs that are 100% identical to the CDRs of the polypeptide chain of SEQ ID NO: 81; the third polypeptide chain comprises the amino acid sequence of SEQ ID NO: 73 or an amino acid sequence that is at least 95% identical to the amino acid sequence of SEQ ID NO: 73 optionally comprising CDRs that are 100% identical to the CDRs of the polypeptide chain of SEQ ID NO: 73; and the fourth polypeptide chain comprises the amino acid sequence of SEQ ID NO: 74 or an amino acid sequence that is at least 95% identical to the amino acid sequence of SEQ ID NO: 74 optionally comprising CDRs that are 100% identical to the CDRs of the polypeptide chain of SEQ ID NO: 74.

In some embodiments, the first polypeptide chain comprises the amino acid sequence of SEQ ID NO: 88 or an amino acid sequence that is at least 95% identical to the amino acid sequence of SEQ ID NO: 88 optionally comprising CDRs that are 100% identical to the CDRs of the polypeptide chain of SEQ ID NO: 88; the second polypeptide chain comprises the amino acid sequence of SEQ ID NO: 87 or an amino acid sequence that is at least 95% identical to the amino acid sequence of SEQ ID NO: 87 optionally comprising CDRs that are 100% identical to the CDRs of the polypeptide chain of SEQ ID NO: 87; the third polypeptide chain comprises the amino acid sequence of SEQ ID NO: 85 or an amino acid sequence that is at least 95% identical to the amino acid sequence of SEQ ID NO: 85 optionally comprising CDRs that are 100% identical to the CDRs of the polypeptide chain of SEQ ID NO: 85; and the fourth polypeptide chain comprises the amino acid sequence of SEQ ID NO: 86 or an amino acid sequence that is at least 95% identical to the amino acid sequence of SEQ ID NO: 86 optionally comprising CDRs that are 100% identical to the CDRs of the polypeptide chain of SEQ ID NO: 86.

In some embodiments, the first polypeptide chain comprises the amino acid sequence of SEQ ID NO: 94 or an amino acid sequence that is at least 95% identical to the amino acid sequence of SEQ ID NO: 94 optionally comprising CDRs that are 100% identical to the CDRs of the polypeptide chain of SEQ ID NO: 94; the second polypeptide chain comprises the amino acid sequence of SEQ ID NO: 93 or an amino acid sequence that is at least 95% identical to the amino acid sequence of SEQ ID NO: 93 optionally comprising CDRs that are 100% identical to the CDRs of the polypeptide chain of SEQ ID NO: 93; the third polypeptide chain comprises the amino acid sequence of SEQ ID NO: 85 or an amino acid sequence that is at least 95% identical to the amino acid sequence of SEQ ID NO: 85 optionally comprising CDRs that are 100% identical to the CDRs of the polypeptide chain of SEQ ID NO: 85; and the fourth polypeptide chain comprises the amino acid sequence of SEQ ID NO: 86 or an amino acid sequence that is at least 95% identical to the amino acid sequence of SEQ ID NO: 86 optionally comprising CDRs that are 100% identical to the CDRs of the polypeptide chain of SEQ ID NO: 86.

In some embodiments, the first polypeptide chain comprises the amino acid sequence of SEQ ID NO: 69 or an amino acid sequence that is at least 95% identical to the amino acid sequence of SEQ ID NO: 69 optionally comprising CDRs that are 100% identical to the CDRs of the polypeptide chain of SEQ ID NO: 69; the second polypeptide chain comprises the amino acid sequence of SEQ ID NO: 68 or an amino acid sequence that is at least 95% identical to the amino acid sequence of SEQ ID NO: 68 optionally comprising CDRs that are 100% identical to the CDRs of the polypeptide chain of SEQ ID NO: 68; the third polypeptide chain comprises the amino acid sequence of SEQ ID NO: 73 or an amino acid sequence that is at least 95% identical to the amino acid sequence of SEQ ID NO: 73 optionally comprising CDRs that are 100% identical to the CDRs of the polypeptide chain of SEQ ID NO: 73; and the fourth polypeptide chain comprises the amino acid sequence of SEQ ID NO: 74 or an amino acid sequence that is at least 95% identical to the amino acid sequence of SEQ ID NO: 74 optionally comprising CDRs that are 100% identical to the CDRs of the polypeptide chain of SEQ ID NO: 74.

In some embodiments, the first polypeptide chain comprises the amino acid sequence of SEQ ID NO: 69 or an amino acid sequence that is at least 95% identical to the amino acid sequence of SEQ ID NO: 69 optionally comprising CDRs that are 100% identical to the CDRs of the polypeptide chain of SEQ ID NO: 69; the second polypeptide chain comprises the amino acid sequence of SEQ ID NO: 68 or an amino acid sequence that is at least 95% identical to the amino acid sequence of SEQ ID NO: 68 optionally comprising CDRs that are 100% identical to the CDRs of the polypeptide chain of SEQ ID NO: 68; the third polypeptide chain comprises the amino acid sequence of SEQ ID NO: 85 or an amino acid sequence that is at least 95% identical to the amino acid sequence of SEQ ID NO: 85 optionally comprising CDRs that are 100% identical to the CDRs of the polypeptide chain of SEQ ID NO: 85; and the fourth polypeptide chain comprises the amino acid sequence of SEQ ID NO: 86 or an amino acid sequence that is at least 95% identical to the amino acid sequence of SEQ ID NO: 86 optionally comprising CDRs that are 100% identical to the CDRs of the polypeptide chain of SEQ ID NO: 86.

In some embodiments, the first polypeptide chain comprises the amino acid sequence of SEQ ID NO: 63 or an amino acid sequence that is at least 95% identical to the amino acid sequence of SEQ ID NO: 63 optionally comprising CDRs that are 100% identical to the CDRs of the polypeptide chain of SEQ ID NO: 63; the second polypeptide chain comprises the amino acid sequence of SEQ ID NO: 62 or an amino acid sequence that is at least 95% identical to the amino acid sequence of SEQ ID NO: 62 optionally comprising CDRs that are 100% identical to the CDRs of the polypeptide chain of SEQ ID NO: 62; the third polypeptide chain comprises the amino acid sequence of SEQ ID NO: 73 or an amino acid sequence that is at least 95% identical to the amino acid sequence of SEQ ID NO: 73 optionally comprising CDRs that are 100% identical to the CDRs of the polypeptide chain of SEQ ID NO: 73; and the fourth polypeptide chain comprises the amino acid sequence of SEQ ID NO: 74 or an amino acid sequence that is at least 95% identical to the amino acid sequence of SEQ ID NO: 74 optionally comprising CDRs that are 100% identical to the CDRs of the polypeptide chain of SEQ ID NO: 74.

In some embodiments, the first polypeptide chain comprises the amino acid sequence of SEQ ID NO: 63 or an amino acid sequence that is at least 95% identical to the amino acid sequence of SEQ ID NO: 63 optionally comprising CDRs that are 100% identical to the CDRs of the polypeptide chain of SEQ ID NO: 63; the second polypeptide chain comprises the amino acid sequence of SEQ ID NO: 62 or an amino acid sequence that is at least 95% identical to the amino acid sequence of SEQ ID NO: 62 optionally comprising CDRs that are 100% identical to the CDRs of the polypeptide chain of SEQ ID NO: 62; the third polypeptide chain comprises the amino acid sequence of SEQ ID NO: 85 or an amino acid sequence that is at least 95% identical to the amino acid sequence of SEQ ID NO: 85 optionally comprising CDRs that are 100% identical to the CDRs of the polypeptide chain of SEQ ID NO: 85; and the fourth polypeptide chain comprises the amino acid sequence of SEQ ID NO: 86 or an amino acid sequence that is at least 95% identical to the amino acid sequence of SEQ ID NO: 86 optionally comprising CDRs that are 100% identical to the CDRs of the polypeptide chain of SEQ ID NO: 86.

In some embodiments, the first polypeptide chain comprises the amino acid sequence of SEQ ID NO: 4 or an amino acid sequence that is at least 95% identical to the amino acid sequence of SEQ ID NO: 4 optionally comprising CDRs that are 100% identical to the CDRs of the polypeptide chain of SEQ ID NO: 4; the second polypeptide chain comprises the amino acid sequence of SEQ ID NO: 3 or an amino acid sequence that is at least 95% identical to the amino acid sequence of SEQ ID NO: 3 optionally comprising CDRs that are 100% identical to the CDRs of the polypeptide chain of SEQ ID NO: 3; the third polypeptide chain comprises the amino acid sequence of SEQ ID NO: 114 or an amino acid sequence that is at least 95% identical to the amino acid sequence of SEQ ID NO: 114 optionally comprising CDRs that are 100% identical to the CDRs of the polypeptide chain of SEQ ID NO: 114; and the fourth polypeptide chain comprises the amino acid sequence of SEQ ID NO: 115 or an amino acid sequence that is at least 95% identical to the amino acid sequence of SEQ ID NO: 115 optionally comprising CDRs that are 100% identical to the CDRs of the polypeptide chain of SEQ ID NO: 115.

In some embodiments, the first polypeptide chain comprises the amino acid sequence of SEQ ID NO: 10 or an amino acid sequence that is at least 95% identical to the amino acid sequence of SEQ ID NO: 10 optionally comprising CDRs that are 100% identical to the CDRs of the polypeptide chain of SEQ ID NO: 10; the second polypeptide chain comprises the amino acid sequence of SEQ ID NO: 9 or an amino acid sequence that is at least 95% identical to the amino acid sequence of SEQ ID NO: 9 optionally comprising CDRs that are 100% identical to the CDRs of the polypeptide chain of SEQ ID NO: 9; the third polypeptide chain comprises the amino acid sequence of SEQ ID NO: 114 or an amino acid sequence that is at least 95% identical to the amino acid sequence of SEQ ID NO: 114 optionally comprising CDRs that are 100% identical to the CDRs of the polypeptide chain of SEQ ID NO: 114; and the fourth polypeptide chain comprises the amino acid sequence of SEQ ID NO: 115 or an amino acid sequence that is at least 95% identical to the amino acid sequence of SEQ ID NO: 115 optionally comprising CDRs that are 100% identical to the CDRs of the polypeptide chain of SEQ ID NO: 115.

In other embodiments, the binding protein of the disclosure is a trispecific and/or trivalent binding protein comprising four polypeptide chains that form three antigen binding sites that specifically bind one or more (e.g., three) different target proteins, wherein a first polypeptide chain has a structure represented by the formula:

V_{L2}-L₁-V_{L1}-L₂-C_{L} [I]

and a second polypeptide chain has a structure represented by the formula:

V_{H1}-L₃-V_{H2}-L₄-C_{H1}-hinge-C_{H2}-C_{H3}(hole) [II]

and a third polypeptide chain has a structure represented by the formula:

V_{H3}-C_{H1}-hinge-C_{H2}-C_{H3}(knob) [III]

and a fourth polypeptide chain has a structure represented by the formula:

V_{L3}-C_{L} [IV]

wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain; and
L₁, L₂, L₃ and L₄ are amino acid linkers;
   and wherein the polypeptide of formula I and the polypeptide of formula II form a cross-over light chain-heavy chain pair.

In other embodiments, the binding protein of the disclosure is a trispecific and/or trivalent binding protein comprising four polypeptide chains that form three antigen binding sites that specifically bind one or more (e.g., three) target proteins, wherein a first polypeptide chain has a structure represented by the formula:

V_{L2}-L₁-V_{L1}-L₂-C_{L} [I]

and a second polypeptide chain has a structure represented by the formula:

V_{H1}-L₃-V_{H2}-L₄-C_{H1}-hinge-C_{H2}-C_{H3}(hole) [II]

and a third polypeptide chain has a structure represented by the formula:

V_{H3}-C_{H1}-hinge-C_{H2}-C_{H3}(knob) [III]

and a fourth polypeptide chain has a structure represented by the formula:

V_{L3}-C_{L} [IV]

wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain; and
L₁, L₂, L₃ and L₄ are amino acid linkers;
   and wherein the polypeptide of formula I and the polypeptide of formula II form a cross-over light chain-heavy chain pair.

In some embodiments, the first polypeptide chain and the second polypeptide chain have a cross-over orientation that forms two distinct antigen binding sites. In some embodiments, the VH1 and VL1 form a binding pair and form the first antigen binding site. In some embodiments, the VH2 and VL2 form a binding pair and form the second antigen binding site. In some embodiments, the third polypeptide and the fourth polypeptide form a third antigen binding site. In some embodiments, the VH3 and VL3 form a binding pair and form the third antigen binding site. In some embodiments, the second polypeptide chain and the third polypeptide chain comprise one or more modifications. In some embodiments, the second polypeptide chain and the third polypeptide chain of a binding protein are different, e.g., having different C_{H1}, C_{H2}, and/or C_{H3} domain(s) (such as those including a modification described herein). In some embodiments, the first polypeptide chain and the fourth polypeptide chain comprise one or more modifications. In some embodiments, the first polypeptide chain and the fourth polypeptide chain of a binding protein are different, e.g., having different C_{L} domains (such as those including a modification described herein, and/or lambda vs. kapp C_{L} domains).

In some embodiments, a binding protein of the present disclosure comprises an antigen binding site comprising a heavy chain variable domain comprising an amino acid sequence that is at least 95%, at least 96%, at least 97%, at least 98%, at least 99%, or 100% identical to SEQ ID NO:150, optionally comprising CDRs that are 100% identical to the CDRs of the polypeptide chain of SEQ ID NO: 150, and/or a light chain variable domain comprising an amino acid sequence that is at least 95%, at least 96%, at least 97%, at least 98%, at least 99%, or 100% identical to SEQ ID NO:151, optionally comprising CDRs that are 100% identical to the CDRs of the polypeptide chain of SEQ ID NO: 151. In some embodiments, a binding protein of the present disclosure comprises an antigen binding site comprising a heavy chain variable domain comprising an amino acid sequence that is at least 95%, at least 96%, at least 97%, at least 98%, at least 99%, or 100% identical to SEQ ID NO:152, optionally comprising CDRs that are 100% identical to the CDRs of the polypeptide chain of SEQ ID NO: 152, and/or a light chain variable domain comprising an amino acid sequence that is at least 95%, at least 96%, at least 97%, at least 98%, at least 99%, or 100% identical to SEQ ID NO:153, optionally comprising CDRs that are 100% identical to the CDRs of the polypeptide chain of SEQ ID NO: 153. In some embodiments, a binding protein of the present disclosure comprises an antigen binding site comprising a heavy chain variable domain comprising an amino acid sequence that is at least 95%, at least 96%, at least 97%, at least 98%, at least 99%, or 100% identical to SEQ ID NO:154, optionally comprising CDRs that are 100% identical to the CDRs of the polypeptide chain of SEQ ID NO: 154, and/or a light chain variable domain comprising an amino acid sequence that is at least 95%, at least 96%, at least 97%, at least 98%, at least 99%, or 100% identical to SEQ ID NO:155, optionally comprising CDRs that are 100% identical to the CDRs of the polypeptide chain of SEQ ID NO: 155. In some embodiments, a binding protein of the present disclosure comprises an antigen binding site comprising a heavy chain variable domain comprising an amino acid sequence that is at least 95%, at least 96%, at least 97%, at least 98%, at least 99%, or 100% identical to SEQ ID NO:156, optionally comprising CDRs that are 100% identical to the CDRs of the polypeptide chain of SEQ ID NO: 156, and/or a light chain variable domain comprising an amino acid sequence that is at least 95%, at least 96%, at least 97%, at least 98%, at least 99%, or 100% identical to SEQ ID NO:157, optionally comprising CDRs that are 100% identical to the CDRs of the polypeptide chain of SEQ ID NO: 157. In some embodiments, a binding protein of the present disclosure comprises an antigen binding site comprising a heavy chain variable domain comprising an amino acid sequence that is at least 95%, at least 96%, at least 97%, at least 98%, at least 99%, or 100% identical to SEQ ID NO:158, optionally comprising CDRs that are 100% identical to the CDRs of the polypeptide chain of SEQ ID NO: 158, and/or a light chain variable domain comprising an amino acid sequence that is at least 95%, at least 96%, at least 97%, at least 98%, at least 99%, or 100% identical to SEQ ID NO:159, optionally comprising CDRs that are 100% identical to the CDRs of the polypeptide chain of SEQ ID NO: 159. In some embodiments, a binding protein of the present disclosure comprises an antigen binding site comprising a heavy chain variable domain comprising an amino acid sequence that is at least 95%, at least 96%, at least 97%, at least 98%, at least 99%, or 100% identical to SEQ ID NO:160, optionally comprising CDRs that are 100% identical to the CDRs of the polypeptide chain of SEQ ID NO: 160, and/or a light chain variable domain comprising an amino acid sequence that is at least 95%, at least 96%, at least 97%, at least 98%, at least 99%, or 100% identical to SEQ ID NO:161, optionally comprising CDRs that are 100% identical to the CDRs of the polypeptide chain of SEQ ID NO: 161. In some embodiments, a binding protein of the present disclosure comprises an antigen binding site comprising a heavy chain variable domain comprising an amino acid sequence that is at least 95%, at least 96%, at least 97%, at least 98%, at least 99%, or 100% identical to SEQ ID NO:162, optionally comprising CDRs that are 100% identical to the CDRs of the polypeptide chain of SEQ ID NO: 162, and/or a light chain variable domain comprising an amino acid sequence that is at least 95%, at least 96%, at least 97%, at least 98%, at least 99%, or 100% identical to SEQ ID NO:163, optionally comprising CDRs that are 100% identical to the CDRs of the polypeptide chain of SEQ ID NO: 163. In some embodiments, a binding protein of the present disclosure comprises an antigen binding site comprising a heavy chain variable domain comprising an amino acid sequence that is at least 95%, at least 96%, at least 97%, at least 98%, at least 99%, or 100% identical to SEQ ID NO:164, optionally comprising CDRs that are 100% identical to the CDRs of the polypeptide chain of SEQ ID NO: 164, and/or a light chain variable domain comprising an amino acid sequence that is at least 95%, at least 96%, at least 97%, at least 98%, at least 99%, or 100% identical to SEQ ID NO:165, optionally comprising CDRs that are 100% identical to the CDRs of the polypeptide chain of SEQ ID NO: 165. In some embodiments, a binding protein of the present disclosure comprises an antigen binding site comprising a heavy chain variable domain comprising an amino acid sequence that is at least 95%, at least 96%, at least 97%, at least 98%, at least 99%, or 100% identical to SEQ ID NO:166, optionally comprising CDRs that are 100% identical to the CDRs of the polypeptide chain of SEQ ID NO: 166, and/or a light chain variable domain comprising an amino acid sequence that is at least 95%, at least 96%, at least 97%, at least 98%, at least 99%, or 100% identical to SEQ ID NO:167, optionally comprising CDRs that are 100% identical to the CDRs of the polypeptide chain of SEQ ID NO: 167. In some embodiments, a binding protein of the present disclosure comprises an antigen binding site comprising a heavy chain variable domain comprising an amino acid sequence that is at least 95%, at least 96%, at least 97%, at least 98%, at least 99%, or 100% identical to SEQ ID NO:168, optionally comprising CDRs that are 100% identical to the CDRs of the polypeptide chain of SEQ ID NO: 168, and/or a light chain variable domain comprising an amino acid sequence that is at least 95%, at least 96%, at least 97%, at least 98%, at least 99%, or 100% identical to SEQ ID NO:169, optionally comprising CDRs that are 100% identical to the CDRs of the polypeptide chain of SEQ ID NO: 169. In some embodiments, a binding protein of the present disclosure comprises an antigen binding site comprising a heavy chain variable domain comprising an amino acid sequence that is at least 95%, at least 96%, at least 97%, at least 98%, at least 99%, or 100% identical to SEQ ID NO:170, optionally comprising CDRs that are 100% identical to the CDRs of the polypeptide chain of SEQ ID NO: 170, and/or a light chain variable domain comprising an amino acid sequence that is at least 95%, at least 96%, at least 97%, at least 98%, at least 99%, or 100% identical to SEQ ID NO:171, optionally comprising CDRs that are 100% identical to the CDRs of the polypeptide chain of SEQ ID NO: 171. In some embodiments, a binding protein of the present disclosure comprises an antigen binding site comprising a heavy chain variable domain comprising an amino acid sequence that is at least 95%, at least 96%, at least 97%, at least 98%, at least 99%, or 100% identical to SEQ ID NO:172, optionally comprising CDRs that are 100% identical to the CDRs of the polypeptide chain of SEQ ID NO: 172, and/or a light chain variable domain comprising an amino acid sequence that is at least 95%, at least 96%, at least 97%, at least 98%, at least 99%, or 100% identical to SEQ ID NO:173, optionally comprising CDRs that are 100% identical to the CDRs of the polypeptide chain of SEQ ID NO: 173.

In some embodiments, a binding protein of the present disclosure binds to one, two, or three antigen targets with an equilibrium dissociation constant (K_{D}) that is less than or equal to 1 μM, 500 nM, 100 nM, 50 nM, 10 nM, 5 nM, or 1 nM. Exemplary assays for determining K_{D} are known in the art. For example, in some embodiments, K_{D} is determined by measuring binding kinetics at between 0° C. and 37° C. e.g., at 0° C., 4° C., 25° C., or 37° C.) using the techniques described in Example 1 (e.g., SPR or ELISA).

In some embodiments, a binding protein of the present disclosure activates CD4 and/or CD8 T cells in vitro and/or induces antibody-mediated in vitro cell killing of a cell expressing one or more antigen targets of one or more binding domains of the binding protein. Exemplary in vitro cell killing and T cell activation assays are known in the art. For example, in some embodiments, in vitro cell killing and/or T cell activation is assayed using the techniques described in Example 1.

In some embodiments, a binding protein of the present disclosure specifically binds to, and/or blocks signaling mediated by, one or more cytokines. Exemplary cytokine release assays are known in the art. For example, in some embodiments, cytokine release is assayed using the techniques described in Example 1.

In some embodiments, a binding protein of the present disclosure comprises a first antigen binding site that specifically binds a target protein on T cells, a second antigen binding site that specifically binds a target protein on T cells, and a third antigen binding site that specifically binds an antigen target or target protein. In some embodiments, a binding protein of the present disclosure comprises a first antigen binding site that specifically binds a target protein on T cells, a second antigen binding site that specifically binds a target protein on T cells, and a third antigen binding site that specifically binds a tumor target protein. In some embodiments, a binding protein of the present disclosure comprises a first antigen binding site that specifically binds a target protein on T cells, a second antigen binding site that specifically binds a target protein on T cells, and a third antigen binding site that specifically binds a human tumor target protein. In some embodiments, the first and second antigen binding sites specifically bind a tumor target protein for instance selected from CD3 and CD28, respectively. In some some embodiments, the first and second antigen binding sites specifically bind a tumor target protein for instance selected from CD28 and CD3, respectively. In some embodiments, the third antigen binding site specifically binds CD19, CD20, CD38, Her2, or LAMP1. Further examples of such targets and target proteins are provided infra.

In some embodiments, a binding protein of the present disclosure comprises a first antigen binding site that specifically binds CD3, a second antigen binding site that specifically binds CD28, and a third antigen binding site that specifically binds an antigen target or target protein. In some embodiments, a binding protein of the present disclosure comprises a first antigen binding site that specifically binds CD28, a second antigen binding site that specifically binds CD3, and a third antigen binding site that specifically binds an antigen target or target protein. Further examples of such antigen targets or target proteins are provided infra. In some embodiments, a binding protein of the present disclosure comprises a first antigen binding site that specifically binds CD3, a second antigen binding site that specifically binds CD28, and a third antigen binding site that specifically binds a tumor target protein. In some embodiments, a binding protein of the present disclosure comprises a first antigen binding site that specifically binds human CD3, a second antigen binding site that specifically binds human CD28, and a third antigen binding site that specifically binds a human tumor target protein. In some embodiments, a binding protein of the present disclosure comprises a first antigen binding site that specifically binds CD28, a second antigen binding site that specifically binds CD3, and a third antigen binding site that specifically binds a tumor target protein. In some embodiments, a binding protein of the present disclosure comprises a first antigen binding site that specifically binds human CD28, a second antigen binding site that specifically binds human CD3, and a third antigen binding site that specifically binds a human tumor target protein. In some embodiments, the third antigen binding site specifically binds CD19, CD20, CD38, Her2, or LAMP1. Further examples of such tumor antigen targets or tumor target proteins are provided infra.

In some embodiments, the antigen binding site that specifically binds CD3 comprises a heavy chain variable domain comprising the amino acid sequence of SEQ ID NO: 152 and a light chain variable domain comprising the amino acid sequence of SEQ ID NO: 153; or a heavy chain variable domain comprising the amino acid sequence of SEQ ID NO: 154 and a light chain variable domain comprising the amino acid sequence of SEQ ID NO: 155. Additional VH, VL, and/or CDR sequences of antibodies that specifically bind CD3 suitable for use in any of the binding proteins described herein may be found in International Publication No. WO2016/116626, which is incorporated by reference herein in its entirety. In some embodiments, the antigen binding site that specifically binds CD3 comprises six CDRs, or a heavy chain and a light chain variable domain, shown in Tables 2-5. In some embodiments, the antigen binding site that specifically binds CD3 comprises (i) three heavy chain CDRs of SEQ ID Nos. 34, 35 and 36, respectively, and three light chain CDRs of SEQ ID Nos. 52, 53 and 54, respectively; or (ii) three heavy chain CDRs of SEQ ID Nos. 34, 35 and 36, respectively, and three light chain CDRs of SEQ ID Nos. 149, 53 and 54, respectively. In some embodiments, the antigen binding site that specifically binds CD3 is part of a polypeptide chain comprising the amino acid sequence of SEQ ID NO:3 or an amino acid sequence that is at least 95% identical to the amino acid sequence of SEQ ID NO:3 optionally comprising CDRs that are 100% identical to the CDRs of the polypeptide chain of SEQ ID NO:3. In some embodiments, the antigen binding site that specifically binds CD3 is part of a polypeptide chain comprising the amino acid sequence of SEQ ID NO:4 or an amino acid sequence that is at least 95% identical to the amino acid sequence of SEQ ID NO:4 optionally comprising CDRs that are 100% identical to the CDRs of the polypeptide chain of SEQ ID NO:4.

In some embodiments, the antigen binding site that specifically binds CD28 comprises a heavy chain variable domain comprising the amino acid sequence of SEQ ID NO: 160 and a light chain variable domain comprising the amino acid sequence of SEQ ID NO: 161; or a heavy chain variable domain comprising the amino acid sequence of SEQ ID NO: 162 and a light chain variable domain comprising the amino acid sequence of SEQ ID NO: 163. In some embodiments, the antigen binding site that specifically binds CD28 comprises six CDRs, or a heavy chain and a light chain variable domain, shown in Tables 2-5. In some embodiments, the antigen binding site that specifically binds CD28 comprises (i) three heavy chain CDRs of SEQ ID Nos. 28, 29 and 30, respectively, and three light chain CDRs of SEQ ID Nos. 46, 47 and 48, respectively; or (ii) three heavy chain CDRs of SEQ ID Nos. 31, 32 and 33, respectively, and three light chain CDRs of SEQ ID Nos. 49, 50 and 51, respectively. In some embodiments, the antigen binding site that specifically binds CD28 is part of a polypeptide chain comprising the amino acid sequence of SEQ ID NO:3 or an amino acid sequence that is at least 95% identical to the amino acid sequence of SEQ ID NO:3 optionally comprising CDRs that are 100% identical to the CDRs of the polypeptide chain of SEQ ID NO:3. In some embodiments, the antigen binding site that specifically binds CD28 is part of a polypeptide chain comprising the amino acid sequence of SEQ ID NO:4 or an amino acid sequence that is at least 95% identical to the amino acid sequence of SEQ ID NO:4 optionally comprising CDRs that are 100% identical to the CDRs of the polypeptide chain of SEQ ID NO:4.

In some embodiments, a binding protein of the present disclosure comprises a first antigen binding site that specifically binds CD3, a second antigen binding site that specifically binds CD28, and a third antigen binding site that specifically binds CD38, or a first antigen binding site that specifically binds CD28, a second antigen binding site that specifically binds CD3, and a third antigen binding site that specifically binds CD38, wherein:
the antigen binding site specifically binding CD3 comprises (i) three heavy chain CDRs of SEQ ID Nos. 34, 35 and 36, respectively, and three light chain CDRs of SEQ ID Nos. 52, 53 and 54, respectively; or (ii) three heavy chain CDRs of SEQ ID Nos. 34, 35 and 36, respectively, and three light chain CDRs of SEQ ID Nos. 149, 53 and 54, respectively; and
the antigen binding site specifically binding CD28 comprises (i) three heavy chain CDRs of SEQ ID Nos. 28, 29 and 30, respectively, and three light chain CDRs of SEQ ID Nos. 46, 47 and 48, respectively; or (ii) three heavy chain CDRs of SEQ ID Nos. 31, 32 and 33, respectively, and three light chain CDRs of SEQ ID Nos. 49, 50 and 51, respectively; and
the antigen binding site specifically binding CD38 comprises (i) three heavy chain CDRs of SEQ ID Nos. 40, 41 and 42, respectively, and three light chain CDRs of SEQ ID Nos. 58, 44 and 59, respectively.

In some embodiments, the antigen binding site that specifically binds a tumor target protein comprises a heavy chain variable domain comprising the amino acid sequence of SEQ ID NO: 156 and a light chain variable domain comprising the amino acid sequence of SEQ ID NO: 157; a heavy chain variable domain comprising the amino acid sequence of SEQ ID NO: 158 and a light chain variable domain comprising the amino acid sequence of SEQ ID NO: 159; a heavy chain variable domain comprising the amino acid sequence of SEQ ID NO: 164 and a light chain variable domain comprising the amino acid sequence of SEQ ID NO: 165; a heavy chain variable domain comprising the amino acid sequence of SEQ ID NO: 150 and a light chain variable domain comprising the amino acid sequence of SEQ ID NO: 151; or a heavy chain variable domain comprising the amino acid sequence of SEQ ID NO: 166 and a light chain variable domain comprising the amino acid sequence of SEQ ID NO: 167. In some embodiments, the antigen binding site that specifically binds a tumor target protein comprises six CDRs, or a heavy chain and a light chain variable domain, shown in Tables 2-5. In some embodiments, the antigen binding site that specifically binds a tumor target protein comprises six CDRs of an anti-Her2, anti-CD19, anti-CD20, anti-CD38, or anti-LAMP1 binding domain shown in Tables 2-5.

In some embodiments, a binding protein of the present disclosure comprises four polypeptide chains that form three antigen binding sites, wherein a first polypeptide chain comprises a structure represented by the formula:

V_{L2}-L₁-V_{L1}-L₂-C_{L} [I]

and a second polypeptide chain comprises a structure represented by the formula:

V_{H1}-L₃-V_{H2}-L₄-C_{H1}-hinge-C_{H2}-C_{H3} [II]

and a third polypeptide chain comprises a structure represented by the formula:

V_{H3}-C_{H1} [III]

and a fourth polypeptide chain comprises a structure represented by the formula:

V_{L3}-C_{L} [IV]

wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain;
C_{H2} is an immunoglobulin C_{H2} heavy chain constant domain;
C_{H3} is an immunoglobulin C_{H3} heavy chain constant domain;
hinge is an immunoglobulin hinge region connecting the C_{H1} and C_{H2} domains; and
L₁, L₂, L₃ and L₄ are amino acid linkers;
wherein the polypeptide of formula I and the polypeptide of formula II form a cross-over light chain-heavy chain pair;
wherein:
V_{L1}, V_{L2} and V_{L3} each independently comprise a variable domain sequence as set forth in any one of SEQ ID NOs:169, 171, and 173; and
wherein:
V_{H1}, V_{H2}, and V_{H3} each independently comprise a variable domain sequence as set forth in any one of SEQ ID NOs:168, 170, and 172.

In some embodiments, a binding protein of the present disclosure comprises four polypeptide chains that form three antigen binding sites, wherein a first polypeptide chain comprises a structure represented by the formula:

V_{L2}-L₁-V_{L1}-L₂-C_{L} [I]

and a second polypeptide chain comprises a structure represented by the formula:

V_{H1}-L₃-V_{H2}-L₄-C_{H1}-hinge-C_{H2}-C_{H3} [II]

and a third polypeptide chain comprises a structure represented by the formula:

V_{H3}-C_{H1}-hinge-C_{H2}-C_{H3} [III]

and a fourth polypeptide chain comprises a structure represented by the formula:

V_{L3}-C_{L} [IV]

wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain;
C_{H2} is an immunoglobulin C_{H2} heavy chain constant domain;
C_{H3} is an immunoglobulin C_{H3} heavy chain constant domain;
hinge is an immunoglobulin hinge region connecting the C_{H1} and C_{H2} domains; and
L₁, L₂, L₃ and L₄ are amino acid linkers;
wherein the polypeptide of formula I and the polypeptide of formula II form a cross-over light chain-heavy chain pair;
wherein:
V_{L1}, V_{L2} and V_{L3} each independently comprise light chain complementarity determining regions comprising an amino acid sequence selected from the group consisting of SEQ ID NOs: 141-147, 178, and 179; and
wherein:
V_{H1}, V_{H2} and V_{H3} each independently comprise heavy chain complementarity determining regions comprising an amino acid sequence selected from the group consisting of SEQ ID NOs: 129-137.

In some embodiments, a binding protein of the present disclosure comprises four polypeptide chains that form three antigen binding sites, wherein a first polypeptide chain comprises a structure represented by the formula:

V_{L2}-L₁-V_{L1}-L₂-C_{L} [I]

and a second polypeptide chain comprises a structure represented by the formula:

V_{H1}-L₃-V_{H2}-L₄-C_{H1}-hinge-C_{H2}-C_{H3} [II]

and a third polypeptide chain comprises a structure represented by the formula:

V_{H3}-C_{H1}-hinge-C_{H2}-C_{H3} [III]

and a fourth polypeptide chain comprises a structure represented by the formula:

V_{L3}-C_{L} [IV]

wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain;
C_{H2} is an immunoglobulin C_{H2} heavy chain constant domain;
C_{H3} is an immunoglobulin C_{H3} heavy chain constant domain;
hinge is an immunoglobulin hinge region connecting the C_{H1} and C_{H2} domains; and
L₁, L₂, L₃ and L₄ are amino acid linkers;
wherein the polypeptide of formula I and the polypeptide of formula II form a cross-over light chain-heavy chain pair;
wherein:
V_{L1}, V_{L2} and V_{L3} each independently comprise a variable domain sequence as set forth in any one of SEQ ID NOs:151, 153, 155, 157, 159, 161, 163, 165, and 167; and
wherein:
V_{H1}, V_{H2}, and V_{H3} each independently comprise a variable domain sequence as set forth in any one of SEQ ID NOs:150, 152, 154, 156, 158, 160, 162, 164, and 166.

In some embodiments, a binding protein of the present disclosure comprises four polypeptide chains that form three antigen binding sites, wherein a first polypeptide chain comprises a structure represented by the formula:

V_{L2}-L₁-V_{L1}-L₂-C_{L} [I]

and a second polypeptide chain comprises a structure represented by the formula:

V_{H1}-L₃-V_{H2}-L₄-C_{H1}-hinge-C_{H2}-C_{H3} [II]

and a third polypeptide chain comprises a structure represented by the formula:

V_{H3}-C_{H1}-hinge-C_{H2}-C_{H3} [III]

and a fourth polypeptide chain comprises a structure represented by the formula:

V_{L3}-C_{L} [IV]

wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain;
C_{H2} is an immunoglobulin C_{H2} heavy chain constant domain;
C_{H3} is an immunoglobulin C_{H3} heavy chain constant domain;
hinge is an immunoglobulin hinge region connecting the C_{H1} and C_{H2} domains; and
L₁, L₂, L₃ and L₄ are amino acid linkers;
wherein the polypeptide of formula I and the polypeptide of formula II form a cross-over light chain-heavy chain pair;
wherein:
V_{L1}, V_{L2} and V_{L3} each independently comprise light chain complementarity determining regions comprising an amino acid sequence as set forth in any one of SEQ ID NOs: 43-59, 123-125, 138-140, and 149; and
wherein:
V_{H1}, V_{H2} and V_{H3} each independently comprise heavy chain complementarity determining regions comprising an amino acid sequence as set forth in any one of SEQ ID NOs: 25-42, 120-122, and 126-128.

In some embodiments, a binding protein of the present disclosure comprises an antigen binding site that specifically binds CD3, an antigen binding site that specifically binds CD28, and an antigen binding site that specifically binds an antigen target other than CD3 or CD28. In some embodiments, a binding protein of the present disclosure comprises an antigen binding site that specifically binds human CD3, an antigen binding site that specifically binds human CD28, and an antigen binding site that specifically binds a human antigen target other than CD3 or CD28. In some embodiments, a binding protein of the present disclosure comprises (a) an antigen binding site that specifically binds CD3, wherein the antigen binding site that specifically binds CD3 comprises (i) a heavy chain variable domain comprising the amino acid sequence of SEQ ID NO: 152 and a light chain variable domain comprising the amino acid sequence of SEQ ID NO: 153, (ii) a heavy chain variable domain comprising the amino acid sequence of SEQ ID NO: 154 and a light chain variable domain comprising the amino acid sequence of SEQ ID NO: 155, (iii) a heavy chain variable domain comprising a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36, and a light chain variable domain comprising a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54, or (iv) a heavy chain variable domain comprising a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36, and a light chain variable domain comprising a CDR-L1 comprising the amino acid sequence of SEQ ID NO:149, a CDR-L2 comprising the amino acid sequence of SEQ ID NO:53, and a CDR-L3 comprising the amino acid sequence of SEQ ID NO:54; (b) an antigen binding site that specifically binds CD28, wherein the antigen binding site that specifically binds CD28 comprises (i) a heavy chain variable domain comprising the amino acid sequence of SEQ ID NO: 160 and a light chain variable domain comprising the amino acid sequence of SEQ ID NO: 161, (ii) a heavy chain variable domain comprising the amino acid sequence of SEQ ID NO: 162 and a light chain variable domain comprising the amino acid sequence of SEQ ID NO: 163, (iii) a heavy chain variable domain comprising a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:30, and a light chain variable domain comprising a CDR-L1 comprising the amino acid sequence of SEQ ID NO:46, a CDR-L2 comprising the amino acid sequence of SEQ ID NO: 47, and a CDR-L3 comprising the amino acid sequence of SEQ ID NO:48, or (iv) a heavy chain variable domain comprising a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33, and a light chain variable domain comprising a CDR-L1 comprising the amino acid sequence of SEQ ID NO:49, a CDR-L2 comprising the amino acid sequence of SEQ ID NO:50, and a CDR-L3 comprising the amino acid sequence of SEQ ID NO:51; and (c) an antigen binding site that specifically binds an antigen target other than CD3 or CD28. In some embodiments, a binding protein of the present disclosure comprises a first polypeptide chain comprising the amino acid sequence of SEQ ID NO:4 or 10, a second polypeptide chain comprising the amino acid sequence of SEQ ID NO:3 or 9, and a third and a fourth polypeptide chain, wherein the third and fourth polypeptide chains form an antigen binding domain that specifically binds an antigen target other than CD3 or CD28. In some embodiments, the antigen binding site that specifically binds an antigen target other than CD3 or CD28 binds an antigen target selected from A2AR, APRIL, ATPDase, BAFF, BAFFR, BCMA, BlyS, BTK, BTLA, B7DC, B7H1, B7H4 (also known as VTCN1), B7H5, B7H6, B7H7, B7RP1, B7-4, C3, C5, CCL2 (also known as MCP-1), CCL3 (also known as MIP-1a), CCL4 (also known as MIP-1b), CCL5 (also known as RANTES), CCL7 (also known as MCP-3), CCL8 (also known as mcp-2), CCL11 (also known as eotaxin), CCL15 (also known as MIP-1d), CCL17 (also known as TARC), CCL19 (also known as MIP-3b), CCL20 (also known as MIP-3a), CCL21 (also known as MIP-2), CCL24 (also known as MPIF-2/eotaxin-2), CCL25 (also known as TECK), CCL26 (also known as eotaxin-3), CCR3, CCR4, CD19, CD20, CD23 (also known as FCER2, a receptor for IgE), CD24, CD27, CD38, CD39, CD40, CD70, CD80 (also known as B7-1), CD86 (also known as B7-2), CD122, CD137 (also known as 41BB), CD137L, CD152 (also known as CTLA4), CD154 (also known as CD40L), CD160, CD272, CD273 (also known as PDL2), CD274 (also known as PDL1), CD275 (also known as B7H2), CD276 (also known as B7H3), CD278 (also known as ICOS), CD279 (also known as PD-1), CDH1 (also known as E-cadherin), chitinase, CLEC9, CLEC91, CRTH2, CSF-1 (also known as M-CSF), CSF-2 (also known as GM-CSF), CSF-3 (also known as GCSF), CX3CL1 (also known as SCYD1), CXCL12 (also known as SDF1), CXCL13, CXCR3, DNGR-1, ectonucleoside triphosphate diphosphohydrolase 1, EGFR, ENTPD1, FCER1A, FCER1, FLAP, FOLH1, Gi24, GITR, GITRL, GM-CSF, Her2, HHLA2, HMGB1, HVEM, ICOSLG, IDO, IFNα, IgE, IGF1R, IL2Rbeta, IL1, IL1A, IL1B, IL1F10, IL2, IL4, IL4Ra, IL5, IL5R, IL6, IL7, IL7Ra, IL8, IL9, IL9R, IL10, rhIL10, IL12, IL13, IL13Ra1, IL13Ra2, IL15, IL17, IL17Rb (also known as a receptor for IL25), IL18, IL22, IL23, IL25, IL27, IL33, IL35, ITGB4 (also known as b4 integrin), ITK, KIR, LAG3, LAMP1, leptin, LPFS2, MHC class II, NCR3LG1, NKG2D, NTPDase-1, OX40, OX40L, PD-1H, platelet receptor, PROM1, S152, SISP1, SLC, SPG64, ST2 (also known as a receptor for IL33), STEAP2, Syk kinase, TACI, TDO, T14, TIGIT, TIM3, TLR, TLR2, TLR4, TLR5, TLR9, TMEF1, TNFa, TNFRSF7, Tp55, TREM1, TSLP (also known as a co-receptor for IL7Ra), TSLPR, TWEAK, VEGF, VISTA, Vstm3, WUCAM, and XCR1 (also known as GPR5/CCXCR1).

In some embodiments, V_{H1} comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:30; V_{L1} Comprises a CDR-L1 comprising the amino acid sequence of SEQ ID NO:46, a CDR-L2 comprising the amino acid sequence of SEQ ID NO: 47, and a CDR-L3 comprising the amino acid sequence of SEQ ID NO:48; V_{H2} comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} Comprises a CDR-H1 comprising the amino acid sequence of SEQ ID NO:25, a CDR-H2 comprising the amino acid sequence of SEQ ID NO:26, and a CDR-H3 comprising the amino acid sequence of SEQ ID NO:27; and V_{L3} comprises a CDR-L1 comprising the amino acid sequence of SEQ ID NO:43, a CDR-L2 comprising the amino acid sequence of SEQ ID NO:44, and a CDR-L3 comprising the amino acid sequence of SEQ ID NO:45.

In some embodiments, V_{H1} comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L1} Comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H2} comprises a CDR-H1 comprising the amino acid sequence of SEQ ID NO:25, a CDR-H2 comprising the amino acid sequence of SEQ ID NO:26, and a CDR-H3 comprising the amino acid sequence of SEQ ID NO:27; V_{L2} comprises a CDR-L1 comprising the amino acid sequence of SEQ ID NO:43, a CDR-L2 comprising the amino acid sequence of SEQ ID NO:44, and a CDR-L3 comprising the amino acid sequence of SEQ ID NO:45; V_{H3} Comprises a CDR-H1 comprising the amino acid sequence of SEQ ID NO:25, a CDR-H2 comprising the amino acid sequence of SEQ ID NO:26, and a CDR-H3 comprising the amino acid sequence of SEQ ID NO:27; and V_{L3} comprises a CDR-L1 comprising the amino acid sequence of SEQ ID NO:43, a CDR-L2 comprising the amino acid sequence of SEQ ID NO:44, and a CDR-L3 comprising the amino acid sequence of SEQ ID NO:45.

In some embodiments, V_{H1} comprises a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33; V_{L1} Comprises a CDR-L1 comprising the amino acid sequence of SEQ ID NO:49, a CDR-L2 comprising the amino acid sequence of SEQ ID NO:50, and a CDR-L3 comprising the amino acid sequence of SEQ ID NO:51; V_{H2} comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} Comprises a CDR-H1 comprising the amino acid sequence of SEQ ID NO:25, a CDR-H2 comprising the amino acid sequence of SEQ ID NO:26, and a CDR-H3 comprising the amino acid sequence of SEQ ID NO:27; and V_{L3} comprises a CDR-L1 comprising the amino acid sequence of SEQ ID NO:43, a CDR-L2 comprising the amino acid sequence of SEQ ID NO:44, and a CDR-L3 comprising the amino acid sequence of SEQ ID NO:45.

In some embodiments, V_{H1} comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:30; V_{L1} Comprises a CDR-L1 comprising the amino acid sequence of SEQ ID NO:46, a CDR-L2 comprising the amino acid sequence of SEQ ID NO: 47, and a CDR-L3 comprising the amino acid sequence of SEQ ID NO:48; V_{H2} comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} Comprises a CDR-H1 comprising the amino acid sequence of SEQ ID NO:37, a CDR-H2 comprising the amino acid sequence of SEQ ID NO:38, and a CDR-H3 comprising the amino acid sequence of SEQ ID NO:39; and V_{L3} comprises a CDR-L1 comprising the amino acid sequence of SEQ ID NO:55, a CDR-L2 comprising the amino acid sequence of SEQ ID NO:56, and a CDR-L3 comprising the amino acid sequence of SEQ ID NO:57.

In some embodiments, V_{H1} comprises a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33; V_{L1} Comprises a CDR-L1 comprising the amino acid sequence of SEQ ID NO:49, a CDR-L2 comprising the amino acid sequence of SEQ ID NO:50, and a CDR-L3 comprising the amino acid sequence of SEQ ID NO:51; V_{H2} comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} Comprises a CDR-H1 comprising the amino acid sequence of SEQ ID NO:37, a CDR-H2 comprising the amino acid sequence of SEQ ID NO:38, and a CDR-H3 comprising the amino acid sequence of SEQ ID NO:39; and V_{L3} comprises a CDR-L1 comprising the amino acid sequence of SEQ ID NO:55, a CDR-L2 comprising the amino acid sequence of SEQ ID NO:56, and a CDR-L3 comprising the amino acid sequence of SEQ ID NO:57.

In some embodiments, V_{H1} comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:30; V_{L1} Comprises a CDR-L1 comprising the amino acid sequence of SEQ ID NO:46, a CDR-L2 comprising the amino acid sequence of SEQ ID NO: 47, and a CDR-L3 comprising the amino acid sequence of SEQ ID NO:48; V_{H2} comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} Comprises a CDR-H1 comprising the amino acid sequence of SEQ ID NO:40, a CDR-H2 comprising the amino acid sequence of SEQ ID NO:41, and a CDR-H3 comprising the amino acid sequence of SEQ ID NO:42; and V_{L3} comprises a CDR-L1 comprising the amino acid sequence of SEQ ID NO:58, a CDR-L2 comprising the amino acid sequence of SEQ ID NO:44, and a CDR-L3 comprising the amino acid sequence of SEQ ID NO:59.

In some embodiments, V_{H1} comprises a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33; V_{L1} Comprises a CDR-L1 comprising the amino acid sequence of SEQ ID NO:49, a CDR-L2 comprising the amino acid sequence of SEQ ID NO:50, and a CDR-L3 comprising the amino acid sequence of SEQ ID NO:51; V_{H2} comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} Comprises a CDR-H1 comprising the amino acid sequence of SEQ ID NO:40, a CDR-H2 comprising the amino acid sequence of SEQ ID NO:41, and a CDR-H3 comprising the amino acid sequence of SEQ ID NO:42; and V_{L3} comprises a CDR-L1 comprising the amino acid sequence of SEQ ID NO:58, a CDR-L2 comprising the amino acid sequence of SEQ ID NO:44, and a CDR-L3 comprising the amino acid sequence of SEQ ID NO:59.

In some embodiments, V_{H1} comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:30; V_{L1} Comprises a CDR-L1 comprising the amino acid sequence of SEQ ID NO:46, a CDR-L2 comprising the amino acid sequence of SEQ ID NO: 47, and a CDR-L3 comprising the amino acid sequence of SEQ ID NO:48; V_{H2} comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} Comprises a CDR-H1 comprising the amino acid sequence of SEQ ID NO:126, a CDR-H2 comprising the amino acid sequence of SEQ ID NO:127, and a CDR-H3 comprising the amino acid sequence of SEQ ID NO:128; and V_{L3} comprises a CDR-L1 comprising the amino acid sequence of SEQ ID NO:138, a CDR-L2 comprising the amino acid sequence of SEQ ID NO:139, and a CDR-L3 comprising the amino acid sequence of SEQ ID NO:140.

In some embodiments, V_{H1} comprises a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33; V_{L1} Comprises a CDR-L1 comprising the amino acid sequence of SEQ ID NO:49, a CDR-L2 comprising the amino acid sequence of SEQ ID NO:50, and a CDR-L3 comprising the amino acid sequence of SEQ ID NO:51; V_{H2} comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} Comprises a CDR-H1 comprising the amino acid sequence of SEQ ID NO:126, a CDR-H2 comprising the amino acid sequence of SEQ ID NO:127, and a CDR-H3 comprising the amino acid sequence of SEQ ID NO:128; and V_{L3} comprises a CDR-L1 comprising the amino acid sequence of SEQ ID NO:138, a CDR-L2 comprising the amino acid sequence of SEQ ID NO:139, and a CDR-L3 comprising the amino acid sequence of SEQ ID NO:140.

In some embodiments, V_{H1} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; V_{L1} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144; V_{H2} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; V_{L2} Comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; V_{H3} comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131; and V_{L3} comprises a CDR-L1 comprising the amino acid sequence of SEQ ID NO:141, a CDR-L2 comprising the amino acid sequence of SEQ ID NO:178, and a CDR-L3 comprising the amino acid sequence of SEQ ID NO:142.

In some embodiments, V_{H1} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; V_{L1} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; V_{H2} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; V_{L2} Comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144; V_{H3} comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131; and V_{L3} comprises a CDR-L1 comprising the amino acid sequence of SEQ ID NO:141, a CDR-L2 comprising the amino acid sequence of SEQ ID NO:178, and a CDR-L3 comprising the amino acid sequence of SEQ ID NO:142.

In some embodiments, V_{H1} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; V_{L1} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; V_{H2} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; V_{L2} Comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144; V_{H3} comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131; and V_{L3} comprises a CDR-L1 comprising the amino acid sequence of SEQ ID NO:141, a CDR-L2 comprising the amino acid sequence of SEQ ID NO:178, and a CDR-L3 comprising the amino acid sequence of SEQ ID NO:142.

In some embodiments, V_{H1} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; V_{L1} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144; V_{H2} comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131; V_{L2} Comprises a CDR-L1 comprising the amino acid sequence of SEQ ID NO:141, a CDR-L2 comprising the amino acid sequence of SEQ ID NO:178, and a CDR-L3 comprising the amino acid sequence of SEQ ID NO:142; V_{H3} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; and V_{L3} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147.

In some embodiments, V_{H1} comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131; V_{L1} comprises a CDR-L1 comprising the amino acid sequence of SEQ ID NO:141, a CDR-L2 comprising the amino acid sequence of SEQ ID NO:178, and a CDR-L3 comprising the amino acid sequence of SEQ ID NO:142; V_{H2} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; V_{L2} Comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144; V_{H3} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; and V_{L3} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147.

In some embodiments, V_{H1} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; V_{L1} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; V_{H2} comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131; V_{L2} Comprises a CDR-L1 comprising the amino acid sequence of SEQ ID NO:141, a CDR-L2 comprising the amino acid sequence of SEQ ID NO:178, and a CDR-L3 comprising the amino acid sequence of SEQ ID NO:142; V_{H3} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; and V_{L3} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144.

In some embodiments, V_{H1} comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131; V_{L1} comprises a CDR-L1 comprising the amino acid sequence of SEQ ID NO:141, a CDR-L2 comprising the amino acid sequence of SEQ ID NO:178, and a CDR-L3 comprising the amino acid sequence of SEQ ID NO:142; V_{H2} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; V_{L2} Comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; V_{H3} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; and V_{L3} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144.

In some embodiments, V_{H1} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; V_{L1} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; V_{H2} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; V_{L2} Comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144; V_{H3} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; and V_{L3} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147.

In some embodiments, V_{H1} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; V_{L1} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; V_{H2} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; V_{L2} Comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144; V_{H3} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; and V_{L3} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144.

In some embodiments, V_{H1} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; V_{L1} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144; V_{H2} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; V_{L2} Comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; V_{H3} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; and V_{L3} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147.

In some embodiments, V_{H1} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; V_{L1} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144; V_{H2} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; V_{L2} Comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; V_{H3} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; and V_{L3} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144.

In some embodiments, V_{H1} comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:30; V_{L1} Comprises a CDR-L1 comprising the amino acid sequence of SEQ ID NO:46, a CDR-L2 comprising the amino acid sequence of SEQ ID NO: 47, and a CDR-L3 comprising the amino acid sequence of SEQ ID NO:48; V_{H2} comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} Comprises a CDR-H1 comprising the amino acid sequence of SEQ ID NO:120, a CDR-H2 comprising the amino acid sequence of SEQ ID NO:121, and a CDR-H3 comprising the amino acid sequence of SEQ ID NO:122; and V_{L3} comprises a CDR-L1 comprising the amino acid sequence of SEQ ID NO:123, a CDR-L2 comprising the amino acid sequence of SEQ ID NO:124, and a CDR-L3 comprising the amino acid sequence of SEQ ID NO:125.

In some embodiments, V_{H1} comprises a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33; V_{L1} Comprises a CDR-L1 comprising the amino acid sequence of SEQ ID NO:49, a CDR-L2 comprising the amino acid sequence of SEQ ID NO:50, and a CDR-L3 comprising the amino acid sequence of SEQ ID NO:51; V_{H2} comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} comprises a CDR-H1 comprising the amino acid sequence of SEQ ID NO:120, a CDR-H2 comprising the amino acid sequence of SEQ ID NO:121, and a CDR-H3 comprising the amino acid sequence of SEQ ID NO:122; and V_{L3} comprises a CDR-L1 comprising the amino acid sequence of SEQ ID NO:123, a CDR-L2 comprising the amino acid sequence of SEQ ID NO:124, and a CDR-L3 comprising the amino acid sequence of SEQ ID NO:125.

### Antigen Targets

In some embodiments, a binding protein of the present disclosure binds one or more (e.g., one, two, or three) of the following antigen targets or target proteins: A2AR, APRIL, ATPDase, BAFF, BAFFR, BCMA, BlyS, BTK, BTLA, B7DC, B7H1, B7H4 (also known as VTCN1), B7H5, B7H6, B7H7, B7RP1, B7-4, C3, C5, CCL2 (also known as MCP-1), CCL3 (also known as MIP-1a), CCL4 (also known as MIP-1b), CCL5 (also known as RANTES), CCL7 (also known as MCP-3), CCL8 (also known as mcp-2), CCL11 (also known as eotaxin), CCL15 (also known as MIP-1d), CCL17 (also known as TARC), CCL19 (also known as MIP-3b), CCL20 (also known as MIP-3a), CCL21 (also known as MIP-2), CCL24 (also known as MPIF-2/eotaxin-2), CCL25 (also known as TECK), CCL26 (also known as eotaxin-3), CCR3, CCR4, CD3, CD19, CD20, CD23 (also known as FCER2, a receptor for IgE), CD24, CD27, CD28, CD38, CD39, CD40, CD70, CD80 (also known as B7-1), CD86 (also known as B7-2), CD122, CD137 (also known as 41BB), CD137L, CD152 (also known as CTLA4), CD154 (also known as CD40L), CD160, CD272, CD273 (also known as PDL2), CD274 (also known as PDL1), CD275 (also known as B7H2), CD276 (also known as B7H3), CD278 (also known as ICOS), CD279 (also known as PD-1), CDH1 (also known as E-cadherin), chitinase, CLEC9, CLEC91, CRTH2, CSF-1 (also known as M-CSF), CSF-2 (also known as GM-CSF), CSF-3 (also known as GCSF), CX3CL1 (also known as SCYD1), CXCL12 (also known as SDF1), CXCL13, CXCR3, DNGR-1, ectonucleoside triphosphate diphosphohydrolase 1, EGFR, ENTPD1, FCER1A, FCER1, FLAP, FOLH1, Gi24, GITR, GITRL, GM-CSF, Her2, HHLA2, HMGB1, HVEM, ICOSLG, IDO, IFNα, IgE, IGF1R, IL2Rbeta, IL1, IL1A, IL1B, IL1F10, IL2, IL4, IL4Ra, IL5, IL5R, IL6, IL7, IL7Ra, IL8, IL9, IL9R, IL10, rhIL10, IL12, IL13, IL13Ra1, IL13Ra2, IL15, IL17, IL17Rb (also known as a receptor for IL25), IL18, IL22, IL23, IL25, IL27, IL33, IL35, ITGB4 (also known as b4 integrin), ITK, KIR, LAG3, LAMP1, leptin, LPFS2, MHC class II, NCR3LG1, NKG2D, NTPDase-1, OX40, OX40L, PD-1H, platelet receptor, PROM1, S152, SISP1, SLC, SPG64, ST2 (also known as a receptor for IL33), STEAP2, Syk kinase, TACI, TDO, T14, TIGIT, TIM3, TLR, TLR2, TLR4, TLR5, TLR9, TMEF1, TNFa, TNFRSF7, Tp55, TREM1, TSLP (also known as a co-receptor for IL7Ra), TSLPR, TWEAK, VEGF, VISTA, Vstm3, WUCAM, and XCR1 (also known as GPR5/CCXCR1). In some embodiments, one or more of the above antigen targets are human antigen targets.

In one embodiment, the binding proteins specifically bind to one or more tumor antigen targets (e.g., target proteins). In other embodiments, the binding proteins specifically bind to one or more tumor target protein and one or more target protein on a T-cell including a T cell receptor complex. These T-cell engager binding proteins are capable of recruiting T cells transiently to target cells and, at the same time, activating the cytolytic activity of the T cells. Examples of target proteins on T cells include but are not limited to CD3 and CD28, among others. Further examples of such antigen targets or target proteins are provided supra. In some embodiments, the trispecific binding proteins may be generated by combining the antigen binding domains of two or more monospecific antibodies (parent antibodies) into one antibody. In some embodiments, a binding protein of the present disclosure binds one or more (e.g., one, two, or three) of the following antigen targets: CD3, CD19, CD20, CD28, CD38, Her2, LAMP1, IL-4, IL-13 and TNFa.

In some embodiments of the disclosure, the trivalent binding protein is capable of binding three antigen targets. In some embodiments of the disclosure, the trivalent binding protein is capable of binding three different antigen targets. In one embodiment, the binding protein is trispecific and one light chain-heavy chain pair is capable of binding two different antigen targets or epitopes and one light chain-heavy chain pair is capable of binding one antigen target or epitope. In another embodiment, the binding protein is capable of binding three tumor antigen targets. In another embodiment, the binding protein is capable of binding three different tumor antigen targets. In other embodiments, the binding protein is capable of inhibiting the function of one or more of the antigen targets.

In some embodiments, a binding protein of the present disclosure binds one or more tumor target proteins. In some embodiments, the binding protein is capable of specifically binding three epitopes on a single tumor target protein. In some embodiments, the binding protein is capable of specifically binding three different epitopes on a single tumor target protein. In some embodiments, the binding protein is capable of binding two different epitopes on a first tumor target protein, and one epitope on a second tumor target protein. In some embodiments, the first and second tumor target proteins are different. In some embodiments, the binding protein is capable of specifically binding three different tumor target proteins.

In some embodiments, a binding protein of the present disclosure binds one or more cytokine target proteins. In some embodiments, the binding protein is capable of specifically binding three epitopes on a single cytokine target protein. In some embodiments, the binding protein is capable of specifically binding three different epitopes on a single cytokine target protein. In some embodiments, the binding protein is capable of binding two different epitopes on a first cytokine target protein, and one epitope on a second cytokine target protein. In some embodiments, the first and second cytokine target proteins are different. In some embodiments, the binding protein is capable of specifically binding three different cytokine target proteins. In some embodiments, the one or more cytokine target proteins are one or more of IL-4, IL-13 and/or TNFa. Further examples of cytokine target proteins are provided infra.

In some embodiments, a binding protein of the present disclosure binds one or more tumor target proteins and one or more T cell target proteins. In some embodiments, the binding protein is capable of specifically binding one tumor target protein and two different epitopes on a single T cell target protein. In some embodiments, the binding protein is capable of specifically binding one tumor target protein and two different T cell target proteins (e.g., CD28 and CD3). In some embodiments, the binding protein is capable of specifically binding one T cell target protein and two different epitopes on a single tumor target protein. In some embodiments, the binding protein is capable of specifically binding one T cell target protein and two different tumor target proteins. In some embodiments, the first and second polypeptide chains of the binding protein form two antigen binding sites that specifically target two T cell target proteins, and the third and fourth polypeptide chains of the binding protein form an antigen binding site that specifically binds a tumor target protein. In some embodiments, the first and second polypeptide chains of the binding protein form two antigen binding sites that specifically target two tumor target proteins, and the third and fourth polypeptide chains of the binding protein form an antigen binding site that specifically binds a T cell target protein. In some embodiments, the one or more tumor target proteins are one or more of CD3, CD19, CD20, CD28, CD38, Her2, LAMP1, IL-4, IL-13 and/or TNFa. In some embodiments, the one or more T cell target proteins are one or more of CD3 and CD28. Further examples of tumor target proteins and T cell target proteins are provided supra.

In some embodiments, a binding protein of the present disclosure binds, independently of each other, same or different, one, two or three antigen targets or target proteins, selected from cytokine target proteins, tumor target antigens or tumor target proteins, T cell target proteins, immune checkpoint inhibitors, immune checkpoint modulators, immune checkpoint costimulatory molecules, and/or target molecules on the surface of an immune cell. In some embodiments, a binding protein of the present disclosure is trivalent but bispecific and capable of specifically binding twice to the same antigen targets or target proteins. In some embodiments, a binding protein of the present disclosure is capable of specifically binding two different epitopes on a single cytokine target proteins, tumor target antigens or tumor target proteins, T cell target proteins, immune checkpoint inhibitors, immune checkpoint modulators, immune checkpoint costimulatory molecules, and/or target molecules on the surface of an immune cell. Further examples of such antigen targets or target proteins are provided supra.

The binding proteins of the disclosure may be prepared using domains or sequences obtained or derived from any human or non-human antibody, including, for example, human, murine, or humanized antibodies.

### Linkers

In some embodiments, the linkers L₁, L₂, L₃ and L₄ range from no amino acids (length=0) to about 100 amino acids long, or less than 100, 50, 40, 30, 20, or 15 amino acids or less. The linkers can also be 10, 9, 8, 7, 6, 5, 4, 3, 2, or 1 amino acids long. L₁, L₂, L₃ and L₄ in one binding protein may all have the same amino acid sequence or may all have different amino acid sequences.

Examples of suitable linkers include a single glycine (Gly) residue; a diglycine peptide (Gly-Gly); a tripeptide (Gly-Gly-Gly); a peptide with four glycine residues (Gly-Gly-Gly-Gly; SEQ ID NO: 98); a peptide with five glycine residues (Gly-Gly-Gly-Gly-Gly; SEQ ID NO: 99); a peptide with six glycine residues (Gly-Gly-Gly-Gly-Gly-Gly; SEQ ID NO: 100); a peptide with seven glycine residues (Gly-Gly-Gly-Gly-Gly-Gly-Gly; SEQ ID NO: 101); a peptide with eight glycine residues (Gly-Gly-Gly-Gly-Gly-Gly-Gly-Gly; SEQ ID NO: 102). Other combinations of amino acid residues may be used such as the peptide Gly-Gly-Gly-Gly-Ser (SEQ ID NO: 103), the peptide Gly-Gly-Gly-Gly-Ser-Gly-Gly-Gly-Gly-Ser (SEQ ID NO: 104), the peptide Gly-Gly-Gly-Gly-Ser-Gly-Gly-Gly-Gly-Ser-Gly-Gly-Gly-Gly-Ser (SEQ ID NO: 105), and the peptide Gly-Gly-Ser-Gly-Ser-Ser-Gly-Ser-Gly-Gly (SEQ ID NO:148). Other suitable linkers include a single Ser, and Val residue; the dipeptide Arg-Thr, Gln-Pro, Ser-Ser, Thr-Lys, and Ser-Leu; Thr-Lys-Gly-Pro-Ser (SEQ ID NO: 106), Thr-Val-Ala-Ala-Pro (SEQ ID NO: 107), Gln-Pro-Lys-Ala-Ala (SEQ ID NO: 108), Gln-Arg-Ile-Glu-Gly (SEQ ID NO: 109); Ala-Ser-Thr-Lys-Gly-Pro-Ser (SEQ ID NO: 110), Arg-Thr-Val-Ala-Ala-Pro-Ser (SEQ ID NO:111), Gly-Gln-Pro-Lys-Ala-Ala-Pro (SEQ ID NO:112), and His-Ile-Asp-Ser-Pro-Asn-Lys (SEQ ID NO:113). The examples listed above are not intended to limit the scope of the disclosure in any way, and linkers comprising randomly selected amino acids selected from the group consisting of valine, leucine, isoleucine, serine, threonine, lysine, arginine, histidine, aspartate, glutamate, asparagine, glutamine, glycine, and proline have been shown to be suitable in the binding proteins. For additional descriptions of linker sequences, see, e.g., WO2012135345.

The identity and sequence of amino acid residues in the linker may vary depending on the type of secondary structural element necessary to achieve in the linker. For example, glycine, serine, and alanine are best for linkers having maximum flexibility. Some combination of glycine, proline, threonine, and serine are useful if a more rigid and extended linker is necessary. Any amino acid residue may be considered as a linker in combination with other amino acid residues to construct larger peptide linkers as necessary depending on the desired properties.

In some embodiments, the length of L₁ is at least twice the length of L₃. In some embodiments, the length of L₂ is at least twice the length of L₄. In some embodiments, the length of L₁ is at least twice the length of L₃, and the length of L₂ is at least twice the length of L₄. In some embodiments, L₁ is 3 to 12 amino acid residues in length, L₂ is 3 to 14 amino acid residues in length, L₃ is 1 to 8 amino acid residues in length, and L₄ is 1 to 3 amino acid residues in length. In some embodiments, L₁ is 5 to 10 amino acid residues in length, L₂ is 5 to 8 amino acid residues in length, L₃ is 1 to 5 amino acid residues in length, and L₄ is 1 to 2 amino acid residues in length. In some embodiments, L₁ is 7 amino acid residues in length, L₂ is 5 amino acid residues in length, L₃ is 1 amino acid residue in length, and L₄ is 2 amino acid residues in length. In some embodiments, L₁ is 10 amino acid residues in length, L₂ is 10 amino acid residues in length, L₃ is 0 amino acid residue in length, and L₄ is 0 amino acid residues in length. In some embodiments, L₁, L₂, L₃, and L₄ each have an independently selected length from 0 to 15 amino acids (e.g., 0, 1, 2, 3, 4, 5, 6, 7, 8, 9, 10, 11, 12, 13, 14, or 15 amino acids), wherein at least two of the linkers have a length of 1 to 15 amino acids (e.g., 1, 2, 3, 4, 5, 6, 7, 8, 9, 10, 11, 12, 13, 14, or 15 amino acids). In some embodiments, L₁, L₂, L₃, and L₄ are each 0 amino acids in length.

In some embodiments, L₁, L₂, L₃, and/or L₄ comprise the sequence Asp-Lys-Thr-His-Thr (SEQ ID NO: 525). In some embodiments, L₁ comprises the sequence Asp-Lys-Thr-His-Thr (SEQ ID NO: 525). In some embodiments, L₃ comprises the sequence Asp-Lys-Thr-His-Thr (SEQ ID NO: 525).

In some embodiments, L₁, L₂, L₃, and/or L₄ comprise a sequence derived from a naturally occurring sequence at the junction between an antibody variable domain and an antibody constant domain (e.g., as described in WO2012/135345). For example, in some embodiments, the linker comprises a sequence found at the transition between an endogenous V_{H} and C_{H1} domain, or between an endogenous V_{L} and C_{L} domain (e.g., kappa or lambda). In some embodiments, the linker comprises a sequence found at the transition between an endogenous human V_{H} and C_{H1} domain, or between an endogenous human V_{L} and C_{L} domain (e.g., human kappa or lambda).

In some embodiments, L₁, L₂, L₃, and/or L₄ comprise the sequence Gly-Gln-Pro-Lys-Ala-Ala-Pro (SEQ ID NO: 175). In some embodiments, L₁ comprises the sequence Gly-Gln-Pro-Lys-Ala-Ala-Pro (SEQ ID NO: 175). In some embodiments, L₁ comprises the sequence Gly-Gln-Pro-Lys-Ala-Ala-Pro (SEQ ID NO: 175), L₂ comprises the sequence Thr-Lys-Gly-Pro-Ser-Arg (SEQ ID NO: 176), L₃ comprises the sequence Ser, and L₄ comprises the sequence Arg-Thr. In some embodiments, L₃ comprises the sequence Gly-Gln-Pro-Lys-Ala-Ala-Pro (SEQ ID NO: 175). In some embodiments, L₁ comprises the sequence Ser, L₂ comprises the sequence Arg-Thr, L₃ comprises the sequence Gly-Gln-Pro-Lys-Ala-Ala-Pro (SEQ ID NO: 175) and L₄ comprises the sequence Thr-Lys-Gly-Pro-Ser-Arg (SEQ ID NO: 176).

In some embodiments, L₁, L₂, L₃ and L₄ each independently comprise a sequence selected from (GGGGS)ₙ (wherein n is an integer between 0 and 5; SEQ ID NO:174), GGGGSGGGGS (SEQ ID NO:104), GGGGSGGGGSGGGGS (SEQ ID NO:105), S, RT, TKGPS (SEQ ID NO:106), GQPKAAP (SEQ ID NO: 175), and GGSGSSGSGG (SEQ ID NO:148). In some embodiments, L₁ comprises the sequence GQPKAAP (SEQ ID NO: 175), L₂ comprises the sequence TKGPS (SEQ ID NO:106), L₃ comprises the sequence S, and L₄ comprises the sequence RT. In some embodiments, L₁ comprises the sequence GGGGSGGGGS (SEQ ID NO:104), L₂ comprises the sequence GGGGSGGGGS (SEQ ID NO:104), L₃ is 0 amino acids in length, and L₄ is 0 amino acids in length. In some embodiments, L₁ comprises the sequence GGSGSSGSGG (SEQ ID NO:148), L₂ comprises the sequence GGSGSSGSGG (SEQ ID NO:148), L₃ is 0 amino acids in length, and L₄ is 0 amino acids in length. In some embodiments, L₁ comprises the sequence GGGGSGGGGSGGGGS (SEQ ID NO:105), L₂ is 0 amino acids in length, L₃ comprises the sequence GGGGSGGGGSGGGGS (SEQ ID NO:105), and L₄ is 0 amino acids in length. In some embodiments, L₁ and L₂ are zero amino acids in length, and L₃ and L₄ each comprise an independently selected sequence selected from (GGGGS)ₙ (wherein n is an integer between 0 and 5; SEQ ID NO:174), GGGGSGGGGS (SEQ ID NO:104), GGGGSGGGGSGGGGS (SEQ ID NO:105), S, RT, TKGPS (SEQ ID NO:106), GQPKAAP (SEQ ID NO: 175), and GGSGSSGSGG (SEQ ID NO:148). In some embodiments, L₃ and L₄ are zero amino acids in length, and L₁ and L₂ each comprise an independently selected sequence selected from (GGGGS)ₙ (wherein n is an integer between 0 and 5; SEQ ID NO:174), GGGGSGGGGS (SEQ ID NO:104), GGGGSGGGGSGGGGS (SEQ ID NO:105), S, RT, TKGPS (SEQ ID NO:106), GQPKAAP (SEQ ID NO: 175), and GGSGSSGSGG (SEQ ID NO:148).

### Fc Regions and Constant Domains

In some embodiments, a binding protein of the present disclosure comprises a second polypeptide chain further comprising an Fc region linked to CH1, the Fc region comprising an immunoglobulin hinge region and CH2 and CH3 immunoglobulin heavy chain constant domains. In some embodiments, a binding protein of the present disclosure comprises a third polypeptide chain further comprising an Fc region linked to CH1, the Fc region comprising an immunoglobulin hinge region and CH2 and CH3 immunoglobulin heavy chain constant domains. In some embodiments, a binding protein of the present disclosure comprises a second polypeptide chain further comprising an Fc region linked to CH1, the Fc region comprising an immunoglobulin hinge region and CH2 and CH3 immunoglobulin heavy chain constant domains, and a third polypeptide chain further comprising an Fc region linked to CH1, the Fc region comprising an immunoglobulin hinge region and CH2 and CH3 immunoglobulin heavy chain constant domains.

In some embodiments, a binding protein of the present disclosure includes one or two Fc variants. The term “Fc variant” as used herein refers to a molecule or sequence that is modified from a native Fc but still comprises a binding site for the salvage receptor, FcRn (neonatal Fc receptor). Exemplary Fc variants, and their interaction with the salvage receptor, are known in the art. Thus, the term “Fc variant” can comprise a molecule or sequence that is humanized from a non-human native Fc. Furthermore, a native Fc comprises regions that can be removed because they provide structural features or biological activity that are not required for the antibody-like binding proteins of the invention. Thus, the term “Fc variant” comprises a molecule or sequence that lacks one or more native Fc sites or residues, or in which one or more Fc sites or residues has be modified, that affect or are involved in: (1) disulfide bond formation, (2) incompatibility with a selected host cell, (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

To improve the yields of the binding proteins, the C_{H3} domains can be altered by the “knob-into-holes” technology which is described in detail with several examples in, for example, International Publication No. WO 96/027011, Ridgway et al., 1996, Protein Eng. 9: 617-21; and Merchant et al., 1998, Nat. Biotechnol. 16: 677-81. Specifically, the interaction surfaces of the two C_{H3} domains are altered to increase the heterodimerisation of both heavy chains containing these two C_{H3} domains. Each of the two C_{H3} domains (of the two heavy chains) can be the “knob,” while the other is the “hole.” The introduction of a disulfide bridge further stabilizes the heterodimers (Merchant et al., 1998; Atwell et al., 1997, J. Mol. Biol. 270: 26-35) and increases the yield. In particular embodiments, the knob is on the second pair of polypeptides with a single variable domain. In other embodiments, the knob is on the first pair of polypeptides having the cross-over orientation. In yet other embodiments, the C_{H3} domains do not include a knob in hole.

In some embodiments, a binding protein of the present disclosure comprises a “knob” mutation on the second polypeptide chain and a “hole” mutation on the third polypeptide chain. In some embodiments, a binding protein of the present disclosure comprises a “knob” mutation on the third polypeptide chain and a “hole” mutation on the second polypeptide chain. In some embodiments, the “knob” mutation comprises substitution(s) at positions corresponding to positions 354 and/or 366 of human IgG1 or IgG4 according to EU Index. In some embodiments, the amino acid substitutions are S354C, T366W, T366Y, S354C and T366W, or S354C and T366Y. In some embodiments, the “knob” mutation comprises substitutions at positions corresponding to positions 354 and 366 of human IgG1 or IgG4 according to EU Index. In some embodiments, the amino acid substitutions are S354C and T366W. In some embodiments, the “hole” mutation comprises substitution(s) at positions corresponding to positions 407 and, optionally, 349, 366, and/or 368 and of human IgG1 or IgG4 according to EU Index. In some embodiments, the amino acid substitutions are Y407V or Y407T and optionally Y349C, T366S, and/or L368A. In some embodiments, the “hole” mutation comprises substitutions at positions corresponding to positions 349, 366, 368, and 407 of human IgG1 or IgG4 according to EU Index. In some embodiments, the amino acid substitutions are Y349C, T366S, L368A, and Y407V.

In some embodiments, the second polypeptide chain further comprises a first Fc region linked to CH1, the first Fc region comprising an immunoglobulin hinge region and CH2 and CH3 immunoglobulin heavy chain constant domains, wherein the first Fc region comprises amino acid substitution(s) at positions corresponding to positions 366 and optionally 354 of human IgG1 or IgG4 according to EU Index, wherein the amino acid substitutions are T366W or T366Y and optionally S354C; and wherein the third polypeptide chain further comprises a second Fc region linked to CH1, the second Fc region comprising an immunoglobulin hinge region and CH2 and CH3 immunoglobulin heavy chain constant domains, wherein the second Fc region comprises amino acid substitution(s) at positions corresponding to positions 407 and optionally 349, 366, and/or 368 and of human IgG1 or IgG4 according to EU Index, wherein the amino acid substitutions are Y407V or Y407T and optionally Y349C, T366S, and/or L368A.

In some embodiments, the second polypeptide chain further comprises a first Fc region linked to CH1, the first Fc region comprising an immunoglobulin hinge region and CH2 and CH3 immunoglobulin heavy chain constant domains, wherein the first Fc region comprises amino acid substitution(s) at positions corresponding to positions 407 and optionally 349, 366, and/or 368 and of human IgG1 or IgG4 according to EU Index, wherein the amino acid substitutions are Y407V or Y407T and optionally Y349C, T366S, and/or L368A; and wherein the third polypeptide chain further comprises a second Fc region linked to CH1, the second Fc region comprising an immunoglobulin hinge region and CH2 and CH3 immunoglobulin heavy chain constant domains, wherein the second Fc region comprises amino acid substitution(s) at positions corresponding to positions 366 and optionally 354 of human IgG1 or IgG4 according to EU Index, wherein the amino acid substitutions are T366W or T366Y and optionally S354C.

In some embodiments, the second polypeptide chain further comprises a first Fc region linked to CH1, the first Fc region comprising an immunoglobulin hinge region and CH2 and CH3 immunoglobulin heavy chain constant domains, wherein the first Fc region comprises amino acid substitution at position corresponding to position 366 of human IgG1 or IgG4 according to EU Index, wherein the amino acid substitution is T366W; and wherein the third polypeptide chain further comprises a second Fc region linked to CH1, the second Fc region comprising an immunoglobulin hinge region and CH2 and CH3 immunoglobulin heavy chain constant domains, wherein the second Fc region comprises amino acid substitution(s) at positions corresponding to positions 366, 368, and/or 407 and of human IgG1 or IgG4 according to EU Index, wherein the amino acid substitutions are T366S, L368A, and/or Y407V.

In some embodiments, the second polypeptide chain further comprises a first Fc region linked to CH1, the first Fc region comprising an immunoglobulin hinge region and CH2 and CH3 immunoglobulin heavy chain constant domains, wherein the first Fc region comprises amino acid substitution(s) at positions corresponding to positions 366, 368, and/or 407 and of human IgG1 or IgG4 according to EU Index, wherein the amino acid substitutions are T366S, L368A, and/or Y407V; and wherein the third polypeptide chain further comprises a second Fc region linked to CH1, the second Fc region comprising an immunoglobulin hinge region and CH2 and CH3 immunoglobulin heavy chain constant domains, wherein the second Fc region comprises amino acid substitution at position corresponding to position 366 of human IgG1 or IgG4 according to EU Index, wherein the amino acid substitution is T366W.

In some embodiments, the second polypeptide chain further comprises a first Fc region linked to CH1, the first Fc region comprising an immunoglobulin hinge region and CH2 and CH3 immunoglobulin heavy chain constant domains, wherein the first Fc region comprises amino acid substitutions at positions corresponding to positions 354 and 366 of human IgG1 or IgG4 according to EU Index, wherein the amino acid substitutions are S354C and T366W; and wherein the third polypeptide chain further comprises a second Fc region linked to CH1, the second Fc region comprising an immunoglobulin hinge region and CH2 and CH3 immunoglobulin heavy chain constant domains, wherein the second Fc region comprises amino acid substitutions at positions corresponding to positions 349, 366, 368, and 407 of human IgG1 or IgG4 according to EU Index, wherein the amino acid substitutions are Y349C, T366S, L368A, and Y407V. In some embodiments, the second polypeptide chain further comprises a first Fc region linked to CH1, the first Fc region comprising an immunoglobulin hinge region and CH2 and CH3 immunoglobulin heavy chain constant domains, wherein the first Fc region comprises amino acid substitutions at positions corresponding to positions 349, 366, 368, and 407 of human IgG1 or IgG4 according to EU Index, wherein the amino acid substitutions are Y349C, T366S, L368A, and Y407V; and wherein the third polypeptide chain further comprises a second Fc region linked to CH1, the second Fc region comprising an immunoglobulin hinge region and CH2 and CH3 immunoglobulin heavy chain constant domains, wherein the second Fc region comprises amino acid substitutions at positions corresponding to positions 354 and 366 of human IgG1 or IgG4 according to EU Index, wherein the amino acid substitutions are S354C and T366W. In some embodiments, the first and/or second Fc regions are human IgG1 Fc regions. In some embodiments, the first and/or second Fc regions are human IgG4 Fc regions.

In some embodiments, a binding protein of the present disclosure comprises one or more mutations to improve serum half-life (See e.g., Hinton, P. R. et al. (2006) J. Immunol. 176(1):346-56). In some embodiments, the mutation comprises substitutions at positions corresponding to positions 428 and 434 of human IgG1 or IgG4 according to EU Index, wherein the amino acid substitutions are M428L and N434S. In some embodiments, the binding protein comprises a second polypeptide chain further comprising a first Fc region linked to CH1, the first Fc region comprising an immunoglobulin hinge region and CH2 and CH3 immunoglobulin heavy chain constant domains, and a third polypeptide chain further comprising a second Fc region linked to CH1, the second Fc region comprising an immunoglobulin hinge region and CH2 and CH3 immunoglobulin heavy chain constant domains, wherein the first and/or second Fc regions comprise amino acid substitutions at positions corresponding to positions 428 and 434 of human IgG1 or IgG4 according to EU Index, wherein the amino acid substitutions are M428L and N434S. In some embodiments, a binding protein of the present disclosure comprises knob and hole mutations and one or more mutations to improve serum half-life. In some embodiments, the first and/or second Fc regions are human IgG1 Fc regions. In some embodiments, the first and/or second Fc regions are human IgG4 Fc regions.

In some embodiments, a binding protein of the present disclosure comprises one or more mutations to improve stability, e.g., of the hinge region and/or dimer interface of IgG4 (See e.g., Spiess, C. et al. (2013) J. Biol. Chem. 288:26583-26593). In some embodiments, the mutation comprises substitutions at positions corresponding to positions 228 and 409 of human IgG4 according to EU Index, wherein the amino acid substitutions are S228P and R409K. In some embodiments, the binding protein comprises a second polypeptide chain further comprising a first Fc region linked to C_{H1}, the first Fc region comprising an immunoglobulin hinge region and C_{H2} and C_{H3} immunoglobulin heavy chain constant domains, and a third polypeptide chain further comprising a second Fc region linked to C_{H1}, the second Fc region comprising an immunoglobulin hinge region and C_{H2} and C_{H3} immunoglobulin heavy chain constant domains; wherein the first and second Fc regions are human IgG4 Fc regions; and wherein the first and the second Fc regions each comprise amino acid substitutions at positions corresponding to positions 228 and 409 of human IgG4 according to EU Index, wherein the amino acid substitutions are S228P and R409K. In some embodiments, a binding protein of the present disclosure comprises knob and hole mutations and one or more mutations to improve stability. In some embodiments, the first and/or second Fc regions are human IgG4 Fc regions.

In some embodiments, a binding protein of the present disclosure comprises one or more mutations to improve purification, e.g., by modulating the affinity for a purification reagent. For example, it is known that heterodimeric binding proteins can be selectively purified away from their homodimeric forms if one of the two Fc regions of the heterodimeric form contains mutation(s) that reduce or eliminate binding to Protein A, because the heterodimeric form will have an intermediate affinity for Protein A-based purification than either homodimeric form and can be selectively eluted from Protein A, e.g., by use of a different pH (See e.g., Smith, E. J. et al. (2015) Sci. Rep. 5:17943). In some embodiments, the mutation comprises substitutions at positions corresponding to positions 435 and 436 of human IgG1 or IgG4 according to EU Index, wherein the amino acid substitutions are H435R and Y436F. In some embodiments, the binding protein comprises a second polypeptide chain further comprising a first Fc region linked to C_{H1}, the first Fc region comprising an immunoglobulin hinge region and C_{H2} and C_{H3} immunoglobulin heavy chain constant domains, and a third polypeptide chain further comprising a second Fc region linked to C_{H1}, the second Fc region comprising an immunoglobulin hinge region and C_{H2} and C_{H3} immunoglobulin heavy chain constant domains; and wherein only one of the first and the second Fc regions comprises amino acid substitutions at positions corresponding to positions 435 and 436 of human IgG1 or IgG4 according to EU Index, wherein the amino acid substitutions are H435R and Y436F. In some embodiments, a binding protein of the present disclosure comprises knob and hole mutations and one or more mutations to improve purification. In some embodiments, the first and/or second Fc regions are human IgG1 Fc regions. In some embodiments, the first and/or second Fc regions are human IgG4 Fc regions.

In some embodiments, a binding protein of the present disclosure comprises one or more mutations to reduce effector function, e.g., Fc receptor-mediated antibody-dependent cellular phagocytosis (ADCP), complement-dependent cytotoxicity (CDC), and/or antibody-dependent cellular cytotoxicity (ADCC). In some embodiments, the second polypeptide chain further comprises a first Fc region linked to C_{H1}, the first Fc region comprising an immunoglobulin hinge region and C_{H2} and C_{H3} immunoglobulin heavy chain constant domains; wherein the third polypeptide chain further comprises a second Fc region linked to C_{H1}, the second Fc region comprising an immunoglobulin hinge region and C_{H2} and C_{H3} immunoglobulin heavy chain constant domains; wherein the first and second Fc regions are human IgG1 Fc regions; and wherein the first and the second Fc regions each comprise amino acid substitutions at positions corresponding to positions 234 and 235 of human IgG1 according to EU Index, wherein the amino acid substitutions are L234A and L235A. In some embodiments, the Fc regions of the second and the third polypeptide chains are human IgG1 Fc regions, and wherein the Fc regions each comprise amino acid substitutions at positions corresponding to positions 234 and 235 of human IgG1 according to EU Index, wherein the amino acid substitutions are L234A and L235A. In some embodiments, the second polypeptide chain further comprises a first Fc region linked to C_{H1}, the first Fc region comprising an immunoglobulin hinge region and C_{H2} and C_{H3} immunoglobulin heavy chain constant domains; wherein the third polypeptide chain further comprises a second Fc region linked to C_{H1}, the second Fc region comprising an immunoglobulin hinge region and C_{H2} and C_{H3} immunoglobulin heavy chain constant domains; wherein the first and second Fc regions are human IgG1 Fc regions; and wherein the first and the second Fc regions each comprise amino acid substitutions at positions corresponding to positions 234, 235, 329 of human IgG1 according to EU Index, wherein the amino acid substitutions are L234A, L235A, and P329A. In some embodiments, the Fc regions of the second and the third polypeptide chains are human IgG1 Fc regions, and wherein the Fc regions each comprise amino acid substitutions at positions corresponding to positions 234, 235, and 329 of human IgG1 according to EU Index, wherein the amino acid substitutions are L234A, L235A, and P329A. In some embodiments, the mutation comprises substitutions at positions corresponding to positions 234 and 235 of human IgG4 according to EU Index, wherein the amino acid substitutions are F234A and L235A. In some embodiments, the binding protein comprises a second polypeptide chain further comprising a first Fc region linked to C_{H1}, the first Fc region comprising an immunoglobulin hinge region and C_{H2} and C_{H3} immunoglobulin heavy chain constant domains, and a third polypeptide chain further comprising a second Fc region linked to C_{H1}, the second Fc region comprising an immunoglobulin hinge region and C_{H2} and C_{H3} immunoglobulin heavy chain constant domains; and wherein the first and the second Fc regions each comprise amino acid substitutions at positions corresponding to positions 234 and 235 of human IgG4 according to EU Index, wherein the amino acid substitutions are F234A and L235A. In some embodiments, a binding protein of the present disclosure comprises knob and hole mutations and one or more mutations to reduce effector function. In some embodiments, the first and/or second Fc regions are human IgG1 Fc regions. In some embodiments, the first and/or second Fc regions are human IgG4 Fc regions. For further description of Fc mutations at position 329, see, e.g., Shields, R. L. et al. (2001) J. Biol. Chem. 276:6591-6604 and WO1999051642.

In some embodiments, the types of mutations described supra can be combined in any order or combination. For example, a binding protein of the present disclosure can comprise two or more of the “knob” and “hole” mutations, the one or more mutations to improve serum half-life, the one or more mutations to improve IgG4 stability, the one or more mutations to improve purification, and/or the one or more mutations to reduce effector function described supra.

In certain embodiments, a binding protein of the present disclosure comprises: a first polypeptide chain that comprises a lambda C_{L} domain; a C_{H3} domain of a second polypeptide chain that comprises amino acid substitutions at positions corresponding to positions 354 and 366 of human IgG1 according to EU Index, wherein the amino acid substitutions are S354C and T366W; a C_{H3} domain of a third polypeptide chain that comprises amino acid substitutions at positions corresponding to positions 349, 366, 368, 407, 435, and 436 of human IgG1 according to EU Index, wherein the amino acid substitutions are Y349C, T366S, L368A, Y407V, H435R, and Y436F; and a fourth polypeptide chain that comprises a kappa C_{L} domain. In some embodiments, the first polypeptide chain comprises a lambda C_{L} domain; wherein the C_{H3} domain of the third polypeptide chain comprises amino acid substitutions at positions corresponding to positions 354 and 366 of human IgG1 according to EU Index, wherein the amino acid substitutions are S354C and T366W; wherein the C_{H3} domain of the second polypeptide chain comprises amino acid substitutions at positions corresponding to positions 349, 366, 368, 407, 435, and 436 of human IgG1 according to EU Index, wherein the amino acid substitutions are Y349C, T366S, L368A, Y407V, H435R, and Y436F; and wherein the fourth polypeptide chain comprises a kappa C_{L} domain. In some embodiments, the first polypeptide chain comprises a lambda C_{L} domain; wherein the C_{H3} domain of the second polypeptide chain comprises amino acid substitutions at positions corresponding to positions 354, 366, 435, and 436 of human IgG1 according to EU Index, wherein the amino acid substitutions are S354C, T366W, H435R, and Y436F; wherein the C_{H3} domain of the third polypeptide chain comprises amino acid substitutions at positions corresponding to positions 349, 366, 368, and 407 of human IgG1 according to EU Index, wherein the amino acid substitutions are Y349C, T366S, L368A, and Y407V; and wherein the fourth polypeptide chain comprises a kappa C_{L} domain. In some embodiments, the first polypeptide chain comprises a kappa C_{L} domain; wherein the C_{H3} domain of the second polypeptide chain comprises amino acid substitutions at positions corresponding to positions 354 and 366 of human IgG1 according to EU Index, wherein the amino acid substitutions are S354C and T366W; wherein the C_{H3} domain of the third polypeptide chain comprises amino acid substitutions at positions corresponding to positions 349, 366, 368, 407, 435, and 436 of human IgG1 according to EU Index, wherein the amino acid substitutions are Y349C, T366S, L368A, Y407V, H435R, and Y436F; and wherein the fourth polypeptide chain comprises a lambda C_{L} domain.

In some embodiments, a binding protein of the present disclosure is purified by protein A affinity chromatography, kappa light chain affinity chromatography (e.g., using a KappaSelect resin according to manufacturer's instructions; GE Healthcare), and optionally lambda light chain affinity chromatography (e.g., using a LambdaFabSelect resin according to manufacturer's instructions; GE Healthcare). In some embodiments, a binding protein of the present disclosure is purified by Protein A affinity chromatography, lambda light chain affinity chromatography (e.g., using a LambdaFabSelect resin according to manufacturer's instructions; GE Healthcare), and optionally kappa light chain affinity chromatography (e.g., using a KappaSelect resin according to manufacturer's instructions; GE Healthcare). In some embodiments, the binding protein comprises two Fc regions, each comprising a C_{H3} domain, and only one of the C_{H3} domains comprises amino acid substitutions at positions corresponding to positions 435 and 436 of human IgG1 or IgG4 according to EU Index, wherein the amino acid substitutions are H435R and Y436F. In some embodiments, a binding protein of the present disclosure is purified by protein A affinity chromatography, then kappa light chain affinity chromatography (e.g., using a KappaSelect resin according to manufacturer's instructions; GE Healthcare), then optionally lambda light chain affinity chromatography (e.g., using a LambdaFabSelect resin according to manufacturer's instructions; GE Healthcare) in sequence. In some embodiments, a binding protein of the present disclosure is purified by Protein A affinity chromatography, then lambda light chain affinity chromatography (e.g., using a LambdaFabSelect resin according to manufacturer's instructions; GE Healthcare), then optionally kappa light chain affinity chromatography (e.g., using a KappaSelect resin according to manufacturer's instructions; GE Healthcare) in sequence. For example, in some embodiments, the binding protein is contacted with Protein A, eluted from Protein A under conditions suitable for isolating the binding protein away from binding proteins comprising either 0 or 2 C_{H3} domains comprising the amino acid substitutions are H435R and Y436F, contacted with a kappa light chain affinity medium (e.g., as used in the KappaSelect resin; GE Healthcare), and eluted from the kappa light chain affinity medium under conditions suitable for isolating the binding protein away from binding proteins comprising only lambda C_{L} domains (e.g., according to manufacturer's instructions). Conditions suitable for the Protein A elution are known in the art, including without limitation a stepwise elution gradient from pH4.5-2.8. In some embodiments, Protein A or a Protein A variant useful for protein purification is employed. In some embodiments, the Protein A is attached to a substrate or resin, e.g., as part of a chromatography medium. In some embodiments, after elution from the kappa light chain affinity medium, the binding protein is contacted with a lambda light chain affinity medium (e.g., as used in the LambdaFabSelect resin; GE Healthcare), and eluted from the lambda light chain affinity medium under conditions suitable for isolating the binding protein away from binding proteins comprising only kappa C_{L} domains (e.g., according to manufacturer's instructions). In some embodiments, a binding protein of the present disclosure is detected using HIC chromatography. In some embodiments, the binding protein comprises: a first polypeptide chain that comprises a lambda C_{L} domain; a C_{H3} domain of a second polypeptide chain that comprises amino acid substitutions at positions corresponding to positions 354 and 366 of human IgG1 or IgG4 according to EU Index, wherein the amino acid substitutions are S354C and T366W; a C_{H3} domain of a third polypeptide chain that comprises amino acid substitutions at positions corresponding to positions 349, 366, 368, 407, 435, and 436 of human IgG1 or IgG4 according to EU Index, wherein the amino acid substitutions are Y349C, T366S, L368A, Y407V, H435R, and Y436F; and a fourth polypeptide chain that comprises a kappa C_{L} domain. In some embodiments, the binding protein is produced by a host cell. In some embodiments, the binding protein is purified from a cell culture medium or host cell extract. In some embodiments, the binding proteins are secreted by a host cell or produced and extracted from a host cell (e.g., before being contacted with Protein A). In some embodiments, the binding protein is in a cell culture medium or host cell extract when contacted with Protein A. In some embodiments, the binding protein is purified away from other binding proteins, polypeptides, and/or other cellular components.

In some embodiments, CH1, CH2, CH3 and CL of the trispecific binding proteins described herein may comprise any of CH1, CH2, CH3 and CL sequences of binding proteins 1-53.

### Nucleic Acids

Standard recombinant DNA methodologies are used to construct the polynucleotides that encode the polypeptides which form the binding proteins, incorporate these polynucleotides into recombinant expression vectors, and introduce such vectors into host cells. See e.g., Sambrook et al., 2001, MOLECULAR CLONING: A LABORATORY MANUAL (Cold Spring Harbor Laboratory Press, 3rd ed.). Enzymatic reactions and purification techniques may be performed according to manufacturer's specifications, as commonly accomplished in the art, or as described herein. Unless specific definitions are provided, the nomenclature utilized in connection with, and the laboratory procedures and techniques of, analytical chemistry, synthetic organic chemistry, and medicinal and pharmaceutical chemistry described herein are those well-known and commonly used in the art. Similarly, conventional techniques may be used for chemical syntheses, chemical analyses, pharmaceutical preparation, formulation, delivery, and treatment of patients.

Other aspects of the present disclosure relate to isolated nucleic acid molecules comprising a nucleotide sequence encoding any of the binding proteins described herein. In some embodiments, the isolated nucleic acid is operably linked to a heterologous promoter to direct transcription of the binding protein-coding nucleic acid sequence. A promoter may refer to nucleic acid control sequences which direct transcription of a nucleic acid. A first nucleic acid sequence is operably linked to a second nucleic acid sequence when the first nucleic acid sequence is placed in a functional relationship with the second nucleic acid sequence. For instance, a promoter is operably linked to a coding sequence of a binding protein if the promoter affects the transcription or expression of the coding sequence. Examples of promoters may include, but are not limited to, promoters obtained from the genomes of viruses (such as polyoma virus, fowlpox virus, adenovirus (such as Adenovirus 2), bovine papilloma virus, avian sarcoma virus, cytomegalovirus, a retrovirus, hepatitis-B virus, Simian Virus 40 (SV40), and the like), from heterologous eukaryotic promoters (such as the actin promoter, an immunoglobulin promoter, from heat-shock promoters, and the like), the CAG-promoter (Niwa et al., Gene 108(2):193-9, 1991), the phosphoglycerate kinase (PGK)-promoter, a tetracycline-inducible promoter (Masui et al., Nucleic Acids Res. 33:e43, 2005), the lac system, the trp system, the tac system, the trc system, major operator and promoter regions of phage lambda, the promoter for 3-phosphoglycerate kinase, the promoters of yeast acid phosphatase, and the promoter of the yeast alpha-mating factors. Polynucleotides encoding binding proteins of the present disclosure may be under the control of a constitutive promoter, an inducible promoter, or any other suitable promoter described herein or other suitable promoter that will be readily recognized by one skilled in the art.

In some embodiments, the isolated nucleic acid is incorporated into a vector. In some embodiments, the vector is an expression vector. Expression vectors may include one or more regulatory sequences operatively linked to the polynucleotide to be expressed. The term “regulatory sequence” includes promoters, enhancers and other expression control elements (e.g., polyadenylation signals). Examples of suitable enhancers may include, but are not limited to, enhancer sequences from mammalian genes (such as globin, elastase, albumin, α-fetoprotein, insulin and the like), and enhancer sequences from a eukaryotic cell virus (such as SV40 enhancer on the late side of the replication origin (bp 100-270), the cytomegalovirus early promoter enhancer, the polyoma enhancer on the late side of the replication origin, adenovirus enhancers, and the like). Examples of suitable vectors may include, for example, plasmids, cosmids, episomes, transposons, and viral vectors (e.g., adenoviral, vaccinia viral, Sindbis-viral, measles, herpes viral, lentiviral, retroviral, adeno-associated viral vectors, etc.). Expression vectors can be used to transfect host cells, such as, for example, bacterial cells, yeast cells, insect cells, and mammalian cells. Biologically functional viral and plasmid DNA vectors capable of expression and replication in a host are known in the art, and can be used to transfect any cell of interest.

Other aspects of the present disclosure relate to a vector system comprising one or more vectors encoding a first, second, third, and fourth polypeptide chain of any of the binding proteins described herein. In some embodiments, the vector system comprises a first vector encoding the first polypeptide chain of the binding protein, a second vector encoding the second polypeptide chain of the binding protein, a third vector encoding the third polypeptide chain of the binding protein, and a fourth vector encoding the fourth polypeptide chain of the binding protein. In some embodiments, the vector system comprises a first vector encoding the first and second polypeptide chains of the binding protein, and a second vector encoding the third and fourth polypeptide chains of the binding protein. In some embodiments, the vector system comprises a first vector encoding the first and third polypeptide chains of the binding protein, and a second vector encoding the second and fourth polypeptide chains of the binding protein. In some embodiments, the vector system comprises a first vector encoding the first and fourth polypeptide chains of the binding protein, and a second vector encoding the second and third polypeptide chains of the binding protein. In some embodiments, the vector system comprises a first vector encoding the first, second, third, and fourth polypeptide chains of the binding protein. The one or more vectors of the vector system may be any of the vectors described herein. In some embodiments, the one or more vectors are expression vectors.

### Isolated Host Cells

Other aspects of the present disclosure relate to an isolated host cell comprising one or more isolated polynucleotides, vectors, and/or vector systems described herein. In some embodiments, the host cell is a bacterial cell (e.g., an E. coli cell). In some embodiments, the host cell is a yeast cell (e.g., an S. cerevisiae cell). In some embodiments, the host cell is an insect cell. Examples of insect host cells may include, for example, Drosophila cells (e.g., S2 cells), Trichoplusia ni cells (e.g., High Five™ cells), and Spodoptera frugiperda cells (e.g., Sf21 or Sf9 cells). In some embodiments, the host cell is a mammalian cell. Examples of mammalian host cells may include, for example, human embryonic kidney cells (e.g., 293 or 293 cells subcloned for growth in suspension culture), Expi293TM cells, CHO cells, baby hamster kidney cells (e.g., BHK, ATCC CCL 10), mouse sertoli cells (e.g., TM4 cells), monkey kidney cells (e.g., CV1 ATCC CCL 70), African green monkey kidney cells (e.g., VERO-76, ATCC CRL-1587), human cervical carcinoma cells (e.g., HELA, ATCC CCL 2), canine kidney cells (e.g., MDCK, ATCC CCL 34), buffalo rat liver cells (e.g., BRL 3A, ATCC CRL 1442), human lung cells (e.g., W138, ATCC CCL 75), human liver cells (e.g., Hep G2, HB 8065), mouse mammary tumor cells (e.g., MMT 060562, ATCC CCL51), TRI cells, MRC 5 cells, FS4 cells, a human hepatoma line (e.g., Hep G2), and myeloma cells (e.g., NS0 and Sp2/0 cells).

Other aspects of the present disclosure relate to a method of producing any of the binding proteins described herein. In some embodiments, the method includes a) culturing a host cell (e.g., any of the host cells described herein) comprising an isolated nucleic acid, vector, and/or vector system (e.g., any of the isolated nucleic acids, vectors, and/or vector systems described herein) under conditions such that the host cell expresses the binding protein; and b) isolating the binding protein from the host cell. Methods of culturing host cells under conditions to express a protein are well known to one of ordinary skill in the art. Methods of isolating proteins from cultured host cells are well known to one of ordinary skill in the art, including, for example, by affinity chromatography (e.g., two step affinity chromatography comprising protein A affinity chromatography followed by size exclusion chromatography).

### Uses for Binding Proteins

The binding proteins can be employed in any known assay method, such as competitive binding assays, direct and indirect sandwich assays, and immunoprecipitation assays for the detection and quantitation of one or more target antigens. The binding proteins will bind the one or more target antigens with an affinity that is appropriate for the assay method being employed.

For diagnostic applications, in certain embodiments, binding proteins can be labeled with a detectable moiety. The detectable moiety can be any one that is capable of producing, either directly or indirectly, a detectable signal. For example, the detectable moiety can be a radioisotope, such as ³H, ¹⁴C, ³²P, ³⁵S, ¹²⁵I, ⁹⁹Tc, ¹¹¹In, or ⁶⁷Ga; a fluorescent or chemiluminescent compound, such as fluorescein isothiocyanate, rhodamine, or luciferin; or an enzyme, such as alkaline phosphatase, β-galactosidase, or horseradish peroxidase.

The binding proteins are also useful for in vivo imaging. A binding protein labeled with a detectable moiety can be administered to an animal, preferably into the bloodstream, and the presence and location of the labeled antibody in the host assayed. The binding protein can be labeled with any moiety that is detectable in an animal, whether by nuclear magnetic resonance, radiology, or other detection means known in the art.

The binding proteins can also be used for cell activation, tumor targeting, neutralization of cytokine activities, neutralization of viral infection, combination of multiple signaling events, to treat cancer, arthritis, and/or inflammatory disorders. For example, in some embodiments, a binding protein specifically binds one, two, or three antigen targets selected from A2AR, APRIL, ATPDase, BAFF, BAFFR, BCMA, BlyS, BTK, BTLA, B7DC, B7H1, B7H4 (also known as VTCN1), B7H5, B7H6, B7H7, B7RP1, B7-4, C3, C5, CCL2 (also known as MCP-1), CCL3 (also known as MIP-1a), CCL4 (also known as MIP-1b), CCL5 (also known as RANTES), CCL7 (also known as MCP-3), CCL8 (also known as mcp-2), CCL11 (also known as eotaxin), CCL15 (also known as MIP-1d), CCL17 (also known as TARC), CCL19 (also known as MIP-3b), CCL20 (also known as MIP-3a), CCL21 (also known as MIP-2), CCL24 (also known as MPIF-2/eotaxin-2), CCL25 (also known as TECK), CCL26 (also known as eotaxin-3), CCR3, CCR4, CD3, CD19, CD20, CD23 (also known as FCER2, a receptor for IgE), CD24, CD27, CD28, CD38, CD39, CD40, CD70, CD80 (also known as B7-1), CD86 (also known as B7-2), CD122, CD137 (also known as 41BB), CD137L, CD152 (also known as CTLA4), CD154 (also known as CD40L), CD160, CD272, CD273 (also known as PDL2), CD274 (also known as PDL1), CD275 (also known as B7H2), CD276 (also known as B7H3), CD278 (also known as ICOS), CD279 (also known as PD-1), CDH1 (also known as E-cadherin), chitinase, CLEC9, CLEC91, CRTH2, CSF-1 (also known as M-CSF), CSF-2 (also known as GM-CSF), CSF-3 (also known as GCSF), CX3CL1 (also known as SCYD1), CXCL12 (also known as SDF1), CXCL13, CXCR3, DNGR-1, ectonucleoside triphosphate diphosphohydrolase 1, EGFR, ENTPD1, FCER1A, FCER1, FLAP, FOLH1, Gi24, GITR, GITRL, GM-CSF, Her2, HHLA2, HMGB1, HVEM, ICOSLG, IDO, IFNα, IgE, IGF1R, IL2Rbeta, IL1, IL1A, IL1B, IL1F10, IL2, IL4, IL4Ra, IL5, IL5R, IL6, IL7, IL7Ra, IL8, IL9, IL9R, IL10, rhIL10, IL12, IL13, IL13Ra1, IL13Ra2, IL15, IL17, IL17Rb (also known as a receptor for IL25), IL18, IL22, IL23, IL25, IL27, IL33, IL35, ITGB4 (also known as b4 integrin), ITK, KIR, LAG3, LAMP1, leptin, LPFS2, MHC class II, NCR3LG1, NKG2D, NTPDase-1, OX40, OX40L, PD-1H, platelet receptor, PROM1, S152, SISP1, SLC, SPG64, ST2 (also known as a receptor for IL33), STEAP2, Syk kinase, TACI, TDO, T14, TIGIT, TIM3, TLR, TLR2, TLR4, TLR5, TLR9, TMEF1, TNFa, TNFRSF7, Tp55, TREM1, TSLP (also known as a co-receptor for IL7Ra), TSLPR, TWEAK, VEGF, VISTA, Vstm3, WUCAM, and XCR1 (also known as GPR5/CCXCR1). In some embodiments, one or more of the above antigen targets are human antigen targets.

In some embodiments, a binding protein of the present disclosure is adminstered to a patient in need thereof for the treatment or prevention of cancer. For example, in some embodiments, the binding protein comprises one antigen binding site that specifically binds a T-cell surface protein and another antigen binding site that specifically binds a tumor target protein (e.g., two antigen binding sites that specifically bind T-cell surface proteins and one antigen binding site that specifically binds a tumor target protein, or two antigen binding sites that specifically bind tumor target proteins and one antigen binding site that specifically binds a T-cell surface protein). In certain embodiments, the binding protein comprises an antigen binding site that specifically binds CD3, an antigen binding site that specifically binds CD28, and an antigen binding site that specifically binds a tumor target protein selected from CD19, CD20, CD38, Her2, and LAMP1. In some embodiments, the binding protein is co-administered with a chemotherapeutic agent. In some embodiments, the patient is a human.

In some embodiments, a binding protein of the present disclosure is adminstered to a patient in need thereof for the treatment or prevention of an inflammatory disease or disorder. In some embodiments, the binding protein comprises three antigen binding sites that each specifically bind a cytokine target protein selected from IL-4, IL-13 and TNFa. In some embodiments, the binding protein is co-administered with an anti-inflammatory agent. In some embodiments, the patient is a human.

The disclosure also relates to a kit comprising a binding protein and other reagents useful for detecting target antigen levels in biological samples. Such reagents can include a detectable label, blocking serum, positive and negative control samples, and detection reagents. In some embodiments, the kit comprises a composition comprising any binding protein, polynucleotide, vector, vector system, and/or host cell described herein. In some embodiments, the kit comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing a condition and may have a sterile access port (for example the container may be an intravenous solution bag or a vial having a stopper pierceable by a hypodermic injection needle). In some embodiments, the label or package insert indicates that the composition is used for preventing, diagnosing, and/or treating the condition of choice. Alternatively, or additionally, the article of manufacture or kit may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Binding Protein Therapeutic Compositions and Administration Thereof

Therapeutic or pharmaceutical compositions comprising binding proteins are within the scope of the disclosure. Such therapeutic or pharmaceutical compositions can comprise a therapeutically effective amount of a binding protein, or binding protein-drug conjugate, in admixture with a pharmaceutically or physiologically acceptable formulation agent selected for suitability with the mode of administration.

Acceptable formulation materials preferably are nontoxic to recipients at the dosages and concentrations employed.

The pharmaceutical composition can contain formulation materials for modifying, maintaining, or preserving, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides—preferably sodium or potassium chloride—or mannitol sorbitol), delivery vehicles, diluents, excipients and/or pharmaceutical adjuvants (see, e.g., REMINGTON'S PHARMACEUTICAL SCIENCES (18th Ed., A. R. Gennaro, ed., Mack Publishing Company 1990), and subsequent editions of the same, incorporated herein by reference for any purpose).

The optimal pharmaceutical composition will be determined by a skilled artisan depending upon, for example, the intended route of administration, delivery format, and desired dosage. Such compositions can influence the physical state, stability, rate of in vivo release, and rate of in vivo clearance of the binding protein.

The primary vehicle or carrier in a pharmaceutical composition can be either aqueous or non-aqueous in nature. For example, a suitable vehicle or carrier for injection can be water, physiological saline solution, or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which can further include sorbitol or a suitable substitute. In one embodiment of the disclosure, binding protein compositions can be prepared for storage by mixing the selected composition having the desired degree of purity with optional formulation agents in the form of a lyophilized cake or an aqueous solution. Further, the binding protein can be formulated as a lyophilizate using appropriate excipients such as sucrose.

The pharmaceutical compositions of the disclosure can be selected for parenteral delivery or subcutaneous. Alternatively, the compositions can be selected for inhalation or for delivery through the digestive tract, such as orally. The preparation of such pharmaceutically acceptable compositions is within the skill of the art.

The formulation components are present in concentrations that are acceptable to the site of administration. For example, buffers are used to maintain the composition at physiological pH or at a slightly lower pH, typically within a pH range of from about 5 to about 8.

When parenteral administration is contemplated, the therapeutic compositions for use can be in the form of a pyrogen-free, parenterally acceptable, aqueous solution comprising the desired binding protein in a pharmaceutically acceptable vehicle. A particularly suitable vehicle for parenteral injection is sterile distilled water in which a binding protein is formulated as a sterile, isotonic solution, properly preserved. Yet another preparation can involve the formulation of the desired molecule with an agent, such as injectable microspheres, bio-erodible particles, polymeric compounds (such as polylactic acid or polyglycolic acid), beads, or liposomes, that provides for the controlled or sustained release of the product which can then be delivered via a depot injection. Hyaluronic acid can also be used, and this can have the effect of promoting sustained duration in the circulation. Other suitable means for the introduction of the desired molecule include implantable drug delivery devices.

In one embodiment, a pharmaceutical composition can be formulated for inhalation. For example, a binding protein can be formulated as a dry powder for inhalation. Binding protein inhalation solutions can also be formulated with a propellant for aerosol delivery. In yet another embodiment, solutions can be nebulized.

It is also contemplated that certain formulations can be administered orally. In one embodiment of the disclosure, binding proteins that are administered in this fashion can be formulated with or without those carriers customarily used in the compounding of solid dosage forms such as tablets and capsules. For example, a capsule can be designed to release the active portion of the formulation at the point in the gastrointestinal tract when bioavailability is maximized and pre-systemic degradation is minimized. Additional agents can be included to facilitate absorption of the binding protein. Diluents, flavorings, low melting point waxes, vegetable oils, lubricants, suspending agents, tablet disintegrating agents, and binders can also be employed.

Another pharmaceutical composition can involve an effective quantity of binding proteins in a mixture with non-toxic excipients that are suitable for the manufacture of tablets. By dissolving the tablets in sterile water, or another appropriate vehicle, solutions can be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions of the disclosure will be evident to those skilled in the art, including formulations involving binding proteins in sustained- or controlled-delivery formulations. Techniques for formulating a variety of other sustained- or controlled-delivery means, such as liposome carriers, bio-erodible microparticles or porous beads and depot injections, are also known to those skilled in the art. Additional examples of sustained-release preparations include semipermeable polymer matrices in the form of shaped articles, e.g. films, or microcapsules. Sustained release matrices can include polyesters, hydrogels, polylactides, copolymers of L-glutamic acid and gamma ethyl-L-glutamate, poly(2-hydroxyethyl-methacrylate), ethylene vinyl acetate, or poly-D(−)-3-hydroxybutyric acid. Sustained-release compositions can also include liposomes, which can be prepared by any of several methods known in the art.

Pharmaceutical compositions to be used for in vivo administration typically must be sterile. This can be accomplished by filtration through sterile filtration membranes. Where the composition is lyophilized, sterilization using this method can be conducted either prior to, or following, lyophilization and reconstitution. The composition for parenteral administration can be stored in lyophilized form or in a solution. In addition, parenteral compositions generally are placed into a container having a sterile access port, for example, an intravenous solution bag or vial having a stopper pierceable by a hypodermic injection needle.

Once the pharmaceutical composition has been formulated, it can be stored in sterile vials as a solution, suspension, gel, emulsion, solid, or as a dehydrated or lyophilized powder. Such formulations can be stored either in a ready-to-use form or in a form (e.g., lyophilized) requiring reconstitution prior to administration.

The disclosure also encompasses kits for producing a single-dose administration unit. The kits can each contain both a first container having a dried protein and a second container having an aqueous formulation. Also included within the scope of this disclosure are kits containing single and multi-chambered pre-filled syringes (e.g., liquid syringes and lyosyringes).

The effective amount of a binding protein pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the binding protein is being used, the route of administration, and the size (body weight, body surface, or organ size) and condition (the age and general health) of the patient. Accordingly, the clinician can titer the dosage and modify the route of administration to obtain the optimal therapeutic effect.

Dosing frequency will depend upon the pharmacokinetic parameters of the binding protein in the formulation being used. Typically, a clinician will administer the composition until a dosage is reached that achieves the desired effect. The composition can therefore be administered as a single dose, as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages can be ascertained through use of appropriate dose-response data.

The route of administration of the pharmaceutical composition is in accord with known methods, e.g., orally; through injection by intravenous, intraperitoneal, intracerebral (intraparenchymal), intracerebroventricular, intramuscular, intraocular, intraarterial, intraportal, or intralesional routes; by sustained release systems; or by implantation devices. Where desired, the compositions can be administered by bolus injection or continuously by infusion, or by implantation device.

The composition can also be administered locally via implantation of a membrane, sponge, or other appropriate material onto which the desired molecule has been absorbed or encapsulated. Where an implantation device is used, the device can be implanted into any suitable tissue or organ, and delivery of the desired molecule can be via diffusion, timed-release bolus, or continuous administration.

In some embodiments, the present disclosure relates to a method of preventing and/or treating a proliferative disease or disorder (e.g., cancer). In some embodiments, the method comprises administering to a patient a therapeutically effective amount of at least one of the binding proteins described herein. In some embodiments, the patient is a human. In some embodiments, the at least one binding protein is administered in combination with one or more anti-cancer therapies (e.g., any anti-cancer therapy known in the art). In some embodiments, the at least one binding protein is administered before the one or more anti-cancer therapies. In some embodiments, the at least one binding protein is administered concurrently with the one or more anti-cancer therapies. In some embodiments, the at least one binding protein is administered after the one or more anti-retroviral therapies.

In some embodiments, the present disclosure relates to a method of preventing and/or treating an inflammatory disease or disorder (e.g., cancer). In some embodiments, the method comprises administering to a patient a therapeutically effective amount of at least one of the binding proteins described herein. In some embodiments, the patient is a human. In some embodiments, the at least one binding protein is administered in combination with one or more anti-inflammatory therapies (e.g., any anti-inflammatory therapy known in the art). In some embodiments, the at least one binding protein is administered before the one or more anti-inflammatory therapies. In some embodiments, the at least one binding protein is administered concurrently with the one or more anti-inflammatory therapies. In some embodiments, the at least one binding protein is administered after the one or more anti-inflammatory therapies.

Without limiting the present disclosure, a number of embodiments of the present disclosure are described below for purpose of illustration.

Item 1: A binding protein comprising four polypeptide chains that form three antigen binding sites that specifically bind one or more target proteins, wherein a first polypeptide chain comprises a structure represented by the formula:

V_{L2}-L₁-V_{L1}-L₂-C_{L} [I]

and a second polypeptide chain comprises a structure represented by the formula:

V_{H1}-L₃-V_{H2}-L₄-C_{H1} [II]

and a third polypeptide chain comprises a structure represented by the formula:

V_{H3}-C_{H1} [III]

and a fourth polypeptide chain comprises a structure represented by the formula:

V_{L3}-C_{L} [IV]

wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain; and
L₁, L₂, L₃ and L₄ are amino acid linkers;
   and wherein the polypeptide of formula I and the polypeptide of formula II form a cross-over light chain-heavy chain pair.

Item 2: A binding protein comprising four polypeptide chains that form three antigen binding sites that specifically bind one or more target proteins, wherein a first polypeptide chain comprises a structure represented by the formula:

V_{L2}-L₁-V_{L1}-L₂-C_{L} [I]

and a second polypeptide chain comprises a structure represented by the formula:

V_{H1}-L₃-V_{H2}-L₄-C_{H1}-hinge-C_{H2}-C_{H3} [II]

and a third polypeptide chain comprises a structure represented by the formula:

V_{H3}-C_{H1}-hinge-C_{H2}-C_{H3} [III]

and a fourth polypeptide chain comprises a structure represented by the formula:

V_{L3}-C_{L} [IV]

wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain;
C_{H2} is an immunoglobulin C_{H2} heavy chain constant domain;
C_{H3} is an immunoglobulin C_{H3} heavy chain constant domain;
hinge is an immunoglobulin hinge region connecting the C_{H1} and C_{H2} domains; and
L₁, L₂, L₃ and L₄ are amino acid linkers;
and wherein the polypeptide of formula I and the polypeptide of formula II form a cross-over light chain-heavy chain pair.

Item 3: The binding protein of item 1, wherein the second and/or the third polypeptide chain further comprises an Fc region linked to C_{H1}, the Fc region comprising an immunoglobulin hinge region and C_{H2} and C_{H3} immunoglobulin heavy chain constant domains.

Item 4: The binding protein of any one of items 1-3, wherein at least one of L₁, L₂, L₃ or L₄ is independently 0 amino acids in length.

Item 5: The binding protein of any one of items 1-3, wherein L₁, L₂, L₃ or L₄ are each independently at least one amino acid in length.

Item 6: The binding protein of any one of items 1-3 and 5, wherein (a) L₁, L₂, L₃ and L₄ each independently are zero amino acids in length or comprise a sequence selected from the group consisting of GGGGSGGGGS (SEQ ID NO:104), GGGGSGGGGSGGGGS (SEQ ID NO:105), S, RT, TKGPS (SEQ ID NO:106), GQPKAAP (SEQ ID NO: 175), and GGSGSSGSGG (SEQ ID NO:148); or (b) L₁, L₂, L₃ and L₄ each independently comprise a sequence selected from the group consisting of GGGGSGGGGS (SEQ ID NO:104), GGGGSGGGGSGGGGS (SEQ ID NO:105), S, RT, TKGPS (SEQ ID NO:106), GQPKAAP (SEQ ID NO: 175), and GGSGSSGSGG (SEQ ID NO:148).

Item 7: The binding protein of any one of items 1-5, wherein
(a) L₁ comprises the sequence GQPKAAP (SEQ ID NO: 175), L₂ comprises the sequence TKGPS (SEQ ID NO:106), L₃ comprises the sequence S, and L₄ comprises the sequence RT;
(b) L₁ comprises the sequence GGGGSGGGGS (SEQ ID NO:104), L₂ comprises the sequence GGGGSGGGGS (SEQ ID NO:104), L₃ is 0 amino acids in length, and L₄ is 0 amino acids in length;
(c) L₁ comprises the sequence GGSGSSGSGG (SEQ ID NO:148), L₂ comprises the sequence GGSGSSGSGG (SEQ ID NO:148), L₃ is 0 amino acids in length, and L₄ is 0 amino acids in length; or
(d) L₁ comprises the sequence GGGGSGGGGSGGGGS (SEQ ID NO:105), L₂ is 0 amino acids in length, L₃ comprises the sequence GGGGSGGGGSGGGGS (SEQ ID NO:105), and L₄ is 0 amino acids in length.

Item 8: The binding protein of any one of items 1-7, wherein the binding protein is trispecific and capable of specifically binding three different antigen targets.

Item 9: The binding protein of any one of items 1-8, wherein the binding protein specifically binds three target proteins that correspond to two target proteins on T cells and to one tumor target protein.

Item 10: The binding protein of item 9, wherein one of said target proteins on T cells is CD3.

Item 11: The binding protein of item 9 or item 10, wherein one of said target proteins on T cells is CD28.

Item 12: The binding protein of any one of items 9-11, wherein said tumor target protein is CD38.

Item 13: The binding protein of any one of items 1-8, wherein the binding protein specifically binds three target proteins that correspond to two target proteins on T cells and to one target protein selected from the group consisting of A2AR, APRIL, ATPDase, BAFF, BAFFR, BCMA, BlyS, BTK, BTLA, B7DC, B7H1, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL24, CCL25, CCL26, CCR3, CCR4, CD3, CD19, CD20, CD23, CD24, CD27, CD28, CD38, CD39, CD40, CD70, CD80, CD86, CD122, CD137, CD137L, CD152, CD154, CD160, CD272, CD273, CD274, CD275, CD276, CD278, CD279, CDH1, chitinase, CLEC9, CLEC91, CRTH2, CSF-1, CSF-2, CSF-3, CX3CL1, CXCL12, CXCL13, CXCR3, DNGR-1, ectonucleoside triphosphate diphosphohydrolase 1, EGFR, ENTPD1, FCER1A, FCER1, FLAP, FOLH1, Gi24, GITR, GITRL, GM-CSF, Her2, HHLA2, HMGB1, HVEM, ICOSLG, IDO, IFNα, IgE, IGF1R, IL2Rbeta, IL1, IL1A, IL1B, IL1F10, IL2, IL4, IL4Ra, IL5, IL5R, IL6, IL7, IL7Ra, IL8, IL9, IL9R, IL10, rhIL10, IL12, IL13, IL13Ra1, IL13Ra2, IL15, IL17, IL17Rb, IL18, IL22, IL23, IL25, IL27, IL33, IL35, ITGB4, ITK, KIR, LAG3, LAMP1, leptin, LPFS2, MHC class II, NCR3LG1, NKG2D, NTPDase-1, OX40, OX40L, PD-1H, platelet receptor, PROM1, S152, SISP1, SLC, SPG64, ST2, STEAP2, Syk kinase, TACI, TDO, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, WUCAM, and XCR1.

Item 14: The binding protein of any one of items 1-13, wherein the binding protein is capable of inhibiting the function of one or more target proteins.

Item 15: The binding protein of item 14, wherein the one or more target proteins are selected from the group consisting of A2AR, APRIL, ATPDase, BAFF, BAFFR, BCMA, BlyS, BTK, BTLA, B7DC, B7H1, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL24, CCL25, CCL26, CCR3, CCR4, CD3, CD19, CD20, CD23, CD24, CD27, CD28, CD38, CD39, CD40, CD70, CD80, CD86, CD122, CD137, CD137L, CD152, CD154, CD160, CD272, CD273, CD274, CD275, CD276, CD278, CD279, CDH1, chitinase, CLEC9, CLEC91, CRTH2, CSF-1, CSF-2, CSF-3, CX3CL1, CXCL12, CXCL13, CXCR3, DNGR-1, ectonucleoside triphosphate diphosphohydrolase 1, EGFR, ENTPD1, FCER1A, FCER1, FLAP, FOLH1, Gi24, GITR, GITRL, GM-CSF, Her2, HHLA2, HMGB1, HVEM, ICOSLG, IDO, IFNα, IgE, IGF1R, IL2Rbeta, IL1, IL1A, IL1B, IL1F10, IL2, IL4, IL4Ra, IL5, IL5R, IL6, IL7, IL7Ra, IL8, IL9, IL9R, IL10, rhIL10, IL12, IL13, IL13Ra1, IL13Ra2, IL15, IL17, IL17Rb, IL18, IL22, IL23, IL25, IL27, IL33, IL35, ITGB4, ITK, KIR, LAG3, LAMP1, leptin, LPFS2, MHC class II, NCR3LG1, NKG2D, NTPDase-1, OX40, OX40L, PD-1H, platelet receptor, PROM1, S152, SISP1, SLC, SPG64, ST2, STEAP2, Syk kinase, TACI, TDO, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, WUCAM, and XCR1.

Item 16: A binding protein comprising four polypeptide chains that form three antigen binding sites, wherein a first polypeptide chain comprises a structure represented by the formula:

V_{L2}-L₁-V_{L1}-L₂-C_{L} [I]

and a second polypeptide chain comprises a structure represented by the formula:

V_{H1}-L₃-V_{H2}-L₄-C_{H1} [II]

and a third polypeptide chain comprises a structure represented by the formula:

V_{H3}-C_{H1} [III]

and a fourth polypeptide chain comprises a structure represented by the formula:

V_{L3}-C_{L} [IV]

wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain; and
L₁, L₂, L₃ and L₄ are amino acid linkers;
wherein the polypeptide of formula I and the polypeptide of formula II form a cross-over light chain-heavy chain pair;
wherein:
(a) V_{L1}, V_{L2} and V_{L3} each independently comprise a variable domain sequence as set forth in any one of SEQ ID NOs:151, 153, 155, 157, 159, 161, 163, 165, and 167; or
(b) V_{L1}, V_{L2} and V_{L3} each independently comprise light chain complementarity determining regions comprising an amino acid sequence as set forth in any one of SEQ ID NOs: 43-59, 123-125, 138-140, and 149; and
wherein:
(a) V_{H1}, V_{H2}, and V_{H3} each independently comprise a variable domain sequence as set forth in any one of SEQ ID NOs:150, 152, 154, 156, 158, 160, 162, 164, and 166; or
(b) V_{H1}, V_{H2} and V_{H3} each independently comprise heavy chain complementarity determining regions comprising an amino acid sequence as set forth in any one of SEQ ID NOs: 25-42, 120-122, and 126-128.

Item 17: A binding protein comprising four polypeptide chains that form three antigen binding sites, wherein a first polypeptide chain comprises a structure represented by the formula:

V_{L2}-L₁-V_{L1}-L₂-C_{L} [I]

and a second polypeptide chain comprises a structure represented by the formula:

V_{H1}-L₃-V_{H2}-L₄-C_{H1}-hinge-C_{H2}-C_{H3} [II]

and a third polypeptide chain comprises a structure represented by the formula:

V_{H3}-C_{H1}-hinge-CH₁-CH₃ [III]

and a fourth polypeptide chain comprises a structure represented by the formula:

V_{L3}-C_{L} [IV]

wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain;
C_{H2} is an immunoglobulin C_{H2} heavy chain constant domain;
C_{H3} is an immunoglobulin C_{H3} heavy chain constant domain;
hinge is an immunoglobulin hinge region connecting the CHM and C_{H2} domains; and
L₁, L₂, L₃ and L₄ are amino acid linkers;
wherein the polypeptide of formula I and the polypeptide of formula II form a cross-over light chain-heavy chain pair;
wherein:
(a) V_{L1}, V_{L2} and V_{L3} each independently comprise a variable domain sequence as set forth in any one of SEQ ID NOs:151, 153, 155, 157, 159, 161, 163, 165, and 167; or
(b) V_{L1}, V_{L2} and V_{L3} each independently comprise light chain complementarity determining regions comprising an amino acid sequence as set forth in any one of SEQ ID NOs: 43-59, 123-125, 138-140, and 149; and wherein:
(a) V_{H1}, V_{H2}, and V_{H3} each independently comprise a variable domain sequence as set forth in any one of SEQ ID NOs:150, 152, 154, 156, 158, 160, 162, 164, and 166; or
(b) V_{H1}, V_{H2} and V_{H3} each independently comprise heavy chain complementarity determining regions comprising an amino acid sequence as set forth in any one of SEQ ID NOs: 25-42, 120-122, and 126-128.

Item 18: The binding protein of item 16, wherein the second and/or the third polypeptide chain further comprises an Fc region linked to C_{H1}, the Fc region comprising an immunoglobulin hinge region and C_{H2} and C_{H3} immunoglobulin heavy chain constant domains.

Item 19: The binding protein of any one of items 16-18, wherein at least one of L₁, L₂, L₃ or L₄, is independently 0 amino acids in length.

Item 20: The binding protein of any one of items 16-18, wherein L₁, L₂, L₃ or L₄ are each independently at least one amino acid in length.

Item 21: The binding protein of any one of items 16-18 and 20, wherein (a) L₁, L₂, L₃ and L₄ each independently are zero amino acids in length or comprise a sequence selected from the group consisting of GGGGSGGGGS (SEQ ID NO:104), GGGGSGGGGSGGGGS (SEQ ID NO:105), S, RT, TKGPS (SEQ ID NO:106), GQPKAAP (SEQ ID NO: 175), and GGSGSSGSGG (SEQ ID NO:148); or (b) L₁, L₂, L₃ and L₄ each independently comprise a sequence selected from the group consisting of GGGGSGGGGS (SEQ ID NO:104), GGGGSGGGGSGGGGS (SEQ ID NO:105), S, RT, TKGPS (SEQ ID NO:106), GQPKAAP (SEQ ID NO: 175), and GGSGSSGSGG (SEQ ID NO:148).

Item 22: The binding protein of any one of items 16-20, wherein:
(a) L₁ comprises the sequence GQPKAAP (SEQ ID NO: 175), L₂ comprises the sequence TKGPS (SEQ ID NO:106), L₃ comprises the sequence S, and L₄ comprises the sequence RT;
(b) L₁ comprises the sequence GGGGSGGGGS (SEQ ID NO:104), L₂ comprises the sequence GGGGSGGGGS (SEQ ID NO:104), L₃ is 0 amino acids in length, and L₄ is 0 amino acids in length;
(c) L₁ comprises the sequence GGSGSSGSGG (SEQ ID NO:148), L₂ comprises the sequence GGSGSSGSGG (SEQ ID NO:148), L₃ is 0 amino acids in length, and L₄ is 0 amino acids in length; or
(d) L₁ comprises the sequence GGGGSGGGGSGGGGS (SEQ ID NO:105), L₂ is 0 amino acids in length, L₃ comprises the sequence GGGGSGGGGSGGGGS (SEQ ID NO:105), and L₄ is 0 amino acids in length.

Item 23: The binding protein of any one of items 16-22, wherein:
(a) V_{H1} comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:30; V_{L1} comprises a CDR-L1 comprising the amino acid sequence of SEQ ID NO:46, a CDR-L2 comprising the amino acid sequence of SEQ ID NO: 47, and a CDR-L3 comprising the amino acid sequence of SEQ ID NO:48; V_{H2} Comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} comprises a CDR-H1 comprising the amino acid sequence of SEQ ID NO:25, a CDR-H2 comprising the amino acid sequence of SEQ ID NO:26, and a CDR-H3 comprising the amino acid sequence of SEQ ID NO:27; and V_{L3} comprises a CDR-L1 comprising the amino acid sequence of SEQ ID NO:43, a CDR-L2 comprising the amino acid sequence of SEQ ID NO:44, and a CDR-L3 comprising the amino acid sequence of SEQ ID NO:45;
(b) V_{H1} comprises a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33; V_{L1} comprises a CDR-L1 comprising the amino acid sequence of SEQ ID NO:49, a CDR-L2 comprising the amino acid sequence of SEQ ID NO:50, and a CDR-L3 comprising the amino acid sequence of SEQ ID NO:51; V_{H2} Comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} comprises a CDR-H1 comprising the amino acid sequence of SEQ ID NO:25, a CDR-H2 comprising the amino acid sequence of SEQ ID NO:26, and a CDR-H3 comprising the amino acid sequence of SEQ ID NO:27; and V_{L3} comprises a CDR-L1 comprising the amino acid sequence of SEQ ID NO:43, a CDR-L2 comprising the amino acid sequence of SEQ ID NO:44, and a CDR-L3 comprising the amino acid sequence of SEQ ID NO:45;
(c) V_{H1} comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:30; V_{L1} comprises a CDR-L1 comprising the amino acid sequence of SEQ ID NO:46, a CDR-L2 comprising the amino acid sequence of SEQ ID NO: 47, and a CDR-L3 comprising the amino acid sequence of SEQ ID NO:48; V_{H2} comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} comprises a CDR-H1 comprising the amino acid sequence of SEQ ID NO:37, a CDR-H2 comprising the amino acid sequence of SEQ ID NO:38, and a CDR-H3 comprising the amino acid sequence of SEQ ID NO:39; and V_{L3} comprises a CDR-L1 comprising the amino acid sequence of SEQ ID NO:55, a CDR-L2 comprising the amino acid sequence of SEQ ID NO:56, and a CDR-L3 comprising the amino acid sequence of SEQ ID NO:57;
(d) V_{H1} comprises a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33; V_{L1} comprises a CDR-L1 comprising the amino acid sequence of SEQ ID NO:49, a CDR-L2 comprising the amino acid sequence of SEQ ID NO:50, and a CDR-L3 comprising the amino acid sequence of SEQ ID NO:51; V_{H2} Comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} comprises a CDR-H1 comprising the amino acid sequence of SEQ ID NO:37, a CDR-H2 comprising the amino acid sequence of SEQ ID NO:38, and a CDR-H3 comprising the amino acid sequence of SEQ ID NO:39; and V_{L3} comprises a CDR-L1 comprising the amino acid sequence of SEQ ID NO:55, a CDR-L2 comprising the amino acid sequence of SEQ ID NO:56, and a CDR-L3 comprising the amino acid sequence of SEQ ID NO:57;
(e) V_{H1} comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:30; V_{L1} comprises a CDR-L1 comprising the amino acid sequence of SEQ ID NO:46, a CDR-L2 comprising the amino acid sequence of SEQ ID NO:179, and a CDR-L3 comprising the amino acid sequence of SEQ ID NO:48; V_{H2} comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} Comprises a CDR-H1 comprising the amino acid sequence of SEQ ID NO:40, a CDR-H2 comprising the amino acid sequence of SEQ ID NO:41, and a CDR-H3 comprising the amino acid sequence of SEQ ID NO:42; and V_{L3} comprises a CDR-L1 comprising the amino acid sequence of SEQ ID NO:58, a CDR-L2 comprising the amino acid sequence of SEQ ID NO:44, and a CDR-L3 comprising the amino acid sequence of SEQ ID NO:59;
(f) V_{H1} comprises a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33; V_{L1} comprises a CDR-L1 comprising the amino acid sequence of SEQ ID NO:49, a CDR-L2 comprising the amino acid sequence of SEQ ID NO:50, and a CDR-L3 comprising the amino acid sequence of SEQ ID NO:51; V_{H2} Comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} comprises a CDR-H1 comprising the amino acid sequence of SEQ ID NO:40, a CDR-H2 comprising the amino acid sequence of SEQ ID NO:41, and a CDR-H3 comprising the amino acid sequence of SEQ ID NO:42; and V_{L3} comprises a CDR-L1 comprising the amino acid sequence of SEQ ID NO:58, a CDR-L2 comprising the amino acid sequence of SEQ ID NO:44, and a CDR-L3 comprising the amino acid sequence of SEQ ID NO:59;
(g) V_{H1} comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:30; V_{L1} comprises a CDR-L1 comprising the amino acid sequence of SEQ ID NO:46, a CDR-L2 comprising the amino acid sequence of SEQ ID NO: 47, and a CDR-L3 comprising the amino acid sequence of SEQ ID NO:48; V_{H2} comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} comprises a CDR-H1 comprising the amino acid sequence of SEQ ID NO:126, a CDR-H2 comprising the amino acid sequence of SEQ ID NO:127, and a CDR-H3 comprising the amino acid sequence of SEQ ID NO:128; and V_{L3} comprises a CDR-L1 comprising the amino acid sequence of SEQ ID NO:138, a CDR-L2 comprising the amino acid sequence of SEQ ID NO:139, and a CDR-L3 comprising the amino acid sequence of SEQ ID NO:140;
(h) V_{H1} comprises a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33; V_{L1} comprises a CDR-L1 comprising the amino acid sequence of SEQ ID NO:49, a CDR-L2 comprising the amino acid sequence of SEQ ID NO:50, and a CDR-L3 comprising the amino acid sequence of SEQ ID NO:51; V_{H2} Comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} comprises a CDR-H1 comprising the amino acid sequence of SEQ ID NO:126, a CDR-H2 comprising the amino acid sequence of SEQ ID NO:127, and a CDR-H3 comprising the amino acid sequence of SEQ ID NO:128; and V_{L3} comprises a CDR-L1 comprising the amino acid sequence of SEQ ID NO:138, a CDR-L2 comprising the amino acid sequence of SEQ ID NO:139, and a CDR-L3 comprising the amino acid sequence of SEQ ID NO:140;
(i) V_{H1} comprises a CDR-H1 comprising the amino acid sequence of SEQ ID NO:28, a CDR-H2 comprising the amino acid sequence of SEQ ID NO:29, and a CDR-H3 comprising the amino acid sequence of SEQ ID NO:30; V_{L1} comprises a CDR-L1 comprising the amino acid sequence of SEQ ID NO:46, a CDR-L2 comprising the amino acid sequence of SEQ ID NO: 47, and a CDR-L3 comprising the amino acid sequence of SEQ ID NO:48; V_{H2} comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} comprises a CDR-H1 comprising the amino acid sequence of SEQ ID NO:120, a CDR-H2 comprising the amino acid sequence of SEQ ID NO:121, and a CDR-H3 comprising the amino acid sequence of SEQ ID NO:122; and V_{L3} comprises a CDR-L1 comprising the amino acid sequence of SEQ ID NO:123, a CDR-L2 comprising the amino acid sequence of SEQ ID NO:124, and a CDR-L3 comprising the amino acid sequence of SEQ ID NO:125; or
(j) V_{H1} comprises a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33; V_{L1} comprises a CDR-L1 comprising the amino acid sequence of SEQ ID NO:49, a CDR-L2 comprising the amino acid sequence of SEQ ID NO:50, and a CDR-L3 comprising the amino acid sequence of SEQ ID NO:51; V_{H2} Comprises a CDR-H1 comprising the amino acid sequence of SEQ ID NO:34, a CDR-H2 comprising the amino acid sequence of SEQ ID NO:35, and a CDR-H3 comprising the amino acid sequence of SEQ ID NO:36; V_{L2} comprises a CDR-L1 comprising the amino acid sequence of SEQ ID NO:52, a CDR-L2 comprising the amino acid sequence of SEQ ID NO:53, and a CDR-L3 comprising the amino acid sequence of SEQ ID NO:54; V_{H3} comprises a CDR-H1 comprising the amino acid sequence of SEQ ID NO:120, a CDR-H2 comprising the amino acid sequence of SEQ ID NO:121, and a CDR-H3 comprising the amino acid sequence of SEQ ID NO:122; and V_{L3} comprises a CDR-L1 comprising the amino acid sequence of SEQ ID NO:123, a CDR-L2 comprising the amino acid sequence of SEQ ID NO:124, and a CDR-L3 comprising the amino acid sequence of SEQ ID NO:125.

Item 24: A binding protein comprising four polypeptide chains that form three antigen binding sites, wherein a first polypeptide chain comprises a structure represented by the formula:

V_{L2}-L₁-V_{L1}-L₂-C_{L} [I]

and a second polypeptide chain comprises a structure represented by the formula:

V_{H1}-L₃-V_{H2}-L₄-C_{H1} [II]

and a third polypeptide chain comprises a structure represented by the formula:

V_{H3}-C_{H1} [III]

and a fourth polypeptide chain comprises a structure represented by the formula:

V_{L3}-C_{L} [IV]

wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain; and
L₁, L₂, L₃ and L₄ are amino acid linkers;
wherein the polypeptide of formula I and the polypeptide of formula II form a cross-over light chain-heavy chain pair;
wherein:
(a) V_{L1}, V_{L2} and V_{L3} each independently comprise a variable domain sequence as set forth in any one of SEQ ID NOs:169, 171, and 173; or
(b) V_{L1}, V_{L2} and V_{L3} each independently comprise light chain complementarity determining regions comprising an amino acid sequence selected from the group consisting of SEQ ID NOs: 141-147, 178, and 179;
wherein:
(a) V_{H1}, V_{H2}, and V_{H3} each independently comprise a variable domain sequence as set forth in any one of SEQ ID NOs:168, 170, and 172; or
(b) V_{H1}, V_{H2} and V_{H3} each independently comprise heavy chain complementarity determining regions comprising an amino acid sequence selected from the group consisting of SEQ ID NOs: 129-137.

Item 25: A binding protein comprising four polypeptide chains that form three antigen binding sites, wherein a first polypeptide chain comprises a structure represented by the formula:

V_{L2}-L₁-V_{L1}-L₂-C_{L} [I]

and a second polypeptide chain comprises a structure represented by the formula:

V_{H1}-L₃-V_{H2}-L₄-C_{H1}-hinge-C_{H2}-C_{H3} [II]

and a third polypeptide chain comprises a structure represented by the formula:

V_{H3}-C_{H1}-hinge-C_{H2}-C_{H3} [III]

and a fourth polypeptide chain comprises a structure represented by the formula:

V_{L3}-C_{L} [IV]

wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain;
C_{H2} is an immunoglobulin C_{H2} heavy chain constant domain;
C_{H3} is an immunoglobulin C_{H3} heavy chain constant domain;
hinge is an immunoglobulin hinge region connecting the C_{H1} and C_{H2} domains; and
L₁, L₂, L₃ and L₄ are amino acid linkers;
wherein the polypeptide of formula I and the polypeptide of formula II form a cross-over light chain-heavy chain pair;
wherein:
(a) V_{L1}, V_{L2} and V_{L3} each independently comprise a variable domain sequence as set forth in any one of SEQ ID NOs:169, 171, and 173; or
(b) V_{L1}, V_{L2} and V_{L3} each independently comprise light chain complementarity determining regions comprising an amino acid sequence selected from the group consisting of SEQ ID NOs: 141-147, 178, and 179;
wherein:
(a) V_{H1}, V_{H2}, and V_{H3} each independently comprise a variable domain sequence as set forth in any one of SEQ ID NOs:168, 170, and 172; or
(b) V_{H1}, V_{H2} and V_{H3} each independently comprise heavy chain complementarity determining regions comprising an amino acid sequence selected from the group consisting of SEQ ID NOs: 129-137.

Item 26: The binding protein of item 24, wherein the second and/or the third polypeptide chain further comprises an Fc region linked to C_{H1}, the Fc region comprising an immunoglobulin hinge region and C_{H2} and C_{H3} immunoglobulin heavy chain constant domains.

Item 27: The binding protein of any one of items 24-26, wherein at least one of L₁, L₂, L₃ and L₄, is independently 0 amino acids in length.

Item 28: The binding protein of any one of items 24-26, wherein L₁, L₂, L₃ and L₄ are each independently at least one amino acid in length.

Item 29: The binding protein of any one of items 24-26 and 28, wherein (a) L₁, L₂, L₃ and L₄ each independently are zero amino acids in length or comprise a sequence selected from the group consisting of GGGGSGGGGS (SEQ ID NO:104), GGGGSGGGGSGGGGS (SEQ ID NO:105), S, RT, TKGPS (SEQ ID NO:106), GQPKAAP (SEQ ID NO: 175), and GGSGSSGSGG (SEQ ID NO:148); or (b) L₁, L₂, L₃ and L₄ each independently comprise a sequence selected from the group consisting of GGGGSGGGGS (SEQ ID NO:104), GGGGSGGGGSGGGGS (SEQ ID NO:105), S, RT, TKGPS (SEQ ID NO:106), GQPKAAP (SEQ ID NO: 175), and GGSGSSGSGG (SEQ ID NO:148).

Item 30: The binding protein of any one of items 24-28, wherein:
(a) L₁ comprises the sequence GQPKAAP (SEQ ID NO: 175), L₂ comprises the sequence TKGPS (SEQ ID NO:106), L₃ comprises the sequence S, and L₄ comprises the sequence RT;
(b) L₁ comprises the sequence GGGGSGGGGS (SEQ ID NO:104), L₂ comprises the sequence GGGGSGGGGS (SEQ ID NO:104), L₃ is 0 amino acids in length, and L₄ is 0 amino acids in length;
(c) L₁ comprises the sequence GGSGSSGSGG (SEQ ID NO:148), L₂ comprises the sequence GGSGSSGSGG (SEQ ID NO:148), L₃ is 0 amino acids in length, and L₄ is 0 amino acids in length; or
(d) L₁ comprises the sequence GGGGSGGGGSGGGGS (SEQ ID NO:105), L₂ is 0 amino acids in length, L₃ comprises the sequence GGGGSGGGGSGGGGS (SEQ ID NO:105), and L₄ is 0 amino acids in length.

Item 31: The binding protein of any one of items 24-30, wherein:
(a) V_{H1} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; V_{L1} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144; V_{H2} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; V_{L2} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; V_{H3} comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131; and V_{L3} comprises a CDR-L1 comprising the amino acid sequence of SEQ ID NO:141, a CDR-L2 comprising the amino acid sequence of SEQ ID NO:178, and a CDR-L3 comprising the amino acid sequence of SEQ ID NO:142;
(b) V_{H1} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; V_{L1} Comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; V_{H2} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; V_{L2} comprises a CDR-L1 comprising the amino acid sequence of SEQ ID NO:143, a CDR-L2 comprising the amino acid sequence of SEQ ID NO:47, and a CDR-L3 comprising the amino acid sequence of SEQ ID NO:144; V_{H3} comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131; and V_{L3} comprises a CDR-L1 comprising the amino acid sequence of SEQ ID NO:141, a CDR-L2 comprising the amino acid sequence of SEQ ID NO:178, and a CDR-L3 comprising the amino acid sequence of SEQ ID NO:142;
(c) V_{H1} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; V_{L1} Comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144; V_{H2} comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131; V_{L2} comprises a CDR-L1 comprising the amino acid sequence of SEQ ID NO:141, a CDR-L2 comprising the amino acid sequence of SEQ ID NO:178, and a CDR-L3 comprising the amino acid sequence of SEQ ID NO:142; V_{H3} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; and V_{L3} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147;
(d) V_{H1} comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131; V_{L1} Comprises a CDR-L1 comprising the amino acid sequence of SEQ ID NO:141, a CDR-L2 comprising the amino acid sequence of SEQ ID NO:178, and a CDR-L3 comprising the amino acid sequence of SEQ ID NO:142; V_{H2} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; V_{L2} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144; V_{H3} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; and V_{L3} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147;
(e) V_{H1} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; V_{L1} Comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; V_{H2} comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131; V_{L2} comprises a CDR-L1 comprising the amino acid sequence of SEQ ID NO:141, a CDR-L2 comprising the amino acid sequence of SEQ ID NO:178, and a CDR-L3 comprising the amino acid sequence of SEQ ID NO:142; V_{H3} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; and V_{L3} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144;
(f) V_{H1} comprises a CDR-H1 comprising the amino acid sequence of SEQ ID NO:129, a CDR-H2 comprising the amino acid sequence of SEQ ID NO:130, and a CDR-H3 comprising the amino acid sequence of SEQ ID NO:131; V_{L1} Comprises a CDR-L1 comprising the amino acid sequence of SEQ ID NO:141, a CDR-L2 comprising the amino acid sequence of SEQ ID NO:178, and a CDR-L3 comprising the amino acid sequence of SEQ ID NO:142; V_{H2} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; V_{L2} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; V_{H3} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; and V_{L3} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144;
(g) V_{H1} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; V_{L1} Comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; V_{H2} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; V_{L2} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144; V_{H3} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; and V_{L3} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147;
(h) V_{H1} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; V_{L1} Comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; V_{H2} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; V_{L2} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144; V_{H3} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; and V_{L3} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144;
(i) V_{H1} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; V_{L1} Comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144; V_{H2} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; V_{L2} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; V_{H3} comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; and V_{L3} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; or
(j) V_{H1} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; V_{L1} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144; V_{H2} Comprises a CDR-H1 comprising the amino acid sequence of SEQ ID NO:135, a CDR-H2 comprising the amino acid sequence of SEQ ID NO:136, and a CDR-H3 comprising the amino acid sequence of SEQ ID NO:137; V_{L2} comprises a CDR-L1 comprising the amino acid sequence of SEQ ID NO:145, a CDR-L2 comprising the amino acid sequence of SEQ ID NO:146, and a CDR-L3 comprising the amino acid sequence of SEQ ID NO:147; V_{H3} comprises a CDR-H1 comprising the amino acid sequence of SEQ ID NO:132, a CDR-H2 comprising the amino acid sequence of SEQ ID NO:133, and a CDR-H3 comprising the amino acid sequence of SEQ ID NO:134; and V_{L3} comprises a CDR-L1 comprising the amino acid sequence of SEQ ID NO:143, a CDR-L2 comprising the amino acid sequence of SEQ ID NO:179, and a CDR-L3 comprising the amino acid sequence of SEQ ID NO:144.

Item 32: The binding protein of any one of items 1, 3-16, 18-24, and 26-31, wherein the second polypeptide chain further comprises a first Fc region linked to C_{H1}, the first Fc region comprising an immunoglobulin hinge region and C_{H2} and C_{H3} immunoglobulin heavy chain constant domains, wherein the first Fc region comprises amino acid substitutions at positions corresponding to positions 354 and 366 of human IgG1 or IgG4 according to EU Index, wherein the amino acid substitutions are S354C and T366W; and wherein the third polypeptide chain further comprises a second Fc region linked to C_{H1}, the second Fc region comprising an immunoglobulin hinge region and C_{H2} and C_{H3} immunoglobulin heavy chain constant domains, wherein the second Fc region comprises amino acid substitutions at positions corresponding to positions 349, 366, 368, and 407 of human IgG1 or IgG4 according to EU Index, wherein the amino acid substitutions are Y349C, T366S, L368A, and Y407V.

Item 33: The binding protein of any one of items 1, 3-16, 18-24, and 26-31, wherein the second polypeptide chain further comprises a first Fc region linked to C_{H1}, the first Fc region comprising an immunoglobulin hinge region and C_{H2} and C_{H3} immunoglobulin heavy chain constant domains, wherein the first Fc region comprises amino acid substitutions at positions corresponding to positions 349, 366, 368, and 407 of human IgG1 or IgG4 according to EU Index, wherein the amino acid substitutions are Y349C, T366S, L368A, and Y407V; and wherein the third polypeptide chain further comprises a second Fc region linked to C_{H1}, the second Fc region comprising an immunoglobulin hinge region and C_{H2} and C_{H3} immunoglobulin heavy chain constant domains, wherein the second Fc region comprises amino acid substitutions at positions corresponding to positions 354 and 366 of human IgG1 or IgG4 according to EU Index, wherein the amino acid substitutions are S354C and T366W.

Item 34: The binding protein of any one of items 1, 3-16, 18-24, and 26-33, wherein the second polypeptide chain further comprises a first Fc region linked to C_{H1}, the first Fc region comprising an immunoglobulin hinge region and C_{H2} and C_{H3} immunoglobulin heavy chain constant domains, and wherein the third polypeptide chain further comprises a second Fc region linked to C_{H1}, the second Fc region comprising an immunoglobulin hinge region and C_{H2} and C_{H3} immunoglobulin heavy chain constant domains; wherein the first and second Fc regions comprise amino acid substitutions at positions corresponding to positions 428 and 434 of human IgG1 or IgG4 according to EU Index, wherein the amino acid substitutions are M428L and N434S.

Item 35: The binding protein of any one of items 2, 4-15, 17, 19-23, 25, and 27-31, wherein the C_{H3} domain of the second polypeptide chain comprises amino acid substitutions at positions corresponding to positions 354 and 366 of human IgG1 or IgG4 according to EU Index, wherein the amino acid substitutions are S354C and T366W; and wherein the C_{H3} domain of the third polypeptide chain comprises amino acid substitutions at positions corresponding to positions 349, 366, 368, and 407 of human IgG1 or IgG4 according to EU Index, wherein the amino acid substitutions are Y349C, T366S, L368A, and Y407V.

Item 36: The binding protein of any one of items 2, 4-15, 17, 19-23, 25, and 27-31, wherein the C_{H3} domain of the second polypeptide chain comprises amino acid substitutions at positions corresponding to positions 349, 366, 368, and 407 of human IgG1 or IgG4 according to EU Index, wherein the amino acid substitutions are Y349C, T366S, L368A, and Y407V; and wherein the C_{H3} domain of the third polypeptide chain comprises amino acid substitutions at positions corresponding to positions 354 and 366 of human IgG1 or IgG4 according to EU Index, wherein the amino acid substitutions are S354C and T366W.

Item 37: The binding protein of any one of items 2, 4-15, 17, 19-23, 25, 27-31, 35, and 36, wherein the C_{H3} domains of the second and the third polypeptide chains both comprise amino acid substitutions at positions corresponding to positions 428 and 434 of human IgG1 or IgG4 according to EU Index, wherein the amino acid substitutions are M428L and N434S

Item 38: The binding protein of any one of items 1, 3-16, 18-24, and 26-34, wherein the second polypeptide chain further comprises a first Fc region linked to C_{H1}, the first Fc region comprising an immunoglobulin hinge region and C_{H2} and C_{H3} immunoglobulin heavy chain constant domains; wherein the third polypeptide chain further comprises a second Fc region linked to C_{H1}, the second Fc region comprising an immunoglobulin hinge region and C_{H2} and C_{H3} immunoglobulin heavy chain constant domains; wherein the first and second Fc regions are human IgG1 or IgG4 Fc regions; and wherein only one of the first and the second Fc regions comprises amino acid substitutions at positions corresponding to positions 435 and 436 of human IgG1 or IgG4 according to EU Index, wherein the amino acid substitutions are H435R and Y436F.

Item 39: The binding protein of any one of items 2, 4-15, 17, 19-23, 25, 27-31, and 35-37, wherein the C_{H3} domains of the second and the third polypeptide chains are human IgG1 or IgG4 C_{H3} domains, and wherein only one of the C_{H3} domains comprises amino acid substitutions at positions corresponding to positions 435 and 436 of human IgG1 or IgG4 according to EU Index, wherein the amino acid substitutions are H435R and Y436F.

Item 40: The binding protein of any one of items 1, 3-16, 18-24, 26-34, and 38, wherein the second polypeptide chain further comprises a first Fc region linked to C_{H1}, the first Fc region comprising an immunoglobulin hinge region and C_{H2} and C_{H3} immunoglobulin heavy chain constant domains; wherein the third polypeptide chain further comprises a second Fc region linked to C_{H1}, the second Fc region comprising an immunoglobulin hinge region and C_{H2} and C_{H3} immunoglobulin heavy chain constant domains; wherein the first and second Fc regions are human IgG4 Fc regions; and wherein the first and the second Fc regions each comprise amino acid substitutions at positions corresponding to positions 228 and 409 of human IgG4 according to EU Index, wherein the amino acid substitutions are S228P and R409K.

Item 41: The binding protein of any one of items 2, 4-15, 17, 19-23, 25, 27-31, 35-37, and 39, wherein the C_{H3} domains of the second and the third polypeptide chains are human IgG4 C_{H3} domains, and wherein the C_{H3} domains each comprise amino acid substitutions at positions corresponding to positions 228 and 409 of human IgG4 according to EU Index, wherein the amino acid substitutions are S228P and R409K.

Item 42: The binding protein of any one of items 1, 3-16, 18-24, 26-34, 38, and 40, wherein the second polypeptide chain further comprises a first Fc region linked to C_{H1}, the first Fc region comprising an immunoglobulin hinge region and C_{H2} and C_{H3} immunoglobulin heavy chain constant domains; wherein the third polypeptide chain further comprises a second Fc region linked to C_{H1}, the second Fc region comprising an immunoglobulin hinge region and C_{H2} and C_{H3} immunoglobulin heavy chain constant domains; wherein the first and second Fc regions are human IgG4 Fc regions; and wherein the first and the second Fc regions each comprise amino acid substitutions at positions corresponding to positions 234 and 235 of human IgG4 according to EU Index, wherein the amino acid substitutions are F234A and L235A.

Item 43: The binding protein of any one of items 2, 4-15, 17, 19-23, 25, 27-31, 35-37, 39, and 41, wherein the C_{H3} domains of the second and the third polypeptide chains are human IgG4 C_{H3} domains, and wherein the C_{H3} domains each comprise amino acid substitutions at positions corresponding to positions 234 and 235 of human IgG4 according to EU Index, wherein the amino acid substitutions are F234A and L235A.

Item 44: The binding protein of any one of items 1, 3-16, 18-24, 26-34, 38, and 40, wherein the second polypeptide chain further comprises a first Fc region linked to C_{H1}, the first Fc region comprising an immunoglobulin hinge region and C_{H2} and C_{H3} immunoglobulin heavy chain constant domains; wherein the third polypeptide chain further comprises a second Fc region linked to C_{H1}, the second Fc region comprising an immunoglobulin hinge region and C_{H2} and C_{H3} immunoglobulin heavy chain constant domains; wherein the first and second Fc regions are human IgG1 Fc regions; and wherein the first and the second Fc regions each comprise amino acid substitutions at positions corresponding to positions 234 and 235 of human IgG4 according to EU Index, wherein the amino acid substitutions are L234A and L235A.

Item 45: The binding protein of any one of items 2, 4-15, 17, 19-23, 25, 27-31, 35-37, 39, and 41, wherein the C_{H3} domains of the second and the third polypeptide chains are human IgG1 C_{H3} domains, and wherein the C_{H3} domains each comprise amino acid substitutions at positions corresponding to positions 234 and 235 of human IgG4 according to EU Index, wherein the amino acid substitutions are L234A and L235A.

Item 46: The binding protein of any one of items 2, 4-15, 17, 19-23, 25, 27-31, 35-37, 39, 41, 43, and 45, wherein VH1 and VL1 form a first antigen binding site that specifically binds human CD3, wherein VH2 and VL2 form a second antigen binding site that specifically binds human CD28, and wherein VH3 and VL3 form a third antigen binding site that specifically binds a human tumor target protein.

Item 47: The binding protein of any one of items 2, 4-15, 17, 19-23, 25, 27-31, 35-37, 39, 41, 43, and 45, wherein VH1 and VL1 form a first antigen binding site that specifically binds human CD28, wherein VH2 and VL2 form a second antigen binding site that specifically binds human CD3, and wherein VH3 and VL3 form a third antigen binding site that specifically binds a human tumor target protein.

Item 48: The binding protein of item 46 or item 47, wherein the third antigen binding site specifically binds a human tumor target protein selected from the group consisting of CD19, CD20, CD38, Her2, and LAMP1.

Item 49: The binding protein of any one of items 46-48, wherein the antigen binding site that specifically binds CD3 comprises:
(a) a heavy chain variable domain comprising the amino acid sequence of SEQ ID NO: 152 and a light chain variable domain comprising the amino acid sequence of SEQ ID NO: 153; or
(b) a heavy chain variable domain comprising the amino acid sequence of SEQ ID NO: 154 and a light chain variable domain comprising the amino acid sequence of SEQ ID NO: 155.

Item 50: The binding protein of any one of items 46-49, wherein the antigen binding site that specifically binds CD28 comprises:
(a) a heavy chain variable domain comprising the amino acid sequence of SEQ ID NO: 160 and a light chain variable domain comprising the amino acid sequence of SEQ ID NO: 161; or
(b) a heavy chain variable domain comprising the amino acid sequence of SEQ ID NO: 162 and a light chain variable domain comprising the amino acid sequence of SEQ ID NO: 163.

Item 51: The binding protein of any one of items 46-50, wherein the antigen binding site that specifically binds a tumor target protein comprises:
(a) a heavy chain variable domain comprising the amino acid sequence of SEQ ID NO: 156 and a light chain variable domain comprising the amino acid sequence of SEQ ID NO: 157;
(b) a heavy chain variable domain comprising the amino acid sequence of SEQ ID NO: 158 and a light chain variable domain comprising the amino acid sequence of SEQ ID NO: 159;
(c) a heavy chain variable domain comprising the amino acid sequence of SEQ ID NO: 164 and a light chain variable domain comprising the amino acid sequence of SEQ ID NO: 165;
(d) a heavy chain variable domain comprising the amino acid sequence of SEQ ID NO: 150 and a light chain variable domain comprising the amino acid sequence of SEQ ID NO: 151; or
(e) a heavy chain variable domain comprising the amino acid sequence of SEQ ID NO: 166 and a light chain variable domain comprising the amino acid sequence of SEQ ID NO: 167.

Item 52: The binding protein of any one of items 2, 17, 35-37, 39, 41, 43, and 45, wherein:
(a) VH1 and VL1 form a first antigen binding site that specifically binds human TNFa, VH2 and VL2 form a second antigen binding site that specifically binds human IL13, and VH3 and VL3 form a third antigen binding site that specifically binds human IL4;
(b) VH1 and VL1 form a first antigen binding site that specifically binds human TNFa, VH2 and VL2 form a second antigen binding site that specifically binds human IL4, and VH3 and VL3 form a third antigen binding site that specifically binds human IL13;
(c) VH1 and VL1 form a first antigen binding site that specifically binds human IL4, VH2 and VL2 form a second antigen binding site that specifically binds human TNFa, and VH3 and VL3 form a third antigen binding site that specifically binds human IL13;
(d) VH1 and VL1 form a first antigen binding site that specifically binds human IL4, VH2 and VL2 form a second antigen binding site that specifically binds human IL13, and VH3 and VL3 form a third antigen binding site that specifically binds human TNFa;
(e) VH1 and VL1 form a first antigen binding site that specifically binds human IL13, VH2 and VL2 form a second antigen binding site that specifically binds human IL4, and VH3 and VL3 form a third antigen binding site that specifically binds human TNFa; or
(f) VH1 and VL1 form a first antigen binding site that specifically binds human IL13, VH2 and VL2 form a second antigen binding site that specifically binds human TNFa, and VH3 and VL3 form a third antigen binding site that specifically binds human IL4.

Item 53: The binding protein of item 52, wherein the antigen binding site that specifically binds human TNFa comprises a heavy chain variable domain comprising the amino acid sequence of SEQ ID NO:168 and a light chain variable domain comprising the amino acid sequence of SEQ ID NO:169.

Item 54: The binding protein of item 52 or item 53, wherein the antigen binding site that specifically binds human IL4 comprises a heavy chain variable domain comprising the amino acid sequence of SEQ ID NO:170 and a light chain variable domain comprising the amino acid sequence of SEQ ID NO:171.

Item 55: The binding protein of any one of items 52-54, wherein the antigen binding site that specifically binds human IL13 comprises a heavy chain variable domain comprising the amino acid sequence of SEQ ID NO:172 and a light chain variable domain comprising the amino acid sequence of SEQ ID NO:173.

Item 56: The binding protein of any one of items 1-55, wherein:
(a) the C_{L} domain of the first polypeptide chain is a human kappa C_{L} domain, and the C_{L} domain of the fourth polypeptide chain is a human lambda C_{L} domain; or
(b) the C_{L} domain of the first polypeptide chain is a human lambda C_{L} domain, and the C_{L} domain of the fourth polypeptide chain is a human kappa C_{L} domain.

Item 57: The binding protein of any one of items 2, 17, 25, 35-37, 39, 41, and 43-55, wherein the first polypeptide chain comprises a lambda C_{L} domain; wherein the C_{H3} domain of the second polypeptide chain comprises amino acid substitutions at positions corresponding to positions 354 and 366 of human IgG1 according to EU Index, wherein the amino acid substitutions are S354C and T366W; wherein the C_{H3} domain of the third polypeptide chain comprises amino acid substitutions at positions corresponding to positions 349, 366, 368, 407, 435, and 436 of human IgG1 according to EU Index, wherein the amino acid substitutions are Y349C, T366S, L368A, Y407V, H435R, and Y436F; and wherein the fourth polypeptide chain comprises a kappa C_{L} domain.

Item 58: A binding protein comprising a first polypeptide chain, a second polypeptide chain, a third polypeptide chain and a fourth polypeptide chain wherein:
(a) the first polypeptide chain comprises the amino acid sequence of SEQ ID NO: 4 or an amino acid sequence that is at least 95% identical to the amino acid sequence of SEQ ID NO: 4; the second polypeptide chain comprises the amino acid sequence of SEQ ID NO: 3 or an amino acid sequence that is at least 95% identical to the amino acid sequence of SEQ ID NO: 3; the third polypeptide chain comprises the amino acid sequence of SEQ ID NO: 1 or an amino acid sequence that is at least 95% identical to the amino acid sequence of SEQ ID NO: 1; and the fourth polypeptide chain comprises the amino acid sequence of SEQ ID NO: 2 or an amino acid sequence that is at least 95% identical to the amino acid sequence of SEQ ID NO: 2;
(b) the first polypeptide chain comprises the amino acid sequence of SEQ ID NO: 10 or an amino acid sequence that is at least 95% identical to the amino acid sequence of SEQ ID NO: 10; the second polypeptide chain comprises the amino acid sequence of SEQ ID NO: 9 or an amino acid sequence that is at least 95% identical to the amino acid sequence of SEQ ID NO: 9; the third polypeptide chain comprises the amino acid sequence of SEQ ID NO: 1 or an amino acid sequence that is at least 95% identical to the amino acid sequence of SEQ ID NO: 1; and the fourth polypeptide chain comprises the amino acid sequence of SEQ ID NO: 2 or an amino acid sequence that is at least 95% identical to the amino acid sequence of SEQ ID NO: 2;
(c) the first polypeptide chain comprises the amino acid sequence of SEQ ID NO: 4 or an amino acid sequence that is at least 95% identical to the amino acid sequence of SEQ ID NO: 4; the second polypeptide chain comprises the amino acid sequence of SEQ ID NO: 3 or an amino acid sequence that is at least 95% identical to the amino acid sequence of SEQ ID NO: 3; the third polypeptide chain comprises the amino acid sequence of SEQ ID NO: 13 or an amino acid sequence that is at least 95% identical to the amino acid sequence of SEQ ID NO: 13; and the fourth polypeptide chain comprises the amino acid sequence of SEQ ID NO: 14 or an amino acid sequence that is at least 95% identical to the amino acid sequence of SEQ ID NO: 14;
(d) the first polypeptide chain comprises the amino acid sequence of SEQ ID NO: 10 or an amino acid sequence that is at least 95% identical to the amino acid sequence of SEQ ID NO: 10; the second polypeptide chain comprises the amino acid sequence of SEQ ID NO: 9 or an amino acid sequence that is at least 95% identical to the amino acid sequence of SEQ ID NO: 9; the third polypeptide chain comprises the amino acid sequence of SEQ ID NO: 13 or an amino acid sequence that is at least 95% identical to the amino acid sequence of SEQ ID NO: 13; and the fourth polypeptide chain comprises the amino acid sequence of SEQ ID NO: 14 or an amino acid sequence that is at least 95% identical to the amino acid sequence of SEQ ID NO: 14;
(e) the first polypeptide chain comprises the amino acid sequence of SEQ ID NO: 4 or an amino acid sequence that is at least 95% identical to the amino acid sequence of SEQ ID NO: 4; the second polypeptide chain comprises the amino acid sequence of SEQ ID NO: 3 or an amino acid sequence that is at least 95% identical to the amino acid sequence of SEQ ID NO: 3; the third polypeptide chain comprises the amino acid sequence of SEQ ID NO: 17 or an amino acid sequence that is at least 95% identical to the amino acid sequence of SEQ ID NO: 17; and the fourth polypeptide chain comprises the amino acid sequence of SEQ ID NO: 18 or an amino acid sequence that is at least 95% identical to the amino acid sequence of SEQ ID NO: 18;
(f) the first polypeptide chain comprises the amino acid sequence of SEQ ID NO: 10 or an amino acid sequence that is at least 95% identical to the amino acid sequence of SEQ ID NO: 10; the second polypeptide chain comprises the amino acid sequence of SEQ ID NO: 9 or an amino acid sequence that is at least 95% identical to the amino acid sequence of SEQ ID NO: 9; the third polypeptide chain comprises the amino acid sequence of SEQ ID NO: 17 or an amino acid sequence that is at least 95% identical to the amino acid sequence of SEQ ID NO: 17; and the fourth polypeptide chain comprises the amino acid sequence of SEQ ID NO: 18 or an amino acid sequence that is at least 95% identical to the amino acid sequence of SEQ ID NO: 18;
(g) the first polypeptide chain comprises the amino acid sequence of SEQ ID NO: 4 or an amino acid sequence that is at least 95% identical to the amino acid sequence of SEQ ID NO: 4; the second polypeptide chain comprises the amino acid sequence of SEQ ID NO: 3 or an amino acid sequence that is at least 95% identical to the amino acid sequence of SEQ ID NO: 3; the third polypeptide chain comprises the amino acid sequence of SEQ ID NO: 21 or an amino acid sequence that is at least 95% identical to the amino acid sequence of SEQ ID NO: 21; and the fourth polypeptide chain comprises the amino acid sequence of SEQ ID NO: 22 or an amino acid sequence that is at least 95% identical to the amino acid sequence of SEQ ID NO: 22;
(h) the first polypeptide chain comprises the amino acid sequence of SEQ ID NO: 10 or an amino acid sequence that is at least 95% identical to the amino acid sequence of SEQ ID NO: 10; the second polypeptide chain comprises the amino acid sequence of SEQ ID NO: 9 or an amino acid sequence that is at least 95% identical to the amino acid sequence of SEQ ID NO: 9; the third polypeptide chain comprises the amino acid sequence of SEQ ID NO: 21 or an amino acid sequence that is at least 95% identical to the amino acid sequence of SEQ ID NO: 21; and the fourth polypeptide chain comprises the amino acid sequence of SEQ ID NO: 22 or an amino acid sequence that is at least 95% identical to the amino acid sequence of SEQ ID NO: 22;
(i) the first polypeptide chain comprises the amino acid sequence of SEQ ID NO: 63 or an amino acid sequence that is at least 95% identical to the amino acid sequence of SEQ ID NO: 63; the second polypeptide chain comprises the amino acid sequence of SEQ ID NO: 62 or an amino acid sequence that is at least 95% identical to the amino acid sequence of SEQ ID NO: 62; the third polypeptide chain comprises the amino acid sequence of SEQ ID NO: 60 or an amino acid sequence that is at least 95% identical to the amino acid sequence of SEQ ID NO: 60; and the fourth polypeptide chain comprises the amino acid sequence of SEQ ID NO: 61 or an amino acid sequence that is at least 95% identical to the amino acid sequence of SEQ ID NO: 61;
(j) the first polypeptide chain comprises the amino acid sequence of SEQ ID NO: 69 or an amino acid sequence that is at least 95% identical to the amino acid sequence of SEQ ID NO: 69; the second polypeptide chain comprises the amino acid sequence of SEQ ID NO: 68 or an amino acid sequence that is at least 95% identical to the amino acid sequence of SEQ ID NO: 68; the third polypeptide chain comprises the amino acid sequence of SEQ ID NO: 60 or an amino acid sequence that is at least 95% identical to the amino acid sequence of SEQ ID NO: 60; and the fourth polypeptide chain comprises the amino acid sequence of SEQ ID NO: 61 or an amino acid sequence that is at least 95% identical to the amino acid sequence of SEQ ID NO: 61;
(k) the first polypeptide chain comprises the amino acid sequence of SEQ ID NO: 69 or an amino acid sequence that is at least 95% identical to the amino acid sequence of SEQ ID NO: 69; the second polypeptide chain comprises the amino acid sequence of SEQ ID NO: 68 or an amino acid sequence that is at least 95% identical to the amino acid sequence of SEQ ID NO: 68; the third polypeptide chain comprises the amino acid sequence of SEQ ID NO: 60 or an amino acid sequence that is at least 95% identical to the amino acid sequence of SEQ ID NO: 60; and the fourth polypeptide chain comprises the amino acid sequence of SEQ ID NO: 71 or an amino acid sequence that is at least 95% identical to the amino acid sequence of SEQ ID NO: 71;
(l) the first polypeptide chain comprises the amino acid sequence of SEQ ID NO: 76 or an amino acid sequence that is at least 95% identical to the amino acid sequence of SEQ ID NO: 76; the second polypeptide chain comprises the amino acid sequence of SEQ ID NO: 75 or an amino acid sequence that is at least 95% identical to the amino acid sequence of SEQ ID NO: 75; the third polypeptide chain comprises the amino acid sequence of SEQ ID NO: 73 or an amino acid sequence that is at least 95% identical to the amino acid sequence of SEQ ID NO: 73; and the fourth polypeptide chain comprises the amino acid sequence of SEQ ID NO: 74 or an amino acid sequence that is at least 95% identical to the amino acid sequence of SEQ ID NO: 74;
(m) the first polypeptide chain comprises the amino acid sequence of SEQ ID NO: 82 or an amino acid sequence that is at least 95% identical to the amino acid sequence of SEQ ID NO: 82; the second polypeptide chain comprises the amino acid sequence of SEQ ID NO: 81 or an amino acid sequence that is at least 95% identical to the amino acid sequence of SEQ ID NO:81; the third polypeptide chain comprises the amino acid sequence of SEQ ID NO: 73 or an amino acid sequence that is at least 95% identical to the amino acid sequence of SEQ ID NO: 73; and the fourth polypeptide chain comprises the amino acid sequence of SEQ ID NO: 74 or an amino acid sequence that is at least 95% identical to the amino acid sequence of SEQ ID NO: 74;
(n) the first polypeptide chain comprises the amino acid sequence of SEQ ID NO: 88 or an amino acid sequence that is at least 95% identical to the amino acid sequence of SEQ ID NO:88; the second polypeptide chain comprises the amino acid sequence of SEQ ID NO: 87 or an amino acid sequence that is at least 95% identical to the amino acid sequence of SEQ ID NO: 87; the third polypeptide chain comprises the amino acid sequence of SEQ ID NO: 85 or an amino acid sequence that is at least 95% identical to the amino acid sequence of SEQ ID NO: 85; and the fourth polypeptide chain comprises the amino acid sequence of SEQ ID NO: 86 or an amino acid sequence that is at least 95% identical to the amino acid sequence of SEQ ID NO: 86;
(o) the first polypeptide chain comprises the amino acid sequence of SEQ ID NO: 94 or an amino acid sequence that is at least 95% identical to the amino acid sequence of SEQ ID NO: 94; the second polypeptide chain comprises the amino acid sequence of SEQ ID NO: 93 or an amino acid sequence that is at least 95% identical to the amino acid sequence of SEQ ID NO: 93; the third polypeptide chain comprises the amino acid sequence of SEQ ID NO: 85 or an amino acid sequence that is at least 95% identical to the amino acid sequence of SEQ ID NO: 85; and the fourth polypeptide chain comprises the amino acid sequence of SEQ ID NO: 86 or an amino acid sequence that is at least 95% identical to the amino acid sequence of SEQ ID NO: 86;
(p) the first polypeptide chain comprises the amino acid sequence of SEQ ID NO: 69 or an amino acid sequence that is at least 95% identical to the amino acid sequence of SEQ ID NO: 69; the second polypeptide chain comprises the amino acid sequence of SEQ ID NO: 68 or an amino acid sequence that is at least 95% identical to the amino acid sequence of SEQ ID NO: 68; the third polypeptide chain comprises the amino acid sequence of SEQ ID NO: 73 or an amino acid sequence that is at least 95% identical to the amino acid sequence of SEQ ID NO: 73; and the fourth polypeptide chain comprises the amino acid sequence of SEQ ID NO: 74 or an amino acid sequence that is at least 95% identical to the amino acid sequence of SEQ ID NO: 74;
(q) the first polypeptide chain comprises the amino acid sequence of SEQ ID NO: 69 or an amino acid sequence that is at least 95% identical to the amino acid sequence of SEQ ID NO: 69; the second polypeptide chain comprises the amino acid sequence of SEQ ID NO: 68 or an amino acid sequence that is at least 95% identical to the amino acid sequence of SEQ ID NO: 68; the third polypeptide chain comprises the amino acid sequence of SEQ ID NO: 85 or an amino acid sequence that is at least 95% identical to the amino acid sequence of SEQ ID NO: 85; and the fourth polypeptide chain comprises the amino acid sequence of SEQ ID NO: 86 or an amino acid sequence that is at least 95% identical to the amino acid sequence of SEQ ID NO: 86;
(r) the first polypeptide chain comprises the amino acid sequence of SEQ ID NO: 63 or an amino acid sequence that is at least 95% identical to the amino acid sequence of SEQ ID NO: 63; the second polypeptide chain comprises the amino acid sequence of SEQ ID NO: 62 or an amino acid sequence that is at least 95% identical to the amino acid sequence of SEQ ID NO: 62; the third polypeptide chain comprises the amino acid sequence of SEQ ID NO: 73 or an amino acid sequence that is at least 95% identical to the amino acid sequence of SEQ ID NO: 73; and the fourth polypeptide chain comprises the amino acid sequence of SEQ ID NO: 74 or an amino acid sequence that is at least 95% identical to the amino acid sequence of SEQ ID NO: 74;
(s) the first polypeptide chain comprises the amino acid sequence of SEQ ID NO: 63 or an amino acid sequence that is at least 95% identical to the amino acid sequence of SEQ ID NO: 63; the second polypeptide chain comprises the amino acid sequence of SEQ ID NO: 62 or an amino acid sequence that is at least 95% identical to the amino acid sequence of SEQ ID NO: 62; the third polypeptide chain comprises the amino acid sequence of SEQ ID NO: 85 or an amino acid sequence that is at least 95% identical to the amino acid sequence of SEQ ID NO: 85; and the fourth polypeptide chain comprises the amino acid sequence of SEQ ID NO: 86 or an amino acid sequence that is at least 95% identical to the amino acid sequence of SEQ ID NO: 86;
(t) the first polypeptide chain comprises the amino acid sequence of SEQ ID NO: 4 or an amino acid sequence that is at least 95% identical to the amino acid sequence of SEQ ID NO: 4; the second polypeptide chain comprises the amino acid sequence of SEQ ID NO: 3 or an amino acid sequence that is at least 95% identical to the amino acid sequence of SEQ ID NO: 3; the third polypeptide chain comprises the amino acid sequence of SEQ ID NO: 114 or an amino acid sequence that is at least 95% identical to the amino acid sequence of SEQ ID NO: 114; and the fourth polypeptide chain comprises the amino acid sequence of SEQ ID NO: 115 or an amino acid sequence that is at least 95% identical to the amino acid sequence of SEQ ID NO: 115; or
(u) the first polypeptide chain comprises the amino acid sequence of SEQ ID NO: 10 or an amino acid sequence that is at least 95% identical to the amino acid sequence of SEQ ID NO: 10; the second polypeptide chain comprises the amino acid sequence of SEQ ID NO: 9 or an amino acid sequence that is at least 95% identical to the amino acid sequence of SEQ ID NO: 9; the third polypeptide chain comprises the amino acid sequence of SEQ ID NO: 114 or an amino acid sequence that is at least 95% identical to the amino acid sequence of SEQ ID NO: 114; and the fourth polypeptide chain comprises the amino acid sequence of SEQ ID NO: 115 or an amino acid sequence that is at least 95% identical to the amino acid sequence of SEQ ID NO: 115.

Item 59: An isolated nucleic acid molecule comprising a nucleotide sequence encoding the binding protein of any one of items 1-58.

Item 60: An expression vector comprising the nucleic acid molecule of item 59.

Item 61: An isolated host cell comprising the nucleic acid molecule of item 59.

Item 62: An isolated host cell comprising the expression vector of item 60.

Item 63: The isolated host cell of item 61 or item 62, wherein the host cell is a mammalian cell or an insect cell.

Item 64: A pharmaceutical composition comprising the binding protein of any one of items 1-58 and a pharmaceutically acceptable carrier.

Item 65: A method of preventing and/or treating cancer in a patient comprising administering to the patient a therapeutically effective amount of at least one binding protein of any one of items 1-23 and 32-58 or the pharmaceutical composition of item 64.

Item 66: The method of item 65, wherein the binding protein comprises one antigen binding site that specifically binds a T-cell surface protein and another antigen binding site that specifically binds a tumor target protein.

Item 67: The method of item 66, wherein the binding protein comprises a first antigen binding site that specifically binds CD3, a second antigen binding site that specifically binds CD28, and a third antigen binding site that specifically binds a tumor target protein selected from the group consisting of CD19, CD20, CD38, Her2, and LAMP1.

Item 68: The method of any one of items 65-67, wherein the at least one binding protein is co-administered with a chemotherapeutic agent.

Item 69: A method of preventing and/or treating an inflammatory disease or disorder in a patient comprising administering to the patient a therapeutically effective amount of at least one binding protein of any one of items 1-15, 24-45, and 52-58 or the pharmaceutical composition of item 64.

Item 70: The method of item 69, wherein the binding protein comprises three antigen binding sites that each specifically bind a cytokine target protein selected from the group consisting of IL-4, IL-13 and TNFa.

Item 71: The method of item 69 or item 70, wherein the at least one binding protein is co-administered with an anti-inflammatory agent.

Item 72: The method of any one of items 65-71, wherein the patient is a human.

Item 73: The method of item 65 or item 69, wherein the binding protein is capable of inhibiting the function of one or more target proteins selected from the group consisting of A2AR, APRIL, ATPDase, BAFF, BAFFR, BCMA, BlyS, BTK, BTLA, B7DC, B7H1, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL24, CCL25, CCL26, CCR3, CCR4, CD3, CD19, CD20, CD23, CD24, CD27, CD28, CD38, CD39, CD40, CD70, CD80, CD86, CD122, CD137, CD137L, CD152, CD154, CD160, CD272, CD273, CD274, CD275, CD276, CD278, CD279, CDH1, chitinase, CLEC9, CLEC91, CRTH2, CSF-1, CSF-2, CSF-3, CX3CL1, CXCL12, CXCL13, CXCR3, DNGR-1, ectonucleoside triphosphate diphosphohydrolase 1, EGFR, ENTPD1, FCER1A, FCER1, FLAP, Gi24, GITR, GITRL, GM-CSF, Her2, HHLA2, HMGB1, HVEM, ICOSLG, IDO, IFNα, IgE, IGF1R, IL2Rbeta, IL1, IL1A, IL1B, IL1F10, IL2, IL4, IL4Ra, IL5, IL5R, IL6, IL7, IL7Ra, IL8, IL9, IL9R, IL10, rhIL10, IL12, IL13, IL13Ra1, IL13Ra2, IL15, IL17, IL17Rb, IL18, IL22, IL23, IL25, IL27, IL33, IL35, ITGB4, ITK, KIR, LAG3, LAMP1, leptin, LPFS2, MHC class II, NCR3LG1, NKG2D, NTPDase-1, OX40, OX40L, PD-1H, platelet receptor, S152, SISP1, SLC, SPG64, ST2, Syk kinase, TACI, TDO, T14, TIGIT, TIM3, TLR, TLR2, TLR4, TLR5, TLR9, TNFa, TNFRSF7, Tp55, TREM1, TSLP, TSLPR, TWEAK, VEGF, VISTA, Vstm3, WUCAM, and XCR1.

Item 74: The binding protein of any one of items 1-23 and 32-58 or the composition of item 64 for use in preventing and/or treating cancer in a patient.

Item 75: The binding protein for use or the composition for use of item 74, wherein the binding protein comprises one antigen binding site that specifically binds a T-cell surface protein and another antigen binding site that specifically binds a tumor target protein.

Item 76: The binding protein for use or the composition for use of item 75, wherein the binding protein comprises a first antigen binding site that specifically binds CD3, a second antigen binding site that specifically binds CD28, and a third antigen binding site that specifically binds a tumor target protein selected from the group consisting of CD19, CD20, CD38, Her2, and LAMP1.

Item 77: The binding protein for use or the composition for use of any one of items 74-76, wherein the binding protein is co-administered with a chemotherapeutic agent.

Item 78: The binding protein of any one of items 1-15, 24-45, and 52-58 or the pharmaceutical composition of item 64 for use in preventing and/or treating an inflammatory disease or disorder in a patient.

Item 79: The binding protein for use or the composition for use of item 78, wherein the binding protein comprises three antigen binding sites that each specifically bind a cytokine target protein selected from the group consisting of IL-4, IL-13 and TNFa.

Item 80: The binding protein for use or the composition for use of item 78 or item 79, wherein the binding protein is co-administered with an anti-inflammatory agent.

Item 81: The binding protein for use or the composition for use of any one of items 74-80, wherein the patient is a human.

Item 82: The binding protein for use or the composition for use of item 74 or item 78, wherein the binding protein is capable of inhibiting the function of one or more target proteins selected from the group consisting of A2AR, APRIL, ATPDase, BAFF, BAFFR, BCMA, BlyS, BTK, BTLA, B7DC, B7H1, B7H4, B7H5, B7H6, B7H7, B7RP1, B7-4, C3, C5, CCL2, CCL3, CCL4, CCL5, CCL7, CCL8, CCL11, CCL15, CCL17, CCL19, CCL20, CCL21, CCL24, CCL25, CCL26, CCR3, CCR4, CD3, CD19, CD20, CD23, CD24, CD27, CD28, CD38, CD39, CD40, CD70, CD80, CD86, CD122, CD137, CD137L, CD152, CD154, CD160, CD272, CD273, CD274, CD275, CD276, CD278, CD279, CDH1, chitinase, CLEC9, CLEC91, CRTH2, CSF-1, CSF-2, CSF-3, CX3CL1, CXCL12, CXCL13, CXCR3, DNGR-1, ectonucleoside triphosphate diphosphohydrolase 1, EGFR, ENTPD1, FCER1A, FCER1, FLAP, FOLH1, Gi24, GITR, GITRL, GM-CSF, Her2, HHLA2, HMGB1, HVEM, ICOSLG, IDO, IFNα, IgE, IGF1R, IL2Rbeta, IL1, IL1A, IL1B, IL1F10, IL2, IL4, IL4Ra, IL5, IL5R, IL6, IL7, IL7Ra, IL8, IL9, IL9R, IL10, rhIL10, IL12, IL13, IL13Ra1, IL13Ra2, IL15, IL17, IL17Rb, IL18, IL22, IL23, IL25, IL27, IL33, IL35, ITGB4, ITK, KIR, LAG3, LAMP1, leptin, LPFS2, MHC class II, NCR3LG1, NKG2D, NTPDase-1, OX40, OX40L, PD-1H, platelet receptor, PROM1, S152, SISP1, SLC, SPG64, ST2, STEAP2, Syk kinase, TACI, TDO, T14, TIGIT, TIM3, TLR, TLR2, TLR4, TLR5, TLR9, TMEF1, TNFa, TNFRSF7, Tp55, TREM1, TSLP, TSLPR, TWEAK, VEGF, VISTA, Vstm3, WUCAM, and XCR1.

Item 83: A method of purifying a binding protein produced by a host cell, comprising:
(a) producing the binding protein of any one of items 2, 17, 25, 35-37, 41, 43, 45, and 46-55 in a host cell, wherein only one of the C_{H3} domain of the second polypeptide chain and the C_{H3} domain of the third polypeptide chain comprises amino acid substitutions at positions corresponding to positions 435 and 436 of human IgG1 or IgG4 according to EU Index, wherein the amino acid substitutions are H435R and Y436F;
(b) contacting the binding protein produced in (a) with Protein A; and
(c) eluting the binding protein from Protein A under conditions suitable for isolating the binding protein away from binding proteins comprising either 0 or 2 C_{H3} domains comprising the amino acid substitutions are H435R and Y436F, thereby purifying the binding protein.

Item 84: The method of item 83, wherein the C_{L} domain of the first polypeptide chain is a human kappa C_{L} domain, and the C_{L} domain of the fourth polypeptide chain is a human lambda C_{L} domain; or the C_{L} domain of the first polypeptide chain is a human lambda C_{L} domain, and the C_{L} domain of the fourth polypeptide chain is a human kappa C_{L} domain, and the method further comprises:
(d) contacting the binding protein eluted in (c) with a kappa light chain affinity medium; and
(e) eluting the binding protein from the kappa light chain affinity medium under conditions suitable for isolating the binding protein away from binding proteins comprising only lambda C_{L} domains.

Item 85: The method of item 84, further comprising, after (e):
(f) contacting the binding protein eluted in (e) with a lambda light chain affinity medium; and
(g) eluting the binding protein from the lambda light chain affinity medium under conditions suitable for isolating the binding protein away from binding proteins comprising only kappa C_{L} domains.

Item 86: The method of item 83, wherein the C_{L} domain of the first polypeptide chain is a human kappa C_{L} domain, and the C_{L} domain of the fourth polypeptide chain is a human lambda C_{L} domain; or the C_{L} domain of the first polypeptide chain is a human lambda C_{L} domain, and the C_{L} domain of the fourth polypeptide chain is a human kappa C_{L} domain, and the method further comprises:
(d) contacting the binding protein eluted in (c) with a lambda light chain affinity medium; and
(e) eluting the binding protein from the lambda light chain affinity medium under conditions suitable for isolating the binding protein away from binding proteins comprising only kappa C_{L} domains.

Item 87: The method of item 86, further comprising, after (e):
(f) contacting the binding protein eluted in (e) with a kappa light chain affinity medium; and
(g) eluting the binding protein from the kappa light chain affinity medium under conditions suitable for isolating the binding protein away from binding proteins comprising only lambda C_{L} domains.

Item 88: The method of any one of items 83-87, wherein the first polypeptide chain comprises a lambda C_{L} domain; wherein the C_{H3} domain of the second polypeptide chain comprises amino acid substitutions at positions corresponding to positions 354 and 366 of human IgG1 according to EU Index, wherein the amino acid substitutions are S354C and T366W; wherein the C_{H3} domain of the third polypeptide chain comprises amino acid substitutions at positions corresponding to positions 349, 366, 368, 407, 435, and 436 of human IgG1 according to EU Index, wherein the amino acid substitutions are Y349C, T366S, L368A, Y407V, H435R, and Y436F; and wherein the fourth polypeptide chain comprises a kappa C_{L} domain.

Item 89: The method of any one of items 83-88, wherein the binding protein is detected in one or more of (c) and (e) using hydrophobic interaction chromatography (HIC).

Item 90: The method of any one of items 83-89, wherein the C_{H3} domains of the second and the third polypeptide chains are human IgG1 or IgG4 C_{H3} domains.

### EXAMPLES

The Examples that follow are illustrative of specific embodiments of the disclosure, and various uses thereof. They are set forth for explanatory purposes only, and should not be construed as limiting the scope of the invention in any way.

### Example 1: Materials and Methods

The following materials and methods were used for the experiments described in Examples 2-5.

### Trispecific Antibody Design

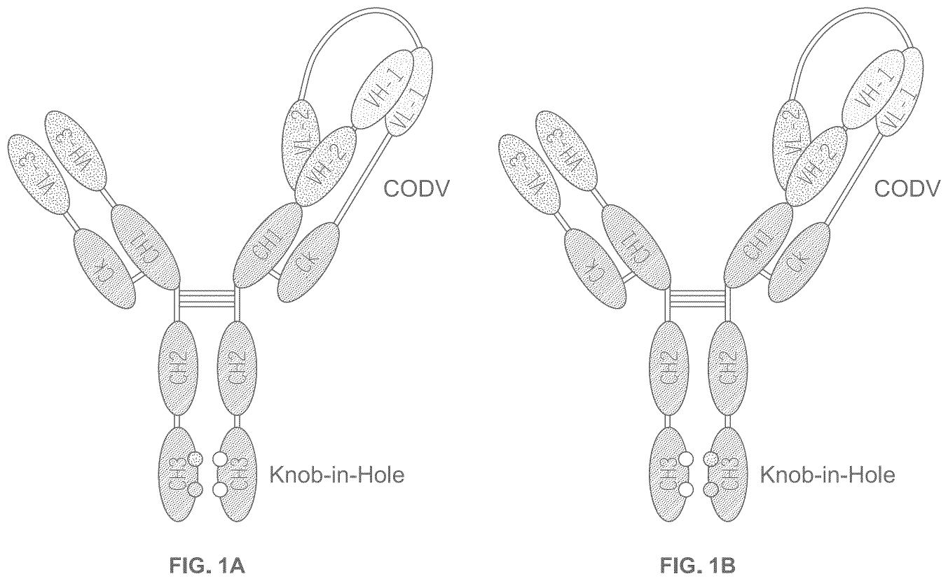

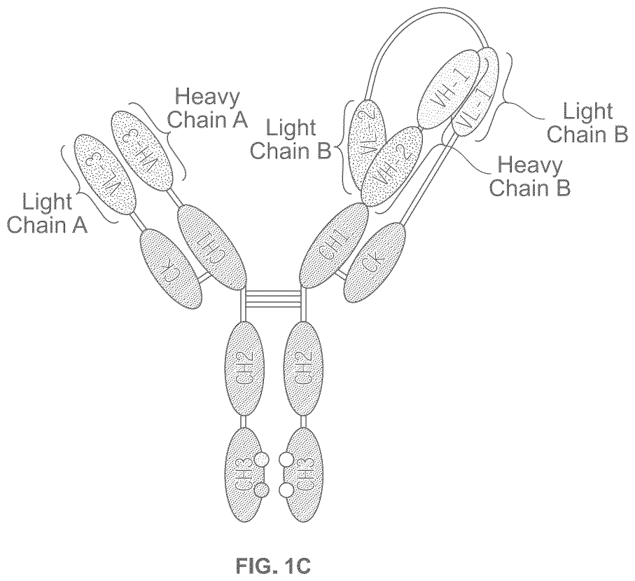

A schematic illustration of the general trispecific antibody design is illustrated in FIGS. 1A-1C. Individual trispecific antibodies were designed based on 5 parameters: 1) Selection of antibody binding sites; 2) Consideration of the position of each binding site; 3) Choice of linkers for the bispecific binding arm (i.e., heavy chain/light chain B in FIG. 1C); 4). “Knob” and “Hole” mutation integration into respective halves of the antibody; 5) Choice of Fc isotype (IgG1 or IgG4). After assembly of the amino acid sequences for each trispecific molecule, four genes for each trispecific Ab were synthesized using human preferred codons (CambrY Applied Biosciences, Cambridge, MA, USA), and cloned into a eukaryotic expression vector.

### Production and Purification of Trispecific Antibodies

Trispecific antibodies were produced by transient transfection of 4 expression plasmids into Expi293 cells using ExpiFectamine™ 293 Transfection Kit (Thermo Fisher Scientific) according to manufacturer's protocol. Briefly, 25% (w/w) of each plasmid was diluted into Opti-MEM, mixed with pre-diluted ExpiFectamine reagent for 20-30 minutes at room temperature (RT), and added into Expi293 cells (2.5×10⁶ cells/ml). An optimization of transfection to determine the best ratio of plasmids was often used in order to produce the trispecific antibody with good yield and purity.

4-5 days post transfection, the supernatant from transfected cells was collected and filtered through 0.45 μm filter unit (Nalgene). The trispecific antibody in the supernatant was purified using a 3-step procedure. First, protein A affinity purification was used, and the bound Ab was eluted using “IgG Elution Buffer” (Thermo Fisher Scientific). Second, product was dialyzed against PBS (pH7.4) overnight with 2 changes of PBS buffer. Any precipitate was cleared by filtration through 0.45 μm filter unit (Nalgene) before next step. Third, size-exclusion chromatography (SEC) purification (Hiload 16/600 Superdex 200 pg, or Hiload 26/600 Superdex 200 pg, GE Healthcare) was used to remove aggregates and different species in the prep. The fractions were analyzed on reduced and non-reduced SDS-PAGE to identify the fractions that contained the monomeric trispecific antibody before combining them. The purified antibody can be aliquoted and stored at −80° C. long term.

### ELISA Assays

The binding properties of the purified antibodies were analyzed either using ELISA or SPR methods. For ELISA, corresponding antigens for each binding site in the trispecific antibody were used to coat a 96-well Immuno Plate (Nunc 439454, Thermo Fisher Scientific) overnight at 4° C. using 2 μg/ml each antigen in PBS (pH7.4). The coated plate was blocked using 5% skim milk+2% BSA in PBS for one hour at RT, followed by washing with PBS+0.25% Tween 20 three times (Aqua Max 400, Molecular Devices). Serial dilution of antibodies (trispecific and control Abs) were prepared and added onto the ELISA plates (100 μl/well in duplicate), incubated at RT for one hour, followed by washing 5 times with PBS+0.25% Tween 20.

After washing, the HRP conjugated secondary anti-human Fab (1:5000, Cat. No. 109-035-097, Jackson ImmunoResearch Inc) was added to each well and incubated at RT for 30 minutes. After washing 5 times with PBS+0.25% Tween 20, 100 μl of TMB Microwell Peroxidase Substrate (KPL, Gaithersburg, MD, USA) was added to each well. The reaction was terminated by adding 50 μl 1M H₂SO₄, and OD₄₅₀ was measured using SpectraMax M5 (Molecular Devices) and analyzed using SoftMax Pro6.3 software (Molecular Devices). The final data was transferred to GraphPad Prism software (GraphPad Software, CA, USA), and plotted as shown. EC50 was calculated using the same software.

### SPR Assays

Two pairs of heavy and light chains were selected for full kinetic analysis. Kinetic characterization of purified antibodies was performed using surface plasmon resonance (SPR) technology on a BIACORE 3000 (GE Healthcare). A capture assay using a tag specific antibody capture and orientation of the investigated antibodies was used. For capture of Fc containing protein constructs the human antibody capture kit (GE Healthcare) was used, for capture of His tag containing protein constructs the His capture kit (GE Healthcare) was used. The capture antibody was immobilized via primary amine groups (11000 RU) on a research grade CM5 chip (GE Life Sciences) using standard procedures. The analyzed antibody was captured at a flow rate of 10 μL/min with an adjusted RU value that would result in maximal analyte binding signal of typically 30 RU.

For an exemplary assay, recombinant human IL13 (catalog #IL012) and human IL4 (catalog #IL004) were purchased from Millipore, recombinant human TNFa (catalog #H8916) was purchased from Sigma Aldrich. Binding kinetics were measured against recombinant human IL4 and IL13 over a concentration range between 0.1 to 3 nM for IL4 and 0.8 to 25 nM for IL13. For human TNFα a concentration range from 3 to 100 nM was used. As assay buffer HBS EP (10 mM HEPES, pH 7.4, 150 mM NaCl, 3 mM EDTA, and 0.005% Surfactant P20) was used at a flow rate of 30 μl/min. Chip surfaces were regenerated with the regeneration solution of the respective capture kit. Kinetic parameters were analyzed and calculated in the BIAevaluation program package v4.1 using a flow cell without captured antibody as reference and the 1:1 Langmuir binding model with mass transfer. To study simultaneous binding of antigens the trispecific antibodies were captured by an anti-human antibody capture surface. Antigens were used in single concentrations with IL4 at 3 nM, IL13 at 25 nM and TNFα at 100 nM. To show simultaneous binding of all three antigens, a mixture of IL4, IL13 and TNFα was injected. In separate analysis cycles, IL13 was injected alone, followed by either IL4 or TNFα, and followed by a co-inject of either IL4/TNFα or a co-inject of TNFα/IL4. The final response measured in each cycle was compared to show similarity of consecutive binding of either two or three antigens and simultaneous binding of a mixture of all three antigens.

### In Vitro T Cell Activation and Proliferation Assays

Human PBMCs were purified from buffy coat purchased from Blood Research Component (Brookline, MA, USA) using Ficoll-Paque Plus method. Briefly, fresh buffy coat was first diluted at 1:3 ratio in PBS (pH7.4), and mixed with Ficoll-Paque Plus solution (Ficoll) thoroughly before use by inverting the bottle several times. 15 mL density gradient medium was added to each Leucosep® tube and spin for 30s at 1000×g, RT. The medium is now located below the porous barrier. 30-40 mL diluted buffy coat then was carefully poured into each Leucosep tube, and centrifuged at 800×g for 15 minutes at room temperature, with the brake off and Max accel at 5. Plasma layer was removed, and the rest of the supernatant, which contains the enriched PBMCs, was transferred into a new tube (Leucosep tube was not held in the inverted position for longer than 2 seconds). Enriched PBMCs were washed with 45 ml PBS, and spun down at 250×g for 10 minutes at room temperature. Wash was repeated, and multiple tubes were combined into one tube. Cells were resuspended in 20 mL PBS and counted using a Bio-Rad TC20.

To set up the in vitro T cell activation assay, purified human PBMCs were resuspended in culture medium (RPMI1640 with 10% FBS and supplemented with glutamine/Streptomycin)(Thermo Fisher Scientific) (10⁶ cells/ml). Indicated concentrations of different trispecific and control antibodies were added to each well, or used to coat the plate before use as described in Stebbings, R. et al. (2007) J. Immunol. 179:3325-3331, and incubated for 16-24 hours in a tissue culture incubator. The cells were spin down, and the supernatant was either collected for measuring cytokine release, or discarded. The cells were stained with florescent labeled antibodies for T cell markers (CD3, CD4, CD8, etc.) and activation markers (CD69, CD62L, etc.), and analyzed by running the samples on an Fortessa flow cytometer (Beckton Dickinson, San Jose, CA), followed by analysis using the Flowjo software (FlowJo v10) and plotted as shown.

To set up the in vitro T cell proliferation assay, purified human PBMCs were resuspended in culture medium (RPMI1640 with 10% FBS and supplemented with glutamine/Streptomycin)(Thermo Fisher Scientific) (10⁶ cells/ml). Indicated concentrations of different trispecific and control antibodies were added to each well and incubated for 1-7 days in a tissue culture incubator. The cells were spun down, and the supernatant was either collected for measuring cytokine release, or discarded. The cells were stained with florescent labeled antibodies for T cell markers (CD3, CD4, CD8, etc.) and activation markers (CD69, CD62L, etc.), and analyzed by running the samples on an Fortessa flow cytometer (Beckton Dickinson, San Jose, CA), followed by analysis using the Flowjo software (FlowJo v10) and plotted as shown.

### In Vitro Cell Killing Assay

Purified human PBMCs were using for in vitro killing assays against various cancer cells using different trispecific antibodies. Briefly, the killing assay was set up in 96-well V-bottom plate. For each plate, 40 ml PBMCs from each donor were plated at 2×10\hat{ }6 cells/ml, and 30 ml of PKH26 (Sigma #MINI26) labeled target cells at 2.5×10\hat{ }5 cells/ml (4 μL of dye to stain up to 1×10\hat{ }7 cells) were prepared. First 20 μL/well test proteins at various concentrations or PMA were added into each well, followed by adding 80 μL/well labeled target cells into each well (2×10\hat{ }4 cells/well). 100 μL of PBMC were then added to each well, reaching E:T=10:1 well (2×10\hat{ }5 cells/well), and incubated for 24 hours at 37° C. 5% CO2 incubator. The cells were spin down, and the supernatant was either collected for measuring cytokine release, or discarded. The cells were stained with Vivid LIVE/DEAD™ Fixable Violet Dead Cell Staining buffer (Life Technology #L34955) (staining buffer was prepared by adding 60 μL Vivid reagent into 60 ml PBS). Cells were resuspended into 100 μL staining buffer by incubation for 15 min at RT in the dark. After washing the cells with 1×PBS, the cells were resuspended in 200 μL PBS with 0.5% Paraformaldehyde, and PKH26+Vivid+ cancer cells were collected by Fortessa flow cytometer (Beckton Dickinson, San Jose, CA), followed by analysis using the Flowjo software. The percentage of killing is calculated as “specific killing-spontaneous killing/total cells and plotted as shown.

### Cytokine Release Assay

For measuring inflammatory cytokine concentrations in the in vitro activation assays, in vitro killing assays, in vivo activation assays in CD34+ umbilical cord cell humanized NSG mice, and the toxicity study, cell culture supernatant was collected, and serum samples were diluted according to manufacturer's protocol using Milliplex Human High Sensitivity T cell I3-plex Kit (EMD Millipore). These were subsequently analyzed by EMD Millipore MAGPIX® System, and MILLIPLEX® Analyst 5.1 software.

### In Vivo Mouse Models and Efficacy Studies

Human CD34+ hematopoietic stem cell-engrafted NSG mice (hu-CD34) were used as an in vivo mouse model. These mice develop multi-lineage human immune cells, and are a validated platform for immuno-oncology efficacy studies (see, e.g., Shultz, L. D. et al. (2014) Cold Spring Harb. Protoc. 2014:694-708). Hu-CD34⁺ NSG mice are produced by injecting CD34⁺ hematopoietic stem cells, showing effective multi-lineage engraftment of human immune cell populations including T cells, B cells and some other populations (McDermott, S. P. et al. (2010) Blood 116:193-200). Multi-lineage hematopoiesis occurs within 12 weeks. Engraftment is stable for over one year without graft-versus-host disease.

For the efficacy study using hu-CD34 NSG mice, mice were purchased from The Jackson Laboratory (Maine, USA), and human cell populations were validated before use. In general, 5×10⁶ tumor cells mixed in Matrigel (BD Biosciences) (50% v/v) were used for inoculating tumor in each mouse. Once tumor size reached the range of 100-150 mm³, mice were selected and randomized into each group for study. Antibodies were given intravenously at given doses 3 times weekly. Body weight was monitored 1-3 times weekly. Tumor size was measured by caliper tumor measurements 1-3 times/week. All mice were terminated when the tumor size reached 1,500 mm³, or 24 hours after the last dose. Terminal blood samples (0.3 mL) were collected into serum separator tubes, mixed by gently inverting five times, and placed into a tube rack. Terminal tumors were also collected and weighed before being put into fixative for immunohistochemistry analysis.

Human PBMC humanized (hu-PBMC) NSG mice were used as another in vivo mouse model. These mice are produced by injecting purified human PBMC from health donors, which have the fastest engraftment rate using adult peripheral blood mononuclear cells and enable short-term studies requiring a strong effector and memory T cell and NK cell function, and are suitable for short term efficacy study (3-4 weeks) due to graft-versus-host disease.

For the efficacy study using hu-PBMC NSG mice, 8-10 week old NSG mice (Cat. No: 005557, NOD.Cg-Prkdcscid Il2rgtm1Wjl/SzJ) were purchased from The Jackson Laboratory (Maine, USA). Each mouse was innoculated with 5×10⁶ tumor cells mixed in Matrigel (BD Biosciences) (50% v/v). Once tumor size reached the range of 50-100 mm³, 10×10⁶ human PBMCs from a healthy donor were reconstituted to each mouse. Human cell reconstitution was validated the next day. Once tumor size reached the range of 100-150 mm³, mice were selected and randomized into each group for study. Antibodies were given intravenously at given doses 3 times weekly. Body weight was monitored 1-3 times weekly. Tumor size was measured by caliper tumor measurements 1-3 times/week. All mice were terminated when the tumor size reached 1,500 mm³ or 24 hours after the last dose. Terminal blood samples (0.3 mL) were collected into serum separator tubes, mixed by gently inverting five times, and placed into a tube rack. Terminal tumors were also collected and weighed before being put into fixative for immunohistochemistry analysis.

### NHP Tolerability and Pharmacokinetic Study

All NHP studies were carried out by Covance (Princeton, New Jersey, USA) according to Covance ICUCA protocol. Drug- and protein-naïve or protein-naïve male Cynomolgus Monkeys were used in all studies. Based on study design, monkeys were selected and grouped for each trispecific antibody. Antibody was given by intravenous infusion for 1 hour via saphenous vein. Increasing doses were given on consecutive days for low doses (<10 μg/kg), but with a 1-2 day interval for higher doses (>10 μg/kg) for observation purposes. Blood samples were collected at 0 hour (Day 1 only), 0.5 hour (mid-infusion), 1, and 6 hours from start of infusion for all animals after each dose, as specified. Additional unscheduled blood samples were collected at the discretion of the study director, pathologist, and/or clinical veterinarian. All animals were returned to colony on Day 60. PBMC and serum from the blood samples were prepared using standard methods, and preserved for future analysis.

### Luciferase Reporter Assay

GloResponse™ IL2-luc2P Jurkat Cells, Thaw and Use (Promega part #CS187002) and GloResponse™ NFAT-Luc2 Jurkat Cells (Promega Cat #CS176401) were purchased from Promega (WI, USA), and prepared for use according to manufacturer's protocol.

Briefly, the cells were thawed for 2 min in a 37° C. water bath and gently transferred to a 15 mL conical centrifuge tube containing 10 mL pre-warmed R10 media. Tube was centrifuged at 300 g for 5 min at RT. Supernatant was removed, and the cells were resuspended in 20 mL pre-warmed R10 media and transferred to a 75 cm2 culture flask, followed by incubation in 37° C. tissue culture incubator until cells were growing and stable (˜3-4 days). The cells were split twice a week to 0.1e6 cells/mL. Cells were maintained in R10+Hygromycin B media for selection. Cells were used for assays ˜7 days after thawing.

For antibody stimulation, trispecific or control antibodies were prepared at various concentrations and serially diluted in PBS. 25 μL of antibodies were dispensed per well. For plate-bound Abs, Maxisorp plate was used and incubated at 4° C. overnight. For soluble Abs, a U-bottom plate was used. Reporter cells were resuspended to 0.3-0.5 e6/mL, and 175 uL cells were added to each well, and incubated in 37° C. CO₂ incubator for 6 hours. The plate was then taken out of the incubator and allowed to equilibrate to ambient temperature (10-15 min). Then 50 μl of Bio-Glo™ Reagent (Promega Cat #G7941) (ambient temperature) was added to the each well of the assay plate. After incubation for 5 minutes, luminescence activity was measured using MicroBeta2 LumiJET microplate counter (Perkin Elmer; is read time). Data were plotted using GraphPad Prism software.

### Conformational Stability

Thermostability measurements (e.g., melting points, Tm) were determined using differential scanning fluorimetry (DSF). Samples were diluted in D-PBS buffer (Invitrogen) to a final concentration of 0.2 μg/μl including a 4× concentrated solution of SYPRO-Orange dye (Invitrogen, 5000× stock in DMSO) in D-PBS in white semi-skirt 96-well plates (BIORAD). All measurements were done in duplicate using a MyiQ2 real time PCR instrument (BIORAD). Negative first derivative curves (−d(RFU)/dT) of the melting curves were generated in the iQ5 Software v2.1 (BIORAD). Data were then exported into Microsoft Excel for Tm determination and graphical display of the data.

### IC50 Measurements

Detection of Antibody Activity Against IL-4 and IL-13 with a Reporter Cell Line

Activities of bispecific antibodies or derivatives against cytokines IL4 and IL13 were determined in commercially available HEK-Blue IL-4/IL-13 reporter cells (InvivoGen). HEK-Blue IL-4/IL-13 cells are designed to monitor the activation of the STAT6 pathway by IL-4 or IL13. Stimulation of the cells with either cytokine results in production of the reporter gene secreted embryonic alkaline phosphatase (SEAP) which can be measured in the culture supernatant with the QUANTI-Blue assay. To test antibody activities against IL4 or IL13, the cytokines were pre-incubated for 1 hour with different concentrations of the antibodies and added to 50.000 HEK-Blue IL-4/IL-13 cells. Cytokine-mediated induction of SEAP was measured after 24 hours incubation in the cell culture supernant with the QUANTI-Blue assay (InvivoGen). Each experiment was performed with n=3 datapoints for each antibody concentration. The half-maximal inhibitory concentration (IC50) for each antibody was calculated via the internal application Biostat-Speed V2.0 (Sanofi).

Detection of Antibody Activity Against TNFα with a Reporter Cell Line

Activities of bispecific antibodies or derivatives against TNFa were determined by using commercially available HEK-Blue TNF-a reporter cells (InvivoGen). HEK-Blue TNF-a cells are designed to detect bioactive TNFa by monitoring the activation of the NFkB pathway via the expression of the reporter gene secreted embryonic alkaline phosphatase (SEAP) which can be measured in the culture supernatant with an QUANTI Blue Assay (InvivoGen). To determine antibody activities against TNFα the cytokines were pre-incubated for 1 hour with different concentrations of the antibodies and added to 50,000 HEK Blue TNF-a cells. Cytokine mediated induction of SEAP was measured after 24 hours in the culture supernatant with the QUANTI-Blue assay (InvivoGen). Each experiment was performed with n=3 datapoints for each antibody concentration. The half maximal inhibitory concentration for each antibody was calculated.

### Example 2: Overview of the Trispecific Binding Proteins

A novel strategy was developed for the generation of trispecific binding proteins. The trispecific proteins comprised four polypeptides that formed three target binding sites (FIGS. 1A-C). Each target binding site comprised the V_{H} and V_{L} domain from an antibody that targeted a distinct human antigen target (See e.g., Table 1). The trispecific binding proteins contained a first pair of polypeptides that possessed dual variable domains having a cross-over orientation forming two distinct antigen binding sites (called the CODV Ig format), and a second pair of polypeptides, each with a single variable domain that formed a third antigen binding site (FIGS. 1A and 1B).

TABLE 1
Heavy and light chain SEQ ID NOs for
binding proteins 1-21, and the target
antigens to which the binding proteins are directed.

| Binding | | |
|---|---|---|
| Protein # | SEQ ID NOS | Directed to: |
| 1 | 1, 2, 3, 4 | Her2 × (CD28 × CD3) |
| 2 | 1, 2, 9, 10 | Her2 × (CD28 × CD3) |
| 3 | 13, 14, 3, 4 | CD19 × (CD28 × CD3) |
| 4 | 13, 14, 9, 10 | CD19 × (CD28 × CD3) |
| 5 | 17, 18, 3, 4 | CD38 × (CD28 × CD3) |
| 6 | 17, 18, 9, 10 | CD38 × (CD28 × CD3) |
| 7 | 21, 22, 3, 4 | LAMP1 × (CD28 × CD3) |
| 8 | 21, 22, 9, 10 | LAMP1 × (CD28 × CD3) |
| 9 | 60, 61, 62, 63 | TNFa × (IL4 × IL13) |
| 10 | 60, 61, 68, 69 | TNFa × (IL13 × IL4) |
| 11 | 60, 71, 68, 69 | TNFa × (IL13 × IL4) |
| 12 | 73, 74, 75, 76 | IL13 × (IL4 × TNFa) |
| 13 | 73, 74, 81, 82 | IL13 × (TNFa × IL4) |
| 14 | 85, 86, 87, 88 | IL4 × (IL13 × TNFa) |
| 15 | 85, 86, 93, 94 | IL4 × (TNFa × IL13) |
| 16 | 73, 74, 68, 69 | IL13 × (IL13 × IL4) |
| 17 | 85, 86, 68, 69 | IL4 × (IL13 × IL4) |
| 18 | 73, 74, 62, 63 | IL13 × (IL4 × IL13) |
| 19 | 85, 86, 62, 63 | IL4 × (IL4 × IL13) |
| 20 | 114, 115, 3, 4 | CD20 × (CD28 × CD3) |
| 21 | 114, 115, 9, 10 | CD20 × (CD28 × CD3) |

The first pair of polypeptides (that possessed the dual variable domains) comprised a first polypeptide having the structure V_{L2}-Linker-V_{L}-Linker-Immunoglobulin light chain constant domain, and a second polypeptide having the structure V_{H1}-Linker-V_{H2}—Linker-Immunoglobulin C_{H1} heavy chain constant domain, resulting in a pair of polypeptides which had a cross over orientation that formed two distinct antigen binding sites: V_{H1}-V_{L1} and V_{H2}-V_{L2} (FIG. 1C, see light and heavy chains B). Table A provides a summary of the design of the bispecific arm (i.e., the arm comprising heavy and light chains B) of IgG1 and IgG4 variants of representative trispecific binding proteins, including indicating the various combinations of the linkers used in the bispecific arm of the trispecific binding proteins. The second pair of polypeptides (that each possessed a single variable domain) comprised a first polypeptide having the structure V_{H3}-Immunoglobulin C_{H1} heavy chain constant domain, and a second polypeptide having the structure V_{L3}-Immunoglobulin light chain constant domain, resulting in a pair of polypeptides that formed a third antigen binding site: V_{H3}-V_{L3} (FIG. 1C, see light and heavy chains A). Furthermore, the trispecific binding proteins were constructed such that either of the C_{H3} domains could include a knob or a hole modification to facilitate antibody heterodimerization (FIG. 1).

TABLE A
summary of the design of the bispecific arm of the trispecific binding
proteins as an IgG1 (Hole) or IgG4 (Hole)

| | | CD28 × CD3 | | | CD3 × CD28 | | | CD28 × CD3 | | | CD3 × CD28 | | |
|---|---|---|---|---|---|---|---|---|---|---|---|---|---|
| | | HC-1 | HC-2 | HC-3 | HC-1 | HC-2 | HC-3 | HC-1 | HC-2 | HC-3 | HC-1 | HC-2 | HC-3 |
| CD28 × | LC-1 | X | | | | | | | | | | | |
| CD3 | LC-2 | | X | | | | | | | | | | |
| | LC-3 | | | X | | | | | | | | | |
| CD3 × | LC-1 | | | | X | | | | | | | | |
| CD28 | LC-2 | | | | | X | | | | | | | |
| | LC-3 | | | | | | X | | | | | | |
| CD28 × | LC-1 | | | | | | | X | | | | | |
| CD3 | LC-2 | | | | | | | | X | | | | |
| | LC-3 | | | | | | | | | X | | | |
| CD3 × | LC-1 | | | | | | | | | | X | | |
| CD28 | LC-2 | | | | | | | | | | | X | |
| | LC-3 | | | | | | | | | | | | X |

Linkers:
[L3, L4]/[L1, L2]-[S, RT]/[GQPKAAP (SEQ ID NO: 175), TKGPS (SEQ ID NO: 106)]; [,]/[GGGGSGGGGS (SEQ ID NO: 104), GGGGSGGGGS (SEQ ID NO: 104)]; or [GGGGSGGGGSGGGGS (SEQ ID NO: 105),]/[GGGGSGGGGSGGGGS (SEQ ID NO: 105),]

### Example 3: In Vitro Binding Activity and Antibody-Mediated Specific Killing of T Cell Engagers

This example describes in vitro assays for characterizing the activities of the T cell engagers.

Using the approach described in Example 2 above for trispecific binding protein design, four trispecific binding proteins (Binding Proteins 1, 3, 5, and 6) were generated. These trispecific binding proteins were created by grafting onto a trispecific binding protein framework the V_{H} and V_{L} domains isolated from antibodies targeting distinct human proteins: CD3, CD19, CD28, CD38, or Her2. Binding Protein 1 was constructed such that the first pair of polypeptides (which formed two antigen binding sites) targeted CD28 and CD3, and the second pair of polypeptides (which formed the single antigen binding site) targeted Her2 (Binding Protein 1=Her2×(CD28×CD3)). Binding Protein 3 was constructed such that the first pair of polypeptides (which formed two antigen binding sites) targeted CD28 and CD3, and the second pair of polypeptides (which formed the single antigen binding site) targeted CD19 (Binding Protein 3=CD19×(CD28×CD3)). Binding Protein 5 was constructed such that the first pair of polypeptides (which formed two antigen binding sites) targeted CD28 and CD3, and the second pair of polypeptides (which formed the single antigen binding site) targeted CD38 (Binding Protein 5=CD38×(CD28×CD3)). Binding Protein 6 was constructed such that the first pair of polypeptides (which formed two antigen binding sites) targeted CD28 and CD3, and the second pair of polypeptides (which formed the single antigen binding site) targeted CD38 (Binding Protein 6=CD38×(CD28×CD3)).

### In Vitro Assays Using Trispecific Binding Proteins Comprising Anti-Her2

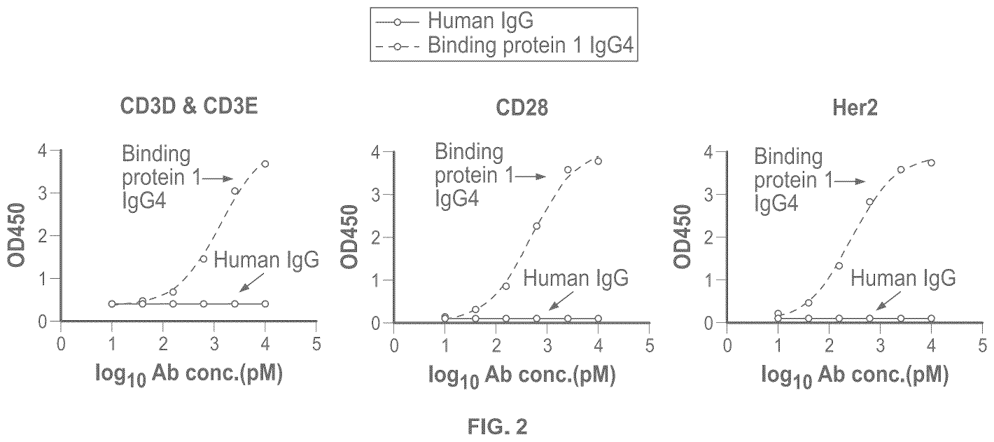

To test the ability of the trispecific binding proteins to target and bind three different human antigens, the specificity of Binding Protein 1 for its targets was first examined by ELISA assay. Binding Protein 1 was capable of binding all three of its target proteins—CD3, CD28, and Her2 (FIG. 2)—indicating that each binding domain in the trispecific format retained its function.

ZR-75-1, AU565 (Her2⁺), ARH-77 (CD19⁺), MOLP-8, RPMI-8226, KMS-12_BM, NCI-H929, MM.1.S, MM.1., R OPM-2, KMS-26, and U266 cells (CD38⁺) were labeled with the membrane dye PKH-26 (Sigma) and used as target cells in a cytotoxicity assay. These labeled cell lines were co-cultured at an E:T ratio of 10:1 with enriched human Pan T cells in the presence of increasing concentrations of a trispecific antibody, bispecific antibody, or control proteins for 24 hours. The extent of cell lysis in the target cells was determined by staining with a live/dead cell marker (Life Technologies) and measuring the number of dead cells in the labeled target cell population by running the samples on a Fortessa flow cytometer (Beckton Dickinson, San Jose, CA) followed by analysis using the Flowjo software (FlowJo v10).

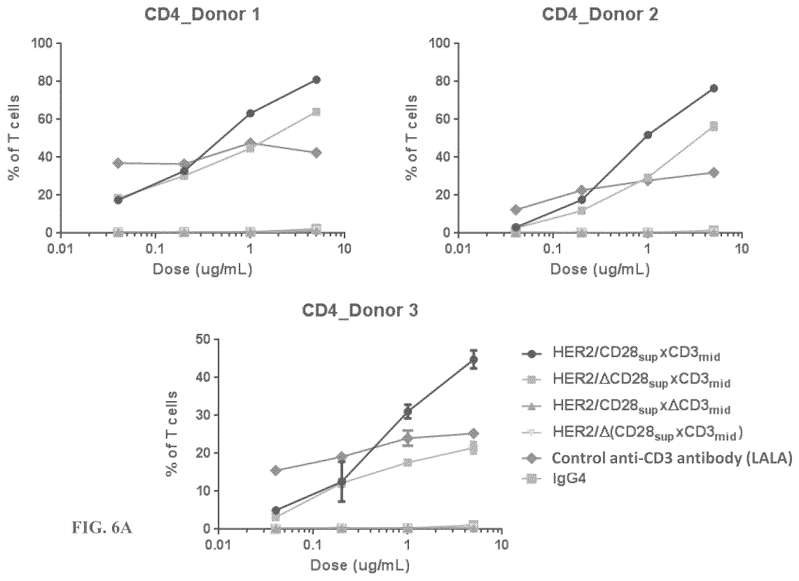

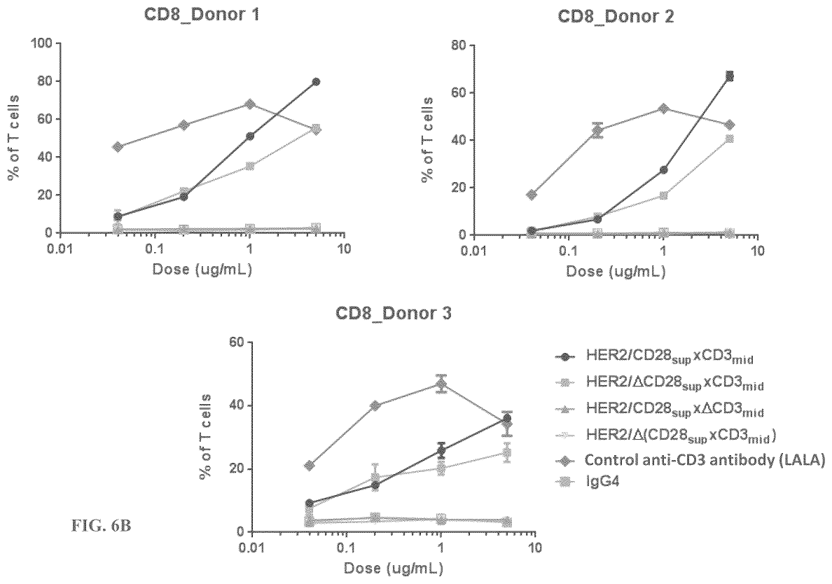

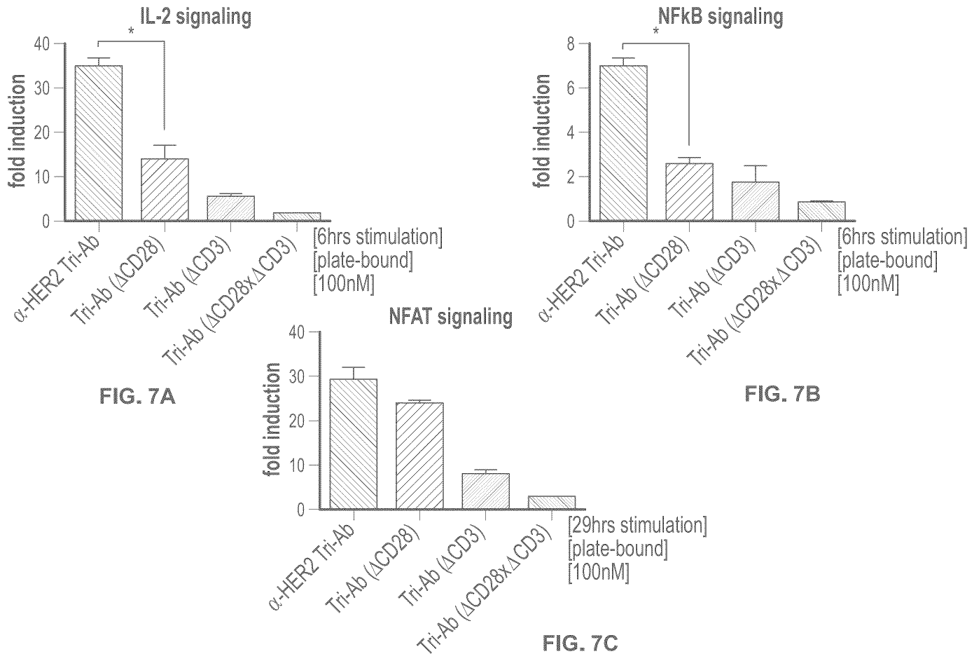

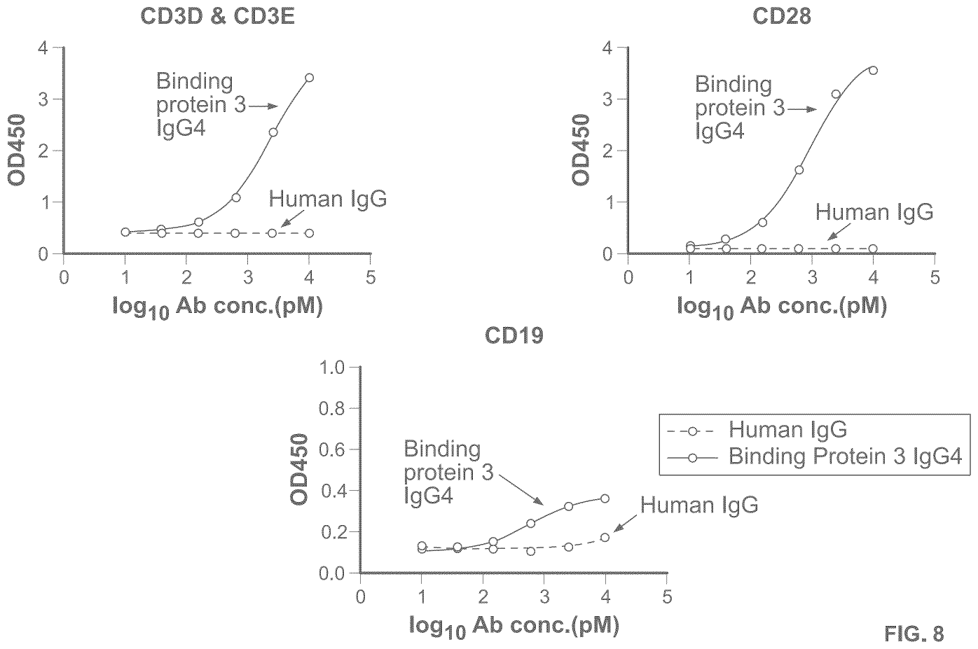

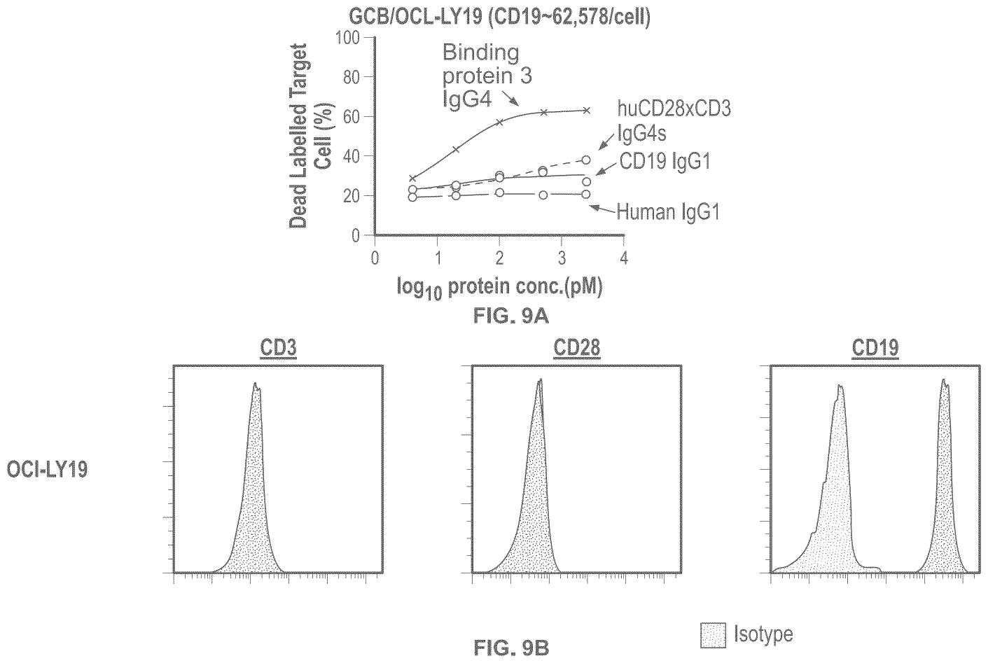

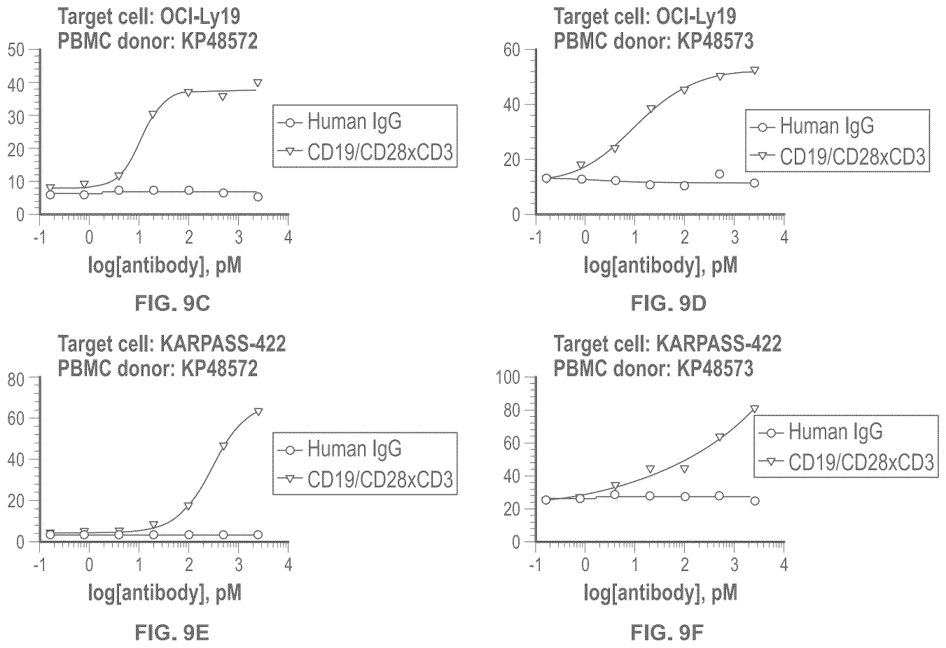

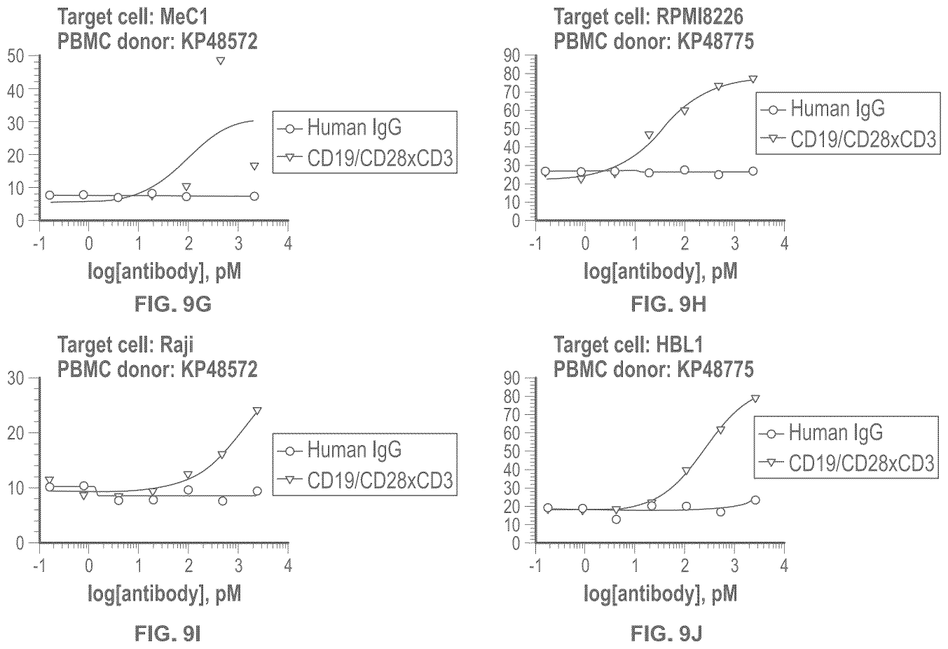

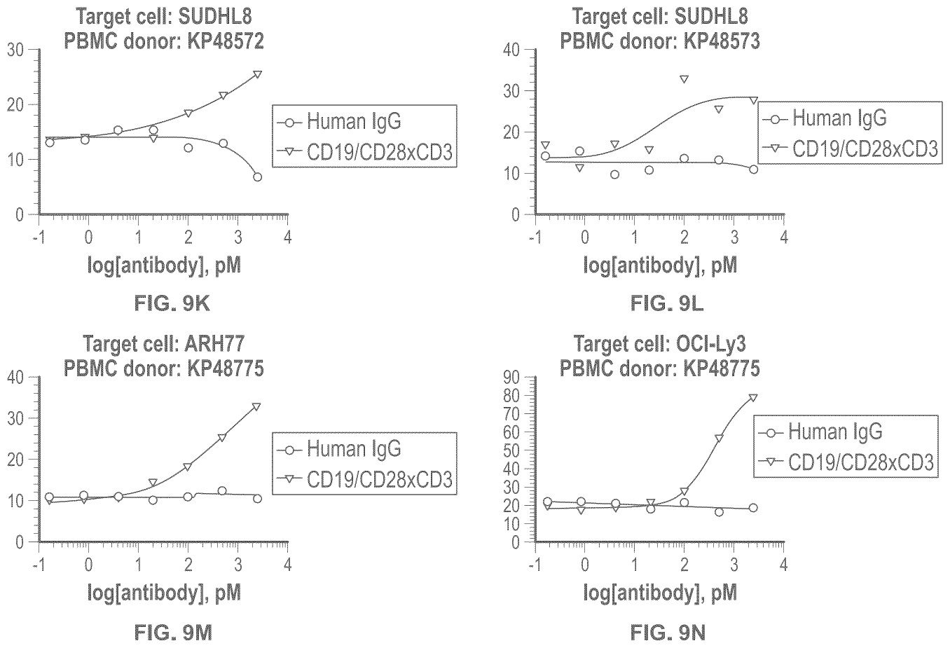

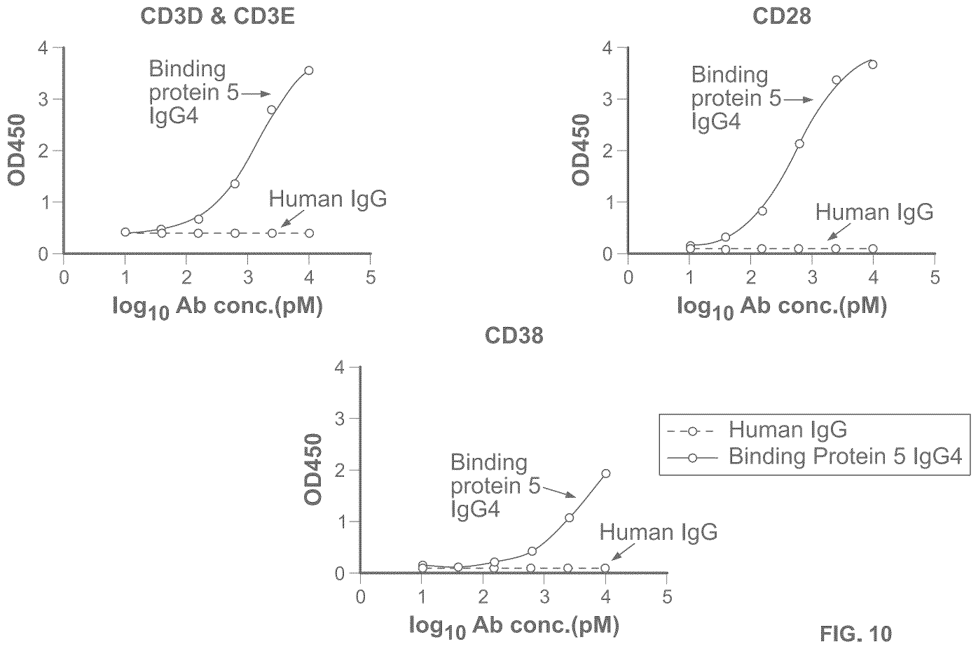

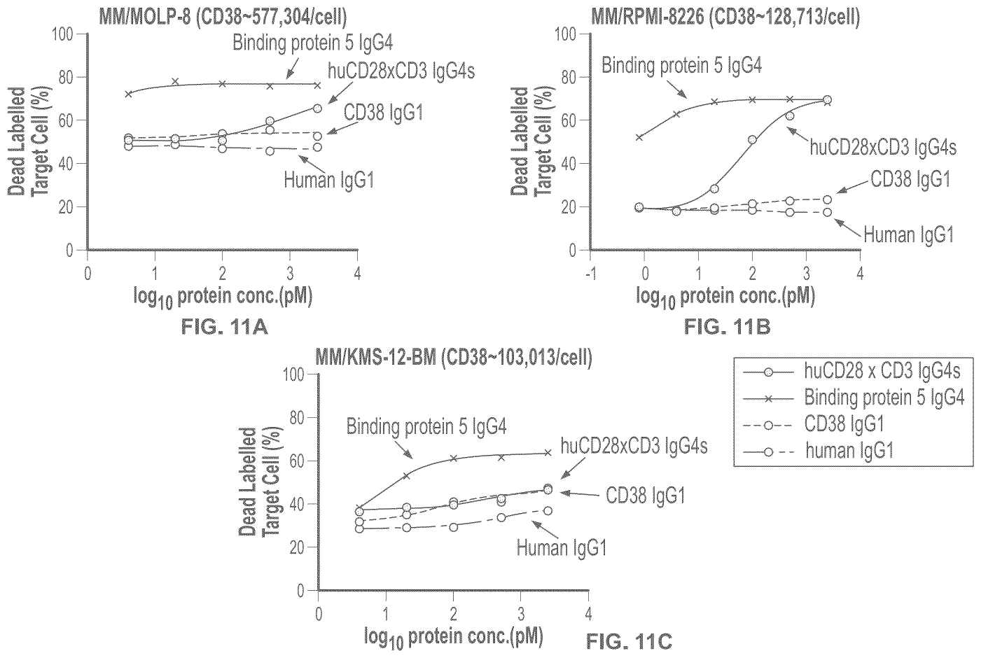

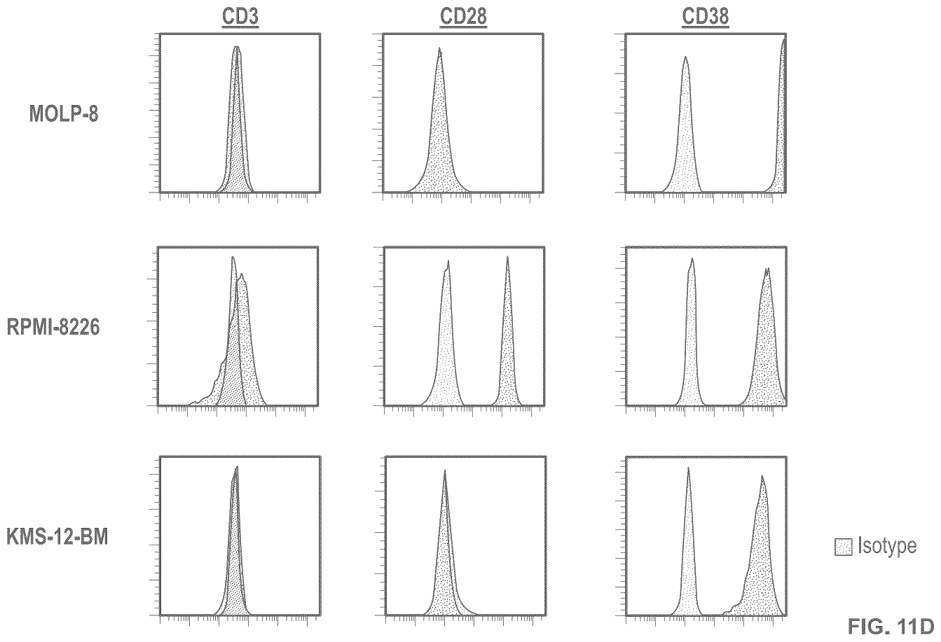

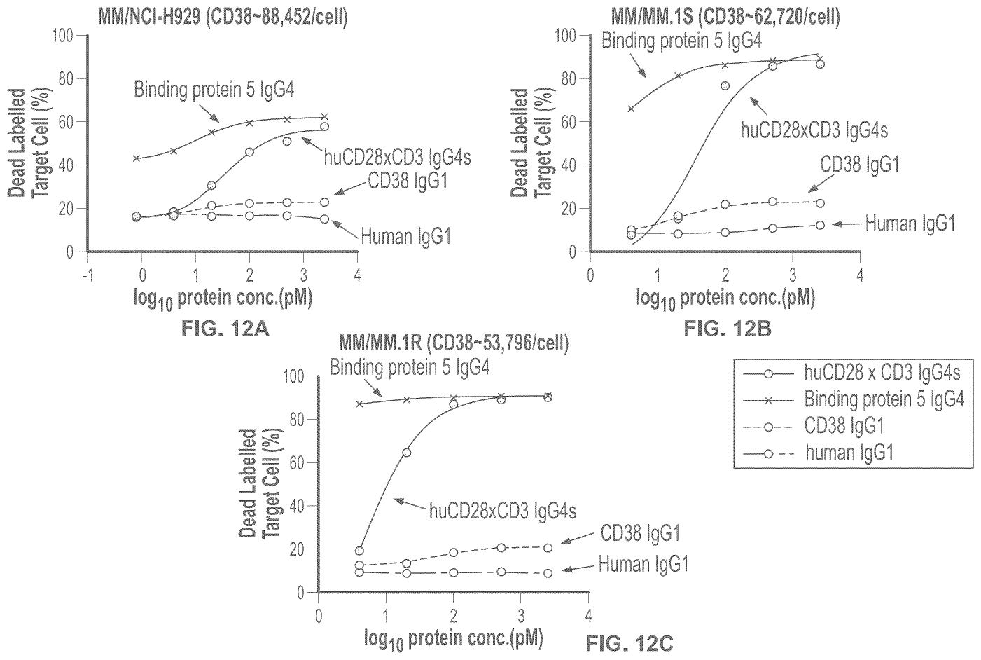

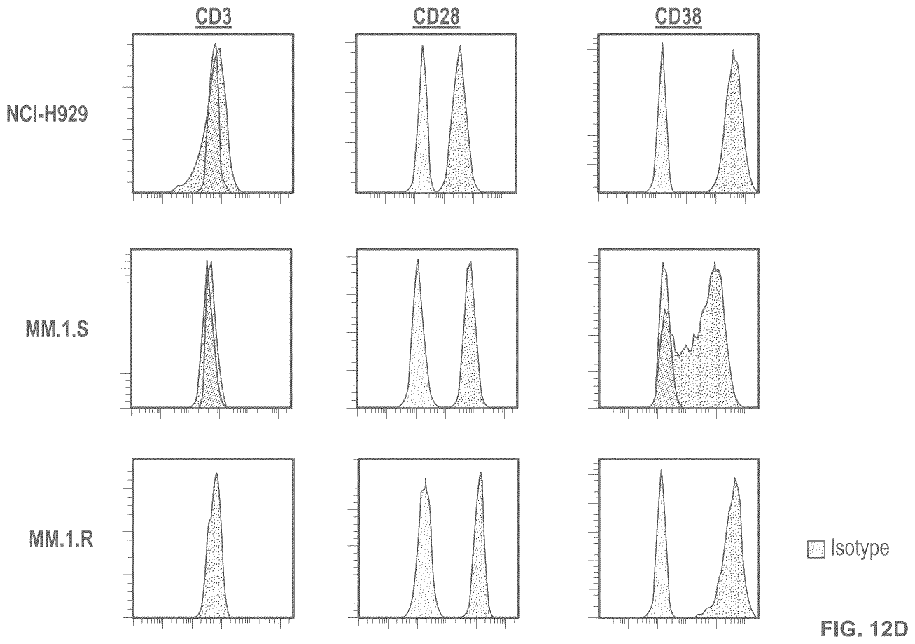

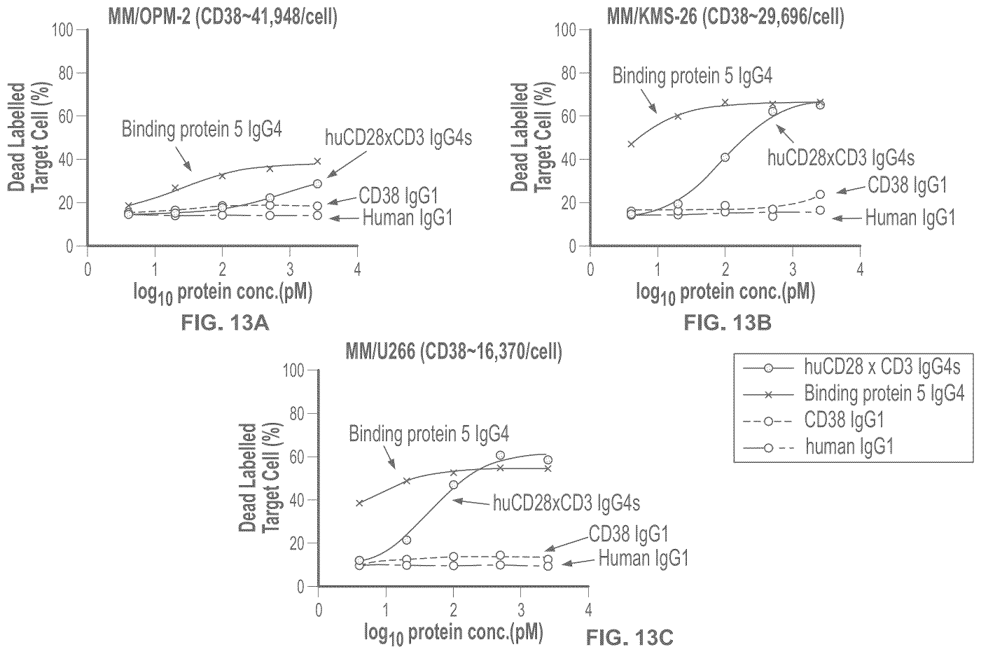

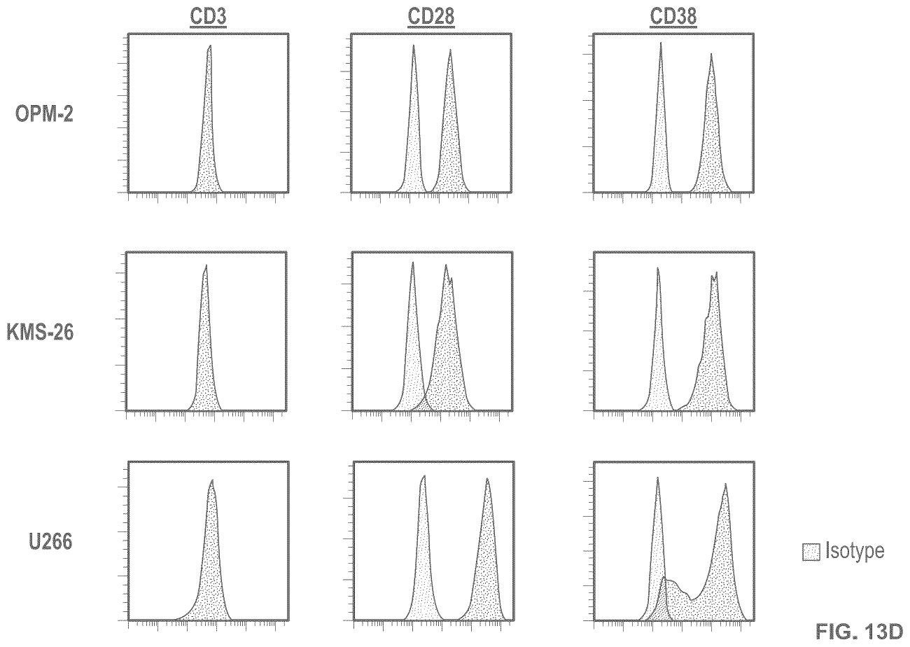

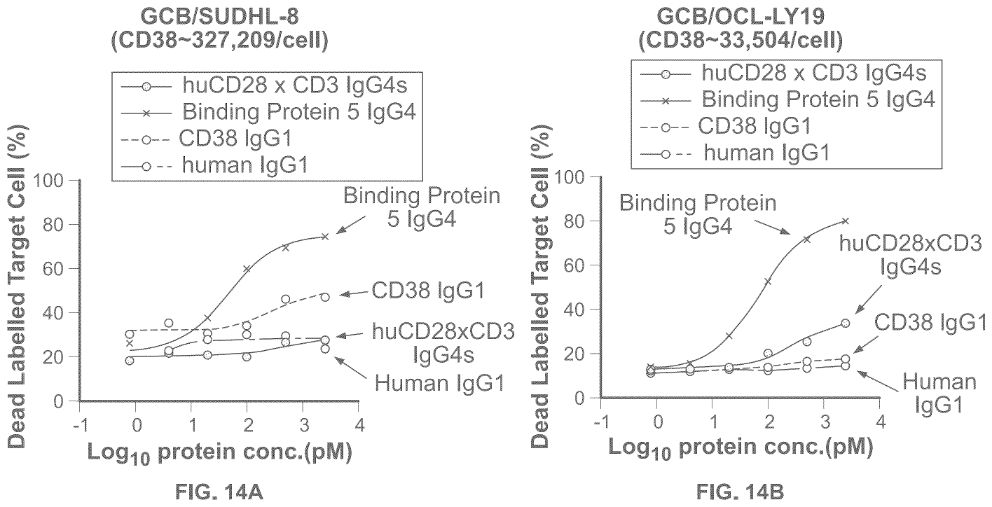

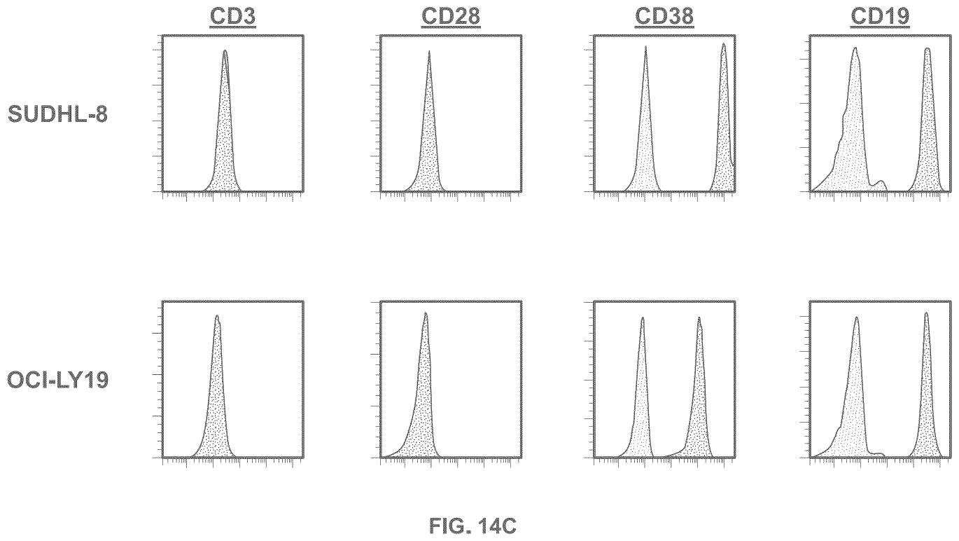

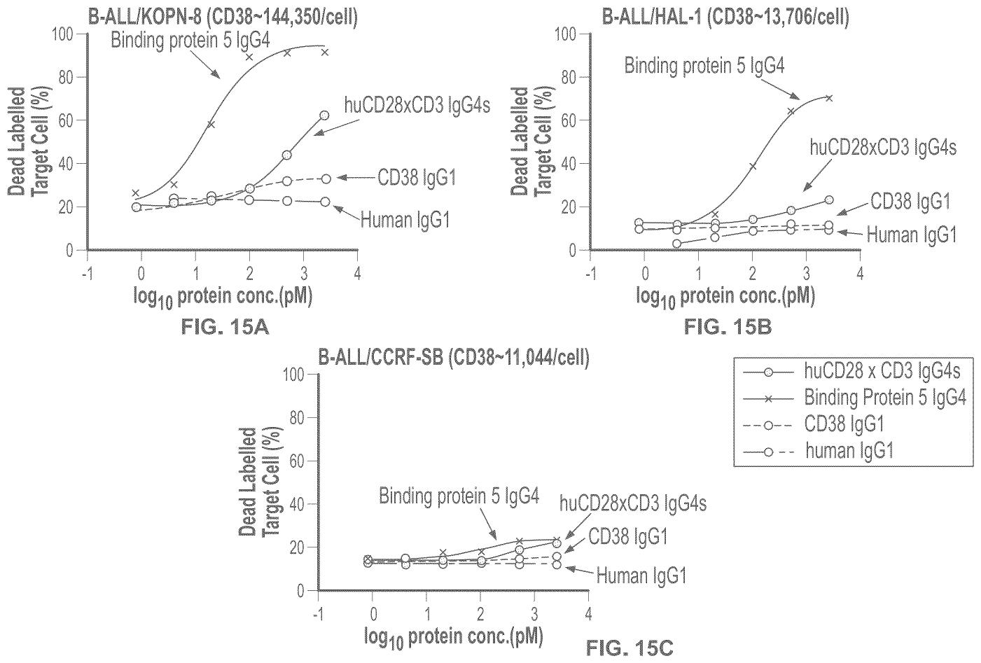

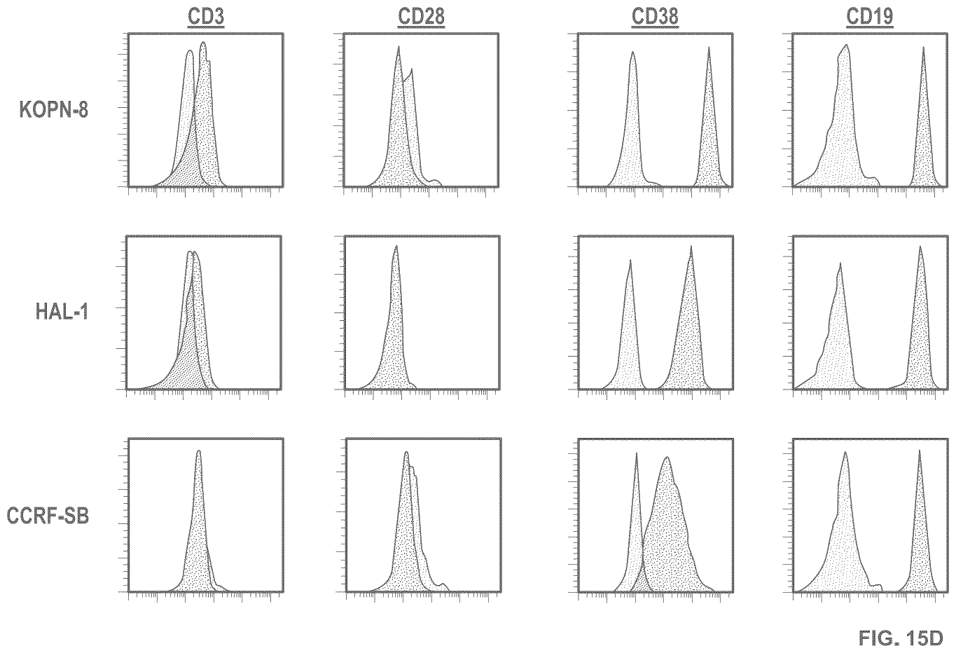

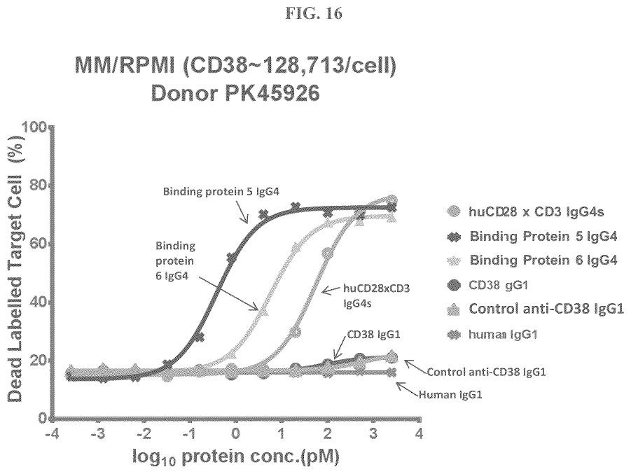

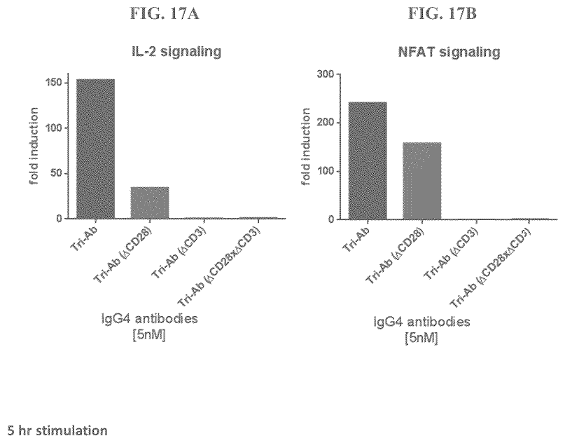

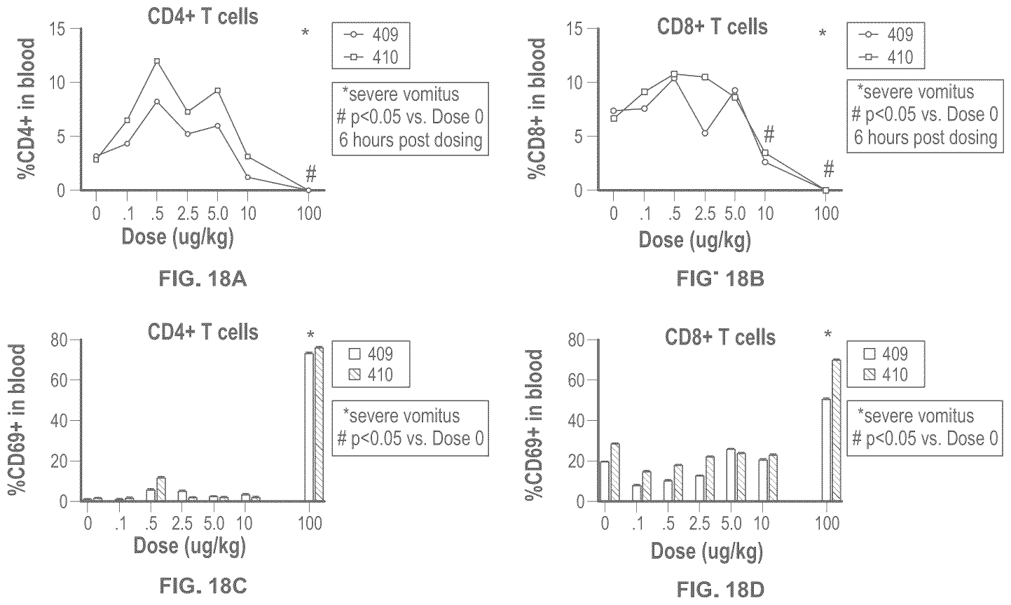

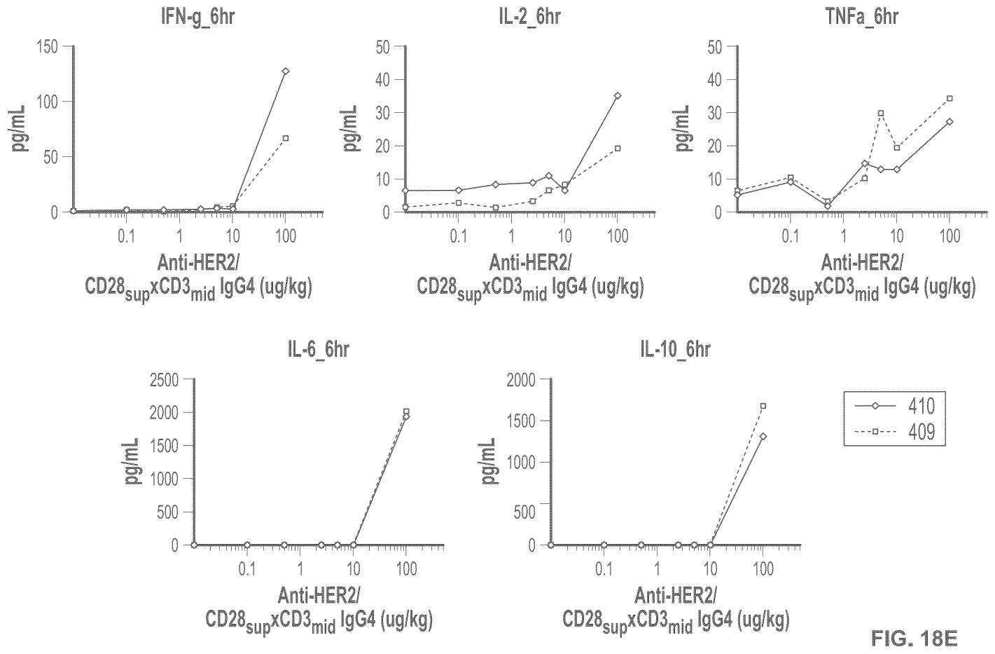

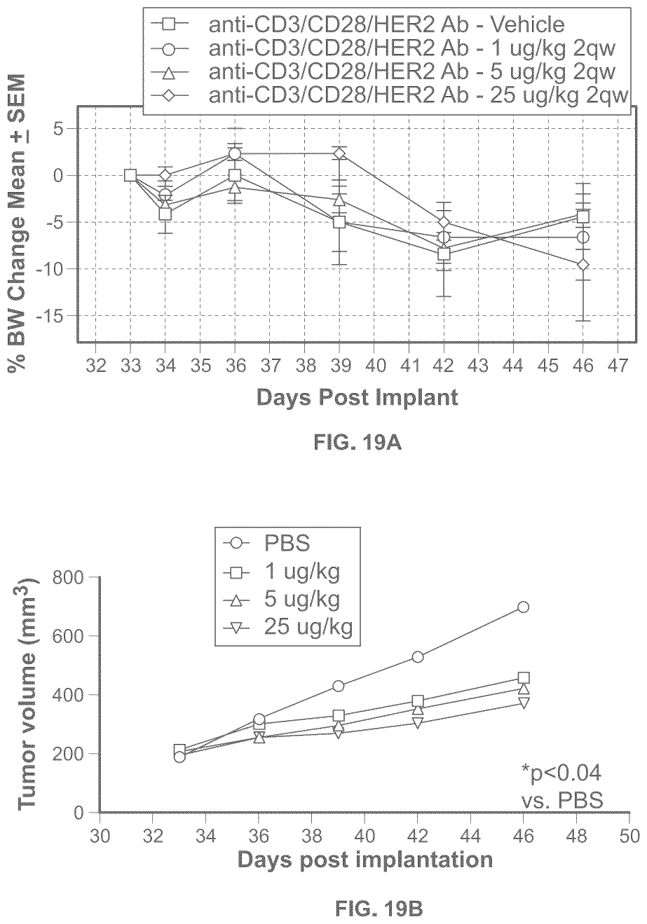

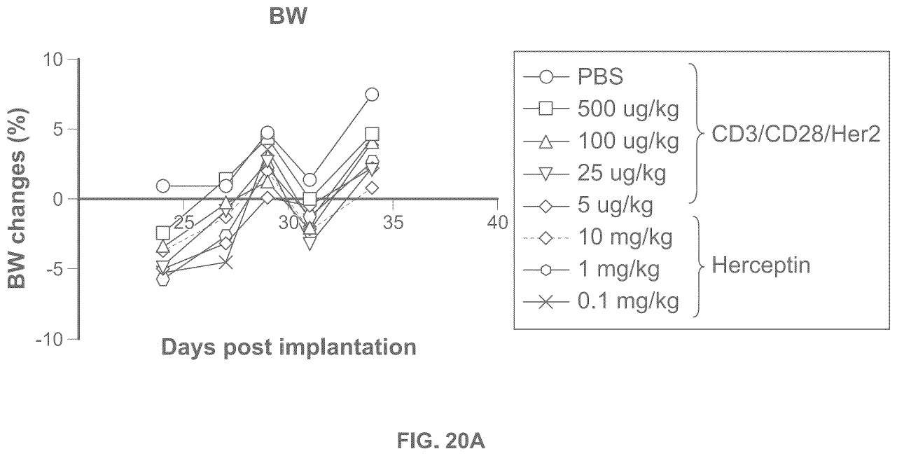

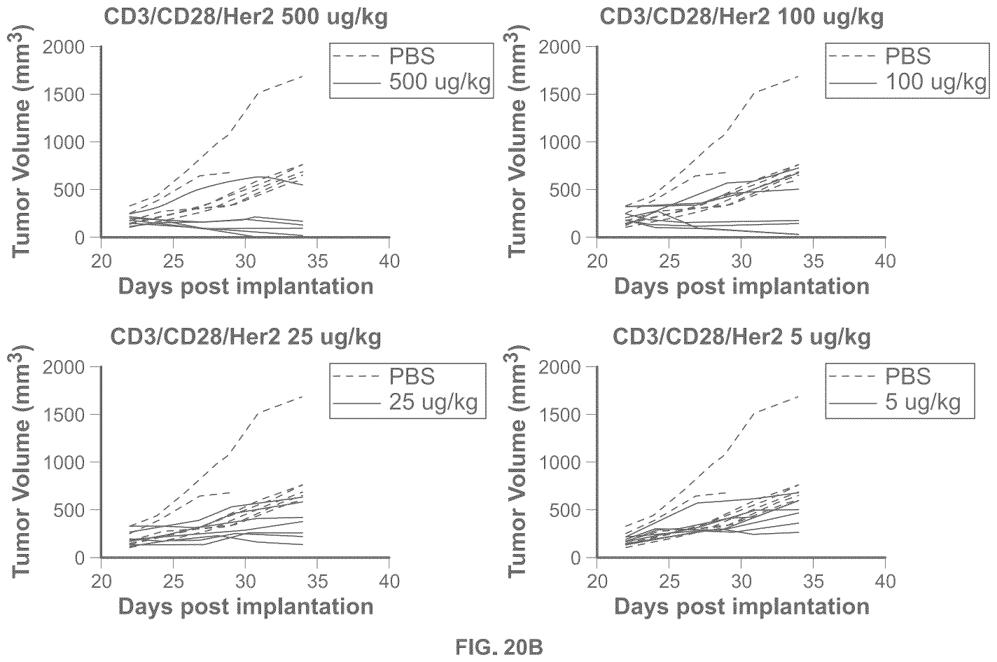

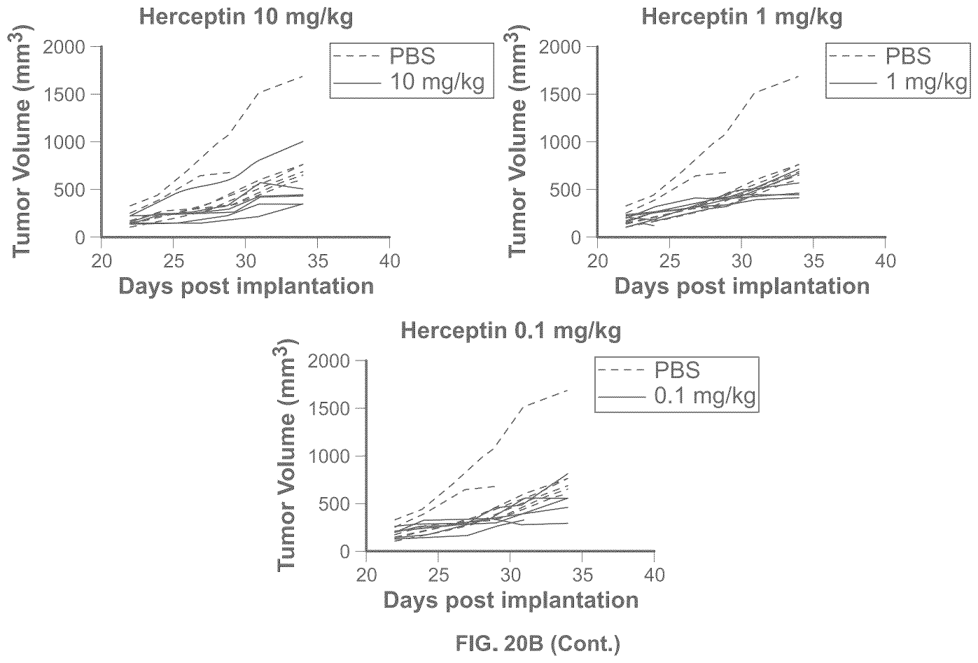

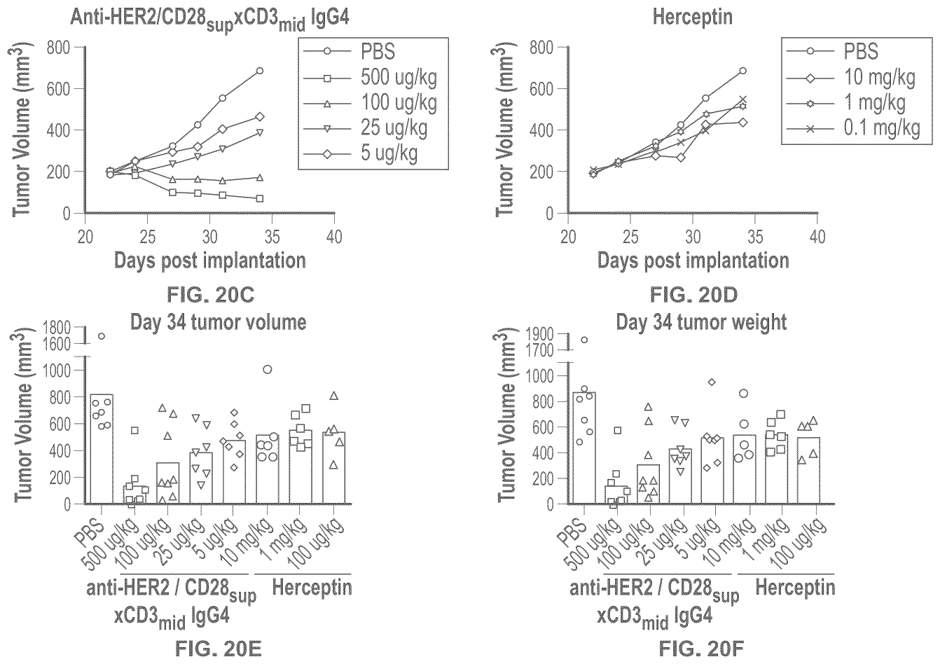

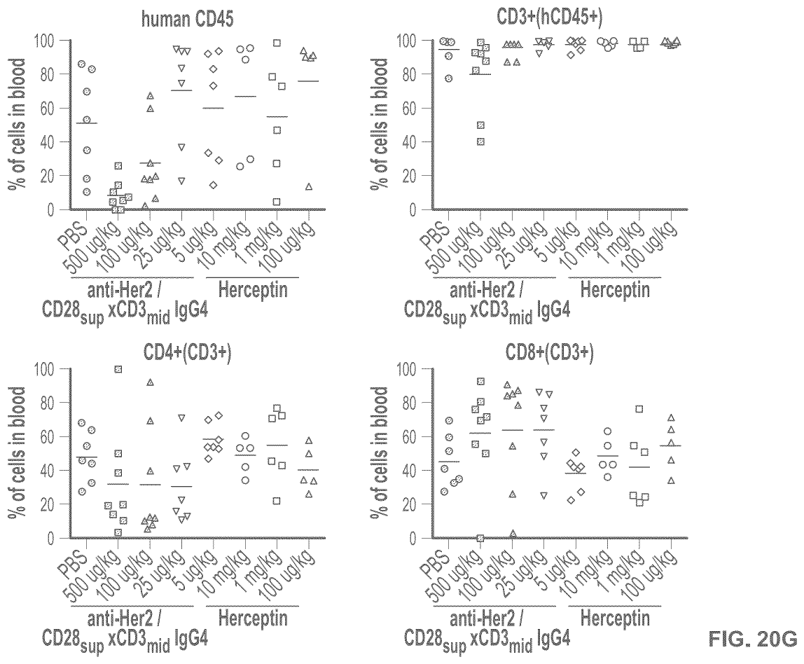

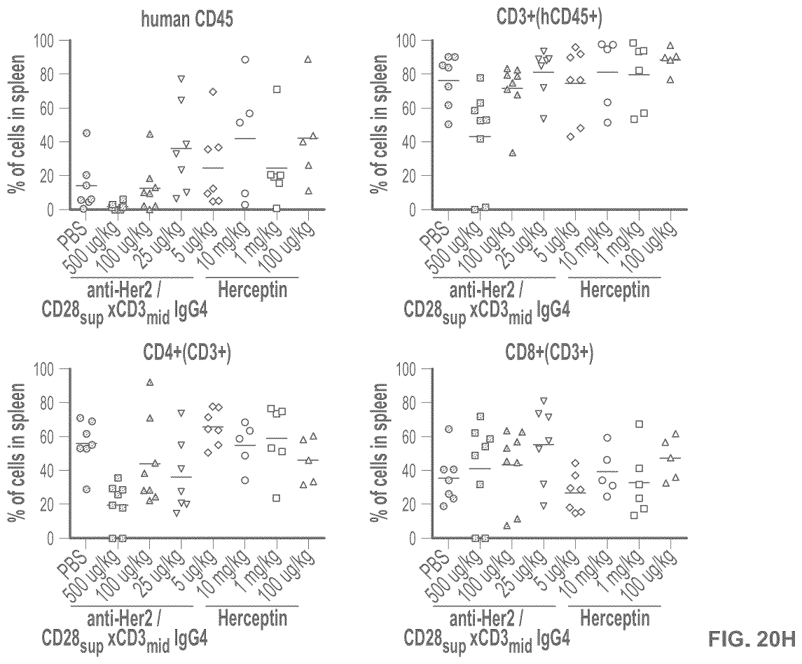

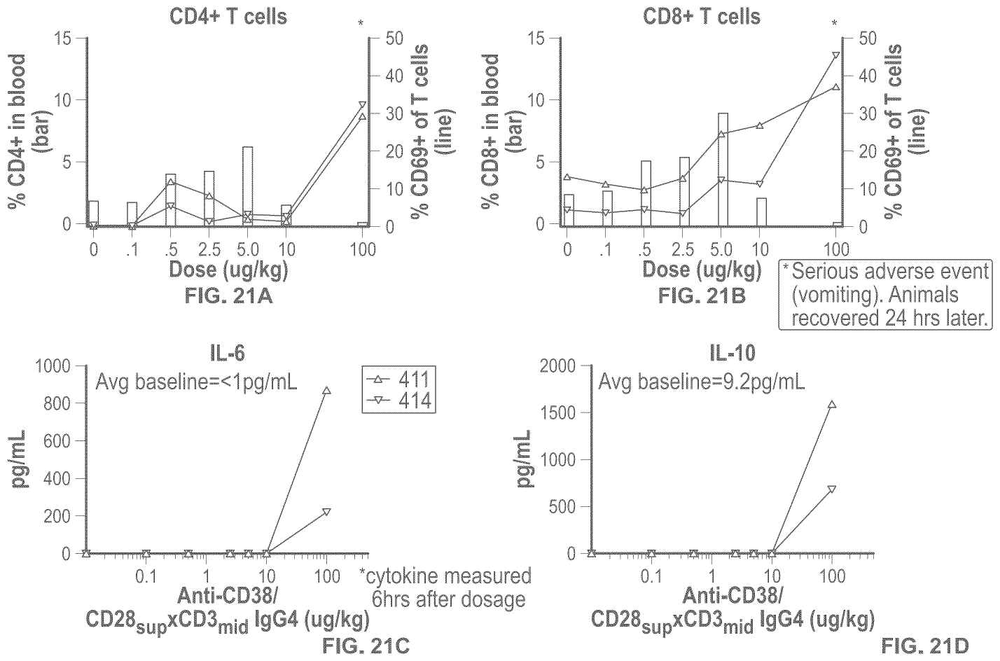

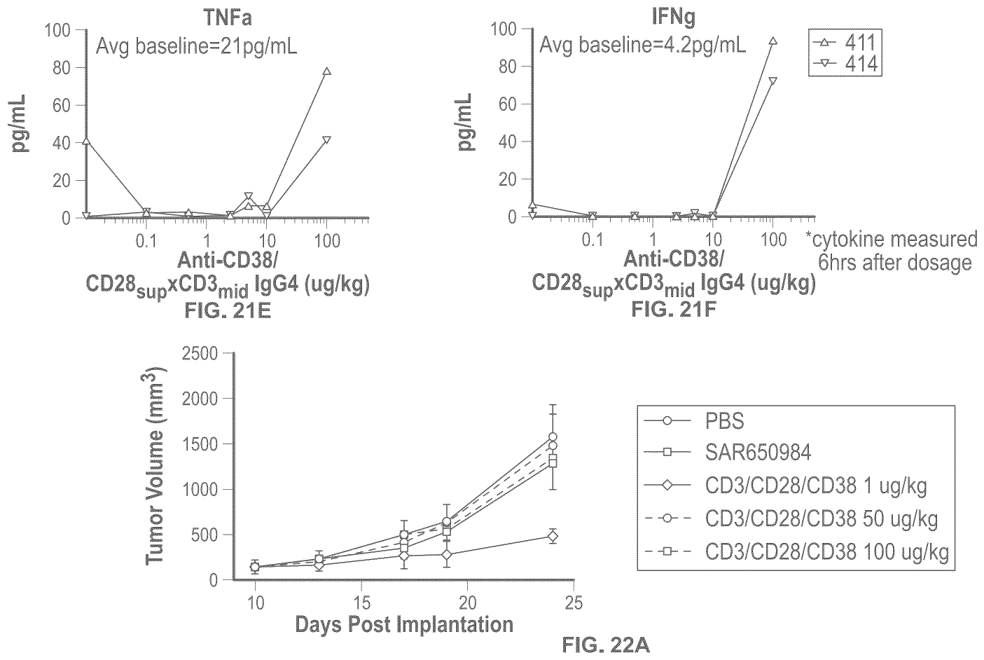

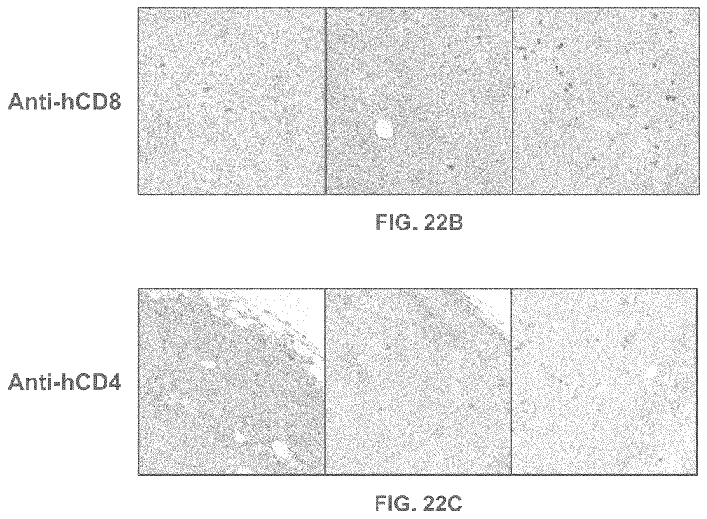

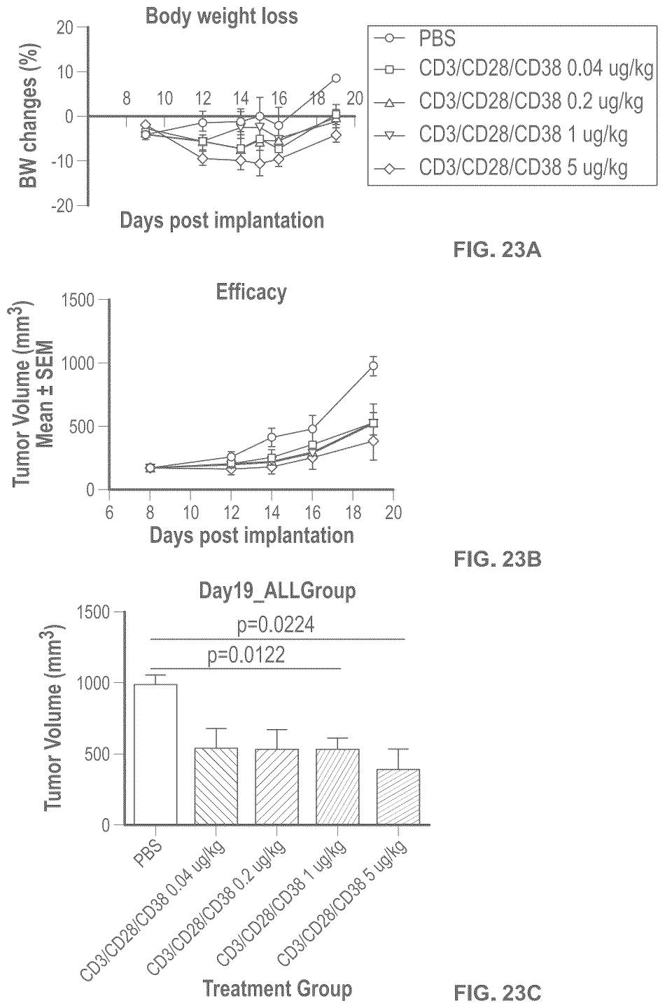

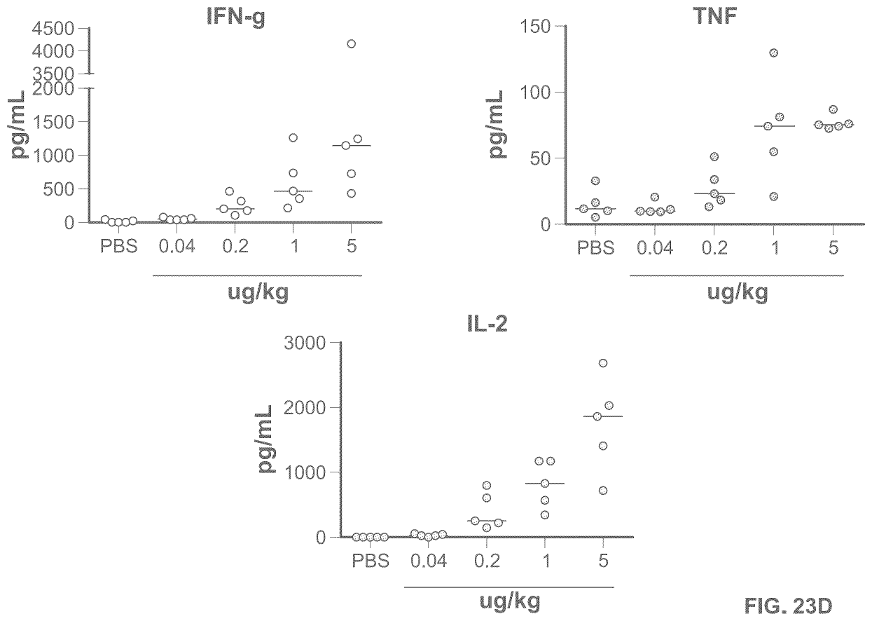

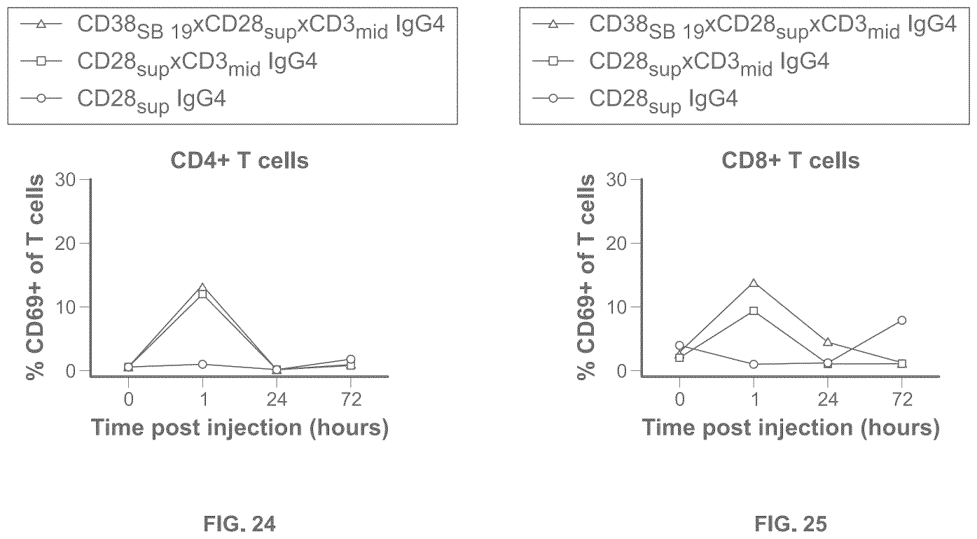

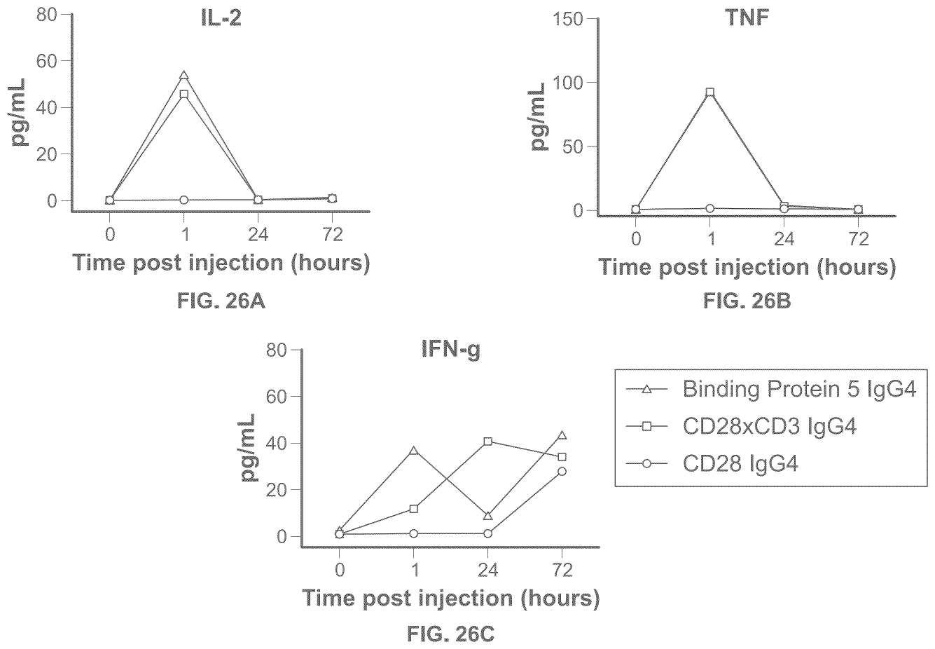

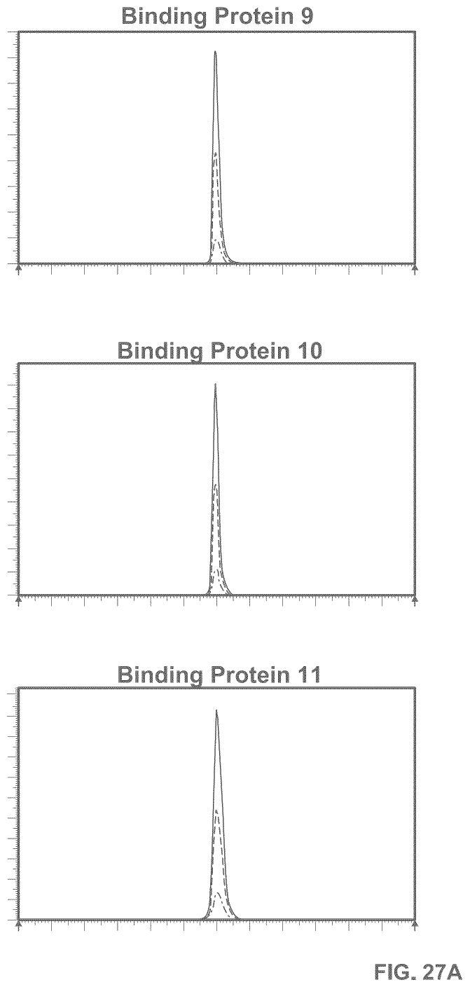

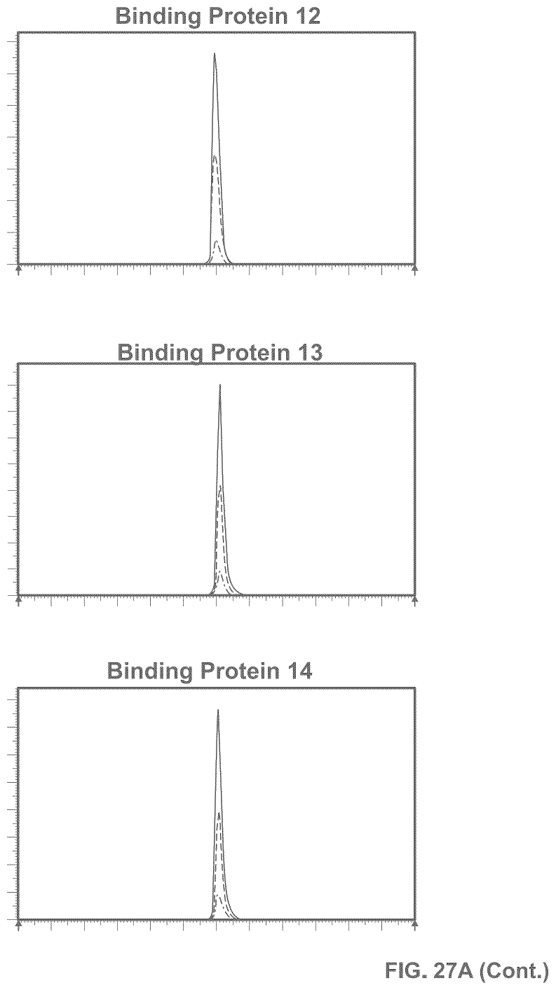

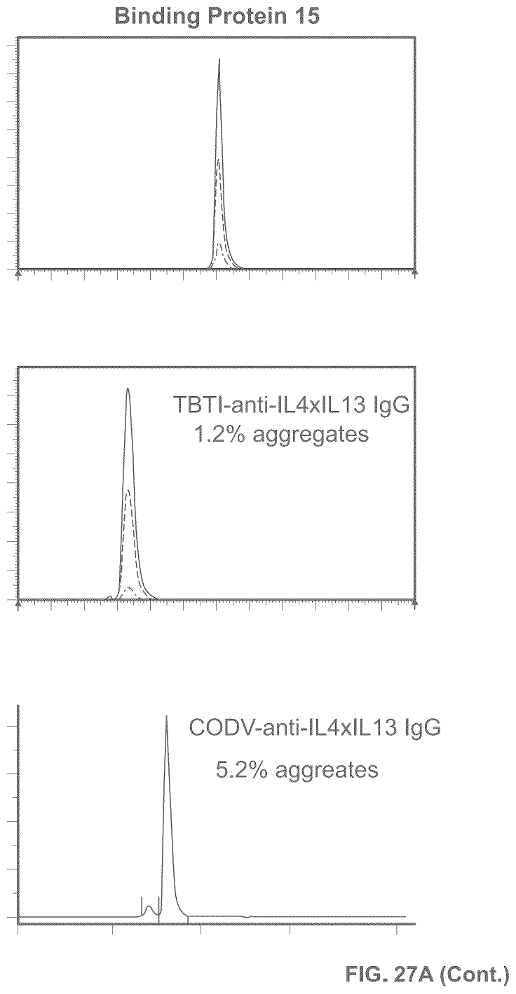

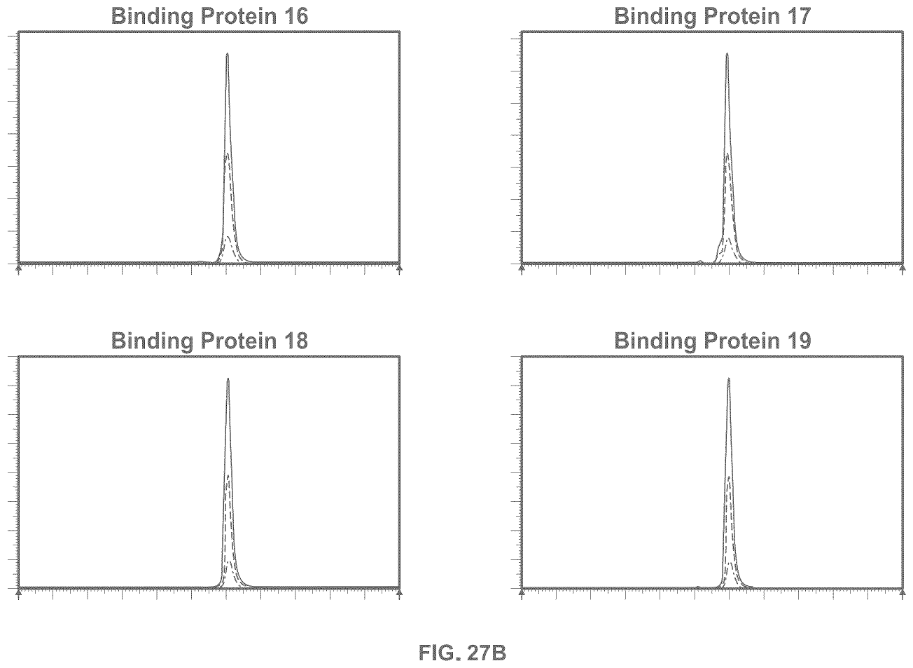

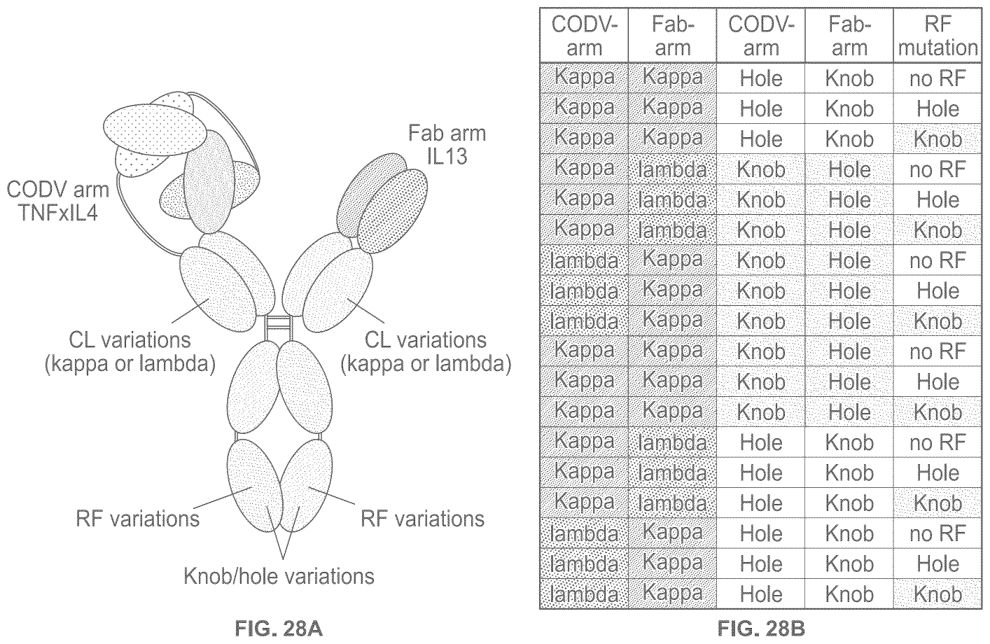

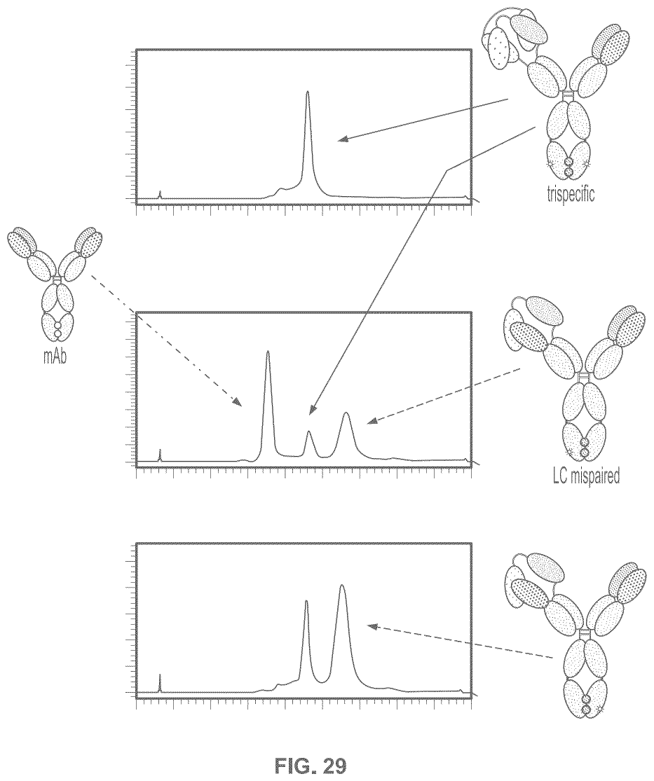

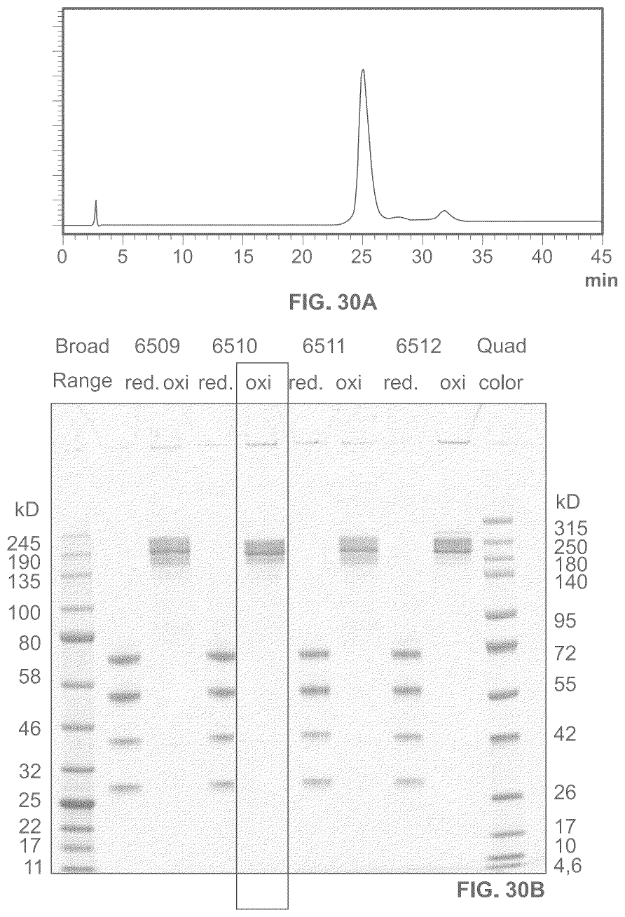

Her2+, CD19+, CD38+ tumor cell lines were stained with fluorescently conjugated antibodies against human CD3, CD28, CD19, CD38, LAMP1, and/or Her2 (Biolegend). Staining with respective isotype-matched control antibodies was also included. The cells were then acquired on the Fortessa (Beckton Dickinson, San Jose, CA) instrument. Flow analysis was performed on FlowJo v10. The mediated killing results of various binding proteins are shown in FIGS. 3A-5, 9A, 9B, & 11A-16.

The ability of Binding Protein 1 to induce antibody-mediated cell killing of tumor cells expressing HER2 proteins on their surface was tested. Not only was Binding Protein 1 capable of binding to all three of its target proteins, but it was also able to induce antibody-mediated cell killing of Her2⁺ cell lines (FIGS. 3A-4). Binding Protein 1 exhibited potent antibody-mediated cell killing activities, while anti-CD3/CD28 bispecific Ab and anti-Her2 antibodies showed minimal killing activities. (FIGS. 3A, 3B, 4, & 5), demonstrating the effectiveness of using the trispecific Ab to engage tumor cells with T cells through a tumor antigen (HER2) and T cell markers (CD3 and CD28). Anti-CD3/CD28 is not only important for T cell recruitment, but it also provides more effective T cell activation and survival signaling, potentially improving the efficacy.

Additionally, studies were carried out on in vitro T cell activation and proliferation, as well as cytokine production, using the anti-Her2×CD28×CD3 trispecific antibody (Binding Protein 1). Binding protein 1 and control variants having one or two binding domains inactivated by site-directed mutagenesis (ΔCD28: anti-CD28 inactivated; ΔCD3: anti-CD3 inactivated; Δ(CD3×CD28): both anti-CD3 and anti-CD28 inactivated) were used in human PBMC in vitro activation assay as described in Example 1. The results showed that Binding protein 1 activated both human primary CD4 T cells and CD8 T cells effectively in vitro. Inactivation of anti-CD28 reduced the activation potency, indicating the importance of anti-CD28 co-signaling pathway. Inactivation of anti-CD3 binding site rendered Binding protein 1 to minimal activity, suggesting that the anti-CD3 provided the primary T cell activation signaling (FIGS. 6A & 6B). Similar results were obtained using IL2 and NFAT reporter human T cell lines (Jurkat-IL2 and Jurkat-NFAT) (FIGS. 7A-7C).

### In Vitro Assays Using Trispecific Binding Proteins Comprising Anti-CD19

The anti-CD19×CD28×CD3 trispecific binding protein was capable of binding its target antigens (FIG. 8), indicating that each binding domain in the trispecific format retained its function.

The anti-CD19×CD28×CD3 trispecific binding protein was also capable of inducing antibody-mediated cell killing of CD19+ cells (FIGS. 9A-9N). Similarly, anti-CD19×CD28×CD3 trispecific binding protein exhibited potent killing activity against human lymphoma cells, while both the anti-CD3/CD28, anti-CD19, and isotype control antibodies showed minimal killing activities, demonstrating the effectiveness of using the trispecific Ab to engage tumor cells with T cells through a tumor antigen (CD19) and T cell markers (CD3 and CD28).

### In Vitro Assays Using Trispecific Binding Proteins Comprising Anti-CD38

As observed with Binding Proteins 1 and 3, Binding Protein 5 was able to bind all three of its target proteins (CD3, CD28, and CD38), as assessed by ELISA assay (FIG. 10), indicating that each binding domain in the trispecific format retained its function.

Binding Protein 5 was also found to induce antibody-mediated cell killing of cells (FIGS. 11A-15D) against 9 human multiple myeloma cells with various levels of CD38 and CD28 expression (see FIGS. 11D, 12D, & 13D). Similarly, trispecific Binding protein 5 exhibited potent killing activity against human multiple myeloma cells, while both the anti-CD38 and isotype control antibodies showed minimal killing activities, demonstrating the effectiveness of using the trispecific Ab to engage tumor cells with T cells through tumor antigens (CD38 and CD28) and T cell markers (CD3 and CD28). Bispecific anti-CD3/CD28 control antibody also showed marginal killing activity against CD28+MM cells see FIGS. 11B, 12A-C, & 13A-C).

These results demonstrate that the trispecific antibody platform described herein provides the possibility of integrating binding sites for two tumor markers, or two binding sites for T cell markers, allowing flexibility for scientific designs and various applications. Binding Protein 5 was also effective against 5 CD38+ human lymphoma cell lines (FIGS. 14C & 15D), showing potent killing activities (FIGS. 14A-B & 15A-C).

The antibody-mediated cell killing against multiple myeloma cell line RPMI8226 using Binding Proteins 5 and 6 were tested, and their EC₅₀s were calculated and compared to that of a CODV format bispecific antibody targeting CD28 and CD3 (FIG. 16 and Table B). Binding proteins 5 and 6 differ only in anti-CD28 binding domain; Binding protein 5 contains an anti-CD28 superagonist, while Binding protein 6 contains a conventional anti-CD28. Binding protein 5 showed more potent killing activity.

TABLE B
EC₅₀ values calculated for bispecific
and trispecific binding proteins

| | EC₅₀ (pM) |
|---|---|
| huCD28 × CD3 IgG4 | 56.16 |
| Binding Protein 5 IgG4 | 0.3787 |
| Binding Protein 6 IgG4 | 5.709 |

The activity of the anti-CD38×CD28×CD3 trispecific binding protein 5 and control variants having one or two binding domains inactivated by site-directed mutagenesis (ΔCD28: anti-CD28 inactivated; ΔCD3: anti-CD3 inactivated; Δ(CD3×CD28): both anti-CD3 and anti-CD28 inactivated) were tested using IL2 and NFAT reporter human T cell lines (Jurkat-IL2 and Jurkat-NFAT) in the in vitro activation assay as described in Example 1. The results showed that Binding protein 5 activated both human IL2 and NFAT promoters effectively in vitro (FIGS. 17A & 17B). Inactivation of anti-CD28 reduced the activation potency, which was more prominent for IL2 reporter, indicating the importance of anti-CD28 co-signaling pathway. Inactivation of anti-CD3 binding site rendered Binding protein 5 to minimal activity, suggesting that the anti-CD3 provided the primary T cell activation signaling.

### Example 4: In Vivo Activity of the T Cell Engagers

This example describes experiments characterizing the properties and activities of the anti-Her2 or anti-CD38 containing T cell engagers in vivo.

### In Vivo Assays Using Trispecific Binding Proteins Comprising Anti-Her2

A dose escalation study using the Her2×CD28×CD3 trispecific antibody was carried out in non-human primates (FIGS. 18A-18E) as described in Example 1. All three binding domains in Binding protein 1 are cross-reactive with monkey CD3/CD28/HER2. A dose escalation toxicity study was devised to assess the potential toxicity profile of the molecular. Blood samples were collected for serum and PBMC isolations. Circulating T cell populations were investigated after each dosing (FIGS. 18A & 18B), along with T cell subpopulation activation (CD69+) (FIGS. 18C & 18D). Percentage of CD4 and CD8 T-cells in circulation were increased at low dose escalation, but eventually decreased at high dose escalation. Significant CD4 and CD8 T cell activation were only prominent at 100 μg/kg dose, suggesting rather a relative high tolerable dose. Serum level of several cytokines were also measured. Significant cytokine release was only observed at the highest dose (100 μg/kg; FIG. 18E).

Next, the effect of the trispecific anti-Her2×CD28×CD3 Binding protein 1 antibody on tumor growth in humanized mouse models was examined as described in Example 1 (FIGS. 19A-20H). FIGS. 19A & 19B summarize the results obtained using the human CD34+ hematopoietic stem cell-engrafted NSG mice (hu-CD34) model inoculated with human HER2+ breast cancer line BT474. Significant anti-tumor activities were evident within all dose groups. The anti-tumor activity was dose dependent, which is statistically different compared to the control group at 25 μg/kg. No significant body weight loss in any treated groups observed.

A 2^{nd} in vivo study using human PBMC reconstituted NSG mice model inoculated with human HER2+ breast cancer line BT474 was also done (FIGS. 20A-20H). Significant anti-tumor activities were observed within high dose groups (100 and 500 μg/kg). Tumor shrinkage was seen in 40% of the mice in 500 μg/kg group. The anti-tumor activity was dose dependent. The anti-tumor activity in groups treated with 100 and 500 μg/kg doses were significantly better than anti-HER2-treated groups (0.1 to 10 mg/kg), indicating superior anti-tumor activity from Binding protein 1. No significant body weight loss in any treated groups observed.

### In Vivo Assays Using Trispecific Binding Proteins Comprising Anti-CD38

A dose escalation study was conducted in non-human primates using the trispecific anti-CD38×CD28×CD3 antibody (Binding protein 5) as described in Example 1 (FIGS. 21A-21F). Two of the three binding domains in Binding protein 5 are cross-reactive with monkey CD3 and CD28. A dose escalation toxicity study was devised to assess the potential toxicity profile of the molecule. Blood samples were collected for serum and PBMC isolations. Circulating T cell populations were investigated after each dosing (FIGS. 21A & 21B, bar graphs), along with T cell subpopulation activation (CD69+) (FIGS. 21A & 21B, line graphs). Percentage of CD4 and CD8 T-cells in circulation increased at low dose escalation, but eventually decreased at high dose escalation. Significant CD4 and CD8 T cell activation were only prominent at 100 μg/kg dose, suggesting rather a relative high tolerable dose. Serum level of several cytokines was also measured. Significant cytokine release was only observed at the highest dose (100 μg/kg; FIGS. 21C-21F).

The in vivo activity of the anti-CD38×CD28×CD3 trispecific antibody was next tested in humanized mice (FIGS. 22A-23D) as described in Example 1. FIGS. 22A-22C summarized the result from a dose determining pilot study using the human CD34+ hematopoietic stem cell-engrafted NSG mice (hu-CD34) model implanted with human MM cell line RPMI-8226 transduced with CD38 and PD-L1, treated with Binding protein 5 at doses 5, 50 and 100 μg/kg. Significant anti-tumor activity was only evident in group treated with 5 μg/kg (FIG. 22A). CD8 T cell infiltration was observed in Binding protein 5 treated mice (5 μg/kg) (FIGS. 22B & 22C).

A follow up study in the same model was performed using Binding protein 5 at dosing from 0.04-5 μg/kg (FIGS. 23A-23D). Significant anti-tumor activity was shown in all group treated with Binding protein 5 (FIG. 23B), which were statistically different from the control at the end of study (FIG. 23C). No significant body weight loss was observed in any treated groups (FIG. 23A). Dose dependent induction of serum inflammatory cytokines IFN-7, TNF-α and IL-2 four hours after the first dose was observed in mice treated with indicated concentrations of the Binding protein 5 or PBS control (FIG. 23D), indicating effective T cell activation by trispecific Binding protein 5 in vivo.

Humanized CD34+ NSG mice (n=3) were injected i.v. with 100 mcg/kg of Trispecific Ab (triangel), Bi-specific Ab (square), or single-specific Ab (circle). Activation of CD4+ or CD8+ T cells was measured at 0 (pre-injection), 1, 24, and 72 hours after Ab injection by determining mean increase in % of CD69, decrease % of CD62L and/or concentration of inflammatory cytokines in plasma at each time points by Luminex's xMAP multiplexing technology. The T cell activation results of various trispecific antibodies are shown in FIGS. 24-26C.

Systemic in vivo T cell activation was studied in human CD34+ hematopoietic stem cell-engrafted NSG mice (hu-CD34) model after administration of Binding protein 5, anti-CD3/CD28_IgG4 bispecific antibody and anti-CD28 IgG4 antibody controls (FIG. 24). 100 μg/kg of the Binding protein 5 and control antibodies were administered into 3 mice/group. Blood samples were collected at pre, 1 hour, 24 hours and 72 hours post administration. Mouse sera and human T cells were isolated from blood, and preserved for T cell activation analysis and for measurement of serum cytokine level. FIGS. 24 & 25 show that both human CD4 and CD8 T cell were activated 1 hour post antibody infusion, which returned to baseline at 72 hours. FIGS. 26A-26C shows the elevation of serum IFN-7, TNF-α and IL-2 release in the same mice, which was observed 1 hour post infusion, and returned to baseline 24 hours later. These results demonstrated both Binding protein 5 and anti-CD3/CD28 IgG4 bispecific antibody are effective in activating T cell in the given animal model, making it suitable for in vivo efficacy study.

### Example 5: Characterization of Cytokine-Directed Trispecific and Bispecific-Trivalent Binding Proteins

The follow example describes experiments characterizing the stability, binding properties, and activities of novel trispecific and bispecific-trivalent binding proteins that target human cytokines.

Trispecific binding proteins (e.g., that bind three different target proteins; Binding Proteins 9-15), as well as bispecific-trivalent binding proteins (e.g., that bind one antigen bivalently on one antigen monovalently; Binding Proteins 16-19), were designed (Table C). With the exception of Binding Protein 11 where a kappa constant domain was used on both the CODV-LC and the Fab-arm-LC, all other Binding Proteins (9-10 and 12-19) were produced with a kappa constant domain on the CODV-LC and a lambda constant domain on the Fab-arm-LC. As Fc-backbone the IgG1 sequence was used. Whereas the CODV-HC harbors the knob-RF mutations (S354C, T366W; H435R and Y436F) the Fab-arm-HC contains the hole mutations (Y349C, T366S, L368A, Y407V).

TABLE C
summary of the trispecific/trivalent binding proteins directed
to anti-IL-4/IL-13/TNFα

| Antibody | Specificity | Construct | Format |
|---|---|---|---|
| Binding | (anti-IL4 × | (CODV-Fab) × | Trispecific |
| Protein 9 | anti-IL13) × anti-TNFα | Fab-IgG1 Fc | |
| Binding | (anti-IL13 × | (CODV-Fab) × | Trispecific |
| Protein 10 | anti-IL4) × anti-TNFα | Fab-IgG1 Fc | |
| Binding | (anti-IL13 × | (CODV-Fab) × | Trispecific |
| Protein 11 | anti-IL4) × anti-TNFα | Fab-IgG1 Fc | |
| Binding | (anti-IL4 × anti- | (CODV-Fab) × | Trispecific |
| Protein 12 | TNFα) × anti-IL13 | Fab-IgG1 Fc | |
| Binding | (anti-TNFα × | (CODV-Fab) × | Trispecific |
| Protein 13 | anti-IL4) × anti-IL13 | Fab-IgG1 Fc | |
| Binding | (anti-IL13 × | (CODV-Fab) × | Trispecific |
| Protein 14 | anti-TNFα) × anti-IL4 | Fab-IgG1 Fc | |
| Binding | (anti-TNFα × | (CODV-Fab) × | Trispecific |
| Protein 15 | anti-IL13) × anti-IL4 | Fab-IgG1 Fc | |
| Binding | (anti-IL13 × | (CODV-Fab) × | Bispecific |
| Protein 16 | anti-IL4) × anti-IL13 | Fab-IgG1 Fc | Trivalent |
| Binding | (anti-IL13 × | (CODV-Fab) × | Bispecific |
| Protein 17 | anti-IL4) × anti-IL4 | Fab-IgG1 Fc | Trivalent |
| Binding | (anti-IL4 × | (CODV-Fab) × | Bispecific |
| Protein 18 | anti-IL13) × anti-IL13 | Fab-IgG1 Fc | Trivalent |
| Binding | (anti-IL4 × | (CODV-Fab) × | Bispecific |
| Protein 19 | anti-IL13) × anti-IL4 | Fab-IgG1 Fc | Trivalent |

The trispecific and bispecific-trivalent binding proteins were produced and purified as described above (FIG. 27).

TABLE D
SEC purification of Binding Proteins 16-19

| | Reten- | Peak | | | MW | MW |
|---|---|---|---|---|---|---|
| | tion | Height | Area | Aggregation | by SEC | Calc. |
| Construct | (mL) | (mAU) | (mAU * mL) | (%) | (kDa) | (kDa) |
| Binding | 3.02 | 64.8 | 8.1 | 1.5 | 211 | 11 |
| Protein 16 | | | | | | |
| Binding | 2.99 | 65.9 | 8.9 | 2.1 | 225 | 172 |
| Protein 17 | | | | | | |
| Binding | 3.01 | 72.9 | 8.8 | 0.0 | 214 | 171 |
| Protein 18 | | | | | | |
| Binding | 2.98 | 73.2 | 8.8 | 0.9 | 228 | 171 |
| Protein 19 | | | | | | |

In order to assess the stability of the trispecific binding proteins, their melting point was assessed by DSF and compared with the thermostability of the parental antibodies (Table E).

TABLE E
summary of the thermostability by DSF and percent monomers
from preparative size exclusion chromatography
for various trispecific binding proteins

| | | | Prep SEC |
|---|---|---|---|
| | Tm | | Monomer |
| Construct | (° C.) | | (%) |
| IL4 | 70 | 81 | 100 |
| IL13 | 67 | 78 | 92.5 |
| TNFα | 70 | — | nd |
| Binding | 63 | — | 92.2 |
| Protein 9 | | | |
| Binding | 62 | — | 85.7 |
| Protein 10 | | | |
| Binding | 63 | 70 | 65.7 |
| Protein 11 | | | |
| Binding | 59 | 70 | 87.3 |
| Protein 12 | | | |
| Binding | 59 | 70 | 94.5 |
| Protein 13 | | | |
| Binding | 56 | 69 | 92.4 |
| Protein 14 | | | |
| Binding | 58 | 66 | 92.2 |
| Protein 15 | | | |
| IL13 × IL4 | 63 | 75 | 88.0 |
| IL4 × IL13 | 64 | — | nd |

nd = not determined

To assess the binding affinity of every single antibody binding domain within the trispecific format, SPR analysis for each single antigen was performed as described previously. The results were benchmarked against the affinities of the parental antibodies (Tables F, G, and H).

TABLE F
summary of surface plasmon resonance results for IL-4 for
various trispecific binding proteins

| | Kₐ | K_{d} | K_{D} | | |
|---|---|---|---|---|---|
| Construct | [1/M * s] | [1/s] | [M] | Rmax | Chi^{∧}2 |
| IL4 | 8.70E+07 | 1.57E−04 | 1.81E−12 | 24 | 0.24 |
| IL13 | | | | | |
| TNFα | | | | | |
| Binding | 7.86E+07 | 3.80E−04 | 4.83E−12 | 26 | 0.309 |
| Protein 9 | | | | | |
| Binding | 1.88E+07 | 8.41E−05 | 4.47E−12 | 23 | 0.763 |
| Protein 10 | | | | | |
| Binding | 5.92E+07 | 2.39E−04 | 4.04E−12 | 20 | 0.198 |
| Protein 11 | | | | | |
| Binding | 6.02E+07 | 2.39E−04 | 3.97E−12 | 35 | 0.406 |
| Protein 12 | | | | | |
| Binding | 3.57E+07 | 1.81E−04 | 5.07E−12 | 30 | 0.257 |
| Protein 13 | | | | | |
| Binding | 8.96E+07 | 1.52E−04 | 1.69E−12 | 33 | 0.254 |
| Protein 14 | | | | | |
| Binding | 7.35E+07 | 1.23E−04 | 1.67E−12 | 31 | 0.547 |
| Protein 15 | | | | | |

TABLE G
summary of surface plasmon resonance results for IL-13
for various trispecific binding proteins

| | Kₐ | K_{d} | K_{D} | | |
|---|---|---|---|---|---|
| Construct | [1/M * s] | [1/s] | [M] | Rmax | Chi^{∧}2 |
| IL4 | | | | | |
| IL13 | 2.44E+05 | 2.50E−05 | 1.03E−10 | 33 | 0.938 |
| TNFα | | | | | |
| Binding | 6.25E+05 | 9.27E−06 | 1.48E−11 | 22 | 0.176 |
| Protein 9 | | | | | |
| Binding | 7.16E+05 | 3.76E−05 | 5.25E−11 | 25 | 0.145 |
| Protein 10 | | | | | |
| Binding | 2.95E+05 | 4.28E−05 | 1.45E−10 | 16 | 0.372 |
| Protein 11 | | | | | |
| Binding | 4.17E+05 | 5.06E−05 | 1.21E−10 | 28 | 0.338 |
| Protein 12 | | | | | |
| Binding | 6.15E+05 | 7.58E−05 | 1.23E−10 | 23 | 0.186 |
| Protein 13 | | | | | |
| Binding | 6.93E+05 | 1.19E−04 | 1.72E−10 | 22 | 0.232 |
| Protein 14 | | | | | |
| Binding | 2.50E+05 | 5.61E−05 | 2.24E−10 | 30 | 0.631 |
| Protein 15 | | | | | |

TABLE H
summary of surface plasmon resonance results for TNFα
for various trispecific binding proteins

| | Ka | K_{d} | K_{D} | | |
|---|---|---|---|---|---|
| Construct | [1/M * s] | [1/s] | [M] | Rmax | Chi^{∧}2 |
| IL4 | | | | | |
| IL13 | | | | | |
| TNFα | 1.78E+05 | 1.64E−04 | 9.22E−10 | 33 | 0.745 |
| Binding | 1.32E+05 | 3.20E−04 | 2.42E−9 | 23 | 0.213 |
| Protein 9 | | | | | |
| Binding | 1.40E+05 | 2.90E−04 | 2.07E−9 | 27 | 0.241 |
| Protein 10 | | | | | |
| Binding | 3.36E+05 | 1.82E−04 | 5.41E−10 | 28 | 0.539 |
| Protein 11 | | | | | |
| Binding | 4.49E+05 | 1.80E−04 | 4.00E−10 | 28 | 0.647 |
| Protein 12 | | | | | |
| Binding | 5.84E+05 | 1.96E−04 | 3.35E−10 | 27 | 0.529 |
| Protein 13 | | | | | |
| Binding | 5.29E+05 | 1.86E−04 | 3.52E−10 | 25 | 0.485 |
| Protein 14 | | | | | |
| Binding | 5.59E+05 | 1.84E−04 | 3.28E−10 | 27 | 0.409 |
| Protein 15 | | | | | |

In order to assess the neutralization activity of the trispecific binding proteins, a cellular assay was performed using different HEK Blue kits (Invivogen). Cytokines were preincubated with different concentrations of anti-cytokine antibodies for 30 minutes at room temperatures in a 96 well plate. Controls included use of only the cytokine or only the antibody. 50,000 HEK Blue Cells (HEK Blue TNFα/IL1β cells (InvivoGen, Cat. #hkb-tnfil1; HEK Blue STAT-6 cells (InvivoGen, Cat. #Hkb.stat6) were added to the cytokine/antibody mixture and incubated for 23 hours at 37° C., 5% CO2 in an incubator. QuantiBlue Reagent was added to each culture well and incubated for 2 hours at 37° C. The OD was measured at 620 nm and the IC50 was calculated using BioStat Speed 2.0. The HEK Blue Reporter Cell Assay results of various trispecific antibodies are shown in Tables I and M.

Next, IC₅₀ values were calculated for Binding Proteins 9-15 and benchmarked against the single parental antibodies (Table I).

TABLE I
summary of HEK Blue Reporter Assays (IC₅₀ Data)
for various trispecific binding proteins

| | IL4 IC₅₀ | IL13 IC₅₀ | TNFα IC₅₀ |
|---|---|---|---|
| Construct | (ng/mL) | (ng/mL) | (ng/mL) |
| IL4 | 2.14E+00 | — | — |
| | 1.85E+00 | | |
| | 1.82E+01 | | |
| IL13 | — | 1.10E+02 | — |
| | | 8.83E+01 | |
| | | 1.42E+01 | |
| TNFα | — | — | 3.63E+00 |
| | | | 5.78E+00 |
| | | | 2.41E+00 |
| Binding | 4.51E+00 | 1.77E+02 | 3.95E+01 |
| Protein 9 | | | |
| Binding | 5.93E+00 | 4.68E+02 | 4.76E+01 |
| Protein 10 | | | |
| Binding | 6.96E+00 | 4.89E+02 | 2.65E+01 |
| Protein 11 | | | |
| Binding | 5.03E+00 | 1.83E+02 | 2.17E+01 |
| Protein 12 | | | |
| Binding | 1.38E+01 | 7.54E+01 | 2.26E+01 |
| Protein 13 | | | |
| Binding | 1.02E+01 | 1.20E+02 | 6.26E+00 |
| Protein 14 | | | |
| Binding | 1.30E+01 | 1.07E+02 | 2.38E+01 |
| Protein 15 | | | |

The thermostability of the bispecific-trivalent binding proteins was measured by differential scanning fluorimetry (DSF; Table J).

TABLE J
summary of the thermostability by DSF for various
trivalent binding proteins

| | Tm1 | Tm2 |
|---|---|---|
| Construct | (° C.) | (° C.) |
| IL4 | 70 | 81 |
| IL13 | 67 | 78 |
| Binding | 63 | — |
| Protein 16 | | |
| Binding | 63 | — |
| Protein 17 | | |
| Binding | 65 | — |
| Protein 18 | | |
| Binding | 55 | — |
| Protein 19 | | |

The binding affinity and number of target proteins bound by each of the bispecific-trivalent binding proteins was measured for human IL-4 (Table K) and IL-13 (Tables K and L).

TABLE K
summary of surface plasmon resonance results for IL-4 for various trivalent
binding proteins

| | | | | | | | | | No. of |
|---|---|---|---|---|---|---|---|---|---|
| | RU | | | | Rmax | | | | ILs |
| Construct | Capture | Analyte | Ka (1/Ms) | Kd (1/s) | (RU) | KD (M) | Chi2 | kDa Bound | Bound |
| IL4 | 116 | IL4 | 8.70E+07 | 1.57E−04 | 24 | 1.81E−12 | 0.240 | 15 | 1 |
| Binding | 218 | IL4 | 4.77E+07 | 2.80E−04 | 20 | 5.88E−12 | 0.172 | 17 | 1 |
| Protein 16 | | | | | | | | | |
| Binding | 218 | IL4 | 3.16E+08 | 7.60E−04 | 43 | 2.40E−12 | 0.278 | 35 | 2 |
| Protein 17 | | | | | | | | | |
| Binding | 215 | IL4 | 3.52E+07 | 3.59E−04 | 22 | 1.02E−12 | 0.408 | 18 | 1 |
| Protein 18 | | | | | | | | | |
| Binding | 226 | IL4 | 8.27E+07 | 3.85E−04 | 43 | 4.65E−12 | 0.486 | 34 | 2 |
| Protein 19 | | | | | | | | | |

TABLE L
summary of surface plasmon resonance results for IL-13 for various trivalent
binding proteins

| | | | | | | | | | No. of |
|---|---|---|---|---|---|---|---|---|---|
| | RU | | | | Rmax | | | | ILs |
| Construct | Capture | Analyte | Ka (1/Ms) | Kd (1/s) | (RU) | KD (M) | Chi2 | kDa Bound | Bound |
| IL13 | 201 | IL13 | 8.95E+05 | 5.47E−05 | 37 | 6.11E−11 | 0.211 | 14 | 1 |
| Binding | 226 | IL13 | 7.17E+05 | 4.54E−05 | 35 | 6.34E−11 | 0.132 | 26 | 2 |
| Protein 16 | | | | | | | | | |
| Binding | 235 | IL13 | 5.92E+05 | 5.70E−05 | 15 | 9.64E−11 | 0.128 | 11 | 1 |
| Protein 17 | | | | | | | | | |
| Binding | 231 | IL13 | 1.00E+06 | 3.54E−05 | 35 | 3.53E−11 | 0.166 | 28 | 2 |
| Protein 18 | | | | | | | | | |
| Binding | 282 | IL13 | 1.91E+06 | 3.81E−05 | 18 | 2.00E−11 | 0.265 | 14 | 1 |
| Protein 19 | | | | | | | | | |

Finally, IC₅₀ values were calculated for Binding Proteins 16-19 (Table M).

TABLE M
summary of HEK Blue Reporter Assays (IC₅₀
Data) for various trivalent binding proteins

| | IL4 IC₅₀ | IL13 IC₅₀ |
|---|---|---|
| Construct | (ng/mL) | (ng/mL) |
| IL4 | 6.07E+00 | — |
| IL13 | — | 1.12E+03 |
| Binding | 1.30E+01 | 1.24E+03 |
| Protein 16 | | |
| Binding | 8.62E+00 | 9.30E+03 |
| Protein 17 | | |
| Binding | 1.46E+01 | 1.10E+03 |
| Protein 18 | | |
| Binding | 5.73E+00 | 6.93E+03 |
| Protein 19 | | |

### Example 6: Trispecific Binding Protein Format Optimization

A problem with many existing heterodimeric binding protein formats (e.g., bispecific antibodies and variants thereof) is that it can be difficult to purify only the desired heterodimeric species without also including either homodimeric species. Thus, a process for efficient purification of the desired, heterodimeric binding protein is of great interest, e.g., for industrial-scale production.

As described herein, binding proteins of the present disclosure can include several optional features, including without limitation knob and hole mutations (e.g., to promote proper heterodimer formation) and mutations to improve purification. In addition, these binding proteins include two light chains, leading to four potential configurations: two kappa light chains, two lambda light chains, a kappa light chain on the arm with dual variable domains (the “CODV arm”) and a lambda light chain on the traditional antibody arm (the “Fab arm”), and a lambda light chain on the CODV arm and a kappa light chain on the Fab arm.

Therefore, experiments were undertaken to identify a process that allows for efficient purification of the desired binding protein of interest. Binding protein variants were also tested for their efficiency of purification.

FIG. 28A shows a diagram of an exemplary binding protein of the present disclosure, indicating variations that lead to unique configurations. These experiments tested the effect of the placement of: kappa and lambda light chains (e.g., two kappa, two lambda, kappa on CODV arm and lambda on Fab arm, and lambda on CODV arm and kappa on Fab arm), knob and hole mutations (e.g., knob mutations on CODV arm and hole mutations on Fab arm, or hole mutations on CODV arm and knob mutations on Fab arm), and H435R/Y436F mutations (“RF mutations,” e.g., RF mutations on CODV or Fab arm, or no RF mutations). A total of 18 different variants were tested, as shown in FIG. 28B. For these experiments, the CODV arm had antigen binding sites specific for TNFa (i.e., VH and VL sequences of SEQ ID NOs:168 and 169, respectively) and IL4 (i.e., VH and VL sequences of SEQ ID NOs:170 and 171, respectively), whereas the Fab arm had an antigen binding site specific for IL13 (i.e., VH and VL sequences of SEQ ID NOs:172 and 173, respectively). S354C and T366W were used for the knob mutations, and Y349C, T366S, L368A, and Y407V were used for the hole mutations.

Various processing steps were tested for the ability to monitor correct pairing of CODV and Fab arms (e.g., as opposed to CODV or Fab homodimers), as well as correct heavy chain-light chain pairing (e.g., as opposed to pairing between Fab arm light chain and CODV arm heavy chain, or between Fab arm heavy chain and CODV arm light chain). Analytical size exclusion chromatography (SEC) was found to be ineffective at distinguishing correct heavy chain and light chain pairing; binding proteins with Fab arm light chain mispaired with CODV heavy chain and homodimeric binding proteins with two Fab arms were found to co-elute with the desired trispecific binding proteins. However, analytical hydrophobic interaction chromatography (HIC) was found to resolve the desired trispecific binding proteins from binding proteins with Fab arm light chain mispaired with CODV heavy chain and homodimeric binding proteins with two Fab arms (FIG. 29).

The 18 binding protein configurations shown in FIG. 28B were purified by Protein A affinity chromatography, then KappaSelect (GE Healthcare) purification. Species were monitored by HIC chromatography. One binding protein configuration was purified efficiently without inclusion of mispaired species: lambda light chain for CODV arm, kappa light chain for Fab arm, knob mutations on CODV arm, hole mutations on Fab arm, and RF mutations on Fab arm. HIC chromatography (FIG. 30A), SDS-PAGE (FIG. 30B), and intact mass analysis demonstrated that a single species corresponding to the desired trispecific binding protein was purified.

These results identify a binding protein configuration that allows for more efficient purification of binding proteins of interest away from mispaired species. Moreover, the purification process of Protein A followed by KappaSelect purification steps was shown to provide effective separation of binding proteins of interest away from mispaired species.

While the disclosure includes various embodiments, it is understood that variations and modifications will occur to those skilled in the art. Therefore, it is intended that the appended claims cover all such equivalent variations that come within the scope of the disclosure. In addition, the section headings used herein are for organizational purposes only and are not to be construed as limiting the subject matter described.

Each embodiment herein described may be combined with any other embodiment or embodiments unless clearly indicated to the contrary. In particular, any feature or embodiment indicated as being preferred or advantageous may be combined with any other feature or features or embodiment or embodiments indicated as being preferred or advantageous, unless clearly indicated to the contrary.

All references cited in this application are expressly incorporated by reference herein.

### SEQUENCES

TABLE 1
Heavy and light chain SEQ ID NOs
for binding proteins 1-21 and the target
antigens to which the binding proteins are directed.

| Binding | SEQ ID | |
|---|---|---|
| Protein # | NOs Included | Directed to: |
| 1 | 1, 2, 3, 4 | Her2 × (CD28 × CD3) |
| 2 | 1, 2, 9, 10 | Her2 × (CD28 × CD3) |
| 3 | 13, 14, 3, 4 | CD19 × (CD28 × CD3) |
| 4 | 13, 14, 9, 10 | CD19 × (CD28 × CD3) |
| 5 | 17, 18, 3, 4 | CD38 × (CD28 × CD3) |
| 6 | 17, 18, 9, 10 | CD38 × (CD28 × CD3) |
| 7 | 21, 22, 3, 4 | LAMP1 × (CD28 × CD3) |
| 8 | 21, 22, 9, 10 | LAMP1 × (CD28 × CD3) |
| 9 | 60, 61, 62, 63 | TNFa × (IL4 × IL13) |
| 10 | 60, 61, 68, 69 | TNFa × (IL13 × IL4) |
| 11 | 60, 71, 68, 69 | TNFa × (IL13 × IL4) |
| 12 | 73, 74, 75, 76 | IL13 × (IL4 × TNFa) |
| 13 | 73, 74, 81, 82 | IL13 × (TNFa × IL4) |
| 14 | 85, 86, 87, 88 | IL4 × (IL13 × TNFa) |
| 15 | 85, 86, 93, 94 | IL4 × (TNFa × IL13) |
| 16 | 73, 74, 68, 69 | IL13 × (IL13 × IL4) |
| 17 | 85, 86, 68, 69 | IL4 × (IL13 × IL4) |
| 18 | 73, 74, 62, 63 | IL13 × (IL4 × IL13) |
| 19 | 85, 86, 62, 63 | IL4 × (IL4 × IL13) |
| 20 | 114, 115, 3, 4 | CD20 × (CD28 × CD3) |
| 21 | 114, 115, 9, 10 | CD20 × (CD28 × CD3) |

TABLE 2
Heavy and light chain sequences of binding proteins specifically directed to Her2,
CD3, CD28, CD19 and/or CD20. CDR sequences are bolded and italicized.
Binding Protein 1 Amino Acid Sequences

| | | |
|---|---|---|
| Heavy | Anti-Her2-H_Knob: | SEQ ID NO: 1 |
| chain A | Evqlvesggglvqpggsldscaas [symbol] ihwvrqapgkglewvar [symbol] | |
| (Anti- | ryadsvkgrftisadtskntaylqlmnslraedtavyyc [symbol] wgqgtl | |
| Her2- | vtvssastkgpsvfplapcsrstsestaalgclvkdyfpepvtvswnsgaltsgvhtfpa | |
| H_knob) | vlqssglyslssvvtvpssslgtktytcnvdhkpsntkvdkrveskygppcppcpape | |
| | flggpsvflfppkpkdtlmisrtpevtcvvvdvsqedpevqfnwyvdgvevhnakt | |
| | kpreeqfnstyrvvsvltvlhqdwlngkeykckvsnkglpssiektiskakgqprepq | |
| | vytlppcqeemtknqvslwclvkgfypsdiavewesngqpennykttppvldsdgs | |
| | fflyskltvdksrwqegnvfscsvmhealhnhytqkslslslgk | |
| Light | Anti-Her2-L: | SEQ ID NO: 2 |
| chain A | Diqmtqspsslsasvgdrvtitcras [symbol] vawyqqkpgkapklliysasflysgv | |
| (Anti- | psrfsgsrsgtdfdtisslqpedfatyy [symbol] fgqgtkveikrtvaapsyfifpp | |
| Her2-L) | sdeqlksgtasvvcllnnfypreakyqwkvdnalqsgnsqesvteqdskdstyslsstl | |
| | tlskadyekhkvyacevthqglsspvtksfnrgec | |
| Heavy | Anti-CD28 × Anti-CD3-H_Hole: | SEQ ID NO: 3 |
| chain B | Qvqlvqsgaevvkpgasvkvsckas [symbol] ihwvrqapgqglewigsi | |
| (Anti- | [symbol] nyaqkfqgratltvdtsistaymelsrlrsddtavyyc [symbol] | |
| CD28 × | [symbol] wgkgttvtvsssqvqlvesgggvvqpgrslrlscaas [symbol] mhw | |
| Anti-CD3- | vrqapgkqlewvaq [symbol] yatyyadsvkgrftisrddskntlylqmnslr | |
| H_Hole) | aedtavyyc [symbol] wgqgtlvtvssrtastkgpsvfplapcsrstses | |
| | taalgclvkdyfpepvtvswnsgaltsgvhtfpavlqssglyslssvvtvpsssl | |
| | gtktytcnvdhkpsntkvdkrveskygppcppcpapeflggpsvflfppkpk | |
| | dtlmisrtpevtcvvvdvsqedpevqfnwyvdgvevhnaktkpreeqfnst | |
| | yrvvsvltvlhqdwlngkeykckvsnkglpssiektiskakgqprepqvctlp | |
| | psqeemtknqvslscavkgfypsdiavewesngqpennykttppvldsdgs | |
| | fflvskltvdksrwqegnvfscsvmhealhnhytqkslslslgk | |
| Light | Anti-CD3 × Anti-CD28-L: | SEQ ID NO: 4 |
| chain B | Diymtqtplslsvtpgqpasisckss [symbol] lswylqkpgqspqsliy kvs n | |
| (Anti-CD3 × | rfsgvpdrfsgsgsgtdftlkisrveaedygyyyc [symbol] ftfgsgtkveikgqpka | |
| Anti- | apdiqmtqspsslsasygdrvtitcqas [symbol] lnwyqqkpgkapklliy [symbol] nlht | |
| CD28-L) | gvpsrfsgsgsgtdftltisslqpediatyyc [symbol] fgqgtkleiktkgpsrtvaa | |
| | psvfifppsdeqlksgtasvvcllnnfypreakyqwkydnalqsgnsqesvteqdsk | |
| | dstyslsstltlskadyekhkvyacevthqglsspytksfnrgec | |

Binding Protein 1 Nucleotide Sequences

| | | |
|---|---|---|
| Heavy | Anti-Her2-H_Knob: | SEQ ID NO: 5 |
| chain A | gaagtgcagctggtggaatctggcggcggactggtgcagcctggcggatctctgagact | |
| (Anti- | gagctgtgccgccagcggcttcaacatcaaggacacctacatccactgggtgcgccag | |
| Her2- | gcccctggcaagggactggaatgggtggccagaatctaccccaccaacggctacacca | |
| H_Knob:) | gatacgccgacagcgtgaagggccggttcaccatcagcgccgacaccagcaagaaca | |
| | ccgcctacctgcagatgaacagcctgcgggccgaggacaccgccgtgtactactgtagt | |
| | agatggggaggcgacggcttctacgccatggactattggggccagggcaccctcgtga | |
| | ccgtgtctagtgcgtcgaccaagggcccatcggtgttccctctggccccttgcagcagaa | |
| | gcaccagcgaatctacagccgccctgggctgcctcgtgaaggactactttcccgagccc | |
| | gtgaccgtgtcctggaactctggcgctctgacaagcggcgtgcacacctttccagccgtg | |
| | ctccagagcagcggcctgtactctctgagcagcgtcgtgacagtgcccagcagcagcct | |
| | gggcaccaagacctacacctgtaacgtggaccacaagcccagcaacaccaaggtgga | |
| | caagcgggtggaatctaagtacggccctccctgccctccttgcccagcccctgaatttctg | |
| | ggcggaccctccgtgttcctgttccccccaaagcccaaggacaccctgatgatcagccg | |
| | gacccccgaagtgacctgcgtggtggtggatgtgtcccaggaagatcccgaggtgcagt | |
| | tcaattggtacgtggacggcgtggaagtgcacaacgccaagaccaagcccagagagg | |
| | aacagttcaacagcacctaccgggtggtgtccgtgctgaccgtgctgcaccaggactgg | |
| | ctgaacggcaaagagtacaagtgcaaggtgtccaacaagggcctgcccagctccatcg | |
| | agaaaaccatcagcaaggccaagggccagccccgcgagcctcaagtgtataccctgcc | |
| | cccttgccaggaagagatgaccaagaaccaggtgtccctgtggtgtctcgtgaaaggctt | |
| | ctaccccagcgacattgccgtggaatgggagagcaacggccagcccgagaacaacta | |
| | caagaccaccccccctgtgctggacagcgacggctcattcttcctgtactccaagctgac | |
| | cgtggacaagagccggtggcaggaaggcaacgtgttcagctgctccgtgatgcacgag | |
| | gccctgcacaaccactacacccagaagtccctgtctctgtccctgggcaag | |
| Light | Anti-Her2-L: | SEQ ID NO: 6 |
| chain A | gacatccagatgacccagagccccagcagcctgtctgccagcgtgggcgacagagtga | |
| (Anti- | ccatcacctgtagagccagccaggacgtgaacaccgccgtggcctggtatcagcagaa | |
| Her2-L:) | gcctggcaaggcccccaagctgctgatctacagcgccagcttcctgtacagcggcgtgc | |
| | ccagcagattcagcggaagcagaagcggcaccgacttcaccctgaccatcagctccct | |
| | gcagcccgaggacttcgccacctactactgccagcagcactacaccaccccccccacat | |
| | ttggccagggcaccaaggtggaaatcaagcgtacggtggccgctcccagcgtgttcatc | |
| | ttcccacctagcgacgagcagctgaagtccggcacagcctctgtcgtgtgcctgctgaac | |
| | aacttctacccccgcgaggccaaagtgcagtggaaggtggacaacgccctgcagagcg | |
| | gcaacagccaggaaagcgtgaccgagcaggacagcaaggactccacctacagcctga | |
| | gcagcaccctgacactgagcaaggccgactacgagaagcacaaggtgtacgcctgcg | |
| | aagtgacccaccagggcctgtctagccccgtgaccaagagcttcaaccggggcgagtg | |
| | t | |
| Heavy | Anti-CD28 × Anti-CD3-H_Hole: | SEQ ID NO: 7 |
| chain B | caggtgcagctggtgcagtctggcgccgaggtcgtgaaacctggcgcctctgtgaaggt | |
| (Anti- | gtcctgcaaggccagcggctacacctaaccagctactacatccactgggtgcgccagg | |
| CD28 × | cccctggacagggactggaatggatcggcagcatctaccccggcaacgtgaacaccaa | |
| Anti-CD3- | ctacgcccagaagaccagggcagagccaccctgaccgtggacaccagcatcagcacc | |
| H_Hole:) | gcctacatggaactgagccggctgagaagcgacgacaccgccgtgtactactgcaccc | |
| | ggtcccactacggcctggattggaacttcgacgtgtggggcaagggcaccaccgtgac | |
| | agtgtctagcagccaggtgcagctggtggaatctggcggcggagtggtgcagcctggc | |
| | agaagcctgagactgagctgtgccgccagcggcttcaccttcaccaaggcctggatgca | |
| | ctgggtgcgccaggcccctggaaagcagctggaatgggtggcccagatcaaggacaa | |
| | gagcaacagctacgccacctactacgccgacagcgtgaagggccggttcaccatcagc | |
| | cgggacgacagcaagaacaccctgtacctgcagatgaacagcctgcgggccgaggac | |
| | accgccgtgtactactgtcggggcgtgtactatgccctgagccccttcgattactggggcc | |
| | agggaaccctcgtgaccgtgtctagtcggaccgccagcacaaagggcccatcggtgttc | |
| | cctctggccccttgcagcagaagcaccagcgaatctacagccgccctgggctgcctcgt | |
| | gaaggactactttcccgagcccgtgaccgtgtcctggaactctggcgctctgacaagcg | |
| | gcgtgcacaccttttccagccgtgctccagagcagcggcctgtactctctgagcagcgtcg | |
| | tgacagtgcccagcagcagcctgggcaccaagacctacacctgtaacgtggaccacaa | |
| | gcccagcaacaccaaggtggacaagcgggtggaatctaagtacggccctccctgccct | |
| | ccttgcccagcccctgaatactgggcggaccctccgtgacctgaccccccaaagccca | |
| | aggacaccctgatgatcagccggacccccgaagtgacctgcgtggtggtggatgtgtcc | |
| | caggaagatcccgaggtgcagttcaattggtacgtggacggcgtggaagtgcacaacg | |
| | ccaagaccaagcccagagaggaacagttcaacagcacctaccgggtggtgtccgtgct | |
| | gaccgtgctgcaccaggactggctgaacggcaaagagtacaagtgcaaggtgtccaac | |
| | aagggcctgcccagctccatcgagaaaaccatcagcaaggccaagggccagccccgc | |
| | gagcctcaagtgtgtaccctgccccctagccaggaagagatgaccaagaaccaggtgtc | |
| | cctgagctgtgccgtgaaaggcttctaccccagcgacattgccgtggaatgggagagca | |
| | acggccagcccgagaacaactacaagaccaccccccctgtgctggacagcgacggct | |
| | cattcttcctggtgtccaagctgaccgtggacaagagccggtggcaggaaggcaacgtg | |
| | ttcagctgctccgtgatgcacgaggccctgcacaaccactacacccagaagtccctgtct | |
| | ctgtccctgggcaag | |
| Light | Anti-CD3 × Anti-CD28-L: | SEQ ID NO: 8 |
| chain B | gacatcgtgatgacccagacccccctgagcctgagcgtgacacctggacagcctgcca | |
| (Anti-CD3 × | gcatcagctgcaagagcagccagagcctggtgcacaacaacgccaacacctacctgag | |
| Anti- | ctggtatctgcagaagcccggccagagcccccagtccctgatctacaaggtgtccaaca | |
| CD28-L:) | gattcagcggcgtgcccgacagattctccggcagcggctctggcaccgacttcaccctg | |
| | aagatcagccgggtggaagccgaggacgtgggcgtgtactattgtggccagggcaccc | |
| | agtaccccttcacctttggcagcggcaccaaggtggaaatcaagggccagcccaaggc | |
| | cgcccccgacatccagatgacccagagccccagcagcctgtctgccagcgtgggcga | |
| | cagagtgaccatcacctgtcaggccagccagaacatctacgtgtggctgaactggtatca | |
| | gcagaagcccggcaaggcccccaagctgctgatctacaaggccagcaacctgcacac | |
| | cggcgtgcccagcagattttctggcagcggctccggcaccgacttcaccctgacaatca | |
| | gctccctgcagcccgaggacattgccacctactactgccagcagggccagacctacccc | |
| | tacacctttggccagggcaccaagctggaaatcaagaccaagggccccagccgtacgg | |
| | tggccgctcccagcgtgttcatcttcccacctagcgacgagcagctgaagtccggcaca | |
| | gcctctgtcgtgtgcctgctgaacaacttctacccccgcgaggccaaagtgcagtggaag | |
| | gtggacaacgccctgcagagcggcaacagccaggaaagcgtgaccgagcaggacag | |
| | caaggactccacctacagcctgagcagcaccctgacactgagcaaggccgactacgag | |
| | aagcacaaggtgtacgcctgcgaagtgacccaccagggcctgtctagccccgtgacca | |
| | agagcttcaaccggggcgagtgt | |

Binding Protein 2 Amino Acid Sequences

| | | |
|---|---|---|
| Heavy | Anti-Her2-H_Knob: | SEQ ID NO: 1 |
| chain A | Evqlvesggglvqpggsldscaas [symbol] ihwvrqapgkglewvar [symbol] | |
| (Anti- | ryadsvkgrftisadtskntaylqlmnslraedtavyyc [symbol] wgqgtl | |
| Her2- | vtvssastkgpsvfplapcsrstsestaalgclvkdyfpepvtvswnsgaltsgvhtfpa | |
| H_knob) | vlqssglyslssvvtvpssslgtktytcnvdhkpsntkvdkrveskygppcppcpape | |
| | flggpsvflfppkpkdtlmisrtpevtcvvvdvsqedpevqfnwyvdgvevhnakt | |
| | kpreeqfnstyrvvsvltvlhqdwlngkeykckvsnkglpssiektiskakgqprepq | |
| | vytlppcqeemtknqvslwclvkgfypsdiavewesngqpennykttppvldsdgs | |
| | fflyskltvdksrwqegnvfscsvmhealhnhytqkslslslgk | |
| Light chain | Anti-Her2-L: | SEQ ID NO: 2 |
| A | Diqmtqspsslsasvgdrvtitcras [symbol] vawyqqkpgkapklliysasflysgv | |
| (Anti- | psrfsgsrsgtdfdtisslqpedfatyy [symbol] fgqgtkveikrtvaapsyfifpp | |
| Her2-L) | sdeqlksgtasvvcllnnfypreakyqwkvdnalqsgnsqesvteqdskdstyslsstl | |
| | tlskadyekhkvyacevthqglsspvtksfnrgec | |
| Heavy | Anti-CD28 × Anti-CD3-H_Hole | SEQ ID NO: 9 |
| chain B | Qvqlqesgpglvkpsqtlsltctvsgfslsdygvhwvrqppgkglewlgviw | |
| (Anti- | agggtnynpslksrktiskdtsknqvslklssvtaadtavyycardkgysyyys | |
| CD28 × | mdywgqgttvtvsssqvqlvesgggvvqpgrslrlscaasgftftkawmhw | |
| Anti-CD3- | vrqapgkqlewvaqikdksnsyatyyadsvkgrftisrddskntlylqmnslr | |
| H_Hole) | aedtavyycrgvyyalspfdywgqgtlvtvssrtastkgpsvfplapcsrstses | |
| | taalgclvkdyfpepvtvswnsgaltsgvhtfpavlqssglyslssvvtvpsssl | |
| | gtktytcnvdhkpsntkvdkrveskygppcppcpapeflggpsvflfppkpk | |
| | dtlmisrtpevtcvvvdvsqedpevqfnwyvdgvevhnaktkpreeqfnst | |
| | yrvvsvltvlhqdwlngkeykckvsnkglpssiektiskakgqprepqvctlp | |
| | psqeemtknqvslscavkgfypsdiavewesngqpennykttppvldsdgs | |
| | fflvskltvdksrwqegnvfscsvmhealhnhytqkslslslgk | |
| Light chain | Anti-CD3 × Anti-CD28-L: | SEQ ID NO: 10 |
| B | Divmtqtplslsvtpgqpasisckssqslyhnnantylswylqkpgqspqsliykvsn | |
| (Anti-CD3 × | rfsgvpdrfsgsgsgtdftlkisrveaedvgvyycgqgtqypftfgsgtkveikgqpka | |
| Anti- | apdivltqspaslavspgqratitcrasesveyyvtslmqwyqqkpgqppkllifaasn | |
| CD28-L) | vesgvparfsgsgsgtdftltinpveandvanyycqqsrkvpytfgqgtkleiktkgps | |
| | rtvaapsvfifppsdeqlksgtasyycllnnfypreakvqwkvdnalqsgnsqesvte | |
| | qdskdstyslsstltlskadyekhkvyacevthqglsspvtksfnrgec | |

Binding Protein 2 Nucleotide Sequences

| | | |
|---|---|---|
| Heavy | Anti-Her2-H_Knob: | SEQ ID NO: 5 |
| chain A | gaagtgcagctggtggaatctggcggcggactggtgcagcctggcggatctctgagact | |
| (Anti- | gagctgtgccgccagcggcttcaacatcaaggacacctacatccactgggtgcgccagg | |
| Her2- | cccctggcaagggactggaatgggtggccagaatctaccccaccaacggctacaccag | |
| H_Knob:) | atacgccgacagcgtgaagggccggttcaccatcagcgccgacaccagcaagaacac | |
| | cgcctacctgcagatgaacagcctgcgggccgaggacaccgccgtgtactactgtagta | |
| | gatggggaggcgacggcttctacgccatggactattggggccagggcaccctcgtgac | |
| | cgtgtctagtgcgtcgaccaagggcccatcggtgttccctctggccccttgcagcagaag | |
| | caccagcgaatctacagccgccctgggctgcctcgtgaaggactactttcccgagcccgt | |
| | gaccgtgtcctggaactctggcgctctgacaagcggcgtgcacacctttccagccgtgct | |
| | ccagagcagcggcctgtactctctgagcagcgtcgtgacagtgcccagcagcagcctg | |
| | ggcaccaagacctacacctgtaacgtggaccacaagcccagcaacaccaaggtggaca | |
| | agcgggtggaatctaagtacggccctccctgccctccttgcccagcccctgaatttctggg | |
| | cggaccctccgtgttcctgttccccccaaagcccaaggacaccctgatgatcagccggac | |
| | ccccgaagtgacctgcgtggtggtggatgtgtcccaggaagatcccgaggtgcagttca | |
| | attggtacgtggacggcgtggaagtgcacaacgccaagaccaagcccagagaggaac | |
| | agttcaacagcacctaccgggtggtgtccgtgctgaccgtgctgcaccaggactggctga | |
| | acggcaaagagtacaagtgcaaggtgtccaacaagggcctgcccagctccatcgagaa | |
| | aaccatcagcaaggccaagggccagccccgcgagcctcaagtgtataccctgccccctt | |
| | gccaggaagagatgaccaagaaccaggtgtccctgtggtgtctcgtgaaaggcnctacc | |
| | ccagcgacattgccgtggaatgggagagcaacggccagcccgagaacaactacaaga | |
| | ccaccccccctgtgctggacagcgacggctcattcttcctgtactccaagctgaccgtgga | |
| | caagagccggtggcaggaaggcaacgtgttcagctgctccgtgatgcacgaggccctg | |
| | cacaaccactacacccagaagtccctgtctctgtccctgggcaag | |
| Light | Anti-Her2-L: | SEQ ID NO: 6 |
| chain A | gacatccagatgacccagagccccagcagcctgtctgccagcgtgggcgacagagtga | |
| (Anti- | ccatcacctgtagagccagccaggacgtgaacaccgccgtggcctggtatcagcagaa | |
| Her2-L:) | gcctggcaaggcccccaagctgctgatctacagcgccagcttcctgtacagcggcgtgc | |
| | ccagcagattcagcggaagcagaagcggcaccgacttcaccctgaccatcagctccctg | |
| | cagcccgaggacttcgccacctactactgccagcagcactacaccaccccccccacattt | |
| | ggccagggcaccaaggtggaaatcaagcgtacggtggccgctcccagcgtgttcatctt | |
| | cccacctagcgacgagcagctgaagtccggcacagcctctgtcgtgtgcctgctgaaca | |
| | acttctacccccgcgaggccaaagtgcagtggaaggtggacaacgccctgcagagcgg | |
| | caacagccaggaaagcgtgaccgagcaggacagcaaggactccacctacagcctgag | |
| | cagcaccctgacactgagcaaggccgactacgagaagcacaaggtgtacgcctgcgaa | |
| | gtgacccaccagggcctgtctagccccgtgaccaagagcttcaaccggggcgagtgt | |
| Heavy | Anti-CD28 × Anti-CD3_Hole: | SEQ ID NO: 11 |
| chain B | caggtgcagctgcaggaatctggccctggcctcgtgaagcctagccagaccctgagcct | |
| (Anti- | gacctgtaccgtgtccggcttcagcctgagcgactacggcgtgcactgggtgcgccagc | |
| CD28 × | cacctggaaaaggcctggaatggctgggcgtgatctgggctggcggaggcaccaacta | |
| Anti-CD3- | caaccccagcctgaagtccagaaagaccatcagcaaggacaccagcaagaaccaggt | |
| H_Hole:) | gtccctgaagctgagcagcgtgacagccgccgataccgccgtgtactactgcgccagag | |
| | acaagggctacagctactactacagcatggactactggggccagggcaccaccgtgac | |
| | cgtgtcatcctctcaggtgcagctggtggaatctggcggcggagtggtgcagcctggca | |
| | gaagcctgagactgagctgtgccgccagcggcttcaccttcaccaaggcctggatgcac | |
| | tgggtgcgccaggcccctggaaagcagctggaatgggtggcccagatcaaggacaag | |
| | agcaacagctacgccacctactacgccgacagcgtgaagggccggttcaccatcagcc | |
| | gggacgacagcaagaacaccctgtacctgcagatgaacagcctgcgggccgaggaca | |
| | ccgccgtgtactactgtcggggcgtgtactatgccctgagccccttcgattactggggcca | |
| | gggaaccctcgtgaccgtgtctagtcggaccgcttcgaccaagggcccatcggtgttccc | |
| | tctggccccttgcagcagaagcaccagcgaatctacagccgccctgggctgcctcgtga | |
| | aggactactttcccgagcccgtgaccgtgtcctggaactctggcgctctgacaagcggcg | |
| | tgcacacctttccagccgtgctccagagcagcggcctgtactctctgagcagcgtcgtga | |
| | cagtgcccagcagcagcctgggcaccaagacctacacctgtaacgtggaccacaagcc | |
| | cagcaacaccaaggtggacaagcgggtggaatctaagtacggccctccctgccctcctt | |
| | gcccagcccctgaatttctgggcggaccctccgtgttcctgttccccccaaagcccaagg | |
| | acaccctgatgatcagccggacccccgaagtgacctgcgtggtggtggatgtgtcccag | |
| | gaagatcccgaggtgcagttcaattggtacgtggacggcgtggaagtgcacaacgccaa | |
| | gaccaagcccagagaggaacagttcaacagcacctaccgggtggtgtccgtgctgacc | |
| | gtgctgcaccaggactggctgaacggcaaagagtacaagtgcaaggtgtccaacaagg | |
| | gcctgcccagctccatcgagaaaaccatcagcaaggccaagggccagccccgcgagc | |
| | ctcaagtgtgtaccctgccccctagccaggaagagatgaccaagaaccaggtgtccctg | |
| | agctgtgccgtgaaaggcttctaccccagcgacattgccgtggaatgggagagcaacgg | |
| | ccagcccgagaacaactacaagaccaccccccctgtgctggacagcgacggctcattct | |
| | tcctggtgtccaagctgaccgtggacaagagccggtggcaggaaggcaacgtgttcagc | |
| | tgctccgtgatgcacgaggccctgcacaaccactacacccagaagtccctgtctctgtcc | |
| | ctgggcaag | |
| Light | Anti-CD3 × Anti-CD28-L: | SEQ ID NO: 12 |
| chain B | gacatcgtgatgacccagacccccctgagcctgagcgtgacacctggacagcctgcca | |
| (Anti-CD3 × | gcatcagctgcaagagcagccagagcctggtgcacaacaacgccaacacctacctgag | |
| Anti- | ctggtatctgcagaagcccggccagagcccccagtccctgatctacaaggtgtccaaca | |
| CD28-L | gattcagcggcgtgcccgacagattctccggcagcggctctggcaccgacttcaccctg | |
| | aagatcagccgggtggaagccgaggacgtgggcgtgtactattgtggccagggcaccc | |
| | agtaccccttcacctttggcagcggcaccaaggtggaaatcaagggccagcccaaggc | |
| | cgcccccgacatcgtgctgacacagagccctgctagcctggccgtgtctcctggacaga | |
| | gggccaccatcacctgtagagccagcgagagcgtggaatattacgtgaccagcctgatg | |
| | cagtggtatcagcagaagcccggccagccccccaagctgctgattttcgccgccagcaa | |
| | cgtggaaagcggcgtgccagccagattttccggcagcggctctggcaccgacttcaccc | |
| | tgaccatcaaccccgtggaagccaacgacgtggccaactactactgccagcagagccg | |
| | gaaggtgccctacacctttggccagggcaccaagctggaaatcaagaccaagggcccc | |
| | agccgtacggtggccgctcccagcgtgttcatcttcccacctagcgacgagcagctgaa | |
| | gtccggcacagcctctgtcgtgtgcctgctgaacaacttctacccccgcgaggccaaagt | |
| | gcagtggaaggtggacaacgccctgcagagcggcaacagccaggaaagcgtgaccg | |
| | agcaggacagcaaggactccacctacagcctgagcagcaccctgacactgagcaagg | |
| | ccgactacgagaagcacaaggtgtacgcctgcgaagtgacccaccagggcctgtctag | |
| | ccccgtgaccaagagcttcaaccggggcgagtgt | |

Binding Protein 3 Amino Acid Sequences

| | | |
|---|---|---|
| Heavy | Anti-CD19(B34)-H_Knob: | SEQ ID NO: 13 |
| chain A | Qvqlvqsgaevkkpgssvkvsckasgyafssywmnwyrqapgqglewigqiwp | |
| (Anti- | gdgdtnynqkfkgratltadeststaymelsslrsedtavyycarretttvgryyyamdy | |
| CD19(B34)- | wgqgttvtvssastkgpsvfplapcsrstsestaalgclvkdyfpepvtvswnsgaltsg | |
| H_knob) | vhtfpavlqssglyslssvvtvpssslgtktytcnvdhkpsntkvdkrveskygppcpp | |
| | cpapeflggpsvflfppkpkdtlmisrtpevtcvvvdvsqedpevqfnwyvdgvev | |
| | hnaktkpreeqfnstyrvvsvltvlhqdwlngkeykckvsnkglpssiektiskakgq | |
| | prepqvytlppcqeemtknqvslwclvkgfypsdiavewesngqpennykttppvl | |
| | dsdgsfflyskltvdksrwqegnvfscsvmhealhnhytqkslslslgk | |
| Light | Anti-CD19(B34)-L: | SEQ ID NO: 14 |
| chain A | Dlvltqspaslavspgqratitckasqsvdydgdsylnwyqqkpgqppklliydasnl | |
| (Anti- | vsgvparfsgsgsgtdftltinpveandtanyycqqstedpwtfgqgtkleikrtvaaps | |
| CD19(B34)- | vfifppsdeqlksgtasvvcllnnfypreakvqwkvdnalqsgnsqesvteqdskdst | |
| L) | yslsstltlskadyekhkvyacevthqglsspvtksfnrgec | |
| Heavy | Anti-CD28 × Anti-CD3-H_Hole: | SEQ ID NO: 3 |
| chain B | Qvqlvqsgaevvkpgasvkvsckas [symbol] ihwvrqapgqglewigs [symbol] | |
| (Anti- | [symbol] nyaqkfqgratltvdtsistaymelsrlrsddtavyyc [symbol] | |
| CD28 × | [symbol] wgkgttvtvsssqvqlvesgggvvqpgrslrlscaas [symbol] mhwvr | |
| Anti-CD3- | qapgkqlewvaq [symbol] yatyyadsvkgrftisrddskntlylqmnslrae | |
| H_Hole) | dtavyyc [symbol] wgqgtlvtvssrtastkgpsvfplapcsrstsestaa | |
| | lgclvkdyfpepvtvswnsgaltsgvhtfpavlqssglyslssvvtvpssslgtkt | |
| | ytcnvdhkpsntkvdkrveskygppcppcpapeflggpsvflfppkpkdtl | |
| | misrtpevtcvvvdvsqedpevqfnwyvdgvevhnaktkpreeqfnstyrv | |
| | vsvltvlhqdwlngkeykckvsnkglpssiektiskakgqprepqvctlppsq | |
| | eemtknqvslscavkgfypsdiavewesngqpennykttppvldsdgsfflv | |
| | skltvdksrwqegnvfscsvmhealhnhytqkslslslgk | |
| Light | Anti-CD3 × Anti-CD28-L: | SEQ ID NO: 4 |
| chain B | Divmtqtplslsvtpgqpasisckss [symbol] lswylqkpgqspqsliy [symbol] sn | |
| (Anti-CD3 × | rfsgvpdrfsgsgsgtdftlkisrveaedvgvyyc [symbol] ftfgsgtkveikgqpka | |
| Anti- | apdiqmtqspsslsasvgdrvtitcqas [symbol] lnwyqqkpgkapklliy [symbol] nlht | |
| CD28-L) | gvpsrfsgsgsgtdftltisslqpediatyyc [symbol] fgqgtkleiktkgpsrtvaap | |
| | svfifppsdeqlksgtasvvcllnnfypreakvqwkvdnalqsgnsqesvteqdskds | |
| | tyslsstltlskadyekhkvyacevthqglsspvtksfnrgec | |

Binding Protein 3 Nucleotide Sequences

| | | |
|---|---|---|
| Heavy | Anti-CD19(B34)-H_Knob: | SEQ ID NO: 15 |
| chain A | caggtgcagctggtgcagagcggcgccgaagtgaagaagcctggcagcagcgtgaag | |
| (Anti- | gtgagctgcaaggccagcggctatgccttcagcagctactggatgaactgggtgaggca | |
| CD19(B34)- | ggcacctggccagggcctggagtggataggccaaatatggcctggcgatggcgacacc | |
| H_Knob) | aactacaaccagaagttcaagggcagagcgaccttgaccgccgacgagagcaccagc | |
| | accgcgtacatggagctgagcagcctgaggagcgaggacaccgccgtgtactattgcg | |
| | ccagaagggagaccaccaccgtgggcaggtactactacgccatggactactggggcca | |
| | gggaaccaccgtgaccgtgagcagcgcctcgaccaagggcccatcggtgttccctctg | |
| | gccccttgcagcagaagcaccagcgaatctacagccgccctgggctgcctcgtgaagg | |
| | actactttcccgagcccgtgaccgtgtcctggaactctggcgctctgacaagcggcgtgc | |
| | acacctttccagccgtgctccagagcagcggcctgtactctctgagcagcgtcgtgacag | |
| | tgcccagcagcagcctgggcaccaagacctacacctgtaacgtggaccacaagcccag | |
| | caacaccaaggtggacaagcgggtggaatctaagtacggccctccctgccctccttgcc | |
| | cagcccctgaatttctgggcggaccctccgtgttcctgttccccccaaagcccaaggaca | |
| | ccctgatgatcagccggacccccgaagtgacctgcgtggtggtggatgtgtcccaggaa | |
| | gatcccgaggtgcagttcaattggtacgtggacggcgtggaagtgcacaacgccaagac | |
| | caagcccagagaggaacagttcaacagcacctaccgggtggtgtccgtgctgaccgtgc | |
| | tgcaccaggactggctgaacggcaaagagtacaagtgcaaggtgtccaacaagggcct | |
| | gcccagctccatcgagaaaaccatcagcaaggccaagggccagccccgcgagcctca | |
| | agtgtataccctgcccccttgccaggaagagatgaccaagaaccaggtgtccctgtggtg | |
| | tctcgtgaaaggcttctaccccagcgacattgccgtggaatgggagagcaacggccagc | |
| | ccgagaacaactacaagaccaccccccctgtgctggacagcgacggctcattcttcctgt | |
| | actccaagctgaccgtggacaagagccggtggcaggaaggcaacgtgttcagctgctc | |
| | cgtgatgcacgaggccctgcacaaccactacacccagaagtccctgtctctgtccctggg | |
| | caag | |
| Light | Anti-CD19(B34)-L: | SEQ ID NO: 16 |
| chain A | gacctcgtgctgacccagagccctgcgagcctggctgtgagccctggccagagagcca | |
| (Anti- | ccatcacctgcaaagccagccagagcgtggactacgacggcgacagctacctcaactg | |
| CD19(B34)- | gtaccagcagaagcctggccagccccccaagctgctgatttacgatgccagcaacctgg | |
| L) | tgagcggcgtgcctgctagattcagcggctccggcagcggcaccgacttcaccctgacc | |
| | atcaaccccgtggaggccaacgacaccgccaactactactgccagcagagcacggag | |
| | gacccctggaccttcggccagggcacaaagctggagatcaagcgtacggtggccgctc | |
| | ccagcgtgttcatcacccacctagcgacgagcagctgaagtccggcacagcctctgtcg | |
| | tgtgcctgctgaacaacttctacccccgcgaggccaaagtgcagtggaaggtggacaac | |
| | gccctgcagagcggcaacagccaggaaagcgtgaccgagcaggacagcaaggactc | |
| | cacctacagcctgagcagcaccctgacactgagcaaggccgactacgagaagcacaag | |
| | gtgtacgcctgcgaagtgacccaccagggcctgtctagccccgtgaccaagagcttcaa | |
| | ccggggcgagtgt | |
| Heavy | Anti-CD28 × Anti-CD3-H_Hole: | SEQ ID NO: 7 |
| chain B | caggtgcagctggtgcagtctggcgccgaggtcgtgaaacctggcgcctctgtgaaggt | |
| (Anti- | gtcctgcaaggccagcggctacacctaaccagctactacatccactgggtgcgccaggc | |
| CD28 × | ccctggacagggactggaatggatcggcagcatctaccccggcaacgtgaacaccaac | |
| Anti-CD3- | tacgcccagaagttccagggcagagccaccctgaccgtggacaccagcatcagcaccg | |
| H_Hole) | cctacatggaactgagccggctgagaagcgacgacaccgccgtgtactactgcacccg | |
| | gtcccactacggcctggattggaacttcgacgtgtggggcaagggcaccaccgtgacag | |
| | tgtctagcagccaggtgcagctggtggaatctggcggcggagtggtgcagcctggcag | |
| | aagcctgagactgagctgtgccgccagcggcttcaccttcaccaaggcctggatgcact | |
| | gggtgcgccaggcccctggaaagcagctggaatgggtggcccagatcaaggacaaga | |
| | gcaacagctacgccacctactacgccgacagcgtgaagggccggttcaccatcagccg | |
| | ggacgacagcaagaacaccctgtacctgcagatgaacagcctgcgggccgaggacac | |
| | cgccgtgtactactgtcggggcgtgtactatgccctgagccccttcgattactggggccag | |
| | ggaaccctcgtgaccgtgtctagtcggaccgccagcacaaagggcccatcggtgaccc | |
| | tctggccccttgcagcagaagcaccagcgaatctacagccgccctgggctgcctcgtga | |
| | aggactactttcccgagcccgtgaccgtgtcctggaactctggcgctctgacaagcggcg | |
| | tgcacacctttccagccgtgctccagagcagcggcctgtactctctgagcagcgtcgtga | |
| | cagtgcccagcagcagcctgggcaccaagacctacacctgtaacgtggaccacaagcc | |
| | cagcaacaccaaggtggacaagcgggtggaatctaagtacggccctccctgccctcctt | |
| | gcccagcccctgaatttctgggcggaccctccgtgacctgaccccccaaagcccaagg | |
| | acaccctgatgatcagccggacccccgaagtgacctgcgtggtggtggatgtgtcccag | |
| | gaagatcccgaggtgcagttcaattggtacgtggacggcgtggaagtgcacaacgccaa | |
| | gaccaagcccagagaggaacagttcaacagcacctaccgggtggtgtccgtgctgacc | |
| | gtgctgcaccaggactggctgaacggcaaagagtacaagtgcaaggtgtccaacaagg | |
| | gcctgcccagctccatcgagaaaaccatcagcaaggccaagggccagccccgcgagc | |
| | ctcaagtgtgtaccctgccccctagccaggaagagatgaccaagaaccaggtgtccctg | |
| | agctgtgccgtgaaaggcttctaccccagcgacattgccgtggaatgggagagcaacgg | |
| | ccagcccgagaacaactacaagaccaccccccctgtgctggacagcgacggctcattct | |
| | tcctggtgtccaagctgaccgtggacaagagccggtggcaggaaggcaacgtgttcagc | |
| | tgctccgtgatgcacgaggccctgcacaaccactacacccagaagtccctgtctctgtcc | |
| | ctgggcaag | |
| Light | Anti-CD3 × Anti-CD28-L: | SEQ ID NO: 8 |
| chain B | gacatcgtgatgacccagacccccctgagcctgagcgtgacacctggacagcctgcca | |
| (Anti-CD3 × | gcatcagctgcaagagcagccagagcctggtgcacaacaacgccaacacctacctgag | |
| Anti- | ctggtatctgcagaagcccggccagagcccccagtccctgatctacaaggtgtccaaca | |
| CD28-L:) | gattcagcggcgtgcccgacagattctccggcagcggctctggcaccgacttcaccctg | |
| | aagatcagccgggtggaagccgaggacgtgggcgtgtactattgtggccagggcaccc | |
| | agtaccccttcacctttggcagcggcaccaaggtggaaatcaagggccagcccaaggc | |
| | cgcccccgacatccagatgacccagagccccagcagcctgtctgccagcgtgggcgac | |
| | agagtgaccatcacctgtcaggccagccagaacatctacgtgtggctgaactggtatcag | |
| | cagaagcccggcaaggcccccaagctgctgatctacaaggccagcaacctgcacaccg | |
| | gcgtgcccagcagattttctggcagcggctccggcaccgacttcaccctgacaatcagct | |
| | ccctgcagcccgaggacattgccacctactactgccagcagggccagacctacccctac | |
| | acctttggccagggcaccaagctggaaatcaagaccaagggccccagccgtacggtgg | |
| | ccgctcccagcgtgttcatcttcccacctagcgacgagcagctgaagtccggcacagcct | |
| | ctgtcgtgtgcctgctgaacaacttctacccccgcgaggccaaagtgcagtggaaggtgg | |
| | acaacgccctgcagagcggcaacagccaggaaagcgtgaccgagcaggacagcaag | |
| | gactccacctacagcctgagcagcaccctgacactgagcaaggccgactacgagaagc | |
| | acaaggtgtacgcctgcgaagtgacccaccagggcctgtctagccccgtgaccaagag | |
| | cttcaaccggggcgagtgt | |

Binding Protein 4 Amino Acid Sequences

| | | |
|---|---|---|
| Heavy | Anti-CD19(B34)-H_Knob: | SEQ ID NO: 13 |
| chain A | Qvqlvqsgaevkkpgssvkvsckasgyafssywmnwyrqapgqglewigqiwp | |
| (Anti- | gdgdtnynqkfkgratltadeststaymelsslrsedtavyycarretttvgryyyamdy | |
| CD19(B34)- | wgqgttvtvssastkgpsvfplapcsrstsestaalgclvkdyfpepvtvswnsgaltsg | |
| H_knob) | yhtfpavlqssglyslssvvtvpssslgtktytcnvdhkpsntkvdkrveskygppcpp | |
| | cpapeflggpsvflfppkpkdtlmisrtpevtcvvvdvsqedpevqfnwyvdgvev | |
| | hnaktkpreeqfnstyrvvsvltvlhqdwlngkeykckvsnkglpssiektiskakgq | |
| | prepqvytlppcqeemtknqvslwclvkgfypsdiavewesngqpennykttppvl | |
| | dsdgsfflyskltvdksrwqegnvfscsvmhealhnhytqkslslslgk | |
| Light | Anti-CD19(B34)-L: | SEQ ID NO: 14 |
| chain A | Dlvltqspaslavspgqratitckasqsvdydgdsylnwyqqkpgqppklliydasnl | |
| (Anti- | vsgvparfsgsgsgtdftltinpveandtanyycqqstedpwtfgqgtkleikrtvaaps | |
| CD19(B34)- | vfifppsdeqlksgtasvvcllnnfypreakvqwkvdnalqsgnsqesvteqdskdst | |
| L) | yslsstltlskadyekhkvyacevthqglsspvtksfnrgec | |
| Heavy | Anti-CD28 × Anti-CD3-H_Hole: | SEQ ID NO: 9 |
| chain B | Qvqlqesgpglvkpsqtlsltctvsgfslsdygvhwvrqppgkglewlgviw | |
| (Anti- | agggtnynpslksrktiskdtsknqvslklssvtaadtavyycardkgysyyys | |
| CD28 × | mdywgqgttvtvsssqvqlvesgggvvqpgrslrlscaasgftftkawmhw | |
| Anti-CD3- | vrqapgkqlewvaqikdksnsyatyyadsvkgrftisrddskntlylqmnslr | |
| H_Hole) | aedtavyycrgvyyalspfdywgqgtlvtvssrtastkgpsvfplapcsrstses | |
| | taalgclvkdyfpepvtvswnsgaltsgvhtfpavlqssglyslssvvtvpsssl | |
| | gtktytcnvdhkpsntkvdkrveskygppcppcpapeflggpsvflfppkpk | |
| | dtlmisrtpevtcvvvdvsqedpevqfnwyvdgvevhnaktkpreeqfnsty | |
| | rvvsvltvlhqdwlngkeykckvsnkglpssiektiskakgqprepqvctlpp | |
| | sqeemtknqvslscavkgfypsdiavewesngqpennykttppvldsdgsff | |
| | lvskltvdksrwqegnvfscsvmhealhnhytqkslslslgk | |
| Light | Anti-CD3 × Anti-CD28-L: | SEQ ID NO: 10 |
| chain B | Divmtqtplslsvtpgqpasisckssqslvhnnantylswylqkpgqspqsliykvsn | |
| (Anti-CD3 × | rfsgvpdrfsgsgsgtdftlkisrveaedvgvyycgqgtqypftfgsgtkveikgqpka | |
| Anti- | apdivltqspaslavspgqratitcrasesveyyvtslmqwyqqkpgqppkllifaasn | |
| CD28-L) | vesgvparfsgsgsgtdftltinpveandvanyycqqsrkvpytfgqgtkleiktkgps | |
| | rtvaapsvfifppsdeqlksgtasyycllnnfypreakvqwkvdnalqsgnsqesvte | |
| | qdskdstyslsstltlskadyekhkvyacevthqglsspvtksfnrgec | |

Binding Protein 4 Nucleotide Sequences

| | | |
|---|---|---|
| Heavy | Anti-CD19(B34)-H_Knob: | SEQ ID NO: 15 |
| chain A | caggtgcagctggtgcagagcggcgccgaagtgaagaagcctggcagcagcgtgaag | |
| (Anti- | gtgagctgcaaggccagcggctatgccttcagcagctactggatgaactgggtgaggca | |
| CD19(B34)- | ggcacctggccagggcctggagtggataggccaaatatggcctggcgatggcgacacc | |
| H_Knob) | aactacaaccagaagttcaagggcagagcgaccttgaccgccgacgagagcaccagc | |
| | accgcgtacatggagctgagcagcctgaggagcgaggacaccgccgtgtactattgcg | |
| | ccagaagggagaccaccaccgtgggcaggtactactacgccatggactactggggcca | |
| | gggaaccaccgtgaccgtgagcagcgcctcgaccaagggcccatcggtgaccctctg | |
| | gccccttgcagcagaagcaccagcgaatctacagccgccctgggctgcctcgtgaagg | |
| | actactttcccgagcccgtgaccgtgtcctggaactctggcgctctgacaagcggcgtgc | |
| | acacctttccagccgtgctccagagcagcggcctgtactctctgagcagcgtcgtgacag | |
| | tgcccagcagcagcctgggcaccaagacctacacctgtaacgtggaccacaagcccag | |
| | caacaccaaggtggacaagcgggtggaatctaagtacggccctccctgccctccttgcc | |
| | cagcccctgaatttctgggcggaccctccgtgacctgaccccccaaagcccaaggaca | |
| | ccctgatgatcagccggacccccgaagtgacctgcgtggtggtggatgtgtcccaggaa | |
| | gatcccgaggtgcagttcaattggtacgtggacggcgtggaagtgcacaacgccaagac | |
| | caagcccagagaggaacagttcaacagcacctaccgggtggtgtccgtgctgaccgtgc | |
| | tgcaccaggactggctgaacggcaaagagtacaagtgcaaggtgtccaacaagggcct | |
| | gcccagctccatcgagaaaaccatcagcaaggccaagggccagccccgcgagcctca | |
| | agtgtataccctgcccccttgccaggaagagatgaccaagaaccaggtgtccctgtggtg | |
| | tctcgtgaaaggcttctaccccagcgacattgccgtggaatgggagagcaacggccagc | |
| | ccgagaacaactacaagaccaccccccctgtgctggacagcgacggctcattcttcctgt | |
| | actccaagctgaccgtggacaagagccggtggcaggaaggcaacgtgttcagctgctc | |
| | cgtgatgcacgaggccctgcacaaccactacacccagaagtccctgtctctgtccctggg | |
| | caag | |
| Light | Anti-CD19(B34)-L: | SEQ ID NO: 16 |
| chain A | gacctcgtgctgacccagagccctgcgagcctggctgtgagccctggccagagagcca | |
| (Anti- | ccatcacctgcaaagccagccagagcgtggactacgacggcgacagctacctcaactg | |
| CD19(B34)- | gtaccagcagaagcctggccagccccccaagctgctgatttacgatgccagcaacctgg | |
| L) | tgagcggcgtgcctgctagattcagcggctccggcagcggcaccgacttcaccctgacc | |
| | atcaaccccgtggaggccaacgacaccgccaactactactgccagcagagcacggag | |
| | gacccctggaccttcggccagggcacaaagctggagatcaagcgtacggtggccgctc | |
| | ccagcgtgttcatcttcccacctagcgacgagcagctgaagtccggcacagcctctgtcg | |
| | tgtgcctgctgaacaacttctacccccgcgaggccaaagtgcagtggaaggtggacaac | |
| | gccctgcagagcggcaacagccaggaaagcgtgaccgagcaggacagcaaggactc | |
| | cacctacagcctgagcagcaccctgacactgagcaaggccgactacgagaagcacaag | |
| | gtgtacgcctgcgaagtgacccaccagggcctgtctagccccgtgaccaagagcttcaa | |
| | ccggggcgagtgt | |
| Heavy | Anti-CD28 × Anti-CD3-H_Hole: | SEQ ID NO: 11 |
| chain B | caggtgcagctgcaggaatctggccctggcctcgtgaagcctagccagaccctgagcct | |
| (Anti- | gacctgtaccgtgtccggcttcagcctgagcgactacggcgtgcactgggtgcgccagc | |
| CD28 × | cacctggaaaaggcctggaatggctgggcgtgatctgggctggcggaggcaccaacta | |
| Anti-CD3- | caaccccagcctgaagtccagaaagaccatcagcaaggacaccagcaagaaccaggt | |
| H_Hole:) | gtccctgaagctgagcagcgtgacagccgccgataccgccgtgtactactgcgccagag | |
| | acaagggctacagctactactacagcatggactactggggccagggcaccaccgtgac | |
| | cgtgtcatcctctcaggtgcagctggtggaatctggcggcggagtggtgcagcctggca | |
| | gaagcctgagactgagctgtgccgccagcggcttcaccttcaccaaggcctggatgcac | |
| | tgggtgcgccaggcccctggaaagcagctggaatgggtggcccagatcaaggacaag | |
| | agcaacagctacgccacctactacgccgacagcgtgaagggccggttcaccatcagcc | |
| | gggacgacagcaagaacaccctgtacctgcagatgaacagcctgcgggccgaggaca | |
| | ccgccgtgtactactgtcggggcgtgtactatgccctgagccccttcgattactggggcca | |
| | gggaaccctcgtgaccgtgtctagtcggaccgcttcgaccaagggcccatcggtgttccc | |
| | tctggccccttgcagcagaagcaccagcgaatctacagccgccctgggctgcctcgtga | |
| | aggactactttcccgagcccgtgaccgtgtcctggaactctggcgctctgacaagcggcg | |
| | tgcacacctttccagccgtgctccagagcagcggcctgtactctctgagcagcgtcgtga | |
| | cagtgcccagcagcagcctgggcaccaagacctacacctgtaacgtggaccacaagcc | |
| | cagcaacaccaaggtggacaagcgggtggaatctaagtacggccctccctgccctcctt | |
| | gcccagcccctgaatttctgggcggaccctccgtgttcctgttccccccaaagcccaagg | |
| | acaccctgatgatcagccggacccccgaagtgacctgcgtggtggtggatgtgtcccag | |
| | gaagatcccgaggtgcagttcaattggtacgtggacggcgtggaagtgcacaacgccaa | |
| | gaccaagcccagagaggaacagttcaacagcacctaccgggtggtgtccgtgctgacc | |
| | gtgctgcaccaggactggctgaacggcaaagagtacaagtgcaaggtgtccaacaagg | |
| | gcctgcccagctccatcgagaaaaccatcagcaaggccaagggccagccccgcgagc | |
| | ctcaagtgtgtaccctgccccctagccaggaagagatgaccaagaaccaggtgtccctg | |
| | agctgtgccgtgaaaggcttctaccccagcgacattgccgtggaatgggagagcaacgg | |
| | ccagcccgagaacaactacaagaccaccccccctgtgctggacagcgacggctcattct | |
| | tcctggtgtccaagctgaccgtggacaagagccggtggcaggaaggcaacgtgttcagc | |
| | tgctccgtgatgcacgaggccctgcacaaccactacacccagaagtccctgtctctgtcc | |
| | ctgggcaag | |
| Light | Anti-CD3 × Anti-CD28-L: | SEQ ID NO: 12 |
| chain B | gacatcgtgatgacccagacccccctgagcctgagcgtgacacctggacagcctgcca | |
| (Anti-CD3 × | gcatcagctgcaagagcagccagagcctggtgcacaacaacgccaacacctacctgag | |
| Anti- | ctggtatctgcagaagcccggccagagcccccagtccctgatctacaaggtgtccaaca | |
| CD28-L:) | gattcagcggcgtgcccgacagattctccggcagcggctctggcaccgacttcaccctg | |
| | aagatcagccgggtggaagccgaggacgtgggcgtgtactattgtggccagggcaccc | |
| | agtaccccttcacctttggcagcggcaccaaggtggaaatcaagggccagcccaaggc | |
| | cgcccccgacatcgtgctgacacagagccctgctagcctggccgtgtctcctggacaga | |
| | gggccaccatcacctgtagagccagcgagagcgtggaatattacgtgaccagcctgatg | |
| | cagtggtatcagcagaagcccggccagccccccaagctgctgattttcgccgccagcaa | |
| | cgtggaaagcggcgtgccagccagattttccggcagcggctctggcaccgacttcaccc | |
| | tgaccatcaaccccgtggaagccaacgacgtggccaactactactgccagcagagccg | |
| | gaaggtgccctacacctttggccagggcaccaagctggaaatcaagaccaagggcccc | |
| | agccgtacggtggccgctcccagcgtgttcatcttcccacctagcgacgagcagctgaa | |
| | gtccggcacagcctctgtcgtgtgcctgctgaacaacttctacccccgcgaggccaaagt | |
| | gcagtggaaggtggacaacgccctgcagagcggcaacagccaggaaagcgtgaccg | |
| | agcaggacagcaaggactccacctacagcctgagcagcaccctgacactgagcaagg | |
| | ccgactacgagaagcacaaggtgtacgcctgcgaagtgacccaccagggcctgtctag | |
| | ccccgtgaccaagagcttcaaccggggcgagtgt | |

Binding Protein 5 Amino Acid Sequences

| | | |
|---|---|---|
| Heavy | Anti-CD38-H_Knob: | SEQ ID NO: 17 |
| chain A | Qvqlvqsgaevakpgtsvklsckasgytftdywmqwykqrpgqglewigtiypgd | |
| (Anti- | gdtgyaqkfqgkatltadkssktvymhlsslasedsavyycargdyygsnsldywgq | |
| CD38- | gtsvtvssastkgpsvfplapcsrstsestaalgclvkdyfpepvtvswnsgaltsgvhtf | |
| H_knob) | pavlqssglyslssvvtvpssslgtktytcnvdhkpsntkvdkrveskygppcppcpa | |
| | peflggpsvflfppkpkdtlmisrtpevtcvvvdvsqedpevqfnwyvdgvevhna | |
| | ktkpreeqfnstyrvvsvltvlhqdwlngkeykckvsnkglpssiektiskakgqpre | |
| | pqvytlppcqeemtknqvslwclvkgfypsdiavewesngqpennykttppvldsd | |
| | gsfflyskltvdksrwqegnvfscsvmhealhnhytqkslslslgk | |
| Light chain | Anti-CD38-L: | SEQ ID NO: 18 |
| A | Divmtqshlsmstslgdpvsitckasqdvstvvawyqqkpgqsprrliysasyryig | |
| (Anti- | vpdrftgsgagtdftftissvqaedlavyycqqhysppytfgggtkleikrtvaapsyfif | |
| CD38-L) | ppsdeqlksgtasvvcllnnfypreakyqwkvdnalqsgnsqesvteqdskdstysls | |
| | stltlskadyekhkvyacevthqglsspvtksfnrgec | |
| Heavy | Anti-CD28 × Anti-CD3-H_Hole | SEQ ID NO: 3 |
| chain B | Qvqlvqsgaevvkpgasvkvsckas [symbol] ihwvrqapgqglewigsiy | |
| (Anti- | [symbol] nyaqkfqgratltvdtsistaymelsrlrsddtavyyc [symbol] | |
| CD28 × | [symbol] wgkgttvtvsssqvqlvesgggvvqpgrslrlscaas [symbol] mhwv | |
| Anti-CD3- | rqapgkqlewvaq [symbol] yatyyadsvkgrftisrddskntlylqmnslra | |
| H_Hole) | edtavyyc [symbol] wgqgtlvtvssrtastkgpsvfplapcsrstsest | |
| | aalgclvkdyfpepvtvswnsgaltsgvhtfpavlqssglyslssvvtvpssslg | |
| | tktytcnvdhkpsntkvdkrveskygppcppcpapeflggpsvflfppkpkd | |
| | tlmisrtpevtcvvvdvsqedpevqfnwyvdgvevhnaktkpreeqfnstyr | |
| | vvsvltvlhqdwlngkeykckvsnkglpssiektiskakgqprepqvctlpps | |
| | qeemtknqvslscavkgfypsdiavewesngqpennykttppvldsdgsffl | |
| | vskltvdksrwqegnvfscsvmhealhnhytqkslslslgk | |
| Light chain | Anti-CD3 × Anti-CD28-L: | SEQ ID NO: 4 |
| B | Divmtqtplslsvtpgqpasisckss [symbol] lswylqkpgqspqsliykvsn | |
| (Anti-CD3 × | rfsgvpdrfsgsgsgtdftlkisrveaedvgvyyc [symbol] ftfgsgtkveikgqpka | |
| Anti- | apdiqmtqspsslsasvgdrvtitcqas [symbol] lnwyqqkpgkapklliy [symbol] nlht | |
| CD28-L) | gvpsrfsgsgsgtdftltisslqpediatyyc [symbol] fgqgtkleiktkgpsrtvaap | |
| | svfifppsdeqlksgtasvvcllnnfypreakvqwkvdnalqsgnsqesvteqdskds | |
| | tyslsstltlskadyekhkvyacevthqglsspvtksfnrgec | |

Binding Protein 5 Nucleotide Sequences

| | | |
|---|---|---|
| Heavy | Anti-CD38-H_Knob: | SEQ ID NO: 19 |
| chain A | caggtgcagctggtgcagtctggcgccgaagtggccaagcctggcacaagcgtgaagc | |
| (Anti- | tgagctgcaaggccagcggctacaccttcaccgactactggatgcagtgggtcaagcag | |
| CD38- | aggccaggccagggcctggaatggatcggcacaatctatcccggcgacggcgatacc | |
| H_Knob) | ggctacgcccagaagtttcagggcaaggccaccctgaccgccgacaagagcagcaag | |
| | accgtgtacatgcacctgagcagcctggccagcgaggacagcgccgtgtactattgcgc | |
| | cagaggcgactactacggcagcaacagcctggactattggggccagggcaccagcgt | |
| | gacagtgtctagtgcgtcgaccaagggcccatcggtgttccctctggccccttgcagcag | |
| | aagcaccagcgaatctacagccgccctgggctgcctcgtgaaggactactttcccgagc | |
| | ccgtgaccgtgtcctggaactctggcgctctgacaagcggcgtgcacacctttccagccg | |
| | tgctccagagcagcggcctgtactctctgagcagcgtcgtgacagtgcccagcagcagc | |
| | ctgggcaccaagacctacacctgtaacgtggaccacaagcccagcaacaccaaggtgg | |
| | acaagcgggtggaatctaagtacggccctccctgccctccttgcccagcccctgaatttct | |
| | gggcggaccctccgtgttcctgttccccccaaagcccaaggacaccctgatgatcagcc | |
| | ggacccccgaagtgacctgcgtggtggtggatgtgtcccaggaagatcccgaggtgca | |
| | gttcaattggtacgtggacggcgtggaagtgcacaacgccaagaccaagcccagagag | |
| | gaacagttcaacagcacctaccgggtggtgtccgtgctgaccgtgctgcaccaggactg | |
| | gctgaacggcaaagagtacaagtgcaaggtgtccaacaagggcctgcccagctccatc | |
| | gagaaaaccatcagcaaggccaagggccagccccgcgagcctcaagtgtataccctgc | |
| | ccccttgccaggaagagatgaccaagaaccaggtgtccctgtggtgtctcgtgaaaggct | |
| | tctaccccagcgacattgccgtggaatgggagagcaacggccagcccgagaacaacta | |
| | caagaccaccccccctgtgctggacagcgacggctcattcttcctgtactccaagctgac | |
| | cgtggacaagagccggtggcaggaaggcaacgtgttcagctgctccgtgatgcacgag | |
| | gccctgcacaaccactacacccagaagtccctgtctctgtccctgggcaag | |
| Light chain | Anti-CD38-L: | SEQ ID NO: 20 |
| A | gacatcgtgatgacccagagccacctgagcatgagcaccagcctgggcgaccccgtgt | |
| (Anti- | ccatcacctgtaaagccagccaggacgtgtccaccgtggtggcctggtatcagcagaag | |
| CD38-L) | cctggccagagccccagacggctgatctacagcgccagctatcggtacatcggcgtgcc | |
| | cgacagattcaccggaagcggagccggcaccgacttcaccttcaccatcagctctgtgc | |
| | aggccgaggacctggccgtgtactactgccagcagcactacagccccccctacaccttt | |
| | ggcggaggcaccaagctggaaatcaagcgtacggtggccgctcccagcgtgttcatctt | |
| | cccacctagcgacgagcagctgaagtccggcacagcctctgtcgtgtgcctgctgaaca | |
| | acttctacccccgcgaggccaaagtgcagtggaaggtggacaacgccctgcagagcgg | |
| | caacagccaggaaagcgtgaccgagcaggacagcaaggactccacctacagcctgag | |
| | cagcaccctgacactgagcaaggccgactacgagaagcacaaggtgtacgcctgcgaa | |
| | gtgacccaccagggcctgtctagccccgtgaccaagagcttcaaccggggcgagtgt | |
| Heavy | Anti-CD28 × Anti-CD3-H_Hole: | SEQ ID NO: 7 |
| chain B | caggtgcagctggtgcagtctggcgccgaggtcgtgaaacctggcgcctctgtgaaggt | |
| (Anti- | gtcctgcaaggccagcggctacacctttaaccagctactacatccactgggtgcgccaggc | |
| CD28 × | ccctggacagggactggaatggatcggcagcatctaccccggcaacgtgaacaccaac | |
| Anti-CD3- | tacgcccagaagttccagggcagagccaccctgaccgtggacaccagcatcagcaccg | |
| H_Hole) | cctacatggaactgagccggctgagaagcgacgacaccgccgtgtactactgcacccg | |
| | gtcccactacggcctggattggaacttcgacgtgtggggcaagggcaccaccgtgacag | |
| | tgtctagcagccaggtgcagctggtggaatctggcggcggagtggtgcagcctggcag | |
| | aagcctgagactgagctgtgccgccagcggcttcaccttcaccaaggcctggatgcact | |
| | gggtgcgccaggcccctggaaagcagctggaatgggtggcccagatcaaggacaaga | |
| | gcaacagctacgccacctactacgccgacagcgtgaagggccggttcaccatcagccg | |
| | ggacgacagcaagaacaccctgtacctgcagatgaacagcctgcgggccgaggacac | |
| | cgccgtgtactactgtcggggcgtgtactatgccctgagccccttcgattactggggccag | |
| | ggaaccctcgtgaccgtgtctagtcggaccgccagcacaaagggcccatcggtgaccc | |
| | tctggccccttgcagcagaagcaccagcgaatctacagccgccctgggctgcctcgtga | |
| | aggactactttcccgagcccgtgaccgtgtcctggaactctggcgctctgacaagcggcg | |
| | tgcacacctttccagccgtgctccagagcagcggcctgtactctctgagcagcgtcgtga | |
| | cagtgcccagcagcagcctgggcaccaagacctacacctgtaacgtggaccacaagcc | |
| | cagcaacaccaaggtggacaagcgggtggaatctaagtacggccctccctgccctcctt | |
| | gcccagcccctgaatttctgggcggaccctccgtgacctgaccccccaaagcccaagg | |
| | acaccctgatgatcagccggacccccgaagtgacctgcgtggtggtggatgtgtcccag | |
| | gaagatcccgaggtgcagttcaattggtacgtggacggcgtggaagtgcacaacgccaa | |
| | gaccaagcccagagaggaacagttcaacagcacctaccgggtggtgtccgtgctgacc | |
| | gtgctgcaccaggactggctgaacggcaaagagtacaagtgcaaggtgtccaacaagg | |
| | gcctgcccagctccatcgagaaaaccatcagcaaggccaagggccagccccgcgagc | |
| | ctcaagtgtgtaccctgccccctagccaggaagagatgaccaagaaccaggtgtccctg | |
| | agctgtgccgtgaaaggcttctaccccagcgacattgccgtggaatgggagagcaacgg | |
| | ccagcccgagaacaactacaagaccaccccccctgtgctggacagcgacggctcattct | |
| | tcctggtgtccaagctgaccgtggacaagagccggtggcaggaaggcaacgtgttcag | |
| | ctgctccgtgatgcacgaggccctgcacaaccactacacccagaagtccctgtctctgtc | |
| | cctgggcaag | |
| Light chain | Anti-CD3 × Anti-CD28-L: | SEQ ID NO: 8 |
| B | gacatcgtgatgacccagacccccctgagcctgagcgtgacacctggacagcctgcca | |
| (Anti-CD3 × | gcatcagctgcaagagcagccagagcctggtgcacaacaacgccaacacctacctgag | |
| Anti- | ctggtatctgcagaagcccggccagagcccccagtccctgatctacaaggtgtccaaca | |
| CD28-L:) | gattcagcggcgtgcccgacagattctccggcagcggctctggcaccgacttcaccctg | |
| | aagatcagccgggtggaagccgaggacgtgggcgtgtactattgtggccagggcaccc | |
| | agtaccccttcacctttggcagcggcaccaaggtggaaatcaagggccagcccaaggc | |
| | cgcccccgacatccagatgacccagagccccagcagcctgtctgccagcgtgggcgac | |
| | agagtgaccatcacctgtcaggccagccagaacatctacgtgtggctgaactggtatcag | |
| | cagaagcccggcaaggcccccaagctgctgatctacaaggccagcaacctgcacacc | |
| | ggcgtgcccagcagattttctggcagcggctccggcaccgacttcaccctgacaatcag | |
| | ctccctgcagcccgaggacattgccacctactactgccagcagggccagacctacccct | |
| | acacctttggccagggcaccaagctggaaatcaagaccaagggccccagccgtacggt | |
| | ggccgctcccagcgtgttcatcttcccacctagcgacgagcagctgaagtccggcacag | |
| | cctctgtcgtgtgcctgctgaacaacttctacccccgcgaggccaaagtgcagtggaagg | |
| | tggacaacgccctgcagagcggcaacagccaggaaagcgtgaccgagcaggacagc | |
| | aaggactccacctacagcctgagcagcaccctgacactgagcaaggccgactacgaga | |
| | agcacaaggtgtacgcctgcgaagtgacccaccagggcctgtctagccccgtgaccaa | |
| | gagcttcaaccggggcgagtgt | |

Binding Protein 6 Amino Acid Sequences

| | | |
|---|---|---|
| Heavy | Anti-CD38-H_Knob: | SEQ ID NO: 17 |
| chain A | qvqlvqsgaevakpgtsvklsckasgytftdywmqwykqrpgqglewigtiypgd | |
| (Anti- | gdtgyaqkfqgkatltadkssktvymhlsslasedsavyycargdyygsnsldywgq | |
| CD38- | gtsvtvssastkgpsvfplapcsrstsestaalgclvkdyfpepvtvswnsgaltsgvhtf | |
| H_knob) | pavlqssglyslssvvtvpssslgtktytcnvdhkpsntkvdkrveskygppcppcpa | |
| | peflggpsvflfppkpkdtlmisrtpevtcvvvdvsqedpevqfnwyvdgvevhna | |
| | ktkpreeqfnstyrvvsvltvlhqdwlngkeykckvsnkglpssiektiskakgqpre | |
| | pqyytlppcqeemtknqvslwclvkgfypsdiavewesngqpennykttppvldsd | |
| | gsfflyskltvdksrwqegnvfscsvmhealhnhytqkslslslgk | |
| Light chain | Anti-CD38-L: | SEQ ID NO: 18 |
| A | Divmtqshlsmstslgdpvsitckasqdvstvvawyqqkpgqsprrliysasyryig | |
| (Anti- | vpdrftgsgagtdftftissvqaedlavyycqqhysppytfgggtkleikrtvaapsyfif | |
| CD38-L) | ppsdeqlksgtasvvcllnnfypreakyqwkvdnalqsgnsqesvteqdskdstysls | |
| | stltlskadyekhkvyacevthqglsspvtksfnrgec | |
| Heavy | Anti-CD28 × Anti-CD3-H_Hole | SEQ ID NO: 9 |
| chain B | Qvqlqesgpglvkpsqtlsltctvsgfslsdygvhwvrqppgkglewlgviw | |
| (Anti- | agggtnynpslksrktiskdtsknqvslklssvtaadtavyycardkgysyyys | |
| CD28 × | mdywgqgttvtvsssqvqlvesgggvvqpgrslrlscaasgftftkawmhw | |
| Anti-CD3- | vrqapgkqlewvaqikdksnsyatyyadsvkgrftisrddskntlylqmnslr | |
| H_Hole) | aedtavyycrgvyyalspfdywgqgtlvtvssrtastkgpsvfplapcsrstses | |
| | taalgclvkdyfpepvtvswnsgaltsgvhtfpavlqssglyslssvvtvpsssl | |
| | gtktytcnvdhkpsntkvdkrveskygppcppcpapeflggpsvflfppkpk | |
| | dtlmisrtpevtcvvvdvsqedpevqfnwyvdgvevhnaktkpreeqfnsty | |
| | rvvsvltvlhqdwlngkeykckvsnkglpssiektiskakgqprepqvctlpp | |
| | sqeemtknqvslscavkgfypsdiavewesngqpennykttppvldsdgsff | |
| | lvskltvdksrwqegnvfscsvmhealhnhytqkslslslgk | |
| Light chain | Anti-CD3 × Anti-CD28-L: | SEQ ID NO: 10 |
| B | Divmtqtplslsvtpgqpasisckssqslvhnnantylswylqkpgqspqsliykvsn | |
| (Anti-CD3 × | rfsgvpdrfsgsgsgtdftlkisrveaedvgvyycgqgtqypftfgsgtkveikgqpka | |
| Anti- | apdivltqspaslavspgqratitcrasesveyyvtslmqwyqqkpgqppkllifaasn | |
| CD28-L) | vesgvparfsgsgsgtdftltinpveandvanyycqqsrkvpytfgqgtkleiktkgps | |
| | rtvaapsvfifppsdeqlksgtasyycllnnfypreakvqwkvdnalqsgnsqesvte | |
| | qdskdstyslsstltlskadyekhkvyacevthqglsspvtksfnrgec | |

Binding Protein 6 Nucleotide Sequences

| | | |
|---|---|---|
| Heavy | Anti-CD38-H_Knob: | SEQ ID NO: 19 |
| chain A | caggtgcagctggtgcagtctggcgccgaagtggccaagcctggcacaagcgtgaagc | |
| (Anti- | tgagctgcaaggccagcggctacaccttcaccgactactggatgcagtgggtcaagcag | |
| CD38- | aggccaggccagggcctggaatggatcggcacaatctatcccggcgacggcgatacc | |
| H_Knob) | ggctacgcccagaagtttcagggcaaggccaccctgaccgccgacaagagcagcaag | |
| | accgtgtacatgcacctgagcagcctggccagcgaggacagcgccgtgtactattgcgc | |
| | cagaggcgactactacggcagcaacagcctggactattggggccagggcaccagcgt | |
| | gacagtgtctagtgcgtcgaccaagggcccatcggtgttccctctggccccttgcagcag | |
| | aagcaccagcgaatctacagccgccctgggctgcctcgtgaaggactactttcccgagc | |
| | ccgtgaccgtgtcctggaactctggcgctctgacaagcggcgtgcacacctttccagccg | |
| | tgctccagagcagcggcctgtactctctgagcagcgtcgtgacagtgcccagcagcagc | |
| | ctgggcaccaagacctacacctgtaacgtggaccacaagcccagcaacaccaaggtgg | |
| | acaagcgggtggaatctaagtacggccctccctgccctccttgcccagcccctgaatttct | |
| | gggcggaccctccgtgttcctgttccccccaaagcccaaggacaccctgatgatcagcc | |
| | ggacccccgaagtgacctgcgtggtggtggatgtgtcccaggaagatcccgaggtgca | |
| | gttcaattggtacgtggacggcgtggaagtgcacaacgccaagaccaagcccagagag | |
| | gaacagttcaacagcacctaccgggtggtgtccgtgctgaccgtgctgcaccaggactg | |
| | gctgaacggcaaagagtacaagtgcaaggtgtccaacaagggcctgcccagctccatc | |
| | gagaaaaccatcagcaaggccaagggccagccccgcgagcctcaagtgtataccctgc | |
| | ccccttgccaggaagagatgaccaagaaccaggtgtccctgtggtgtctcgtgaaaggct | |
| | tctaccccagcgacattgccgtggaatgggagagcaacggccagcccgagaacaacta | |
| | caagaccaccccccctgtgctggacagcgacggctcattcttcctgtactccaagctgac | |
| | cgtggacaagagccggtggcaggaaggcaacgtgttcagctgctccgtgatgcacgag | |
| | gccctgcacaaccactacacccagaagtccctgtctctgtccctgggcaag | |
| Light chain | Anti-CD38-L: | SEQ ID NO: 20 |
| A | gacatcgtgatgacccagagccacctgagcatgagcaccagcctgggcgaccccgtgt | |
| (Anti- | ccatcacctgtaaagccagccaggacgtgtccaccgtggtggcctggtatcagcagaag | |
| CD38-L) | cctggccagagccccagacggctgatctacagcgccagctatcggtacatcggcgtgcc | |
| | cgacagattcaccggaagcggagccggcaccgacttcaccttcaccatcagctctgtgc | |
| | aggccgaggacctggccgtgtactactgccagcagcactacagccccccctacaccttt | |
| | ggcggaggcaccaagctggaaatcaagcgtacggtggccgctcccagcgtgttcatctt | |
| | cccacctagcgacgagcagctgaagtccggcacagcctctgtcgtgtgcctgctgaaca | |
| | acttctacccccgcgaggccaaagtgcagtggaaggtggacaacgccctgcagagcgg | |
| | caacagccaggaaagcgtgaccgagcaggacagcaaggactccacctacagcctgag | |
| | cagcaccctgacactgagcaaggccgactacgagaagcacaaggtgtacgcctgcgaa | |
| | gtgacccaccagggcctgtctagccccgtgaccaagagcttcaaccggggcgagtgt | |
| Heavy | Anti-CD28 × Anti-CD3_Hole: | SEQ ID NO: 11 |
| chain B | caggtgcagctgcaggaatctggccctggcctcgtgaagcctagccagaccctgagcct | |
| (Anti- | gacctgtaccgtgtccggatcagcctgagcgactacggcgtgcactgggtgcgccagc | |
| CD28 × | cacctggaaaaggcctggaatggctgggcgtgatctgggctggcggaggcaccaacta | |
| Anti-CD3- | caaccccagcctgaagtccagaaagaccatcagcaaggacaccagcaagaaccaggt | |
| H_Hole:) | gtccctgaagctgagcagcgtgacagccgccgataccgccgtgtactactgcgccaga | |
| | gacaagggctacagctactactacagcatggactactggggccagggcaccaccgtga | |
| | ccgtgtcatcctctcaggtgcagctggtggaatctggcggcggagtggtgcagcctggc | |
| | agaagcctgagactgagctgtgccgccagcggcttcaccttcaccaaggcctggatgca | |
| | ctgggtgcgccaggcccctggaaagcagctggaatgggtggcccagatcaaggacaa | |
| | gagcaacagctacgccacctactacgccgacagcgtgaagggccggttcaccatcagc | |
| | cgggacgacagcaagaacaccctgtacctgcagatgaacagcctgcgggccgaggac | |
| | accgccgtgtactactgtcggggcgtgtactatgccctgagccccttcgattactggggcc | |
| | agggaaccctcgtgaccgtgtctagtcggaccgcttcgaccaagggcccatcggtgttcc | |
| | ctctggccccttgcagcagaagcaccagcgaatctacagccgccctgggctgcctcgtg | |
| | aaggactactttcccgagcccgtgaccgtgtcctggaactctggcgctctgacaagcggc | |
| | gtgcacacctttccagccgtgctccagagcagcggcctgtactctctgagcagcgtcgtg | |
| | acagtgcccagcagcagcctgggcaccaagacctacacctgtaacgtggaccacaagc | |
| | ccagcaacaccaaggtggacaagcgggtggaatctaagtacggccctccctgccctcct | |
| | tgcccagcccctgaatttctgggcggaccctccgtgttcctgttccccccaaagcccaag | |
| | gacaccctgatgatcagccggacccccgaagtgacctgcgtggtggtggatgtgtccca | |
| | ggaagatcccgaggtgcagttcaattggtacgtggacggcgtggaagtgcacaacgcc | |
| | aagaccaagcccagagaggaacagttcaacagcacctaccgggtggtgtccgtgctga | |
| | ccgtgctgcaccaggactggctgaacggcaaagagtacaagtgcaaggtgtccaacaa | |
| | gggcctgcccagctccatcgagaaaaccatcagcaaggccaagggccagccccgcga | |
| | gcctcaagtgtgtaccctgccccctagccaggaagagatgaccaagaaccaggtgtccc | |
| | tgagctgtgccgtgaaaggcttctaccccagcgacattgccgtggaatgggagagcaac | |
| | ggccagcccgagaacaactacaagaccaccccccctgtgctggacagcgacggctcat | |
| | tcttcctggtgtccaagctgaccgtggacaagagccggtggcaggaaggcaacgtgttc | |
| | agctgctccgtgatgcacgaggccctgcacaaccactacacccagaagtccctgtctctg | |
| | tccctgggcaag | |
| Light chain | Anti-CD3 × Anti-CD28-L: | SEQ ID NO: 12 |
| B | gacatcgtgatgacccagacccccctgagcctgagcgtgacacctggacagcctgcca | |
| (Anti-CD3 × | gcatcagctgcaagagcagccagagcctggtgcacaacaacgccaacacctacctgag | |
| Anti- | ctggtatctgcagaagcccggccagagcccccagtccctgatctacaaggtgtccaaca | |
| CD28-L | gattcagcggcgtgcccgacagattctccggcagcggctctggcaccgacttcaccctg | |
| | aagatcagccgggtggaagccgaggacgtgggcgtgtactattgtggccagggcaccc | |
| | agtaccccttcacctttggcagcggcaccaaggtggaaatcaagggccagcccaaggc | |
| | cgcccccgacatcgtgctgacacagagccctgctagcctggccgtgtctcctggacaga | |
| | gggccaccatcacctgtagagccagcgagagcgtggaatattacgtgaccagcctgatg | |
| | cagtggtatcagcagaagcccggccagccccccaagctgctgattttcgccgccagcaa | |
| | cgtggaaagcggcgtgccagccagattttccggcagcggctctggcaccgacttcaccc | |
| | tgaccatcaaccccgtggaagccaacgacgtggccaactactactgccagcagagccg | |
| | gaaggtgccctacacctttggccagggcaccaagctggaaatcaagaccaagggcccc | |
| | agccgtacggtggccgctcccagcgtgttcatcttcccacctagcgacgagcagctgaa | |
| | gtccggcacagcctctgtcgtgtgcctgctgaacaacttctacccccgcgaggccaaagt | |
| | gcagtggaaggtggacaacgccctgcagagcggcaacagccaggaaagcgtgaccg | |
| | agcaggacagcaaggactccacctacagcctgagcagcaccctgacactgagcaagg | |
| | ccgactacgagaagcacaaggtgtacgcctgcgaagtgacccaccagggcctgtctag | |
| | ccccgtgaccaagagcttcaaccggggcgagtgt | |

Binding Protein 7 Amino Acid Sequences:

| | | |
|---|---|---|
| Heavy | Anti-LAMP1-H_Knob: | SEQ ID NO: 21 |
| chain A | qvqlvqsgaevkkpgssvkvsckas [symbol] wvkkspgqglewig [symbol] | |
| (Anti- | [symbol] ysqkfqgkatltadtststtymelsslrsedtavyycvr [symbol] wgqgtl | |
| LAMP1- | vtvssastkgpsvfplapcsrstsestaalgclvkdyfpepvtvswnsgaltsgvhtfpa | |
| H_knob) | vlqssglyslssvvtvpssslgtktytcnvdhkpsntkvdkrveskygppcppcpapef | |
| | lggpsvflfppkpkdtlmisrtpevtcvvvdvsqedpevqfnwyvdgvevhnaktk | |
| | preeqfnstyrvvsvltvlhqdwlngkeykckvsnkglpssiektiskakgqprepqv | |
| | ytlppcqeemtknqvslwclvkgfypsdiavewesngqpennykttppvldsdgsff | |
| | lyskltvdksrwqegnvfscsvmhealhnhytqksls1slgk | |
| Light chain | Anti-LAMP1-L: | SEQ ID NO: 22 |
| A | Diqmtqspsslsasvgdrvtitc [symbol] wyqdkpgkaprllih [symbol] gv | |
| (Anti- | psrfsgsgsgrdytltisnlepedfatyyc [symbol] fgggtkveikrtvaapsvfifpp | |
| LAMP1-L) | sdeqlksgtasvvcllnnfypreakvqwkvdnalqsgnsqesvteqdskdstyslsstl | |
| | tlskadyekhkvyacevthqglsspvtksfnrgec | |
| Heavy | Anti-CD28 × Anti-CD3-H_Hole | SEQ ID NO: 3 |
| chain B | Qvqlvqsgaevvkpgasvkvsckas [symbol] ihwvrqapgqglewigs [symbol] | |
| (Anti- | [symbol] nyaqkfqgratltvdtsistaymelsrlrsddtavyyc [symbol] | |
| CD28 × | [symbol] wgkgttvtvsssqvqlvesgggvvqpgrslrlscaas [symbol] mhwvr | |
| Anti-CD3- | qapgkqlewvaq [symbol] yatyyadsvkgrftisrddskntlylqmnslrae | |
| H_Hole) | dtavyyc [symbol] wgqgtlvtvssrtastkgpsvfplapcsrstsestaa | |
| | lgclvkdyfpepvtvswnsgaltsgvhtfpavlqssglyslssvvtvpssslgtkt | |
| | ytcnvdhkpsntkvdkrveskygppcppcpapeflggpsvflfppkpkdtl | |
| | misrtpevtcvvvdvsqedpevqfnwyvdgvevhnaktkpreeqfnstyrv | |
| | vsvltvlhqdwlngkeykckvsnkglpssiektiskakgqprepqvctlppsq | |
| | eemtknqvslscavkgfypsdiavewesngqpennykttppvldsdgsfflv | |
| | skltvdksrwqegnvfscsvmhealhnhytqkslslslgk | |
| Light chain | Anti-CD3 × Anti-CD28-L: | SEQ ID NO: 4 |
| B | Divmtqtplslsvtpgqpasisckss [symbol] lswylqkpgqspqsliykvsn | |
| (Anti-CD3 × | rfsgvpdrfsgsgsgtdftlkisrveaedvgvyyc [symbol] ftfgsgtkveikgqpka | |
| Anti- | apdiqmtqspsslsasvgdrvtitcqas [symbol] lnwyqqkpgkapklliy [symbol] nlht | |
| CD28L) | gvpsrfsgsgsgtdftltisslqpediatyyc [symbol] fgqgtkleiktkgpsrtvaap | |
| | svfifppsdeqlksgtasvvcllnnfypreakvqwkvdnalqsgnsqesvteqdskds | |
| | tyslsstltlskadyekhkvyacevthqglsspvtksfnrgec | |

Binding Protein 7 Nucleotide Sequences

| | | |
|---|---|---|
| Heavy | Anti-LAMP1-H_Knob: | SEQ ID NO: 23 |
| chain A | caggtgcagctggtgcagtctggcgccgaagtgaagaaacccggcagcagcgtgaag | |
| (Anti- | gtgtcctgcaaggccagcggctacatcttcaccaactacaacatccactgggtcaagaag | |
| LAMP1- | tccccaggccagggcctggaatggatcggcgccatctatcccggaaacggcgacgccc | |
| H_Knob) | cttacagccagaagttccagggcaaggccaccctgaccgccgataccagcacctccacc | |
| | acctacatggaactgagcagcctgcggagcgaggacaccgccgtgtactattgcgtgcg | |
| | ggccaactgggatgtggccttcgcctattggggccagggcacactcgtgaccgtgtcctc | |
| | tgcgtcgaccaagggcccatcggtgttccctctggccccttgcagcagaagcaccagcg | |
| | aatctacagccgccctgggctgcctcgtgaaggactactttcccgagcccgtgaccgtgt | |
| | cctggaactctggcgctctgacaagcggcgtgcacacctttccagccgtgctccagagca | |
| | gcggcctgtactctctgagcagcgtcgtgacagtgcccagcagcagcctgggcaccaa | |
| | gacctacacctgtaacgtggaccacaagcccagcaacaccaaggtggacaagcgggtg | |
| | gaatctaagtacggccctccctgccctccttgcccagcccctgaatttctgggcggaccct | |
| | ccgtgttcctgttccccccaaagcccaaggacaccctgatgatcagccggacccccgaa | |
| | gtgacctgcgtggtggtggatgtgtcccaggaagatcccgaggtgcagttcaattggtac | |
| | gtggacggcgtggaagtgcacaacgccaagaccaagcccagagaggaacagttcaac | |
| | agcacctaccgggtggtgtccgtgctgaccgtgctgcaccaggactggctgaacggcaa | |
| | agagtacaagtgcaaggtgtccaacaagggcctgcccagctccatcgagaaaaccatca | |
| | gcaaggccaagggccagccccgcgagcctcaagtgtataccctgcccccttgccagga | |
| | agagatgaccaagaaccaggtgtccctgtggtgtctcgtgaaaggcttctaccccagcga | |
| | cattgccgtggaatgggagagcaacggccagcccgagaacaactacaagaccacccc | |
| | ccctgtgctggacagcgacggctcattcttcctgtactccaagctgaccgtggacaagag | |
| | ccggtggcaggaaggcaacgtgttcagctgctccgtgatgcacgaggccctgcacaac | |
| | cactacacccagaagtccctgtctctgtccctgggcaag | |
| Light chain | Anti-LAMP1-L: | SEQ ID NO: 24 |
| A | gacatccagatgacccagagccccagcagcctgtctgccagcgtgggcgacagagtga | |
| (Anti- | ccatcacatgcaaggccagccaggacatcgatcggtacatggcctggtatcaggacaag | |
| LAMP1-L) | cccggcaaggcccccagactgctgatccacgataccagcacactgcagagcggcgtgc | |
| | ccagcagattttccggctctggcagcggcagagactacaccctgaccatcagcaacctg | |
| | gaacccgaggacttcgccacctactactgcctgcagtacgacaacctgtggaccttcggc | |
| | ggaggcaccaaggtggaaatcaagcgtacggtggccgctcccagcgtgttcatcttccc | |
| | acctagcgacgagcagctgaagtccggcacagcctctgtcgtgtgcctgctgaacaactt | |
| | ctacccccgcgaggccaaagtgcagtggaaggtggacaacgccctgcagagcggcaa | |
| | cagccaggaaagcgtgaccgagcaggacagcaaggactccacctacagcctgagcag | |
| | caccctgacactgagcaaggccgactacgagaagcacaaggtgtacgcctgcgaagtg | |
| | acccaccagggcctgtctagccccgtgaccaagagcttcaaccggggcgagtgt | |
| Heavy | Anti-CD28 × Anti-CD3-H_Hole: | SEQ ID NO: 7 |
| chain | caggtgcagctggtgcagtctggcgccgaggtcgtgaaacctggcgcctctgtgaaggt | |
| B(Anti- | gtcctgcaaggccagcggctacacctttaccagctactacatccactgggtgcgccaggc | |
| CD28 × | ccctggacagggactggaatggatcggcagcatctaccccggcaacgtgaacaccaac | |
| Anti-CD3- | tacgcccagaagaccagggcagagccaccctgaccgtggacaccagcatcagcaccg | |
| H_Hole) | cctacatggaactgagccggctgagaagcgacgacaccgccgtgtactactgcacccg | |
| | gtcccactacggcctggattggaacttcgacgtgtggggcaagggcaccaccgtgacag | |
| | tgtctagcagccaggtgcagctggtggaatctggcggcggagtggtgcagcctggcag | |
| | aagcctgagactgagctgtgccgccagcggcttcaccttcaccaaggcctggatgcact | |
| | gggtgcgccaggcccctggaaagcagctggaatgggtggcccagatcaaggacaaga | |
| | gcaacagctacgccacctactacgccgacagcgtgaagggccggttcaccatcagccg | |
| | ggacgacagcaagaacaccctgtacctgcagatgaacagcctgcgggccgaggacac | |
| | cgccgtgtactactgtcggggcgtgtactatgccctgagccccttcgattactggggccag | |
| | ggaaccctcgtgaccgtgtctagtcggaccgccagcacaaagggcccatcggtgttccc | |
| | tctggccccttgcagcagaagcaccagcgaatctacagccgccctgggctgcctcgtga | |
| | aggactactttcccgagcccgtgaccgtgtcctggaactctggcgctctgacaagcggcg | |
| | tgcacacctttccagccgtgctccagagcagcggcctgtactctctgagcagcgtcgtga | |
| | cagtgcccagcagcagcctgggcaccaagacctacacctgtaacgtggaccacaagcc | |
| | cagcaacaccaaggtggacaagcgggtggaatctaagtacggccctccctgccctcctt | |
| | gcccagcccctgaatttctgggcggaccctccgtgacctgaccccccaaagcccaagg | |
| | acaccctgatgatcagccggacccccgaagtgacctgcgtggtggtggatgtgtcccag | |
| | gaagatcccgaggtgcagttcaattggtacgtggacggcgtggaagtgcacaacgccaa | |
| | gaccaagcccagagaggaacagttcaacagcacctaccgggtggtgtccgtgctgacc | |
| | gtgctgcaccaggactggctgaacggcaaagagtacaagtgcaaggtgtccaacaagg | |
| | gcctgcccagctccatcgagaaaaccatcagcaaggccaagggccagccccgcgagc | |
| | ctcaagtgtgtaccctgccccctagccaggaagagatgaccaagaaccaggtgtccctg | |
| | agctgtgccgtgaaaggcttctaccccagcgacattgccgtggaatgggagagcaacgg | |
| | ccagcccgagaacaactacaagaccaccccccctgtgctggacagcgacggctcattct | |
| | tcctggtgtccaagctgaccgtggacaagagccggtggcaggaaggcaacgtgttcagc | |
| | tgctccgtgatgcacgaggccctgcacaaccactacacccagaagtccctgtctctgtcc | |
| | ctgggcaag | |
| Light chain | Anti-CD3 × Anti-CD28-L: | SEQ ID NO: 8 |
| B | gacatcgtgatgacccagacccccctgagcctgagcgtgacacctggacagcctgcca | |
| (Anti-CD3 × | gcatcagctgcaagagcagccagagcctggtgcacaacaacgccaacacctacctgag | |
| Anti- | ctggtatctgcagaagcccggccagagcccccagtccctgatctacaaggtgtccaaca | |
| CD28-L | gattcagcggcgtgcccgacagattctccggcagcggctctggcaccgacttcaccctg | |
| | aagatcagccgggtggaagccgaggacgtgggcgtgtactattgtggccagggcaccc | |
| | agtaccccttcacctttggcagcggcaccaaggtggaaatcaagggccagcccaaggc | |
| | cgcccccgacatccagatgacccagagccccagcagcctgtctgccagcgtgggcgac | |
| | agagtgaccatcacctgtcaggccagccagaacatctacgtgtggctgaactggtatcag | |
| | cagaagcccggcaaggcccccaagctgctgatctacaaggccagcaacctgcacaccg | |
| | gcgtgcccagcagattttctggcagcggctccggcaccgacttcaccctgacaatcagct | |
| | ccctgcagcccgaggacattgccacctactactgccagcagggccagacctacccctac | |
| | acctttggccagggcaccaagctggaaatcaagaccaagggccccagccgtacggtgg | |
| | ccgctcccagcgtgttcatcttcccacctagcgacgagcagctgaagtccggcacagcct | |
| | ctgtcgtgtgcctgctgaacaacttctacccccgcgaggccaaagtgcagtggaaggtgg | |
| | acaacgccctgcagagcggcaacagccaggaaagcgtgaccgagcaggacagcaag | |
| | gactccacctacagcctgagcagcaccctgacactgagcaaggccgactacgagaagc | |
| | acaaggtgtacgcctgcgaagtgacccaccagggcctgtctagccccgtgaccaagag | |
| | cttcaaccggggcgagtgt | |

Binding Protein 8 Amino Acid Sequences

| | | |
|---|---|---|
| Heavy | Anti-LAMP1-H_Knob: | SEQ ID NO: 21 |
| chain A | qvqlvqsgaevkkpgssvkvsckas [symbol] wvkkspgqglewig [symbol] | |
| (Anti- | [symbol] ysqkfqgkatltadtststtymelsslrsedtavyycvr [symbol] wgqgtl | |
| LAMP1- | vtvssastkgpsvfplapcsrstsestaalgclvkdyfpepvtvswnsgaltsgvhtfpa | |
| H_knob) | vlqssglyslssvvtvpssslgtktytcnvdhkpsntkvdkrveskygppcppcpapef | |
| | lggpsvflfppkpkdtlmisrtpevtcvvvdvsqedpevqfnwyvdgvevhnaktk | |
| | preeqfnstyrvvsvltvlhqdwlngkeykckvsnkglpssiektiskakgqprepqv | |
| | ytlppcqeemtknqvslwclvkgfypsdiavewesngqpennykttppvldsdgsff | |
| | lyskltvdksrwqegnvfscsvmhealhnhytqksls1slgk | |
| Light chain | Anti-LAMP1-L: | SEQ ID NO: 22 |
| A | Diqmtqspsslsasvgdrvtitc [symbol] wyqdkpgkaprllih [symbol] gv | |
| (Anti- | psrfsgsgsgrdytltisnlepedfatyyc [symbol] fgggtkveikrtvaapsvfifpp | |
| LAMP1-L) | sdeqlksgtasvvcllnnfypreakvqwkvdnalqsgnsqesvteqdskdstyslsstl | |
| | tlskadyekhkvyacevthqglsspvtksfnrgec | |
| Heavy | Anti-CD28 × Anti-CD3-H_Hole | SEQ ID NO: 9 |
| chain B | Qvqlqesgpglvkpsqtlsltctvsgfslsdygvhwvrqppgkglewlgviw | |
| (Anti- | agggtnynpslksrktiskdtsknqvslklssvtaadtavyycardkgysyyys | |
| CD28 × | mdywgqgttvtvsssqvqlvesgggvvqpgrslrlscaasgftftkawmhw | |
| Anti-CD3- | vrqapgkqlewvaqikdksnsyatyyadsvkgrftisrddskntlylqmnslr | |
| H_Hole) | aedtavyycrgvyyalspfdywgqgtlvtvssrtastkgpsvfplapcsrstses | |
| | taalgclvkdyfpepvtvswnsgaltsgvhtfpavlqssglyslssvvtvpsssl | |
| | gtktytcnvdhkpsntkvdkrveskygppcppcpapeflggpsvflfppkpk | |
| | dtlmisrtpevtcvvvdvsqedpevqfnwyvdgvevhnaktkpreeqfnsty | |
| | rvvsvltvlhqdwlngkeykckvsnkglpssiektiskakgqprepqvctlpp | |
| | sqeemtknqvslscavkgfypsdiavewesngqpennykttppvldsdgsff | |
| | lvskltvdksrwqegnvfscsvmhealhnhytqkslslslgk | |
| Light chain | Anti-CD3 × Anti-CD28-L: | SEQ ID NO: 10 |
| B | Divmtqtplslsvtpgqpasisckssqslvhnnantylswylqkpgqspqsliykvsn | |
| (Anti-CD3 × | rfsgvpdrfsgsgsgtdftlkisrveaedvgvyycgqgtqypftfgsgtkveikgqpka | |
| Anti- | apdivltqspaslavspgqratitcrasesveyyvtslmqwyqqkpgqppkllifaasn | |
| CD28-L) | vesgvparfsgsgsgtdftltinpveandvanyycqqsrkvpytfgqgtkleiktkgpsr | |
| | tvaapsvfifppsdeqlksgtasyycllnnfypreakvqwkvdnalqsgnsqesvteq | |
| | dskdstyslsstltlskadyekhkvyacevthqglsspvtksfnrgec | |

Binding Protein 8 Nucleotide Sequences

| | | |
|---|---|---|
| Heavy | Anti-LAMP1-H_Knob: | SEQ ID NO: 23 |
| chain A | caggtgcagctggtgcagtctggcgccgaagtgaagaaacccggcagcagcgtgaag | |
| (Anti- | gtgtcctgcaaggccagcggctacatcttcaccaactacaacatccactgggtcaagaag | |
| LAMP1- | tccccaggccagggcctggaatggatcggcgccatctatcccggaaacggcgacgccc | |
| H_Knob) | cttacagccagaagttccagggcaaggccaccctgaccgccgataccagcacctccacc | |
| | acctacatggaactgagcagcctgcggagcgaggacaccgccgtgtactattgcgtgcg | |
| | ggccaactgggatgtggccttcgcctattggggccagggcacactcgtgaccgtgtcctc | |
| | tgcgtcgaccaagggcccatcggtgttccctctggccccttgcagcagaagcaccagcg | |
| | aatctacagccgccctgggctgcctcgtgaaggactactttcccgagcccgtgaccgtgt | |
| | cctggaactctggcgctctgacaagcggcgtgcacacctttccagccgtgctccagagca | |
| | gcggcctgtactctctgagcagcgtcgtgacagtgcccagcagcagcctgggcaccaa | |
| | gacctacacctgtaacgtggaccacaagcccagcaacaccaaggtggacaagcgggtg | |
| | gaatctaagtacggccctccctgccctccttgcccagcccctgaatttctgggcggaccct | |
| | ccgtgttcctgttccccccaaagcccaaggacaccctgatgatcagccggacccccgaa | |
| | gtgacctgcgtggtggtggatgtgtcccaggaagatcccgaggtgcagttcaattggtac | |
| | gtggacggcgtggaagtgcacaacgccaagaccaagcccagagaggaacagttcaac | |
| | agcacctaccgggtggtgtccgtgctgaccgtgctgcaccaggactggctgaacggcaa | |
| | agagtacaagtgcaaggtgtccaacaagggcctgcccagctccatcgagaaaaccatca | |
| | gcaaggccaagggccagccccgcgagcctcaagtgtataccctgcccccttgccagga | |
| | agagatgaccaagaaccaggtgtccctgtggtgtctcgtgaaaggcttctaccccagcga | |
| | cattgccgtggaatgggagagcaacggccagcccgagaacaactacaagaccacccc | |
| | ccctgtgctggacagcgacggctcattcttcctgtactccaagctgaccgtggacaagag | |
| | ccggtggcaggaaggcaacgtgttcagctgctccgtgatgcacgaggccctgcacaac | |
| | cactacacccagaagtccctgtctctgtccctgggcaag | |
| Light chain | Anti-LAMP1-L: | SEQ ID NO: 24 |
| A | gacatccagatgacccagagccccagcagcctgtctgccagcgtgggcgacagagtga | |
| (Anti- | ccatcacatgcaaggccagccaggacatcgatcggtacatggcctggtatcaggacaag | |
| LAMP1-L) | cccggcaaggcccccagactgctgatccacgataccagcacactgcagagcggcgtgc | |
| | ccagcagattttccggctctggcagcggcagagactacaccctgaccatcagcaacctg | |
| | gaacccgaggacttcgccacctactactgcctgcagtacgacaacctgtggaccttcggc | |
| | ggaggcaccaaggtggaaatcaagcgtacggtggccgctcccagcgtgttcatcttccc | |
| | acctagcgacgagcagctgaagtccggcacagcctctgtcgtgtgcctgctgaacaactt | |
| | ctacccccgcgaggccaaagtgcagtggaaggtggacaacgccctgcagagcggcaa | |
| | cagccaggaaagcgtgaccgagcaggacagcaaggactccacctacagcctgagcag | |
| | caccctgacactgagcaaggccgactacgagaagcacaaggtgtacgcctgcgaagtg | |
| | acccaccagggcctgtctagccccgtgaccaagagcttcaaccggggcgagtgt | |
| Heavy | Anti-CD28 × Anti-CD3-H_Hole: | SEQ ID NO: 11 |
| chain B | caggtgcagctgcaggaatctggccctggcctcgtgaagcctagccagaccctgagcct | |
| (Anti- | gacctgtaccgtgtccggcttcagcctgagcgactacggcgtgcactgggtgcgccagc | |
| CD28 × | cacctggaaaaggcctggaatggctgggcgtgatctgggctggcggaggcaccaacta | |
| Anti-CD3- | caaccccagcctgaagtccagaaagaccatcagcaaggacaccagcaagaaccaggt | |
| H_Hole:) | gtccctgaagctgagcagcgtgacagccgccgataccgccgtgtactactgcgccagag | |
| | acaagggctacagctactactacagcatggactactggggccagggcaccaccgtgac | |
| | cgtgtcatcctctcaggtgcagctggtggaatctggcggcggagtggtgcagcctggca | |
| | gaagcctgagactgagctgtgccgccagcggcttcaccttcaccaaggcctggatgcac | |
| | tgggtgcgccaggcccctggaaagcagctggaatgggtggcccagatcaaggacaag | |
| | agcaacagctacgccacctactacgccgacagcgtgaagggccggttcaccatcagcc | |
| | gggacgacagcaagaacaccctgtacctgcagatgaacagcctgcgggccgaggaca | |
| | ccgccgtgtactactgtcggggcgtgtactatgccctgagccccttcgattactggggcca | |
| | gggaaccctcgtgaccgtgtctagtcggaccgcttcgaccaagggcccatcggtgttccc | |
| | tctggccccttgcagcagaagcaccagcgaatctacagccgccctgggctgcctcgtga | |
| | aggactactttcccgagcccgtgaccgtgtcctggaactctggcgctctgacaagcggcg | |
| | tgcacacctttccagccgtgctccagagcagcggcctgtactctctgagcagcgtcgtga | |
| | cagtgcccagcagcagcctgggcaccaagacctacacctgtaacgtggaccacaagcc | |
| | cagcaacaccaaggtggacaagcgggtggaatctaagtacggccctccctgccctcctt | |
| | gcccagcccctgaatttctgggcggaccctccgtgttcctgttccccccaaagcccaagg | |
| | acaccctgatgatcagccggacccccgaagtgacctgcgtggtggtggatgtgtcccag | |
| | gaagatcccgaggtgcagttcaattggtacgtggacggcgtggaagtgcacaacgccaa | |
| | gaccaagcccagagaggaacagttcaacagcacctaccgggtggtgtccgtgctgacc | |
| | gtgctgcaccaggactggctgaacggcaaagagtacaagtgcaaggtgtccaacaagg | |
| | gcctgcccagctccatcgagaaaaccatcagcaaggccaagggccagccccgcgagc | |
| | ctcaagtgtgtaccctgccccctagccaggaagagatgaccaagaaccaggtgtccctg | |
| | agctgtgccgtgaaaggcttctaccccagcgacattgccgtggaatgggagagcaacgg | |
| | ccagcccgagaacaactacaagaccaccccccctgtgctggacagcgacggctcattct | |
| | tcctggtgtccaagctgaccgtggacaagagccggtggcaggaaggcaacgtgttcagc | |
| | tgctccgtgatgcacgaggccctgcacaaccactacacccagaagtccctgtctctgtcc | |
| | ctgggcaag | |
| Light chain | Anti-CD3 × Anti-CD28-L: | SEQ ID NO: 12 |
| B | gacatcgtgatgacccagacccccctgagcctgagcgtgacacctggacagcctgcca | |
| (Anti-CD3 × | gcatcagctgcaagagcagccagagcctggtgcacaacaacgccaacacctacctgag | |
| Anti- | ctggtatctgcagaagcccggccagagcccccagtccctgatctacaaggtgtccaaca | |
| CD28-L:) | gattcagcggcgtgcccgacagattctccggcagcggctctggcaccgacttcaccctg | |
| | aagatcagccgggtggaagccgaggacgtgggcgtgtactattgtggccagggcaccc | |
| | agtaccccttcacctaggcagcggcaccaaggtggaaatcaagggccagcccaaggc | |
| | cgcccccgacatcgtgctgacacagagccctgctagcctggccgtgtctcctggacaga | |
| | gggccaccatcacctgtagagccagcgagagcgtggaatattacgtgaccagcctgatg | |
| | cagtggtatcagcagaagcccggccagccccccaagctgctgattttcgccgccagcaa | |
| | cgtggaaagcggcgtgccagccagattttccggcagcggctctggcaccgacttcaccc | |
| | tgaccatcaaccccgtggaagccaacgacgtggccaactactactgccagcagagccg | |
| | gaaggtgccctacacctttggccagggcaccaagctggaaatcaagaccaagggcccc | |
| | agccgtacggtggccgctcccagcgtgttcatcttcccacctagcgacgagcagctgaa | |
| | gtccggcacagcctctgtcgtgtgcctgctgaacaacttctacccccgcgaggccaaagt | |
| | gcagtggaaggtggacaacgccctgcagagcggcaacagccaggaaagcgtgaccg | |
| | agcaggacagcaaggactccacctacagcctgagcagcaccctgacactgagcaagg | |
| | ccgactacgagaagcacaaggtgtacgcctgcgaagtgacccaccagggcctgtctag | |
| | ccccgtgaccaagagcttcaaccggggcgagtgt | |

Binding Protein 20 Amino Acid Sequences

| | | |
|---|---|---|
| Heavy | Anti-CD20-H_Knob: | SEQ ID NO: 114 |
| chain A | Qvqlqqpgaelvkpgasvkmsckas [symbol] mhwvkqtpgrglewiga [symbol] | |
| (Anti- | [symbol] synqkfkgkatltadkssstaymqlssltsedsavyyc [symbol] w | |
| CD20- | gagttvtvsaastkgpsvfplapcsrstsestaalgclvkdyfpepvtvswnsgaltsgv | |
| H_knob) | htfpavlqssglyslssvvtvpssslgtktytcnvdhkpsntkvdkrveskygppcppc | |
| | papeflggpsvflfppkpkdtlmisrtpevtcvvvdvsqedpevqfnwyvdgvevh | |
| | naktkpreeqfnstyrvvsyltvlhqdwlngkeykckvsnkglpssiektiskakgqp | |
| | repqvytlppcqeemtknqvslwclvkgfypsdiavewesngqpennykttppvld | |
| | sdgsfflyskltvdksrwqegnvfscsvmhealhnhytqkslslslgk | |
| Light chain | Anti-CD20-L: | SEQ ID NO: 115 |
| A | Qivlsqspailsaspgekvtmtcrasssvsyihwfqqkpgsspkpwiy [symbol] nlasgvp | |
| (Anti- | vrfsgsgsgtsysltisrveaedaatyyc [symbol] ptfgggtkleikrtvaapsvfifpp | |
| CD20-L) | sdeqlksgtasvvcllnnfypreakvqwkvdnalqsgnsqesvteqdskdstyslsstl | |
| | tlskadyekhkvyacevthqglsspvtksfnrgec | |
| Heavy | CD28 × CD3-H_Hole: | SEQ ID NO: 3 |
| chain B | Qvqlvqsgaevvkpgasvkvsckas [symbol] ihwvrqapgqglewigsiy | |
| (CD28 × | [symbol] nyaqkfqgratltvdtsistaymelsrlrsddtavyyc [symbol] | |
| CD3- | [symbol] wgkgttvtvsssqvqlvesgggvvqpgrslrlscaas [symbol] mhwv | |
| H_Hole) | rqapgkqlewvaq [symbol] yatyyadsvkgrftisrddskntlylqmnslra | |
| | edtavyyc [symbol] wgqgtlvtvssrtastkgpsvfplapcsrstsest | |
| | aalgclvkdyfpepvtvswnsgaltsgvhtfpavlqssglyslssvvtvpssslg | |
| | tktytcnvdhkpsntkvdkrveskygppcppcpapeflggpsvflfppkpkd | |
| | tlmisrtpevtcvvvdvsqedpevqfnwyvdgvevhnaktkpreeqfnstyr | |
| | vvsvltvlhqdwlngkeykckvsnkglpssiektiskakgqprepqvctlpps | |
| | qeemtknqvslscavkgfypsdiavewesngqpennykttppvldsdgsffl | |
| | vskltvdksrwqegnvfscsvmhealhnhytqkslslslgk | |
| Light chain | CD3 × CD28-L: | SEQ ID NO: 4 |
| B (CD3 × | Divmtqtplslsvtpgqpasisckss [symbol] lswylqkpgqspqsliy [symbol] n | |
| CD28-L) | rfsgvpdrfsgsgsgtdftlkisrveaedvgvyyc [symbol] ftfgsgtkveikgqpka | |
| | apdiqmtqspsslsasvgdrvtitcqas [symbol] lnwyqqkpgkapklliy [symbol] nlht | |
| | gvpsrfsgsgsgtdftltisslqpediatyyc [symbol] fgqgtkleiktkgpsrtvaap | |
| | svfifppsdeqlksgtasvvcllnnfypreakvqwkvdnalqsgnsqesvteqdskds | |
| | tyslsstltlskadyekhkvyacevthqglsspvtksfnrgec | |

Binding Protein 20 Nucleotide Sequences

| | | |
|---|---|---|
| Heavy | Anti-CD2O-H_Knob: | SEQ ID NO: 116 |
| chain A | caggtgcagctgcagcagcctggcgccgaactcgtgaaacctggcgcctccgtgaaga | |
| (Anti- | tgagctgcaaggccagcggctacaccttcaccagctacaacatgcactgggtcaagcag | |
| CD20- | acccccggcagaggcctggaatggatcggcgccatctaccccggcaacggcgacacc | |
| H_Knob:) | tcctacaaccagaagttcaagggcaaggccaccctgaccgccgacaagagcagcagc | |
| | acagcctacatgcagctgtccagcctgaccagcgaggacagcgccgtgtactactgcgc | |
| | cagaagcacctactacggcggcgactggtacttcaacgtgtggggagccggcaccacc | |
| | gtgacagtgtctgctgcttcgaccaagggcccatcggtgttccctctggccccttgcagca | |
| | gaagcaccagcgaatctacagccgccctgggctgcctcgtgaaggactactttcccgag | |
| | cccgtgaccgtgtcctggaactctggcgctctgacaagcggcgtgcacacctttccagcc | |
| | gtgctccagagcagcggcctgtactctctgagcagcgtcgtgacagtgcccagcagcag | |
| | cctgggcaccaagacctacacctgtaacgtggaccacaagcccagcaacaccaaggtg | |
| | gacaagcgggtggaatctaagtacggccctccctgccctccttgcccagcccctgaattt | |
| | ctgggcggaccctccgtgttcctgttccccccaaagcccaaggacaccctgatgatcagc | |
| | cggacccccgaagtgacctgcgtggtggtggatgtgtcccaggaagatcccgaggtgc | |
| | agttcaattggtacgtggacggcgtggaagtgcacaacgccaagaccaagcccagaga | |
| | ggaacagttcaacagcacctaccgggtggtgtccgtgctgaccgtgctgcaccaggact | |
| | ggctgaacggcaaagagtacaagtgcaaggtgtccaacaagggcctgcccagctccat | |
| | cgagaaaaccatcagcaaggccaagggccagccccgcgagcctcaagtgtataccctg | |
| | cccccttgccaggaagagatgaccaagaaccaggtgtccctgtggtgtctcgtgaaagg | |
| | cttctaccccagcgacattgccgtggaatgggagagcaacggccagcccgagaacaac | |
| | tacaagaccaccccccctgtgctggacagcgacggctcattcttcctgtactccaagctga | |
| | ccgtggacaagagccggtggcaggaaggcaacgtgttcagctgctccgtgatgcacga | |
| | ggccctgcacaaccactacacccagaagtccctgtctctgtccctgggcaag | |
| Light chain | Anti-CD20-L: | SEQ ID NO: 117 |
| A (Anti- | cagatcgtgctgagccagagccctgccatcctgagcgcttccccaggcgagaaagtgac | |
| CD20-L) | catgacctgcagagccagcagcagcgtgtcctacatccactggttccagcagaagcccg | |
| | gcagcagccccaagccttggatctacgccaccagcaatctggccagcggagtgcctgtg | |
| | cggtttagcggctctggcagcggcacaagctacagcctgaccatcagccgggtggaag | |
| | ccgaagatgccgccacctactactgccagcagtggaccagcaacccccccacatttggc | |
| | ggaggcaccaagctggaaatcaagcgtacggtggccgctcccagcgtgttcatcttccc | |
| | acctagcgacgagcagctgaagtccggcacagcctctgtcgtgtgcctgctgaacaactt | |
| | ctacccccgcgaggccaaagtgcagtggaaggtggacaacgccctgcagagcggcaa | |
| | cagccaggaaagcgtgaccgagcaggacagcaaggactccacctacagcctgagcag | |
| | caccctgacactgagcaaggccgactacgagaagcacaaggtgtacgcctgcgaagtg | |
| | acccaccagggcctgtctagccccgtgaccaagagcttcaaccggggcgagtgt | |
| Heavy | CD28 × CD3-H_Hole: | SEQ ID NO: 7 |
| chain B | caggtgcagctggtgcagtctggcgccgaggtcgtgaaacctggcgcctctgtgaaggt | |
| (CD28 × | gtcctgcaaggccagcggctacacctttaccagctactacatccactgggtgcgccaggc | |
| CD3- | ccctggacagggactggaatggatcggcagcatctaccccggcaacgtgaacaccaac | |
| H_Hole:) | tacgcccagaagttccagggcagagccaccctgaccgtggacaccagcatcagcaccg | |
| | cctacatggaactgagccggctgagaagcgacgacaccgccgtgtactactgcacccg | |
| | gtcccactacggcctggattggaacttcgacgtgtggggcaagggcaccaccgtgacag | |
| | tgtctagcagccaggtgcagctggtggaatctggcggcggagtggtgcagcctggcag | |
| | aagcctgagactgagctgtgccgccagcggcttcaccttcaccaaggcctggatgcact | |
| | gggtgcgccaggcccctggaaagcagctggaatgggtggcccagatcaaggacaaga | |
| | gcaacagctacgccacctactacgccgacagcgtgaagggccggttcaccatcagccg | |
| | ggacgacagcaagaacaccctgtacctgcagatgaacagcctgcgggccgaggacac | |
| | cgccgtgtactactgtcggggcgtgtactatgccctgagccccttcgattactggggccag | |
| | ggaaccctcgtgaccgtgtctagtcggaccgccagcacaaagggcccatcggtgttccc | |
| | tctggccccttgcagcagaagcaccagcgaatctacagccgccctgggctgcctcgtga | |
| | aggactactttcccgagcccgtgaccgtgtcctggaactctggcgctctgacaagcggcg | |
| | tgcacacctttccagccgtgctccagagcagcggcctgtactctctgagcagcgtcgtga | |
| | cagtgcccagcagcagcctgggcaccaagacctacacctgtaacgtggaccacaagcc | |
| | cagcaacaccaaggtggacaagcgggtggaatctaagtacggccctccctgccctcctt | |
| | gcccagcccctgaatttctgggcggaccctccgtgttcctgttccccccaaagcccaagg | |
| | acaccctgatgatcagccggacccccgaagtgacctgcgtggtggtggatgtgtcccag | |
| | gaagatcccgaggtgcagttcaattggtacgtggacggcgtggaagtgcacaacgccaa | |
| | gaccaagcccagagaggaacagttcaacagcacctaccgggtggtgtccgtgctgacc | |
| | gtgctgcaccaggactggctgaacggcaaagagtacaagtgcaaggtgtccaacaagg | |
| | gcctgcccagctccatcgagaaaaccatcagcaaggccaagggccagccccgcgagc | |
| | ctcaagtgtgtaccctgccccctagccaggaagagatgaccaagaaccaggtgtccctg | |
| | agctgtgccgtgaaaggcttctaccccagcgacattgccgtggaatgggagagcaacgg | |
| | ccagcccgagaacaactacaagaccaccccccctgtgctggacagcgacggctcattct | |
| | tcctggtgtccaagctgaccgtggacaagagccggtggcaggaaggcaacgtgttcag | |
| | ctgctccgtgatgcacgaggccctgcacaaccactacacccagaagtccctgtctctgtc | |
| | cctgggcaag | |
| Light chain | CD3 × CD28-L_Hole: | SEQ ID NO: 8 |
| B | gacatcgtgatgacccagacccccctgagcctgagcgtgacacctggacagcctgcca | |
| (CD3 × | gcatcagctgcaagagcagccagagcctggtgcacaacaacgccaacacctacctgag | |
| CD28- | ctggtatctgcagaagcccggccagagcccccagtccctgatctacaaggtgtccaaca | |
| L_Hole) | gattcagcggcgtgcccgacagattctccggcagcggctctggcaccgacttcaccctg | |
| | aagatcagccgggtggaagccgaggacgtgggcgtgtactattgtggccagggcaccc | |
| | agtaccccttcacctttggcagcggcaccaaggtggaaatcaagggccagcccaaggc | |
| | cgcccccgacatccagatgacccagagccccagcagcctgtctgccagcgtgggcgac | |
| | agagtgaccatcacctgtcaggccagccagaacatctacgtgtggctgaactggtatcag | |
| | cagaagcccggcaaggcccccaagctgctgatctacaaggccagcaacctgcacacc | |
| | ggcgtgcccagcagattttctggcagcggctccggcaccgacttcaccctgacaatcag | |
| | ctccctgcagcccgaggacattgccacctactactgccagcagggccagacctacccct | |
| | acacctttggccagggcaccaagctggaaatcaagaccaagggccccagccgtacggt | |
| | ggccgctcccagcgtgttcatcttcccacctagcgacgagcagctgaagtccggcacag | |
| | cctctgtcgtgtgcctgctgaacaacttctacccccgcgaggccaaagtgcagtggaagg | |
| | tggacaacgccctgcagagcggcaacagccaggaaagcgtgaccgagcaggacagc | |
| | aaggactccacctacagcctgagcagcaccctgacactgagcaaggccgactacgaga | |
| | agcacaaggtgtacgcctgcgaagtgacccaccagggcctgtctagccccgtgaccaa | |
| | gagcttcaaccggggcgagtgt | |

Binding Protein 21 Amino Acid Sequences

| | | |
|---|---|---|
| Heavy | Anti-CD20-H_Knob: | SEQ ID NO: 114 |
| chain | qvqlqqpgaelvkpgasykmsckas [symbol] mhwvkqtpgrglewiga [symbol] synqkfk | |
| A | gkatltadkssstaymqlssltsedsavyyc [symbol] wgagttvtvsaastkgpsvfplap | |
| (Anti- | csrstsestaalgclvkdyfpepvtvswnsgaltsgvhtfpavlqssglyslssvvtvpssslgtktytcnv | |
| CD20- | dhkpsntkvdkrveskygppcppcpapeflggpsvflfppkpkdtlmisrtpevtcvvvdvsqedp | |
| H_knob) | evqfnwyvdgvevhnaktkpreeqfnstyrvvsvltvlhqdwlngkeykckvsnkglpssiektisk | |
| | akgqprepqvyttppcqeemtknqvslwchkgfypsdiavewesngqpennykttppvldsdgs | |
| | fflyskltvdksrwqegnyfscsvmhealhnhytqkslslslgk | |
| Light | Anti-CD20-L: | SEQ ID NO: 115 |
| chain | qivlsqspailsaspgekytmtcras ssvsy ihwfqqkpgsspkpwiy [symbol] nlasgvpvrfsgsgsgts | |
| A | ysltisrveaedaatyyc [symbol] ptfgggtkleikrtvaapsyfifppsdeqlksgtasvvcllnnfyp | |
| (Anti- | reakvqwkvdnalqsgnsqesvteqdskdstyslsstltlskadyekhkvyacevthqglsspvtksfn | |
| CD20-L) | rgec | |
| Heavy | CD28 × CD3-H_Hole: | SEQ ID NO: 9 |
| chain B | qvqlqesgpglvkpsqtlsltctvsgfslsdygvhwvrqppgkglewlgviwagggtnynp | |
| (CD28 × | slksrktiskdtsknqvslklssvtaadtavyycardkgysyyysmdywgqgttvtvsssqv | |
| CD3- | qlvesgggvvqpgrslrlscaasgftftkawmhwvrqapgkqlewvaqikdksnsyatyy | |
| H_Hole) | adsvkgrftisrddskntlylqmnslraedtavyycrgvyyalspfdywgqgtlvtvssrtast | |
| | kgpsvfplapcsrstsestaalgclvkdyfpepvtvswnsgaltsgvhtfpavlqssglyslssv | |
| | vtvpssslgtktytcnvdhkpsntkvdkrveskygppcppcpapeflggpsvflfppkpkdt | |
| | lmisrtpevtcvvvdvsqedpevqfnwyvdgvevhnaktkpreeqfnstyrvvsvltvlhq | |
| | dwlngkeykckvsnkglpssiektiskakgqprepqvctlppsqeemtknqvslscavkgf | |
| | ypsdiavewesngqpennykttppvldsdgsfflvskltvdksrwqegnvfscsmhealh | |
| | nhytqkslslslgk | |
| Light | CD3 × CD28-L | SEQ ID NO: 10 |
| chain B | divmtqtplslsvtpgqpasisckssqslyhnnantylswylqkpgqspqsliykvsnrfsgvpdrfsg | |
| (CD3 × | sgsgtdftlkisrveaedvgvyycgqgtqypftfgsgtkveikgqpkaapdivltqspaslavspgqrat | |
| CD28- | itcrasesveyyvtslmqwyqqkpgqppkilifaasnvesgyparfsgsgsgtdftltinpveandvan | |
| L) | yycqqsrkvpytfgqgtkleiktkgpsrtvaapsvfifppsdeqlksgtasvvcllnnfypreakvqwk | |
| | vdnalqsgnsqesvteqdskdstyslssddskadyekhkvyacevthqglsspvtksfnrgec | |

Binding Protein 21 Nucleotide Sequences

| | | |
|---|---|---|
| Heavy | Anti-CD20-H_Knob: | SEQ ID NO: 116 |
| chain A | caggtgcagctgcagcagcctggcgccgaactcgtgaaacctggcgcctccgtgaagatgagctgca | |
| (Anti- | aggccagcggctacaccttcaccagctacaacatgcactgggtcaagcagacccccggcagaggcct | |
| CD20- | ggaatggatcggcgccatctaccccggcaacggcgacacctcctacaaccagaagttcaagggcaag | |
| H_Knob:) | gccaccctgaccgccgacaagagcagcagcacagcctacatgcagctgtccagcctgaccagcgag | |
| | gacagcgccgtgtactactgcgccagaagcacctactacggcggcgactggtacttcaacgtgtgggg | |
| | agccggcaccaccgtgacagtgtctgctgcttcgaccaagggcccatcggtgttccctctggccccttgc | |
| | agcagaagcaccagcgaatctacagccgccctgggctgcctcgtgaaggactactacccgagcccgt | |
| | gaccgtgtcctggaactctggcgctctgacaagcggcgtgcacacctaccagccgtgctccagagcag | |
| | cggcctgtactctctgagcagcgtcgtgacagtgcccagcagcagcctgggcaccaagacctacacct | |
| | gtaacgtggaccacaagcccagcaacaccaaggtggacaagcgggtggaatctaagtacggccctcc | |
| | ctgccctccttgcccagcccctgaatttctgggcggaccctccgtgacctgaccccccaaagcccaag | |
| | gacaccctgatgatcagccggacccccgaagtgacctgcgtggtggtggatgtgtcccaggaagatcc | |
| | cgaggtgcagttcaattggtacgtggacggcgtggaagtgcacaacgccaagaccaagcccagagag | |
| | gaacagttcaacagcacctaccgggtggtgtccgtgctgaccgtgctgcaccaggactggctgaacgg | |
| | caaagagtacaagtgcaaggtgtccaacaagggcctgcccagctccatcgagaaaaccatcagcaag | |
| | gccaagggccagccccgcgagcctcaagtgtataccctgcccccttgccaggaagagatgaccaaga | |
| | accaggtgtccctgtggtgtctcgtgaaaggcttctaccccagcgacattgccgtggaatgggagagca | |
| | acggccagcccgagaacaactacaagaccaccccccctgtgctggacagcgacggctcattcacctgt | |
| | actccaagctgaccgtggacaagagccggtggcaggaaggcaacgtgttcagctgctccgtgatgcac | |
| | gaggccctgcacaaccactacacccagaagtccctgtctctgtccctgggcaag | |
| Light | Anti-CD20-L: | SEQ ID NO: 117 |
| chain A | cagatcgtgctgagccagagccctgccatcctgagcgcttccccaggcgagaaagtgaccatgacctg | |
| (Anti- | cagagccagcagcagcgtgtcctacatccactggttccagcagaagcccggcagcagccccaagcctt | |
| CD20- | ggatctacgccaccagcaatctggccagcggagtgcctgtgcggtttagcggctctggcagcggcaca | |
| L:) | agctacagcctgaccatcagccgggtggaagccgaagatgccgccacctactactgccagcagtgga | |
| | ccagcaacccccccacatttggcggaggcaccaagctggaaatcaagcgtacggtggccgctcccag | |
| | cgtgttcatcttcccacctagcgacgagcagctgaagtccggcacagcctctgtcgtgtgcctgctgaac | |
| | aacttctacccccgcgaggccaaagtgcagtggaaggtggacaacgccctgcagagcggcaacagc | |
| | caggaaagcgtgaccgagcaggacagcaaggactccacctacagcctgagcagcaccctgacactg | |
| | agcaaggccgactacgagaagcacaaggtgtacgcctgcgaagtgacccaccagggcctgtctagcc | |
| | ccgtgaccaagagcttcaaccggggcgagtgt | |
| Heavy | CD28 × CD3-H_Hole: | SEQ ID NO: 118 |
| chain B | caggtgcagctgcaggaatctggccctggcctcgtgaagcctagccagaccctgagcctgacctgtac | |
| (CD28 × | cgtgtccggcttcagcctgagcgactacggcgtgcactgggtgcgccagccacctggaaaaggcctg | |
| CD3- | gaatggctgggcgtgatctgggctggcggaggcaccaactacaaccccagcctgaagtccagaaaga | |
| H_Hole: | ccatcagcaaggacaccagcaagaaccaggtgtccctgaagctgagcagcgtgacagccgccgatac | |
| | cgccgtgtactactgcgccagagacaagggctacagctactactacagcatggactactggggccagg | |
| | gcaccaccgtgaccgtgtcatcctctcaggtgcagctggtggaatctggcggcggagtggtgcagcct | |
| | ggcagaagcctgagactgagctgtgccgccagcggcttcaccttcaccaaggcctggatgcactgggt | |
| | gcgccaggcccctggaaagcagctggaatgggtggcccagatcaaggacaagagcaacagctacgc | |
| | cacctactacgccgacagcgtgaagggccggttcaccatcagccgggacgacagcaagaacaccctg | |
| | tacctgcagatgaacagcctgcgggccgaggacaccgccgtgtactactgtcggggcgtgtactatgc | |
| | cctgagccccttcgattactggggccagggaaccctcgtgaccgtgtctagtcggaccgcttcgaccaa | |
| | gggcccatcggtgttccctctggccccttgcagcagaagcaccagcgaatctacagccgccctgggct | |
| | gcctcgtgaaggactactttcccgagcccgtgaccgtgtcctggaactctggcgctctgacaagcggcg | |
| | tgcacacctttccagccgtgctccagagcagcggcctgtactctctgagcagcgtcgtgacagtgccca | |
| | gcagcagcctgggcaccaagacctacacctgtaacgtggaccacaagcccagcaacaccaaggtgg | |
| | acaagcgggtggaatctaagtacggccctccctgccctccttgcccagcccctgaatttctgggcggac | |
| | cctccgtgttcctgttccccccaaagcccaaggacaccctgatgatcagccggacccccgaagtgacct | |
| | gcgtggtggtggatgtgtcccaggaagatcccgaggtgcagttcaattggtacgtggacggcgtggaa | |
| | gtgcacaacgccaagaccaagcccagagaggaacagttcaacagcacctaccgggtggtgtccgtgc | |
| | tgaccgtgctgcaccaggactggctgaacggcaaagagtacaagtgcaaggtgtccaacaagggcct | |
| | gcccagctccatcgagaaaaccatcagcaaggccaagggccagccccgcgagcctcaagtgtgtacc | |
| | ctgccccctagccaggaagagatgaccaagaaccaggtgtccctgagctgtgccgtgaaaggcttcta | |
| | ccccagcgacattgccgtggaatgggagagcaacggccagcccgagaacaactacaagaccacccc | |
| | ccctgtgctggacagcgacggctcattcttcctggtgtccaagctgaccgtggacaagagccggtggca | |
| | ggaaggcaacgtgttcagctgctccgtgatgcacgaggccctgcacaaccactacacccagaagtccc | |
| | tgtctctgtccctgggcaag | |
| Light | (CD3_CD28-L:: | SEQ ID NO: 119 |
| chain B | gacatcgtgatgacccagacccccctgagcctgagcgtgacacctggacagcctgccagcatcagctg | |
| (CD3_CD28- | caagagcagccagagcctggtgcacaacaacgccaacacctacctgagctggtatctgcagaagccc | |
| L:) | ggccagagcccccagtccctgatctacaaggtgtccaacagattcagcggcgtgcccgacagattctcc | |
| | ggcagcggctctggcaccgacttcaccctgaagatcagccgggtggaagccgaggacgtgggcgtgt | |
| | actattgtggccagggcacccagtaccccttcacctttggcagcggcaccaaggtggaaatcaagggc | |
| | cagcccaaggccgcccccgacatcgtgctgacacagagccctgctagcctggccgtgtctcctggaca | |
| | gagggccaccatcacctgtagagccagcgagagcgtggaatattacgtgaccagcctgatgcagtggt | |
| | atcagcagaagcccggccagccccccaagctgctgattttcgccgccagcaacgtggaaagcggcgt | |
| | gccagccagattttccggcagcggctctggcaccgacttcaccctgaccatcaaccccgtggaagcca | |
| | acgacgtggccaactactactgccagcagagccggaaggtgccctacacctttggccagggcaccaa | |
| | gctggaaatcaagaccaagggccccagccgtacggtggccgctcccagcgtgttcatcttcccacctag | |
| | cgacgagcagctgaagtccggcacagcctctgtcgtgtgcctgctgaacaacttctacccccgcgagg | |
| | ccaaagtgcagtggaaggtggacaacgccctgcagagcggcaacagccaggaaagcgtgaccgag | |
| | caggacagcaaggactccacctacagcctgagcagcaccctgacactgagcaaggccgactacgag | |
| | aagcacaaggtgtacgcctgcgaagtgacccaccagggcctgtctagccccgtgaccaagagcttcaa | |
| | ccggggcgagtgt | |

TABLE 3
Heavy and light chain sequences of binding proteins
specifically directed to IL-4, IL-13 and/or TNFa.
Binding Protein 9 Amino Acid Sequences

| | | |
|---|---|---|
| Heavy | HC: | SEQ ID NO: 60 |
| chain A | EVQLVESGGGLVQPGRSLRLSCAASGFTFDDYAMHWVRQAPG | |
| | KGLEWVSAITWNSGHIDYADSVEGRFTISRDNAKNSLYLQMN | |
| | SLRAEDTAVYYCAKVSYLSTASSLDYWGQGTLVTVSSASTKG | |
| | PSVFPLAPSSKSTSGGTAALGCLVKDYFPEPVTVSWNSGALT | |
| | SGVHTFPAVLQSSGLYSLSSVVTVPSSSLGTQTYICNVNHKP | |
| | SNTKVDKKVEPKSCDKTHTCPPCPAPELLGGPSVFLFPPKPK | |
| | DTLMISRTPEVTCVVVDVSHEDPEVKFNWYVDGVEVHNAKTK | |
| | PREEQYNSTYRVVSVLTVLHQDWLNGKEYKCKVSNKALPAPI | |
| | EKTISKAKGQPREPQVCTLPPSRDELTKNQVSLSCAVKGFYP | |
| | SDIAVEWESNGQPENNYKTTPPVLDSDGSFFLVSKLTVDKSR | |
| | WQQGNVFSCSVMHEALHNHYTQKSLSLSPG | |
| Light | LC: | SEQ ID NO: 61 |
| chain A | DIQMTQSPSSLSASVGDRVTITCRASQGIRNYLAWYQQKPGK | |
| | APKLLIYAASTLQSGVPSRFSGSGSGTDFTLTISSLQPEDVA | |
| | TYYCQRYNRAPYTFGQGTKVEIKGQPKAAPSVTLFPPSSEEL | |
| | QANKATLVCLISDFYPGAVTVAWKADSSPVKAGVETTTPSKQ | |
| | SNNKYAASSYLSLTPEQWKSHRSYSCQVTHEGSTVEKTVAPT | |
| | ECS | |
| Heavy | HC: | SEQ ID NO: 62 |
| chain B | QVQLQQSGPELVKPGASVKISCKASGYSFTSYWIHWIKQRPG | |
| | QGLEWIGMIDPSDGETRLNQRFQGRATLTVDESTSTAYMQLR | |
| | SPTSEDSAVYYCTRLKEYGNYDSFYFDVWGAGTLVTVSSEVQ | |
| | LKESGPGLVAPGGSLSITCTVSGFSLTDSSINWVRQPPGKGL | |
| | EWLGMIWGDGRIDYADALKSRLSISKDSSKSQVFLEMTSLRT | |
| | DDTATYYCARDGYFPYAMDFWGQGTSVTVSSASTKGPSVFPL | |
| | APSSKSTSGGTAALGCLVKDYFPEPVTVSWNSGALTSGVHTF | |
| | PAVLQSSGLYSLSSVVTVPSSSLGTQTYICNVNHKPSNTKVD | |
| | KKVEPKSCDKTHTCPPCPAPELLGGPSVFLFPPKPKDTLMIS | |
| | RTPEVTCVVVDVSHEDPEVKFNWYVDGVEVHNAKTKPREEQY | |
| | NSTYRVVSVLTVLHQDWLNGKEYKCKVSNKALPAPIEKTISK | |
| | AKGQPREPQVYTLPPCRDELTKNQVSLWCLVKGFYPSDIAVE | |
| | WESNGQPENNYKTTPPVLDSDGSFFLYSKLTVDKSRWQQGNV | |
| | FSCSVMHEALHNRFTQKSLSLSPG | |
| Light | LC: | SEQ ID NO: 63 |
| chain B | DIVLTQSPASLAVSLGQRATISCRASESVDSYGQSYMHWYQQ | |
| | KAGQPPKLLIYLASNLESGVPARFSGSGSRTDFTLTIDPVQA | |
| | EDAATYYCQQNAEDSRTFGGGTKLEIKGGSGSSGSGGDIQMT | |
| | QSPASLSVSVGDTITLTCHASQNIDVWLSWFQQKPGNIPKLL | |
| | IYKASNLHTGVPSRFSGSGSGTGFTLTISSLQPEDIATYYC | |
| | QQAHSYPFTFGGGTKLEIKGGSGSSGSGGRTVAAPSVFIFPP | |
| | SDEQLKSGTASVVCLLNNFYPREAKVQWKVDNALQSGNSQES | |
| | VTEQDSKDSTYSLSSTLTLSKADYEKHKVYACEVTHQGLSSP | |
| | VTKSFNRGEC | |

Binding Protein 9 Nucleotide Sequences

| | | |
|---|---|---|
| Heavy | HC: | SEQ ID NO: 64 |
| chain A | gaggtgcagctggtggaaagcggcggaggactggtgcagccc | |
| | ggcagaagcctgagactgagctgcgccgccagcggcttcacc | |
| | ttcgacgactacgccatgcactgggtccgccaggcccctggc | |
| | aagggcctggaatgggtgtccgccatcacctggaacagcggc | |
| | cacatcgactacgccgacagcgtggaaggccggttcaccatc | |
| | agccgggacaacgccaagaacagcctgtacctgcagatgaac | |
| | agcctgcgggccgaggacaccgccgtgtactactgcgccaag | |
| | gtgtcctacctgagcaccgccagcagcctggactactggggc | |
| | cagggcaccctggtgacagtgtccagcgcttccaccaagggc | |
| | cccagcgtgttccccctggcccctagcagcaagagcacatct | |
| | ggcggcacagccgccctgggctgcctggtcaaggactacttc | |
| | cccgagcccgtgacagtgtcctggaactctggcgccctgacc | |
| | agcggagtgcataccttccctgccgtgctgcagtccagcggc | |
| | ctgtacagcctgagcagcgtggtcacagtgcccagcagcagc | |
| | ctgggcacccagacctacatctgcaacgtgaaccacaagccc | |
| | agcaacaccaaggtggacaagaaggtggaacccaagagctgc | |
| | gacaagacccacacctgtcccccctgccctgcccctgaactg | |
| | ctgggcggaccctccgtgttcctgttccccccaaagcccaag | |
| | gacaccctgatgatcagccggacccccgaagtgacctgcgtg | |
| | gtggtggacgtgtcccacgaggaccctgaagtgaagttcaat | |
| | tggtacgtggacggcgtggaagtgcataacgccaagaccaag | |
| | cccagagaggaacagtacaacagcacctaccgggtggtgtcc | |
| | gtgctgaccgtgctgcaccaggactggctgaacggcaaagag | |
| | tacaagtgcaaggtgtccaacaaggccctgcctgcccccatc | |
| | gagaaaaccatcagcaaggccaagggccagcctagagagccc | |
| | caagtctgcaccctgccccccagcagagatgagctgaccaag | |
| | aaccaggtgtccctgagctgcgccgtgaagggcttctacccc | |
| | agcgatatcgccgtggaatgggagagcaacggccagcccgag | |
| | aacaactacaagaccaccccccctgtgctggacagcgacggc | |
| | tcattcttcctggtgtccaagctgacagtggacaagagccgg | |
| | tggcagcagggcaacgtgttcagctgcagcgtgatgcacgag | |
| | gccctgcacaaccattacacccagaagtccctgagcctgagc | |
| | cccggc | |
| Light | LC: | SEQ ID NO: 65 |
| chain A | gacatccagatgacccagagccccagcagcctgagcgccagc | |
| | gtgggcgacagagtgaccatcacctgtcgggccagccagggc | |
| | atccggaactacctggcctggtatcagcagaagcccggcaag | |
| | gcccccaagctgctgatctacgccgccagcacactgcagagc | |
| | ggcgtgcccagcagattcagcggcagcggctccggcaccgac | |
| | ttcaccctgaccatcagcagcctgcagcccgaggacgtggcc | |
| | acctactactgccagcggtacaacagagccccctacaccttc | |
| | ggccagggcaccaaggtggaaatcaagggacagcccaaggct | |
| | gccccctcggtcaccctgttccccccaagcagcgaggaactg | |
| | caggccaacaaggccaccctcgtgtgcctgatcagcgacttc | |
| | taccctggcgccgtgaccgtggcctggaaggccgatagctct | |
| | cccgtgaaggccggcgtggaaaccaccacccccagcaagcag | |
| | agcaacaacaaatacgccgcctccagctacctgagcctgacc | |
| | cccgagcagtggaagtcccaccggtcctacagctgccaggtc | |
| | acacacgagggcagcaccgtggaaaagaccgtggcccccacc | |
| | gagtgcagc | |
| Heavy | HC: | SEQ ID NO: 66 |
| chain B | caggtgcagctgcagcagagcggccctgagctggtcaagcct | |
| | ggcgccagcgtgaagatcagctgcaaggccagcggctacagc | |
| | ttcaccagctactggatccactggatcaagcagcggcctggc | |
| | cagggcctggaatggatcggcatgatcgaccccagcgacggc | |
| | gagacacggctgaaccagagattccagggcagagccaccctg | |
| | accgtggacgagagcaccagcaccgcctacatgcagctgcgg | |
| | agccccaccagcgaggacagcgccgtgtactactgcacccgg | |
| | ctgaaagagtacggcaactacgacagatctacttcgacgtgt | |
| | ggggagccggcaccctggtcaccgtgtccagcgaagtgcagc | |
| | tgaaagaaagcggccctggcctggtggcccctggcggcagcc | |
| | tgagcatcacctgtaccgtgtccggcttcagcctgaccgaca | |
| | gcagcatcaactgggtccgacagccccctggcaagggcctcg | |
| | agtggctgggaatgatctggggcgacggccggatcgactacg | |
| | ccgacgccctgaagtcccggctgagcatcagcaaggacagca | |
| | gcaagagccaggtgttcctggaaatgaccagcctgcggaccg | |
| | acgacaccgccacctactactgcgccagggacggctacttcc | |
| | cctacgccatggatnctggggccagggcaccagcgtgaccgt | |
| | gtcctctgcttccaccaagggccccagcgtgttccctctggc | |
| | ccctagcagcaagagcacatctggcggaacagccgccctggg | |
| | ctgcctggtcaaggactactttcccgagcccgtgaccgtgtc | |
| | ctggaactctggtgccctgacaagcggagtgcataccttccc | |
| | tgccgtgctgcagagcagcggcctgtactctctgagcagcgt | |
| | ggtcaccgtgccaagcagcagcctgggcacccagacctacat | |
| | ctgcaacgtgaaccacaagccctccaacaccaaggtggacaa | |
| | gaaggtggaacccaagagctgcgacaagacccacacctgtcc | |
| | tccctgtcctgcccctgaactgctgggcggaccctccgtgtt | |
| | cctgttccctccaaagcccaaggataccctgatgatcagccg | |
| | gacccctgaagtgacctgcgtggtggtggacgtgtcccacga | |
| | ggatcccgaagtgaagttcaattggtacgtggacggcgtgga | |
| | agtgcataacgccaagaccaagcccagagaggaacagtacaa | |
| | cagcacctaccgggtggtgtccgtgctgacagtgctgcacca | |
| | ggactggctgaacggcaaagagtacaagtgcaaggtgtccaa | |
| | caaggccctgccagcccctatcgagaaaaccatcagcaaggc | |
| | caagggccagccccgcgagcctcaggtgtacacactgcctcc | |
| | atgccgggacgagctgaccaagaaccaggtgtccctgtggtg | |
| | cctcgtgaagggcttctacccctccgatatcgccgtggaatg | |
| | ggagagcaacggccagcccgagaacaactacaagaccacccc | |
| | tcccgtgctggacagcgacggctcattcttcctgtacagcaa | |
| | gctgaccgtggacaagtcccggtggcagcagggcaacgtgtt | |
| | cagctgctctgtgatgcacgaggccctgcacaaccggttcac | |
| | ccagaagtccctgagcctgagccctggc | |
| Light | LC: | SEQ ID NO: 67 |
| chain B | Gacatcgtgctgacccagagccctgccagcctggccgtgtct | |
| | ctgggccagagagccaccatcagctgccgggccagcgagagc | |
| | gtggacagctacggccagagctacatgcactggtatcagcag | |
| | aaggccggccagccccccaagctgctgatctacctggccagc | |
| | aacctggaaagcggcgtgcccgccagattcagcggcagcggc | |
| | agcagaaccgacttcaccctgaccatcgaccccgtgcaggcc | |
| | gaggacgccgccacctactactgccagcagaacgccgaggac | |
| | agccggaccttcggcggaggcaccaagctggaaatcaagggc | |
| | ggctccggcagcagcggctctggcggcgatatccagatgacc | |
| | cagtcccccgcctccctgagcgtgtccgtgggcgacaccatc | |
| | accctgacatgccacgccagccagaacatcgacgtgtggctg | |
| | agctggttccagcagaagcctggcaacatccctaagctgctc | |
| | atctataaggcctccaacctgcacaccggcgtgcccagcagg | |
| | ttttccggctctggcagcggcaccggctttaccctgacaatc | |
| | agcagcctgcagcccgaggatatcgccacatattactgtcag | |
| | caggcccacagctaccccttcacctttggcggcggaacaaag | |
| | ctcgagattaagggcggcagcggaagctccggctccggcgga | |
| | cgtacggtggccgctccttccgtgttcatcttccctccctcc | |
| | gacgagcagctgaagtccggcaccgcctccgtggtgtgtctg | |
| | ctgaacaacttctaccctcgggaggccaaggtgcagtggaag | |
| | gtggacaacgccctgcagtccggcaactcccaggagtccgtc | |
| | accgagcaggactccaaggacagcacctactccctgtcctcc | |
| | accctgaccctgtccaaggccgactacgagaagcacaaggtg | |
| | tacgcctgtgaggtgacccaccagggcctgtccagccctgtg | |
| | accaagtccttcaaccggggcgagtgc | |

Binding Protein 10 Amino Acid Sequences

| | | |
|---|---|---|
| Heavy | HC: | SEQ ID NO: 60 |
| chain A | EVQLVESGGGLVQPGRSLRLSCAASGFTFDDYAMHWVRQAPG | |
| | KGLEWVSAITWNSGHIDYADSVEGRFTISRDNAKNSLYLQMN | |
| | SLRAEDTAVYYCAKVSYLSTASSLDYWGQGTLVTVSSASTKG | |
| | PSVFPLAPSSKSTSGGTAALGCLVKDYFPEPVTVSWNSGALT | |
| | SGVHTFPAVLQSSGLYSLSSVVTVPSSSLGTQTYICNVNHKP | |
| | SNTKVDKKVEPKSCDKTHTCPPCPAPELLGGPSVFLFPPKPK | |
| | DTLMISRTPEVTCVVVDVSHEDPEVKFNWYVDGVEVHNAKTK | |
| | PREEQYNSTYRVVSVLTVLHQDWLNGKEYKCKVSNKALPAPI | |
| | EKTISKAKGQPREPQVCTLPPSRDELTKNQVSLSCAVKGFYP | |
| | SDIAVEWESNGQPENNYKTTPPVLDSDGSFFLVSKLTVDKSR | |
| | WQQGNVFSCSVMHEALHNHYTQKSLSLSPG | |
| Light | LC: | SEQ ID NO: 61 |
| chain A | DIQMTQSPSSLSASVGDRVTITCRASQGIRNYLAWYQQKPGK | |
| | APKLLIYAASTLQSGVPSRFSGSGSGTDFTLTISSLQPEDVA | |
| | TYYCQRYNRAPYTFGQGTKVEIKGQPKAAPSVTLFPPSSEEL | |
| | QANKATLVCLISDFYPGAVTVAWKADSSPVKAGVETTTPSKQ | |
| | SNNKYAASSYLSLTPEQWKSHRSYSCQVTHEGSTVEKTVAPT | |
| | ECS | |
| Heavy | HC: | SEQ ID NO: 68 |
| chain B | EVQLKESGPGLVAPGGSLSITCTVSGFSLTDSSINWVRQPPG | |
| | KGLEWLGMIWGDGRIDYADALKSRLSISKDSSKSQVFLEMTS | |
| | LRTDDTATYYCARDGYFPYAMDFWGQGTSVTVSSQVQLQQSG | |
| | PELVKPGASVKISCKASGYSFTSYWIHWIKQRPGQGLEWIG | |
| | MIDPSDGETRLNQRFQGRATLTVDESTSTAYMQLRSPTSEDS | |
| | AVYYCTRLKEYGNYDSFYFDVWGAGTLVTVSSASTKGPSVFP | |
| | LAPSSKSTSGGTAALGCLVKDYFPEPVTVSWNSGALTSGVHT | |
| | FPAVLQSSGLYSLSSVVTVPSSSLGTQTYICNVNHKPSNTKV | |
| | DKKVEPKSCDKTHTCPPCPAPELLGGPSVFLFPPKPKDTLMI | |
| | SRTPEVTCVVVDVSHEDPEVKFNWYVDGVEVHNAKTKPREEQ | |
| | YNSTYRVVSVLTVLHQDWLNGKEYKCKVSNKALPAPIEKTIS | |
| | KAKGQPREPQVYTLPPCRDELTKNQVSLWCLVKGFYPSDIAV | |
| | EWESNGQPENNYKTTPPVLDSDGSFFLYSKLTVDKSRWQQGN | |
| | VFSCSVMHEALHNRFTQKSLSLSPG | |
| Light | LC: | SEQ ID NO: 69 |
| chain B | DIQMTQSPASLSVSVGDTITLTCHASQNIDVWLSWFQQKPGN | |
| | IPKLLIYKASNLHTGVPSRFSGSGSGTGFTLTISSLQPEDIA | |
| | TYYCQQAHSYPFTFGGGTKLEIKGGSGSSGSGGDIVLTQSPA | |
| | SLAVSLGQRATISCRASESVDSYGQSYMHWYQQKAGQPPKLLIY | |
| | LASNLESGVPARFSGSGSRTDFTLTIDPVQAEDAATYYC | |
| | QQNAEDSRTFGGGTKLEIKGGSGSSGSGGRTVAAPSVFIFPP | |
| | SDEQLKSGTASVVCLLNNFYPREAKVQWKVDNALQSGNSQES | |
| | VTEQDSKDSTYSLSSTLTLSKADYEKHKVYACEVTHQGLSSP | |
| | VTKSFNRGEC | |

Binding Protein_10 Nucleotide Sequences

| | | |
|---|---|---|
| Heavy | HC: | SEQ ID NO: 64 |
| chain A | gaggtgcagctggtggaaagcggcggaggactggtgcagccc | |
| | ggcagaagcctgagactgagctgcgccgccagcggcttcacc | |
| | ttcgacgactacgccatgcactgggtccgccaggcccctggc | |
| | aagggcctggaatgggtgtccgccatcacctggaacagcggc | |
| | cacatcgactacgccgacagcgtggaaggccggttcaccatc | |
| | agccgggacaacgccaagaacagcctgtacctgcagatgaac | |
| | agcctgcgggccgaggacaccgccgtgtactactgcgccaag | |
| | gtgtcctacctgagcaccgccagcagcctggactactggggc | |
| | cagggcaccctggtgacagtgtccagcgcttccaccaagggc | |
| | cccagcgtgttccccctggcccctagcagcaagagcacatct | |
| | ggcggcacagccgccctgggctgcctggtcaaggactacttc | |
| | cccgagcccgtgacagtgtcctggaactctggcgccctgacc | |
| | agcggagtgcataccttccctgccgtgctgcagtccagcggc | |
| | ctgtacagcctgagcagcgtggtcacagtgcccagcagcagc | |
| | ctgggcacccagacctacatctgcaacgtgaaccacaagccc | |
| | agcaacaccaaggtggacaagaaggtggaacccaagagctgc | |
| | gacaagacccacacctgtcccccctgccctgcccctgaactg | |
| | ctgggcggaccctccgtgttcctgttccccccaaagcccaag | |
| | gacaccctgatgatcagccggacccccgaagtgacctgcgtg | |
| | gtggtggacgtgtcccacgaggaccctgaagtgaagttcaat | |
| | tggtacgtggacggcgtggaagtgcataacgccaagaccaag | |
| | cccagagaggaacagtacaacagcacctaccgggtggtgtcc | |
| | gtgctgaccgtgctgcaccaggactggctgaacggcaaagag | |
| | tacaagtgcaaggtgtccaacaaggccctgcctgcccccatc | |
| | gagaaaaccatcagcaaggccaagggccagcctagagagccc | |
| | caagtctgcaccctgccccccagcagagatgagctgaccaag | |
| | aaccaggtgtccctgagctgcgccgtgaagggcttctacccc | |
| | agcgatatcgccgtggaatgggagagcaacggccagcccgag | |
| | aacaactacaagaccaccccccctgtgctggacagcgacggc | |
| | tcattcttcctggtgtccaagctgacagtggacaagagccgg | |
| | tggcagcagggcaacgtgttcagctgcagcgtgatgcacgag | |
| | gccctgcacaaccattacacccagaagtccctgagcctgagc | |
| | cccggc | |
| Light | LC: | SEQ ID NO: 65 |
| chain A | gacatccagatgacccagagccccagcagcctgagcgccagc | |
| | gtgggcgacagagtgaccatcacctgtcgggccagccagggc | |
| | atccggaactacctggcctggtatcagcagaagcccggcaag | |
| | gcccccaagctgctgatctacgccgccagcacactgcagagc | |
| | ggcgtgcccagcagattcagcggcagcggctccggcaccgac | |
| | ttcaccctgaccatcagcagcctgcagcccgaggacgtggcc | |
| | acctactactgccagcggtacaacagagccccctacaccttc | |
| | ggccagggcaccaaggtggaaatcaagggacagcccaaggct | |
| | gccccctcggtcaccctgttccccccaagcagcgaggaactg | |
| | caggccaacaaggccaccctcgtgtgcctgatcagcgacttc | |
| | taccctggcgccgtgaccgtggcctggaaggccgatagctct | |
| | cccgtgaaggccggcgtggaaaccaccacccccagcaagcag | |
| | agcaacaacaaatacgccgcctccagctacctgagcctgacc | |
| | cccgagcagtggaagtcccaccggtcctacagctgccaggtc | |
| | acacacgagggcagcaccgtggaaaagaccgtggcccccacc | |
| | gagtgcagc | |
| Heavy | HC: | SEQ ID NO: 97 |
| chain B | gaggtgcagctgaaagagtccggccctggactggtggcccct | |
| | ggcggcagcctgagcatcacctgtaccgtgtccggcttcagc | |
| | ctgaccgacagcagcatcaactgggtccgacagccccctggc | |
| | aagggcctggaatggctgggcatgatctggggcgacggccgg | |
| | atcgactacgccgacgccctgaagtcccggctgagcatcagc | |
| | aaggacagcagcaagagccaggtgttcctggaaatgaccagc | |
| | ctgcggaccgacgacaccgccacctactactgcgccagggac | |
| | ggctacttcccctacgccatggatttctggggccagggcacc | |
| | agcgtgaccgtgtccagtcaggtccagctgcagcagagcggc | |
| | cctgagctggtcaagcctggcgccagcgtgaagatcagctgc | |
| | aaggccagcggctacagcttcaccagctactggatccactgg | |
| | atcaagcagcggcctggccagggcctcgagtggatcggaatg | |
| | atcgaccccagcgacggcgagacacggctgaaccagagattc | |
| | cagggcagagccaccctgaccgtggacgagagcaccagcacc | |
| | gcctacatgcagctgcggagccccaccagcgaggacagcgcc | |
| | gtgtactactgcacccggctgaaagaatacggcaactacgac | |
| | agcttctacttcgacgtgtggggagccggcaccctggtcacc | |
| | gtgtctagcgcttccaccaagggccccagcgtgttccctctg | |
| | gcccctagcagcaagagcacatctggcggaacagccgccctg | |
| | ggctgcctggtcaaggactactttcccgagcccgtgaccgtg | |
| | tcctggaactctggtgccctgacaagcggagtgcataccttc | |
| | cctgccgtgctgcagagcagcggcctgtactctctgagcagc | |
| | gtggtcaccgtgccaagcagcagcctgggcacccagacctac | |
| | atctgcaacgtgaaccacaagccctccaacaccaaggtggac | |
| | aagaaggtggaacccaagagctgcgacaagacccacacctgt | |
| | cctccctgtcctgcccctgaactgctgggcggaccctccgtg | |
| | ttcctgttccctccaaagcccaaggataccctgatgatcagc | |
| | cggacccctgaagtgacctgcgtggtggtggacgtgtcccac | |
| | gaggatcccgaagtgaagttcaattggtacgtggacggcgtg | |
| | gaagtgcataacgccaagaccaagcccagagaggaacagtac | |
| | aacagcacctaccgggtggtgtccgtgctgacagtgctgcac | |
| | caggactggctgaacggcaaagagtacaagtgcaaggtgtcc | |
| | aacaaggccctgccagcccctatcgagaaaaccatcagcaag | |
| | gccaagggccagccccgcgagcctcaggtgtacacactgcct | |
| | ccatgccgggacgagctgaccaagaaccaggtgtccctgtgg | |
| | tgcctcgtgaagggcttctacccctccgatatcgccgtggaa | |
| | tgggagagcaacggccagcccgagaacaactacaagaccacc | |
| | cctcccgtgctggacagcgacggctcattcttcctgtacagc | |
| | aagctgaccgtggacaagtcccggtggcagcagggcaacgtg | |
| | ttcagctgctctgtgatgcacgaggccctgcacaaccggttc | |
| | acccagaagtccctgagcctgagccctggc | |
| Light | LC: | SEQ ID NO: 70 |
| chain B | gacatccagatgacccagagccccgccagcctgagcgtgtcc | |
| | gtgggcgataccatcaccctgacctgccacgccagccagaac | |
| | atcgacgtgtggctgagctggttccagcagaagcccggcaac | |
| | atccccaagctgctgatctacaaggccagcaacctgcacacc | |
| | ggcgtgcccagcagattcagcggctctggcagcggcaccggc | |
| | tttaccctgaccatcagcagcctgcagcccgaggatatcgcc | |
| | acctactactgccagcaggcccacagctaccccttcaccttc | |
| | ggcggaggcaccaagctggaaatcaagggcggcagcggcagc | |
| | tccggctctggcggcgatatcgtgctgacccagtctcccgcc | |
| | tccctggccgtgtctctgggccagagagccaccatcagctgc | |
| | cgggccagcgagagcgtggacagctacggccagagctacatg | |
| | cactggtatcagcagaaggccggacagccccctaaactgctc | |
| | atctacctggcctccaacctggaaagcggcgtgcccgccagg | |
| | ttttccggcagcggctccagaaccgacttcaccctgacaatc | |
| | gaccccgtgcaggccgaggacgccgccacatattactgtcag | |
| | cagaacgccgaggacagcagaacctttggcggcggaacaaag | |
| | ctcgagattaagggcggctccggctccagcggatctggcgga | |
| | cgtacggtggccgctccttccgtgttcatcttccctccctcc | |
| | gacgagcagctgaagtccggcaccgcctccgtggtgtgtctg | |
| | ctgaacaacttctaccctcgggaggccaaggtgcagtggaag | |
| | gtggacaacgccctgcagtccggcaactcccaggagtccgtc | |
| | accgagcaggactccaaggacagcacctactccctgtcctcc | |
| | accctgaccctgtccaaggccgactacgagaagcacaaggtg | |
| | tacgcctgtgaggtgacccaccagggcctgtccagccctgtg | |
| | accaagtccttcaaccggggcgagtgc | |

Binding Protein 11 Amino Acid Sequences

| | | |
|---|---|---|
| Heavy | HC: | SEQ ID NO: 60 |
| chain A | EVQLVESGGGLVQPGRSLRLSCAASGFTFDDYAMHWVRQAPG | |
| | KGLEWVSAITWNSGHIDYADSVEGRFTISRDNAKNSLYLQMN | |
| | SLRAEDTAVYYCAKVSYLSTASSLDYWGQGTLVTVSSASTKG | |
| | PSVFPLAPSSKSTSGGTAALGCLVKDYFPEPVTVSWNSGALT | |
| | SGVHTFPAVLQSSGLYSLSSVVTVPSSSLGTQTYICNVNHKP | |
| | SNTKVDKKVEPKSCDKTHTCPPCPAPELLGGPSVFLFPPKPK | |
| | DTLMISRTPEVTCVVVDVSHEDPEVKFNWYVDGVEVHNAKTK | |
| | PREEQYNSTYRVVSVLTVLHQDWLNGKEYKCKVSNKALPAPI | |
| | EKTISKAKGQPREPQVCTLPPSRDELTKNQVSLSCAVKGFYP | |
| | SDIAVEWESNGQPENNYKTTPPVLDSDGSFFLVSKLTVDKSR | |
| | WQQGNVFSCSVMHEALHNHYTQKSLSLSPG | |
| Light | LC: | SEQ ID NO: 71 |
| chain A | DIQMTQSPSSLSASVGDRVTITCRASQGIRNYLAWYQQKPGK | |
| | APKLLIYAASTLQSGVPSRFSGSGSGTDFTLTISSLQPEDVA | |
| | TYYCQQRYNRAPYTFGQGTKVEIKRTVAAPSVFIFPPSDEQLK | |
| | SGTASVVCLLNNFYPREAKVQWKVDNALQSGNSQESVTEQDS | |
| | KDSTYSLSSTLTLSKADYEKHKVYACEVTHQGLSSPVTKSFN | |
| | RGEC | |
| Heavy | HC: | SEQ ID NO: 68 |
| chain B | EVQLKESGPGLVAPGGSLSITCTVSGFSLTDSSINWVRQPPG | |
| | KGLEWLGMIWGDGRIDYADALKSRLSISKDSSKSQVFLEMTSL | |
| | RTDDTATYYCARDGYFPYAMDFWGQGTSVTVSSQVQLQQSGP | |
| | ELVKPGASVKISCKASGYSFTSYWIHWIKQRPGQGLEWIG | |
| | MIDPSDGETRLNQRFQGRATLTVDESTSTAYMQLRSPTSEDS | |
| | AVYYCTRLKEYGNYDSFYFDVWGAGTLVTVSSASTKGPSVFP | |
| | LAPSSKSTSGGTAALGCLVKDYFPEPVTVSWNSGALTSGVHT | |
| | FPAVLQSSGLYSLSSVVTVPSSSLGTQTYICNVNHKPSNTKV | |
| | DKKVEPKSCDKTHTCPPCPAPELLGGPSVFLFPPKPKDTLMI | |
| | SRTPEVTCVVVDVSHEDPEVKFNWYVDGVEVHNAKTKPREEQ | |
| | YNSTYRVVSVLTVLHQDWLNGKEYKCKVSNKALPAPIEKTIS | |
| | KAKGQPREPQVYTLPPCRDELTKNQVSLWCLVKGFYPSDIAV | |
| | EWESNGQPENNYKTTPPVLDSDGSFFLYSKLTVDKSRWQQGN | |
| | VFSCSVMHEALHNRFTQKSLSLSPG | |
| Light | LC: | SEQ ID NO: 69 |
| chain B | DIQMTQSPASLSVSVGDTITLTCHASQNIDVWLSWFQQKPGN | |
| | IPKLLIYKASNLHTGVPSRFSGSGSGTGFTLTISSLQPEDIA | |
| | TYYCQQAHSYPFTFGGGTKLEIKGGSGSSGSGGDIVLTQSPA | |
| | SLAVSLGQRATISCRASESVDSYGQSYMHWYQQKAGQPPKLL | |
| | IYLASNLESGVPARFSGSGSRTDFTLTIDPVQAEDAATYYC | |
| | QQNAEDSRTFGGGTKLEIKGGSGSSGSGGRTVAAPSVFIFPP | |
| | SDEQLKSGTASVVCLLNNFYPREAKVQWKVDNALQSGNSQES | |
| | VTEQDSKDSTYSLSSTLTLSKADYEKHKVYACEVTHQGLSSP | |
| | VTKSFNRGEC | |

Binding Protein 11_Nucleotide Sequences

| | | |
|---|---|---|
| Heavy | HC: | SEQ ID NO: 64 |
| chain A | gaggtgcagctggtggaaagcggcggaggactggtgcagccc | |
| | ggcagaagcctgagactgagctgcgccgccagcggcttcacc | |
| | ttcgacgactacgccatgcactgggtccgccaggcccctggc | |
| | aagggcctggaatgggtgtccgccatcacctggaacagcggc | |
| | cacatcgactacgccgacagcgtggaaggccggttcaccatc | |
| | agccgggacaacgccaagaacagcctgtacctgcagatgaac | |
| | agcctgcgggccgaggacaccgccgtgtactactgcgccaag | |
| | gtgtcctacctgagcaccgccagcagcctggactactggggc | |
| | cagggcaccctggtgacagtgtccagcgcttccaccaagggc | |
| | cccagcgtgttccccctggcccctagcagcaagagcacatct | |
| | ggcggcacagccgccctgggctgcctggtcaaggactacttc | |
| | cccgagcccgtgacagtgtcctggaactctggcgccctgacc | |
| | agcggagtgcataccttccctgccgtgctgcagtccagcggc | |
| | ctgtacagcctgagcagcgtggtcacagtgcccagcagcagc | |
| | ctgggcacccagacctacatctgcaacgtgaaccacaagccc | |
| | agcaacaccaaggtggacaagaaggtggaacccaagagctgc | |
| | gacaagacccacacctgtcccccctgccctgcccctgaactg | |
| | ctgggcggaccctccgtgttcctgttccccccaaagcccaag | |
| | gacaccctgatgatcagccggacccccgaagtgacctgcgtg | |
| | gtggtggacgtgtcccacgaggaccctgaagtgaagttcaat | |
| | tggtacgtggacggcgtggaagtgcataacgccaagaccaag | |
| | cccagagaggaacagtacaacagcacctaccgggtggtgtcc | |
| | gtgctgaccgtgctgcaccaggactggctgaacggcaaagag | |
| | tacaagtgcaaggtgtccaacaaggccctgcctgcccccatc | |
| | gagaaaaccatcagcaaggccaagggccagcctagagagccc | |
| | caagtctgcaccctgccccccagcagagatgagctgaccaag | |
| | aaccaggtgtccctgagctgcgccgtgaagggcttctacccc | |
| | agcgatatcgccgtggaatgggagagcaacggccagcccgag | |
| | aacaactacaagaccaccccccctgtgctggacagcgacggc | |
| | tcattcttcctggtgtccaagctgacagtggacaagagccgg | |
| | tggcagcagggcaacgtgttcagctgcagcgtgatgcacgag | |
| | gccctgcacaaccattacacccagaagtccctgagcctgagc | |
| | cccggc | |
| Light | LC: | SEQ ID NO: 72 |
| chain A | gacatccagatgacccagagccccagcagcctgagcgccagc | |
| | gtgggcgacagagtgaccatcacctgtcgggccagccagggc | |
| | atccggaactacctggcctggtatcagcagaagcccggcaag | |
| | gcccccaagctgctgatctacgccgccagcacactgcagagc | |
| | ggcgtgcccagcagattcagcggcagcggctccggcaccgac | |
| | ttcaccctgaccatcagcagcctgcagcccgaggacgtggcc | |
| | acctactactgccagcggtacaacagagccccctacaccttc | |
| | ggccagggcaccaaggtggaaatcaagcgtacggtggccgct | |
| | ccttccgtgttcatcttccctccctccgacgagcagctgaag | |
| | tccggcaccgcctccgtggtgtgtctgctgaacaacttctac | |
| | cctcgggaggccaaggtgcagtggaaggtggacaacgccctg | |
| | cagtccggcaactcccaggagtccgtcaccgagcaggactcc | |
| | aaggacagcacctactccctgtcctccaccctgaccctgtcc | |
| | aaggccgactacgagaagcacaaggtgtacgcctgtgaggtg | |
| | acccaccagggcctgtccagccctgtgaccaagtccttcaac | |
| | cggggcgagtgc | |
| Heavy | HC: | SEQ ID NO: 97 |
| chain B | gaggtgcagctgaaagagtccggccctggactggtggcccct | |
| | ggcggcagcctgagcatcacctgtaccgtgtccggcttcagc | |
| | ctgaccgacagcagcatcaactgggtccgacagccccctggc | |
| | aagggcctggaatggctgggcatgatctggggcgacggccgg | |
| | atcgactacgccgacgccctgaagtcccggctgagcatcagc | |
| | aaggacagcagcaagagccaggtgttcctggaaatgaccagc | |
| | ctgcggaccgacgacaccgccacctactactgcgccagggac | |
| | ggctacttcccctacgccatggatttctggggccagggcacc | |
| | agcgtgaccgtgtccagtcaggtccagctgcagcagagcggc | |
| | cctgagctggtcaagcctggcgccagcgtgaagatcagctgc | |
| | aaggccagcggctacagcttcaccagctactggatccactgg | |
| | atcaagcagcggcctggccagggcctcgagtggatcggaatg | |
| | atcgaccccagcgacggcgagacacggctgaaccagagattc | |
| | cagggcagagccaccctgaccgtggacgagagcaccagcacc | |
| | gcctacatgcagctgcggagccccaccagcgaggacagcgcc | |
| | gtgtactactgcacccggctgaaagaatacggcaactacgac | |
| | agcttctacttcgacgtgtggggagccggcaccctggtcacc | |
| | gtgtctagcgcttccaccaagggccccagcgtgttccctctg | |
| | gcccctagcagcaagagcacatctggcggaacagccgccctg | |
| | ggctgcctggtcaaggactactacccgagcccgtgaccgtgt | |
| | cctggaactctggtgccctgacaagcggagtgcataccttcc | |
| | ctgccgtgctgcagagcagcggcctgtactctctgagcagcg | |
| | tggtcaccgtgccaagcagcagcctgggcacccagacctaca | |
| | tctgcaacgtgaaccacaagccctccaacaccaaggtggaca | |
| | agaaggtggaacccaagagctgcgacaagacccacacctgtc | |
| | ctccctgtcctgcccctgaactgctgggcggaccctccgtgt | |
| | tcctgttccctccaaagcccaaggataccctgatgatcagcc | |
| | ggacccctgaagtgacctgcgtggtggtggacgtgtcccacg | |
| | aggatcccgaagtgaagttcaattggtacgtggacggcgtgg | |
| | aagtgcataacgccaagaccaagcccagagaggaacagtaca | |
| | acagcacctaccgggtggtgtccgtgctgacagtgctgcacc | |
| | aggactggctgaacggcaaagagtacaagtgcaaggtgtcca | |
| | acaaggccctgccagcccctatcgagaaaaccatcagcaagg | |
| | ccaagggccagccccgcgagcctcaggtgtacacactgcctc | |
| | catgccgggacgagctgaccaagaaccaggtgtccctgtggt | |
| | gcctcgtgaagggcttctacccctccgatatcgccgtggaat | |
| | gggagagcaacggccagcccgagaacaactacaagaccaccc | |
| | ctcccgtgctggacagcgacggctcattcttcctgtacagca | |
| | agctgaccgtggacaagtcccggtggcagcagggcaacgtgt | |
| | tcagctgctctgtgatgcacgaggccctgcacaaccggttca | |
| | cccagaagtccctgagcctgagccctggc | |
| Light | LC: | SEQ ID NO: 70 |
| chain B | gacatccagatgacccagagccccgccagcctgagcgtgtcc | |
| | gtgggcgataccatcaccctgacctgccacgccagccagaac | |
| | atcgacgtgtggctgagctggttccagcagaagcccggcaac | |
| | atccccaagctgctgatctacaaggccagcaacctgcacacc | |
| | ggcgtgcccagcagattcagcggctctggcagcggcaccggc | |
| | tttaccctgaccatcagcagcctgcagcccgaggatatcgcc | |
| | acctactactgccagcaggcccacagctaccccttcaccttc | |
| | ggcggaggcaccaagctggaaatcaagggcggcagcggcagc | |
| | tccggctctggcggcgatatcgtgctgacccagtctcccgcc | |
| | tccctggccgtgtctctgggccagagagccaccatcagctgc | |
| | cgggccagcgagagcgtggacagctacggccagagctacatg | |
| | cactggtatcagcagaaggccggacagccccctaaactgctc | |
| | atctacctggcctccaacctggaaagcggcgtgcccgccagg | |
| | ttttccggcagcggctccagaaccgacttcaccctgacaatc | |
| | gaccccgtgcaggccgaggacgccgccacatattactgtcag | |
| | cagaacgccgaggacagcagaacctttggcggcggaacaaag | |
| | ctcgagattaagggcggctccggctccagcggatctggcgga | |
| | cgtacggtggccgctccttccgtgttcatcttccctccctcc | |
| | gacgagcagctgaagtccggcaccgcctccgtggtgtgtctg | |
| | ctgaacaacttctaccctcgggaggccaaggtgcagtggaag | |
| | gtggacaacgccctgcagtccggcaactcccaggagtccgtc | |
| | accgagcaggactccaaggacagcacctactccctgtcctcc | |
| | accctgaccctgtccaaggccgactacgagaagcacaaggtg | |
| | tacgcctgtgaggtgacccaccagggcctgtccagccctgtg | |
| | accaagtccttcaaccggggcgagtgc | |

Binding Protein 12 Amino Acid Sequences

| | | |
|---|---|---|
| Heavy | HC: | SEQ ID NO: 73 |
| chain A | EVQLKESGPGLVAPGGSLSITCTVSGFSLTDSSINWVRQPPG | |
| | KGLEWLGMIWGDGRIDYADALKSRLSISKDSSKSQVFLEMTSL | |
| | RTDDTATYYCARDGYFPYAMDFWGQGTSVTVSSASTKGPSVF | |
| | PLAPSSKSTSGGTAALGCLVKDYFPEPVTVSWNSGALTSGVH | |
| | TFPAVLQSSGLYSLSSVVTVPSSSLGTQTYICNVNHKPSNTK | |
| | VDKKVEPKSCDKTHTCPPCPAPELLGGPSVFLFPPKPKDTLM | |
| | ISRTPEVTCVVVDVSHEDPEVKFNWYVDGVEVHNAKTKPREE | |
| | QYNSTYRVVSVLTVLHQDWLNGKEYKCKVSNKALPAPIEKTI | |
| | SKAKGQPREPQVCTLPPSRDELTKNQVSLSCAVKGFYPSDIA | |
| | VEWESNGQPENNYKTTPPVLDSDGSFFLVSKLTVDKSRWQQG | |
| | NVFSCSVMHEALHNHYTQKSLSLSPG | |
| Light | LC: | SEQ ID NO: 74 |
| chain A | DIVLTQSPASLAVSLGQRATISCRASESVDSYGQSYMHWYQQ | |
| | KAGQPPKLLIYLASNLESGVPARFSGSGSRTDFTLTIDPVQA | |
| | EDAATYYCQQNAEDSRTFGGGTKLEIKGQPKAAPSVTLFPPS | |
| | SEELQANKATLVCLISDFYPGAVTVAWKADSSPVKAGVETTT | |
| | PSKQSNNKYAASSYLSLTPEQWKSHRSYSCQVTHEGSTVEKT | |
| | VAPTECS | |
| Heavy | HC: | SEQ ID NO: 75 |
| chain B | QVQLQQSGPELVKPGASVKISCKASGYSFTSYWIHWIKQRPG | |
| | QGLEWIGMIDPSDGETRLNQUQGRATLTVDESTSTAYMQLRS | |
| | PTSEDSAVYYCTRLKEYGNYDSFYFDVWGAGTLVTVSSEVQL | |
| | VESGGGLVQPGRSLRLSCAASGFTFDDYAMHWVRQAPGKGLE | |
| | WVSAITWNSGHIDYADSVEGRFTISRDNAKNSLYLQMNSLRA | |
| | EDTAVYYCAKVSYLSTASSLDYWGQGTLVTVSSASTKGPSVF | |
| | PLAPSSKSTSGGTAALGCLVKDYFPEPVTVSWNSGALTSGVH | |
| | TFPAVLQSSGLYSLSSVVTVPSSSLGTQTYICNVNHKPSNTK | |
| | VDKKVEPKSCDKTHTCPPCPAPELLGGPSVFLFPPKPKDTLM | |
| | ISRTPEVTCVVVDVSHEDPEVKFNWYVDGVEVHNAKTKPREE | |
| | QYNSTYRVVSVLTVLHQDWLNGKEYKCKVSNKALPAPIEKTI | |
| | SKAKGQPREPQVYTLPPCRDELTKNQVSLWCLVKGFYPSDIA | |
| | VEWESNGQPENNYKTTPPVLDSDGSFFLYSKLTVDKSRWQQG | |
| | NVFSCSVMHEALHNRFTQKSLSLSPG | |
| Light | LC: | SEQ ID NO: 76 |
| chain B | DIQMTQSPSSLSASVGDRVTITCRASQGIRNYLAWYQQKPGK | |
| | APKLLIYAASTLQSGVPSRFSGSGSGTDFTLTISSLQPEDVA | |
| | TYYCQRYNRAPYTFGQGTKVEIKGGSGSSGSGGDIQMTQSPA | |
| | SLSVSVGDTITLTCHASQNIDVWLSWFQQKPGNIPKLLIY | |
| | KASNLHTGVPSRFSGSGSGTGFTLTISSLQPEDIATYYC | |
| | QQAHSYPFTFGGGTKLEIKGGSGSSGSGGRTVAAPSVFIFPP | |
| | SDEQLKSGTASVVCLLNNFYPREAKVQWKVDNALQSGNSQES | |
| | VTEQDSKDSTYSLSSTLTLSKADYEKHKVYACEVTHQGLSSP | |
| | VTKSFNRGEC | |

Binding Protein 12 Nucleotide Sequences

| | | |
|---|---|---|
| Heavy | HC: | SEQ ID NO: 77 |
| chain A | gaggtgcagctgaaggagagcggccccggcctggtggccccc | |
| | ggcggcagcctgagcatcacctgcaccgtgagcggcttcagc | |
| | ctgaccgacagcagcatcaactgggtgcgccagccccccggc | |
| | aagggcctggagtggctgggcatgatctggggcgacggccgc | |
| | atcgactacgccgacgccctgaagagccgcctgagcatcagc | |
| | aaggacagcagcaagagccaggtgttcctggagatgaccagc | |
| | ctgcgcaccgacgacaccgccacctactactgcgcccgcgac | |
| | ggctacttcccctacgccatggacttctggggccagggcacc | |
| | agcgtgaccgtgagcagcgccagcaccaagggccccagcgtg | |
| | ttccccctggcccctagcagcaagagcacatctggcggcaca | |
| | gccgccctgggctgcctggtcaaggactacttccccgagccc | |
| | gtgacagtgtcctggaactctggcgccctgaccagcggagtg | |
| | cataccttccctgccgtgctgcagtccagcggcctgtacagc | |
| | ctgagcagcgtggtcacagtgcccagcagcagcctgggcacc | |
| | cagacctacatctgcaacgtgaaccacaagcccagcaacacc | |
| | aaggtggacaagaaggtggaacccaagagctgcgacaagacc | |
| | cacacctgtcccccctgccctgcccctgaactgctgggcgga | |
| | ccctccgtgttcctgttccccccaaagcccaaggacaccctg | |
| | atgatcagccggacccccgaagtgacctgcgtggtggtggac | |
| | gtgtcccacgaggaccctgaagtgaagttcaattggtacgtg | |
| | gacggcgtggaagtgcataacgccaagaccaagcccagagag | |
| | gaacagtacaacagcacctaccgggtggtgtccgtgctgacc | |
| | gtgctgcaccaggactggctgaacggcaaagagtacaagtgc | |
| | aaggtgtccaacaaggccctgcctgcccccatcgagaaaacc | |
| | atcagcaaggccaagggccagcctagagagccccaagtctgc | |
| | accctgccccccagcagagatgagctgaccaagaaccaggtg | |
| | tccctgagctgcgccgtgaagggcttctaccccagcgatatc | |
| | gccgtggaatgggagagcaacggccagcccgagaacaactac | |
| | aagaccaccccccctgtgctggacagcgacggctcattcttc | |
| | ctggtgtccaagctgacagtggacaagagccggtggcagcag | |
| | ggcaacgtgttcagctgcagcgtgatgcacgaggccctgcac | |
| | aaccattacacccagaagtccctgagcctgagccccggc | |
| Light | LC: | SEQ ID NO: 78 |
| chain A | gacatcgtgctgacccagagccctgccagcctggccgtgtct | |
| | ctgggccagagagccaccatcagctgccgggccagcgagagc | |
| | gtggacagctacggccagagctacatgcactggtatcagcag | |
| | aaggccggccagccccccaagctgctgatctacctggccagc | |
| | aacctggaaagcggcgtgcccgccagattcagcggcagcggc | |
| | agcagaaccgacttcaccctgaccatcgaccccgtgcaggcc | |
| | gaggacgccgccacctactactgccagcagaacgccgaggac | |
| | agccggaccttcggcggaggcaccaagctggaaatcaaggga | |
| | cagcccaaggctgccccctcggtcaccctgttccccccaagc | |
| | agcgaggaactgcaggccaacaaggccaccctcgtgtgcctg | |
| | atcagcgacttctaccctggcgccgtgaccgtggcctggaag | |
| | gccgatagctctcccgtgaaggccggcgtggaaaccaccacc | |
| | cccagcaagcagagcaacaacaaatacgccgccagcagctac | |
| | ctgagcctgacccccgagcagtggaagtcccaccggtcctac | |
| | agctgccaggtcacacacgagggcagcaccgtggaaaagacc | |
| | gtggcccccaccgagtgcagc | |
| Heavy | HC: | SEQ ID NO: 79 |
| chain B | caggtgcagctgcagcagagcggccctgagctggtcaagcct | |
| | ggcgccagcgtgaagatcagctgcaaggccagcggctacagc | |
| | ttcaccagctactggatccactggatcaagcagcggcctggc | |
| | cagggcctggaatggatcggcatgatcgaccccagcgacggc | |
| | gagacacggctgaaccagagattccagggcagagccaccctg | |
| | accgtggacgagagcaccagcaccgcctacatgcagctgcgg | |
| | agccccaccagcgaggacagcgccgtgtactactgcacccgg | |
| | ctgaaagagtacggcaactacgacagatctacttcgacgtgt | |
| | ggggagccggcaccctggtcaccgtgtccagcgaagtgcagc | |
| | tggtggaaagcggcggaggcctggtgcagcccggcagaagcc | |
| | tgagactgagctgcgccgccagcggcttcaccttcgacgact | |
| | acgccatgcactgggtccgacaggcccctggcaaaggactgg | |
| | aatgggtgtccgccatcacctggaacagcggccacatcgact | |
| | acgccgacagcgtggaaggccggttcaccatcagccgggaca | |
| | acgccaagaacagcctgtacctgcagatgaacagcctgcggg | |
| | ccgaggataccgccgtgtattattgcgccaaggtgtcctacc | |
| | tgagcaccgccagcagcctggactactggggccagggcaccc | |
| | tcgtgacagtgtcctccgcttccaccaagggccccagcgtgt | |
| | tccctctggcccctagcagcaagagcacatctggcggaacag | |
| | ccgccctgggctgcctggtcaaggactactttcccgagcccg | |
| | tgaccgtgtcctggaactctggtgccctgacaagcggagtgc | |
| | ataccttccctgccgtgctgcagagcagcggcctgtactctc | |
| | tgagcagcgtggtcaccgtgccaagcagcagcctgggcaccc | |
| | agacctacatctgcaacgtgaaccacaagccctccaacacca | |
| | aggtggacaagaaggtggaacccaagagctgcgacaagaccc | |
| | acacctgtcctccctgtcctgcccctgaactgctgggcggac | |
| | cctccgtgttcctgttccctccaaagcccaaggataccctga | |
| | tgatcagccggacccctgaagtgacctgcgtggtggtggacg | |
| | tgtcccacgaggatcccgaagtgaagttcaattggtacgtgg | |
| | acggcgtggaagtgcataacgccaagaccaagcccagagagg | |
| | aacagtacaacagcacctaccgggtggtgtccgtgctgacag | |
| | tgctgcaccaggactggctgaacggcaaagagtacaagtgca | |
| | aggtgtccaacaaggccctgccagcccctatcgagaaaacca | |
| | tcagcaaggccaagggccagccccgcgagcctcaggtgtaca | |
| | cactgcctccatgccgggacgagctgaccaagaaccaggtgt | |
| | ccctgtggtgcctcgtgaagggatctacccctccgatatcgc | |
| | cgtggaatgggagagcaacggccagcccgagaacaactacaa | |
| | gaccacccctcccgtgctggacagcgacggctcattcttcct | |
| | gtacagcaagctgaccgtggacaagtcccggtggcagcaggg | |
| | caacgtgttcagctgctctgtgatgcacgaggccctgcacaa | |
| | ccggttcacccagaagtccctgagcctgagccctggc | |
| Light | LC: | SEQ ID NO: 80 |
| chain B | gacatccagatgacccagagccccagcagcctgagcgccagc | |
| | gtgggcgacagagtgaccatcacctgtcgggccagccagggc | |
| | atccggaactacctggcctggtatcagcagaagcccggcaag | |
| | gcccccaagctgctgatctacgccgccagcacactgcagagc | |
| | ggcgtgcccagcagattcagcggcagcggctccggcaccgac | |
| | ttcaccctgaccatcagcagcctgcagcccgaggacgtggcc | |
| | acctactactgccagcggtacaacagagccccctacaccttc | |
| | ggccagggcaccaaggtggaaatcaagggcggctctggcagc | |
| | tccggcagcggcggagacattcagatgacacagtcccccgcc | |
| | agcctgtccgtgtccgtgggcgataccatcaccctgacatgc | |
| | cacgccagccagaacatcgacgtgtggctgagctggttccag | |
| | cagaaacctggcaacatccctaagctgctcatctataaggcc | |
| | agcaacctgcacacaggcgtgccctccagattctccggctct | |
| | ggctctggcaccggctttacactgacaatcagttctctgcag | |
| | cctgaggatatcgccacatattactgtcagcaggcccacagc | |
| | taccctttcaccttcggaggcggcaccaagctcgagattaag | |
| | ggcggaagcggctcctccggctccggcggacgtacggtggcc | |
| | gctccttccgtgttcatcttccctccctccgacgagcagctg | |
| | aagtccggcaccgcctccgtggtgtgtctgctgaacaacttc | |
| | taccctcgggaggccaaggtgcagtggaaggtggacaacgcc | |
| | ctgcagtccggcaactcccaggagtccgtcaccgagcaggac | |
| | tccaaggacagcacctactccctgtcctccaccctgaccctg | |
| | tccaaggccgactacgagaagcacaaggtgtacgcctgtgag | |
| | gtgacccaccagggcctgtccagccctgtgaccaagtccttc | |
| | aaccggggcgagtgc | |

Binding Protein 13 Amino Acid Sequences

| | | |
|---|---|---|
| Heavy | HC: | SEQ ID NO: 73 |
| chain A | EVQLKESGPGLVAPGGSLSITCTVSGFSLTDSSINWVRQPP | |
| | GKGLEWLGMIWGDGRIDYADALKSRLSISKDSSKSQVFLEMT | |
| | SLRTDDTATYYCARDGYFPYAMDFWGQGTSVTVSSASTKGPS | |
| | VFPLAPSSKSTSGGTAALGCLVKDYFPEPVTVSWNSGALTSG | |
| | VHTFPAVLQSSGLYSLSSVVTVPSSSLGTQTYICNVNHKPSN | |
| | TKVDKKVEPKSCDKTHTCPPCPAPELLGGPSVFLFPPKPKDT | |
| | LMISRTPEVTCVVVDVSHEDPEVKFNWYVDGVEVHNAKTKPR | |
| | EEQYNSTYRVVSVLTVLHQDWLNGKEYKCKVSNKALPAPIEK | |
| | TISKAKGQPREPQVCTLPPSRDELTKNQVSLSCAVKGFYPSD | |
| | IAVEWESNGQPENNYKTTPPVLDSDGSFFLVSKLTVDKSRWQ | |
| | QGNVFSCSVMHEALHNHYTQKSLSLSPG | |
| Light | LC: | SEQ ID NO: 74 |
| chain A | DIVLTQSPASLAVSLGQRATISCRASESVDSYGQSYMHWYQQ | |
| | KAGQPPKLLIYLASNLESGVPARFSGSGSRTDFTLTIDPVQA | |
| | EDAATYYCQQNAEDSRTFGGGTKLEIKGQPKAAPSVTLFPPS | |
| | SEELQANKATLVCLISDFYPGAVTVAWKADSSPVKAGVETTT | |
| | PSKQSNNKYAASSYLSLTPEQWKSHRSYSCQVTHEGSTVEKT | |
| | VAPTECS | |
| Heavy | HC: | SEQ ID NO: 81 |
| chain B | EVQLVESGGGLVQPGRSLRLSCAASGFTFDDYAMHWVRQAPG | |
| | KGLEWVSAITWNSGHIDYADSVEGRFTISRDNAKNSLYLQMN | |
| | SLRAEDTAVYYCAKVSYLSTASSLDYWGQGTLVTVSSQVQLQ | |
| | QSGPELVKPGASVKISCKASGYSFTSYWIHWIKQRPGQGLEW | |
| | IGMIDPSDGETRLNQRFQGRATLTVDESTSTAYMQLRSPTSE | |
| | DSAVYYCTRLKEYGNYDSFYFDVWGAGTLVTVSSASTKGPSV | |
| | FPLAPSSKSTSGGTAALGCLVKDYFPEPVTVSWNSGALTSGV | |
| | HTFPAVLQSSGLYSLSSVVTVPSSSLGTQTYICNVNHKPSNT | |
| | KVDKKVEPKSCDKTHTCPPCPAPELLGGPSVFLFPPKPKDTL | |
| | MISRTPEVTCVVVDVSHEDPEVKFNWYVDGVEVHNAKTKPRE | |
| | EQYNSTYRVVSVLTVLHQDWLNGKEYKCKVSNKALPAPIEKT | |
| | ISKAKGQPREPQVYTLPPCRDELTKNQVSLWCLVKGFYPSDI | |
| | AVEWESNGQPENNYKTTPPVLDSDGSFFLYSKLTVDKSRWQQ | |
| | GNVFSCSVMHEALHNRFTQKSLSLSPG | |
| Light | LC: | SEQ ID NO: 82 |
| chain B | DIQMTQSPASLSVSVGDTITLTCHASQNIDVWLSWFQQKPGN | |
| | IPKLLIYKASNLHTGVPSRFSGSGSGTGFTLTISSLQPEDIA | |
| | TYYCQQAHSYPFTFGGGTKLEIKGGSGSSGSGGDIQMTQSPS | |
| | SLSASVGDRVTITCRASQGIRNYLAWYQQKPGKAPKLLIY | |
| | AASTLQSGVPSRFSGSGSGTDFTLTISSLQPEDVATYYC | |
| | QRYNRAPYTFGQGTKVEIKGGSGSSGSGGRTVAAPSVFIFPP | |
| | SDEQLKSGTASVVCLLNNFYPREAKVQWKVDNALQSGNSQES | |
| | VTEQDSKDSTYSLSSTLTLSKADYEKHKVYACEVTHQGLSSP | |
| | VTKSFNRGEC | |

Binding Protein 13_Nucleotide Sequences

| | | |
|---|---|---|
| Heavy | HC: | SEQ ID NO: 77 |
| chain A | gaggtgcagctgaaggagagcggccccggcctggtggccccc | |
| | ggcggcagcctgagcatcacctgcaccgtgagcggcttcagc | |
| | ctgaccgacagcagcatcaactgggtgcgccagccccccggc | |
| | aagggcctggagtggctgggcatgatctggggcgacggccgc | |
| | atcgactacgccgacgccctgaagagccgcctgagcatcagc | |
| | aaggacagcagcaagagccaggtgttcctggagatgaccagc | |
| | ctgcgcaccgacgacaccgccacctactactgcgcccgcgac | |
| | ggctacttcccctacgccatggacttctggggccagggcacc | |
| | agcgtgaccgtgagcagcgccagcaccaagggccccagcgtg | |
| | ttccccctggcccctagcagcaagagcacatctggcggcaca | |
| | gccgccctgggctgcctggtcaaggactacttccccgagccc | |
| | gtgacagtgtcctggaactctggcgccctgaccagcggagtg | |
| | cataccttccctgccgtgctgcagtccagcggcctgtacagc | |
| | ctgagcagcgtggtcacagtgcccagcagcagcctgggcacc | |
| | cagacctacatctgcaacgtgaaccacaagcccagcaacacc | |
| | aaggtggacaagaaggtggaacccaagagctgcgacaagacc | |
| | cacacctgtcccccctgccctgcccctgaactgctgggcgga | |
| | ccctccgtgttcctgttccccccaaagcccaaggacaccctg | |
| | atgatcagccggacccccgaagtgacctgcgtggtggtggac | |
| | gtgtcccacgaggaccctgaagtgaagttcaattggtacgtg | |
| | gacggcgtggaagtgcataacgccaagaccaagcccagagag | |
| | gaacagtacaacagcacctaccgggtggtgtccgtgctgacc | |
| | gtgctgcaccaggactggctgaacggcaaagagtacaagtgc | |
| | aaggtgtccaacaaggccctgcctgcccccatcgagaaaacc | |
| | atcagcaaggccaagggccagcctagagagccccaagtctgc | |
| | accctgccccccagcagagatgagctgaccaagaaccaggtg | |
| | tccctgagctgcgccgtgaagggcttctaccccagcgatatc | |
| | gccgtggaatgggagagcaacggccagcccgagaacaactac | |
| | aagaccaccccccctgtgctggacagcgacggctcattcttc | |
| | ctggtgtccaagctgacagtggacaagagccggtggcagcag | |
| | ggcaacgtgttcagctgcagcgtgatgcacgaggccctgcac | |
| | aaccattacacccagaagtccctgagcctgagccccggc | |
| Light | LC: | SEQ ID NO: 78 |
| chain A | gacatcgtgctgacccagagccctgccagcctggccgtgtct | |
| | ctgggccagagagccaccatcagctgccgggccagcgagagc | |
| | gtggacagctacggccagagctacatgcactggtatcagcag | |
| | aaggccggccagccccccaagctgctgatctacctggccagc | |
| | aacctggaaagcggcgtgcccgccagattcagcggcagcggc | |
| | agcagaaccgacttcaccctgaccatcgaccccgtgcaggcc | |
| | gaggacgccgccacctactactgccagcagaacgccgaggac | |
| | agccggaccttcggcggaggcaccaagctggaaatcaaggga | |
| | cagcccaaggctgccccctcggtcaccctgttccccccaagc | |
| | agcgaggaactgcaggccaacaaggccaccctcgtgtgcctg | |
| | atcagcgacttctaccctggcgccgtgaccgtggcctggaag | |
| | gccgatagctctcccgtgaaggccggcgtggaaaccaccacc | |
| | cccagcaagcagagcaacaacaaatacgccgccagcagctac | |
| | ctgagcctgacccccgagcagtggaagtcccaccggtcctac | |
| | agctgccaggtcacacacgagggcagcaccgtggaaaagacc | |
| | gtggcccccaccgagtgcagc | |
| Heavy | HC: | SEQ ID NO: 83 |
| chain B | gaggtgcagctggtggaaagcggcggaggactggtgcagccc | |
| | ggcagaagcctgagactgagctgcgccgccagcggcttcacc | |
| | ttcgacgactacgccatgcactgggtccgacaggcccctggc | |
| | aagggcctggaatgggtgtccgccatcacctggaacagcggc | |
| | cacatcgactacgccgacagcgtggaaggccggttcaccatc | |
| | agccgggacaacgccaagaacagcctgtacctgcagatgaac | |
| | agcctgcgggccgaggacaccgccgtgtactactgcgccaag | |
| | gtgtcctacctgagcaccgccagcagcctggactactggggc | |
| | cagggcaccctggtcaccgtgtccagtcaggtccagctgcag | |
| | cagagcggccctgagctggtcaagcctggcgccagcgtgaag | |
| | atcagctgcaaggccagcggctacagcttcaccagctactgg | |
| | atccactggatcaagcagcggcctggccagggcctcgagtgg | |
| | atcggcatgatcgaccccagcgacggcgagacacggctgaac | |
| | cagagattccagggcagagccaccctgaccgtggacgagagc | |
| | accagcaccgcctacatgcagctgcggagccccaccagcgag | |
| | gatagcgccgtgtattattgcacccggctgaaagagtacggc | |
| | aactacgacagcttctacttcgacgtgtggggagccggcacc | |
| | ctcgtgacagtgtcctccgcttccaccaagggccccagcgtg | |
| | ttccctctggcccctagcagcaagagcacatctggcggaaca | |
| | gccgccctgggctgcctggtcaaggactactucccgagcccg | |
| | tgaccgtgtcctggaactctggtgccctgacaagcggagtgc | |
| | ataccttccctgccgtgctgcagagcagcggcctgtactctc | |
| | tgagcagcgtggtcaccgtgccaagcagcagcctgggcaccc | |
| | agacctacatctgcaacgtgaaccacaagccctccaacacca | |
| | aggtggacaagaaggtggaacccaagagctgcgacaagaccc | |
| | acacctgtcctccctgtcctgcccctgaactgctgggcggac | |
| | cctccgtgttcctgttccctccaaagcccaaggataccctga | |
| | tgatcagccggacccctgaagtgacctgcgtggtggtggacg | |
| | tgtcccacgaggatcccgaagtgaagttcaattggtacgtgg | |
| | acggcgtggaagtgcataacgccaagaccaagcccagagagg | |
| | aacagtacaacagcacctaccgggtggtgtccgtgctgacag | |
| | tgctgcaccaggactggctgaacggcaaagagtacaagtgca | |
| | aggtgtccaacaaggccctgccagcccctatcgagaaaacca | |
| | tcagcaaggccaagggccagccccgcgagcctcaggtgtaca | |
| | cactgcctccatgccgggacgagctgaccaagaaccaggtgt | |
| | ccctgtggtgcctcgtgaagggcttctacccctccgatatcg | |
| | ccgtggaatgggagagcaacggccagcccgagaacaactaca | |
| | agaccacccctcccgtgctggacagcgacggctcattcttcc | |
| | tgtacagcaagctgaccgtggacaagtcccggtggcagcagg | |
| | gcaacgtgttcagctgctctgtgatgcacgaggccctgcaca | |
| | accggttcacccagaagtccctgagcctgagccctggc | |
| Light | LC: | SEQ ID NO: 84 |
| chain B | gacatccagatgacccagagccccgccagcctgagcgtgtcc | |
| | gtgggcgataccatcaccctgacctgccacgccagccagaac | |
| | atcgacgtgtggctgagctggttccagcagaagcccggcaac | |
| | atccccaagctgctgatctacaaggccagcaacctgcacacc | |
| | ggcgtgcccagcagattcagcggctctggcagcggcaccggc | |
| | tttaccctgaccatcagcagcctgcagcccgaggatatcgcc | |
| | acctactactgccagcaggcccacagctaccccttcaccttc | |
| | ggcggaggcaccaagctggaaatcaagggcggcagcggcagc | |
| | tccggcagcggcggagacattcagatgacacagtcccccagc | |
| | agcctgtccgccagcgtgggcgacagagtgaccatcacctgt | |
| | cgggccagccagggcatccggaactacctggcctggtatcag | |
| | cagaaacctggcaaggcccctaaactgctcatctacgccgcc | |
| | agcacactgcagtctggcgtgccctccagattctccggaagc | |
| | ggctccggcaccgatttcaccctgacaatctcatctctgcag | |
| | cctgaggacgtggccacatattactgccagagatacaacaga | |
| | gccccctacacctttggccagggcaccaaggtcgagattaag | |
| | ggcggatccggctccagcggcagcggaggacgtacggtggcc | |
| | gctccttccgtgttcatcttccctccctccgacgagcagctg | |
| | aagtccggcaccgcctccgtggtgtgtctgctgaacaacttc | |
| | taccctcgggaggccaaggtgcagtggaaggtggacaacgcc | |
| | ctgcagtccggcaactcccaggagtccgtcaccgagcaggac | |
| | tccaaggacagcacctactccctgtcctccaccctgaccctg | |
| | tccaaggccgactacgagaagcacaaggtgtacgcctgtgag | |
| | gtgacccaccagggcctgtccagccctgtgaccaagtccttc | |
| | aaccggggcgagtgc | |

Binding Protein 14 Amino Acid Sequences

| | | |
|---|---|---|
| Heavy | HC: | SEQ ID NO: 85 |
| chain A | QVQLQQSGPELVKPGASVKISCKASGYSFTSYWIHWIKQRPG | |
| | QGLEWIGMIDPSDGETRLNQRFQGRATLTVDESTSTAYMQLR | |
| | SPTSEDSAVYYCTRLKEYGNYDSFYFDVWGAGTLVTVSSAST | |
| | KGPSVFPLAPSSKSTSGGTAALGCLVKDYFPEPVTVSWNSGA | |
| | LTSGVHTFPAVLQSSGLYSLSSVVTVPSSSLGTQTYICNVNH | |
| | KPSNTKVDKKVEPKSCDKTHTCPPCPAPELLGGPSVFLFPPK | |
| | PKDTLMISRTPEVTCVVVDVSHEDPEVKFNWYVDGVEVHNAK | |
| | TKPREEQYNSTYRVVSVLTVLHQDWLNGKEYKCKVSNKALPA | |
| | PIEKTISKAKGQPREPQVCTLPPSRDELTKNQVSLSCAVKGF | |
| | YPSDIAVEWESNGQPENNYKTTPPVLDSDGSFFLVSKLTVDK | |
| | SRWQQGNVFSCSVMHEALHNHYTQKSLSLSPG | |
| Light | LC: | SEQ ID NO: 86 |
| chain A | DIQMTQSPASLSVSVGDTITLTCHASQNIDVWLSWFQQKPGN | |
| | IPKLLIYKASNLHTGVPSRFSGSGSGTGFTLTISSLQPEDIA | |
| | TYYCQQAHSYPFTFGGGTKLEIKGQPKAAPSVTLFPPSSEEL | |
| | QANKATLVCLISDFYPGAVTVAWKADSSPVKAGVETTTPSKQ | |
| | SNNKYAASSYLSLTPEQWKSHRSYSCQVTHEGSTVEKTVAPT | |
| | ECS | |
| Heavy | HC: | SEQ ID NO: 87 |
| chain B | EVQLKESGPGLVAPGGSLSITCTVSGFSLTDSSINWVRQPP | |
| | GKGLEWLGMIWGDGRIDYADALKSRLSISKDSSKSQVFLEMT | |
| | SLRTDDTATYYCARDGYFPYAMDFWGQGTSVTVSSEVQLVESG | |
| | GGLVQPGRSLRLSCAASGFTFDDYAMHWVRQAPGKGLEWVS | |
| | AITWNSGHIDYADSVEGRFTISRDNAKNSLYLQMNSLRAEDTA | |
| | VYYCAKVSYLSTASSLDYWGQGTLVTVSSASTKGPSVFPLAP | |
| | SSKSTSGGTAALGCLVKDYFPEPVTVSWNSGALTSGVHTFPA | |
| | VLQSSGLYSLSSVVTVPSSSLGTQTYICNVNHKPSNTKVDKK | |
| | VEPKSCDKTHTCPPCPAPELLGGPSVFLFPPKPKDTLMISRT | |
| | PEVTCVVVDVSHEDPEVKFNWYVDGVEVHNAKTKPREEQYNS | |
| | TYRVVSVLTVLHQDWLNGKEYKCKVSNKALPAPIEKTISKAK | |
| | GQPREPQVYTLPPCRDELTKNQVSLWCLVKGFYPSDIAVEWE | |
| | SNGQPENNYKTTPPVLDSDGSFFLYSKLTVDKSRWQQGNVFS | |
| | CSVMHEALHNRFTQKSLSLSPG | |
| Light | LC: | SEQ ID NO: 88 |
| chain B | IQMTQSPSSLSASVGDRVTITCRASQGIRNYLAWYQQKPGKA | |
| | PKLLIYAASTLQSGVPSRFSGSGSGTDFTLTISSLQPEDVAT | |
| | YYCQRYNRAPYTFGQGTKVEIKGGSGSSGSGGDIVLTQSPAS | |
| | LAVSLGQRATISCRASESVDSYGQSYMHWYQQKAGQPPKLLIY | |
| | LASNLESGVPARFSGSGSRTDFTLTIDPVQAEDAATYYC | |
| | QQNAEDSRTFGGGTKLEIKGGSGSSGSGGRTVAAPSVFIFPP | |
| | SDEQLKSGTASVVCLLNNFYPREAKVQWKVDNALQSGNSQES | |
| | VTEQDSKDSTYSLSSTLTLSKADYEKHKVYACEVTHQGLSSP | |
| | VTKSFNRGEC | |

Binding Protein 14_Nucleotide Sequences

| | | |
|---|---|---|
| Heavy | HC: | SEQ ID NO: 89 |
| chain A | caggtgcagctgcagcagagcggccccgagctggtgaagccc | |
| | ggcgccagcgtgaagatcagctgcaaggccagcggctacagc | |
| | ttcaccagctactggattcactggatcaagcagcgccccggc | |
| | cagggcctggagtggatcggcatgatcgaccccagcgacggc | |
| | gagacccgcctgaaccagcgcttccagggccgcgccaccctg | |
| | accgtggacgagagcaccagcaccgcctacatgcagctgcgc | |
| | agccccaccagcgaggacagcgccgtgtactactgcacccgc | |
| | ctgaaggagtacggcaactacgacagcttctacttcgacgtg | |
| | tggggcgccggcaccctggtgaccgtgagcagcgccagcacc | |
| | aagggccccagcgtgttccccctggcccctagcagcaagagc | |
| | acatctggcggcacagccgccctgggctgcctggtcaaggac | |
| | tacttccccgagcccgtgacagtgtcctggaactctggcgcc | |
| | ctgaccagcggagtgcataccttccctgccgtgctgcagtcc | |
| | agcggcctgtacagcctgagcagcgtggtcacagtgcccagc | |
| | agcagcctgggcacccagacctacatctgcaacgtgaaccac | |
| | aagcccagcaacaccaaggtggacaagaaggtggaacccaag | |
| | agctgcgacaagacccacacctgtcccccctgccctgcccct | |
| | gaactgctgggcggaccctccgtgttcctgttccccccaaag | |
| | cccaaggacaccctgatgatcagccggacccccgaagtgacc | |
| | tgcgtggtggtggacgtgtcccacgaggaccctgaagtgaag | |
| | ttcaattggtacgtggacggcgtggaagtgcataacgccaag | |
| | accaagcccagagaggaacagtacaacagcacctaccgggtg | |
| | gtgtccgtgctgaccgtgctgcaccaggactggctgaacggc | |
| | aaagagtacaagtgcaaggtgtccaacaaggccctgcctgcc | |
| | cccatcgagaaaaccatcagcaaggccaagggccagcctaga | |
| | gagccccaagtctgcaccctgccccccagcagagatgagctg | |
| | accaagaaccaggtgtccctgagctgcgccgtgaagggcttc | |
| | taccccagcgatatcgccgtggaatgggagagcaacggccag | |
| | cccgagaacaactacaagaccaccccccctgtgctggacagc | |
| | gacggctcattcttcctggtgtccaagctgacagtggacaag | |
| | agccggtggcagcagggcaacgtgttcagctgcagcgtgatg | |
| | cacgaggccctgcacaaccattacacccagaagtccctgagc | |
| | ctgagccccggc | |
| Light | LC: | SEQ ID NO: 90 |
| chain A | gacatccagatgacccagagccccgccagcctgagcgtgtcc | |
| | gtgggcgataccatcaccctgacctgccacgccagccagaac | |
| | atcgacgtgtggctgagctggttccagcagaagcccggcaac | |
| | atccccaagctgctgatctacaaggccagcaacctgcacacc | |
| | ggcgtgcccagcagattcagcggctctggcagcggcaccggc | |
| | tttaccctgaccatcagcagcctgcagcccgaggatatcgcc | |
| | acctactactgccagcaggcccacagctaccccttcaccttc | |
| | ggcggaggcaccaagctggaaatcaagggacagcccaaggct | |
| | gccccctcggtcaccctgttccccccaagctctgaggaactg | |
| | caggccaacaaggccaccctcgtgtgcctgatcagcgacttc | |
| | taccctggcgccgtgaccgtggcctggaaggccgatagctct | |
| | cccgtgaaggccggcgtggaaaccaccacccccagcaagcag | |
| | agcaacaacaaatacgccgccagcagctacctgagcctgacc | |
| | cccgagcagtggaagtcccaccggtcctacagctgccaggtc | |
| | acacacgagggcagcaccgtggaaaagaccgtggcccccacc | |
| | gagtgcagc | |
| Heavy | HC: | SEQ ID NO: 91 |
| chain B | gaggtgcagctgaaagagtccggccctggactggtggcccct | |
| | ggcggcagcctgagcatcacctgtaccgtgtccggcttcagc | |
| | ctgaccgacagcagcatcaactgggtccgacagccccctggc | |
| | aagggcctggaatggctgggcatgatctggggcgacggccgg | |
| | atcgactacgccgacgccctgaagtcccggctgagcatcagc | |
| | aaggacagcagcaagagccaggtgttcctggaaatgaccagc | |
| | ctgcggaccgacgacaccgccacctactactgcgccagggac | |
| | ggctacttcccctacgccatggatttctggggccagggcacc | |
| | agcgtgaccgtgtcctccgaagtgcagctggtggaaagcggc | |
| | ggaggcctggtgcagcccggcagaagcctgagactgagctgc | |
| | gccgccagcggcttcaccttcgacgactacgccatgcactgg | |
| | gtccgccaggctcccggaaagggactcgagtgggtgtccgcc | |
| | atcacctggaacagcggccacatcgattacgccgatagcgtg | |
| | gaaggccggttcaccatcagccgggacaacgccaagaacagc | |
| | ctgtacctgcagatgaacagcctgagagccgaggataccgcc | |
| | gtgtactactgtgccaaggtgtcctacctgagcaccgccagc | |
| | agcctggactactggggacagggaaccctggtcaccgtgtcc | |
| | agcgcttccaccaagggccccagcgtgttccctctggcccct | |
| | agcagcaagagcacatctggcggaacagccgccctgggctgc | |
| | ctggtcaaggactactttcccgagcccgtgaccgtgtcctgg | |
| | aactctggtgccctgacaagcggagtgcataccttccctgcc | |
| | gtgctgcagagcagcggcctgtactctctgagcagcgtggtc | |
| | accgtgccaagcagcagcctgggcacccagacctacatctgc | |
| | aacgtgaaccacaagccctccaacaccaaggtggacaagaag | |
| | gtggaacccaagagctgcgacaagacccacacctgtcctccc | |
| | tgtcctgcccctgaactgctgggcggaccctccgtgttcctg | |
| | ttccctccaaagcccaaggataccctgatgatcagccggacc | |
| | cctgaagtgacctgcgtggtggtggacgtgtcccacgaggat | |
| | cccgaagtgaagttcaattggtacgtggacggcgtggaagtg | |
| | cataacgccaagaccaagcccagagaggaacagtacaacagc | |
| | acctaccgggtggtgtccgtgctgacagtgctgcaccaggac | |
| | tggctgaacggcaaagagtacaagtgcaaggtgtccaacaag | |
| | gccctgccagcccctatcgagaaaaccatcagcaaggccaag | |
| | ggccagccccgcgagcctcaggtgtacacactgcctccatgc | |
| | cgggacgagctgaccaagaaccaggtgtccctgtggtgcctc | |
| | gtgaagggcttctacccctccgatatcgccgtggaatgggag | |
| | agcaacggccagcccgagaacaactacaagaccacccctccc | |
| | gtgctggacagcgacggctcattcttcctgtacagcaagctg | |
| | accgtggacaagtcccggtggcagcagggcaacgtgttcagc | |
| | tgctctgtgatgcacgaggccctgcacaaccggttcacccag | |
| | aagtccctgagcctgagccctggc | |
| Light | LC: | SEQ ID NO: 92 |
| chain B | gacatccagatgacccagagccccagcagcctgagcgccagc | |
| | gtgggcgacagagtgaccatcacctgtcgggccagccagggc | |
| | atccggaactacctggcctggtatcagcagaagcccggcaag | |
| | gcccccaagctgctgatctacgccgccagcacactgcagagc | |
| | ggcgtgcccagcagattcagcggcagcggctccggcaccgac | |
| | ttcaccctgaccatcagcagcctgcagcccgaggacgtggcc | |
| | acctactactgccagcggtacaacagagccccctacaccttc | |
| | ggccagggcaccaaggtggaaatcaagggcggctctggcagc | |
| | tccggctctggcggcgatatcgtgctgacccagtctcccgcc | |
| | agcctggccgtgtctctgggccagagagccaccatcagctgc | |
| | agagccagcgagagcgtggacagctacggccagagctacatg | |
| | cattggtatcagcagaaagccggccagcctcctaaactgctc | |
| | atctacctggccagcaacctggaatccggcgtgcccgccagg | |
| | ttttccggcagcggcagcagaaccgatttcacactgacaatc | |
| | gaccccgtgcaggccgaggatgccgccacatattactgtcag | |
| | cagaacgccgaggacagccggaccttcggcggaggcaccaag | |
| | ctcgagattaagggcggaagcggctccagcggcagtggcgga | |
| | cgtacggtggccgctccttccgtgttcatcttccctccctcc | |
| | gacgagcagctgaagtccggcaccgcctccgtggtgtgtctg | |
| | ctgaacaacttctaccctcgggaggccaaggtgcagtggaag | |
| | gtggacaacgccctgcagtccggcaactcccaggagtccgtc | |
| | accgagcaggactccaaggacagcacctactccctgtcctcc | |
| | accctgaccctgtccaaggccgactacgagaagcacaaggtg | |
| | tacgcctgtgaggtgacccaccagggcctgtccagccctgtg | |
| | accaagtccttcaaccggggcgagtgc | |

Binding Protein 15 Amino Acid Sequences

| | | |
|---|---|---|
| Heavy | HC: | SEQ ID NO: 85 |
| chain A | QVQLQQSGPELVKPGASVKISCKASGYSFTSYWIHWIKQRPG | |
| | QGLEWIGMIDPSDGETRLNQRFQGRATLTVDESTSTAYMQLR | |
| | SPTSEDSAVYYCTRLKEYGNYDSFYFDVWGAGTLVTVSSAST | |
| | KGPSVFPLAPSSKSTSGGTAALGCLVKDYFPEPVTVSWNSGA | |
| | LTSGVHTFPAVLQSSGLYSLSSVVTVPSSSLGTQTYICNVNH | |
| | KPSNTKVDKKVEPKSCDKTHTCPPCPAPELLGGPSVFLFPPK | |
| | PKDTLMISRTPEVTCVVVDVSHEDPEVKFNWYVDGVEVHNAK | |
| | TKPREEQYNSTYRVVSVLTVLHQDWLNGKEYKCKVSNKALPA | |
| | PIEKTISKAKGQPREPQVCTLPPSRDELTKNQVSLSCAVKGF | |
| | YPSDIAVEWESNGQPENNYKTTPPVLDSDGSFFLVSKLTVDK | |
| | SRWQQGNVFSCSVMHEALHNHYTQKSLSLSPG | |
| Light | LC: | SEQ ID NO: 86 |
| chain A | DIQMTQSPASLSVSVGDTITLTCHASQNIDVWLSWFQQKPGN | |
| | IPKLLIYKASNLHTGVPSRFSGSGSGTGFTLTISSLQPEDIA | |
| | TYYCQQAHSYPFTFGGGTKLEIKGQPKAAPSVTLFPPSSEEL | |
| | QANKATLVCLISDFYPGAVTVAWKADSSPVKAGVETTTPSKQ | |
| | SNNKYAASSYLSLTPEQWKSHRSYSCQVTHEGSTVEKTVAPT | |
| | ECS | |
| Heavy | HC: | SEQ ID NO: 93 |
| chain B | EVQLVESGGGLVQPGRSLRLSCAASGFTFDDYAMHWVRQAPG | |
| | KGLEWVSAITWNSGHIDYADSVEGRFTISRDNAKNSLYLQMN | |
| | SLRAEDTAVYYCAKVSYLSTASSLDYWGQGTLVTVSSEVQLK | |
| | ESGPGLVAPGGSLSITCTVSGFSLTDSSINWVRQPPGKGLEW | |
| | LGMIWGDGRIDYADALKSRLSISKDSSKSQVFLEMTSLRTDD | |
| | TATYYCARDGYFPYAMDFWGQGTSVTVSSASTKGPSVFPLAPS | |
| | SKSTSGGTAALGCLVKDYFPEPVTVSWNSGALTSGVHTFPAV | |
| | LQSSGLYSLSSVVTVPSSSLGTQTYICNVNHKPSNTKVDKKV | |
| | EPKSCDKTHTCPPCPAPELLGGPSVFLFPPKPKDTLMISRTP | |
| | EVTCVVVDVSHEDPEVKFNWYVDGVEVHNAKTKPREEQYNST | |
| | YRVVSVLTVLHQDWLNGKEYKCKVSNKALPAPIEKTISKAKG | |
| | QPREPQVYTLPPCRDELTKNQVSLWCLVKGFYPSDIAVEWES | |
| | NGQPENNYKTTPPVLDSDGSFFLYSKLTVDKSRWQQGNVFSC | |
| | SVMHEALHNRFTQKSLSLSPG | |
| Light | LC: | SEQ ID NO: 94 |
| chain B | DIVLTQSPASLAVSLGQRATISCRASESVDSYGQSYMHWYQQ | |
| | KAGQPPKLLIYLASNLESGVPARFSGSGSRTDFTLTIDPVQA | |
| | EDAATYYCQQNAEDSRTFGGGTKLEIKGGSGSSGSGGDIQMT | |
| | QSPSSLSASVGDRVTITCRASQGIRNYLAWYQQKPGKAPKLL | |
| | IYAASTLQSGVPSRFSGSGSGTDFTLTISSLQPEDVATYYC | |
| | QRYNRAPYTFGQGTKVEIKGGSGSSGSGGRTVAAPSVFIFPP | |
| | SDEQLKSGTASVVCLLNNFYPREAKVQWKVDNALQSGNSQES | |
| | VTEQDSKDSTYSLSSTLTLSKADYEKHKVYACEVTHQGLSSP | |
| | VTKSFNRGEC | |

Binding Protein 15 Nucleotide Sequences

| | | |
|---|---|---|
| Heavy | HC: | SEQ ID NO: 89 |
| chain A | caggtgcagctgcagcagagcggccccgagctggtgaagccc | |
| | ggcgccagcgtgaagatcagctgcaaggccagcggctacagc | |
| | ttcaccagctactggattcactggatcaagcagcgccccggc | |
| | cagggcctggagtggatcggcatgatcgaccccagcgacggc | |
| | gagacccgcctgaaccagcgcttccagggccgcgccaccctg | |
| | accgtggacgagagcaccagcaccgcctacatgcagctgcgc | |
| | agccccaccagcgaggacagcgccgtgtactactgcacccgc | |
| | ctgaaggagtacggcaactacgacagcttctacttcgacgtg | |
| | tggggcgccggcaccctggtgaccgtgagcagcgccagcacc | |
| | aagggccccagcgtgttccccctggcccctagcagcaagagc | |
| | acatctggcggcacagccgccctgggctgcctggtcaaggac | |
| | tacttccccgagcccgtgacagtgtcctggaactctggcgcc | |
| | ctgaccagcggagtgcataccttccctgccgtgctgcagtcc | |
| | agcggcctgtacagcctgagcagcgtggtcacagtgcccagc | |
| | agcagcctgggcacccagacctacatctgcaacgtgaaccac | |
| | aagcccagcaacaccaaggtggacaagaaggtggaacccaag | |
| | agctgcgacaagacccacacctgtcccccctgccctgcccct | |
| | gaactgctgggcggaccctccgtgttcctgttccccccaaag | |
| | cccaaggacaccctgatgatcagccggacccccgaagtgacc | |
| | tgcgtggtggtggacgtgtcccacgaggaccctgaagtgaag | |
| | ttcaattggtacgtggacggcgtggaagtgcataacgccaag | |
| | accaagcccagagaggaacagtacaacagcacctaccgggtg | |
| | gtgtccgtgctgaccgtgctgcaccaggactggctgaacggc | |
| | aaagagtacaagtgcaaggtgtccaacaaggccctgcctgcc | |
| | cccatcgagaaaaccatcagcaaggccaagggccagcctaga | |
| | gagccccaagtctgcaccctgccccccagcagagatgagctg | |
| | accaagaaccaggtgtccctgagctgcgccgtgaagggcttc | |
| | taccccagcgatatcgccgtggaatgggagagcaacggccag | |
| | cccgagaacaactacaagaccaccccccctgtgctggacagc | |
| | gacggctcattcttcctggtgtccaagctgacagtggacaag | |
| | agccggtggcagcagggcaacgtgttcagctgcagcgtgatg | |
| | cacgaggccctgcacaaccattacacccagaagtccctgagc | |
| | ctgagccccggc | |
| Light | LC: | SEQ ID NO: 90 |
| chain A | gacatccagatgacccagagccccgccagcctgagcgtgtcc | |
| | gtgggcgataccatcaccctgacctgccacgccagccagaac | |
| | atcgacgtgtggctgagctggttccagcagaagcccggcaac | |
| | atccccaagctgctgatctacaaggccagcaacctgcacacc | |
| | ggcgtgcccagcagattcagcggctctggcagcggcaccggc | |
| | tttaccctgaccatcagcagcctgcagcccgaggatatcgcc | |
| | acctactactgccagcaggcccacagctaccccttcaccttc | |
| | ggcggaggcaccaagctggaaatcaagggacagcccaaggct | |
| | gccccctcggtcaccctgttccccccaagctctgaggaactg | |
| | caggccaacaaggccaccctcgtgtgcctgatcagcgacttc | |
| | taccctggcgccgtgaccgtggcctggaaggccgatagctct | |
| | cccgtgaaggccggcgtggaaaccaccacccccagcaagcag | |
| | agcaacaacaaatacgccgccagcagctacctgagcctgacc | |
| | cccgagcagtggaagtcccaccggtcctacagctgccaggtc | |
| | acacacgagggcagcaccgtggaaaagaccgtggcccccacc | |
| | gagtgcagc | |
| Heavy | HC: | SEQ ID NO: 95 |
| chain B | gaggtgcagctggtggaaagcggcggaggactggtgcagccc | |
| | ggcagaagcctgagactgagctgcgccgccagcggcttcacc | |
| | ttcgacgactacgccatgcactgggtccgacaggcccctggc | |
| | aagggcctggaatgggtgtccgccatcacctggaacagcggc | |
| | cacatcgactacgccgacagcgtggaaggccggttcaccatc | |
| | agccgggacaacgccaagaacagcctgtacctgcagatgaac | |
| | agcctgcgggccgaggacaccgccgtgtactactgcgccaag | |
| | gtgtcctacctgagcaccgccagcagcctggactactggggc | |
| | cagggcaccctggtcaccgtgtcctccgaagtgcagctgaaa | |
| | gagtccggccctggcctggtggcccctggcggcagcctgagc | |
| | atcacctgtaccgtgtccggcttcagcctgaccgacagcagc | |
| | atcaactgggtccgccagcctcccggaaagggactcgagtgg | |
| | ctgggcatgatctggggcgacggccggatcgattacgccgat | |
| | gccctgaagtcccggctgagcatcagcaaggacagcagcaag | |
| | agccaggtgttcctggaaatgaccagcctgagaaccgacgac | |
| | accgccacctactactgtgcccgggacggctacttcccctac | |
| | gccatggatttctggggacagggaaccagcgtgaccgtgtcc | |
| | agcgcttccaccaagggccccagcgtgttccctctggcccct | |
| | agcagcaagagcacatctggcggaacagccgccctgggctgc | |
| | ctggtcaaggactactttcccgagcccgtgaccgtgtcctgg | |
| | aactctggtgccctgacaagcggagtgcataccttccctgcc | |
| | gtgctgcagagcagcggcctgtactctctgagcagcgtggtc | |
| | accgtgccaagcagcagcctgggcacccagacctacatctgc | |
| | aacgtgaaccacaagccctccaacaccaaggtggacaagaag | |
| | gtggaacccaagagctgcgacaagacccacacctgtcctccc | |
| | tgtcctgcccctgaactgctgggcggaccctccgtgttcctg | |
| | ttccctccaaagcccaaggataccctgatgatcagccggacc | |
| | cctgaagtgacctgcgtggtggtggacgtgtcccacgaggat | |
| | cccgaagtgaagttcaattggtacgtggacggcgtggaagtg | |
| | cataacgccaagaccaagcccagagaggaacagtacaacagc | |
| | acctaccgggtggtgtccgtgctgacagtgctgcaccaggac | |
| | tggctgaacggcaaagagtacaagtgcaaggtgtccaacaag | |
| | gccctgccagcccctatcgagaaaaccatcagcaaggccaag | |
| | ggccagccccgcgagcctcaggtgtacacactgcctccatgc | |
| | cgggacgagctgaccaagaaccaggtgtccctgtggtgcctc | |
| | gtgaagggcttctacccctccgatatcgccgtggaatgggag | |
| | agcaacggccagcccgagaacaactacaagaccacccctccc | |
| | gtgctggacagcgacggctcattcttcctgtacagcaagctg | |
| | accgtggacaagtcccggtggcagcagggcaacgtgttcagc | |
| | tgctctgtgatgcacgaggccctgcacaaccggttcacccag | |
| | aagtccctgagcctgagccctggc | |
| Light | LC: | SEQ ID NO: 96 |
| chain B | gacatcgtgctgacccagagccctgccagcctggccgtgtct | |
| | ctgggccagagagccaccatcagctgccgggccagcgagagc | |
| | gtggacagctacggccagagctacatgcactggtatcagcag | |
| | aaggccggccagccccccaagctgctgatctacctggccagc | |
| | aacctggaaagcggcgtgcccgccagattcagcggcagcggc | |
| | agcagaaccgacttcaccctgaccatcgaccccgtgcaggcc | |
| | gaggacgccgccacctactactgccagcagaacgccgaggac | |
| | agccggaccttcggcggaggcaccaagctggaaatcaagggc | |
| | ggctccggcagcagcggctctggcggcgatatccagatgacc | |
| | cagtcccccagcagcctgagcgccagcgtgggcgacagagtg | |
| | accatcacctgtagagccagccagggcatccggaactacctg | |
| | gcttggtatcagcagaaacccggaaaggcccctaaactgctc | |
| | atctacgccgccagcaccctgcagtccggcgtgccaagcaga | |
| | ttctccggctctggcagcggcaccgatttcacactgacaatc | |
| | agcagcctgcagcccgaggatgtggccacctattattgccag | |
| | agatacaacagagccccctacaccttcggccagggcaccaag | |
| | gtcgagattaagggcggaagcggcagctccggctccggcgga | |
| | cgtacggtggccgctccttccgtgttcatcttccctccctcc | |
| | gacgagcagctgaagtccggcaccgcctccgtggtgtgtctg | |
| | ctgaacaacttctaccctcgggaggccaaggtgcagtggaag | |
| | gtggacaacgccctgcagtccggcaactcccaggagtccgtc | |
| | accgagcaggactccaaggacagcacctactccctgtcctcc | |
| | accctgaccctgtccaaggccgactacgagaagcacaaggtg | |
| | tacgcctgtgaggtgacccaccagggcctgtccagccctgtg | |
| | accaagtccttcaaccggggcgagtgc | |

Binding Protein 16 Amino Acid Sequences

| | | |
|---|---|---|
| Heavy | HC: | SEQ ID NO: 73 |
| chain A | EVQLKESGPGLVAPGGSLSITCTVSGFSLTDSSINWVRQPPG | |
| | KGLEWLGMIWGDGRIDYADALKSRLSISKDSSKSQVFLEMTS | |
| | LRTDDTATYYCARDGYFPYAMDFWGQGTSVTVSSASTKGPSV | |
| | FPLAPSSKSTSGGTAALGCLVKDYFPEPVTVSWNSGALTSGV | |
| | HTFPAVLQSSGLYSLSSVVTVPSSSLGTQTYICNVNHKPSNT | |
| | KVDKKVEPKSCDKTHTCPPCPAPELLGGPSVFLFPPKPKDTL | |
| | MISRTPEVTCVVVDVSHEDPEVKFNWYVDGVEVHNAKTKPRE | |
| | EQYNSTYRVVSVLTVLHQDWLNGKEYKCKVSNKALPAPIEKT | |
| | ISKAKGQPREPQVCTLPPSRDELTKNQVSLSCAVKGFYPSDI | |
| | AVEWESNGQPENNYKTTPPVLDSDGSFFLVSKLTVDKSRWQQ | |
| | GNVFSCSVMHEALHNHYTQKSLSLSPG | |
| Light | LC: | SEQ ID NO: 74 |
| chain A | DIVLTQSPASLAVSLGQRATISCRASESVDSYGQSYMHWYQQ | |
| | KAGQPPKLLIYLASNLESGVPARFSGSGSRTDFTLTIDPVQA | |
| | EDAATYYCQQNAEDSRTFGGGTKLEIKGQPKAAPSVTLFPPS | |
| | SEELQANKATLVCLISDFYPGAVTVAWKADSSPVKAGVETTT | |
| | PSKQSNNKYAASSYLSLTPEQWKSHRSYSCQVTHEGSTVEKT | |
| | VAPTECS | |
| Heavy | HC: | SEQ ID NO: 68 |
| chain B | EVQLKESGPGLVAPGGSLSITCTVSGFSLTDSSINWVRQPPG | |
| | KGLEWLGMIWGDGRIDYADALKSRLSISKDSSKSQVFLEMTS | |
| | LRTDDTATYYCARDGYFPYAMDFWGQGTSVTVSSQVQLQQSG | |
| | PELVKPGASVKISCKASGYSFTSYWIHWIKQRPGQGLEWIG | |
| | MIDPSDGETRLNQRFQGRATLTVDESTSTAYMQLRSPTSEDS | |
| | AVYYCTRLKEYGNYDSFYFDVWGAGTLVTVSSASTKGPSVFP | |
| | LAPSSKSTSGGTAALGCLVKDYFPEPVTVSWNSGALTSGVHT | |
| | FPAVLQSSGLYSLSSVVTVPSSSLGTQTYICNVNHKPSNTKV | |
| | DKKVEPKSCDKTHTCPPCPAPELLGGPSVFLFPPKPKDTLMI | |
| | SRTPEVTCVVVDVSHEDPEVKFNWYVDGVEVHNAKTKPREEQ | |
| | YNSTYRVVSVLTVLHQDWLNGKEYKCKVSNKALPAPIEKTIS | |
| | KAKGQPREPQVYTLPPCRDELTKNQVSLWCLVKGFYPSDIAV | |
| | EWESNGQPENNYKTTPPVLDSDGSFFLYSKLTVDKSRWQQGN | |
| | VFSCSVMHEALHNRFTQKSLSLSPG | |
| Light | LC: | SEQ ID NO: 69 |
| chain B | DIQMTQSPASLSVSVGDTITLTCHASQNIDVWLSWFQQKPGN | |
| | IPKLLIYKASNLHTGVPSRFSGSGSGTGFTLTISSLQPEDIA | |
| | TYYCQQAHSYPFTFGGGTKLEIKGGSGSSGSGGDIVLTQSPA | |
| | SLAVSLGQRATISCRASESVDSYGQSYMHWYQQKAGQPPKLL | |
| | IYLASNLESGVPARFSGSGSRTDFTLTIDPVQAEDAATYYC | |
| | QQNAEDSRTFGGGTKLEIKGGSGSSGSGGRTVAAPSVFIFPP | |
| | SDEQLKSGTASVVCLLNNFYPREAKVQWKVDNALQSGNSQES | |
| | VTEQDSKDSTYSLSSTLTLSKADYEKHKVYACEVTHQGLSSP | |
| | VTKSFNRGEC | |

Binding Protein 16_Nucleotide Sequences

| | | |
|---|---|---|
| Heavy | HC: | SEQ ID NO: 77 |
| chain A | gaggtgcagctgaaggagagcggccccggcctggtggccccc | |
| | ggcggcagcctgagcatcacctgcaccgtgagcggcttcagc | |
| | ctgaccgacagcagcatcaactgggtgcgccagccccccggc | |
| | aagggcctggagtggctgggcatgatctggggcgacggccgc | |
| | atcgactacgccgacgccctgaagagccgcctgagcatcagc | |
| | aaggacagcagcaagagccaggtgttcctggagatgaccagc | |
| | ctgcgcaccgacgacaccgccacctactactgcgcccgcgac | |
| | ggctacttcccctacgccatggacttctggggccagggcacc | |
| | agcgtgaccgtgagcagcgccagcaccaagggccccagcgtg | |
| | ttccccctggcccctagcagcaagagcacatctggcggcaca | |
| | gccgccctgggctgcctggtcaaggactacttccccgagccc | |
| | gtgacagtgtcctggaactctggcgccctgaccagcggagtg | |
| | cataccttccctgccgtgctgcagtccagcggcctgtacagc | |
| | ctgagcagcgtggtcacagtgcccagcagcagcctgggcacc | |
| | cagacctacatctgcaacgtgaaccacaagcccagcaacacc | |
| | aaggtggacaagaaggtggaacccaagagctgcgacaagacc | |
| | cacacctgtcccccctgccctgcccctgaactgctgggcgga | |
| | ccctccgtgttcctgttccccccaaagcccaaggacaccctg | |
| | atgatcagccggacccccgaagtgacctgcgtggtggtggac | |
| | gtgtcccacgaggaccctgaagtgaagttcaattggtacgtg | |
| | gacggcgtggaagtgcataacgccaagaccaagcccagagag | |
| | gaacagtacaacagcacctaccgggtggtgtccgtgctgacc | |
| | gtgctgcaccaggactggctgaacggcaaagagtacaagtgc | |
| | aaggtgtccaacaaggccctgcctgcccccatcgagaaaacc | |
| | atcagcaaggccaagggccagcctagagagccccaagtctgc | |
| | accctgccccccagcagagatgagctgaccaagaaccaggtg | |
| | tccctgagctgcgccgtgaagggcttctaccccagcgatatc | |
| | gccgtggaatgggagagcaacggccagcccgagaacaactac | |
| | aagaccaccccccctgtgctggacagcgacggctcattcttc | |
| | ctggtgtccaagctgacagtggacaagagccggtggcagcag | |
| | ggcaacgtgttcagctgcagcgtgatgcacgaggccctgcac | |
| | aaccattacacccagaagtccctgagcctgagccccggc | |
| Light | LC: | SEQ ID NO: 78 |
| chain A | gacatcgtgctgacccagagccctgccagcctggccgtgtct | |
| | ctgggccagagagccaccatcagctgccgggccagcgagagc | |
| | gtggacagctacggccagagctacatgcactggtatcagcag | |
| | aaggccggccagccccccaagctgctgatctacctggccagc | |
| | aacctggaaagcggcgtgcccgccagattcagcggcagcggc | |
| | agcagaaccgacttcaccctgaccatcgaccccgtgcaggcc | |
| | gaggacgccgccacctactactgccagcagaacgccgaggac | |
| | agccggaccttcggcggaggcaccaagctggaaatcaaggga | |
| | cagcccaaggctgccccctcggtcaccctgttccccccaagc | |
| | agcgaggaactgcaggccaacaaggccaccctcgtgtgcctg | |
| | atcagcgacttctaccctggcgccgtgaccgtggcctggaag | |
| | gccgatagctctcccgtgaaggccggcgtggaaaccaccacc | |
| | cccagcaagcagagcaacaacaaatacgccgccagcagctac | |
| | ctgagcctgacccccgagcagtggaagtcccaccggtcctac | |
| | agctgccaggtcacacacgagggcagcaccgtggaaaagacc | |
| | gtggcccccaccgagtgcagc | |
| Heavy | HC: | SEQ ID NO: 97 |
| chain B | gaggtgcagctgaaagagtccggccctggactggtggcccct | |
| | ggcggcagcctgagcatcacctgtaccgtgtccggcttcagc | |
| | ctgaccgacagcagcatcaactgggtccgacagccccctggc | |
| | aagggcctggaatggctgggcatgatctggggcgacggccgg | |
| | atcgactacgccgacgccctgaagtcccggctgagcatcagc | |
| | aaggacagcagcaagagccaggtgttcctggaaatgaccagc | |
| | ctgcggaccgacgacaccgccacctactactgcgccagggac | |
| | ggctacttcccctacgccatggatttctggggccagggcacc | |
| | agcgtgaccgtgtccagtcaggtccagctgcagcagagcggc | |
| | cctgagctggtcaagcctggcgccagcgtgaagatcagctgc | |
| | aaggccagcggctacagcttcaccagctactggatccactgg | |
| | atcaagcagcggcctggccagggcctcgagtggatcggaatg | |
| | atcgaccccagcgacggcgagacacggctgaaccagagattc | |
| | cagggcagagccaccctgaccgtggacgagagcaccagcacc | |
| | gcctacatgcagctgcggagccccaccagcgaggacagcgcc | |
| | gtgtactactgcacccggctgaaagaatacggcaactacgac | |
| | agcttctacttcgacgtgtggggagccggcaccctggtcacc | |
| | gtgtctagcgcttccaccaagggccccagcgtgttccctctg | |
| | gcccctagcagcaagagcacatctggcggaacagccgccctg | |
| | ggctgcctggtcaaggactactttcccgagcccgtgaccgtg | |
| | tcctggaactctggtgccctgacaagcggagtgcataccttc | |
| | cctgccgtgctgcagagcagcggcctgtactctctgagcagc | |
| | gtggtcaccgtgccaagcagcagcctgggcacccagacctac | |
| | atctgcaacgtgaaccacaagccctccaacaccaaggtggac | |
| | aagaaggtggaacccaagagctgcgacaagacccacacctgt | |
| | cctccctgtcctgcccctgaactgctgggcggaccctccgtg | |
| | ttcctgttccctccaaagcccaaggataccctgatgatcagc | |
| | cggacccctgaagtgacctgcgtggtggtggacgtgtcccac | |
| | gaggatcccgaagtgaagttcaattggtacgtggacggcgtg | |
| | gaagtgcataacgccaagaccaagcccagagaggaacagtac | |
| | aacagcacctaccgggtggtgtccgtgctgacagtgctgcac | |
| | caggactggctgaacggcaaagagtacaagtgcaaggtgtcc | |
| | aacaaggccctgccagcccctatcgagaaaaccatcagcaag | |
| | gccaagggccagccccgcgagcctcaggtgtacacactgcct | |
| | ccatgccgggacgagctgaccaagaaccaggtgtccctgtgg | |
| | tgcctcgtgaagggcttctacccctccgatatcgccgtggaa | |
| | tgggagagcaacggccagcccgagaacaactacaagaccacc | |
| | cctcccgtgctggacagcgacggctcattcttcctgtacagc | |
| | aagctgaccgtggacaagtcccggtggcagcagggcaacgtg | |
| | ttcagctgctctgtgatgcacgaggccctgcacaaccggttc | |
| | acccagaagtccctgagcctgagccctggc | |
| Light | LC: | SEQ ID NO: 70 |
| chain B | gacatccagatgacccagagccccgccagcctgagcgtgtcc | |
| | gtgggcgataccatcaccctgacctgccacgccagccagaac | |
| | atcgacgtgtggctgagctggttccagcagaagcccggcaac | |
| | atccccaagctgctgatctacaaggccagcaacctgcacacc | |
| | ggcgtgcccagcagattcagcggctctggcagcggcaccggc | |
| | tttaccctgaccatcagcagcctgcagcccgaggatatcgcc | |
| | acctactactgccagcaggcccacagctaccccttcaccttc | |
| | ggcggaggcaccaagctggaaatcaagggcggcagcggcagc | |
| | tccggctctggcggcgatatcgtgctgacccagtctcccgcc | |
| | tccctggccgtgtctctgggccagagagccaccatcagctgc | |
| | cgggccagcgagagcgtggacagctacggccagagctacatg | |
| | cactggtatcagcagaaggccggacagccccctaaactgctc | |
| | atctacctggcctccaacctggaaagcggcgtgcccgccagg | |
| | ttttccggcagcggctccagaaccgacttcaccctgacaatc | |
| | gaccccgtgcaggccgaggacgccgccacatattactgtcag | |
| | cagaacgccgaggacagcagaacctttggcggcggaacaaag | |
| | ctcgagattaagggcggctccggctccagcggatctggcgga | |
| | cgtacggtggccgctccttccgtgttcatcttccctccctcc | |
| | gacgagcagctgaagtccggcaccgcctccgtggtgtgtctg | |
| | ctgaacaacttctaccctcgggaggccaaggtgcagtggaag | |
| | gtggacaacgccctgcagtccggcaactcccaggagtccgtc | |
| | accgagcaggactccaaggacagcacctactccctgtcctcc | |
| | accctgaccctgtccaaggccgactacgagaagcacaaggtg | |
| | tacgcctgtgaggtgacccaccagggcctgtccagccctgtg | |
| | accaagtccttcaaccggggcgagtgc | |

Binding Protein 17 Amino Acid Sequences

| | | |
|---|---|---|
| Heavy | HC: | SEQ ID NO: 85 |
| chain A | QVQLQQSGPELVKPGASVKISCKASGYSFTSYWIHWIKQRPG | |
| | QGLEWIGMIDPSDGETRLNQRFQGRATLTVDESTSTAYMQLR | |
| | SPTSEDSAVYYCTRLKEYGNYDSFYFDVWGAGTLVTVSSAST | |
| | KGPSVFPLAPSSKSTSGGTAALGCLVKDYFPEPVTVSWNSGA | |
| | LTSGVHTFPAVLQSSGLYSLSSVVTVPSSSLGTQTYICNVNH | |
| | KPSNTKVDKKVEPKSCDKTHTCPPCPAPELLGGPSVFLFPPK | |
| | PKDTLMISRTPEVTCVVVDVSHEDPEVKFNWYVDGVEVHNAK | |
| | TKPREEQYNSTYRVVSVLTVLHQDWLNGKEYKCKVSNKALPA | |
| | PIEKTISKAKGQPREPQVCTLPPSRDELTKNQVSLSCAVKGF | |
| | YPSDIAVEWESNGQPENNYKTTPPVLDSDGSFFLVSKLTVDK | |
| | SRWQQGNVFSCSVMHEALHNHYTQKSLSLSPG | |
| Light | LC: | SEQ ID NO: 86 |
| chain A | DIQMTQSPASLSVSVGDTITLTCHASQNIDVWLSWFQQKPGN | |
| | IPKLLIYKASNLHTGVPSRFSGSGSGTGFTLTISSLQPEDIA | |
| | TYYCQQAHSYPFTFGGGTKLEIKGQPKAAPSVTLFPPSSEEL | |
| | QANKATLVCLISDFYPGAVTVAWKADSSPVKAGVETTTPSKQ | |
| | SNNKYAASSYLSLTPEQWKSHRSYSCQVTHEGSTVEKTVAPT | |
| | ECS | |
| Heavy | HC: | SEQ ID NO: 68 |
| chain B | EVQLKESGPGLVAPGGSLSITCTVSGFSLTDSSINWVRQPP | |
| | GKGLEWLGMIWGDGRIDYADALKSRLSISKDSSKSQVFLEMT | |
| | SLRTDDTATYYCARDGYFPYAMDFWGQGTSVTVSSQVQLQQS | |
| | GPELVKPGASVKISCKASGYSFTSYWIHWIKQRPGQGLEWIG | |
| | MIDPSDGETRLNQRFQGRATLTVDESTSTAYMQLRSPTSEDS | |
| | AVYYCTRLKEYGNYDSFYFDVWGAGTLVTVSSASTKGPSVFP | |
| | LAPSSKSTSGGTAALGCLVKDYFPEPVTVSWNSGALTSGVHT | |
| | FPAVLQSSGLYSLSSVVTVPSSSLGTQTYICNVNHKPSNTKV | |
| | DKKVEPKSCDKTHTCPPCPAPELLGGPSVFLFPPKPKDTLMI | |
| | SRTPEVTCVVVDVSHEDPEVKFNWYVDGVEVHNAKTKPREEQ | |
| | YNSTYRVVSVLTVLHQDWLNGKEYKCKVSNKALPAPIEKTIS | |
| | KAKGQPREPQVYTLPPCRDELTKNQVSLWCLVKGFYPSDIAV | |
| | EWESNGQPENNYKTTPPVLDSDGSFFLYSKLTVDKSRWQQGN | |
| | VFSCSVMHEALHNRFTQKSLSLSPG | |
| Light | LC: | SEQ ID NO: 69 |
| chain B | DIQMTQSPASLSVSVGDTITLTCHASQNIDVWLSWFQQKPGN | |
| | IPKLLIYKASNLHTGVPSRFSGSGSGTGFTLTISSLQPEDIA | |
| | TYYCQQAHSYPFTFGGGTKLEIKGGSGSSGSGGDIVLTQSPA | |
| | SLAVSLGQRATISCRASESVDSYGQSYMHWYQQKAGQPPKLLIY | |
| | LASNLESGVPARFSGSGSRTDFTLTIDPVQAEDAATYYC | |
| | QQNAEDSRTFGGGTKLEIKGGSGSSGSGGRTVAAPSVFIFPP | |
| | SDEQLKSGTASVVCLLNNFYPREAKVQWKVDNALQSGNSQES | |
| | VTEQDSKDSTYSLSSTLTLSKADYEKHKVYACEVTHQGLSSP | |
| | VTKSFNRGEC | |

Binding Protein 17_Nucleotide Sequences

| | | |
|---|---|---|
| Heavy | HC: | SEQ ID NO: 89 |
| chain A | caggtgcagctgcagcagagcggccccgagctggtgaagccc | |
| | ggcgccagcgtgaagatcagctgcaaggccagcggctacagc | |
| | ttcaccagctactggattcactggatcaagcagcgccccggc | |
| | cagggcctggagtggatcggcatgatcgaccccagcgacggc | |
| | gagacccgcctgaaccagcgcttccagggccgcgccaccctg | |
| | accgtggacgagagcaccagcaccgcctacatgcagctgcgc | |
| | agccccaccagcgaggacagcgccgtgtactactgcacccgc | |
| | ctgaaggagtacggcaactacgacagcttctacttcgacgtg | |
| | tggggcgccggcaccctggtgaccgtgagcagcgccagcacc | |
| | aagggccccagcgtgttccccctggcccctagcagcaagagc | |
| | acatctggcggcacagccgccctgggctgcctggtcaaggac | |
| | tacttccccgagcccgtgacagtgtcctggaactctggcgcc | |
| | ctgaccagcggagtgcataccttccctgccgtgctgcagtcc | |
| | agcggcctgtacagcctgagcagcgtggtcacagtgcccagc | |
| | agcagcctgggcacccagacctacatctgcaacgtgaaccac | |
| | aagcccagcaacaccaaggtggacaagaaggtggaacccaag | |
| | agctgcgacaagacccacacctgtcccccctgccctgcccct | |
| | gaactgctgggcggaccctccgtgttcctgttccccccaaag | |
| | cccaaggacaccctgatgatcagccggacccccgaagtgacc | |
| | tgcgtggtggtggacgtgtcccacgaggaccctgaagtgaag | |
| | ttcaattggtacgtggacggcgtggaagtgcataacgccaag | |
| | accaagcccagagaggaacagtacaacagcacctaccgggtg | |
| | gtgtccgtgctgaccgtgctgcaccaggactggctgaacggc | |
| | aaagagtacaagtgcaaggtgtccaacaaggccctgcctgcc | |
| | cccatcgagaaaaccatcagcaaggccaagggccagcctaga | |
| | gagccccaagtctgcaccctgccccccagcagagatgagctg | |
| | accaagaaccaggtgtccctgagctgcgccgtgaagggcttc | |
| | taccccagcgatatcgccgtggaatgggagagcaacggccag | |
| | cccgagaacaactacaagaccaccccccctgtgctggacagc | |
| | gacggctcattcttcctggtgtccaagctgacagtggacaag | |
| | agccggtggcagcagggcaacgtgttcagctgcagcgtgatg | |
| | cacgaggccctgcacaaccattacacccagaagtccctgagc | |
| | ctgagccccggc | |
| Light | LC: | SEQ ID NO: 90 |
| chain A | Gacatccagatgacccagagccccgccagcctgagcgtgtcc | |
| | gtgggcgataccatcaccctgacctgccacgccagccagaac | |
| | atcgacgtgtggctgagctggttccagcagaagcccggcaac | |
| | atccccaagctgctgatctacaaggccagcaacctgcacacc | |
| | ggcgtgcccagcagattcagcggctctggcagcggcaccggc | |
| | tttaccctgaccatcagcagcctgcagcccgaggatatcgcc | |
| | acctactactgccagcaggcccacagctaccccttcaccttc | |
| | ggcggaggcaccaagctggaaatcaagggacagcccaaggct | |
| | gccccctcggtcaccctgttccccccaagctctgaggaactg | |
| | caggccaacaaggccaccctcgtgtgcctgatcagcgacttc | |
| | taccctggcgccgtgaccgtggcctggaaggccgatagctct | |
| | cccgtgaaggccggcgtggaaaccaccacccccagcaagcag | |
| | agcaacaacaaatacgccgccagcagctacctgagcctgacc | |
| | cccgagcagtggaagtcccaccggtcctacagctgccaggtc | |
| | acacacgagggcagcaccgtggaaaagaccgtggcccccacc | |
| | gagtgcagc | |
| Heavy | HC: | SEQ ID NO: 97 |
| chain B | gaggtgcagctgaaagagtccggccctggactggtggcccct | |
| | ggcggcagcctgagcatcacctgtaccgtgtccggcttcagc | |
| | ctgaccgacagcagcatcaactgggtccgacagccccctggc | |
| | aagggcctggaatggctgggcatgatctggggcgacggccgg | |
| | atcgactacgccgacgccctgaagtcccggctgagcatcagc | |
| | aaggacagcagcaagagccaggtgttcctggaaatgaccagc | |
| | ctgcggaccgacgacaccgccacctactactgcgccagggac | |
| | ggctacttcccctacgccatggatttctggggccagggcacc | |
| | agcgtgaccgtgtccagtcaggtccagctgcagcagagcggc | |
| | cctgagctggtcaagcctggcgccagcgtgaagatcagctgc | |
| | aaggccagcggctacagcttcaccagctactggatccactgg | |
| | atcaagcagcggcctggccagggcctcgagtggatcggaatg | |
| | atcgaccccagcgacggcgagacacggctgaaccagagattc | |
| | cagggcagagccaccctgaccgtggacgagagcaccagcacc | |
| | gcctacatgcagctgcggagccccaccagcgaggacagcgcc | |
| | gtgtactactgcacccggctgaaagaatacggcaactacgac | |
| | agcttctacttcgacgtgtggggagccggcaccctggtcacc | |
| | gtgtctagcgcttccaccaagggccccagcgtgttccctctg | |
| | gcccctagcagcaagagcacatctggcggaacagccgccctg | |
| | ggctgcctggtcaaggactactttcccgagcccgtgaccgtg | |
| | tcctggaactctggtgccctgacaagcggagtgcataccttc | |
| | cctgccgtgctgcagagcagcggcctgtactctctgagcagc | |
| | gtggtcaccgtgccaagcagcagcctgggcacccagacctac | |
| | atctgcaacgtgaaccacaagccctccaacaccaaggtggac | |
| | aagaaggtggaacccaagagctgcgacaagacccacacctgt | |
| | cctccctgtcctgcccctgaactgctgggcggaccctccgtg | |
| | ttcctgttccctccaaagcccaaggataccctgatgatcagc | |
| | cggacccctgaagtgacctgcgtggtggtggacgtgtcccac | |
| | gaggatcccgaagtgaagttcaattggtacgtggacggcgtg | |
| | gaagtgcataacgccaagaccaagcccagagaggaacagtac | |
| | aacagcacctaccgggtggtgtccgtgctgacagtgctgcac | |
| | caggactggctgaacggcaaagagtacaagtgcaaggtgtcc | |
| | aacaaggccctgccagcccctatcgagaaaaccatcagcaag | |
| | gccaagggccagccccgcgagcctcaggtgtacacactgcct | |
| | ccatgccgggacgagctgaccaagaaccaggtgtccctgtgg | |
| | tgcctcgtgaagggcttctacccctccgatatcgccgtggaa | |
| | tgggagagcaacggccagcccgagaacaactacaagaccacc | |
| | cctcccgtgctggacagcgacggctcattcttcctgtacagc | |
| | aagctgaccgtggacaagtcccggtggcagcagggcaacgtg | |
| | ttcagctgctctgtgatgcacgaggccctgcacaaccggttc | |
| | acccagaagtccctgagcctgagccctggc | |
| Light | LC: | SEQ ID NO: 70 |
| chain B | gacatccagatgacccagagccccgccagcctgagcgtgtcc | |
| | gtgggcgataccatcaccctgacctgccacgccagccagaac | |
| | atcgacgtgtggctgagctggttccagcagaagcccggcaac | |
| | atccccaagctgctgatctacaaggccagcaacctgcacacc | |
| | ggcgtgcccagcagattcagcggctctggcagcggcaccggc | |
| | tttaccctgaccatcagcagcctgcagcccgaggatatcgcc | |
| | acctactactgccagcaggcccacagctaccccttcaccttc | |
| | ggcggaggcaccaagctggaaatcaagggcggcagcggcagc | |
| | tccggctctggcggcgatatcgtgctgacccagtctcccgcc | |
| | tccctggccgtgtctctgggccagagagccaccatcagctgc | |
| | cgggccagcgagagcgtggacagctacggccagagctacatg | |
| | cactggtatcagcagaaggccggacagccccctaaactgctc | |
| | atctacctggcctccaacctggaaagcggcgtgcccgccagg | |
| | ttttccggcagcggctccagaaccgacttcaccctgacaatc | |
| | gaccccgtgcaggccgaggacgccgccacatattactgtcag | |
| | cagaacgccgaggacagcagaacctttggcggcggaacaaag | |
| | ctcgagattaagggcggctccggctccagcggatctggcgga | |
| | cgtacggtggccgctccttccgtgttcatcttccctccctcc | |
| | gacgagcagctgaagtccggcaccgcctccgtggtgtgtctg | |
| | ctgaacaacttctaccctcgggaggccaaggtgcagtggaag | |
| | gtggacaacgccctgcagtccggcaactcccaggagtccgtc | |
| | accgagcaggactccaaggacagcacctactccctgtcctcc | |
| | accctgaccctgtccaaggccgactacgagaagcacaaggtg | |
| | tacgcctgtgaggtgacccaccagggcctgtccagccctgtg | |
| | accaagtccttcaaccggggcgagtgc | |

Binding Protein 18 Amino Acid Sequences

| | | |
|---|---|---|
| Heavy | HC: | SEQ ID NO: 73 |
| chain A | EVQLKESGPGLVAPGGSLSITCTVS [symbol] WVRQPPG | |
| | KGLEWLG [symbol] YADALKSRLSISKDSSKSQVFLEMTS | |
| | LRTDDTATYYCAR [symbol] WGQGTSVTVSSASTKGPSV | |
| | FPLAPSSKSTSGGTAALGCLVKDYFPEPVTVSWNSGALTSGV | |
| | HTFPAVLQSSGLYSLSSVVTVPSSSLGTQTYICNVNHKPSNT | |
| | KVDKKVEPKSCDKTHTCPPCPAPELLGGPSVFLFPPKPKDTL | |
| | MISRTPEVTCVVVDVSHEDPEVKFNWYVDGVEVHNAKTKPRE | |
| | EQYNSTYRVVSVLTVLHQDWLNGKEYKCKVSNKALPAPIEKT | |
| | ISKAKGQPREPQVCTLPPSRDELTKNQVSLSCAVKGFYPSDI | |
| | AVEWESNGQPENNYKTTPPVLDSDGSFFLVSKLTVDKSRWQQ | |
| | GNVFSCSVMHEALHNHYTQKSLSLSPG | |
| Light | LC: | SEQ ID NO: 74 |
| chain A | DIVLTQSPASLAVSLGQRATISC [symbol] WYQQ | |
| | KAGQPPKLLIY [symbol] GVPARFSGSGSRTDFTLTIDPVQA | |
| | EDAATYYC [symbol] FGGGTKLEIKGQPKAAPSVTLFPPS | |
| | SEELQANKATLVCLISDFYPGAVTVAWKADSSPVKAGVETTT | |
| | PSKQSNNKYAASSYLSLTPEQWKSHRSYSCQVTHEGSTVEKT | |
| | VAPTECS | |
| Heavy | HC: | SEQ ID NO: 62 |
| chain B | QVQLQQSGPELVKPGASVKISCKASGYSFTSYWIHWIKQRPG | |
| | QGLEWIGMIDPSDGETRLNQRFQGRATLTVDESTSTAYMQLR | |
| | SPTSEDSAVYYCTRLKEYGNYDSFYFDVWGAGTLVTVSSEVQ | |
| | LKESGPGLVAPGGSLSITCTVSGFSLTDSSINNWVRQPPGKGL | |
| | EWLGMIWGDGRIDYADALKSRLSISKDSSKSQVFLEMTSLRT | |
| | DDTATYYCARDGYFPYAMDFWGQGTSVTVSSASTKGPSVFPL | |
| | APSSKSTSGGTAALGCLVKDYFPEPVTVSWNSGALTSGVHTF | |
| | PAVLQSSGLYSLSSVVTVPSSSLGTQTYICNVNHKPSNTKVD | |
| | KKVEPKSCDKTHTCPPCPAPELLGGPSVFLFPPKPKDTLMIS | |
| | RTPEVTCVVVDVSHEDPEVKFNWYVDGVEVHNAKTKPREEQY | |
| | NSTYRVVSVLTVLHQDWLNGKEYKCKVSNKALPAPIEKTISK | |
| | AKGQPREPQVYTLPPCRDELTKNQVSLWCLVKGFYPSDIAVE | |
| | WESNGQPENNYKTTPPVLDSDGSFFLYSKLTVDKSRWQQGNV | |
| | FSCSVMHEALHNRFTQKSLSLSPG | |
| Light | LC: | SEQ ID NO: 63 |
| chain B | DIVLTQSPASLAVSLGQRATISCRASESVDSYGQSYMHWYQQ | |
| | KAGQPPKLLIYLASNLESGVPARFSGSGSRTDFTLTIDPVQA | |
| | EDAATYYCQQNAEDSRTFGGGTKLEIKGGSGSSGSGGDIQMT | |
| | QSPASLSVSVGDTITLTCHASQNIDVWLSWFQQKPGNIPKLL | |
| | IYKASNLHTGVPSRFSGSGSGTGFTLTISSLQPEDIATYYC | |
| | QQAHSYPFTFGGGTKLEIKGGSGSSGSGGRTVAAPSVFIFPP | |
| | SDEQLKSGTASVVCLLNNFYPREAKVQWKVDNALQSGNSQES | |
| | VTEQDSKDSTYSLSSTLTLSKADYEKHKVYACEVTHQGLSSP | |
| | VTKSFNRGEC | |

Binding Protein 18 Nucleotide Sequences

| | | |
|---|---|---|
| Heavy | HC: | SEQ ID NO: 77 |
| chain A | gaggtgcagctgaaggagagcggccccggcctggtggccccc | |
| | ggcggcagcctgagcatcacctgcaccgtgagcggcttcagc | |
| | ctgaccgacagcagcatcaactgggtgcgccagccccccggc | |
| | aagggcctggagtggctgggcatgatctggggcgacggccgc | |
| | atcgactacgccgacgccctgaagagccgcctgagcatcagc | |
| | aaggacagcagcaagagccaggtgttcctggagatgaccagc | |
| | ctgcgcaccgacgacaccgccacctactactgcgcccgcgac | |
| | ggctacttcccctacgccatggacttctggggccagggcacc | |
| | agcgtgaccgtgagcagcgccagcaccaagggccccagcgtg | |
| | ttccccctggcccctagcagcaagagcacatctggcggcaca | |
| | gccgccctgggctgcctggtcaaggactacttccccgagccc | |
| | gtgacagtgtcctggaactctggcgccctgaccagcggagtg | |
| | cataccttccctgccgtgctgcagtccagcggcctgtacagc | |
| | ctgagcagcgtggtcacagtgcccagcagcagcctgggcacc | |
| | cagacctacatctgcaacgtgaaccacaagcccagcaacacc | |
| | aaggtggacaagaaggtggaacccaagagctgcgacaagacc | |
| | cacacctgtcccccctgccctgcccctgaactgctgggcgga | |
| | ccctccgtgttcctgttccccccaaagcccaaggacaccctg | |
| | atgatcagccggacccccgaagtgacctgcgtggtggtggac | |
| | gtgtcccacgaggaccctgaagtgaagttcaattggtacgtg | |
| | gacggcgtggaagtgcataacgccaagaccaagcccagagag | |
| | gaacagtacaacagcacctaccgggtggtgtccgtgctgacc | |
| | gtgctgcaccaggactggctgaacggcaaagagtacaagtgc | |
| | aaggtgtccaacaaggccctgcctgcccccatcgagaaaacc | |
| | atcagcaaggccaagggccagcctagagagccccaagtctgc | |
| | accctgccccccagcagagatgagctgaccaagaaccaggtg | |
| | tccctgagctgcgccgtgaagggcttctaccccagcgatatc | |
| | gccgtggaatgggagagcaacggccagcccgagaacaactac | |
| | aagaccaccccccctgtgctggacagcgacggctcattcttc | |
| | ctggtgtccaagctgacagtggacaagagccggtggcagcag | |
| | ggcaacgtgttcagctgcagcgtgatgcacgaggccctgcac | |
| | aaccattacacccagaagtccctgagcctgagccccggc | |
| Light | LC: | SEQ ID NO: 78 |
| chain A | gacatcgtgctgacccagagccctgccagcctggccgtgtct | |
| | ctgggccagagagccaccatcagctgccgggccagcgagagc | |
| | gtggacagctacggccagagctacatgcactggtatcagcag | |
| | aaggccggccagccccccaagctgctgatctacctggccagc | |
| | aacctggaaagcggcgtgcccgccagattcagcggcagcggc | |
| | agcagaaccgacttcaccctgaccatcgaccccgtgcaggcc | |
| | gaggacgccgccacctactactgccagcagaacgccgaggac | |
| | agccggaccttcggcggaggcaccaagctggaaatcaaggga | |
| | cagcccaaggctgccccctcggtcaccctgttccccccaagc | |
| | agcgaggaactgcaggccaacaaggccaccctcgtgtgcctg | |
| | atcagcgacttctaccctggcgccgtgaccgtggcctggaag | |
| | gccgatagctctcccgtgaaggccggcgtggaaaccaccacc | |
| | cccagcaagcagagcaacaacaaatacgccgccagcagctac | |
| | ctgagcctgacccccgagcagtggaagtcccaccggtcctac | |
| | agctgccaggtcacacacgagggcagcaccgtggaaaagacc | |
| | gtggcccccaccgagtgcagc | |
| Heavy | HC: | SEQ ID NO: 66 |
| chain B | caggtgcagctgcagcagagcggccctgagctggtcaagcct | |
| | ggcgccagcgtgaagatcagctgcaaggccagcggctacagc | |
| | ttcaccagctactggatccactggatcaagcagcggcctggc | |
| | cagggcctggaatggatcggcatgatcgaccccagcgacggc | |
| | gagacacggctgaaccagagattccagggcagagccaccctg | |
| | accgtggacgagagcaccagcaccgcctacatgcagctgcgg | |
| | agccccaccagcgaggacagcgccgtgtactactgcacccgg | |
| | ctgaaagagtacggcaactacgacagatctacttcgacgtgt | |
| | ggggagccggcaccctggtcaccgtgtccagcgaagtgcagc | |
| | tgaaagaaagcggccctggcctggtggcccctggcggcagcc | |
| | tgagcatcacctgtaccgtgtccggcttcagcctgaccgaca | |
| | gcagcatcaactgggtccgacagccccctggcaagggcctcg | |
| | agtggctgggaatgatctggggcgacggccggatcgactacg | |
| | ccgacgccctgaagtcccggctgagcatcagcaaggacagca | |
| | gcaagagccaggtgttcctggaaatgaccagcctgcggaccg | |
| | acgacaccgccacctactactgcgccagggacggctacttcc | |
| | cctacgccatggatttctggggccagggcaccagcgtgaccg | |
| | tgtcctctgcttccaccaagggccccagcgtgttccctctgg | |
| | cccctagcagcaagagcacatctggcggaacagccgccctgg | |
| | gctgcctggtcaaggactactttcccgagcccgtgaccgtgt | |
| | cctggaactctggtgccctgacaagcggagtgcataccttcc | |
| | ctgccgtgctgcagagcagcggcctgtactctctgagcagcg | |
| | tggtcaccgtgccaagcagcagcctgggcacccagacctaca | |
| | tctgcaacgtgaaccacaagccctccaacaccaaggtggaca | |
| | agaaggtggaacccaagagctgcgacaagacccacacctgtc | |
| | ctccctgtcctgcccctgaactgctgggcggaccctccgtgt | |
| | tcctgttccctccaaagcccaaggataccctgatgatcagcc | |
| | ggacccctgaagtgacctgcgtggtggtggacgtgtcccacg | |
| | aggatcccgaagtgaagttcaattggtacgtggacggcgtgg | |
| | aagtgcataacgccaagaccaagcccagagaggaacagtaca | |
| | acagcacctaccgggtggtgtccgtgctgacagtgctgcacc | |
| | aggactggctgaacggcaaagagtacaagtgcaaggtgtcca | |
| | acaaggccctgccagcccctatcgagaaaaccatcagcaagg | |
| | ccaagggccagccccgcgagcctcaggtgtacacactgcctc | |
| | catgccgggacgagctgaccaagaaccaggtgtccctgtggt | |
| | gcctcgtgaagggcttctacccctccgatatcgccgtggaat | |
| | gggagagcaacggccagcccgagaacaactacaagaccaccc | |
| | ctcccgtgctggacagcgacggctcattcttcctgtacagca | |
| | agctgaccgtggacaagtcccggtggcagcagggcaacgtgt | |
| | tcagctgctctgtgatgcacgaggccctgcacaaccggttca | |
| | cccagaagtccctgagcctgagccctggc | |
| Light | LC: | SEQ ID NO: 67 |
| chain B | Gacatcgtgctgacccagagccctgccagcctggccgtgtct | |
| | ctgggccagagagccaccatcagctgccgggccagcgagagc | |
| | gtggacagctacggccagagctacatgcactggtatcagcag | |
| | aaggccggccagccccccaagctgctgatctacctggccagc | |
| | aacctggaaagcggcgtgcccgccagattcagcggcagcggc | |
| | agcagaaccgacttcaccctgaccatcgaccccgtgcaggcc | |
| | gaggacgccgccacctactactgccagcagaacgccgaggac | |
| | agccggaccttcggcggaggcaccaagctggaaatcaagggc | |
| | ggctccggcagcagcggctctggcggcgatatccagatgacc | |
| | cagtcccccgcctccctgagcgtgtccgtgggcgacaccatc | |
| | accctgacatgccacgccagccagaacatcgacgtgtggctg | |
| | agctggttccagcagaagcctggcaacatccctaagctgctc | |
| | atctataaggcctccaacctgcacaccggcgtgcccagcagg | |
| | ttttccggctctggcagcggcaccggctttaccctgacaatc | |
| | agcagcctgcagcccgaggatatcgccacatattactgtcag | |
| | caggcccacagctaccccttcacctttggcggcggaacaaag | |
| | ctcgagattaagggcggcagcggaagctccggctccggcgga | |
| | cgtacggtggccgctccttccgtgttcatcttccctccctcc | |
| | gacgagcagctgaagtccggcaccgcctccgtggtgtgtctg | |
| | ctgaacaacttctaccctcgggaggccaaggtgcagtggaag | |
| | gtggacaacgccctgcagtccggcaactcccaggagtccgtc | |
| | accgagcaggactccaaggacagcacctactccctgtcctcc | |
| | accctgaccctgtccaaggccgactacgagaagcacaaggtg | |
| | tacgcctgtgaggtgacccaccagggcctgtccagccctgtg | |
| | accaagtccttcaaccggggcgagtgc | |

Binding Protein 19 Amino Acid Sequences

| | | |
|---|---|---|
| Heavy | HC: | SEQ ID NO: 85 |
| chain A | QVQLQQSGPELVKPGASVKISCKASGYSFTSYWIHWIKQRPG | |
| | QGLEWIGMIDPSDGETRLNQRFQGRATLTVDESTSTAYMQLR | |
| | SPTSEDSAVYYCTRLKEYGNYDSFYFDVWGAGTLVTVSSAST | |
| | KGPSVFPLAPSSKSTSGGTAALGCLVKDYFPEPVTVSWNSGA | |
| | LTSGVHTFPAVLQSSGLYSLSSVVTVPSSSLGTQTYICNVNH | |
| | KPSNTKVDKKVEPKSCDKTHTCPPCPAPELLGGPSVFLFPPK | |
| | PKDTLMISRTPEVTCVVVDVSHEDPEVKFNWYVDGVEVHNAK | |
| | TKPREEQYNSTYRVVSVLTVLHQDWLNGKEYKCKVSNKALPA | |
| | PIEKTISKAKGQPREPQVCTLPPSRDELTKNQVSLSCAVKGF | |
| | YPSDIAVEWESNGQPENNYKTTPPVLDSDGSFFLVSKLTVDK | |
| | SRWQQGNVFSCSVMHEALHNHYTQKSLSLSPG | |
| Light | LC: | SEQ ID NO: 86 |
| chain A | DIQMTQSPASLSVSVGDTITLTCHASQNIDVWLSWFQQKPGN | |
| | IPKLLIYKASNLHTGVPSRFSGSGSGTGFTLTISSLQPEDIA | |
| | TYYCQQAHSYPFTFGGGTKLEIKGQPKAAPSVTLFPPSSEEL | |
| | QANKATLVCLISDFYPGAVTVAWKADSSPVKAGVETTTPSKQ | |
| | SNNKYAASSYLSLTPEQWKSHRSYSCQVTHEGSTVEKTVAPT | |
| | ECS | |
| Heavy | HC: | SEQ ID NO: 62 |
| chain B | QVQLQQSGPELVKPGASVKISCKASGYSFTSYWIHWIKQRPG | |
| | QGLEWIGMIDPSDGETRLNQRFQGRATLTVDESTSTAYMQLR | |
| | SPTSEDSAVYYCTRLKEYGNYDSFYFDVWGAGTLVTVSSEVQ | |
| | LKESGPGLVAPGGSLSITCTVSGFSLTDSSINWVRQPPGKGL | |
| | EWLGMIWGDGRIDYADALKSRLSISKDSSKSQVFLEMTSLRT | |
| | DDTATYYCARDGYFPYAMDFWGQGTSVTVSSASTKGPSVFPL | |
| | APSSKSTSGGTAALGCLVKDYFPEPVTVSWNSGALTSGVHTF | |
| | PAVLQSSGLYSLSSVVTVPSSSLGTQTYICNVNHKPSNTKVD | |
| | KKVEPKSCDKTHTCPPCPAPELLGGPSVFLFPPKPKDTLMIS | |
| | RTPEVTCVVVDVSHEDPEVKFNWYVDGVEVHNAKTKPREEQY | |
| | NSTYRVVSVLTVLHQDWLNGKEYKCKVSNKALPAPIEKTISK | |
| | AKGQPREPQVYTLPPCRDELTKNQVSLWCLVKGFYPSDIAVE | |
| | WESNGQPENNYKTTPPVLDSDGSFFLYSKLTVDKSRWQQGNV | |
| | FSCSVMHEALHNRFTQKSLSLSPG | |
| Light | LC: | SEQ ID NO: 63 |
| chain B | DIVLTQSPASLAVSLGQRATISCRASESVDSYGQSYMHWYQQ | |
| | KAGQPPKLLIYYLASNLESGVPARFSGSGSRTDFTLTIDPVQAED | |
| | AATYYCQQNAEDSRTFGGGTKLEIKGGSGSSGSGGDIQMTQS | |
| | PASLSVSVGDTITLTCHASQNIDVWLSWFQQKPGNIPKLLIY | |
| | KASNLHTGVPSRFSGSGSGTGFTLTISSLQPEDIATYYC | |
| | QQAHSYPFTFGGGTKLEIKGGSGSSGSGGRTVAAPSVFIFPP | |
| | SDEQLKSGTASVVCLLNNFYPREAKVQWKVDNALQSGNSQES | |
| | VTEQDSKDSTYSLSSTLTLSKADYEKHKVYACEVTHQGLSSP | |
| | VTKSFNRGEC | |

Binding Protein 19 Nucleotide Sequences

| | | |
|---|---|---|
| Heavy | HC: | SEQ ID NO: 89 |
| chain A | caggtgcagctgcagcagagcggccccgagctggtgaagccc | |
| | ggcgccagcgtgaagatcagctgcaaggccagcggctacagc | |
| | ttcaccagctactggattcactggatcaagcagcgccccggc | |
| | cagggcctggagtggatcggcatgatcgaccccagcgacggc | |
| | gagacccgcctgaaccagcgcttccagggccgcgccaccctg | |
| | accgtggacgagagcaccagcaccgcctacatgcagctgcgc | |
| | agccccaccagcgaggacagcgccgtgtactactgcacccgc | |
| | ctgaaggagtacggcaactacgacagcttctacttcgacgtg | |
| | tggggcgccggcaccctggtgaccgtgagcagcgccagcacc | |
| | aagggccccagcgtgttccccctggcccctagcagcaagagc | |
| | acatctggcggcacagccgccctgggctgcctggtcaaggac | |
| | tacttccccgagcccgtgacagtgtcctggaactctggcgcc | |
| | ctgaccagcggagtgcataccttccctgccgtgctgcagtcc | |
| | agcggcctgtacagcctgagcagcgtggtcacagtgcccagc | |
| | agcagcctgggcacccagacctacatctgcaacgtgaaccac | |
| | aagcccagcaacaccaaggtggacaagaaggtggaacccaag | |
| | agctgcgacaagacccacacctgtcccccctgccctgcccct | |
| | gaactgctgggcggaccctccgtgttcctgttccccccaaag | |
| | cccaaggacaccctgatgatcagccggacccccgaagtgacc | |
| | tgcgtggtggtggacgtgtcccacgaggaccctgaagtgaag | |
| | ttcaattggtacgtggacggcgtggaagtgcataacgccaag | |
| | accaagcccagagaggaacagtacaacagcacctaccgggtg | |
| | gtgtccgtgctgaccgtgctgcaccaggactggctgaacggc | |
| | aaagagtacaagtgcaaggtgtccaacaaggccctgcctgcc | |
| | cccatcgagaaaaccatcagcaaggccaagggccagcctaga | |
| | gagccccaagtctgcaccctgccccccagcagagatgagctg | |
| | accaagaaccaggtgtccctgagctgcgccgtgaagggcttc | |
| | taccccagcgatatcgccgtggaatgggagagcaacggccag | |
| | cccgagaacaactacaagaccaccccccctgtgctggacagc | |
| | gacggctcattcttcctggtgtccaagctgacagtggacaag | |
| | agccggtggcagcagggcaacgtgttcagctgcagcgtgatg | |
| | cacgaggccctgcacaaccattacacccagaagtccctgagc | |
| | ctgagccccggc | |
| Light | LC: | SEQ ID NO: 90 |
| chain A | gacatccagatgacccagagccccgccagcctgagcgtgtcc | |
| | gtgggcgataccatcaccctgacctgccacgccagccagaac | |
| | atcgacgtgtggctgagctggttccagcagaagcccggcaac | |
| | atccccaagctgctgatctacaaggccagcaacctgcacacc | |
| | ggcgtgcccagcagattcagcggctctggcagcggcaccggc | |
| | tttaccctgaccatcagcagcctgcagcccgaggatatcgcc | |
| | acctactactgccagcaggcccacagctaccccttcaccttc | |
| | ggcggaggcaccaagctggaaatcaagggacagcccaaggct | |
| | gccccctcggtcaccctgttccccccaagctctgaggaactg | |
| | caggccaacaaggccaccctcgtgtgcctgatcagcgacttc | |
| | taccctggcgccgtgaccgtggcctggaaggccgatagctct | |
| | cccgtgaaggccggcgtggaaaccaccacccccagcaagcag | |
| | agcaacaacaaatacgccgccagcagctacctgagcctgacc | |
| | cccgagcagtggaagtcccaccggtcctacagctgccaggtc | |
| | acacacgagggcagcaccgtggaaaagaccgtggcccccacc | |
| | gagtgcagc | |
| Heavy | HC: | SEQ ID NO: 66 |
| chain B | caggtgcagctgcagcagagcggccctgagctggtcaagcct | |
| | ggcgccagcgtgaagatcagctgcaaggccagcggctacagc | |
| | ttcaccagctactggatccactggatcaagcagcggcctggc | |
| | cagggcctggaatggatcggcatgatcgaccccagcgacggc | |
| | gagacacggctgaaccagagattccagggcagagccaccctg | |
| | accgtggacgagagcaccagcaccgcctacatgcagctgcgg | |
| | agccccaccagcgaggacagcgccgtgtactactgcacccgg | |
| | ctgaaagagtacggcaactacgacagatctacttcgacgtgt | |
| | ggggagccggcaccctggtcaccgtgtccagcgaagtgcagc | |
| | tgaaagaaagcggccctggcctggtggcccctggcggcagcc | |
| | tgagcatcacctgtaccgtgtccggcttcagcctgaccgaca | |
| | gcagcatcaactgggtccgacagccccctggcaagggcctcg | |
| | agtggctgggaatgatctggggcgacggccggatcgactacg | |
| | ccgacgccctgaagtcccggctgagcatcagcaaggacagca | |
| | gcaagagccaggtgttcctggaaatgaccagcctgcggaccg | |
| | acgacaccgccacctactactgcgccagggacggctacttcc | |
| | cctacgccatggatttctggggccagggcaccagcgtgaccg | |
| | tgtcctctgcttccaccaagggccccagcgtgttccctctgg | |
| | cccctagcagcaagagcacatctggcggaacagccgccctgg | |
| | gctgcctggtcaaggactactttcccgagcccgtgaccgtgt | |
| | cctggaactctggtgccctgacaagcggagtgcataccttcc | |
| | ctgccgtgctgcagagcagcggcctgtactctctgagcagcg | |
| | tggtcaccgtgccaagcagcagcctgggcacccagacctaca | |
| | tctgcaacgtgaaccacaagccctccaacaccaaggtggaca | |
| | agaaggtggaacccaagagctgcgacaagacccacacctgtc | |
| | ctccctgtcctgcccctgaactgctgggcggaccctccgtgt | |
| | tcctgttccctccaaagcccaaggataccctgatgatcagcc | |
| | ggacccctgaagtgacctgcgtggtggtggacgtgtcccacg | |
| | aggatcccgaagtgaagttcaattggtacgtggacggcgtgg | |
| | aagtgcataacgccaagaccaagcccagagaggaacagtaca | |
| | acagcacctaccgggtggtgtccgtgctgacagtgctgcacc | |
| | aggactggctgaacggcaaagagtacaagtgcaaggtgtcca | |
| | acaaggccctgccagcccctatcgagaaaaccatcagcaagg | |
| | ccaagggccagccccgcgagcctcaggtgtacacactgcctc | |
| | catgccgggacgagctgaccaagaaccaggtgtccctgtggt | |
| | gcctcgtgaagggcttctacccctccgatatcgccgtggaat | |
| | gggagagcaacggccagcccgagaacaactacaagaccaccc | |
| | ctcccgtgctggacagcgacggctcattcttcctgtacagca | |
| | agctgaccgtggacaagtcccggtggcagcagggcaacgtgt | |
| | tcagctgctctgtgatgcacgaggccctgcacaaccggttca | |
| | cccagaagtccctgagcctgagccctggc | |
| Light | LC: | SEQ ID NO: 67 |
| chain B | gacatcgtgctgacccagagccctgccagcctggccgtgtct | |
| | ctgggccagagagccaccatcagctgccgggccagcgagagc | |
| | gtggacagctacggccagagctacatgcactggtatcagcag | |
| | aaggccggccagccccccaagctgctgatctacctggccagc | |
| | aacctggaaagcggcgtgcccgccagattcagcggcagcggc | |
| | agcagaaccgacttcaccctgaccatcgaccccgtgcaggcc | |
| | gaggacgccgccacctactactgccagcagaacgccgaggac | |
| | agccggaccttcggcggaggcaccaagctggaaatcaagggc | |
| | ggctccggcagcagcggctctggcggcgatatccagatgacc | |
| | cagtcccccgcctccctgagcgtgtccgtgggcgacaccatc | |
| | accctgacatgccacgccagccagaacatcgacgtgtggctg | |
| | agctggttccagcagaagcctggcaacatccctaagctgctc | |
| | atctataaggcctccaacctgcacaccggcgtgcccagcagg | |
| | ttttccggctctggcagcggcaccggctttaccctgacaatc | |
| | agcagcctgcagcccgaggatatcgccacatattactgtcag | |
| | caggcccacagctaccccttcacctttggcggcggaacaaag | |
| | ctcgagattaagggcggcagcggaagctccggctccggcgga | |
| | cgtacggtggccgctccttccgtgttcatcttccctccctcc | |
| | gacgagcagctgaagtccggcaccgcctccgtggtgtgtctg | |
| | ctgaacaacttctaccctcgggaggccaaggtgcagtggaag | |
| | gtggacaacgccctgcagtccggcaactcccaggagtccgtc | |
| | accgagcaggactccaaggacagcacctactccctgtcctcc | |
| | accctgaccctgtccaaggccgactacgagaagcacaaggtg | |
| | tacgcctgtgaggtgacccaccagggcctgtccagccctgtg | |
| | accaagtccttcaaccggggcgagtgc | |

TABLE 4
CDR sequences of binding proteins

| Ab | CDR_H1 | CDR_H2 | CDR_H3 | CDR_L1 | CDR_L2 | CDR_L3 |
|---|---|---|---|---|---|---|
| Anti- | GFNIKDTY | IYPTNGYT | SRWGGDGFYAMDY | QDVNTA | SAS | QQHYTTPPT |
| Her2 | (SEQ ID NO: 25) | (SEQ ID NO: 26) | (SEQ ID NO: 27) | (SEQ ID NO: 43) | (SEQ ID | (SEQ ID |
| | | | | | NO: 44) | NO: 45) |
| Anti- | GFTFTKAW | IKDKSNS | RGVYYALSPFDY | QSLVHNNANTY | KVS | GQGTQYP |
| CD3 | (SEQ ID NO: 34) | (SEQ ID NO: 35) | (SEQ ID NO: 36) | (SEQ ID NO: 52) | (SEQ ID | (SEQ ID |
| | | | | | NO: 53) | NO: 54) |
| Anti- | GFTFTKAW | IKDKSNS | RGVYYALSPFDY | QSLVHNNGNTY | KVS | GQGTQYP |
| CD3-2 | (SEQ ID NO: 34) | (SEQ ID NO: 35) | (SEQ ID NO: 36) | (SEQ ID NO: 149) | (SEQ ID | (SEQ ID |
| | | | | | NO: 53) | NO: 54) |
| Anti- | GYAFSSYW | IWPGDGDT | ARRETTTVGRYYYAMD | QSVDYDGDSY | DAS | QQSIEDPWT |
| CD19 | (SEQ ID NO: 37) | (SEQ ID NO: 38) | (SEQ ID NO: 39) | (SEQ ID NO: 55) | (SEQ ID | (SEQ ID |
| | | | | | NO: 56) | NO: 57) |
| Anti- | GYTFTSYN | IYPGNGDT | ARSTYYGGDWYFNV | SSVSY | ATS | QQWTSNP |
| CD20 | (SEQ ID NO: 120) | (SEQ ID NO: 121) | (SEQ ID NO: 122) | (SEQ ID NO: 123) | (SEQ ID | (SEQ ID |
| | | | | | NO: 124) | NO: 125) |
| Anti- | GYTFTSYY | IYPGNVNT | TRSHYGLDWNFDV | QNIYVW | KAS | QQGQTYPYT |
| CD28- | (SEQ ID NO: 28) | (SEQ ID NO: 29) | (SEQ ID NO: 30) | (SEQ ID NO: 46) | (SEQ ID | (SEQ ID |
| 1 | | | | | NO: 47) | NO: 48) |
| Anti- | GFSLSDYG | IWAGGGT | ARDKGYSYYYSMD | ESVEYYVTSL | AAS | QQSRKVPYT |
| CD28- | (SEQ ID NO: 31) | (SEQ ID NO: 32) | (SEQ ID NO: 33) | (SEQ ID NO: 49) | (SEQ ID | (SEQ ID |
| 2 | | | | | NO: 50) | NO: 51) |
| Anti- | GYTFTDYW | IYPGDGDT | ARGDYYGSNSLDY | QDVSTV | SAS | QQHYSPPYT |
| CD38 | (SEQ ID NO: 40) | (SEQ ID NO: 41) | (SEQ ID NO: 42) | (SEQ ID NO: 58) | (SEQ ID | (SEQ ID |
| | | | | | NO: 44) | NO: 59) |
| Anti- | GYIFTNYNIH | AIYPGNGDAP | ANWDVAFAY | KASQDIDRYMA | DTSTLQS | LQYDNLWT |
| LAMP1 | (SEQ ID NO: 126) | (SEQ ID NO: 127) | (SEQ ID NO: 128) | (SEQ ID NO: 138) | (SEQ ID | (SEQ ID |
| | | | | | NO: 139) | NO: 140) |
| Anti- | GFTFDDYAMH | AITWNSGHID | VSYLSTASSLDY | RASQGIRNYLA | AASTLQS | QRYNRAPYT |
| TNFα | (SEQ ID NO: 129) | (SEQ ID NO: 130) | (SEQ ID NO: 131) | (SEQ ID NO: 141) | (SEQ ID | (SEQ ID |
| | | | | | NO: 178) | NO: 142) |
| Anti- | GYSFTSYWIH | MIDPSDGET | LKEYGNYDSFYFDV | HASQNIDVWLS | KASNLHT | QQAHSYPFT |
| IL4 | (SEQ ID NO: 132) | (SEQ ID NO: 133) | (SEQ ID NO: 134) | (SEQ ID NO: 143) | (SEQ ID | (SEQ ID |
| | | | | | NO: 179) | NO: 144) |
| Anti- | GFSLTDSSIN | MIWGDGRID | DGYFPYAMDF | RASESVDSYGQSYMH | LASNLES | QQNAEDSRT |
| IL13 | (SEQ ID NO: 135) | (SEQ ID NO: 136) | (SEQ ID NO: 137) | (SEQ ID NO: 145) | (SEQ ID | (SEQ ID |
| | | | | | NO: 146) | NO: 147) |

TABLE 5
VH/VL sequences of binding proteins

| Ab | VH (protein) | VL (protein) |
|---|---|---|
| Anti- | EVQLVESGGGLVQPGGSLRLSCAAS [symbol] | DIQMTQSPSSLSASVGDRVTITCRAS [symbol] |
| Her2 | [symbol] IHWVRQAPGKGLEWVAR [symbol] RYADS | [symbol] VAWYQQKPGKAPKLLIYSASFLY |
| | VKGRFTISADTSKNTAYLQMNSLRAEDTAVY | SGVPSRFSGSRSGTDFTLTISSLQPEDFA |
| | YC [symbol] WGQGTLVTVSS | TYYC [symbol] FGQGTKVEIK |
| | (SEQ ID NO: 150) | (SEQ ID NO: 151) |
| Anti- | QVQLVESGGGVVQPGRSLRLSCAAS [symbol] | DIVMTQTPLSLSVTPGQPASISCKSS [symbol] |
| CD3 | [symbol] MHWVRQAPGKQLEWVAQ [symbol] YATYY | [symbol] LSWYLQKPGQSPQSLIY [symbol] |
| | ADSVKGRFTISRDDSKNTLYLQMNSLRAEDTA | SNRFSGVPDRFSGSGSGTDFTLKISRVE |
| | VYYC [symbol] WGQGTLVTVSS | AEDVGVYYC [symbol] FTFGSGTKVEI |
| | (SEQ ID NO: 152) | K (SEQ ID NO: 153) |
| Anti- | QVQLVESGGGVVQPGRSLRLSCAAS [symbol] | DIVMTQTPLSLSVTPGQPASISCKSS [symbol] |
| CD3-2 | WMHVRQAPGKGLEWVAQ [symbol] NSYATYY | [symbol] LSWYLQKPGQSPQLLIY [symbol] |
| | ADSVKGRFTISRDNSKNTLYLQMNSLRAEDTA | SNRFSGVPDRFSGSGSGTDFTLKISRVE |
| | VYYC [symbol] WGQGTLVTVSS | AEDVGVYYC [symbol] FTFGGGTKVEI |
| | (SEQ ID NO: 154) | K (SEQ ID NO: 155) |
| Anti- | QVQLVQSGAEVKKPGSSVKVSCKAS [symbol] | DLVLTQSPASLAVSPGQRATITCKAS [symbol] |
| CD19 | WMNWVRQAPGQGLEWIGQ [symbol] NYNQ | [symbol] LNWYQQKPGQPPKLLIY [symbol] |
| | KFKGRATLTADESTSTAYMELSSLRSEDTAVY | SNLVSGVPARFSGSGSGTDFTLTINPVE |
| | YC [symbol] YWGQGTTVTVSS | ANDTANYYC [symbol] FGQGTKLEI |
| | (SEQ ID NO: 156) | K (SEQ ID NO: 157) |
| Anti- | QVQLQQPGAELVKPGASVKMSCKAS [symbol] | QIVLSQSPAILSASPGEKVTMTCRAS [symbol] |
| CD20 | YNMHWVKQTPGRGLEWIGA [symbol] SYNQ | [symbol] IHWFQQKPGSSPKPWIYATSNLASGV |
| | KFKGKATLTADKSSSTAYMQLSSLTSEDSAVY | PVRFSGSGSGTSYSLTISRVEAEDAATY |
| | YC [symbol] WGAGTTVTVSA | Y [symbol] PTFGGGTKLEIK |
| | (SEQ ID NO: 158) | (SEQ ID NO: 159) |
| Anti- | QVQLVQSGAEVVKPGASVKVSCKAS [symbol] | DIQMTQSPSSLSASVGDRVTITCQAS [symbol] |
| CD28-1 | [symbol] IHWVRQAPGQGLEWIGS [symbol] NYAQK | [symbol] LNWYQQKPGKAPKLLIY [symbol] NLH |
| | FQGRATLTVDTSISTAYMELSRLRSDDTAVYY | TGVPSRFSGSGSGTDFTLTISSLQPEDIA |
| | C [symbol] WGKGTTVTVSS | TYYC [symbol] FGQGTKLEIK |
| | (SEQ ID NO: 160) | (SEQ ID NO: 161) |
| Anti- | QVQLQESGPGLVKPSQTLSLTCTVS [symbol] | DIVLTQSPASLAVSPGQRATITCRAS [symbol] |
| CD28-2 | VHWVRQPPGKGLEWLGV [symbol] NYNPSL | [symbol] MQWYQQKPGQPPKLLIF [symbol] |
| | KSRKTISKDTSKNQVSLKLSSVTAADTAVYYC | NVESGVPARFSGSGSGTDFTLTINPVEA |
| | [symbol] YWGQGTTVTVSS | NDVANYYC [symbol] FGQGTKLEIK |
| | (SEQ ID NO: 162) | (SEQ ID NO: 163) |
| Anti- | QVQLVQSGAEVAKPGTSVKLSCKAS [symbol] | DIVMTQSHLSMSTSLGDPVSITCKAS [symbol] |
| CD38 | WMQWVKQRPGQGLEWIGT [symbol] GYAQ | [symbol] VAWYQQKPGQSPRRLIYSASYRYI |
| | KFQGKATLTADKSSKTVYMHLSSLASEDSAV | GVPDRFTGSGAGTDFTFTISSVQAEDLA |
| | YYCA [symbol] WGQGTSVTVSS | VYYC [symbol] FGGGTKLEIK |
| | (SEQ ID NO: 164) | (SEQ ID NO: 165) |
| Anti- | QVQLVQSGAEVKKPGSSVKVSCKAS [symbol] | DIQMTQSPSSLSASVGDRVTITC [symbol] |
| LAMP | [symbol] WVKKSPGQGLEWIG [symbol] YSQKF | [symbol] WYQDKPGKAPRLLIH [symbol] |
| 1 | QGKATLTADTSTSTTYMELSSLRSEDTAVYYC | GVPSRFSGSGSGRDYTLTISNLEPEDFAT |
| | VR [symbol] WGQGTLVTVSS (SEQ ID | YYC [symbol] FGGGTKVEIK |
| | NO: 166) | (SEQ ID NO: 167) |
| Anti- | EVQLVESGGGLVQPGRSLRLSCAAS [symbol] | DIQMTQSPSSLSASVGDRVTITC [symbol] |
| TNFα | [symbol] WVRQAPGKGLEWVS [symbol] YAD | [symbol] WYQQKPGKAPKLLIY [symbol] |
| | SVEGRFTISRDNAKNSLYLQMNSLRAEDTAVY | GVPSRFSGSGSGTDFTLTISSLQPEDVAT |
| | YCAK [symbol] WGQGTLVTVSS | YYC [symbol] FGQGTKVEIK |
| | (SEQ ID NO: 168) | (SEQ ID NO: 169) |
| Anti- | QVQLQQSGPELVKPGASVKISCKAS [symbol] | DIQMTQSPASLSVSVGDTITLTC [symbol] |
| IL4 | [symbol] WIKQRPGQGLEWI [symbol] RLNQRF | [symbol] WFQQKPGNIPKLLIY [symbol] |
| | QGRATLTVDESTSTAYMQLRSPTSEDSAVYYC | GVPSRFSGSGSGTGFTLTISSLQPEDIAT |
| | TR [symbol] WGAGTLVTVSS | YYC [symbol] FGGGTKLEIK |
| | (SEQ ID NO: 170) | (SEQ ID NO: 171) |
| Anti- | EVQLKESGPGLVAPGGSLSITCTVS [symbol] | DIVLTQSPASLAVSLGQRATISC [symbol] |
| IL13 | NWVRQPPGKGLEWLG [symbol] YADALK | [symbol] WYQQKAGQPPKLLIY [symbol] |
| | SRLSISKDSSKSQVFLEMTSLRTDDTATYYCAR | [symbol] GVPARFSGSGSRTDFTLTIDPVQA |
| | [symbol] WGQGTSVTVSS | EDAATYYC [symbol] FGGGTKLEIK |
| | (SEQ ID NO: 172) | (SEQ ID NO: 173) |

Note:
CDR sequences are bolded and italicized in amino acid sequences above.

1-90. (canceled)

91: A trivalent and/or trispecific binding protein comprising four polypeptide chains that form three antigen binding sites that specifically bind one or more target proteins, wherein a first polypeptide chain of the binding protein comprises a structure represented by the formula:
V_{L2}-L₁-V_{L1}-L₂-C_{L} [I]
and a second polypeptide chain of the binding protein comprises a structure represented by the formula:
V_{H1}-L₃-V_{H2}-L₄-C_{H1}-hinge-C_{H2}-C_{H3} [II]
and a third polypeptide chain of the binding protein comprises a structure represented by the formula:
V_{H3}-C_{H1}-hinge-C_{H2}-C_{H3} [III]
and a fourth polypeptide chain of the binding protein comprises a structure represented by the formula:
V_{L3}-C_{L} [IV]
wherein:
V_{L}, is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain;
C_{H2} is an immunoglobulin C_{H2} heavy chain constant domain;
C_{H3} is an immunoglobulin C_{H3} heavy chain constant domain;
hinge is an immunoglobulin hinge region connecting the C_{H1} and C_{H2} domains; and
L₁, L₂, L₃ and L₄ are each independently amino acid linkers or zero amino acids in length;
wherein the polypeptide of formula I and the polypeptide of formula II form a cross-over light chain-heavy chain pair of the binding protein;
wherein the polypeptide of formula I and the polypeptide of formula II form a cross-over light chain-heavy chain pair of the binding protein;
wherein V_{H1} and V_{L1} form a binding pair and a first antigen binding site, V_{H2} and V_{L2} form a binding pair and a second antigen binding site, and V_{H3} and V_{L3} form a binding pair and a third antigen binding site; and
wherein the first antigen binding site specifically binds human IL4 or IL13, and the second antigen binding site specifically binds human IL4 or IL13, the first and second antigen binding sites being different.

92: An isolated nucleic acid molecule comprising a nucleotide sequence encoding the binding protein of claim 91.

93: An expression vector comprising the nucleic acid molecule of claim 92.

94: An isolated host cell comprising the nucleic acid molecule of claim 92.

95: An isolated host cell comprising the expression vector of claim 93.

96: A pharmaceutical composition comprising the binding protein of claim 91 and a pharmaceutically acceptable carrier.

97: A method of preventing or treating cancer in a patient comprising administering to the patient a therapeutically effective amount of the pharmaceutical composition of claim 96, wherein the binding protein comprises one antigen binding site that specifically binds a T-cell surface protein and another antigen binding site that specifically binds a target protein.

98: The method of claim 97, wherein the patient is a human.

99: A method of preventing or treating an inflammatory disease or disorder in a patient comprising administering to the patient a therapeutically effective amount of the pharmaceutical composition of claim 96, wherein the binding protein comprises three antigen binding sites that each specifically bind a cytokine target protein.

100: The method of claim 99, wherein the patient is a human.


SEQUENCE LISTING (179 sequences)

SEQ ID NO: 1 - protein, 447 aa, synthetic construct
  REGION 1..447: note = Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLGK

SEQ ID NO: 2 - protein, 214 aa, synthetic construct
  REGION 1..214: note = Synthetic Construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 571 aa, synthetic construct
  REGION 1..571: note = Synthetic Construct
QVQLVQSGAEVVKPGASVKVSCKASGYTFTSYYIHWVRQAPGQGLEWIGSIYPGNVNTNYAQKFQGRATLTVDTSISTAYMELSRLRSDDTAVYYCTRSHYGLDWNFDVWGKGTTVTVSSSQVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSRTASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNHYTQKSLSLSLGK

SEQ ID NO: 4 - protein, 338 aa, synthetic construct
  REGION 1..338: note = Synthetic Construct
DIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIKGQPKAAPDIQMTQSPSSLSASVGDRVTITCQASQNIYVWLNWYQQKPGKAPKLLIYKASNLHTGVPSRFSGSGSGTDFTLTISSLQPEDIATYYCQQGQTYPYTFGQGTKLEIKTKGPSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 1341 bp, synthetic construct
  misc_feature 1..1341: note = Synthetic Construct
  source 1..1341: mol_type = other DNA
gaagtgcagctggtggaatctggcggcggactggtgcagcctggcggatctctgagactgagctgtgccgccagcggcttcaacatcaaggacacctacatccactgggtgcgccaggcccctggcaagggactggaatgggtggccagaatctaccccaccaacggctacaccagatacgccgacagcgtgaagggccggttcaccatcagcgccgacaccagcaagaacaccgcctacctgcagatgaacagcctgcgggccgaggacaccgccgtgtactactgtagtagatggggaggcgacggcttctacgccatggactattggggccagggcaccctcgtgaccgtgtctagtgcgtcgaccaagggcccatcggtgttccctctggccccttgcagcagaagcaccagcgaatctacagccgccctgggctgcctcgtgaaggactactttcccgagcccgtgaccgtgtcctggaactctggcgctctgacaagcggcgtgcacacctttccagccgtgctccagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgaccgtgctgcaccaggactggctgaacggcaaagagtacaagtgcaaggtgtccaacaagggcctgcccagctccatcgagaaaaccatcagcaaggccaagggccagccccgcgagcctcaagtgtataccctgcccccttgccaggaagagatgaccaagaaccaggtgtccctgtggtgtctcgtgaaaggcttctaccccagcgacattgccgtggaatgggagagcaacggccagcccgagaacaactacaagaccaccccccctgtgctggacagcgacggctcattcttcctgtactccaagctgaccgtggacaagagccggtggcaggaaggcaacgtgttcagctgctccgtgatgcacgaggccctgcacaaccactacacccagaagtccctgtctctgtccctgggcaag

SEQ ID NO: 6 - DNA, 642 bp, synthetic construct
  misc_feature 1..642: note = Synthetic Construct
  source 1..642: mol_type = other DNA
gacatccagatgacccagagccccagcagcctgtctgccagcgtgggcgacagagtgaccatcacctgtagagccagccaggacgtgaacaccgccgtggcctggtatcagcagaagcctggcaaggcccccaagctgctgatctacagcgccagcttcctgtacagcggcgtgcccagcagattcagcggaagcagaagcggcaccgacttcaccctgaccatcagctccctgcagcccgaggacttcgccacctactactgccagcagcactacaccaccccccccacatttggccagggcaccaaggtggaaatcaagcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgt

SEQ ID NO: 7 - DNA, 1713 bp, synthetic construct
  misc_feature 1..1713: note = Synthetic Construct
  source 1..1713: mol_type = other DNA
caggtgcagctggtgcagtctggcgccgaggtcgtgaaacctggcgcctctgtgaaggtgtcctgcaaggccagcggctacacctttaccagctactacatccactgggtgcgccaggcccctggacagggactggaatggatcggcagcatctaccccggcaacgtgaacaccaactacgcccagaagttccagggcagagccaccctgaccgtggacaccagcatcagcaccgcctacatggaactgagccggctgagaagcgacgacaccgccgtgtactactgcacccggtcccactacggcctggattggaacttcgacgtgtggggcaagggcaccaccgtgacagtgtctagcagccaggtgcagctggtggaatctggcggcggagtggtgcagcctggcagaagcctgagactgagctgtgccgccagcggcttcaccttcaccaaggcctggatgcactgggtgcgccaggcccctggaaagcagctggaatgggtggcccagatcaaggacaagagcaacagctacgccacctactacgccgacagcgtgaagggccggttcaccatcagccgggacgacagcaagaacaccctgtacctgcagatgaacagcctgcgggccgaggacaccgccgtgtactactgtcggggcgtgtactatgccctgagccccttcgattactggggccagggaaccctcgtgaccgtgtctagtcggaccgccagcacaaagggcccatcggtgttccctctggccccttgcagcagaagcaccagcgaatctacagccgccctgggctgcctcgtgaaggactactttcccgagcccgtgaccgtgtcctggaactctggcgctctgacaagcggcgtgcacacctttccagccgtgctccagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgaccgtgctgcaccaggactggctgaacggcaaagagtacaagtgcaaggtgtccaacaagggcctgcccagctccatcgagaaaaccatcagcaaggccaagggccagccccgcgagcctcaagtgtgtaccctgccccctagccaggaagagatgaccaagaaccaggtgtccctgagctgtgccgtgaaaggcttctaccccagcgacattgccgtggaatgggagagcaacggccagcccgagaacaactacaagaccaccccccctgtgctggacagcgacggctcattcttcctggtgtccaagctgaccgtggacaagagccggtggcaggaaggcaacgtgttcagctgctccgtgatgcacgaggccctgcacaaccactacacccagaagtccctgtctctgtccctgggcaag

SEQ ID NO: 8 - DNA, 1014 bp, synthetic construct
  misc_feature 1..1014: note = Synthetic Construct
  source 1..1014: mol_type = other DNA
gacatcgtgatgacccagacccccctgagcctgagcgtgacacctggacagcctgccagcatcagctgcaagagcagccagagcctggtgcacaacaacgccaacacctacctgagctggtatctgcagaagcccggccagagcccccagtccctgatctacaaggtgtccaacagattcagcggcgtgcccgacagattctccggcagcggctctggcaccgacttcaccctgaagatcagccgggtggaagccgaggacgtgggcgtgtactattgtggccagggcacccagtaccccttcacctttggcagcggcaccaaggtggaaatcaagggccagcccaaggccgcccccgacatccagatgacccagagccccagcagcctgtctgccagcgtgggcgacagagtgaccatcacctgtcaggccagccagaacatctacgtgtggctgaactggtatcagcagaagcccggcaaggcccccaagctgctgatctacaaggccagcaacctgcacaccggcgtgcccagcagattttctggcagcggctccggcaccgacttcaccctgacaatcagctccctgcagcccgaggacattgccacctactactgccagcagggccagacctacccctacacctttggccagggcaccaagctggaaatcaagaccaagggccccagccgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgt

SEQ ID NO: 9 - protein, 571 aa, synthetic construct
  REGION 1..571: note = Synthetic Construct
QVQLQESGPGLVKPSQTLSLTCTVSGFSLSDYGVHWVRQPPGKGLEWLGVIWAGGGTNYNPSLKSRKTISKDTSKNQVSLKLSSVTAADTAVYYCARDKGYSYYYSMDYWGQGTTVTVSSSQVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSRTASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNHYTQKSLSLSLGK

SEQ ID NO: 10 - protein, 342 aa, synthetic construct
  REGION 1..342: note = Synthetic Construct
DIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIKGQPKAAPDIVLTQSPASLAVSPGQRATITCRASESVEYYVTSLMQWYQQKPGQPPKLLIFAASNVESGVPARFSGSGSGTDFTLTINPVEANDVANYYCQQSRKVPYTFGQGTKLEIKTKGPSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 1713 bp, synthetic construct
  misc_feature 1..1713: note = Synthetic Construct
  source 1..1713: mol_type = other DNA
caggtgcagctgcaggaatctggccctggcctcgtgaagcctagccagaccctgagcctgacctgtaccgtgtccggcttcagcctgagcgactacggcgtgcactgggtgcgccagccacctggaaaaggcctggaatggctgggcgtgatctgggctggcggaggcaccaactacaaccccagcctgaagtccagaaagaccatcagcaaggacaccagcaagaaccaggtgtccctgaagctgagcagcgtgacagccgccgataccgccgtgtactactgcgccagagacaagggctacagctactactacagcatggactactggggccagggcaccaccgtgaccgtgtcatcctctcaggtgcagctggtggaatctggcggcggagtggtgcagcctggcagaagcctgagactgagctgtgccgccagcggcttcaccttcaccaaggcctggatgcactgggtgcgccaggcccctggaaagcagctggaatgggtggcccagatcaaggacaagagcaacagctacgccacctactacgccgacagcgtgaagggccggttcaccatcagccgggacgacagcaagaacaccctgtacctgcagatgaacagcctgcgggccgaggacaccgccgtgtactactgtcggggcgtgtactatgccctgagccccttcgattactggggccagggaaccctcgtgaccgtgtctagtcggaccgcttcgaccaagggcccatcggtgttccctctggccccttgcagcagaagcaccagcgaatctacagccgccctgggctgcctcgtgaaggactactttcccgagcccgtgaccgtgtcctggaactctggcgctctgacaagcggcgtgcacacctttccagccgtgctccagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgaccgtgctgcaccaggactggctgaacggcaaagagtacaagtgcaaggtgtccaacaagggcctgcccagctccatcgagaaaaccatcagcaaggccaagggccagccccgcgagcctcaagtgtgtaccctgccccctagccaggaagagatgaccaagaaccaggtgtccctgagctgtgccgtgaaaggcttctaccccagcgacattgccgtggaatgggagagcaacggccagcccgagaacaactacaagaccaccccccctgtgctggacagcgacggctcattcttcctggtgtccaagctgaccgtggacaagagccggtggcaggaaggcaacgtgttcagctgctccgtgatgcacgaggccctgcacaaccactacacccagaagtccctgtctctgtccctgggcaag

SEQ ID NO: 12 - DNA, 1026 bp, synthetic construct
  misc_feature 1..1026: note = Synthetic Construct
  source 1..1026: mol_type = other DNA
gacatcgtgatgacccagacccccctgagcctgagcgtgacacctggacagcctgccagcatcagctgcaagagcagccagagcctggtgcacaacaacgccaacacctacctgagctggtatctgcagaagcccggccagagcccccagtccctgatctacaaggtgtccaacagattcagcggcgtgcccgacagattctccggcagcggctctggcaccgacttcaccctgaagatcagccgggtggaagccgaggacgtgggcgtgtactattgtggccagggcacccagtaccccttcacctttggcagcggcaccaaggtggaaatcaagggccagcccaaggccgcccccgacatcgtgctgacacagagccctgctagcctggccgtgtctcctggacagagggccaccatcacctgtagagccagcgagagcgtggaatattacgtgaccagcctgatgcagtggtatcagcagaagcccggccagccccccaagctgctgattttcgccgccagcaacgtggaaagcggcgtgccagccagattttccggcagcggctctggcaccgacttcaccctgaccatcaaccccgtggaagccaacgacgtggccaactactactgccagcagagccggaaggtgccctacacctttggccagggcaccaagctggaaatcaagaccaagggccccagccgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgt

SEQ ID NO: 13 - protein, 451 aa, synthetic construct
  REGION 1..451: note = Synthetic Construct
QVQLVQSGAEVKKPGSSVKVSCKASGYAFSSYWMNWVRQAPGQGLEWIGQIWPGDGDTNYNQKFKGRATLTADESTSTAYMELSSLRSEDTAVYYCARRETTTVGRYYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLGK

SEQ ID NO: 14 - protein, 218 aa, synthetic construct
  REGION 1..218: note = Synthetic Construct
DLVLTQSPASLAVSPGQRATITCKASQSVDYDGDSYLNWYQQKPGQPPKLLIYDASNLVSGVPARFSGSGSGTDFTLTINPVEANDTANYYCQQSTEDPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - DNA, 1353 bp, synthetic construct
  misc_feature 1..1353: note = Synthetic Construct
  source 1..1353: mol_type = other DNA
caggtgcagctggtgcagagcggcgccgaagtgaagaagcctggcagcagcgtgaaggtgagctgcaaggccagcggctatgccttcagcagctactggatgaactgggtgaggcaggcacctggccagggcctggagtggataggccaaatatggcctggcgatggcgacaccaactacaaccagaagttcaagggcagagcgaccttgaccgccgacgagagcaccagcaccgcgtacatggagctgagcagcctgaggagcgaggacaccgccgtgtactattgcgccagaagggagaccaccaccgtgggcaggtactactacgccatggactactggggccagggaaccaccgtgaccgtgagcagcgcctcgaccaagggcccatcggtgttccctctggccccttgcagcagaagcaccagcgaatctacagccgccctgggctgcctcgtgaaggactactttcccgagcccgtgaccgtgtcctggaactctggcgctctgacaagcggcgtgcacacctttccagccgtgctccagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgaccgtgctgcaccaggactggctgaacggcaaagagtacaagtgcaaggtgtccaacaagggcctgcccagctccatcgagaaaaccatcagcaaggccaagggccagccccgcgagcctcaagtgtataccctgcccccttgccaggaagagatgaccaagaaccaggtgtccctgtggtgtctcgtgaaaggcttctaccccagcgacattgccgtggaatgggagagcaacggccagcccgagaacaactacaagaccaccccccctgtgctggacagcgacggctcattcttcctgtactccaagctgaccgtggacaagagccggtggcaggaaggcaacgtgttcagctgctccgtgatgcacgaggccctgcacaaccactacacccagaagtccctgtctctgtccctgggcaag

SEQ ID NO: 16 - DNA, 654 bp, synthetic construct
  misc_feature 1..654: note = Synthetic Construct
  source 1..654: mol_type = other DNA
gacctcgtgctgacccagagccctgcgagcctggctgtgagccctggccagagagccaccatcacctgcaaagccagccagagcgtggactacgacggcgacagctacctcaactggtaccagcagaagcctggccagccccccaagctgctgatttacgatgccagcaacctggtgagcggcgtgcctgctagattcagcggctccggcagcggcaccgacttcaccctgaccatcaaccccgtggaggccaacgacaccgccaactactactgccagcagagcacggaggacccctggaccttcggccagggcacaaagctggagatcaagcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgt

SEQ ID NO: 17 - protein, 447 aa, synthetic construct
  REGION 1..447: note = Synthetic Construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLGK

SEQ ID NO: 18 - protein, 214 aa, synthetic construct
  REGION 1..214: note = Synthetic Construct
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - DNA, 1341 bp, synthetic construct
  misc_feature 1..1341: note = Synthetic Construct
  source 1..1341: mol_type = other DNA
caggtgcagctggtgcagtctggcgccgaagtggccaagcctggcacaagcgtgaagctgagctgcaaggccagcggctacaccttcaccgactactggatgcagtgggtcaagcagaggccaggccagggcctggaatggatcggcacaatctatcccggcgacggcgataccggctacgcccagaagtttcagggcaaggccaccctgaccgccgacaagagcagcaagaccgtgtacatgcacctgagcagcctggccagcgaggacagcgccgtgtactattgcgccagaggcgactactacggcagcaacagcctggactattggggccagggcaccagcgtgacagtgtctagtgcgtcgaccaagggcccatcggtgttccctctggccccttgcagcagaagcaccagcgaatctacagccgccctgggctgcctcgtgaaggactactttcccgagcccgtgaccgtgtcctggaactctggcgctctgacaagcggcgtgcacacctttccagccgtgctccagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgaccgtgctgcaccaggactggctgaacggcaaagagtacaagtgcaaggtgtccaacaagggcctgcccagctccatcgagaaaaccatcagcaaggccaagggccagccccgcgagcctcaagtgtataccctgcccccttgccaggaagagatgaccaagaaccaggtgtccctgtggtgtctcgtgaaaggcttctaccccagcgacattgccgtggaatgggagagcaacggccagcccgagaacaactacaagaccaccccccctgtgctggacagcgacggctcattcttcctgtactccaagctgaccgtggacaagagccggtggcaggaaggcaacgtgttcagctgctccgtgatgcacgaggccctgcacaaccactacacccagaagtccctgtctctgtccctgggcaag

SEQ ID NO: 20 - DNA, 642 bp, synthetic construct
  misc_feature 1..642: note = Synthetic Construct
  source 1..642: mol_type = other DNA
gacatcgtgatgacccagagccacctgagcatgagcaccagcctgggcgaccccgtgtccatcacctgtaaagccagccaggacgtgtccaccgtggtggcctggtatcagcagaagcctggccagagccccagacggctgatctacagcgccagctatcggtacatcggcgtgcccgacagattcaccggaagcggagccggcaccgacttcaccttcaccatcagctctgtgcaggccgaggacctggccgtgtactactgccagcagcactacagccccccctacacctttggcggaggcaccaagctggaaatcaagcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgt

SEQ ID NO: 21 - protein, 445 aa, synthetic construct
  REGION 1..445: note = Synthetic Construct
QVQLVQSGAEVKKPGSSVKVSCKASGYIFTNYNIHWVKKSPGQGLEWIGAIYPGNGDAPYSQKFQGKATLTADTSTSTTYMELSSLRSEDTAVYYCVRANWDVAFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLGK

SEQ ID NO: 22 - protein, 213 aa, synthetic construct
  REGION 1..213: note = Synthetic Construct
DIQMTQSPSSLSASVGDRVTITCKASQDIDRYMAWYQDKPGKAPRLLIHDTSTLQSGVPSRFSGSGSGRDYTLTISNLEPEDFATYYCLQYDNLWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - DNA, 1335 bp, synthetic construct
  misc_feature 1..1335: note = Synthetic Construct
  source 1..1335: mol_type = other DNA
caggtgcagctggtgcagtctggcgccgaagtgaagaaacccggcagcagcgtgaaggtgtcctgcaaggccagcggctacatcttcaccaactacaacatccactgggtcaagaagtccccaggccagggcctggaatggatcggcgccatctatcccggaaacggcgacgccccttacagccagaagttccagggcaaggccaccctgaccgccgataccagcacctccaccacctacatggaactgagcagcctgcggagcgaggacaccgccgtgtactattgcgtgcgggccaactgggatgtggccttcgcctattggggccagggcacactcgtgaccgtgtcctctgcgtcgaccaagggcccatcggtgttccctctggccccttgcagcagaagcaccagcgaatctacagccgccctgggctgcctcgtgaaggactactttcccgagcccgtgaccgtgtcctggaactctggcgctctgacaagcggcgtgcacacctttccagccgtgctccagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgaccgtgctgcaccaggactggctgaacggcaaagagtacaagtgcaaggtgtccaacaagggcctgcccagctccatcgagaaaaccatcagcaaggccaagggccagccccgcgagcctcaagtgtataccctgcccccttgccaggaagagatgaccaagaaccaggtgtccctgtggtgtctcgtgaaaggcttctaccccagcgacattgccgtggaatgggagagcaacggccagcccgagaacaactacaagaccaccccccctgtgctggacagcgacggctcattcttcctgtactccaagctgaccgtggacaagagccggtggcaggaaggcaacgtgttcagctgctccgtgatgcacgaggccctgcacaaccactacacccagaagtccctgtctctgtccctgggcaag

SEQ ID NO: 24 - DNA, 639 bp, synthetic construct
  misc_feature 1..639: note = Synthetic Construct
  source 1..639: mol_type = other DNA
gacatccagatgacccagagccccagcagcctgtctgccagcgtgggcgacagagtgaccatcacatgcaaggccagccaggacatcgatcggtacatggcctggtatcaggacaagcccggcaaggcccccagactgctgatccacgataccagcacactgcagagcggcgtgcccagcagattttccggctctggcagcggcagagactacaccctgaccatcagcaacctggaacccgaggacttcgccacctactactgcctgcagtacgacaacctgtggaccttcggcggaggcaccaaggtggaaatcaagcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgt

SEQ ID NO: 25 - protein, 8 aa, synthetic construct
  REGION 1..8: note = Synthetic Construct
GFNIKDTY

SEQ ID NO: 26 - protein, 8 aa, synthetic construct
  REGION 1..8: note = Synthetic Construct
IYPTNGYT

SEQ ID NO: 27 - protein, 13 aa, synthetic construct
  REGION 1..13: note = Synthetic Construct
SRWGGDGFYAMDY

SEQ ID NO: 28 - protein, 8 aa, synthetic construct
  REGION 1..8: note = Synthetic Construct
GYTFTSYY

SEQ ID NO: 29 - protein, 8 aa, synthetic construct
  REGION 1..8: note = Synthetic Construct
IYPGNVNT

SEQ ID NO: 30 - protein, 13 aa, synthetic construct
  REGION 1..13: note = Synthetic Construct
TRSHYGLDWNFDV

SEQ ID NO: 31 - protein, 8 aa, synthetic construct
  REGION 1..8: note = Synthetic Construct
GFSLSDYG

SEQ ID NO: 32 - protein, 7 aa, synthetic construct
  REGION 1..7: note = Synthetic Construct
IWAGGGT

SEQ ID NO: 33 - protein, 13 aa, synthetic construct
  REGION 1..13: note = Synthetic Construct
ARDKGYSYYYSMD

SEQ ID NO: 34 - protein, 8 aa, synthetic construct
  REGION 1..8: note = Synthetic Construct
GFTFTKAW

SEQ ID NO: 35 - protein, 7 aa, synthetic construct
  REGION 1..7: note = Synthetic Construct
IKDKSNS

SEQ ID NO: 36 - protein, 12 aa, synthetic construct
  REGION 1..12: note = Synthetic Construct
RGVYYALSPFDY

SEQ ID NO: 37 - protein, 8 aa, synthetic construct
  REGION 1..8: note = Synthetic Construct
GYAFSSYW

SEQ ID NO: 38 - protein, 8 aa, synthetic construct
  REGION 1..8: note = Synthetic Construct
IWPGDGDT

SEQ ID NO: 39 - protein, 16 aa, synthetic construct
  REGION 1..16: note = Synthetic Construct
ARRETTTVGRYYYAMD

SEQ ID NO: 40 - protein, 8 aa, synthetic construct
  REGION 1..8: note = Synthetic Construct
GYTFTDYW

SEQ ID NO: 41 - protein, 8 aa, synthetic construct
  REGION 1..8: note = Synthetic Construct
IYPGDGDT

SEQ ID NO: 42 - protein, 13 aa, synthetic construct
  REGION 1..13: note = Synthetic Construct
ARGDYYGSNSLDY

SEQ ID NO: 43 - protein, 6 aa, synthetic construct
  REGION 1..6: note = Synthetic Construct
QDVNTA

SEQ ID NO: 44 - skipped

SEQ ID NO: 45 - protein, 9 aa, synthetic construct
  REGION 1..9: note = Synthetic Construct
QQHYTTPPT

SEQ ID NO: 46 - protein, 6 aa, synthetic construct
  REGION 1..6: note = Synthetic Construct
QNIYVW

SEQ ID NO: 47 - skipped

SEQ ID NO: 48 - protein, 9 aa, synthetic construct
  REGION 1..9: note = Synthetic Construct
QQGQTYPYT

SEQ ID NO: 49 - protein, 10 aa, synthetic construct
  REGION 1..10: note = Synthetic Construct
ESVEYYVTSL

SEQ ID NO: 50 - skipped

SEQ ID NO: 51 - protein, 9 aa, synthetic construct
  REGION 1..9: note = Synthetic Construct
QQSRKVPYT

SEQ ID NO: 52 - protein, 11 aa, synthetic construct
  REGION 1..11: note = Synthetic Construct
QSLVHNNANTY

SEQ ID NO: 53 - skipped

SEQ ID NO: 54 - protein, 7 aa, synthetic construct
  REGION 1..7: note = Synthetic Construct
GQGTQYP

SEQ ID NO: 55 - protein, 10 aa, synthetic construct
  REGION 1..10: note = Synthetic Construct
QSVDYDGDSY

SEQ ID NO: 56 - skipped

SEQ ID NO: 57 - protein, 9 aa, synthetic construct
  REGION 1..9: note = Synthetic Construct
QQSTEDPWT

SEQ ID NO: 58 - protein, 6 aa, synthetic construct
  REGION 1..6: note = Synthetic Construct
QDVSTV

SEQ ID NO: 59 - protein, 9 aa, synthetic construct
  REGION 1..9: note = Synthetic Construct
QQHYSPPYT

SEQ ID NO: 60 - protein, 450 aa, synthetic construct
  REGION 1..450: note = Synthetic Construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 61 - protein, 213 aa, synthetic construct
  REGION 1..213: note = Synthetic Construct
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 62 - protein, 570 aa, synthetic construct
  REGION 1..570: note = Synthetic Construct
QVQLQQSGPELVKPGASVKISCKASGYSFTSYWIHWIKQRPGQGLEWIGMIDPSDGETRLNQRFQGRATLTVDESTSTAYMQLRSPTSEDSAVYYCTRLKEYGNYDSFYFDVWGAGTLVTVSSEVQLKESGPGLVAPGGSLSITCTVSGFSLTDSSINWVRQPPGKGLEWLGMIWGDGRIDYADALKSRLSISKDSSKSQVFLEMTSLRTDDTATYYCARDGYFPYAMDFWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 63 - protein, 345 aa, synthetic construct
  REGION 1..345: note = Synthetic Construct
DIVLTQSPASLAVSLGQRATISCRASESVDSYGQSYMHWYQQKAGQPPKLLIYLASNLESGVPARFSGSGSRTDFTLTIDPVQAEDAATYYCQQNAEDSRTFGGGTKLEIKGGSGSSGSGGDIQMTQSPASLSVSVGDTITLTCHASQNIDVWLSWFQQKPGNIPKLLIYKASNLHTGVPSRFSGSGSGTGFTLTISSLQPEDIATYYCQQAHSYPFTFGGGTKLEIKGGSGSSGSGGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - DNA, 1350 bp, synthetic construct
  misc_feature 1..1350: note = Synthetic Construct
  source 1..1350: mol_type = other DNA
gaggtgcagctggtggaaagcggcggaggactggtgcagcccggcagaagcctgagactgagctgcgccgccagcggcttcaccttcgacgactacgccatgcactgggtccgccaggcccctggcaagggcctggaatgggtgtccgccatcacctggaacagcggccacatcgactacgccgacagcgtggaaggccggttcaccatcagccgggacaacgccaagaacagcctgtacctgcagatgaacagcctgcgggccgaggacaccgccgtgtactactgcgccaaggtgtcctacctgagcaccgccagcagcctggactactggggccagggcaccctggtgacagtgtccagcgcttccaccaagggccccagcgtgttccccctggcccctagcagcaagagcacatctggcggcacagccgccctgggctgcctggtcaaggactacttccccgagcccgtgacagtgtcctggaactctggcgccctgaccagcggagtgcataccttccctgccgtgctgcagtccagcggcctgtacagcctgagcagcgtggtcacagtgcccagcagcagcctgggcacccagacctacatctgcaacgtgaaccacaagcccagcaacaccaaggtggacaagaaggtggaacccaagagctgcgacaagacccacacctgtcccccctgccctgcccctgaactgctgggcggaccctccgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcataacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaagggccagcctagagagccccaagtctgcaccctgccccccagcagagatgagctgaccaagaaccaggtgtccctgagctgcgccgtgaagggcttctaccccagcgatatcgccgtggaatgggagagcaacggccagcccgagaacaactacaagaccaccccccctgtgctggacagcgacggctcattcttcctggtgtccaagctgacagtggacaagagccggtggcagcagggcaacgtgttcagctgcagcgtgatgcacgaggccctgcacaaccattacacccagaagtccctgagcctgagccccggc

SEQ ID NO: 65 - DNA, 639 bp, synthetic construct
  misc_feature 1..639: note = Synthetic Construct
  source 1..639: mol_type = other DNA
gacatccagatgacccagagccccagcagcctgagcgccagcgtgggcgacagagtgaccatcacctgtcgggccagccagggcatccggaactacctggcctggtatcagcagaagcccggcaaggcccccaagctgctgatctacgccgccagcacactgcagagcggcgtgcccagcagattcagcggcagcggctccggcaccgacttcaccctgaccatcagcagcctgcagcccgaggacgtggccacctactactgccagcggtacaacagagccccctacaccttcggccagggcaccaaggtggaaatcaagggacagcccaaggctgccccctcggtcaccctgttccccccaagcagcgaggaactgcaggccaacaaggccaccctcgtgtgcctgatcagcgacttctaccctggcgccgtgaccgtggcctggaaggccgatagctctcccgtgaaggccggcgtggaaaccaccacccccagcaagcagagcaacaacaaatacgccgcctccagctacctgagcctgacccccgagcagtggaagtcccaccggtcctacagctgccaggtcacacacgagggcagcaccgtggaaaagaccgtggcccccaccgagtgcagc

SEQ ID NO: 66 - DNA, 1710 bp, synthetic construct
  misc_feature 1..1710: note = Synthetic Construct
  source 1..1710: mol_type = other DNA
caggtgcagctgcagcagagcggccctgagctggtcaagcctggcgccagcgtgaagatcagctgcaaggccagcggctacagcttcaccagctactggatccactggatcaagcagcggcctggccagggcctggaatggatcggcatgatcgaccccagcgacggcgagacacggctgaaccagagattccagggcagagccaccctgaccgtggacgagagcaccagcaccgcctacatgcagctgcggagccccaccagcgaggacagcgccgtgtactactgcacccggctgaaagagtacggcaactacgacagcttctacttcgacgtgtggggagccggcaccctggtcaccgtgtccagcgaagtgcagctgaaagaaagcggccctggcctggtggcccctggcggcagcctgagcatcacctgtaccgtgtccggcttcagcctgaccgacagcagcatcaactgggtccgacagccccctggcaagggcctcgagtggctgggaatgatctggggcgacggccggatcgactacgccgacgccctgaagtcccggctgagcatcagcaaggacagcagcaagagccaggtgttcctggaaatgaccagcctgcggaccgacgacaccgccacctactactgcgccagggacggctacttcccctacgccatggatttctggggccagggcaccagcgtgaccgtgtcctctgcttccaccaagggccccagcgtgttccctctggcccctagcagcaagagcacatctggcggaacagccgccctgggctgcctggtcaaggactactttcccgagcccgtgaccgtgtcctggaactctggtgccctgacaagcggagtgcataccttccctgccgtgctgcagagcagcggcctgtactctctgagcagcgtggtcaccgtgccaagcagcagcctgggcacccagacctacatctgcaacgtgaaccacaagccctccaacaccaaggtggacaagaaggtggaacccaagagctgcgacaagacccacacctgtcctccctgtcctgcccctgaactgctgggcggaccctccgtgttcctgttccctccaaagcccaaggataccctgatgatcagccggacccctgaagtgacctgcgtggtggtggacgtgtcccacgaggatcccgaagtgaagttcaattggtacgtggacggcgtggaagtgcataacgccaagaccaagcccagagaggaacagtacaacagcacctaccgggtggtgtccgtgctgacagtgctgcaccaggactggctgaacggcaaagagtacaagtgcaaggtgtccaacaaggccctgccagcccctatcgagaaaaccatcagcaaggccaagggccagccccgcgagcctcaggtgtacacactgcctccatgccgggacgagctgaccaagaaccaggtgtccctgtggtgcctcgtgaagggcttctacccctccgatatcgccgtggaatgggagagcaacggccagcccgagaacaactacaagaccacccctcccgtgctggacagcgacggctcattcttcctgtacagcaagctgaccgtggacaagtcccggtggcagcagggcaacgtgttcagctgctctgtgatgcacgaggccctgcacaaccggttcacccagaagtccctgagcctgagccctggc

SEQ ID NO: 67 - DNA, 1035 bp, synthetic construct
  misc_feature 1..1035: note = Synthetic Construct
  source 1..1035: mol_type = other DNA
gacatcgtgctgacccagagccctgccagcctggccgtgtctctgggccagagagccaccatcagctgccgggccagcgagagcgtggacagctacggccagagctacatgcactggtatcagcagaaggccggccagccccccaagctgctgatctacctggccagcaacctggaaagcggcgtgcccgccagattcagcggcagcggcagcagaaccgacttcaccctgaccatcgaccccgtgcaggccgaggacgccgccacctactactgccagcagaacgccgaggacagccggaccttcggcggaggcaccaagctggaaatcaagggcggctccggcagcagcggctctggcggcgatatccagatgacccagtcccccgcctccctgagcgtgtccgtgggcgacaccatcaccctgacatgccacgccagccagaacatcgacgtgtggctgagctggttccagcagaagcctggcaacatccctaagctgctcatctataaggcctccaacctgcacaccggcgtgcccagcaggttttccggctctggcagcggcaccggctttaccctgacaatcagcagcctgcagcccgaggatatcgccacatattactgtcagcaggcccacagctaccccttcacctttggcggcggaacaaagctcgagattaagggcggcagcggaagctccggctccggcggacgtacggtggccgctccttccgtgttcatcttccctccctccgacgagcagctgaagtccggcaccgcctccgtggtgtgtctgctgaacaacttctaccctcgggaggccaaggtgcagtggaaggtggacaacgccctgcagtccggcaactcccaggagtccgtcaccgagcaggactccaaggacagcacctactccctgtcctccaccctgaccctgtccaaggccgactacgagaagcacaaggtgtacgcctgtgaggtgacccaccagggcctgtccagccctgtgaccaagtccttcaaccggggcgagtgc

SEQ ID NO: 68 - protein, 570 aa, synthetic construct
  REGION 1..570: note = Synthetic Construct
EVQLKESGPGLVAPGGSLSITCTVSGFSLTDSSINWVRQPPGKGLEWLGMIWGDGRIDYADALKSRLSISKDSSKSQVFLEMTSLRTDDTATYYCARDGYFPYAMDFWGQGTSVTVSSQVQLQQSGPELVKPGASVKISCKASGYSFTSYWIHWIKQRPGQGLEWIGMIDPSDGETRLNQRFQGRATLTVDESTSTAYMQLRSPTSEDSAVYYCTRLKEYGNYDSFYFDVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 69 - protein, 345 aa, synthetic construct
  REGION 1..345: note = Synthetic Construct
DIQMTQSPASLSVSVGDTITLTCHASQNIDVWLSWFQQKPGNIPKLLIYKASNLHTGVPSRFSGSGSGTGFTLTISSLQPEDIATYYCQQAHSYPFTFGGGTKLEIKGGSGSSGSGGDIVLTQSPASLAVSLGQRATISCRASESVDSYGQSYMHWYQQKAGQPPKLLIYLASNLESGVPARFSGSGSRTDFTLTIDPVQAEDAATYYCQQNAEDSRTFGGGTKLEIKGGSGSSGSGGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - DNA, 1035 bp, synthetic construct
  misc_feature 1..1035: note = Synthetic Construct
  source 1..1035: mol_type = other DNA
gacatccagatgacccagagccccgccagcctgagcgtgtccgtgggcgataccatcaccctgacctgccacgccagccagaacatcgacgtgtggctgagctggttccagcagaagcccggcaacatccccaagctgctgatctacaaggccagcaacctgcacaccggcgtgcccagcagattcagcggctctggcagcggcaccggctttaccctgaccatcagcagcctgcagcccgaggatatcgccacctactactgccagcaggcccacagctaccccttcaccttcggcggaggcaccaagctggaaatcaagggcggcagcggcagctccggctctggcggcgatatcgtgctgacccagtctcccgcctccctggccgtgtctctgggccagagagccaccatcagctgccgggccagcgagagcgtggacagctacggccagagctacatgcactggtatcagcagaaggccggacagccccctaaactgctcatctacctggcctccaacctggaaagcggcgtgcccgccaggttttccggcagcggctccagaaccgacttcaccctgacaatcgaccccgtgcaggccgaggacgccgccacatattactgtcagcagaacgccgaggacagcagaacctttggcggcggaacaaagctcgagattaagggcggctccggctccagcggatctggcggacgtacggtggccgctccttccgtgttcatcttccctccctccgacgagcagctgaagtccggcaccgcctccgtggtgtgtctgctgaacaacttctaccctcgggaggccaaggtgcagtggaaggtggacaacgccctgcagtccggcaactcccaggagtccgtcaccgagcaggactccaaggacagcacctactccctgtcctccaccctgaccctgtccaaggccgactacgagaagcacaaggtgtacgcctgtgaggtgacccaccagggcctgtccagccctgtgaccaagtccttcaaccggggcgagtgc

SEQ ID NO: 71 - protein, 214 aa, synthetic construct
  REGION 1..214: note = Synthetic Construct
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - DNA, 642 bp, synthetic construct
  misc_feature 1..642: note = Synthetic Construct
  source 1..642: mol_type = other DNA
gacatccagatgacccagagccccagcagcctgagcgccagcgtgggcgacagagtgaccatcacctgtcgggccagccagggcatccggaactacctggcctggtatcagcagaagcccggcaaggcccccaagctgctgatctacgccgccagcacactgcagagcggcgtgcccagcagattcagcggcagcggctccggcaccgacttcaccctgaccatcagcagcctgcagcccgaggacgtggccacctactactgccagcggtacaacagagccccctacaccttcggccagggcaccaaggtggaaatcaagcgtacggtggccgctccttccgtgttcatcttccctccctccgacgagcagctgaagtccggcaccgcctccgtggtgtgtctgctgaacaacttctaccctcgggaggccaaggtgcagtggaaggtggacaacgccctgcagtccggcaactcccaggagtccgtcaccgagcaggactccaaggacagcacctactccctgtcctccaccctgaccctgtccaaggccgactacgagaagcacaaggtgtacgcctgtgaggtgacccaccagggcctgtccagccctgtgaccaagtccttcaaccggggcgagtgc

SEQ ID NO: 73 - protein, 447 aa, synthetic construct
  REGION 1..447: note = Synthetic Construct
EVQLKESGPGLVAPGGSLSITCTVSGFSLTDSSINWVRQPPGKGLEWLGMIWGDGRIDYADALKSRLSISKDSSKSQVFLEMTSLRTDDTATYYCARDGYFPYAMDFWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 74 - protein, 217 aa, synthetic construct
  REGION 1..217: note = Synthetic Construct
DIVLTQSPASLAVSLGQRATISCRASESVDSYGQSYMHWYQQKAGQPPKLLIYLASNLESGVPARFSGSGSRTDFTLTIDPVQAEDAATYYCQQNAEDSRTFGGGTKLEIKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 75 - protein, 573 aa, synthetic construct
  REGION 1..573: note = Synthetic Construct
QVQLQQSGPELVKPGASVKISCKASGYSFTSYWIHWIKQRPGQGLEWIGMIDPSDGETRLNQRFQGRATLTVDESTSTAYMQLRSPTSEDSAVYYCTRLKEYGNYDSFYFDVWGAGTLVTVSSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 76 - protein, 341 aa, synthetic construct
  REGION 1..341: note = Synthetic Construct
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKGGSGSSGSGGDIQMTQSPASLSVSVGDTITLTCHASQNIDVWLSWFQQKPGNIPKLLIYKASNLHTGVPSRFSGSGSGTGFTLTISSLQPEDIATYYCQQAHSYPFTFGGGTKLEIKGGSGSSGSGGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - DNA, 1341 bp, synthetic construct
  misc_feature 1..1341: note = Synthetic Construct
  source 1..1341: mol_type = other DNA
gaggtgcagctgaaggagagcggccccggcctggtggcccccggcggcagcctgagcatcacctgcaccgtgagcggcttcagcctgaccgacagcagcatcaactgggtgcgccagccccccggcaagggcctggagtggctgggcatgatctggggcgacggccgcatcgactacgccgacgccctgaagagccgcctgagcatcagcaaggacagcagcaagagccaggtgttcctggagatgaccagcctgcgcaccgacgacaccgccacctactactgcgcccgcgacggctacttcccctacgccatggacttctggggccagggcaccagcgtgaccgtgagcagcgccagcaccaagggccccagcgtgttccccctggcccctagcagcaagagcacatctggcggcacagccgccctgggctgcctggtcaaggactacttccccgagcccgtgacagtgtcctggaactctggcgccctgaccagcggagtgcataccttccctgccgtgctgcagtccagcggcctgtacagcctgagcagcgtggtcacagtgcccagcagcagcctgggcacccagacctacatctgcaacgtgaaccacaagcccagcaacaccaaggtggacaagaaggtggaacccaagagctgcgacaagacccacacctgtcccccctgccctgcccctgaactgctgggcggaccctccgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcataacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaagggccagcctagagagccccaagtctgcaccctgccccccagcagagatgagctgaccaagaaccaggtgtccctgagctgcgccgtgaagggcttctaccccagcgatatcgccgtggaatgggagagcaacggccagcccgagaacaactacaagaccaccccccctgtgctggacagcgacggctcattcttcctggtgtccaagctgacagtggacaagagccggtggcagcagggcaacgtgttcagctgcagcgtgatgcacgaggccctgcacaaccattacacccagaagtccctgagcctgagccccggc

SEQ ID NO: 78 - DNA, 651 bp, synthetic construct
  misc_feature 1..651: note = Synthetic Construct
  source 1..651: mol_type = other DNA
gacatcgtgctgacccagagccctgccagcctggccgtgtctctgggccagagagccaccatcagctgccgggccagcgagagcgtggacagctacggccagagctacatgcactggtatcagcagaaggccggccagccccccaagctgctgatctacctggccagcaacctggaaagcggcgtgcccgccagattcagcggcagcggcagcagaaccgacttcaccctgaccatcgaccccgtgcaggccgaggacgccgccacctactactgccagcagaacgccgaggacagccggaccttcggcggaggcaccaagctggaaatcaagggacagcccaaggctgccccctcggtcaccctgttccccccaagcagcgaggaactgcaggccaacaaggccaccctcgtgtgcctgatcagcgacttctaccctggcgccgtgaccgtggcctggaaggccgatagctctcccgtgaaggccggcgtggaaaccaccacccccagcaagcagagcaacaacaaatacgccgccagcagctacctgagcctgacccccgagcagtggaagtcccaccggtcctacagctgccaggtcacacacgagggcagcaccgtggaaaagaccgtggcccccaccgagtgcagc

SEQ ID NO: 79 - DNA, 1719 bp, synthetic construct
  misc_feature 1..1719: note = Synthetic Construct
  source 1..1719: mol_type = other DNA
caggtgcagctgcagcagagcggccctgagctggtcaagcctggcgccagcgtgaagatcagctgcaaggccagcggctacagcttcaccagctactggatccactggatcaagcagcggcctggccagggcctggaatggatcggcatgatcgaccccagcgacggcgagacacggctgaaccagagattccagggcagagccaccctgaccgtggacgagagcaccagcaccgcctacatgcagctgcggagccccaccagcgaggacagcgccgtgtactactgcacccggctgaaagagtacggcaactacgacagcttctacttcgacgtgtggggagccggcaccctggtcaccgtgtccagcgaagtgcagctggtggaaagcggcggaggcctggtgcagcccggcagaagcctgagactgagctgcgccgccagcggcttcaccttcgacgactacgccatgcactgggtccgacaggcccctggcaaaggactggaatgggtgtccgccatcacctggaacagcggccacatcgactacgccgacagcgtggaaggccggttcaccatcagccgggacaacgccaagaacagcctgtacctgcagatgaacagcctgcgggccgaggataccgccgtgtattattgcgccaaggtgtcctacctgagcaccgccagcagcctggactactggggccagggcaccctcgtgacagtgtcctccgcttccaccaagggccccagcgtgttccctctggcccctagcagcaagagcacatctggcggaacagccgccctgggctgcctggtcaaggactactttcccgagcccgtgaccgtgtcctggaactctggtgccctgacaagcggagtgcataccttccctgccgtgctgcagagcagcggcctgtactctctgagcagcgtggtcaccgtgccaagcagcagcctgggcacccagacctacatctgcaacgtgaaccacaagccctccaacaccaaggtggacaagaaggtggaacccaagagctgcgacaagacccacacctgtcctccctgtcctgcccctgaactgctgggcggaccctccgtgttcctgttccctccaaagcccaaggataccctgatgatcagccggacccctgaagtgacctgcgtggtggtggacgtgtcccacgaggatcccgaagtgaagttcaattggtacgtggacggcgtggaagtgcataacgccaagaccaagcccagagaggaacagtacaacagcacctaccgggtggtgtccgtgctgacagtgctgcaccaggactggctgaacggcaaagagtacaagtgcaaggtgtccaacaaggccctgccagcccctatcgagaaaaccatcagcaaggccaagggccagccccgcgagcctcaggtgtacacactgcctccatgccgggacgagctgaccaagaaccaggtgtccctgtggtgcctcgtgaagggcttctacccctccgatatcgccgtggaatgggagagcaacggccagcccgagaacaactacaagaccacccctcccgtgctggacagcgacggctcattcttcctgtacagcaagctgaccgtggacaagtcccggtggcagcagggcaacgtgttcagctgctctgtgatgcacgaggccctgcacaaccggttcacccagaagtccctgagcctgagccctggc

SEQ ID NO: 80 - DNA, 1023 bp, synthetic construct
  misc_feature 1..1023: note = Synthetic Construct
  source 1..1023: mol_type = other DNA
gacatccagatgacccagagccccagcagcctgagcgccagcgtgggcgacagagtgaccatcacctgtcgggccagccagggcatccggaactacctggcctggtatcagcagaagcccggcaaggcccccaagctgctgatctacgccgccagcacactgcagagcggcgtgcccagcagattcagcggcagcggctccggcaccgacttcaccctgaccatcagcagcctgcagcccgaggacgtggccacctactactgccagcggtacaacagagccccctacaccttcggccagggcaccaaggtggaaatcaagggcggctctggcagctccggcagcggcggagacattcagatgacacagtcccccgccagcctgtccgtgtccgtgggcgataccatcaccctgacatgccacgccagccagaacatcgacgtgtggctgagctggttccagcagaaacctggcaacatccctaagctgctcatctataaggccagcaacctgcacacaggcgtgccctccagattctccggctctggctctggcaccggctttacactgacaatcagttctctgcagcctgaggatatcgccacatattactgtcagcaggcccacagctaccctttcaccttcggaggcggcaccaagctcgagattaagggcggaagcggctcctccggctccggcggacgtacggtggccgctccttccgtgttcatcttccctccctccgacgagcagctgaagtccggcaccgcctccgtggtgtgtctgctgaacaacttctaccctcgggaggccaaggtgcagtggaaggtggacaacgccctgcagtccggcaactcccaggagtccgtcaccgagcaggactccaaggacagcacctactccctgtcctccaccctgaccctgtccaaggccgactacgagaagcacaaggtgtacgcctgtgaggtgacccaccagggcctgtccagccctgtgaccaagtccttcaaccggggcgagtgc

SEQ ID NO: 81 - protein, 573 aa, synthetic construct
  REGION 1..573: note = Synthetic Construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSQVQLQQSGPELVKPGASVKISCKASGYSFTSYWIHWIKQRPGQGLEWIGMIDPSDGETRLNQRFQGRATLTVDESTSTAYMQLRSPTSEDSAVYYCTRLKEYGNYDSFYFDVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 82 - protein, 341 aa, synthetic construct
  REGION 1..341: note = Synthetic Construct
DIQMTQSPASLSVSVGDTITLTCHASQNIDVWLSWFQQKPGNIPKLLIYKASNLHTGVPSRFSGSGSGTGFTLTISSLQPEDIATYYCQQAHSYPFTFGGGTKLEIKGGSGSSGSGGDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKGGSGSSGSGGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - DNA, 1719 bp, synthetic construct
  misc_feature 1..1719: note = Synthetic Construct
  source 1..1719: mol_type = other DNA
gaggtgcagctggtggaaagcggcggaggactggtgcagcccggcagaagcctgagactgagctgcgccgccagcggcttcaccttcgacgactacgccatgcactgggtccgacaggcccctggcaagggcctggaatgggtgtccgccatcacctggaacagcggccacatcgactacgccgacagcgtggaaggccggttcaccatcagccgggacaacgccaagaacagcctgtacctgcagatgaacagcctgcgggccgaggacaccgccgtgtactactgcgccaaggtgtcctacctgagcaccgccagcagcctggactactggggccagggcaccctggtcaccgtgtccagtcaggtccagctgcagcagagcggccctgagctggtcaagcctggcgccagcgtgaagatcagctgcaaggccagcggctacagcttcaccagctactggatccactggatcaagcagcggcctggccagggcctcgagtggatcggcatgatcgaccccagcgacggcgagacacggctgaaccagagattccagggcagagccaccctgaccgtggacgagagcaccagcaccgcctacatgcagctgcggagccccaccagcgaggatagcgccgtgtattattgcacccggctgaaagagtacggcaactacgacagcttctacttcgacgtgtggggagccggcaccctcgtgacagtgtcctccgcttccaccaagggccccagcgtgttccctctggcccctagcagcaagagcacatctggcggaacagccgccctgggctgcctggtcaaggactactttcccgagcccgtgaccgtgtcctggaactctggtgccctgacaagcggagtgcataccttccctgccgtgctgcagagcagcggcctgtactctctgagcagcgtggtcaccgtgccaagcagcagcctgggcacccagacctacatctgcaacgtgaaccacaagccctccaacaccaaggtggacaagaaggtggaacccaagagctgcgacaagacccacacctgtcctccctgtcctgcccctgaactgctgggcggaccctccgtgttcctgttccctccaaagcccaaggataccctgatgatcagccggacccctgaagtgacctgcgtggtggtggacgtgtcccacgaggatcccgaagtgaagttcaattggtacgtggacggcgtggaagtgcataacgccaagaccaagcccagagaggaacagtacaacagcacctaccgggtggtgtccgtgctgacagtgctgcaccaggactggctgaacggcaaagagtacaagtgcaaggtgtccaacaaggccctgccagcccctatcgagaaaaccatcagcaaggccaagggccagccccgcgagcctcaggtgtacacactgcctccatgccgggacgagctgaccaagaaccaggtgtccctgtggtgcctcgtgaagggcttctacccctccgatatcgccgtggaatgggagagcaacggccagcccgagaacaactacaagaccacccctcccgtgctggacagcgacggctcattcttcctgtacagcaagctgaccgtggacaagtcccggtggcagcagggcaacgtgttcagctgctctgtgatgcacgaggccctgcacaaccggttcacccagaagtccctgagcctgagccctggc

SEQ ID NO: 84 - DNA, 1023 bp, synthetic construct
  misc_feature 1..1023: note = Synthetic Construct
  source 1..1023: mol_type = other DNA
gacatccagatgacccagagccccgccagcctgagcgtgtccgtgggcgataccatcaccctgacctgccacgccagccagaacatcgacgtgtggctgagctggttccagcagaagcccggcaacatccccaagctgctgatctacaaggccagcaacctgcacaccggcgtgcccagcagattcagcggctctggcagcggcaccggctttaccctgaccatcagcagcctgcagcccgaggatatcgccacctactactgccagcaggcccacagctaccccttcaccttcggcggaggcaccaagctggaaatcaagggcggcagcggcagctccggcagcggcggagacattcagatgacacagtcccccagcagcctgtccgccagcgtgggcgacagagtgaccatcacctgtcgggccagccagggcatccggaactacctggcctggtatcagcagaaacctggcaaggcccctaaactgctcatctacgccgccagcacactgcagtctggcgtgccctccagattctccggaagcggctccggcaccgatttcaccctgacaatctcatctctgcagcctgaggacgtggccacatattactgccagagatacaacagagccccctacacctttggccagggcaccaaggtcgagattaagggcggatccggctccagcggcagcggaggacgtacggtggccgctccttccgtgttcatcttccctccctccgacgagcagctgaagtccggcaccgcctccgtggtgtgtctgctgaacaacttctaccctcgggaggccaaggtgcagtggaaggtggacaacgccctgcagtccggcaactcccaggagtccgtcaccgagcaggactccaaggacagcacctactccctgtcctccaccctgaccctgtccaaggccgactacgagaagcacaaggtgtacgcctgtgaggtgacccaccagggcctgtccagccctgtgaccaagtccttcaaccggggcgagtgc

SEQ ID NO: 85 - protein, 452 aa, synthetic construct
  REGION 1..452: note = Synthetic Construct
QVQLQQSGPELVKPGASVKISCKASGYSFTSYWIHWIKQRPGQGLEWIGMIDPSDGETRLNQRFQGRATLTVDESTSTAYMQLRSPTSEDSAVYYCTRLKEYGNYDSFYFDVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 86 - protein, 213 aa, synthetic construct
  REGION 1..213: note = Synthetic Construct
DIQMTQSPASLSVSVGDTITLTCHASQNIDVWLSWFQQKPGNIPKLLIYKASNLHTGVPSRFSGSGSGTGFTLTISSLQPEDIATYYCQQAHSYPFTFGGGTKLEIKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 87 - protein, 568 aa, synthetic construct
  REGION 1..568: note = Synthetic Construct
EVQLKESGPGLVAPGGSLSITCTVSGFSLTDSSINWVRQPPGKGLEWLGMIWGDGRIDYADALKSRLSISKDSSKSQVFLEMTSLRTDDTATYYCARDGYFPYAMDFWGQGTSVTVSSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 88 - protein, 344 aa, synthetic construct
  REGION 1..344: note = Synthetic Construct
IQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKGGSGSSGSGGDIVLTQSPASLAVSLGQRATISCRASESVDSYGQSYMHWYQQKAGQPPKLLIYLASNLESGVPARFSGSGSRTDFTLTIDPVQAEDAATYYCQQNAEDSRTFGGGTKLEIKGGSGSSGSGGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - DNA, 1356 bp, synthetic construct
  misc_feature 1..1356: note = Synthetic Construct
  source 1..1356: mol_type = other DNA
caggtgcagctgcagcagagcggccccgagctggtgaagcccggcgccagcgtgaagatcagctgcaaggccagcggctacagcttcaccagctactggattcactggatcaagcagcgccccggccagggcctggagtggatcggcatgatcgaccccagcgacggcgagacccgcctgaaccagcgcttccagggccgcgccaccctgaccgtggacgagagcaccagcaccgcctacatgcagctgcgcagccccaccagcgaggacagcgccgtgtactactgcacccgcctgaaggagtacggcaactacgacagcttctacttcgacgtgtggggcgccggcaccctggtgaccgtgagcagcgccagcaccaagggccccagcgtgttccccctggcccctagcagcaagagcacatctggcggcacagccgccctgggctgcctggtcaaggactacttccccgagcccgtgacagtgtcctggaactctggcgccctgaccagcggagtgcataccttccctgccgtgctgcagtccagcggcctgtacagcctgagcagcgtggtcacagtgcccagcagcagcctgggcacccagacctacatctgcaacgtgaaccacaagcccagcaacaccaaggtggacaagaaggtggaacccaagagctgcgacaagacccacacctgtcccccctgccctgcccctgaactgctgggcggaccctccgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcataacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaagggccagcctagagagccccaagtctgcaccctgccccccagcagagatgagctgaccaagaaccaggtgtccctgagctgcgccgtgaagggcttctaccccagcgatatcgccgtggaatgggagagcaacggccagcccgagaacaactacaagaccaccccccctgtgctggacagcgacggctcattcttcctggtgtccaagctgacagtggacaagagccggtggcagcagggcaacgtgttcagctgcagcgtgatgcacgaggccctgcacaaccattacacccagaagtccctgagcctgagccccggc

SEQ ID NO: 90 - DNA, 639 bp, synthetic construct
  misc_feature 1..639: note = Synthetic Construct
  source 1..639: mol_type = other DNA
gacatccagatgacccagagccccgccagcctgagcgtgtccgtgggcgataccatcaccctgacctgccacgccagccagaacatcgacgtgtggctgagctggttccagcagaagcccggcaacatccccaagctgctgatctacaaggccagcaacctgcacaccggcgtgcccagcagattcagcggctctggcagcggcaccggctttaccctgaccatcagcagcctgcagcccgaggatatcgccacctactactgccagcaggcccacagctaccccttcaccttcggcggaggcaccaagctggaaatcaagggacagcccaaggctgccccctcggtcaccctgttccccccaagctctgaggaactgcaggccaacaaggccaccctcgtgtgcctgatcagcgacttctaccctggcgccgtgaccgtggcctggaaggccgatagctctcccgtgaaggccggcgtggaaaccaccacccccagcaagcagagcaacaacaaatacgccgccagcagctacctgagcctgacccccgagcagtggaagtcccaccggtcctacagctgccaggtcacacacgagggcagcaccgtggaaaagaccgtggcccccaccgagtgcagc

SEQ ID NO: 91 - DNA, 1704 bp, synthetic construct
  misc_feature 1..1704: note = Synthetic Construct
  source 1..1704: mol_type = other DNA
gaggtgcagctgaaagagtccggccctggactggtggcccctggcggcagcctgagcatcacctgtaccgtgtccggcttcagcctgaccgacagcagcatcaactgggtccgacagccccctggcaagggcctggaatggctgggcatgatctggggcgacggccggatcgactacgccgacgccctgaagtcccggctgagcatcagcaaggacagcagcaagagccaggtgttcctggaaatgaccagcctgcggaccgacgacaccgccacctactactgcgccagggacggctacttcccctacgccatggatttctggggccagggcaccagcgtgaccgtgtcctccgaagtgcagctggtggaaagcggcggaggcctggtgcagcccggcagaagcctgagactgagctgcgccgccagcggcttcaccttcgacgactacgccatgcactgggtccgccaggctcccggaaagggactcgagtgggtgtccgccatcacctggaacagcggccacatcgattacgccgatagcgtggaaggccggttcaccatcagccgggacaacgccaagaacagcctgtacctgcagatgaacagcctgagagccgaggataccgccgtgtactactgtgccaaggtgtcctacctgagcaccgccagcagcctggactactggggacagggaaccctggtcaccgtgtccagcgcttccaccaagggccccagcgtgttccctctggcccctagcagcaagagcacatctggcggaacagccgccctgggctgcctggtcaaggactactttcccgagcccgtgaccgtgtcctggaactctggtgccctgacaagcggagtgcataccttccctgccgtgctgcagagcagcggcctgtactctctgagcagcgtggtcaccgtgccaagcagcagcctgggcacccagacctacatctgcaacgtgaaccacaagccctccaacaccaaggtggacaagaaggtggaacccaagagctgcgacaagacccacacctgtcctccctgtcctgcccctgaactgctgggcggaccctccgtgttcctgttccctccaaagcccaaggataccctgatgatcagccggacccctgaagtgacctgcgtggtggtggacgtgtcccacgaggatcccgaagtgaagttcaattggtacgtggacggcgtggaagtgcataacgccaagaccaagcccagagaggaacagtacaacagcacctaccgggtggtgtccgtgctgacagtgctgcaccaggactggctgaacggcaaagagtacaagtgcaaggtgtccaacaaggccctgccagcccctatcgagaaaaccatcagcaaggccaagggccagccccgcgagcctcaggtgtacacactgcctccatgccgggacgagctgaccaagaaccaggtgtccctgtggtgcctcgtgaagggcttctacccctccgatatcgccgtggaatgggagagcaacggccagcccgagaacaactacaagaccacccctcccgtgctggacagcgacggctcattcttcctgtacagcaagctgaccgtggacaagtcccggtggcagcagggcaacgtgttcagctgctctgtgatgcacgaggccctgcacaaccggttcacccagaagtccctgagcctgagccctggc

SEQ ID NO: 92 - DNA, 1035 bp, synthetic construct
  misc_feature 1..1035: note = Synthetic Construct
  source 1..1035: mol_type = other DNA
gacatccagatgacccagagccccagcagcctgagcgccagcgtgggcgacagagtgaccatcacctgtcgggccagccagggcatccggaactacctggcctggtatcagcagaagcccggcaaggcccccaagctgctgatctacgccgccagcacactgcagagcggcgtgcccagcagattcagcggcagcggctccggcaccgacttcaccctgaccatcagcagcctgcagcccgaggacgtggccacctactactgccagcggtacaacagagccccctacaccttcggccagggcaccaaggtggaaatcaagggcggctctggcagctccggctctggcggcgatatcgtgctgacccagtctcccgccagcctggccgtgtctctgggccagagagccaccatcagctgcagagccagcgagagcgtggacagctacggccagagctacatgcattggtatcagcagaaagccggccagcctcctaaactgctcatctacctggccagcaacctggaatccggcgtgcccgccaggttttccggcagcggcagcagaaccgatttcacactgacaatcgaccccgtgcaggccgaggatgccgccacatattactgtcagcagaacgccgaggacagccggaccttcggcggaggcaccaagctcgagattaagggcggaagcggctccagcggcagtggcggacgtacggtggccgctccttccgtgttcatcttccctccctccgacgagcagctgaagtccggcaccgcctccgtggtgtgtctgctgaacaacttctaccctcgggaggccaaggtgcagtggaaggtggacaacgccctgcagtccggcaactcccaggagtccgtcaccgagcaggactccaaggacagcacctactccctgtcctccaccctgaccctgtccaaggccgactacgagaagcacaaggtgtacgcctgtgaggtgacccaccagggcctgtccagccctgtgaccaagtccttcaaccggggcgagtgc

SEQ ID NO: 93 - protein, 568 aa, synthetic construct
  REGION 1..568: note = Synthetic Construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSEVQLKESGPGLVAPGGSLSITCTVSGFSLTDSSINWVRQPPGKGLEWLGMIWGDGRIDYADALKSRLSISKDSSKSQVFLEMTSLRTDDTATYYCARDGYFPYAMDFWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 94 - protein, 345 aa, synthetic construct
  REGION 1..345: note = Synthetic Construct
DIVLTQSPASLAVSLGQRATISCRASESVDSYGQSYMHWYQQKAGQPPKLLIYLASNLESGVPARFSGSGSRTDFTLTIDPVQAEDAATYYCQQNAEDSRTFGGGTKLEIKGGSGSSGSGGDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKGGSGSSGSGGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - DNA, 1704 bp, synthetic construct
  misc_feature 1..1704: note = Synthetic Construct
  source 1..1704: mol_type = other DNA
gaggtgcagctggtggaaagcggcggaggactggtgcagcccggcagaagcctgagactgagctgcgccgccagcggcttcaccttcgacgactacgccatgcactgggtccgacaggcccctggcaagggcctggaatgggtgtccgccatcacctggaacagcggccacatcgactacgccgacagcgtggaaggccggttcaccatcagccgggacaacgccaagaacagcctgtacctgcagatgaacagcctgcgggccgaggacaccgccgtgtactactgcgccaaggtgtcctacctgagcaccgccagcagcctggactactggggccagggcaccctggtcaccgtgtcctccgaagtgcagctgaaagagtccggccctggcctggtggcccctggcggcagcctgagcatcacctgtaccgtgtccggcttcagcctgaccgacagcagcatcaactgggtccgccagcctcccggaaagggactcgagtggctgggcatgatctggggcgacggccggatcgattacgccgatgccctgaagtcccggctgagcatcagcaaggacagcagcaagagccaggtgttcctggaaatgaccagcctgagaaccgacgacaccgccacctactactgtgcccgggacggctacttcccctacgccatggatttctggggacagggaaccagcgtgaccgtgtccagcgcttccaccaagggccccagcgtgttccctctggcccctagcagcaagagcacatctggcggaacagccgccctgggctgcctggtcaaggactactttcccgagcccgtgaccgtgtcctggaactctggtgccctgacaagcggagtgcataccttccctgccgtgctgcagagcagcggcctgtactctctgagcagcgtggtcaccgtgccaagcagcagcctgggcacccagacctacatctgcaacgtgaaccacaagccctccaacaccaaggtggacaagaaggtggaacccaagagctgcgacaagacccacacctgtcctccctgtcctgcccctgaactgctgggcggaccctccgtgttcctgttccctccaaagcccaaggataccctgatgatcagccggacccctgaagtgacctgcgtggtggtggacgtgtcccacgaggatcccgaagtgaagttcaattggtacgtggacggcgtggaagtgcataacgccaagaccaagcccagagaggaacagtacaacagcacctaccgggtggtgtccgtgctgacagtgctgcaccaggactggctgaacggcaaagagtacaagtgcaaggtgtccaacaaggccctgccagcccctatcgagaaaaccatcagcaaggccaagggccagccccgcgagcctcaggtgtacacactgcctccatgccgggacgagctgaccaagaaccaggtgtccctgtggtgcctcgtgaagggcttctacccctccgatatcgccgtggaatgggagagcaacggccagcccgagaacaactacaagaccacccctcccgtgctggacagcgacggctcattcttcctgtacagcaagctgaccgtggacaagtcccggtggcagcagggcaacgtgttcagctgctctgtgatgcacgaggccctgcacaaccggttcacccagaagtccctgagcctgagccctggc

SEQ ID NO: 96 - DNA, 1035 bp, synthetic construct
  misc_feature 1..1035: note = Synthetic Construct
  source 1..1035: mol_type = other DNA
gacatcgtgctgacccagagccctgccagcctggccgtgtctctgggccagagagccaccatcagctgccgggccagcgagagcgtggacagctacggccagagctacatgcactggtatcagcagaaggccggccagccccccaagctgctgatctacctggccagcaacctggaaagcggcgtgcccgccagattcagcggcagcggcagcagaaccgacttcaccctgaccatcgaccccgtgcaggccgaggacgccgccacctactactgccagcagaacgccgaggacagccggaccttcggcggaggcaccaagctggaaatcaagggcggctccggcagcagcggctctggcggcgatatccagatgacccagtcccccagcagcctgagcgccagcgtgggcgacagagtgaccatcacctgtagagccagccagggcatccggaactacctggcttggtatcagcagaaacccggaaaggcccctaaactgctcatctacgccgccagcaccctgcagtccggcgtgccaagcagattctccggctctggcagcggcaccgatttcacactgacaatcagcagcctgcagcccgaggatgtggccacctattattgccagagatacaacagagccccctacaccttcggccagggcaccaaggtcgagattaagggcggaagcggcagctccggctccggcggacgtacggtggccgctccttccgtgttcatcttccctccctccgacgagcagctgaagtccggcaccgcctccgtggtgtgtctgctgaacaacttctaccctcgggaggccaaggtgcagtggaaggtggacaacgccctgcagtccggcaactcccaggagtccgtcaccgagcaggactccaaggacagcacctactccctgtcctccaccctgaccctgtccaaggccgactacgagaagcacaaggtgtacgcctgtgaggtgacccaccagggcctgtccagccctgtgaccaagtccttcaaccggggcgagtgc

SEQ ID NO: 97 - DNA, 1710 bp, synthetic construct
  misc_feature 1..1710: note = Synthetic Construct
  source 1..1710: mol_type = other DNA
gaggtgcagctgaaagagtccggccctggactggtggcccctggcggcagcctgagcatcacctgtaccgtgtccggcttcagcctgaccgacagcagcatcaactgggtccgacagccccctggcaagggcctggaatggctgggcatgatctggggcgacggccggatcgactacgccgacgccctgaagtcccggctgagcatcagcaaggacagcagcaagagccaggtgttcctggaaatgaccagcctgcggaccgacgacaccgccacctactactgcgccagggacggctacttcccctacgccatggatttctggggccagggcaccagcgtgaccgtgtccagtcaggtccagctgcagcagagcggccctgagctggtcaagcctggcgccagcgtgaagatcagctgcaaggccagcggctacagcttcaccagctactggatccactggatcaagcagcggcctggccagggcctcgagtggatcggaatgatcgaccccagcgacggcgagacacggctgaaccagagattccagggcagagccaccctgaccgtggacgagagcaccagcaccgcctacatgcagctgcggagccccaccagcgaggacagcgccgtgtactactgcacccggctgaaagaatacggcaactacgacagcttctacttcgacgtgtggggagccggcaccctggtcaccgtgtctagcgcttccaccaagggccccagcgtgttccctctggcccctagcagcaagagcacatctggcggaacagccgccctgggctgcctggtcaaggactactttcccgagcccgtgaccgtgtcctggaactctggtgccctgacaagcggagtgcataccttccctgccgtgctgcagagcagcggcctgtactctctgagcagcgtggtcaccgtgccaagcagcagcctgggcacccagacctacatctgcaacgtgaaccacaagccctccaacaccaaggtggacaagaaggtggaacccaagagctgcgacaagacccacacctgtcctccctgtcctgcccctgaactgctgggcggaccctccgtgttcctgttccctccaaagcccaaggataccctgatgatcagccggacccctgaagtgacctgcgtggtggtggacgtgtcccacgaggatcccgaagtgaagttcaattggtacgtggacggcgtggaagtgcataacgccaagaccaagcccagagaggaacagtacaacagcacctaccgggtggtgtccgtgctgacagtgctgcaccaggactggctgaacggcaaagagtacaagtgcaaggtgtccaacaaggccctgccagcccctatcgagaaaaccatcagcaaggccaagggccagccccgcgagcctcaggtgtacacactgcctccatgccgggacgagctgaccaagaaccaggtgtccctgtggtgcctcgtgaagggcttctacccctccgatatcgccgtggaatgggagagcaacggccagcccgagaacaactacaagaccacccctcccgtgctggacagcgacggctcattcttcctgtacagcaagctgaccgtggacaagtcccggtggcagcagggcaacgtgttcagctgctctgtgatgcacgaggccctgcacaaccggttcacccagaagtccctgagcctgagccctggc

SEQ ID NO: 98 - protein, 4 aa, synthetic construct
  REGION 1..4: note = Synthetic Construct
GGGG

SEQ ID NO: 99 - protein, 5 aa, synthetic construct
  REGION 1..5: note = Synthetic Construct
GGGGG

SEQ ID NO: 100 - protein, 6 aa, synthetic construct
  REGION 1..6: note = Synthetic Construct
GGGGGG

SEQ ID NO: 101 - protein, 7 aa, synthetic construct
  REGION 1..7: note = Synthetic Construct
GGGGGGG

SEQ ID NO: 102 - protein, 8 aa, synthetic construct
  REGION 1..8: note = Synthetic Construct
GGGGGGGG

SEQ ID NO: 103 - protein, 5 aa, synthetic construct
  REGION 1..5: note = Synthetic Construct
GGGGS

SEQ ID NO: 104 - protein, 10 aa, synthetic construct
  REGION 1..10: note = Synthetic Construct
GGGGSGGGGS

SEQ ID NO: 105 - protein, 15 aa, synthetic construct
  REGION 1..15: note = Synthetic Construct
GGGGSGGGGSGGGGS

SEQ ID NO: 106 - protein, 5 aa, synthetic construct
  REGION 1..5: note = Synthetic Construct
TKGPS

SEQ ID NO: 107 - protein, 5 aa, synthetic construct
  REGION 1..5: note = Synthetic Construct
TVAAP

SEQ ID NO: 108 - protein, 5 aa, synthetic construct
  REGION 1..5: note = Synthetic Construct
QPKAA

SEQ ID NO: 109 - protein, 5 aa, synthetic construct
  REGION 1..5: note = Synthetic Construct
QRIEG

SEQ ID NO: 110 - protein, 7 aa, synthetic construct
  REGION 1..7: note = Synthetic Construct
ASTKGPS

SEQ ID NO: 111 - protein, 7 aa, synthetic construct
  REGION 1..7: note = Synthetic Construct
RTVAAPS

SEQ ID NO: 112 - protein, 7 aa, synthetic construct
  REGION 1..7: note = Synthetic Construct
GQPKAAP

SEQ ID NO: 113 - protein, 7 aa, synthetic construct
  REGION 1..7: note = Synthetic Construct
HIDSPNK

SEQ ID NO: 114 - protein, 448 aa, synthetic construct
  REGION 1..448: note = Synthetic Construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLGK

SEQ ID NO: 115 - protein, 213 aa, synthetic construct
  REGION 1..213: note = Synthetic Construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - DNA, 1344 bp, synthetic construct
  misc_feature 1..1344: note = Synthetic Construct
  source 1..1344: mol_type = other DNA
caggtgcagctgcagcagcctggcgccgaactcgtgaaacctggcgcctccgtgaagatgagctgcaaggccagcggctacaccttcaccagctacaacatgcactgggtcaagcagacccccggcagaggcctggaatggatcggcgccatctaccccggcaacggcgacacctcctacaaccagaagttcaagggcaaggccaccctgaccgccgacaagagcagcagcacagcctacatgcagctgtccagcctgaccagcgaggacagcgccgtgtactactgcgccagaagcacctactacggcggcgactggtacttcaacgtgtggggagccggcaccaccgtgacagtgtctgctgcttcgaccaagggcccatcggtgttccctctggccccttgcagcagaagcaccagcgaatctacagccgccctgggctgcctcgtgaaggactactttcccgagcccgtgaccgtgtcctggaactctggcgctctgacaagcggcgtgcacacctttccagccgtgctccagagcagcggcctgtactctctgagcagcgtcgtgacagtgcccagcagcagcctgggcaccaagacctacacctgtaacgtggaccacaagcccagcaacaccaaggtggacaagcgggtggaatctaagtacggccctccctgccctccttgcccagcccctgaatttctgggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgaccgtgctgcaccaggactggctgaacggcaaagagtacaagtgcaaggtgtccaacaagggcctgcccagctccatcgagaaaaccatcagcaaggccaagggccagccccgcgagcctcaagtgtataccctgcccccttgccaggaagagatgaccaagaaccaggtgtccctgtggtgtctcgtgaaaggcttctaccccagcgacattgccgtggaatgggagagcaacggccagcccgagaacaactacaagaccaccccccctgtgctggacagcgacggctcattcttcctgtactccaagctgaccgtggacaagagccggtggcaggaaggcaacgtgttcagctgctccgtgatgcacgaggccctgcacaaccactacacccagaagtccctgtctctgtccctgggcaag

SEQ ID NO: 117 - DNA, 639 bp, synthetic construct
  misc_feature 1..639: note = Synthetic Construct
  source 1..639: mol_type = other DNA
cagatcgtgctgagccagagccctgccatcctgagcgcttccccaggcgagaaagtgaccatgacctgcagagccagcagcagcgtgtcctacatccactggttccagcagaagcccggcagcagccccaagccttggatctacgccaccagcaatctggccagcggagtgcctgtgcggtttagcggctctggcagcggcacaagctacagcctgaccatcagccgggtggaagccgaagatgccgccacctactactgccagcagtggaccagcaacccccccacatttggcggaggcaccaagctggaaatcaagcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgt

SEQ ID NO: 118 - DNA, 1713 bp, synthetic construct; identical to SEQ ID NO: 11
  misc_feature 1..1713: note = Synthetic Construct
  source 1..1713: mol_type = other DNA

SEQ ID NO: 119 - DNA, 1026 bp, synthetic construct; identical to SEQ ID NO: 12
  misc_feature 1..1026: note = Synthetic Construct
  source 1..1026: mol_type = other DNA

SEQ ID NO: 120 - protein, 8 aa, synthetic construct
  REGION 1..8: note = Synthetic Construct
GYTFTSYN

SEQ ID NO: 121 - protein, 8 aa, synthetic construct
  REGION 1..8: note = Synthetic Construct
IYPGNGDT

SEQ ID NO: 122 - protein, 14 aa, synthetic construct
  REGION 1..14: note = Synthetic Construct
ARSTYYGGDWYFNV

SEQ ID NO: 123 - protein, 5 aa, synthetic construct
  REGION 1..5: note = Synthetic Construct
SSVSY

SEQ ID NO: 124 - skipped

SEQ ID NO: 125 - protein, 7 aa, synthetic construct
  REGION 1..7: note = Synthetic Construct
QQWTSNP

SEQ ID NO: 126 - protein, 10 aa, synthetic construct
  REGION 1..10: note = Synthetic Construct
GYIFTNYNIH

SEQ ID NO: 127 - protein, 10 aa, synthetic construct
  REGION 1..10: note = Synthetic Construct
AIYPGNGDAP

SEQ ID NO: 128 - protein, 9 aa, synthetic construct
  REGION 1..9: note = Synthetic Construct
ANWDVAFAY

SEQ ID NO: 129 - protein, 10 aa, synthetic construct
  REGION 1..10: note = Synthetic Construct
GFTFDDYAMH

SEQ ID NO: 130 - protein, 10 aa, synthetic construct
  REGION 1..10: note = Synthetic Construct
AITWNSGHID

SEQ ID NO: 131 - protein, 12 aa, synthetic construct
  REGION 1..12: note = Synthetic Construct
VSYLSTASSLDY

SEQ ID NO: 132 - protein, 10 aa, synthetic construct
  REGION 1..10: note = Synthetic Construct
GYSFTSYWIH

SEQ ID NO: 133 - protein, 9 aa, synthetic construct
  REGION 1..9: note = Synthetic Construct
MIDPSDGET

SEQ ID NO: 134 - protein, 14 aa, synthetic construct
  REGION 1..14: note = Synthetic Construct
LKEYGNYDSFYFDV

SEQ ID NO: 135 - protein, 10 aa, synthetic construct
  REGION 1..10: note = Synthetic Construct
GFSLTDSSIN

SEQ ID NO: 136 - protein, 9 aa, synthetic construct
  REGION 1..9: note = Synthetic Construct
MIWGDGRID

SEQ ID NO: 137 - protein, 10 aa, synthetic construct
  REGION 1..10: note = Synthetic Construct
DGYFPYAMDF

SEQ ID NO: 138 - protein, 11 aa, synthetic construct
  REGION 1..11: note = Synthetic Construct
KASQDIDRYMA

SEQ ID NO: 139 - protein, 7 aa, synthetic construct
  REGION 1..7: note = Synthetic Construct
DTSTLQS

SEQ ID NO: 140 - protein, 8 aa, synthetic construct
  REGION 1..8: note = Synthetic Construct
LQYDNLWT

SEQ ID NO: 141 - protein, 11 aa, synthetic construct
  REGION 1..11: note = Synthetic Construct
RASQGIRNYLA

SEQ ID NO: 142 - protein, 9 aa, synthetic construct
  REGION 1..9: note = Synthetic Construct
QRYNRAPYT

SEQ ID NO: 143 - protein, 11 aa, synthetic construct
  REGION 1..11: note = Synthetic Construct
HASQNIDVWLS

SEQ ID NO: 144 - protein, 9 aa, synthetic construct
  REGION 1..9: note = Synthetic Construct
QQAHSYPFT

SEQ ID NO: 145 - protein, 15 aa, synthetic construct
  REGION 1..15: note = Synthetic Construct
RASESVDSYGQSYMH

SEQ ID NO: 146 - protein, 7 aa, synthetic construct
  REGION 1..7: note = Synthetic Construct
LASNLES

SEQ ID NO: 147 - protein, 9 aa, synthetic construct
  REGION 1..9: note = Synthetic Construct
QQNAEDSRT

SEQ ID NO: 148 - protein, 10 aa, synthetic construct
  REGION 1..10: note = Synthetic Construct
GGSGSSGSGG

SEQ ID NO: 149 - protein, 11 aa, synthetic construct
  REGION 1..11: note = Synthetic Construct
QSLVHNNGNTY

SEQ ID NO: 150 - protein, 120 aa, synthetic construct [sequence omitted]

SEQ ID NO: 151 - protein, 107 aa, synthetic construct [sequence omitted]

SEQ ID NO: 152 - protein, 121 aa, synthetic construct
  REGION 1..121: note = Synthetic Construct
QVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSS

SEQ ID NO: 153 - protein, 112 aa, synthetic construct
  REGION 1..112: note = Synthetic Construct
DIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIK

SEQ ID NO: 154 - protein, 121 aa, synthetic construct
  REGION 1..121: note = Synthetic Construct
QVQLVESGGGVVQPGRSLRLSCAASGFTFTKAWMHWVRQAPGKGLEWVAQIKDKSNSYATYYADSVKGRFTISRDNSKNTLYLQMNSLRAEDTAVYYCRGVYYALSPFDYWGQGTLVTVSS

SEQ ID NO: 155 - protein, 112 aa, synthetic construct
  REGION 1..112: note = Synthetic Construct
DIVMTQTPLSLSVTPGQPASISCKSSQSLVHNNGNTYLSWYLQKPGQSPQLLIYKVSNRFSGVPDRFSGSGSGTDFTLKISRVEAEDVGVYYCGQGTQYPFTFGGGTKVEIK

SEQ ID NO: 156 - protein, 124 aa, synthetic construct
  REGION 1..124: note = Synthetic Construct
QVQLVQSGAEVKKPGSSVKVSCKASGYAFSSYWMNWVRQAPGQGLEWIGQIWPGDGDTNYNQKFKGRATLTADESTSTAYMELSSLRSEDTAVYYCARRETTTVGRYYYAMDYWGQGTTVTVSS

SEQ ID NO: 157 - protein, 111 aa, synthetic construct
  REGION 1..111: note = Synthetic Construct
DLVLTQSPASLAVSPGQRATITCKASQSVDYDGDSYLNWYQQKPGQPPKLLIYDASNLVSGVPARFSGSGSGTDFTLTINPVEANDTANYYCQQSTEDPWTFGQGTKLEIK

SEQ ID NO: 158 - protein, 121 aa, synthetic construct [sequence omitted]

SEQ ID NO: 159 - protein, 106 aa, synthetic construct [sequence omitted]

SEQ ID NO: 160 - protein, 120 aa, synthetic construct
  REGION 1..120: note = Synthetic Construct
QVQLVQSGAEVVKPGASVKVSCKASGYTFTSYYIHWVRQAPGQGLEWIGSIYPGNVNTNYAQKFQGRATLTVDTSISTAYMELSRLRSDDTAVYYCTRSHYGLDWNFDVWGKGTTVTVSS

SEQ ID NO: 161 - protein, 107 aa, synthetic construct
  REGION 1..107: note = Synthetic Construct
DIQMTQSPSSLSASVGDRVTITCQASQNIYVWLNWYQQKPGKAPKLLIYKASNLHTGVPSRFSGSGSGTDFTLTISSLQPEDIATYYCQQGQTYPYTFGQGTKLEIK

SEQ ID NO: 162 - protein, 120 aa, synthetic construct
  REGION 1..120: note = Synthetic Construct
QVQLQESGPGLVKPSQTLSLTCTVSGFSLSDYGVHWVRQPPGKGLEWLGVIWAGGGTNYNPSLKSRKTISKDTSKNQVSLKLSSVTAADTAVYYCARDKGYSYYYSMDYWGQGTTVTVSS

SEQ ID NO: 163 - protein, 111 aa, synthetic construct
  REGION 1..111: note = Synthetic Construct
DIVLTQSPASLAVSPGQRATITCRASESVEYYVTSLMQWYQQKPGQPPKLLIFAASNVESGVPARFSGSGSGTDFTLTINPVEANDVANYYCQQSRKVPYTFGQGTKLEIK

SEQ ID NO: 164 - protein, 120 aa, synthetic construct [sequence omitted]

SEQ ID NO: 165 - protein, 107 aa, synthetic construct [sequence omitted]

SEQ ID NO: 166 - protein, 118 aa, synthetic construct
  REGION 1..118: note = Synthetic Construct
QVQLVQSGAEVKKPGSSVKVSCKASGYIFTNYNIHWVKKSPGQGLEWIGAIYPGNGDAPYSQKFQGKATLTADTSTSTTYMELSSLRSEDTAVYYCVRANWDVAFAYWGQGTLVTVSS

SEQ ID NO: 167 - protein, 106 aa, synthetic construct
  REGION 1..106: note = Synthetic Construct
DIQMTQSPSSLSASVGDRVTITCKASQDIDRYMAWYQDKPGKAPRLLIHDTSTLQSGVPSRFSGSGSGRDYTLTISNLEPEDFATYYCLQYDNLWTFGGGTKVEIK

SEQ ID NO: 168 - protein, 121 aa, synthetic construct [sequence omitted]

SEQ ID NO: 169 - protein, 107 aa, synthetic construct [sequence omitted]

SEQ ID NO: 170 - protein, 123 aa, synthetic construct
  REGION 1..123: note = Synthetic Construct
QVQLQQSGPELVKPGASVKISCKASGYSFTSYWIHWIKQRPGQGLEWIGMIDPSDGETRLNQRFQGRATLTVDESTSTAYMQLRSPTSEDSAVYYCTRLKEYGNYDSFYFDVWGAGTLVTVSS

SEQ ID NO: 171 - protein, 107 aa, synthetic construct
  REGION 1..107: note = Synthetic Construct
DIQMTQSPASLSVSVGDTITLTCHASQNIDVWLSWFQQKPGNIPKLLIYKASNLHTGVPSRFSGSGSGTGFTLTISSLQPEDIATYYCQQAHSYPFTFGGGTKLEIK

SEQ ID NO: 172 - protein, 118 aa, synthetic construct
  REGION 1..118: note = Synthetic Construct
EVQLKESGPGLVAPGGSLSITCTVSGFSLTDSSINWVRQPPGKGLEWLGMIWGDGRIDYADALKSRLSISKDSSKSQVFLEMTSLRTDDTATYYCARDGYFPYAMDFWGQGTSVTVSS

SEQ ID NO: 173 - protein, 111 aa, synthetic construct
  REGION 1..111: note = Synthetic Construct
DIVLTQSPASLAVSLGQRATISCRASESVDSYGQSYMHWYQQKAGQPPKLLIYLASNLESGVPARFSGSGSRTDFTLTIDPVQAEDAATYYCQQNAEDSRTFGGGTKLEIK

SEQ ID NO: 174 - protein, 5 aa, synthetic construct
  REGION 1..5: note = Synthetic Construct
  VARIANT 1..5: note = Can be absent or present, or present in repeats of up to 5 times
GGGGS

SEQ ID NO: 175 - protein, 7 aa, synthetic construct
  REGION 1..7: note = Synthetic Construct
GQPKAAP

SEQ ID NO: 176 - protein, 5 aa, synthetic construct
  REGION 1..5: note = Synthetic Construct
DKTHT

SEQ ID NO: 177 - protein, 6 aa, synthetic construct
  REGION 1..6: note = Synthetic Construct
TKGPSR

SEQ ID NO: 178 - protein, 7 aa, synthetic construct
  REGION 1..7: note = Synthetic Construct
AASTLQS

SEQ ID NO: 179 - protein, 7 aa, synthetic construct
  REGION 1..7: note = Synthetic Construct
KASNLHT